(19) United States
(12) Patent Application Publication
(10) Pub. No.: US 2024/0101546 A1
(43) Pub. Date: Mar. 28, 2024

(54) HETEROARYL-ACETYLENES, PHARMACEUTICAL COMPOSITIONS THEREOF, AND THEIR THERAPEUTIC APPLICATIONS

(71) Applicant: Eubulus Biotherapeutics Inc., Jiaxing (CN)

(72) Inventors: Sheldon Cao, San Diego, CA (US); Chaoran Huang, Auburndale, MA (US); Xiaolie Wang, Shanghai (CN)

(21) Appl. No.: 18/255,855

(22) PCT Filed: Dec. 3, 2022
(86) PCT No.: PCT/CN2021/135247

§ 371 Date: Jun. 2, 2023

Related U.S. Application Data
(60) Provisional application No. 63/121,300, filed on Dec. 4, 2020.

(51) Int. Cl. C07D 417/12; C07D 513/04; C07D 277/44; C07D 417/14; C07D 401/14; C07D 471/04; A61P 35/00

(52) U.S. Cl. CPC C07D 417/12; C07D 513/04; C07D 277/44; C07D 417/14; C07D 401/14; C07D 471/04; A61P 35/00

(57) ABSTRACT

Provided herein are heteroaryl-acetylene compounds, e.g., a compound of Formula (I), and pharmaceutical compositions thereof. Also provided herein are methods of their use for treating, preventing, or ameliorating one or more symptoms of GPX4-mediated disorders, diseases, or conditions.

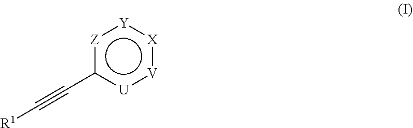

### CROSS REFERENCE TO RELATED APPLICATION

This application claims the benefit of the priority of U.S. Provisional Application No. 63/121,300, filed Dec. 4, 2020; the disclosure of which is incorporated herein by reference in its entirety.

### FIELD

Provided herein are heteroaryl-acetylene compounds and pharmaceutical compositions thereof. Also provided herein are methods of their use for treating, preventing, or ameliorating one or more symptoms of a GPX4-mediated disorder, disease, or condition.

### BACKGROUND

Regulated cell death is essential for the survival of a multicellular organism. Dixon et al., Cell 2012, 149, 1060-72; Fearnhead et al., Cell Death Differ. 2017, 24, 1991-98; Gudipaty et al., Annu. Rev. Cell Dev. Biol. 2018, 34, 311-32; Mou et al., J. Hematol. Oncol. 2019, 12, 34. Ferroptosis is one type of regulated cell death characterized by loss of glutathione peroxidase 4 (GPX4) activity and accumulation of lipid peroxides. Dixon et al., Cell 2012, 149, 1060-72; Yang et al., Cell 2014, 156, 317-31. Ferroptosis dysfunction have been observed in many types of cancer, including breast cancer, colorectal cancer, diffuse large B-cell lymphoma, gastric cancer, hepatocellular carcinoma, lung cancer, and ovarian cancer. Mou et al., J. Hematol. Oncol. 2019, 12, 34.

GPX4, a selenoenzyme, is a negative regulator of ferroptosis. Yang et al., Cell 2014, 156, 317-31; Seibt et al., Free Radic. Biol. Med. 2019, 133, 144-52. GPX4 catalyzes the reduction of lipid peroxides and prevents ferroptosis. Brigelius-Flohe and Maiorino, Biochim. Biophys. Acta 2013, 1830, 3289-303; Cao and Dixon, Cell. Mol. Life Sci. 2016, 73, 2195-209. Small molecule GPX4 inhibitors (e.g., RSL3 and ML162) have been shown to be able to induce ferroptosis and suppress tumor growth in xenograft models. Yang et al., Cell 2014, 156, 317-31; Lei et al., Front. Physiol. 2019, 10, 139; Bi et al., Cell Death Disease 2019, 10, 682.

Despite the advances in cancer treatment, cancer remains a major worldwide public health problem. It was estimated that there will be 1,806,590 new cancer cases diagnosed and 606,520 cancer deaths in the US alone in 2020. Cancer Facts & Figures 2020. Therefore, there is a need for an effective therapy for cancer treatment.

### SUMMARY OF THE DISCLOSURE

Provided herein is a compound of Formula (I):

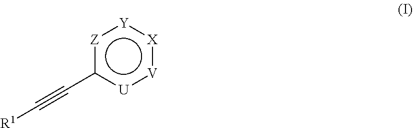

or an enantiomer, a mixture of enantiomers, a diastereomer, a mixture of two or more diastereomers, a tautomer, a mixture of two or more tautomers, or an isotopic variant thereof; or
a pharmaceutically acceptable salt, solvate, hydrate, or prodrug thereof; wherein;
U, V, X, and Z are each independently —C(R^{2a})═, —N═, —N(R^{2b})—, —O—, —S—, or -A-L¹-L²-R³; with the proviso that at least one of U and Z is —N═, and one of U, V, X, and Z is -A-L¹-L²-R³;
Y is a bond, —C(R^{2a})═, or —N═;
A is —C(O)—, —C(O)NR^{1a}—, —OC(O)NR^{1a}—, —NR^{1a}C(O)NR^{1d}—, —S(O)—, —S(O)₂—, —S(O)NR^{1a}—, or —S(O)₂NR^{1a}—;
L¹ is a bond, C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
L² is C₆₋₁₄ arylene, heteroarylene, or heterocyclylene;
R¹ is hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₈ aralkyl, heteroaryl, or heterocyclyl;
R³ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2a} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2b} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₈ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{h} and R^{c} together with the N atom to which they are attached form heterocyclyl optionally substituted with one or more, in one embodiment, one, two, three, or four, substituents Q^{a};
wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

Also provided herein is a pharmaceutical composition comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

Additionally provided herein is a method of treating, preventing, or ameliorating one or more symptoms of a proliferative disease in a subject, comprising administering to the subject a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Furthermore, provided herein is a method of treating, preventing, or ameliorating one or more symptoms of a disorder, disease, or condition mediated by a glutathione peroxidase 4 (GPX4) in a subject, comprising administering to the subject a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting the growth of a cell, comprising contacting the cell with a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inducing ferroptosis in a cell, comprising contacting the cell with a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of irreversibly inhibiting the activity of a protein, comprising contacting the protein with a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

Provided herein is a method of inhibiting the activity of a GPX4, comprising contacting the GPX4 with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, biochemistry, biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and a biological molecule (e.g., a protein, enzyme, RNA, or DNA), cell, or tissue such that a physiological and/or chemical effect takes place as a result of such contact. Contacting can take place in vitro, ex vivo, or in vivo. In one embodiment, a therapeutic agent is contacted with a biological molecule in vitro to determine the effect of the therapeutic agent on the biological molecule. In another embodiment, a therapeutic agent is contacted with a cell in cell culture (in vitro) to determine the effect of the therapeutic agent on the cell. In yet another embodiment, the contacting of a therapeutic agent with a biological molecule, cell, or tissue includes the administration of a therapeutic agent to a subject having the biological molecule, cell, or tissue to be contacted.

The term “therapeutically effective amount” or “effective amount” is meant to include the amount of a compound that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound that is required for 50% inhibition of a maximal response in an assay that measures such a response.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, and commensurate with a reasonable benefit/risk ratio. See, e.g., Remington: The Science and Practice of Pharmacy, 22nd ed.; Allen Ed.; Pharmaceutical Press: London, 2012; Handbook of Pharmaceutical Excipients, 8th ed.; Sheskey et al., Eds.: Pharmaceutical Press: London, 2017; Handbook of Pharmaceutical Additives, 3rd ed.; Ash and Ash Eds.: Synapse Information Resources: 2007; Pharmaceutical Preformulation and Formulation, 2nd ed.; Gibson Ed.: Drugs and the Pharmaceutical Sciences 199; Informa Healthcare: New York, NY, 2009.

The term “about” or “approximately” means an acceptable error for a particular value as determined by one of ordinary skill in the art, which depends in part on how the value is measured or determined. In certain embodiments, the term “about” or “approximately” means within 1, 2, or 3 standard deviations. In certain embodiments, the term “about” or “approximately” means within 25%, 20%, 15%, 10%, 9%, 8%, 7%, 6%, 5%, 4%, 3%, 2%, 1%, 0.5%, or 0.05% of a given value or range.

The term “alkyl” refers to a linear or branched saturated monovalent hydrocarbon radical, wherein the alkyl is optionally substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms, e.g., n-propyl and isopropyl), butyl (including all isomeric forms, e.g., n-butyl, isobutyl, sec-butyl, and t-butyl), pentyl (including all isomeric forms, e.g., n-pentyl, isopentyl, sec-pentyl, neopentyl, and tert-pentyl), and hexyl (including all isomeric forms, e.g., n-hexyl, isohexyl, and sec-hexyl).

The terms “alkylene” and “alkanediyl” are used interchangeably herein in reference to a linear or branched saturated divalent hydrocarbon radical, wherein the alkanediyl is optionally be substituted with one or more substituents Q as described herein. For example, C₁₋₆ alkanediyl refers to a linear saturated divalent hydrocarbon radical of 1 to 6 carbon atoms or a branched saturated divalent hydrocarbon radical of 3 to 6 carbon atoms. In certain embodiments, the alkanediyl is a linear saturated divalent hydrocarbon radical that has 1 to 30 (C₁₋₃₀), 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₁₀ alkanediyl groups are also referred as “lower alkanediyl.” Examples of alkanediyl groups include, but are not limited to, methanediyl, ethanediyl (including all isomeric forms, e.g., ethane-1,1-diyl and ethane-1,2-diyl), propanediyl (including all isomeric forms, e.g., propane-1,1-diyl, propane-1,2-diyl, and propane-1,3-diyl), butanediyl (including all isomeric forms, e.g., butane-1,1-diyl, butane-1,2-diyl, butane-1,3-diyl, and butane-1,4-diyl), pentanediyl (including all isomeric forms, e.g., pentane-1,1-diyl, pentane-1,2-diyl, pentane-1,3-diyl, and pentane-1,5-diyl), and hexanediyl (including all isomeric forms, e.g., hexane-1,1-diyl, hexane-1,2-diyl, hexane-1,3-diyl, and hexane-1,6-diyl). Examples of substituted alkanediyl groups include, but are not limited to, —C(O)CH₂—, —C(O)(CH₂)₂—, —C(O)(CH₂)₃—, —C(O)(CH₂)₄—, —C(O)(CH₂)₅—, —C(O)(CH₂)₆—, —C(O)(CH₂)₇—, —C(O)(CH₂)₈—, —C(O)(CH₂)₉—, —C(O)(CH₂)₁₀—, —C(O)CH₂C(O)—, —C(O)(CH₂)₂C(O)—, —C(O)(CH₂)₃C(O)—, —C(O)(CH₂)₄C(O)—, or —C(O)(CH₂)₅C(O)—.

The term “alkenyl” refers to a linear or branched monovalent hydrocarbon radical, which contains one or more, in one embodiment, one, two, three, or four, in another embodiment, one, carbon-carbon double bond(s). The alkenyl is optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propenyl (including all isomeric forms, e.g., propen-1-yl, propen-2-yl, and allyl), and butenyl (including all isomeric forms, e.g., buten-1-yl, buten-2-yl, buten-3-yl, and 2-buten-1-yl).

The terms “alkenylene” and “alkenediyl” are used interchangeably herein in reference to a linear or branched divalent hydrocarbon radical, which contains one or more, in one embodiment, one, two, three, or four, in another embodiment, one, carbon-carbon double bond(s). The alkenediyl is optionally substituted with one or more substituents Q as described herein. The term “alkenediyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenediyl refers to a linear unsaturated divalent hydrocarbon radical of 2 to 6 carbon atoms or a branched unsaturated divalent hydrocarbon radical of 3 to 6 carbon atoms. In certain embodiments, the alkenediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 30 (C₃₋₃₀), 3 to 20 (C₃₋₂₀), 3 to 15 (C₃₋₁₅), 3 to 10 (C₃₋₁₀), or 3 to 6 (C₃₋₆) carbon atoms. Examples of alkenediyl groups include, but are not limited to, ethenediyl (including all isomeric forms, e.g., ethene-1,1-diyl and ethene-1,2-diyl), propenediyl (including all isomeric forms, e.g., 1-propene-1,1-diyl, 1-propene-1,2-diyl, and 1-propene-1,3-diyl), butenediyl (including all isomeric forms, e.g., 1-butene-1,1-diyl, 1-butene-1,2-diyl, and 1-butene-1,4-diyl), pentenediyl (including all isomeric forms, e.g., 1-pentene-1,1-diyl, 1-pentene-1,2-diyl, and 1-pentene-1,5-diyl), and hexenediyl (including all isomeric forms, e.g., 1-hexene-1,1-diyl, 1-hexene-1,2-diyl, 1-hexene-1,3-diyl, 1-hexene-1,4-diyl, 1-hexene-1,5-diyl, and 1-hexene-1,6-diyl).

The term “alkynyl” refers to a linear or branched monovalent hydrocarbon radical, which contains one or more, in one embodiment, one, two, three, or four, in another embodiment, one, carbon-carbon triple bond(s). The alkynyl is optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 4 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 4 to 20 (C₄₋₂₀), 4 to 15 (C₄₋₁₅), 4 to 10 (C₄₋₁₀), or 4 to 6 (C₄₋₂₀) carbon atoms. Examples of alkynyl groups include, but are not limited to, ethynyl (—C═CH), propynyl (including all isomeric forms, e.g., 1-propynyl (—C≡CCH₃) and propargyl (—CH₂C═CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl and 2-hexyn-1-yl).

The terms “alkynylene” and “alkynediyl” are used interchangeably herein in reference to a linear or branched divalent hydrocarbon radical, which contains one or more, in one embodiment, one, two, three, or four, in another embodiment, one, carbon-carbon triple bond(s). The alkynediyl is optionally substituted with one or more substituents Q as described herein. For example, C₂₋₆ alkynediyl refers to a linear unsaturated divalent hydrocarbon radical of 2 to 6 carbon atoms or a branched unsaturated divalent hydrocarbon radical of 4 to 6 carbon atoms. In certain embodiments, the alkynediyl is a linear divalent hydrocarbon radical of 2 to 30 (C₂₋₃₀), 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 4 to 30 (C₄₋₃₀), 4 to 20 (C₄₋₂₀), 4 to 15 (C₄₋₁₅), 4 to 10 (C₄₋₁₀), or 4 to 6 (C₄₋₆) carbon atoms. Examples of alkynediyl groups include, but are not limited to, ethynediyl, propynediyl (including all isomeric forms, e.g., 1-propyne-1,3-diyl and 1-propyne-3,3-diyl), butynediyl (including all isomeric forms, e.g., 1-butyne-1,3-diyl, 1-butyne-1,4-diyl, and 2-butyne-1,1-diyl), pentynediyl (including all isomeric forms, e.g., 1-pentyne-1,3-diyl, 1-pentyne-1,4-diyl, and 2-pentyne-1,1-diyl), and hexynediyl (including all isomeric forms, e.g., 1-hexyne-1,3-diyl, 1-hexyne-1,4-diyl, and 2-hexyne-1,1-diyl).

The term “cycloalkyl” refers to a cyclic monovalent hydrocarbon radical, which is optionally substituted with one or more substituents Q as described herein. In one embodiment, the cycloalkyl is a saturated or unsaturated but non-aromatic, and/or bridged or non-bridged, and/or fused bicyclic group. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. In one embodiment, the cycloalkyl is monocyclic. In another embodiment, the cycloalkyl is bicyclic. In yet another embodiment, the cycloalkyl is tricyclic. In still another embodiment, the cycloalkyl is polycyclic. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[1.1.1]pentyl, bicyclo[2.1.1]hexyl, bicyclo[2.2.1]heptyl, bicyclo[2.2.2]octyl, decalinyl, and adamantyl.

The terms “cycloalkylene” and “cycloalkanediyl” are used interchangeably herein in reference to a cyclic divalent hydrocarbon radical, which may be optionally substituted with one or more substituents Q as described herein. In one embodiment, cycloalkanediyl groups may be saturated or unsaturated but non-aromatic, and/or bridged, and/or non-bridged, and/or fused bicyclic groups. In certain embodiments, the cycloalkanediyl has from 3 to 30 (C₃₋₃₀), 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkanediyl groups include, but are not limited to, cyclopropanediyl (including all isomeric forms, e.g., cyclopropane-1,1-diyl and cyclopropane-1,2-diyl), cyclobutanediyl (including all isomeric forms, e.g., cyclobutane-1,1-diyl, cyclobutane-1,2-diyl, and cyclobutane-1,3-diyl), cyclopentanediyl (including all isomeric forms, e.g., cyclopentane-1,1-diyl, cyclopentane-1,2-diyl, and cyclopentane-1,3-diyl), cyclohexanediyl (including all isomeric forms, e.g., cyclohexane-1,1-diyl, cyclohexane-1,2-diyl, cyclohexane-1,3-diyl, and cyclohex-1,4-diyl), cycloheptanediyl (including all isomeric forms, e.g., cycloheptane-1,1-diyl, cycloheptane-1,2-diyl, cycloheptane-1,3-diyl, and cycloheptane-1,4-diyl), decalinediyl (including all isomeric forms, e.g., decaline-1,1-diyl, decaline-1,2-diyl, and decaline-1,8-diyl), and adamantdiyl (including all isomeric forms, e.g., adamant-1,2-diyl, adamant-1,3-diyl, and adamant-1,8-diyl).

The term “aryl” refers to a monovalent monocyclic aromatic hydrocarbon radical and/or monovalent polycyclic aromatic hydrocarbon radical that contain at least one aromatic carbon ring. In certain embodiments, the aryl has from 6 to 20 (C₆₋₂₀), from 6 to 15 (C₆₋₁₅), or from 6 to 10 (C₆₋₁₀) ring carbon atoms. Examples of aryl groups include, but are not limited to, phenyl, naphthyl, fluorenyl, azulenyl, anthryl, phenanthryl, pyrenyl, biphenyl, and terphenyl. The aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In one embodiment, the aryl is monocyclic. In another embodiment, the aryl is bicyclic. In yet another embodiment, the aryl is tricyclic. In still another embodiment, the aryl is polycyclic. In certain embodiments, the aryl is optionally substituted with one or more substituents Q as described herein.

The terms “arylene” and “arenediyl” are used interchangeably herein in reference to a divalent monocyclic aromatic hydrocarbon radical or divalent polycyclic aromatic hydrocarbon radical that contains at least one aromatic hydrocarbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene (including all isomeric forms, e.g., phen-1,2-diyl, phen-1,3-diyl, and phen-1,4-diyl), naphthylene (including all isomeric forms, e.g., naphth-1,2-diyl, naphth-1,3-diyl, and naphth-1,8-diyl), fluorenylene (including all isomeric forms, e.g., fluoren-1,2-diyl, fluoren-1,3-diyl, and fluoren-1,8-diyl), azulenylene (including all isomeric forms, e.g., azulen-1,2-diyl, azulen-1,3-diyl, and azulen-1,8-diyl), anthrylene (including all isomeric forms, e.g., anthr-1,2-diyl, anthr-1,3-diyl, and anthr-1,8-diyl), phenanthrylene (including all isomeric forms, e.g., phenanthr-1,2-diyl, phenanthr-1,3-diyl, and phenanthr-1,8-diyl), pyrenylene (including all isomeric forms, e.g., pyren-1,2-diyl, pyren-1,3-diyl, and pyren-1,8-diyl), biphenylene (including all isomeric forms, e.g., biphen-2,3-diyl, biphen-3,4′-diyl, and biphen-4,4′-diyl), and terphenylene (including all isomeric forms. e.g., terphen-2,3-diyl, terphen-3,4′-diyl, and terphen-4,4′-diyl). Arylene also refers to bicyclic or tricyclic carbon rings, where one of the rings is aromatic and the others of which may be saturated, partially unsaturated, or aromatic, for example, dihydronaphthylene (including all isomeric forms. e.g., dihydronaphth-1,2-diyl and dihydronaphth-1,8-diyl), indenylene (including all isomeric forms, e.g., inden-1,2-diyl, inden-1,5-diyl, and inden-1,7-diyl), indanylene (including all isomeric forms, e.g., indan-1,2-diyl, indan-1,5-diyl, and indan-1,7-diyl), or tetrahydronaphthylene (tetralinylene) (including all isomeric forms, e.g., tetrahydronaphth-1,2-diyl, tetrahydronaphth-1,5-diyl, and tetrahydronaphth-1,8-diyl). In certain embodiments, arylene is optionally substituted with one or more substituents Q as described herein.

The terms “aralkyl” and “arylalkyl” are used interchangeably herein in reference to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, phenylethyl (including all isomeric forms, e.g., 1-phenylethyl and 2-phenylethyl), and phenylpropyl (including all isomeric forms, e.g., 1-phenylpropyl, 2-phenylpropyl, and 3-phenylpropyl). In certain embodiments, the aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each independently selected from O, S, and N, in the ring. The heteroaryl is bonded to the rest of a molecule through the aromatic ring. Each ring of a heteroaryl group can contain one or two O atoms, one or two S atoms, and/or one to four N atoms; provided that the total number of heteroatoms in each ring is four or less and each ring contains at least one carbon atom. In certain embodiments, the heteroaryl has from 5 to 20, from 5 to 15, or from 5 to 10 ring atoms. In one embodiment, the heteroaryl is monocyclic. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. In another embodiment, the heteroaryl is bicyclic. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyrindyl (including all isomeric forms, e.g., furo[2,3-b]pyridinyl, furo[2,3-c]pyridinyl, furo[3,2-b]pyridinyl, furo[3,2-c]pyridinyl, furo[3,4-b]pyridinyl, and furo[3,4-c]pyridinyl), imidazopyridinyl (including all isomeric forms. e.g., imidazo[1,2-a]pyridinyl, imidazo[4,5-b]pyridinyl, and imidazo[4,5-c]pyridinyl), imidazothiazolyl (including all isomeric forms, e.g., imidazo[2,1-b]thiazolyl and imidazo[4,5-d]thiazolyl), indazolyl, indolizinyl, indolyl, isobenzofuranyl, isobenzothienyl (i.e., benzo[c]thienyl), isoindolyl, isoquinolinyl, naphthyridinyl (including all isomeric forms, e.g., 1,5-naphthyridinyl, 1,6-naphthyridinyl, 1,7-naphthyridinyl, and 1,8-naphthyridinyl), oxazolopyridinyl (including all isomeric forms, e.g., oxazolo[4,5-b]pyridinyl, oxazolo[4,5-c]pyridinyl, oxazolo[5,4-b]pyridinyl, and oxazolo[5,4-c]pyridinyl), phthalazinyl, pteridinyl, purinyl, pyrrolopyridyl (including all isomeric forms, e.g., pyrrolo[2,3-b]pyridinyl, pyrrolo[2,3-c]pyridinyl, pyrrolo[3,2-b]pyridinyl, and pyrrolo[3,2-c]pyridinyl), quinolinyl, quinoxalinyl, quinazolinyl, thiadiazolopyrimidyl (including all isomeric forms, e.g., [1,2,5]thiadiazolo[3,4-d]pyrimidinyl and [1,2,3]thiadiazolo[4,5-d]pyrimidinyl), and thienopyridyl (including all isomeric forms, e.g., thieno[2,3-b]pyridinyl, thieno[2,3-c]pyridinyl, thieno[3,2-b]pyridinyl, and thieno[3,2-c]pyridinyl). In yet another embodiment, the heteroaryl is tricyclic. Examples of tricyclic heteroaryl groups include, but are not limited to, acridinyl, benzindolyl, carbazolyl, dibenzofuranyl, perimidinyl, phenanthrolinyl, phenanthridinyl (including all isomeric forms, e.g., 1,5-phenanthrolinyl, 1,6-phenanthrolinyl, 1,7-phenanthrolinyl, 1,9-phenanthrolinyl, and 2,10-phenanthrolinyl), phenarsazinyl, phenazinyl, phenothiazinyl, phenoxazinyl, and xanthenyl. In certain embodiments, the heteroaryl is optionally substituted with one or more substituents Q as described herein.

The terms “heteroarylene” and “heteroarenediyl” are used interchangeably herein in reference to a divalent monocyclic aromatic group or divalent polycyclic aromatic group that contains at least one aromatic ring, wherein at least one aromatic ring contains one or more heteroatoms in the ring, each of which is independently selected from O, S, and N. A heteroarylene group has at least one linkage to the rest of a molecule via its aromatic ring(s). Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furandiyl, imidazoldiyl, isothiazoldiyl, isoxazoldiyl, oxadiazoldiyl, oxazoldiyl, pyrazindiyl, pyrazoldiyl, pyridazindiyl, pyridindiyl, pyrimidindiyl, pyrroldiyl, thiadiazoldiyl, thiazoldiyl, thiendiyl, tetrazoldiyl, triazinediyl, and triazoldiyl. Examples of bicyclic heteroarylene groups include, but are not limited to, benzofurandiyl, benzimidazoldiyl, benzoisoxazoldiyl, benzopyrandiyl, benzothiadiazoldiyl, benzothiazoldiyl, benzothiendiyl, benzotriazoldiyl, benzoxazoldiyl, furopyridindiyl (including all isomeric forms. e.g., furo[2,3-b]pyridindiyl, furo[2,3-c]pyridindiyl, furo[3,2-b]pyridindiyl, furo[3,2-c]pyridindiyl, furo[3,4-b]pyridindiyl, and furo[3,4-c]pyridindiyl), imidazopyridindiyl (including all isomeric forms, e.g., imidazo[1,2-a]pyridindiyl, imidazo[4,5-b]pyridindiyl, and imidazo[4,5-c]pyridindiyl), imidazothiazoldiyl (including all isomeric forms, e.g., imidazo[2,1-b]thiazoldiyl and imidazo[4,5-d]thiazoldiyl), indazoldiyl, indolizindiyl, indoldiyl, isobenzofurandiyl, isobenzothiendiyl (i.e., benzo[c]thiendiyl), isoindoldiyl, isoquinolindiyl, naphthyridindiyl (including all isomeric forms, e.g., 1,5-naphthyridindiyl, 1,6-naphthyridindiyl, 1,7-naphthyridindiyl, and 1,8-naphthyridindiyl), oxazolopyridindiyl (including all isomeric forms, e.g., oxazolo[4,5-b]pyridindiyl, oxazolo[4,5-c]pyridindiyl, oxazolo[5,4-b]pyridindiyl, and oxazolo[5,4-c]pyridindiyl), phthalazindiyl, pteridindiyl, purindiyl, pyrrolopyridindiyl (including all isomeric forms, e.g., pyrrolo[2,3-b]pyridindiyl, pyrrolo[2,3-c]pyridindiyl, pyrrolo[3,2-b]pyridindiyl, and pyrrolo[3,2-c]pyridindiyl), quinolindiyl, quinoxalindiyl, quinazolindiyl, thiadiazolopyrimidindiyl (including all isomeric forms, e.g., [1,2,5]thiadiazolo[3,4-d]pyrimidindiyl and [1,2,3]thiadiazolo[4,5-d]pyrimidindiyl), and thienopyridindiyl (including all isomeric forms, e.g., thieno[2,3-b]pyridindiyl, thieno[2,3-c]pyridindiyl, thieno[3,2-b]pyridindiyl, and thieno[3,2-c]pyridindiyl). Examples of tricyclic heteroarylene groups include, but are not limited to, acridindiyl, benzindoldiyl, carbazoldiyl, dibenzofurandiyl, perimidindiyl, phenanthrolindiyl (including all isomeric forms, e.g., 1,5-phenanthrolindiyl, 1,6-phenanthrolindiyl, 1,7-phenanthrolindiyl, 1,9-phenanthrolindiyl, and 2,10-phenanthrolindiyl), phenanthridindiyl, phenarsazindiyl, phenazindiyl, phenothiazindiyl, phenoxazindiyl, and xanthendiyl. In certain embodiments, heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. The heterocyclyl is bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of heterocyclyls and heterocyclic groups include, but are not limited to, azepinyl, benzodioxanyl, benzodioxolyl, benzofuranonyl, chromanyl, decahydroisoquinolinyl, dihydrobenzofuranyl, dihydrobenzisothiazolyl, dihydrobenzisoxazinyl (including all isomeric forms, e.g., 1,4-dihydrobenzo[d][1,3]oxazinyl, 3,4-dihydrobenzo[c][1,2]-oxazinyl, and 3,4-dihydrobenzo[d][1,2]oxazinyl), dihydrobenzothienyl, dihydroisobenzofuranyl, dihydrobenzo[c]thienyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isochromanyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, thiochromanyl, tetrahydroquinolinyl, and 1,3,5-trithianyl. In certain embodiments, the heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. Heterocyclylene groups are bonded to the rest of a molecule through the non-aromatic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepindiyl, benzodioxandiyl, benzodioxoldiyl, benzofuranondiyl, chromandiyl, decahydroisoquinolindiyl, dihydrobenzofurandiyl, dihydrobenzisothiazoldiyl, dihydrobenzisoxazindiyl (including all isomeric forms, e.g., 1,4-dihydrobenzo[d][1,3]oxazindiyl, 3,4-dihydrobenzo[c][1,2]oxazindiyl, and 3,4-dihydrobenzo[d][1,2]oxazindiyl), dihydrobenzothiendiyl, dihydroisobenzofurandiyl, dihydrobenzo[c]thiendiyl, dihydrofurdiyl, dihydroisoindoldiyl, dihydropyrandiyl, dihydropyrazoldiyl, dihydropyrazindiyl, dihydropyridindiyl, dihydropyrimidindiyl, dihydropyrroldiyl, dioxolandiyl, 1,4-dithiandiyl, furanondiyl, imidazolidindiyl, imidazolindiyl, indolindiyl, isochromandiyl, isoindolindiyl, isothiazolidindiyl, isoxazolidindiyl, morpholindiyl, octahydroindoldiyl, octahydroisoindoldiyl, oxazolidinondiyl, oxazolidindiyl, oxirandiyl, piperazindiyl, piperidindiyl, 4-piperidondiyl, pyrazolidindiyl, pyrazolindiyl, pyrrolidindiyl, pyrrolindiyl, quinuclidindiyl, tetrahydrofurdiyl, tetrahydroisoquinolindiyl, tetrahydropyrandiyl, tetrahydrothiendiyl, thiamorpholindiyl, thiazolidindiyl, thiochromandiyl, tetrahydroquinolindiyl, and 1,3,5-trithiandiyl. In certain embodiments, the heterocyclylene is optionally substituted with one or more substituents Q as described herein.

The term “halogen”, “halide,” or “halo” refers to fluorine, chlorine, bromine, and/or iodine.

The term “optionally substituted” is intended to mean that a group or substituent, such as an alkyl, alkylene, alkenyl, alkenylene, alkynyl, alkynylene, cycloalkyl, cycloalkylene, aryl, arylene, aralkyl, heteroaryl, heteroarylene, heterocyclyl, or heterocyclylene group, may be substituted with one or more, in one embodiment, one, two, three, or four, substituents Q, each of which is independently selected from, e.g., (a) deuterium (-D), cyano (—CN), halo, nitro (—NO₂), and oxo (═O); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR)NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a}. As used herein, all groups that can be substituted are “optionally substituted.”

In one embodiment, each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{e})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₀ aralkyl, heteroaryl, or heterocyclyl; or (iii) R^{f} and R^{g} together with the N atom to which they are attached form heterocyclyl.

In certain embodiments, “optically active” and “enantiomerically active” refer to a collection of molecules, which has an enantiomeric excess of no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, an optically active compound comprises about 95% or more of one enantiomer and about 5% or less of the other enantiomer based on the total weight of the enantiomeric mixture in question. In certain embodiments, an optically active compound comprises about 98% or more of one enantiomer and about 2% or less of the other enantiomer based on the total weight of the enantiomeric mixture in question. In certain embodiments, an optically active compound comprises about 99% or more of one enantiomer and about 1% or less of the other enantiomer based on the total weight of the enantiomeric mixture in question.

In describing an optically active compound, the prefixes R and S are used to denote the absolute configuration of the compound about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the compound, R and S.

The term “isotopically enriched” refers to a compound that contains an unnatural proportion of an isotope at one or more of the atoms that constitute such a compound. In certain embodiments, an isotopically enriched compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (MS), sulfur-35 (³⁵S), sulfur-36 (MS), chlorine-35 (³⁵Cl), chlorine-36 (⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an isotopically enriched compound is in a stable form, that is, non-radioactive. In certain embodiments, an isotopically enriched compound contains unnatural proportions of one or more isotopes, including, but not limited to, hydrogen (1H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an isotopically enriched compound is in an unstable form, that is, radioactive. In certain embodiments, an isotopically enriched compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, as example, or any carbon can be ¹³C, as example, or any nitrogen can be ¹⁵N, as example, or any oxygen can be ¹⁸O, as example, where feasible according to the judgment of one of ordinary skill in the art.

The term “isotopic enrichment” refers to the percentage of incorporation of a less prevalent isotope (e.g., D for deuterium or hydrogen-2) of an element at a given position in a molecule in the place of a more prevalent isotope (e.g., ¹H for protium or hydrogen-1) of the element. As used herein, when an atom at a particular position in a molecule is designated as a particular less prevalent isotope, it is understood that the abundance of that isotope at that position is substantially greater than its natural abundance.

The term “isotopic enrichment factor” refers the ratio between the isotopic abundance in an isotopically enriched compound and the natural abundance of a specific isotope.

The term “hydrogen” or the symbol “H” refers to the composition of naturally occurring hydrogen isotopes, which include protium (¹H), deuterium (²H or D), and tritium (³H), in their natural abundances. Protium is the most common hydrogen isotope having a natural abundance of more than 99.98%. Deuterium is a less prevalent hydrogen isotope having a natural abundance of about 0.0156%.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156% on average, deuterium enrichment at any position in a compound synthesized using non-enriched starting materials is about 0.0156% on average. As used herein, when a particular position in an isotopically enriched compound is designated as having deuterium, it is understood that the abundance of deuterium at that position in the compound is substantially greater than its natural abundance (0.0156%).

The term “carbon” or the symbol “C” refers to the composition of naturally occurring carbon isotopes, which include carbon-12 (¹²C) and carbon-13 (¹³C) in their natural abundances. Carbon-12 is the most common carbon isotope having a natural abundance of more than 98.89%. Carbon-13 is a less prevalent carbon isotope having a natural abundance of about 1.11%.

The term “carbon-13 enrichment” or “¹³C enrichment” refers to the percentage of incorporation of carbon-13 at a given position in a molecule in the place of carbon. For example, carbon-13 enrichment of 10% at a given position means that 10% of molecules in a given sample contain carbon-13 at the specified position. Because the naturally occurring distribution of carbon-13 is about 1.11% on average, carbon-13 enrichment at any position in a compound synthesized using non-enriched starting materials is about 1.11% on average. As used herein, when a particular position in an isotopically enriched compound is designated as having carbon-13, it is understood that the abundance of carbon-13 at that position in the compound is substantially greater than its natural abundance (1.11%).

The terms “substantially pure” and “substantially homogeneous” mean sufficiently homogeneous to appear free of readily detectable impurities as determined by standard analytical methods used by one of ordinary skill in the art, including, but not limited to, thin layer chromatography (TLC), gel electrophoresis, high performance liquid chromatography (HPLC), gas chromatography (GC), nuclear magnetic resonance (NMR), and mass spectrometry (MS); or sufficiently pure such that further purification would not detectably alter the physical, chemical, biological, and/or pharmacological properties, such as enzymatic and biological activities, of the substance. In certain embodiments, “substantially pure” or “substantially homogeneous” refers to a collection of molecules, wherein at least about 95%, at least about 96%, at least about 97%, at least about 98%, at least about 99%, or at least about 99.5% by weight of the molecules are a single compound, including a single enantiomer, a racemic mixture, or a mixture of enantiomers, as determined by standard analytical methods. As used herein, when an atom at a particular position in an isotopically enriched molecule is designated as a particular less prevalent isotope, a molecule that contains other than the designated isotope at the specified position is an impurity with respect to the isotopically enriched compound. Thus, for a deuterated compound that has an atom at a particular position designated as deuterium, a compound that contains a protium at the same position is an impurity.

The term “solvate” refers to a complex or aggregate formed by one or more molecules of a solute, e.g., a compound provided herein, and one or more molecules of a solvent, which are present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

For a divalent group described herein, no orientation is implied by the direction in which the divalent group is presented. For example, unless a particular orientation is specified, the formula —C(O)NH— represents both —C(O)NH— and —NHC(O)—.

The phrase “an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof” has the same meaning as the phrase “(i) an enantiomer, a mixture of enantiomers, a diastereomer, a mixture of two or more diastereomers, a tautomer, a mixture of two or more tautomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a diastereomer, a mixture of two or more diastereomers, a tautomer, a mixture of two or more tautomers, or an isotopic variant of the compound referenced therein.”

### Compounds

In one embodiment, provided herein is a compound of Formula (I):

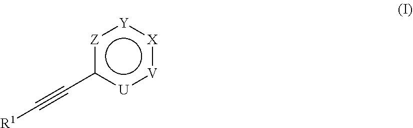

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
U, V, X, and Z are each independently —C(R^{2a})═, —N═, —N(R^{2b})—, —O—, —S—, or -A-L¹-L²-R³; with the proviso that at least one of U and Z is —N═, and one of U, V, X, and Z is -A-L¹-L²_R³;
Y is a bond, —C(R^{2a})═, or —N═;
A is —C(O)—, —C(O)NR^{2a}—, —OC(O)NR^{2a}—, —NR^{1a}C(O)NR^{1d}—, —S(O)—, —S(O)₂—, —S(O)NR^{1a}—, or —S(O)₂NR^{1a}—;
L¹ is a bond, C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
L² is C₆₋₁₄ arylene, heteroarylene, or heterocyclylene;
R¹ is hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₈ aralkyl, heteroaryl, or heterocyclyl;
R³ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2a} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₀ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2b} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; wherein each alkyl, alkylene, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{b}R^{c}(NR^{d})NR^{b}R^{c}, —NR^{b}C(S)R^{d}, —NR^{a}C(S)OR^{d}, —NR^{a}C(S)NR^{b}R^{c}, —NR^{b}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR)NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NR^{e}C(S)NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)—R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In certain embodiments, the compound provided herein is not any one of (4-ethynylthiazol-2-yl)(1H-indol-3-yl)methanone, (4-ethynyl-2-(3-hexylphenyl)-1-methyl-1H-imidazol-5-yl)(4-(pyrrolidin-1-yl)piperidin-1-yl)methanone, (4-ethynyl-1-methyl-2-(3-(trifluoromethyl)phenyl)-1H-imidazol-5-yl)(4-(pyrrolidin-1-yl)piperidin-1-yl)methanone, and (4-ethynyl-1-methyl-2-(3-(trifluoromethoxy)phenyl)-1H-imidazol-5-yl)(4-(pyrrolidin-1-yl)piperidin-1-yl)methanone.

In certain embodiments, the compound provided herein is not any one of 2-ethynyl-N-(1-((1s,4s)-4-hydroxycyclohexyl)-1H-benzo[d]imidazol-2-yl)-6-(1-methyl-1H-pyrazol-4-yl)isonicotinamide, (R)-2-(1-(1-(4-chloro-3-methylbenzyl)piperidin-4-yl)-5-oxopyrrolidine-2-carboxamido)-6-ethynylisonicotinic acid, (6-ethynyl-3-methyl-5-(3-(trifluoromethyl)phenyl)pyridin-2-yl)(4-(pyrrolidin-1-yl)piperidin-1-yl)methanone, and 6-ethynyl-N-(4-fluoro-3-methoxybenzyl)-2-methylpyrimidine-4-carboxamide.

In another embodiment, provided herein is a compound of Formula (II):

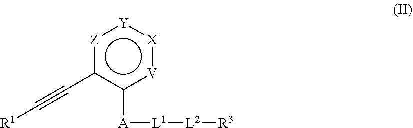

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, A, L¹, L², V, X, and Y are each as defined herein.

In yet another embodiment, provided herein is a compound of Formula (III):

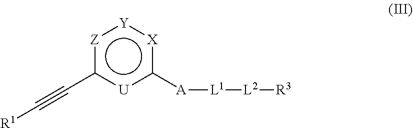

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, A, L¹, L², U, X, Y, and Z are each as defined herein.

In one embodiment, in Formula (I) or (III), U is —N═; X is —C(R^{2a}); Y is a bond; and Z is —O—; wherein R^{2a} is as defined herein. In another embodiment, in Formula (I) or (III), U is —O—; X is —C(R^{2a}); Y is a bond; and Z is —N═; wherein R^{2a} is as defined herein. In yet another embodiment, in Formula (I) or (III), U is —N═; X is —C(R^{2a}); Y is a bond; and Z is —S—; wherein R^{2a} is as defined herein. In yet another embodiment, in Formula (I) or (III), U is —S—; X is —C(R^{2a}); Y is a bond; and Z is —N═; wherein R^{2a} is as defined herein. In yet another embodiment, in Formula (I) or (III), U is —O—; X is —N═; Y is a bond; and Z is —N═. In yet another embodiment, in Formula (I) or (III), U is —S—; X is —N═; Y is a bond; and Z is —N═. In yet another embodiment, in Formula (I) or (III), U is —N═; X is —O—; Y is a bond; and Z is —N═. In yet another embodiment, in Formula (I) or (III), U is —N═; X is —S—; Y is a bond; and Z is —N═. In yet another embodiment, in Formula (I) or (III), U is —N═; X is —N═; Y is a bond; and Z is —O—. In still another embodiment, in Formula (I) or (III), U is —N═; X is —N═; Y is a bond; and Z is —S—.

In one embodiment, in Formula (I) or (III), U is —N═; and X, Y, and Z are each independently —C(R^{2a}); wherein R^{2a} is as defined herein. In another embodiment, in Formula (I) or (III), U, X, and Y are each independently —C(R^{2a}); and Z is —N═; wherein R^{2a} is as defined herein. In yet another embodiment, in Formula (I) or (III). U and X are each —N═; and Y and Z are each independently —C(R^{2a}); wherein R^{2a} is as defined herein. In still another embodiment, in Formula (I) or (III), U and Z are each —N═; and X and Y are each independently —C(R^{2a}); wherein R^{2a} is as defined herein.

In yet another embodiment, provided herein is a compound of Formula (IV):

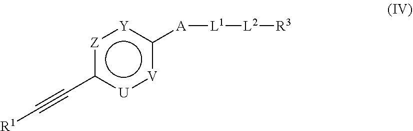

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, A, L¹, L², U, V, Y, and Z are each as defined herein.

In yet another embodiment provided herein is a compound of Formula (V):

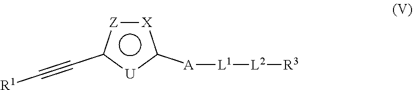

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, A, L¹, L², U, X, and Z are each as defined herein.

In one embodiment, in Formula (V), U is —N═; X is —C(R^{2a}); and Z is —S—; wherein R^{2a} is as defined herein. In another embodiment, in Formula (V), U is —S—; X is —C(R^{2a}); and Z is —N═; wherein R^{2a} is as defined herein. In yet another embodiment, in Formula (V), U is —S—; X is —N═; and Z is —N═. In yet another embodiment, in Formula (V), U is —N═; X is —S—; and Z is —N═. In still another embodiment, in Formula (V), U is —N═; X is —N═; and Z is —S—.

In yet another embodiment, provided herein is a compound of Formula (VI):

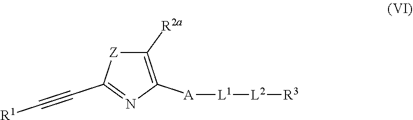

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein Z is —N(R^{2b}) —O—, or —S—; and R¹, R³, R^{2a}, R^{2b}, A, L¹, and L² are each as defined herein.

In yet another embodiment, provided herein is a compound of Formula (VII):

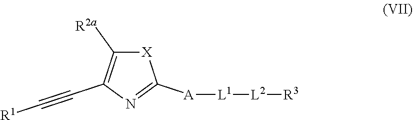

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein X is —N(R^{2b}), —O—, or —S—; and R¹, R³, R^{2a}, R^{2b}, A, L¹, and L² are each as defined herein.

In yet another embodiment, provided herein is a compound of Formula (VIII):

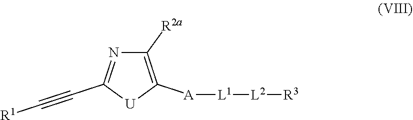

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U is —N(R^{2b})—, —O—, or —S—; and R¹, R³, R^{2a}, R^{2b}, A, L¹, and L² are each as defined herein.

In yet another embodiment, provided herein is a compound of Formula (IX):

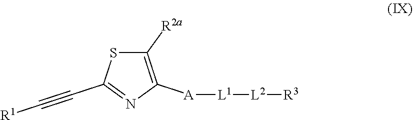

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, A, L¹, and L² are each as defined herein.

In yet another embodiment, provided herein is a compound of Formula (X):

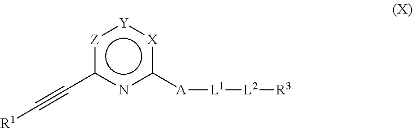

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein X, Y, and Z are each independently —C(R^{2a})═ or —N═; and R¹, R³, R^{2a}, A, L¹, and L² are each as defined herein.

In yet another embodiment, provided herein is a compound of Formula (XI):

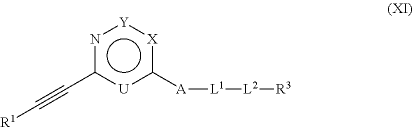

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U, X, and Y are each independently —C(R^{2a})═ or —N═; and R¹, R³, R^{2a}, A, L¹, and L² are each as defined herein.

In yet another embodiment, provided herein is a compound of Formula (XII):

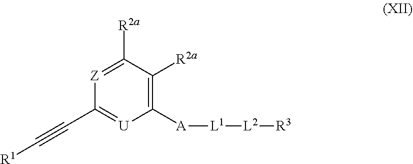

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, A, L¹, and L² are each as defined herein.

In one embodiment, in any one of Formulae (I) to (XII),
A is —C(O)—, —C(O)NR^{1a}—, or —NR^{1a}C(O)NR^{1d}—.
L¹ is a bond, C₁₋₆ alkylene, C₂₋₆ alkenylene, C₃₋₁₀ cycloalkylene, or heterocyclylene;
L² is C₆₋₁₄ arylene, heteroarylene, or heterocyclylene;
R¹ is hydrogen, deuterium, or C₁₋₆ alkyl; and
R³ is (i) hydrogen, deuterium, cyano, halo, or nitro; (ii) C₁₋₆ alkyl, C₆₋₁₄ aryl, heteroaryl, or heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
wherein each alkyl, alkylene, alkenylene, cycloalkylene, aryl, arylene, heteroarylene, heteroaryl, heterocyclyl, and heterocyclylene is optionally substituted with one, two, or three substituents Q; and
wherein R^{1a}, R^{1b}, R^{1c}, and R^{1d} are each as defined herein.

In another embodiment, in any one of Formulae (I) to (XII),
A is —C(O)—, —C(O)NR^{1a}—, or —NR^{1a}C(O)NR^{1d}—;
L¹ is a bond, C₁₋₆ alkylene, C₂₋₆ alkenylene, monocyclic C₃₋₁₀ cycloalkylene, or monocyclic heterocyclylene;
L² is monocyclic or bicyclic C₆₋₁₄ arylene, monocyclic or bicyclic heteroarylene, or monocyclic or bicyclic heterocyclylene;
R¹ is hydrogen, deuterium, or C₁₋₆ alkyl; and
R³ is (i) hydrogen, deuterium, cyano, halo, or nitro; (ii) C₁₋₆ alkyl, monocyclic or bicyclic C₆₋₁₄ aryl, monocyclic, bicyclic, or tricyclic heteroaryl, or bicyclic heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
wherein each alkyl, alkylene, alkenylene, cycloalkylene, aryl, arylene, heteroarylene, heteroaryl, heterocyclyl, and heterocyclylene is optionally substituted with one, two, or three substituents Q;
wherein each substituent Q is independently (i) cyano, halo, or oxo; (ii) C₁₋₆ alkyl or heterocyclyl, each of which is further optionally substituted with one, two, or three substituents Q^{a}; or (iii) —C(O)OR^{a}, —C(O)NR^{b}R^{c}, —OR^{a}, —NR^{b}R^{c}, —S(O)₂R^{a}, or —S(O)₂NR^{b}R^{c}; and
wherein R^{a}, R^{b}, R^{c}, R^{1a}, R^{1b}, R^{1c}, and R^{1d} are each as defined herein.

In yet another embodiment, in any one of Formulae (I) to (XII),
A is —C(O)—, —C(O)NH—, —C(O)N(CH₃)—, or —NHC(O)NH—;
L¹ is a bond; or methanediyl, ethanediyl, ethenediyl, cyclopropanediyl, azetidindiyl, pyrrolidindiyl, piperidindiyl, or piperazindiyl, each of which is optionally substituted with fluoro, hydroxymethyl, hydroxyl, or amino;
L² is phendiyl, 2,3-dihydroindendiyl, naphthdiyl, indoldiyl, indazoldiyl, benzothiazoldiyl, quinoldiyl, quinoldiyl, piperidindiyl, isoindolindiyl, 1,2,3,4-tetrahydroisoquinolindiyl, benzo[d][1,3]dioxoldiyl, or 2,3-dihydrobenzo[b][1,4]dioxindiyl, each of which is optionally substituted with one or two substituents, where each substituent is independently cyano, fluoro, chloro, hydroxyl, or methoxy;
R¹ is hydrogen, deuterium, methyl, or dimethylaminomethyl; and
R³ is (i) hydrogen, deuterium, cyano, chloro, bromo, or nitro; (ii) methyl, ethyl, phenyl, 2,3-dihydroindenyl, pyrazolyl, thiazolyl, pyridinyl, benzo[b]thiophenyl, benzo[d][1,2,3]thiadiazolyl, benzo[d]thiazolyl, imidazo[1,2-a]pyridinyl, imidazo[1,5-a]pyridinyl, indolyl, indazolyl, thiazolo[4,5-c]pyridinyl, [1,2,3]triazolo[1,5-a]pyridinyl, [1,2,4]triazolo[1,5-a]pyridinyl, [1,2,4]triazolo[4,3-a]pyridinyl, isoquinolinyl, quinolinyl, quinazolinyl, quinoxalinyl, 7,8-dihydro-6H-thiazolo[5,4-e]isoindolyl, 2,3-dihydrobenzo[b]-thiophenyl, isoindolinyl, indolinyl, 2,3-dihydroindazolyl, dihydrobenzo[b]thiophenyl, or 3,4-dihydroquinazolinyl, each of which is optionally substituted with one, two, or three substituents, where each substituent is independently cyano, fluoro, oxo, methyl, cyclopropyl, 1-cyanocyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methylsulfonyl, or methylsulfamoyl.

In yet another embodiment, in any one of Formulae (I) to (XII),
A is —C(O)—, —C(O)NH—, —C(O)N(CH₃)—, or —NHC(O)NH—;
L¹ is a bond; or methanediyl, ethane-1,1-diyl, ethane-1,2-diyl, ethene-1,2-diyl, cyclopropane-1,1-diyl, azetidin-1,3-diyl, pyrrolidin-1,2-diyl, piperidin-1,4-diyl, or piperazin-1,4-diyl, each of which is optionally substituted with fluoro, hydroxylmethyl, hydroxyl, or amino;
L² is phen-1,2-diyl, phen-1,3-diyl, phen-1,4-diyl, 2,3-dihydroinden-1,4-diyl, 2,3-dihydroinden-2,5-diyl, naphth-1,5-diyl, naphth-2,6-diyl, indol-2,5-diyl, indazol-3,7-diyl, benzothiazol-2,6-diyl, quinol-2,6-diyl, quinol-3,7-diyl, piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]dioxol-2,5-diyl, or 2,3-dihydrobenzo[b][1,4]dioxin-2,6-diyl, each of which is optionally substituted with one or two substituents, where each substituent is independently cyano, fluoro, chloro, hydroxyl, or methoxy;
R¹ is hydrogen, deuterium, methyl, or dimethylaminomethyl; and
R³ is (i) hydrogen, deuterium, cyano, chloro, bromo, or nitro; (ii) methyl, ethyl, phenyl, 2,3-dihydroinden-4-yl, pyrazol-3-yl, pyrazol-4-yl, thiazol-4-yl, thiazol-5-yl, pyridin-3-yl, benzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, indazol-4-yl, thiazolo[4,5-c]pyridin-7-yl, [1,2,3]triazolo[1,5-a]pyridin-4-yl, [1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, isoquinolin-8-yl, quinolin-5-yl, quinazolin-5-yl, quinoxalin-5-yl, 7,8-dihydro-6H-thiazolo[5,4-e]isoindol-5-yl, 2,3-dihydrobenzo[b]-thiophen-7-yl, isoindolin-4-yl, indolin-4-yl, 2,3-dihydro-1H-indazol-4-yl, 2,3-dihydrobenzo[b]-thiophen-7-yl, or 3,4-dihydroquinazolin-5-yl, each of which is optionally substituted with one, two, or three substituents, where each substituent is independently cyano, fluoro, oxo, methyl, cyclopropyl, 1-cyanocyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methylsulfonyl, or methylsulfamoyl.

In still another embodiment, in any one of Formulae (I) to (XII),
A is —C(O)—, —C(O)NH—, —C(O)N(CH₃)—, or —NHC(O)NH—;
L¹ is a bond, methanediyl, ethane-1,1-diyl, 2-hydroxyethane-1,1-diyl, ethane-1,2-diyl, 1-hydroxyethane-1,2-diyl, 1-aminoethane-1,2-diyl, ethene-1,2-diyl, cyclopropan-1,1-diyl, azetidin-1,3-diyl, pyrrolidin-1,2-diyl, piperidin-1,4-diyl, 4-fluropiperidin-1,4-diyl, 4-hydroxypiperidin-1,4-diyl, piperazin-1,4-diyl, or 2-hydroxymethylpiperazin-1,4-diyl;
L² is phen-1,2-diyl, phen-1,3-diyl, phen-1,4-diyl, 4-methoxyphen-1,3-diyl, 2-cyanophen-1,4-diyl, 2-fluorophen-1,4-diyl, 2-chlorophen-1,4-diyl, 2-hydroxyphen-1,4-diyl, 2,3-dihydroinden-1,4-diyl, 2,3-dihydroinden-2,5-diyl, naphth-1,5-diyl, naphth-2,6-diyl, pyrazol-1,3-diyl, pyrazol-1,4-diyl, pyridin-2,3-diyl, pyridin-2,5-diyl, indol-2,5-diyl, indazol-3,7-diyl, benzothiazol-2,6-diyl, quinol-2,6-diyl, quinol-3,7-diyl, piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]-dioxol-2,5-diyl, or 2,3-dihydrobenzo[b][1,4]dioxin-2,6-diyl;
R¹ is hydrogen, deuterium, methyl, or dimethylaminomethyl; and
R³ is (i) hydrogen, deuterium, cyano, chloro, bromo, or nitro; (ii) methyl, methoxycarbonylmethyl, carbamoylmethyl, hydroxymethyl, 2-methoxycarbonylethyl, 2-hydroxylethyl, phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(azetidin-1-yl)phenyl, 3-(pyrrolidin-1-yl)phenyl, 3-(3-hydroxypyrrolidin-1-yl)phenyl, 3-(2-oxopyrrolidin-1-yl)phenyl, 3-(2-oxoimidazolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(3-hydroxyazetidin-1-yl)phenyl, 3-carbamoylphenyl, 2-methylcarbamoylphenyl, 3-methylcarbamoylphenyl, 2-methylsulfamoylphenyl, 2-dimethylsulfamoylphenyl, 2-methyl-sulfonylphenyl, 3-oxo-2,3-dihydro-1H-inden-4-yl, pyrazol-3-yl, pyrazol-4-yl, 1-methylpyrazol-3-yl, 1-methylpyrazol-4-yl, thiazol-4-yl, thiazol-5-yl, pyridin-3-yl, 1,1-dioxidobenzo[b]-thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, 6-fluorobenzo[d]thiazol-5-yl, 6-cyanobenzo[d]thiazol-7-yl, 6-fluorobenzo[d]thiazol-7-yl, 5-methoxycarbonylbenzo[d]-thiazol-7-yl, 6-methoxycarbonylbenzo[d]thiazol-7-yl, 5-carbamoylbenzo[d]thiazol-7-yl, 6-carbamoylbenzo[d]thiazol-7-yl, 5-methylcarbamoylbenzo[d]thiazol-7-yl, 6-methylcarbamoyl-benzo[d]thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethyl-indazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, [1,2,4]triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxy-quinazolin-5-yl, quinoxalin-5-yl, 8-oxo-7,8-dihydro-6H-thiazolo[5,4-e]isoindol-5-yl, 3-hydroxy-1,1-dioxido-2,3-dihydrobenzo[b]thiophen-7-yl, 1,1-dioxido-3-oxo-2,3-dihydrobenzo[b]-thiophen-7-yl, 1-oxoisoindolin-4-yl, 3-oxoisoindolin-4-yl, 2-methyl-1-oxoisoindolin-4-yl, 2,3-dioxoindolin-4-yl, 2-oxoindolin-4-yl, 1-methyl-2-oxoindolin-4-yl, 1-methyl-3-oxo-2,3-dihydro-1H-indazol-4-yl, 2,2-difluoro-1,1-dioxido-3-oxo-2,3-dihydrobenzo[b]-thiophen-7-yl, or 3-methyl-4-oxo-3,4-dihydroquinazolin-5-yl; or (iii) methoxycarbonyl, hydroxyl, methoxy, amino, acetamido, methylsulfonamido, methylsulfonyl, or methylsulfamoyl.

In yet another embodiment, provided herein is a compound of Formula (XIII):

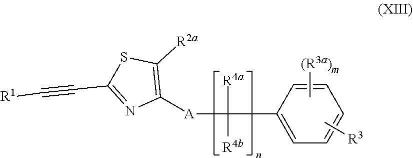

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R^{3a} is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{4a} and R^{4b} is independently (i) hydrogen, deuterium, cyano, halo, or nitro; (b) C₁₋₆ alkyl, C₂₋₆ alkenyl, C₂₋₆ alkynyl, C₁₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or R^{4a} and R^{4b} together with the carbon atom to which they are attached form C₃₋₁₀ cycloalkyl;
m is an integer of 0, 1, 2, 3, or 4;
n is an integer of 0, 1, 2, 3, 4, 5, or 6; and
R¹, R³, R^{1a}, R^{1b}, R^{1c}, R^{1d}, R^{2a}, and A are each as defined herein;
wherein each alkyl, alkenyl, alkynyl, cycloalkyl, aryl, aralkyl, heteroaryl, and heterocyclyl is optionally substituted with one or more, in one embodiment, one, two, three, or four, substituents Q.

In yet another embodiment, provided herein is a compound of Formula (XIV):

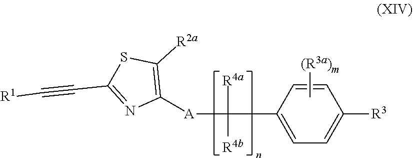

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, A, m, and n are each as defined herein.

In yet another embodiment, provided herein is a compound of Formula (XV):

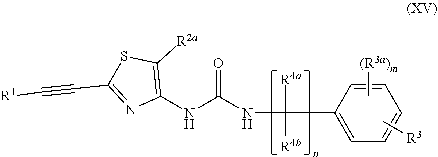

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, m, and n are each as defined herein.

In yet another embodiment, provided herein is a compound of Formula (XVI):

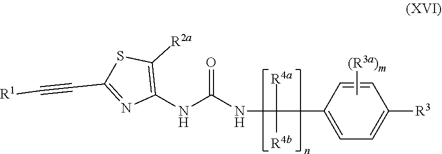

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, m, and n are each as defined herein.

In yet another embodiment, provided herein is a compound of Formula (XVII):

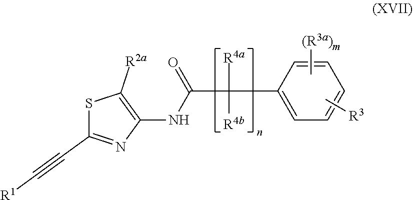

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, and n are each as defined herein.

In yet another embodiment, provided herein is a compound of Formula (XVIII):

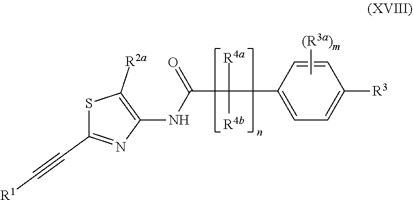

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, m, and n are each as defined herein.

In yet another embodiment, provided herein is a compound of Formula (XIX):

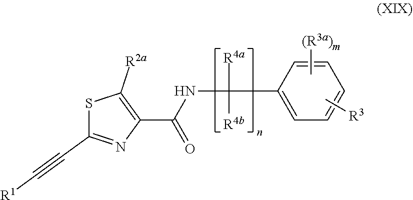

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, m, and n are each as defined herein.

In yet another embodiment, provided herein is a compound of Formula (XX):

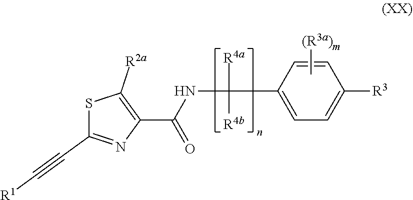

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R^{2a}, R^{3a}, R^{4a}, R^{4b}, m, and n are each as defined herein.

In yet another embodiment, provided herein is a compound of Formula (XXI):

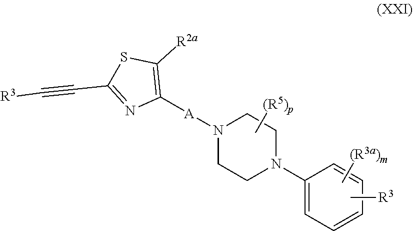

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R⁵ is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
p is an integer of 0, 1, 2, 3, or 4; and
R¹, R³, R^{1a}, R^{1b}, R^{1c}, R^{1d}, R^{2a}, R^{3a}, A, and m are each as defined herein.

In yet another embodiment, provided herein is a compound of Formula (XXII):

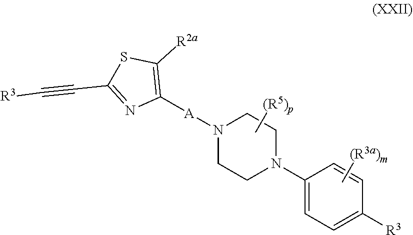

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁵, R^{2a}, R^{3a}, A, m, and p are each as defined herein.

In yet another embodiment, provided herein is a compound of Formula (XXIII):

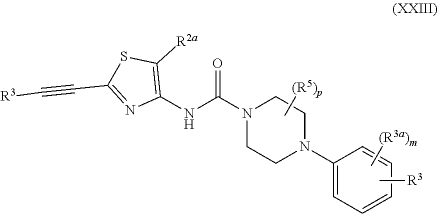

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁵, R^{2a}, R^{3a}, m, and p are each as defined herein.

In yet another embodiment, provided herein is a compound of Formula (XXIV):

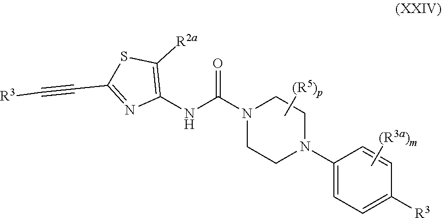

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁵, R^{2a}, R^{3a}, m, and p are each as defined herein.

In yet another embodiment, provided herein is a compound of Formula (XXV):

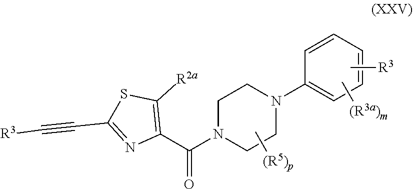

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁵, R^{2a}, R^{3a}, m, and p are each as defined herein.

In yet another embodiment, provided herein is a compound of Formula (XXVI):

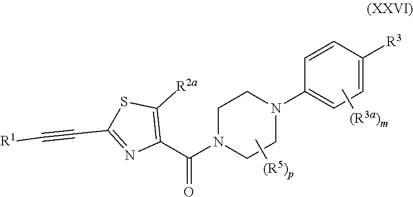

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R¹, R³, R⁵, R^{2a}, R^{3a}, m, and p are each as defined herein.

In yet another embodiment, provided herein is a compound of Formula (XXVII):

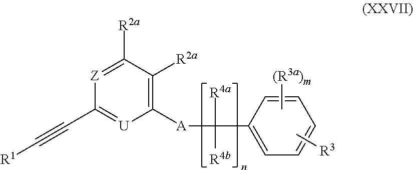

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, A, m, and n are each as defined herein.

In yet another embodiment, provided herein is a compound of Formula (XXVIII):

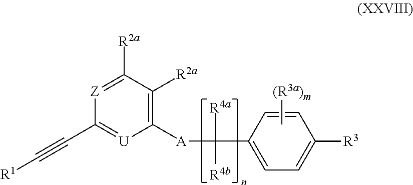

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, A, m, and n are each as defined herein.

In yet another embodiment, provided herein is a compound of Formula (XXIX):

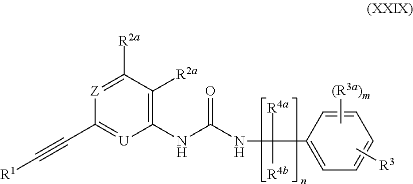

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXX):

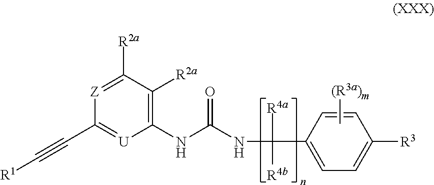

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXXI):

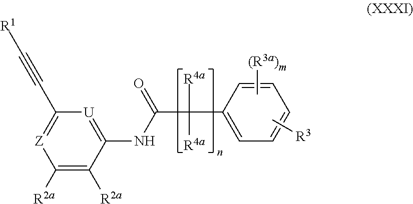

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXXII):

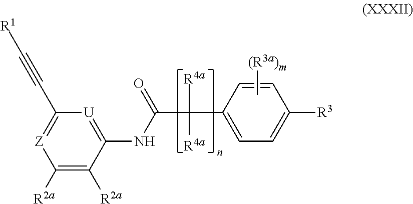

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXXIII):

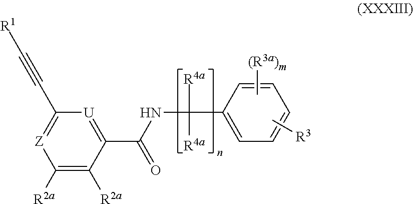

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXXIV):

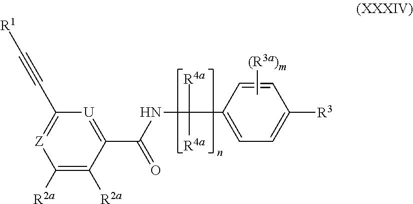

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R^{2a}, R^{3a}, R^{4a}, R^{4b}, m, and n are each as defined herein.

In yet another embodiment, provided herein is a compound of Formula (XXXV):

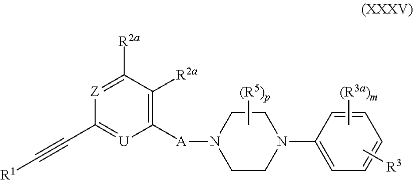

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R⁵, R^{2a}, R^{3a}, A, m, and p are each as defined herein.

In yet another embodiment, provided herein is a compound of Formula (XXXVI):

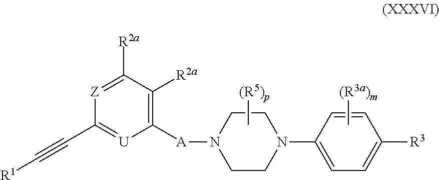

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R⁵, R^{2a}, R^{3a}, A, m, and p are each as defined herein.

In yet another embodiment, provided herein is a compound of Formula (XXXVII):

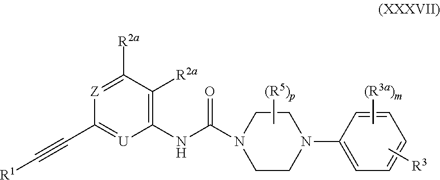

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R⁵, R^{2a}, R^{3a}, m, and p are each as defined herein.

In yet another embodiment, provided herein is a compound of Formula (XXXVIII):

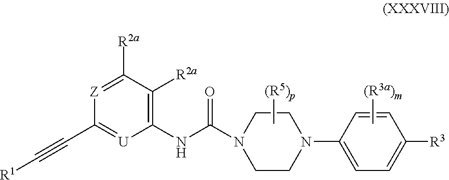

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2′})═ or —N═; and R¹, R³, R⁵, R^{2a}, R^{3a}, m, and p are each as defined herein.

In yet another embodiment, provided herein is a compound of Formula (XXXIX):

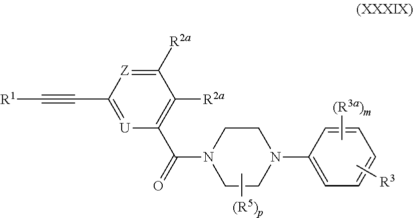

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R⁵, R^{2a}, R^{3a}, m, and p are each as defined herein.

In still another embodiment, provided herein is a compound of Formula (XL):

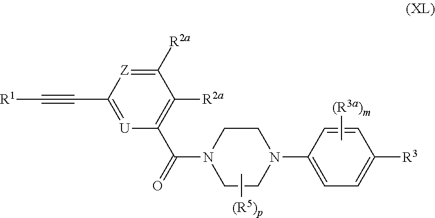

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U and Z are each independently —C(R^{2a})═ or —N═; and R¹, R³, R⁵, R^{2a}, R^{3a}, m, and p are each as defined herein.

In one embodiment, in any one of Formulae (XXVII) to (XL), U and Z are each —N═. In another embodiment, in any one of Formulae (XXVII) to (XL), U is —N═ and Z is —C(R^{2a})═. In yet another embodiment, in any one of Formulae (XXVII) to (XL), U is —C(R^{2a})═ and Z is —N═.

In one embodiment, in any one of Formulae (XIII) to (XX) and (XXVII) to (XXXIV),
A, if present, is —C(O)—, —CO(O)NR^{1a}—, or —NR^{1a}C(O)NR^{1d}—;
R¹ is hydrogen, deuterium, or C₁₋₆ alkyl;
R³ is (i) hydrogen, deuterium, cyano, halo, or nitro; (ii) C₁₋₆ alkyl, C₆₋₁₄ aryl, heteroaryl, or heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
each R^{2a} is independently hydrogen, C₁₋₆ alkyl, or C₁₋₁₄ aryl;
each R^{3a} is independently cyano or halo;
each R^{4a} is independently hydrogen, C₁₋₆ alkyl, or —NR^{1b}R^{1c}; and each R^{4b} is hydrogen; or R^{4s} and R^{4b} together with the carbon atom to which they are attached form C₃₋₁₀ cycloalkylene;
m is an integer of 0, 1, or 2; and
n is an integer of 0, 1, 2, 3, or 4;
wherein each alkyl, aryl, heteroaryl, and heterocyclyl is optionally substituted with one, two, or three substituents Q; and
wherein R^{1a}, R^{1b}, R^{1c}, and R^{1d} are each as defined herein.

In another embodiment, in any one of Formulae (XIII) to (XX) and (XXVII) to (XXXIV),
A, if present, is —C(O)—, —C(O)NR^{1a}—, or —NR^{1a}C(O)NR^{1d}—;
R¹ is hydrogen, deuterium, or C₁₋₆ alkyl;
R³ is (i) hydrogen, deuterium, cyano, halo, or nitro; (ii) C₁₋₆ alkyl, monocyclic or bicyclic C₆₋₁₄ aryl, monocyclic, bicyclic, or tricyclic heteroaryl, or bicyclic heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
each R^{2a} is independently hydrogen, C₁₋₆ alkyl, or monocyclic or bicyclic C₆₋₁₄ aryl;
each R^{3a} is independently cyano or halo;
each R^{4a} is independently hydrogen, C₁₋₆ alkyl, or —NR^{1b}R^{1c}; and each R^{4b} is hydrogen; or R^{4a} and R^{4b} together with the carbon atom to which they are attached form monocyclic C₃₋₁₀ cycloalkylene;
m is an integer of 0, 1, or 2; and
n is an integer of 0, 1, 2, 3, or 4;
wherein each alkyl, aryl, heteroaryl, and heterocyclyl is optionally substituted with one, two, or three substituents Q;
wherein each substituent Q is independently (i) cyano, halo, or oxo; (ii) C₁₋₆ alkyl or heterocyclyl, each of which is further optionally substituted with one, two, or three substituents Q^{a}; or (iii) —C(O)OR^{a}, —C(O)NR^{b}R^{c}, —OR^{a}, —NR^{b}R^{c}, —S(O)₂R^{a}, or —S(O)₂NR^{b}R^{c}; and wherein R^{a}, R^{b}, R^{c}, R^{1a}, R^{1b}, R^{1c}, and R^{1d} are each as defined herein.

In yet another embodiment, in any one of Formulae (XIII) to (XX) and (XXVII) to (XXXIV),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, ethyl, phenyl, 2,3-dihydroindenyl, pyrazolyl, thiazolyl, pyridinyl, benzo[b]thiophenyl, benzo[d][1,2,3]-thiadiazolyl, benzo[d]thiazolyl, imidazo[1,2-a]pyridinyl, imidazo[1,5-a]pyridinyl, indolyl, indazolyl, thiazolo[4,5-c]pyridinyl, [1,2,3]triazolo[1,5-a]pyridinyl, [1,2,4]triazolo[1,5-a]-pyridinyl, [1,2,4]triazolo[4,3-a]pyridinyl, isoquinolinyl, quinolinyl, quinazolinyl, quinoxalinyl, 7,8-dihydro-6H-thiazolo[5,4-e]isoindolyl, 2,3-dihydrobenzo[b]-thiophenyl, isoindolinyl, indolinyl, 2,3-dihydroindazolyl, dihydrobenzo[b]thiophenyl, or 3,4-dihydroquinazolinyl, each of which is optionally substituted with one, two, or three substituents, where each substituent is independently cyano, fluoro, oxo, methyl, cyclopropyl, 1-cyanocyclopropyl, 1-hydroxy-cyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methylsulfonyl, or methylsulfamoyl;
each R^{2a} is independently hydrogen, methyl, or phenyl;
each R^{3a} is independently cyano, fluoro, or chloro;
each R^{4a} is independently hydrogen, methyl, or amino; and each R^{4b} is hydrogen;
or R^{4a} and R^{4b} together with the carbon atom to which they are attached form cyclopropanediyl;
m is an integer of 0 or 1; and
n is an integer of 0, 1, 2, or 3.

In yet another embodiment, in any one of Formulae (XIII) to (XX) and (XXVII) to (XXXIV),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, ethyl, phenyl, 2,3-dihydroinden-4-yl, pyrazol-3-yl, pyrazol-4-yl, thiazol-4-yl, thiazol-5-yl, pyridin-3-yl, benzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, imidazo[1,2-a]pyridin-5-yl, imidazo[1,2-a]-pyridin-8-yl, imidazo[1,5-a]pyridin-5-yl, imidazo[1,5-a]pyridin-8-yl, indol-4-yl, indazol-4-yl, thiazolo[4,5-c]pyridin-7-yl, [1,2,3]triazolo[1,5-a]pyridin-4-yl, [1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, isoquinolin-8-yl, quinolin-5-yl, quinazolin-5-yl, quinoxalin-5-yl, 7,8-dihydro-6H-thiazolo[5,4-e]isoindol-5-yl, 2,3-dihydrobenzo[b]-thiophen-7-yl, isoindolin-4-yl, indolin-4-yl, 2,3-dihydro-1H-indazol-4-yl, 2,3-dihydrobenzo[b]-thiophen-7-yl, or 3,4-dihydro-quinazolin-5-yl, each of which is optionally substituted with one, two, or three substituents, where each substituent is independently cyano, fluoro, oxo, methyl, cyclopropyl, 1-cyano-cyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxy-cyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methyl-sulfonyl, or methylsulfamoyl;
each R^{2a} is independently hydrogen, methyl, or phenyl;
each R^{3a} is independently cyano, fluoro, or chloro;
each R^{4a} is independently hydrogen, methyl, or amino; and each R^{4b} is hydrogen;
or R^{4a} and R^{4b} together with the carbon atom to which they are attached form cyclopropanediyl;
m is an integer of 0 or 1; and
n is an integer of 0, 1, or 2.

In still another embodiment, in any one of Formulae (XIII) to (XX) and (XXVII) to (XXXIV),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, methoxycarbonylmethyl, carbamoylmethyl, hydroxymethyl, 2-methoxycarbonylethyl, 2-hydroxylethyl, phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(azetidin-1-yl)phenyl, 3-(pyrrolidin-1-yl)phenyl, 3-(3-hydroxypyrrolidin-1-yl)phenyl, 3-(2-oxopyrrolidin-1-yl)phenyl, 3-(2-oxoimidazolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(3-hydroxyazetidin-1-yl)phenyl, 3-carbamoyl-phenyl, 2-methylcarbamoylphenyl, 3-methylcarbamoylphenyl, 2-methylsulfamoylphenyl, 2-dimethylsulfamoylphenyl, 2-methyl-sulfonylphenyl, 3-oxo-2,3-dihydro-1H-inden-4-yl, pyrazol-3-yl, pyrazol-4-yl, 1-methylpyrazol-3-yl, 1-methylpyrazol-4-yl, thiazol-4-yl, thiazol-5-yl, pyridin-3-yl, 1,1-dioxidobenzo[b]-thiophen-7-yl, benzo[d][1,2,3]thiadiazol-7-yl, benzo[d]-thiazol-4-yl, benzo[d]thiazol-5-yl, benzo[d]thiazol-6-yl, benzo[d]thiazol-7-yl, 6-fluorobenzo[d]-thiazol-5-yl, 6-cyanobenzo[d]thiazol-7-yl, 6-fluorobenzo[d]thiazol-7-yl, 5-methoxycarbonyl-benzo[d]-thiazol-7-yl, 6-methoxycarbonylbenzo[d]thiazol-7-yl, 5-carbamoylbenzo[d]thiazol-7-yl, 6-carbamoylbenzo[d]thiazol-7-yl, 5-methylcarbamoylbenzo[d]thiazol-7-yl, 6-methyl-carbamoylbenzo[d]thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethyl-indazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, [1,2,4]triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxy-quinazolin-5-yl, quinoxalin-5-yl, 8-oxo-7,8-dihydro-6H-thiazolo[5,4-e]isoindol-5-yl, 3-hydroxy-1,1-dioxido-2,3-dihydrobenzo[b]thiophen-7-yl, 1,1-dioxido-3-oxo-2,3-dihydrobenzo[b]-thiophen-7-yl, 1-oxo-isoindolin-4-yl, 3-oxoisoindolin-4-yl, 2-methyl-1-oxoisoindolin-4-yl, 2,3-dioxoindolin-4-yl, 2-oxoindolin-4-yl, 1-methyl-2-oxoindolin-4-yl, 1-methyl-3-oxo-2,3-dihydro-1H-indazol-4-yl, 2,2-difluoro-1,1-dioxido-3-oxo-2,3-dihydrobenzo[b]-thiophen-7-yl, or 3-methyl-4-oxo-3,4-dihydro-quinazolin-5-yl; or (iii) methoxycarbonyl, hydroxyl, methoxy, amino, acetamido, methyl-sulfonamido, methylsulfonyl, or methylsulfamoyl;
each R^{2a} is independently hydrogen, methyl, or phenyl;
each R^{3a} is independently cyano, fluoro, or chloro;
each R^{4a} is independently hydrogen, methyl, or amino; and each R^{4b} is hydrogen;
or R^{4a} and R^{4b} together with the carbon atom to which they are attached form cyclopropanediyl;
m is an integer of 0 or 1; and
n is an integer of 1 or 2.

In one embodiment, in any one of Formulae (XXI) to (XXVI) and (XXXV) to (XL),
A, if present, is —C(O)—, —C(O)NR^{1a}, or —NR^{1a}C(O)NR^{1d}—;
R¹ is hydrogen, deuterium, or C₁₋₆ alkyl;
R³ is (i) hydrogen, deuterium, cyano, halo, or nitro; (ii) C₁₋₆ alkyl, C₆₋₁₄ aryl, heteroaryl, or heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
R⁵ is hydrogen, deuterium, halo, C₁₋₆ alkyl, or heterocyclyl;
each R^{2a} is independently hydrogen, C₁₋₆ alkyl, or C₆₋₁₄ aryl;
each R^{3a} is independently cyano or halo;
n is an integer of 0, 1, 2, 3, or 4; and
p is an integer of 0, 1, 3, or 4;
wherein each alkyl, aryl, heteroaryl, and heterocyclyl is optionally substituted with one, two, or three substituents Q; and
wherein R^{1a}, R^{1b}, R^{1c}, and R^{1d} are each as defined herein.

In another embodiment, in any one of Formulae (XXI) to (XXVI) and (XXXV) to (XL),
A, if present, is —C(O)—, —C(O)NR^{1a}—, or —NR^{1a}C(O)NR^{1d}—;
R¹ is hydrogen, deuterium, or C₁₋₆ alkyl;
R³ is (i) hydrogen, deuterium, cyano, halo, or nitro; (ii) C₁₋₆ alkyl, monocyclic or bicyclic C₆₋₁₄ aryl, monocyclic, bicyclic, or tricyclic heteroaryl, or bicyclic heterocyclyl; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c};
R⁵ is hydrogen, deuterium, halo, or C₁₋₆ alkyl;
each R^{2a} is independently hydrogen, C₁₋₆ alkyl, or monocyclic or bicyclic C₆₋₁₄ aryl;
each R^{3a} is independently cyano or halo;
n is an integer of 0, 1, 2, 3, or 4; and
p is an integer of 0, 1, or 2;
wherein each alkyl, aryl, heteroaryl, and heterocyclyl is optionally substituted with one, two, or three substituents Q;
wherein each substituent Q is independently (i) cyano, halo, or oxo; (ii) C₁_alkyl or heterocyclyl, each of which is further optionally substituted with one, two, or three substituents Q^{a}; or (iii) —C(O)OR^{a}, —C(O)NR^{b}R^{c}, —OR^{a}, —NR^{b}R^{c}, —S(O)₂R^{a}, or —S(O)₂NR^{b}R^{c}; and
wherein R^{a}, R^{b}, R^{c}, R^{1a}, R^{1b}, R^{1c}, and R^{1d} are each as defined herein.

In yet another embodiment, in any one of Formulae (XXI) to (XXVI) and (XXXV) to (XL),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, ethyl, phenyl, 2,3-dihydroindenyl, pyrazolyl, thiazolyl, pyridinyl, benzo[b]thiophenyl, benzo[d][1,2,3]-thiadiazolyl, benzo[d]thiazolyl, imidazo[1,2-a]pyridinyl, imidazo[1,5-a]pyridinyl, indolyl, indazolyl, thiazolo[4,5-c]pyridinyl, [1,2,3]triazolo[1,5-a]pyridinyl, [1,2,4]triazolo[1,5-a]-pyridinyl, [1,2,4]triazolo[4,3-a]pyridinyl, isoquinolinyl, quinolinyl, quinazolinyl, quinoxalinyl, 7,8-dihydro-6H-thiazolo[5,4-e]isoindolyl, 2,3-dihydrobenzo[b]-thiophenyl, isoindolinyl, indolinyl, 2,3-dihydroindazolyl, dihydrobenzo[b]thiophenyl, or 3,4-dihydroquinazolinyl, each of which is optionally substituted with one, two, or three substituents, where each substituent is independently cyano, fluoro, oxo, methyl, cyclopropyl, 1-cyanocyclopropyl, 1-hydroxy-cyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methylsulfonyl, or methylsulfamoyl;
R⁵ is hydroxymethyl;
each R^{2a} is independently hydrogen, methyl, or phenyl;
each R^{3a} is independently cyano, fluoro, or chloro;
n is an integer of 0, 1, 2, or 3; and
p is an integer of 0 or 1.

In yet another embodiment, in any one of Formulae (XXI) to (XXVI) and (XXXV) to (XL),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, ethyl, phenyl, 2,3-dihydroinden-4-yl, pyrazol-3-yl, pyrazol-4-yl, thiazol-4-yl, thiazol-5-yl, pyridin-3-yl, benzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, imidazo[1,2-a]pyridin-5-yl, imidazo[1,2-a]-pyridin-8-yl, imidazo[1,5-a]pyridin-5-yl, imidazo[1,5-a]pyridin-8-yl, indol-4-yl, indazol-4-yl, thiazolo[4,5-c]pyridin-7-yl, [1,2,3]triazolo[1,5-a]pyridin-4-yl, [1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]-pyridin-8-yl, isoquinolin-5-yl, isoquinolin-8-yl, quinolin-5-yl, quinazolin-5-yl, quinoxalin-5-yl, 7,8-dihydro-6H-thiazolo[5,4-e]isoindol-5-yl, 2,3-dihydrobenzo[b]-thiophen-7-yl, isoindolin-4-yl, indolin-4-yl, 2,3-dihydro-1H-indazol-4-yl, 2,3-dihydrobenzo[b]-thiophen-7-yl, or 3,4-dihydro-quinazolin-5-yl, each of which is optionally substituted with one, two, or three substituents, where each substituent is independently cyano, fluoro, oxo, methyl, cyclopropyl, 1-cyano-cyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxo-oxazolidin-3-yl, methoxycarbonyl, carbamoyl, methylcarbamoyl, hydroxyl, (3-hydroxy-cyclobutyl)amino, oxetan-3-ylamino, methylsulfonyl, methylsulfamoyl, or dimethylsulfamoyl; or (iii) methoxycarbonyl, hydroxyl, methoxy, amino, acetamido, methylsulfonamido, methyl-sulfonyl, or methylsulfamoyl;
R⁵ is hydroxymethyl;
each R^{2a} is independently hydrogen, methyl, or phenyl;
each R³, is independently cyano, fluoro, or chloro;
n is an integer of 0, 1, or 2; and
p is an integer of 0 or 1.

In still another embodiment, in any one of Formulae (XXI) to (XXVI) and (XXXV) to (XL),
A, if present, is —C(O)—, —C(O)NH—, —C(O)N(CH₃)—, or —NHC(O)NH—;
R¹ is hydrogen, deuterium, methyl, or dimethylaminomethyl;
R³ is (i) hydrogen, deuterium, cyano, chloro, bromo, or nitro; (ii) methyl, methoxycarbonylmethyl, carbamoylmethyl, hydroxymethyl, 2-methoxycarbonylethyl, 2-hydroxylethyl, phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(azetidin-1-yl)phenyl, 3-(pyrrolidin-1-yl)phenyl, 3-(3-hydroxypyrrolidin-1-yl)phenyl, 3-(2-oxopyrrolidin-1-yl)phenyl, 3-(2-oxoimidazolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(3-hydroxyazetidin-1-yl)phenyl, 3-carbamoyl-phenyl, 2-methylcarbamoylphenyl, 3-methylcarbamoylphenyl, 2-methylsulfamoylphenyl, 2-dimethylsulfamoylphenyl, 2-methyl-sulfonylphenyl, 3-oxo-2,3-dihydro-1H-inden-4-yl, pyrazol-3-yl, pyrazol-4-yl, 1-methylpyrazol-3-yl, 1-methylpyrazol-4-yl, thiazol-4-yl, thiazol-5-yl, pyridin-3-yl, 1,1-dioxidobenzo[b]-thiophen-7-yl, benzo[d][1,2,3]thiadiazol-7-yl, benzo[d]-thiazol-4-yl, benzo[d]thiazol-5-yl, benzo[d]thiazol-6-yl, benzo[d]thiazol-7-yl, 6-fluorobenzo[d]-thiazol-5-yl, 6-cyanobenzo[d]thiazol-7-yl, 6-fluorobenzo[d]thiazol-7-yl, 5-methoxycarbonyl-benzo[d]-thiazol-7-yl, 6-methoxycarbonylbenzo[d]thiazol-7-yl, 5-carbamoylbenzo[d]thiazol-7-yl, 6-carbamoylbenzo[d]thiazol-7-yl, 5-methylcarbamoylbenzo[d]thiazol-7-yl, 6-methyl-carbamoylbenzo[d]thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethyl-indazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, [1,2,4]triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxy-quinazolin-5-yl, quinoxalin-5-yl, 8-oxo-7,8-dihydro-6H-thiazolo[5,4-e]isoindol-5-yl, 3-hydroxy-1,1-dioxido-2,3-dihydrobenzo[b]thiophen-7-yl, 1,1-dioxido-3-oxo-2,3-dihydrobenzo[b]-thiophen-7-yl, 1-oxo-isoindolin-4-yl, 3-oxoisoindolin-4-yl, 2-methyl-1-oxoisoindolin-4-yl, 2,3-dioxoindolin-4-yl, 2-oxoindolin-4-yl, 1-methyl-2-oxoindolin-4-yl, 1-methyl-3-oxo-2,3-dihydro-1H-indazol-4-yl, 2,2-difluoro-1,1-dioxido-3-oxo-2,3-dihydrobenzo[b]-thiophen-7-yl, or 3-methyl-4-oxo-3,4-dihydro-quinazolin-5-yl; or (iii) methoxycarbonyl, hydroxyl, methoxy, amino, acetamido, methyl-sulfonamido, methylsulfonyl, or methylsulfamoyl;
R⁵ is hydroxymethyl;
each R^{2a} is independently hydrogen, methyl, or phenyl;
each R^{3a} is independently cyano, fluoro, or chloro;
n is an integer of 1 or 2; and
p is an integer of 0 or 1.

In one embodiment, provided herein is a compound of Formula (XLI):

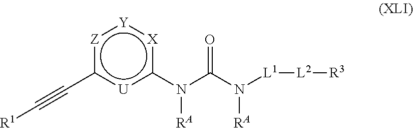

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein each R^{A} is independently hydrogen or C₁₋₆ alkyl, optionally substituted with one ore substituents Q; and R¹, R³, L¹, L², U, X, Y, and Z are each as defined herein.

In one embodiment, in Formula (XLI), U is —N═; X is —C(R^{2a}); Y is a bond; and Z is —O—; wherein R^{2a} is as defined herein. In another embodiment, in Formula (XLI), U is —O—; X is —C(R^{2a}); Y is a bond; and Z is —N═; wherein R^{2b} is as defined herein. In yet another embodiment, in Formula (XLI), U is —N═; X is —C(R^{2b}); Y is a bond; and Z is —S—; wherein R^{2a} is as defined herein. In yet another embodiment, in Formula (XLI), U is —S—; X is —C(R^{2a}); Y is a bond; and Z is —N═; wherein R^{2b} is as defined herein. In yet another embodiment, in Formula (XLI), U is —O—; X is —N═; Y is a bond; and Z is —N═. In yet another embodiment, in Formula (XLI), U is —S—; X is —N═; Y is a bond; and Z is —N═. In yet another embodiment, in Formula (XLI), U is —N═; X is —O—; Y is a bond; and Z is —N═. In yet another embodiment, in Formula (XLI), U is —N═; X is —S—; Y is a bond; and Z is —N═. In yet another embodiment, in Formula (XLI), U is —N═; X is —N═; Y is a bond; and Z is —O—. In still another embodiment, in Formula (XLI), U is —N═; X is —N═; Y is a bond; and Z is —S—.

In one embodiment, in Formula (XLI), U is —N═; and X, Y, and Z are each independently —C(R^{2a}); wherein R^{2a} is as defined herein. In another embodiment, in Formula (XLI), U, X, and Y are each independently —C(R^{2a}); and Z is —N═; wherein R^{2a} is as defined herein. In yet another embodiment, in Formula (XLI), U and X are each —N═; and Y and Z are each independently —C(R^{2a}); wherein R^{2a} is as defined herein. In still another embodiment, in Formula (XLI), U and Z are each —N═; and X and Y are each independently —C(R^{2a}); wherein R^{2a} is as defined herein.

In another embodiment, provided herein is a compound of Formula (XLII):

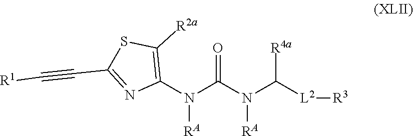

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein R^{4a} is C₁₋₆ alkyl, optionally substituted with one ore substituents Q, or —C(O)NR^{1b}R^{1c}; and R¹, R³, R^{1b}, R^{1c}, R^{2a}, R^{4a}, R^{A}, and L² are each as defined herein.

In yet another embodiment provided herein is a compound of Formula (XLIII):

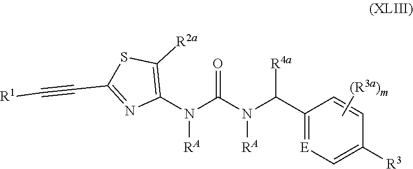

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein E is —C(H)═ or —N═; R^{4a} is C₁₋₆ alkyl, optionally substituted with one ore substituents Q, or —C(O)NR^{1b}R^{1c}; and R¹, R³, R^{1b}, R^{1c}, R^{2a}, R^{3a}, R^{4a}, and m are each as defined herein.

In yet another embodiment, provided herein is a compound of Formula (XLIV):

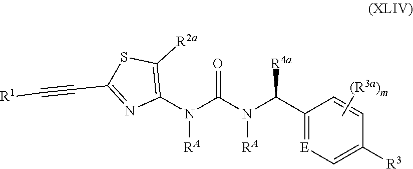

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein E is —C(H)═ or —N═; R^{4a} is C₁₋₆ alkyl, optionally substituted with one ore substituents Q. or —C(O)NR^{1b}R^{1c}; and R¹, R³, R^{1b}, R^{1c}, R^{2a}, R^{3a}, R^{4a}, and m are each as defined herein.

In still another embodiment, provided herein is a compound of Formula (XLV):

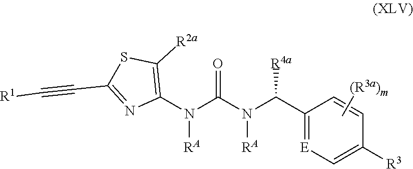

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein E is —C(H)═ or —N═; R^{4a} is C₁₋₆ alkyl, optionally substituted with one ore substituents Q, or —C(O)NR^{1b}R^{1c}; and R¹, R³, R^{1b}, R^{1c}, R^{2a}, R^{3a}, R^{4a}, and m are each as defined herein.

In certain embodiments, in any one of Formulae (XLII) to (XLV), R^{4a} is C₁₋₆ alkyl, optionally substituted with one ore substituents Q. In certain embodiments, in any one of Formulae (XLII) to (XLV), R^{4a} is —C(O)NR^{1b}R^{1c}; where R^{1b} and R^{1c} are each as defined herein. In certain embodiments, in any one of Formulae (XLII) to (XLV), R^{4a} is hydroxylmethyl, 1-hydroxylethyl, 1-hydroxy-1-methylethyl, methoxymethyl, acetoxymethyl, t-butylcarbonyloxy-methyl, valyloxymethyl, carbamoyloxymethyl, N-methylcarbamoyloxymethyl, methylsulfonyl-methyl, sulfamoylmethyl, N-methylsulfamoylmethyl, carbamoyl, methylcarbamoyl, or dimethylcarbamoyl. In certain embodiments, in any one of Formulae (XLII) to (XLV), R^{4a} is hydroxylmethyl, 1-hydroxylethyl, 1-hydroxy-1-methylethyl, methoxymethyl, acetoxymethyl, t-butylcarbonyloxymethyl, valyloxymethyl, carbamoyloxymethyl. N-methylcarbamoyloxymethyl, methylsulfonylmethyl, sulfamoylmethyl, or N-methylsulfamoylmethyl. In certain embodiments, in any one of Formulae (XLII) to (XLV). R^{4a} is hydroxylmethyl. In certain embodiments, in any one of Formulae (XLII) to (XLV), R^{4a} is valyloxymethyl. In certain embodiments, in any one of Formulae (XLII) to (XLV), R^{4a} is carbamoyloxymethyl.

The groups, ring R¹, R³, R^{2a}, R^{3a}, R^{4a}, R^{4b}, R⁵, R^{A}, A, E, L¹, L², U, V, X, Y, Z, m, n, and p, in formulae described herein, including Formulae (I) to (XLV), are further defined in the embodiments described herein. All combinations of the embodiments provided herein for such groups are within the scope of this disclosure.

In certain embodiments, R¹ is hydrogen. In certain embodiments, R¹ is deuterium. In certain embodiments, R¹ is C₁₋₆ alkyl, optionally substituted with one or more substituents Q. In certain embodiments, R¹ is methyl or dimethylaminomethyl. In certain embodiments, R¹ is C₂₋₆ alkenyl, optionally substituted with one or more substituents Q. In certain embodiments, R¹ is C₂₋₆ alkynyl, optionally substituted with one or more substituents Q. In certain embodiments, R¹ is C₃₋₁₀ cycloalkyl, optionally substituted with one or more substituents Q. In certain embodiments, R¹ is C₆₋₁₄ aryl, optionally substituted with one or more substituents Q. In certain embodiments, R¹ is C₇₋₁₅ aralkyl, optionally substituted with one or more substituents Q. In certain embodiments, R¹ is heteroaryl, optionally substituted with one or more substituents Q. In certain embodiments, R¹ is heterocyclyl, optionally substituted with one or more substituents Q. In certain embodiments, R¹ is hydrogen, deuterium, methyl, or dimethylaminomethyl.

In certain embodiments, R³ is hydrogen. In certain embodiments, R³ is deuterium. In certain embodiments, R³ is cyano. In certain embodiments, R³ is halo. In certain embodiments, R³ is fluoro, chloro, or bromo. In certain embodiments, R³ is nitro. In certain embodiments, R³ is hydrogen, deuterium, cyano, chloro, bromo, or nitro.

In certain embodiments, R³ is C₁₋₆ alkyl, optionally substituted with one or more substituents Q. In certain embodiments, R³ is methyl or ethyl, each optionally substituted with one or more substituents Q. In certain embodiments, R³ is methyl or ethyl, each optionally substituted with —C(O)OR^{a}, —C(O)NR^{b}R^{c}, or —R^{a}, wherein R^{a}, R^{b}, and R^{c} are each as defined herein. In certain embodiments, R³ is methyl or ethyl, each optionally substituted with methoxycarbonyl, carbamoyl, or hydroxyl. In certain embodiments, R³ is methyl, methoxycarbonylmethyl, carbamoylmethyl, hydroxymethyl, 2-methoxycarbonylethyl, or 2-hydroxylethyl. In certain embodiments, R³ is C₂₋₆ alkenyl, optionally substituted with one or more substituents Q. In certain embodiments, R³ is C₂₋₆ alkynyl, optionally substituted with one or more substituents Q. In certain embodiments, R³ is C₃₋₁₀ cycloalkyl, optionally substituted with one or more substituents Q.

In certain embodiments, R³ is C₆₋₁₄ aryl, optionally substituted with one or more substituents Q. In certain embodiments, R³ is phenyl, optionally substituted with one or more substituents Q. In certain embodiments, R³ is bicyclic C₆₋₁₄ aryl, optionally substituted with one or more substituents Q. In certain embodiments. R³ is 2,3-dihydroindenyl or naphthyl, each optionally substituted with one or more substituents Q. In certain embodiments, R³ is phenyl or 2,3-dihydroindenyl, each of which is optionally substituted with one or two substituents, wherein each substituent is independently cyano, oxo. C₃₋₁₀ cycloalkyl, heterocyclyl, —C(O)NR^{b}R^{c}, —NR^{b}R^{c}, —S(O)₂R^{a}, or —S(O)₂NR^{b}R^{c}; wherein the heterocyclyl is further optionally substituted with one, two, or three substituents Q^{a}; and R^{a}, R^{b}, and R^{c} are each as defined herein. In certain embodiments, R³ is phenyl or 2,3-dihydroinden-4-yl, each of which is optionally substituted with one or two substituents, wherein each substituent is independently cyano, fluoro, oxo, methyl, cyclopropyl, 1-cyanocyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, carbamoyl, methylcarbamoyl, (3-hydroxycyclobutyl)amino, oxetan-3-ylamino, methylsulfonyl, methylsulfamoyl, or dimethylsulfamoyl. In certain embodiments, R³ is phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(azetidin-1-yl)phenyl, 3-(pyrrolidin-1-yl)phenyl, 3-(3-hydroxypyrrolidin-1-yl)phenyl, 3-(2-oxopyrrolidin-1-yl)phenyl, 3-(2-oxoimidazolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(3-hydroxyazetidin-1-yl)phenyl, 3-carbamoyl-phenyl, 2-methylcarbamoylphenyl, 3-methylcarbamoylphenyl, 2-methylsulfamoylphenyl, 2-dimethylsulfamoylphenyl, 2-methylsulfonylphenyl, or 3-oxo-2,3-dihydro-1H-inden-4-yl. In certain embodiments, R³ is C₇₋₁₅ aralkyl, optionally substituted with one or more substituents Q.

In certain embodiments, R³ is heteroaryl, optionally substituted with one or more substituents Q. In certain embodiments, R³ is monocyclic heteroaryl, optionally substituted with one or more substituents Q. In certain embodiments, R³ is 5- or 6-membered heteroaryl, each optionally substituted with one or more substituents Q. In certain embodiments, R³ is pyrazolyl, thiazolyl, or pyridinyl, each of which is optionally substituted with one or two substituents Q. In certain embodiments, R³ is pyrazolyl, thiazolyl, or pyridinyl, each of which is optionally substituted with one or two C₁₋₆ alkyl. In certain embodiments, R³ is pyrazol-3-yl, pyrazol-4-yl, 1 thiazol-4-yl, thiazol-5-yl, or pyridin-3-yl, each of which is optionally substituted with methyl. In certain embodiments, R³ is pyrazol-3-yl, pyrazol-4-yl, 1-methylpyrazol-3-yl, 1-methyl-pyrazol-4-yl, thiazol-4-yl, thiazol-5-yl, or pyridin-3-yl.

In certain embodiments, R³ is bicyclic heteroaryl, optionally substituted with one or more substituents Q. In certain embodiments, R³ is 5,6- or 6,6-fused heteroaryl, each optionally substituted with one or more substituents Q. In certain embodiments, R³ is benzo[b]thiophenyl, benzo[d][1,2,3]thiadiazolyl, benzo[d]thiazolyl, imidazo[1,2-a]pyridinyl, imidazo[1,5-a]pyridinyl, indolyl, indazolyl, thiazolo[4,5-c]pyridinyl, [1,2,3]triazolo[1,5-a]pyridinyl, [1,2,4]triazolo[1,5-a]pyridinyl, [1,2,4]triazolo[4,3-a]pyridinyl, isoquinolinyl, quinolinyl, quinazolinyl, or quinoxalinyl, each of which is optionally substituted with one or two substituents, wherein each substituent is independently halo, oxo, C₁₋₆ alkyl, —C(O)OR^{a}, —C(O)NR^{b}R^{c}, or —OR^{a}; wherein the alkyl is optionally further substituted with one, two, or three substituents Q^{a}; and R^{a}, R^{b}, and R^{c} are each as defined herein. In certain embodiments, R³ is benzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, indazol-4-yl, thiazolo[4,5-c]pyridin-7-yl, [1,2,3]triazolo[1,5-a]pyridin-4-yl, [1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, isoquinolin-8-yl, quinolin-5-yl, quinazolin-5-yl, or quinoxalin-5-yl, each of which is optionally substituted with one or two substituents, wherein each substituent is independently cyano, fluoro, oxo, methyl, methoxycarbonyl, carbamoyl, methylcarbamoyl, or hydroxyl. In certain embodiments, R³ is 1,1-dioxidobenzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, 6-fluorobenzo[d]thiazol-5-yl, 6-cyanobenzo[d]thiazol-7-yl, 6-fluorobenzo[d]-thiazol-7-yl, 5-methoxycarbonylbenzo[d]thiazol-7-yl, 6-methoxycarbonylbenzo[d]thiazol-7-yl, 5-carbamoylbenzo[d]thiazol-7-yl, 6-carbamoyl-benzo[d]thiazol-7-yl, 5-methylcarbamoylbenzo[d]thiazol-7-yl, 6-methylcarbamoylbenzo[d]-thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethylindazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, [1,2,4]triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxyquinazolin-5-yl, or quinoxalin-5-yl. In certain embodiments, R³ is tricyclic heteroaryl, optionally substituted with one or more substituents Q. In certain embodiments, R³ is 8-oxo-7,8-dihydro-6H-thiazolo[5,4-e]isoindol-5-yl.

In certain embodiments, R³ is heterocyclyl, optionally substituted with one or more substituents Q. In certain embodiments, R³ is monocyclic heterocyclyl, optionally substituted with one or more substituents Q. In certain embodiments, R³ is 3-, 4-, 5-, 6-, or 7-membered heterocyclyl, each optionally substituted with one or more substituents Q. In certain embodiments, R³ is 5- or 6-membered heterocyclyl, each optionally substituted with one or more substituents Q. In certain embodiments, R³ is bicyclic heterocyclyl, optionally substituted with one or more substituents Q. In certain embodiments, R³ is 5,6- or 6,6-fused heterocyclyl, each optionally substituted with one or more substituents Q. In certain embodiments, R³ is 2,3-dihydrobenzo[b]thiophenyl, indolinyl, isoindolinyl, 2,3-dihydroindazolyl, dihydrobenzo[b]thiophenyl, or 3,4-dihydroquinazolinyl, each of which is optionally substituted with one, two, or three substituents, wherein each substituent is independently cyano, oxo, C_{J}0.6 alkyl, or —OR^{a}; wherein the alkyl is optionally further substituted with one, two, or three substituents Q^{a}; and R^{a} is as defined herein. In certain embodiments, R³ is 2,3-dihydrobenzo[b]thiophen-7-yl, isoindolin-4-yl, indolin-4-yl, 2,3-dihydroindazol-4-yl, dihydrobenzo[b]thiophen-7-yl, or 3,4-dihydroquinazolin-5-yl, each of which is optionally substituted with one, two, or three substituents, wherein each substituent is independently cyano, oxo, methyl, or hydroxyl. In certain embodiments, R³ is 3-hydroxy-1,1-dioxido-2,3-dihydrobenzo[b]thiophen-7-yl, 1,1-dioxido-3-oxo-2,3-dihydrobenzo[b]thiophen-7-yl, 1-oxoisoindolin-4-yl, 3-oxoisoindolin-4-yl, 2,3-dioxoindolin-4-yl, 2-methyl-1-oxoisoindolin-4-yl, 2-oxoindolin-4-yl, 1-methyl-2-oxoindolin-4-yl, 2,3-dihydro-1H-indazol-4-yl, 2,3-dihydrobenzo[b]-thiophen-7-yl, or 3-methyl-4-oxo-3,4-dihydroquinazolin-5-yl.

In certain embodiments, R³ is —C(O)R^{1a}, wherein R^{1a} is as defined herein. In certain embodiments, R³ is —C(O)OR^{1a}, wherein R^{1a} is as defined herein. In certain embodiments, R³ is —C(O)OC₁₋₆ alkyl, wherein the alkyl is optionally substituted with one or more substituents Q. In certain embodiments, R³ is methoxycarbonyl (—C(O)OCH₃). In certain embodiments, R³ is —C(O)NR^{1b}R^{1c}, wherein R^{1b} and R^{1c} are each as defined herein. In certain embodiments, R³ is —C(NR^{1a})NR^{1b}R^{1c}, wherein R^{1a}, R^{1b}, and R^{1c} are each as defined herein. In certain embodiments, R³ is —OR^{1a}, wherein R^{1a} is as defined herein. In certain embodiments, R³ is hydroxyl or —OC₁₋₆ alkyl, wherein the alkyl is optionally substituted with one or more substituents Q. In certain embodiments, R³ is hydroxyl or methoxy (—OCH₃). In certain embodiments, R³ is —OC(O)R^{1a}, wherein R^{1a} is as defined herein. In certain embodiments, R³ is —OC(O)OR^{1a}, wherein R^{1a} is as defined herein. In certain embodiments, R³ is —OC(O)NR^{1b}R^{1c}, wherein R^{1b} and R^{1c} are each as defined herein. In certain embodiments, R³ is —OC(NR^{1a})NR^{1b}R^{1c}, wherein R^{1a}, R^{1b}, and R^{1c} are each as defined herein. In certain embodiments, R³ is —OS(O)R^{1a}, wherein R^{1a} is as defined herein. In certain embodiments, R³ is —OS(O)₂R^{1a}, wherein R^{1a} is as defined herein. In certain embodiments, R³ is —OS(O)NR^{1b}R^{1c}, wherein R^{1b} and R^{1c} are each as defined herein. In certain embodiments, R³ is —OS(O)₂NR^{1b}R^{1c}, wherein R^{1b} and R^{1c} are each as defined herein. In certain embodiments, R³ is —NR^{1b}R^{1c}, wherein R^{1b} and R^{1c} are each as defined herein. In certain embodiments, R³ is amino (—NH₂). In certain embodiments, R³ is —NR^{1a}C(O)R^{1d}, wherein R^{1c} and R^{1d} are each as defined herein. In certain embodiments, R³ is —NHC(O)C₁₋₆ alkyl, wherein the alkyl is optionally substituted with one or more substituents Q. In certain embodiments, R³ is acetamido (—NHC(O)CH₃). In certain embodiments, R³ is —NR^{1a}C(O)OR^{1d}, wherein R^{1c} and R^{1d} are each as defined herein. In certain embodiments, R³ is —NR^{1a}C(O)NR^{1b}R^{1c}, wherein R^{1a}, R^{1b}, and R^{1c} are each as defined herein. In certain embodiments, R³ is —NR^{1a}C(NR^{1d})NR^{1b}R^{1c}, wherein R^{1a}, R^{1b}, R^{1c}, and R^{1d} are each as defined herein. In certain embodiments, R³ is —NR^{1a}S(O)R^{1d}, wherein R^{1c} and R^{1d} are each as defined herein. In certain embodiments, R³ is —NR^{1a}S(O)₂R^{1d}, wherein R^{1a} and R^{1d} are each as defined herein. In certain embodiments, R³ is —NHS(O)₂C₁₋₆ alkyl, wherein the alkyl is optionally substituted with one or more substituents Q. In certain embodiments, R³ is methylsulfonamido (—NHSO₂CH₃). In certain embodiments, R³ is —NR^{1a}S(O)NR^{1b}R^{1c}, wherein R^{1a}, R^{1b}, and R^{1c} are each as defined herein. In certain embodiments, R³ is —NR^{1a}S(O)₂NR^{1b}R^{1c}, wherein R^{1a}, R^{1b}, and R^{1c} are each as defined herein. In certain embodiments, R³ is —SR^{1a}, wherein R^{1a} is as defined herein. In certain embodiments, R³ is —S(O)R^{1a}, wherein R^{1a} is as defined herein. In certain embodiments, R³ is —S(O)₂R^{1a}, wherein R^{1a} is as defined herein. In certain embodiments, R³ is —S(O)₂C₁₋₆ alkyl, wherein the alkyl is optionally substituted with one or more substituents Q. In certain embodiments, R³ is methylsulfonyl (—SO₂CH₃). In certain embodiments, R³ is —S(O)NR^{1b}R^{1c}, wherein R^{1b} and R^{1c} are each as defined herein. In certain embodiments, R³ is —S(O)₂NR^{1b}R^{1c}, wherein R^{1b} and R^{1c} are each as defined herein. In certain embodiments, R³ is —S(O)₂NHC₁₋₆ alkyl, wherein the alkyl is optionally substituted with one or more substituents Q. In certain embodiments, R³ is methylsulfamoyl (—SO₂NHCH₃). In certain embodiments, R³ is methoxycarbonyl, hydroxyl, methoxy, amino, acetamido, methylsulfonamido, methylsulfonyl, or methylsulfamoyl.

In certain embodiments, (i) hydrogen, deuterium, cyano, chloro, bromo, or nitro; (ii) methyl, methoxycarbonylmethyl, carbamoylmethyl, hydroxymethyl, 2-methoxycarbonylethyl, 2-hydroxylethyl, phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(pyrrolidin-1-yl)phenyl, 3-(3-hydroxypyrrolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(azetidin-1-yl)phenyl, 3-(3-hydroxyazetidin-1-yl)phenyl, 3-(2-oxopyrrolidin-1-yl)phenyl, 3-(2-oxoimidazolidin-1-yl)phenyl, 3-carbamoylphenyl, 2-methylcarbamoylphenyl, 3-methylcarbamoylphenyl, 2-methylsulfamoylphenyl, 2-dimethylsulfamoylphenyl, 2-methyl-sulfonylphenyl, 3-oxo-2,3-dihydro-1H-inden-4-yl, pyrazol-3-yl, pyrazol-4-yl, 1-methylpyrazol-3-yl, 1-methylpyrazol-4-yl, thiazol-4-yl, thiazol-5-yl, pyridin-3-yl, 1,1-dioxidobenzo[b]-thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, 6-fluorobenzo[d]thiazol-5-yl, 6-cyanobenzo[d]thiazol-7-yl, 6-fluorobenzo[d]thiazol-7-yl, 5-methoxycarbonylbenzo[d]-thiazol-7-yl, 6-methoxycarbonylbenzo[d]thiazol-7-yl, 5-carbamoylbenzo[d]thiazol-7-yl, 6-carbamoylbenzo[d]thiazol-7-yl, 5-methylcarbamoylbenzo[d]thiazol-7-yl, 6-methylcarbamoyl-benzo[d]thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethyl-indazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, [1,2,4]triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxy-quinazolin-5-yl, quinoxalin-5-yl, 8-oxo-7,8-dihydro-6H-thiazolo[5,4-e]isoindol-5-yl, 3-hydroxy-1,1-dioxido-2,3-dihydrobenzo[b]thiophen-7-yl, 1,1-dioxido-3-oxo-2,3-dihydrobenzo[b]-thiophen-7-yl, 1-oxoisoindolin-4-yl, 3-oxoisoindolin-4-yl, 2-methyl-1-oxoisoindolin-4-yl, 2,3-dioxoindolin-4-yl, 2-oxoindolin-4-yl, 1-methyl-2-oxoindolin-4-yl, 1-methyl-3-oxo-2,3-dihydro-1H-indazol-4-yl, 2,2-difluoro-1,1-dioxido-3-oxo-2,3-dihydrobenzo[b]-thiophen-7-yl, or 3-methyl-4-oxo-3,4-dihydroquinazolin-5-yl; or (iii) methoxycarbonyl, hydroxyl, methoxy, amino, acetamido, methylsulfonamido, methylsulfonyl, or methylsulfamoyl.

In certain embodiments, R³ is

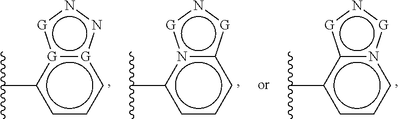

each of which is optionally substituted with one, two, or three substituents Q; wherein each G is independently —CR^{a}═, —N═, or —NR^{b}—; and wherein R^{a} and R^{b} are each as defined herein.

In certain embodiments, each R⁵ is independently deuterium. In certain embodiments, each R⁵ is independently cyano. In certain embodiments, each R⁵ is independently halo. In certain embodiments, each R⁵ is independently fluoro or chloro. In certain embodiments, each R⁵ is independently nitro. In certain embodiments, each R⁵ is independently C₁₋₆ alkyl, optionally substituted with one or more substituents Q. In certain embodiments, each R⁵ is independently hydroxymethyl. In certain embodiments, each R⁵ is independently C₂₋₆ alkenyl, optionally substituted with one or more substituents Q. In certain embodiments, each R⁵ is independently C₂₋₆ alkynyl, optionally substituted with one or more substituents Q. In certain embodiments, each R⁵ is independently C₃₋₁₀ cycloalkyl, optionally substituted with one or more substituents Q. In certain embodiments, each R⁵ is independently C₆₋₁₄ aryl, optionally substituted with one or more substituents Q. In certain embodiments, each R⁵ is independently C₇₋₁₄ aralkyl, optionally substituted with one or more substituents Q. In certain embodiments, each R⁵ is independently heteroaryl, optionally substituted with one or more substituents Q. In certain embodiments, each R⁵ is independently heterocyclyl, optionally substituted with one or more substituents Q. In certain embodiments, each R⁵ is independently hydrogen, methyl, or phenyl.

In certain embodiments, each R⁵ is independently —C(O)R^{1a}, wherein R^{1a} is as defined herein. In certain embodiments, each R⁵ is independently —C(O)OR^{1a}, wherein R^{1a} is as defined herein. In certain embodiments, each R⁵ is independently —C(O)NR^{1b}R^{1c}, wherein R^{1b} and R^{1c} are each as defined herein. In certain embodiments, each R⁵ is independently —C(NR^{1a})NR^{1b}R^{1c}, wherein R^{1a}, R^{1b}, and R^{1c} are each as defined herein. In certain embodiments, each R⁵ is independently —OR^{1a}, wherein R^{1a} is as defined herein. In certain embodiments, each R⁵ is independently —OC(O)R^{1a}, wherein R^{1a} is as defined herein. In certain embodiments, each R⁵ is independently —OC(O)OR^{1a}, wherein R^{1a} is as defined herein. In certain embodiments, each R⁵ is independently —OC(O)NR^{1b}R^{1c}, wherein R^{1b} and R^{c} are each as defined herein. In certain embodiments, each R⁵ is independently —OC(NR^{1a})NR^{1b}R^{1c}, wherein R^{1a}, R^{1b}, and R^{1c} are each as defined herein. In certain embodiments, each R⁵ is independently —OS(O)R^{1a}, wherein R^{1a} is as defined herein. In certain embodiments, each R⁵ is independently —OS(O)₂R^{1a}, wherein R^{1a} is as defined herein. In certain embodiments, each R⁵ is independently —OS(O)NR^{1b}R^{1c}, wherein R^{1b} and R^{1c} are each as defined herein. In certain embodiments, each R⁵ is independently —OS(O)₂NR^{1b}R^{1c}, wherein R^{1b} and R^{1c} are each as defined herein. In certain embodiments, each R⁵ is independently —NR^{1b}R^{1c}, wherein R^{1b} and R^{1c} are each as defined herein. In certain embodiments, each R⁵ is independently —NR^{1a}C(O)R^{1d}, wherein R^{1a} and R^{1d} are each as defined herein. In certain embodiments, each R⁵ is independently —NR^{1a}C(O)OR^{1d}, wherein R^{1a} and R^{1d} are each as defined herein. In certain embodiments, each R⁵ is independently —NR^{1a}C(O)NR^{1b}R^{1c}, wherein R^{1a}, R^{1b}, and R^{1c} are each as defined herein. In certain embodiments, each R⁵ is independently —NR^{1a}C(NR^{1d})NR^{1b}R^{1c}, wherein R^{1a}, R^{1b}, R^{1c}, and R^{1d} are each as defined herein. In certain embodiments, each R⁵ is independently —NR^{1a}S(O)R^{1d}, wherein R^{1a} and R^{1d} are each as defined herein. In certain embodiments, each R⁵ is independently —NR¹³S(O)₂R^{1d}, wherein R^{1a} and R^{1d} are each as defined herein. In certain embodiments, each R⁵ is independently —NR^{1a}S(O)NR^{1b}R^{1c}, wherein R^{1a}, R^{1b}, and R^{1c} are each as defined herein. In certain embodiments, each R⁵ is independently —NR^{1a}S(O)₂NR^{1b}R^{1c}, wherein R^{1a}, R^{1b}, and R^{1c} are each as defined herein. In certain embodiments, each R⁵ is independently —SR^{1a}, wherein R^{1a} is as defined herein. In certain embodiments, each R⁵ is independently —S(O)R^{1a}, wherein R^{1a} is as defined herein. In certain embodiments, each R⁵ is independently —S(O)₂R^{1a}, wherein R^{1a} is as defined herein. In certain embodiments, each R⁵ is independently —S(O)NR^{1b}R^{1c}, wherein R^{1b} and R^{1c} are each as defined herein. In certain embodiments, each R⁵ is independently —S(O)₂NR^{1b}R^{1c}, wherein R^{1b} and R^{1c} are each as defined herein.

In certain embodiments, each R^{2a} is independently hydrogen. In certain embodiments, each R^{2a} is independently deuterium. In certain embodiments, each R^{2a} is independently cyano. In certain embodiments, each R^{2a} is independently halo. In certain embodiments, each R^{2a} is independently fluoro or chloro. In certain embodiments, each R^{2a} is independently nitro. In certain embodiments, each R^{2a} is independently C₁₋₆ alkyl, optionally substituted with one or more substituents Q. In certain embodiments, each R^{2a} is independently methyl. In certain embodiments, each R^{2a} is independently C₂₋₆ alkenyl, optionally substituted with one or more substituents Q. In certain embodiments, each R^{2a} is independently C₂₋₆ alkynyl, optionally substituted with one or more substituents Q. In certain embodiments, each R^{2a} is independently C₃₋₁₀ cycloalkyl, optionally substituted with one or more substituents Q. In certain embodiments, each R^{2a} is independently C₆₋₄ aryl, optionally substituted with one or more substituents Q. In certain embodiments, each R^{2a} is independently phenyl. In certain embodiments, each R^{2a} is independently C₇₋₁₅ aralkyl, optionally substituted with one or more substituents Q. In certain embodiments, each R^{2a} is independently heteroaryl, optionally substituted with one or more substituents Q. In certain embodiments, each R^{2a} is independently heterocyclyl, optionally substituted with one or more substituents Q. In certain embodiments, each R^{2a} is independently hydrogen, methyl, or phenyl.

In certain embodiments, each R^{2a} is independently —C(O)R^{1a}, wherein R^{1a} is as defined herein. In certain embodiments, each R^{2a} is independently —C(O)OR^{1a}, wherein R^{1a} is as defined herein. In certain embodiments, each R^{2a} is independently —C(O)NR^{1b}R^{1c}, wherein R^{1b} and R^{1c} are each as defined herein. In certain embodiments, each R^{2a} is independently —C(NR^{1a})NR^{1b}R^{1c}, wherein R^{1a}, R^{1b}, and R^{1c} are each as defined herein. In certain embodiments, each R^{2a} is independently —OR^{1a}, wherein R^{1a} is as defined herein. In certain embodiments, each R^{1a} is independently —OC(O)R^{1a}, wherein R^{1a} is as defined herein. In certain embodiments, each R^{1a} is independently —OC(O)OR^{1a}, wherein R^{1a} is as defined herein. In certain embodiments, each R^{2a} is independently —OC(O)NR^{1b}R^{1c}, wherein R^{1b} and R^{1c} are each as defined herein. In certain embodiments, each R^{2a} is independently —OC(NR^{1a})NR^{1b}R^{1c}, wherein R^{1a}, R^{1b}, and R^{1c} are each as defined herein. In certain embodiments, each R^{2a} is independently —OS(O)R^{1a}, wherein R^{1a} is as defined herein. In certain embodiments, each R^{2a} is independently —OS(O)₂R^{1a}, wherein R^{1a} is as defined herein. In certain embodiments, each R^{2a} is independently —OS(O)NR^{1b}R^{1c}, wherein R^{1b} and R^{1c} are each as defined herein. In certain embodiments, each R^{2a} is independently —OS(O)₂NR^{1b}R^{1c}, wherein R^{1b} and R^{1c} are each as defined herein. In certain embodiments, each R^{2a} is independently —NR^{1b}R^{1c}, wherein R^{1b} and R^{1c} are each as defined herein. In certain embodiments, each R^{2a} is independently —NR^{1a}C(O)R^{1d}, wherein R^{1a} and R^{1d} are each as defined herein. In certain embodiments, each R^{2a} is independently —NR^{1a}C(O)OR^{1d}, wherein R^{1a} and R^{1d} are each as defined herein. In certain embodiments, each R^{2a} is independently —NR^{1a}C(O)NR^{1b}R^{1c}, wherein R^{1a}, R^{1b}, and R^{1c} are each as defined herein. In certain embodiments, each R^{2a} is independently —NR^{1a}C(NR^{1d})NR^{1b}R^{1c}, wherein R^{1a}, R^{1b}, R^{1c}, and R^{1d} are each as defined herein. In certain embodiments, each R^{2a} is independently —NR^{1a}S(O)R^{1d}, wherein R^{1a} and R^{1d} are each as defined herein. In certain embodiments, each R^{2a} is independently —NR^{1a}S(O)₂R^{1d}, wherein R^{1a} and R^{1d} are each as defined herein. In certain embodiments, each R^{2a} is independently —NR^{1a}S(O)NR^{1b}R^{1c}, wherein R^{1a}, R^{1b}, and R^{1c} are each as defined herein. In certain embodiments, each R^{2a} is independently —NR^{1a}S(O)₂NR^{1b}R^{1c}, wherein R^{1a}, R^{1b}, and R^{1c} are each as defined herein. In certain embodiments, each R^{2a} is independently —SR^{1a}, wherein R^{1a} is as defined herein. In certain embodiments, each R^{2a} is independently —S(O)R^{1a}, wherein R^{1a} is as defined herein. In certain embodiments, each R^{2a} is independently —S(O)₂R^{1a}, wherein R^{1a} is as defined herein. In certain embodiments, each R^{2a} is independently —S(O)NR^{1b}R^{1c}, wherein R^{1b} and R^{1c} are each as defined herein. In certain embodiments, each R^{2a} is independently —S(O)₂NR^{1b}R^{1c}, wherein R^{1b} and R^{1c} are each as defined herein.

In certain embodiments, each R^{3a} is independently deuterium. In certain embodiments, each R^{3a} is independently cyano. In certain embodiments, each R^{3a} is independently halo. In certain embodiments, each R^{3a} is independently fluoro or chloro. In certain embodiments, each R^{3a} is independently nitro. In certain embodiments, each R^{3a} is independently C₁₋₆ alkyl, optionally substituted with one or more substituents Q. In certain embodiments, each R^{3a} is independently C₂₋₆ alkenyl, optionally substituted with one or more substituents Q. In certain embodiments, each R^{3a} is independently C₂₋₆ alkynyl, optionally substituted with one or more substituents Q. In certain embodiments, each R^{3a} is independently C₃₋₁₀ cycloalkyl, optionally substituted with one or more substituents Q. In certain embodiments, each R^{3a} is independently C₆₋₁₄ aryl, optionally substituted with one or more substituents Q. In certain embodiments, each R^{3a} is independently C₇₋₁₅ aralkyl, optionally substituted with one or more substituents Q. In certain embodiments, each R^{3a} is independently heteroaryl, optionally substituted with one or more substituents Q. In certain embodiments, each R^{3a} is independently heterocyclyl, optionally substituted with one or more substituents Q. In certain embodiments, each R^{3a} is independently hydrogen, methyl, or phenyl.

In certain embodiments, each R^{3a} is independently —C(O)R^{1a}, wherein R^{1a} is as defined herein. In certain embodiments, each R^{3a} is independently —C(O)OR^{1a}, wherein R^{1a} is as defined herein. In certain embodiments, each R^{3a} is independently —C(O)NR^{1b}R^{1c}, wherein R^{1b} and R^{1c} are each as defined herein. In certain embodiments, each R³ is independently —C(NR^{1a})NR^{1b}R^{1c}, wherein R^{1a}, R^{1b}, and R^{1c} are each as defined herein. In certain embodiments, each R^{3a} is independently —OR^{1a}, wherein R^{1a} is as defined herein. In certain embodiments, each R^{3a} is independently —OC(O)R^{1a}, wherein R^{1a} is as defined herein. In certain embodiments, each R^{3a} is independently —OC(O)OR^{1a}, wherein R^{1a} is as defined herein. In certain embodiments, each R^{3a} is independently —OC(O)NR^{1b}R^{1c}, wherein R^{1b} and R^{1a} are each as defined herein. In certain embodiments, each R^{3a} is independently —OC(NR^{1a})NR^{1b}R^{1c}, wherein R^{1a}, R^{1b}, and R^{1c} are each as defined herein. In certain embodiments, each R^{3a} is independently —OS(O)R^{1a}, wherein R^{1a} is as defined herein. In certain embodiments, each R^{3a} is independently —OS(O)₂R^{1a}, wherein R^{1a} is as defined herein. In certain embodiments, each R^{3a} is independently —OS(O)NR^{1b}R^{1c}, wherein R^{1b} and R^{1c} are each as defined herein. In certain embodiments, each R^{3a} is independently —OS(O)₂NR^{1b}R^{1c}, wherein R^{1b} and R^{1c} are each as defined herein. In certain embodiments, each R^{3a} is independently —NR^{1b}R^{1c}, wherein R^{1b} and R^{1c} are each as defined herein. In certain embodiments, each R^{3a} is independently —NR^{1a}C(O)R^{1d}, wherein R^{1a} and R^{1d} are each as defined herein. In certain embodiments, each R^{3a} is independently —NR^{1a}C(O)OR^{1d}, wherein R^{1a} and R^{1d} are each as defined herein. In certain embodiments, each R^{3a} is independently —NR^{1a}C(O)NR^{1b}R^{1c}, wherein R^{1a}, R^{1b}, and R^{1c} are each as defined herein. In certain embodiments, each R^{3a} is independently —NR^{1a}C(NR^{1d})NR^{1b}R^{1c}, wherein R^{1a}, R^{1b}, R^{1c}, and R^{1d} are each as defined herein. In certain embodiments, each R^{3a} is independently —NR^{1a}S(O)R^{1d}, wherein R^{1a} and R^{1d} are each as defined herein. In certain embodiments, each R^{3a} is independently —NR^{1a}S(O)₂R^{1d}, wherein R^{1a} and R^{1d} are each as defined herein. In certain embodiments, each R^{3a} is independently —NR^{1a}S(O)NR^{1b}R^{1c}, wherein R^{1a}, R^{1b}, and R^{1c} are each as defined herein. In certain embodiments, each R^{3a} is independently —NR^{1a}S(O)₂NR^{1b}R^{1c}, wherein R^{1a}, R^{1b}, and R^{1c} are each as defined herein. In certain embodiments, each R^{3a} is independently —SR^{1a}, wherein R^{1a} is as defined herein. In certain embodiments, each R^{3a} is independently —S(O)R^{1a}, wherein R^{1a} is as defined herein. In certain embodiments, each R^{3a} is independently —S(O)₂R^{1a}, wherein R^{1a} is as defined herein. In certain embodiments, each R^{3a} is independently —S(O)NR^{1b}R^{1c}, wherein R^{1b} and R^{1c} are each as defined herein. In certain embodiments, each R^{3a} is independently —S(O)₂NR^{1b}R^{1c}, wherein R^{1b} and R^{1c} are each as defined herein.

In certain embodiments, each R^{4a} is independently hydrogen. In certain embodiments, each R^{4a} is independently deuterium. In certain embodiments, each R^{4a} is independently cyano. In certain embodiments, each R^{4a} is independently halo. In certain embodiments, each R^{4a} is independently fluoro or chloro. In certain embodiments, each R^{4a} is independently nitro. In certain embodiments, each R^{4a} is independently C₁₋₆ alkyl, optionally substituted with one or more substituents Q. In certain embodiments, each R^{4a} is independently methyl, optionally substituted with one or more substituents Q. R^{4a} is hydroxylmethyl, 1-hydroxylethyl, 1-hydroxy-1-methylethyl, methoxymethyl, acetoxymethyl, t-butylcarbonyloxy-methyl, valyloxymethyl, carbamoyloxymethyl, N-methylcarbamoyloxymethyl, methylsulfonyl-methyl, sulfamoylmethyl, or N-methylsulfamoylmethyl. In certain embodiments, each R^{4a} is independently C₂₋₆ alkenyl, optionally substituted with one or more substituents Q. In certain embodiments, each R^{4a} is independently C₂₋₆ alkynyl, optionally substituted with one or more substituents Q. In certain embodiments, each R⁴, is independently C₃₋₁₀ cycloalkyl, optionally substituted with one or more substituents Q. In certain embodiments, each R⁴ is independently C₆₋₁₄ aryl, optionally substituted with one or more substituents Q. In certain embodiments, each R^{4a} is independently C₇₋₁₅ aralkyl, optionally substituted with one or more substituents Q. In certain embodiments, each R^{4a} is independently heteroaryl, optionally substituted with one or more substituents Q. In certain embodiments, each R^{4a} is independently heterocyclyl, optionally substituted with one or more substituents Q. In certain embodiments, each R^{4a} is independently hydrogen, methyl, or phenyl.

In certain embodiments, each R^{4a} is independently —C(O)R^{1a}, wherein R^{1a} is as defined herein. In certain embodiments, each R^{4a} is independently —C(O)OR^{1a}, wherein R^{1a} is as defined herein. In certain embodiments, each R^{4a} is independently —C(O)NR^{1b}R^{1c}, wherein R^{1b} and R^{1c} are each as defined herein. In certain embodiments, each R^{4a} is independently carbamoyl, methylcarbamoyl, or dimethylcarbamoyl. In certain embodiments, each R^{4a} is independently —C(NR^{1a})NR^{1b}R^{1c}, wherein R^{1a}, R^{1b}, and R^{1c} are each as defined herein. In certain embodiments, each R^{4a} is independently —OR^{1a}, wherein R^{1a} is as defined herein. In certain embodiments, each R^{4a} is independently hydroxyl. In certain embodiments, each R^{4a} is independently —OC(O)R^{1a}, wherein R^{1a} is as defined herein. In certain embodiments, each R^{4a} is independently —OC(O)OR^{1a}, wherein R^{1a} is as defined herein. In certain embodiments, each R^{4a} is independently —OC(O)NR^{1b}R^{1c}, wherein R^{1b} and R^{1c} are each as defined herein. In certain embodiments, each R^{4a} is independently —OC(NR^{1a})NR^{1b}R^{1c}, wherein R^{1a}, R^{1b}, and R^{1c} are each as defined herein. In certain embodiments, each R^{4a} is independently —OS(O)R^{1a}, wherein R^{1a} is as defined herein. In certain embodiments, each R^{4a} is independently —OS(O)₂R^{1a}, wherein R^{1a} is as defined herein. In certain embodiments, each R^{4a} is independently —OS(O)NR^{1b}R^{1c}, wherein R^{1b} and R^{1c} are each as defined herein. In certain embodiments, each R^{4a} is independently —OS(O)₂NR^{1b}R^{1c}, wherein R^{1b} and R^{1c} are each as defined herein. In certain embodiments, each R^{4a} is independently —NR^{1b}R^{1c}, wherein R^{1b} and R^{1c} are each as defined herein. In certain embodiments, each R^{4a} is independently amino. In certain embodiments, each R^{4a} is independently —NR^{1a}C(O)R^{1d}, wherein R^{1c} and R^{1d} are each as defined herein. In certain embodiments, each R^{4a} is independently —NR^{1a}C(O)OR^{1d}, wherein R^{1a} and R^{1d} are each as defined herein. In certain embodiments, each R^{4a} is independently —NR^{1a}C(O)NR^{1b}R^{1c}, wherein R^{1a}, R^{1b}, and R^{1c} are each as defined herein. In certain embodiments, each R^{4a} is independently —NR^{1a}C(NR^{1d})NR^{1b}R^{1c}, wherein R^{1a}, R^{1b}, R^{1c}, and R^{1d} are each as defined herein. In certain embodiments, each R^{1a} is independently —NR^{1a}S(O)R^{1d}, wherein R^{1c} and R^{1d} are each as defined herein. In certain embodiments, each R^{1a} is independently —NR^{1a}S(O)₂R^{1d}, wherein R^{1a} and R^{1d} are each as defined herein. In certain embodiments, each R^{4a} is independently —NR^{1a}S(O)NR^{1b}R^{1c}, wherein R^{1a}, R^{1b}, and R^{1c} are each as defined herein. In certain embodiments, each R^{1a} is independently —NR^{1a}S(O)₂NR^{1b}R^{1c}, wherein R^{1a}, R^{1b}, and R^{1c} are each as defined herein. In certain embodiments, each R^{4a} is independently —SR^{1a}, wherein R^{1a} is as defined herein. In certain embodiments, each R^{4a} is independently —S(O)R^{1a}, wherein R^{1a} is as defined herein. In certain embodiments, each R^{4a} is independently —S(O)₂R^{1a}, wherein R^{1a} is as defined herein. In certain embodiments, each R^{4a} is independently —S(O)NR^{1b}R^{1c}, wherein R^{1b} and R^{1c} are each as defined herein. In certain embodiments, each R^{4a} is independently —S(O)₂NR^{1b}R^{1c}, wherein R^{1b} and R^{1c} are each as defined herein.

In certain embodiments, each R^{4b} is independently hydrogen. In certain embodiments, each R^{4b} is independently deuterium. In certain embodiments, each R^{4b} is independently cyano. In certain embodiments, each R^{4b} is independently halo. In certain embodiments, each R^{4b} is independently fluoro or chloro. In certain embodiments, each R^{4b} is independently nitro. In certain embodiments, each R^{4b} is independently C₁₋₆ alkyl, optionally substituted with one or more substituents Q. In certain embodiments, each R^{b} is independently methyl. In certain embodiments, each R^{4b} is independently C₂₋₆ alkenyl, optionally substituted with one or more substituents Q. In certain embodiments, each R^{4b} is independently C₂₋₆ alkynyl, optionally substituted with one or more substituents Q. In certain embodiments, each R^{4b} is independently C₃₋₁₀ cycloalkyl, optionally substituted with one or more substituents Q. In certain embodiments, each R^{4b} is independently C₆₋₁₄ aryl, optionally substituted with one or more substituents Q. In certain embodiments, each R^{4b} is independently C₇₋₁₅ aralkyl, optionally substituted with one or more substituents Q. In certain embodiments, each R^{4b} is independently heteroaryl, optionally substituted with one or more substituents Q. In certain embodiments, each R^{4b} is independently heterocyclyl, optionally substituted with one or more substituents Q. In certain embodiments, each R^{4b} is independently hydrogen, methyl, or phenyl.

In certain embodiments, each R^{4b} is independently —C(O)R^{1a}, wherein R^{1a} is as defined herein. In certain embodiments, each R^{4b} is independently —C(O)OR^{1a}, wherein R^{1a} is as defined herein. In certain embodiments, each R^{4b} is independently —C(O)NR^{1b}R^{1c}, wherein R^{1b} and R^{1c} are each as defined herein. In certain embodiments, each R^{4b} is independently —C(NR^{1a})NR^{1b}R^{1c}, wherein R^{1a}, R^{1b}, and R^{1c} are each as defined herein. In certain embodiments, each R^{4b} is independently —OR^{1a}, wherein R^{1a} is as defined herein. In certain embodiments, each R^{4b} is independently hydroxyl. In certain embodiments, each R^{4b} is independently —OC(O)R^{1a}, wherein R^{1a} is as defined herein. In certain embodiments, each R^{4b} is independently —OC(O)OR^{1a}, wherein R^{1a} is as defined herein. In certain embodiments, each R^{4b} is independently —OC(O)NR^{1b}R^{1c}, wherein R^{1b} and R^{1c} are each as defined herein. In certain embodiments, each R^{4b} is independently —OC(NR¹²)NR^{1b}R^{1c}, wherein R^{1a}, R^{1b}, and R^{1c} are each as defined herein. In certain embodiments, each R^{4b} is independently —OS(O)R^{1a}, wherein R^{1a} is as defined herein. In certain embodiments, each R^{4b} is independently —OS(O)₂R^{1a}, wherein R^{1a} is as defined herein. In certain embodiments, each R^{4b} is independently —OS(O)NR^{1b}R^{1c}, wherein R^{1b} and R^{1c} are each as defined herein. In certain embodiments, each R^{4b} is independently —OS(O)₂NR^{1b}R^{1c}, wherein R^{1b} and R^{c} are each as defined herein. In certain embodiments, each R^{4b} is independently —NR^{1b}R^{1c}, wherein R^{1b} and R^{1c} are each as defined herein. In certain embodiments, each R^{1a} is independently amino. In certain embodiments, each R^{4b} is independently —NR^{1a}C(O)R^{1d}, wherein R^{1a} and R^{1d} are each as defined herein. In certain embodiments, each R^{1a} is independently —NR^{1a}C(O)OR^{1d}, wherein R^{1a} and R^{1d} are each as defined herein. In certain embodiments, each R^{4b} is independently —NR^{1a}C(O)NR^{1b}R^{1c}, wherein R^{1a}, R^{1b}, and R^{1c} are each as defined herein. In certain embodiments, each R^{4b} is independently —NR^{1a}C(NR^{1d})NR^{1b}R^{1c}, wherein R^{1a}, R^{1b}, R^{1c}, and R^{1d} are each as defined herein. In certain embodiments, each R^{4b} is independently —NR^{1a}S(O)R^{1d}, wherein R^{1a} and R^{1d} are each as defined herein. In certain embodiments, each R^{4b} is independently —NR^{1a}S(O)₂R^{1d}, wherein R^{1a} and R^{1d} are each as defined herein. In certain embodiments, each R^{4b} is independently —NR^{1a}S(O)NR^{1b}R^{1c}, wherein R^{1a}, R^{1b}, and R^{1c} are each as defined herein. In certain embodiments, each R^{4b} is independently —NR^{1a}S(O)₂NR^{1b}R^{1c}, wherein R^{1a}, R^{1b}, and R^{c} are each as defined herein. In certain embodiments, each R^{4b} is independently —SR^{1a}, wherein R^{1a} is as defined herein. In certain embodiments, each R^{4b} is independently —S(O)R^{1a}, wherein R^{1a} is as defined herein. In certain embodiments, each R^{4b} is independently —S(O)₂R^{1a}, wherein R^{1a} is as defined herein. In certain embodiments, each R^{4b} is independently —S(O)NR^{1b}R^{1c}, wherein R^{1b} and R^{1c} are each as defined herein. In certain embodiments, each R^{4b} is independently —S(O)₂NR^{1b}R^{1c}, wherein R^{1b} and R^{1c} are each as defined herein.

In certain embodiments, R⁴ and R^{4b} together with the carbon atom to which they are attached form monocyclic C₃₋₁₀ cycloalkylene, optionally substituted with one or more substituents Q. In certain embodiments, R⁴ and R^{4b} together with the carbon atom to which they are attached form monocyclic C₃₋₁₀ cycloalkylene, optionally substituted with one or more substituents Q. In certain embodiments, R^{4a} and R^{4b} together with the carbon atom to which they are attached form cyclopropanediyl.

In certain embodiments, each R^{A} is independently hydrogen. In certain embodiments, each R^{A} is independently C₁₋₆ alkyl, optionally substituted with one ore substituents Q. In certain embodiments, each R^{A} is independently methyl.

In certain embodiments, A is —C(O)—. In certain embodiments, A is —C(O)NR^{1a}_, wherein R^{1a} is as defined herein. In certain embodiments, A is —C(O)NH—. In certain embodiments, A is —C(O)N(C₁₋₆ alkyl)-, wherein the alkyl is optionally substituted with one or more substituents Q. In certain embodiments, A is —C(O)N(C₁₋₆ alkyl)-, wherein the alkyl is optionally substituted with —C(O)OR^{1a} or —OR^{1a}; and wherein each R^{1a} is as defined herein. In certain embodiments, A is —C(O)NR^{1a}—, wherein R^{1a} is methyl, ethoxycarbonylmethyl, or 2-hydroxyethyl. In certain embodiments, A is —OC(O)NR^{1a}—, wherein R^{1a} is as defined herein. In certain embodiments, A is —OC(O)NH—. In certain embodiments, A is —NR^{1a}C(O)NR^{1d}—, wherein R^{1a} and R^{1d} are each as defined herein. In certain embodiments, A is —NHC(O)NH—. In certain embodiments, A is —NHC(O)N(CH₃)—. In certain embodiments, A is —N(CH₃)C(O)N(CH₃)—. In certain embodiments, A is —S(O)—. In certain embodiments, A is —S(O)₂—. In certain embodiments, A is —S(O)NR^{1a}—, wherein R^{1a} is as defined herein. In certain embodiments, A is —S(O)NH—. In certain embodiments, A is —S(O)₂NR^{1a}—, wherein R^{1a} is as defined herein. In certain embodiments, A is —S(O)₂NH—. In certain embodiments, A is —C(O)—, —C(O)NH—, —C(O)N(CH₃)—, or —NHC(O)NH—.

In certain embodiments, E is —C(H)═. In certain embodiments, E is —N═.

In certain embodiments, L¹ is a bond. In certain embodiments, L¹ is C₁₋₆ alkylene, optionally substituted with one or more substituents Q. In certain embodiments, L¹ is methanediyl or ethanediyl, each of which is optionally substituted with —OR^{a} or —NR^{b}R^{c}, wherein R^{a}, R^{b}, and R^{c} are each as defined herein. In certain embodiments, L¹ is methanediyl, ethane-1,1-diyl, or ethane-1,2-diyl, each of which is optionally substituted with hydroxyl or amino. In certain embodiments, L¹ is methanediyl, ethane-1,1-diyl, 2-hydroxyethane-1,1-diyl, ethane-1,2-diyl, 1-hydroxyethane-1,2-diyl, or 1-aminoethane-1,2-diyl. In certain embodiments, L¹ is C₂₋₆ alkenylene, optionally substituted with one or more substituents Q. In certain embodiments, L¹ is ethenediyl, optionally substituted with one or more substituents Q. In certain embodiments, L¹ is ethene-1,2-diyl, optionally substituted with one or more substituents Q. In certain embodiments, L¹ is C₂₋₆ alkynylene, optionally substituted with one or more substituents Q.

In certain embodiments, L¹ is C₃₋₁₀ cycloalkylene, optionally substituted with one or more substituents Q. In certain embodiments, L¹ is monocyclic C₃₋₁₀ cycloalkylene, optionally substituted with one or more substituents Q. In certain embodiments, L¹ is cyclopropanediyl, optionally substituted with one or more substituents Q. In certain embodiments, L¹ is cyclopropane-1,1-diyl, optionally substituted with one or more substituents Q. In certain embodiments, L¹ is C₆₋₁₄ arylene, optionally substituted with one or more substituents Q. In certain embodiments, L¹ is phendiyl, optionally substituted with one or more substituents Q. In certain embodiments, L¹ is bicyclic C₉₋₁₄ arylene, optionally substituted with one or more substituents Q. In certain embodiments, L¹ is heteroarylene, optionally substituted with one or more substituents Q. In certain embodiments, L¹ is monocyclic heteroarylene, optionally substituted with one or more substituents Q. In certain embodiments, L¹ is 5- or 6-membered heteroarylene, each optionally substituted with one or more substituents Q. In certain embodiments, L¹ is bicyclic heteroarylene, optionally substituted with one or more substituents Q. In certain embodiments, L¹ is 5,6- or 6,6-fused heteroarylene, each optionally substituted with one or more substituents Q.

In certain embodiments, L¹ is heterocyclylene, optionally substituted with one or more substituents Q. In certain embodiments, L¹ is monocyclic heterocyclylene, optionally substituted with one or more substituents Q. In certain embodiments, L¹ is 3-, 4-, 5-, 6-, or 7-membered heterocyclylene, each optionally substituted with one or more substituents Q. In certain embodiments, L¹ is 4-, 5-, or 6-membered heterocyclylene, each optionally substituted with one or more substituents Q. In certain embodiments, L¹ is bicyclic heterocyclylene, optionally substituted with one or more substituents Q. In certain embodiments, L¹ is 5,6- or 6,6-fused heterocyclylene, each optionally substituted with one or more substituents Q. In certain embodiments, L¹ is azetidindiyl, pyrrolidindiyl, piperidindiyl, or piperazindiyl, each of which is optionally substituted with halo, C₁₋₆ alkyl, or —OR^{a}; wherein the alkyl is optionally substituted with one or more substituents Q and R^{a} is as defined herein. In certain embodiments, L¹ is azetidin-1,3-diyl, pyrrolidin-1,2-diyl, piperidin-1,4-diyl, or piperazin-1,4-diyl, each of which is optionally substituted with fluoro, hydroxymethyl, or hydroxyl. In certain embodiments, L¹ is azetidin-1,3-diyl, pyrrolidin-1,2-diyl, piperidin-1,4-diyl, 4-fluropiperidin-1,4-diyl, 4-hydroxypiperidin-1,4-diyl, piperazin-1,4-diyl, or 2-hydroxymethylpiperazin-1,4-diyl. In certain embodiments, L¹ is a bond, methanediyl, ethane-1,1-diyl, ethane-1,2-diyl, 1-hydroxyethane-1,2-diyl, 1-aminoethane-1,2-diyl, ethene-1,2-diyl, cyclopropan-1,1-diyl, azetidin-1,3-diyl, pyrrolidin-1,2-diyl, piperidin-1,4-diyl, 4-fluropiperidin-1,4-diyl, 4-hydroxypiperidin-1,4-diyl, piperazin-1,4-diyl, or 2-hydroxymethylpiperazin-1,4-diyl.

In certain embodiments, L² is C₆₋₁₄ arylene, optionally substituted with one or more substituents Q. In certain embodiments, L² is phendiyl, optionally substituted with one or more substituents Q. In certain embodiments, L² is bicyclic C₆₋₁₄ arylene, optionally substituted with one or more substituents Q. In certain embodiments, L² is 2,3-dihydroindendiyl or naphthdiyl, each optionally substituted with one or more substituents Q. In certain embodiments, L² is phendiyl, 2,3-dihydroindendiyl, or naphthdiyl, each of which is optionally substituted with one or two substituents, wherein each substituent is independently cyano, halo, or —OR^{a}; and wherein R^{a} is as defined herein. In certain embodiments, L² is phen-1,2-diyl, phen-1,3-diyl, phen-1,4-diyl, 2,3-dihydroinden-1,4-diyl, 2,3-dihydroinden-2,5-diyl, naphth-1,5-diyl, or naphth-2,6-diyl, each of which is optionally substituted with one or two substituents, wherein each substituent is independently cyano, fluoro, chloro, hydroxyl, or methoxy. In certain embodiments, L² is phen-1,2-diyl, phen-1,3-diyl, phen-1,4-diyl, 4-methoxyphen-1,3-diyl, 2-cyanophen-1,4-diyl, 2-fluorophen-1,4-diyl, 2-chlorophen-1,4-diyl, 2-hydroxyphen-1,4-diyl, 2,3-dihydroinden-1,4-diyl, 2,3-dihydroinden-2,5-diyl, naphth-1,5-diyl, or naphth-2,6-diyl.

In certain embodiments, L² is heteroarylene, optionally substituted with one or more substituents Q. In certain embodiments, L² is monocyclic heteroarylene, optionally substituted with one or more substituents Q. In certain embodiments, L² is 5- or 6-membered heteroarylene, each optionally substituted with one or more substituents Q. In certain embodiments, L² is pyrazoldiyl or pyridindiyl, each optionally substituted with one or more substituents Q. In certain embodiments, L² is pyrazol-1,3-diyl, pyrazol-1,4-diyl, pyridin-2,3-diyl, or pyridin-2,5-diyl, each optionally substituted with one or more substituents Q. In certain embodiments, L² is bicyclic heteroarylene, optionally substituted with one or more substituents Q. In certain embodiments, L² is 5,6- or 6,6-fused heteroarylene, each optionally substituted with one or more substituents Q. In certain embodiments, L² is indoldiyl, indazoldiyl, benzothiazoldiyl, quinoldiyl, or quinoldiyl, each optionally substituted with one or more substituents Q. In certain embodiments, L² is indol-2,5-diyl, indazol-3,7-diyl, benzothiazol-2,6-diyl, quinol-2,6-diyl, or quinol-3,7-diyl, each optionally substituted with one or more substituents Q.

In certain embodiments, L² is heterocyclylene, optionally substituted with one or more substituents Q. In certain embodiments, L² is monocyclic heterocyclylene, optionally substituted with one or more substituents Q. In certain embodiments, L² is 3-, 4-, 5-, 6-, or 7-membered heterocyclylene, each optionally substituted with one or more substituents Q. In certain embodiments, L² is 5- or 6-membered heterocyclylene, each optionally substituted with one or more substituents Q. In certain embodiments, L² is bicyclic heterocyclylene, optionally substituted with one or more substituents Q. In certain embodiments. L¹ is 5,6- or 6,6-fused heterocyclylene, each optionally substituted with one or more substituents Q. In certain embodiments, L² is piperidindiyl, isoindolindiyl, 1,2,3,4-tetrahydroisoquinolindiyl, benzo[d][1,3]dioxoldiyl, or 2,3-dihydrobenzo[b][1,4]dioxindiyl, each optionally substituted with one or more substituents Q. In certain embodiments, L² is piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]dioxol-2,5-diyl, or 2,3-dihydrobenzo[b][1,4]dioxin-2,6-diyl, each optionally substituted with one or more substituents Q. In certain embodiments, L² is phen-1,2-diyl, phen-1,3-diyl, phen-1,4-diyl, 4-methoxyphen-1,3-diyl, 2-cyanophen-1,4-diyl, 2-fluorophen-1,4-diyl, 2-chlorophen-1,4-diyl, 2-hydroxyphen-1,4-diyl, 2,3-dihydroinden-1,4-diyl, 2,3-dihydroinden-2,5-diyl, naphth-1,5-diyl, naphth-2,6-diyl, pyrazol-1,3-diyl, pyrazol-1,4-diyl, pyridin-2,3-diyl, pyridin-2,5-diyl, indol-2,5-diyl, indazol-3,7-diyl, benzothiazol-2,6-diyl, quinol-2,6-diyl, quinol-3,7-diyl, piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]dioxol-2,5-diyl, or 2,3-dihydrobenzo[b][1,4]dioxin-2,6-diyl.

In certain embodiments, U is —C(R^{2a})═, wherein R^{2a} is as defined herein. In certain embodiments, U is —C(H)═. In certain embodiments, U is —C(R^{2a})═, wherein R² is methyl or phenyl, each optionally substituted with one or more substituents Q. In certain embodiments, U is —N═. In certain embodiments, U is —N(R^{2b})—, wherein R^{2b} is as defined herein. In certain embodiments, U is —N(H)—. In certain embodiments, U is —N(CH₃)—. In certain embodiments, U is —O—. In certain embodiments, U is —S—. In certain embodiments, U is -A-L¹-L²-R³, wherein A, L¹, L², and R³ are each as defined herein.

In certain embodiments, V is —C(R^{2a})═, wherein R^{2a} is as defined herein. In certain embodiments, V is —C(H)═. In certain embodiments, V is —C(R^{2a})═, wherein R^{2a} is methyl or phenyl, each optionally substituted with one or more substituents Q. In certain embodiments, V is —N═. In certain embodiments, V is —N(R^{2b})—, wherein R² is as defined herein. In certain embodiments. V is —N(H)—. In certain embodiments, V is —N(CH₃)—. In certain embodiments, V is —O—. In certain embodiments, V is —S—. In certain embodiments. V is -A-L¹-L²-R³, wherein A, L¹, L², and R³ are each as defined herein.

In certain embodiments, X is —C(R^{2a})═, wherein R^{2a} is as defined herein. In certain embodiments, X is —C(H)═. In certain embodiments, X is —C(R^{2a})═, wherein R^{2a} is methyl or phenyl, each optionally substituted with one or more substituents Q. In certain embodiments, X is —N═. In certain embodiments, X is —N(R^{2b})═, wherein R^{2b} is as defined herein. In certain embodiments, X is —N(H)—. In certain embodiments, X is —N(CH₃)—. In certain embodiments, X is —O—. In certain embodiments, X is —S—. In certain embodiments, X is -A-L¹-L²-R³, wherein A, L¹, L², and R³ are each as defined herein.

In certain embodiments, Y is a bond. In certain embodiments, Y is —C(R^{2a}r, wherein R^{2a} is as defined herein. In certain embodiments, Y is —C(H)═. In certain embodiments, Y is —C(R^{2a})═, wherein R^{2a} is methyl or phenyl, each optionally substituted with one or more substituents Q. In certain embodiments, Y is —C(R^{2a})═, wherein R^{2a} is phenyl, optionally substituted with one or more substituents Q. In certain embodiments, Y is —N═.

In certain embodiments, Z is —C(R^{2a})═, wherein R^{2a} is as defined herein. In certain embodiments, Z is —C(H)═. In certain embodiments, Z is —C(R^{2a})═, wherein R^{2a} is methyl or phenyl, each optionally substituted with one or more substituents Q. In certain embodiments, Z is —N═. In certain embodiments, Z is —N(R^{2b})—, wherein R^{2b} is as defined herein. In certain embodiments, Z is —N(H)—. In certain embodiments, Z is —N(CH₃)—. In certain embodiments, Z is —O—. In certain embodiments, Z is —S—. In certain embodiments, Z is -A-L¹-L²-R³, wherein A, L¹, L², and R³ are each as defined herein.

In certain embodiments, m is an integer of 0. In certain embodiments, m is an integer of 1. In certain embodiments, m is an integer of 2. In certain embodiments, m is an integer of 3. In certain embodiments, m is an integer of 4.

In certain embodiments, n is an integer of 0. In certain embodiments, n is an integer of 1. In certain embodiments, n is an integer of 2. In certain embodiments, n is an integer of 3. In certain embodiments, n is an integer of 4. In certain embodiments, n is an integer of 5. In certain embodiments, n is an integer of 6.

In certain embodiments, p is an integer of 0. In certain embodiments, p is an integer of 1. In certain embodiments, p is an integer of 2. In certain embodiments, p is an integer of 3. In certain embodiments, p is an integer of 4.

In one embodiment, provided herein is a compound of:
1-(4-(1,1-dioxidobenzo[b]thiophen-7-yl)benzyl)-3-(2-ethynylthiazol-4-yl)urea A1;
1-(2-ethynylthiazol-4-yl)-3-(4-(3-hydroxy-1,1-dioxido-2,3-dihydrobenzo[b]-thiophen-7-yl)benzyl)urea A2;
1-(4-(1,1-dioxido-3-oxo-2,3-dihydrobenzo[b]thiophen-7-yl)benzyl)-3-(2-ethynylthiazol-4-yl)urea A3;
1-(2-ethynylthiazol-4-yl)-3-(4-(8-oxo-7,8-dihydro-6H-thiazolo[5,4-e]isoindol-5-yl)-benzyl)urea A4;
(S)-1-(2-ethynylthiazol-4-yl)-3-((3′-(3-hydroxypyrrolidin-1-yl)-[1,1′-biphenyl]-4-yl)-methyl)urea A5;
1-(4-(benzo[d]thiazol-7-yl)benzyl)-3-(2-ethynylthiazol-4-yl)urea A6;
1-(2-ethynylthiazol-4-yl)-3-((3′-(pyrrolidin-1-yl)-[1,1′-biphenyl]-4-yl)methyl)urea A7;
1-([1,1′-biphenyl]-4-ylmethyl)-3-(2-ethynylthiazol-4-yl)urea A8;
1-(2-ethynylthiazol-4-yl)-3-(4-(4-hydroxyquinazolin-5-yl)benzyl)urea A9;
4′-((3-(2-ethynylthiazol-4-yl)ureido)methyl)-[1,1′-biphenyl]-3-carboxamide A10;
1-(2-ethynylthiazol-4-yl)-3-(4-(3-methyl-4-oxo-3,4-dihydroquinazolin-5-yl)benzyl)urea A11;
1-(2-ethynylthiazol-4-yl)-3-(4-(6-fluorobenzo[d]thiazol-5-yl)benzyl)urea A12;
4′-((3-(2-ethynylthiazol-4-yl)ureido)methyl)-N-methyl-[1,1′-biphenyl]-2-sulfonamide A13:
4′-((3-(2-ethynylthiazol-4-yl)ureido)methyl)-N-methyl-[1,1′-biphenyl]-3-carboxamide A14:
1-(4-(1,5-dimethyl-1H-indazol-4-yl)benzyl)-3-(2-ethynylthiazol-4-yl)urea A15;
1-(2-ethynylthiazol-4-yl)-3-(4-(1-methyl-1H-indazol-4-yl)benzyl)urea A16:
7-(4-((3-(2-ethynylthiazol-4-yl)ureido)methyl)phenyl)-N-methylbenzo[d]thiazole-6-carboxamide A17;
1-(2-ethynylthiazol-4-yl)-3-(4-(1-hydroxyisoquinolin-8-yl)benzyl)urea A18:
7-(4-((3-(2-ethynylthiazol-4-yl)ureido)methyl)phenyl)benzo[d]thiazole-6-carboxamide A19:
4-(4-((3-(2-ethynylthiazol-4-yl)ureido)methyl)phenyl)-1-methyl-1H-indazole-6-carboxamide A20;
4-(4-((3-(2-ethynylthiazol-4-yl)ureido)methyl)phenyl)-N,1-dimethyl-1H-indazole-6-carboxamide A21;
methyl 4-(4-((3-(2-ethynylthiazol-4-yl)ureido)methyl)phenyl)-1-methyl-1H-indazole-6-carboxylate A22; or
4-(4-((3-(2-ethynylthiazol-4-yl)ureido)methyl)phenyl)-N,N,1-trimethyl-1H-indazole-6-carboxamide A23;
1-(2-ethynylthiazol-4-yl)-3-(4-(1-methyl-3-oxo-2,3-dihydro-1H-indazol-4-yl)benzyl)urea A24;
(S)-1-(1-(4-(benzo[d]thiazol-7-yl)phenyl)ethyl)-3-(2-ethynylthiazol-4-yl)urea A25;
(R)-1-(1-(4-(benzo[d]thiazol-7-yl)phenyl)ethyl)-3-(2-ethynylthiazol-4-yl)urea A26;
1-(4-(2,2-difluoro-1,1-dioxido-3-oxo-2,3-dihydrobenzo[b]thiophen-7-yl)benzyl)-3-(2-ethynylthiazol-4-yl)urea A27;
1-(2-ethynylthiazol-4-yl)-3-((3′-(oxetan-3-ylamino)-[1,1′-biphenyl]-4-yl)methyl)urea A28;
1-((2′-cyano-[1,1′-biphenyl]-4-yl)methyl)-3-(2-ethynylthiazol-4-yl)urea A29;
1-(2-ethynylthiazol-4-yl)-3-(4-(pyrrolidin-1-yl)benzyl)urea A30:
1-(4-((3-(2-ethynylthiazol-4-yl)ureido)methyl)phenyl)pyrrolidine-2-carboxamide A31;
(S)-1-(4-(2-cyanopyrrolidin-1-yl)benzyl)-3-(2-ethynylthiazol-4-yl)urea A32;
(R)-1-(4-(2-cyanopyrrolidin-1-yl)benzyl)-3-(2-ethynylthiazol-4-yl)urea A33:
1-(2-(3-(2-cyanophenyl)azetidin-1-yl)-2-oxoethyl)-3-(2-ethynylthiazol-4-yl)urea A34;
1-(2-(4-(2-cyanophenyl)piperidin-1-yl)-2-oxoethyl)-3-(2-ethynylthiazol-4-yl)urea A35;
1-(2-(4-(2-cyanophenyl)piperazin-1-yl)-2-oxoethyl)-3-(2-ethynylthiazol-4-yl)urea A36;
1-((1-(2-cyanophenyl)piperidin-4-yl)methyl)-3-(2-ethynylthiazol-4-yl)urea A37;
1-(adamantan-1-ylmethyl)-3-(2-ethynylthiazol-4-yl)urea A38;
1-(1-(4-(benzo[d]thiazol-7-yl)phenyl)piperidin-4-yl)-3-(2-ethynylthiazol-4-yl)urea A39;
(R)-1-(1-(4-(benzo[d]thiazol-7-yl)phenyl)-2-hydroxyethyl)-3-(2-ethynylthiazol-4-yl)urea A40;
(S)-1-(1-(4-(benzo[d]thiazol-7-yl)phenyl)-2-hydroxyethyl)-3-(2-ethynylthiazol-4-yl)urea A41;
(R)-1-(2-ethynylthiazol-4-yl)-3-(2-hydroxy-1-(3′-(pyrrolidin-1-yl)-[1,1′-biphenyl]-4-yl)-ethyl)urea A42;
(R)-2-(4-(2-(1-cyanocyclopropyl)pyridin-3-yl)phenyl)-2-(3-(2-ethynylthiazol-4-yl)-ureido)-N-methylacetamide A43;
(R)-1-(1-(2′-cyano-[1,1′-biphenyl]-4-yl)-2-hydroxyethyl)-3-(2-ethynylthiazol-4-yl)urea A44;
(R)-1-(1-(5-(2-cyanophenyl)pyridin-2-yl)-2-hydroxyethyl)-3-(2-ethynylthiazol-4-yl)urea A45;
(R)-2-(2′-cyano-[1,1′-biphenyl]-4-yl)-2-(3-(2-ethynylthiazol-4-yl)ureido)ethyl carbamate A46;
(R)-2-(4-(3-cyanopyridin-2-yl)phenyl)-2-(3-(2-ethynylthiazol-4-yl)ureido)ethyl carbamate A47;
(R)-2-(4-(4-cyanopyridin-2-yl)phenyl)-2-(3-(2-ethynylthiazol-4-yl)ureido)ethyl carbamate A48:
(R)-2-(3-(2-ethynylthiazol-4-yl)ureido)-2-(4-(isoquinolin-8-yl)phenyl)ethyl carbamate A49;
(R)-2-(3-(2-ethynylthiazol-4-yl)ureido)-2-(4-(quinazolin-8-yl)phenyl)ethyl carbamate A50;
(R)-2-(3-(2-ethynylthiazol-4-yl)ureido)-2-(4-(quinoxalin-5-yl)phenyl)ethyl carbamate A51;
(R)-1-(1-(4-(7-cyanoquinolin-8-yl)phenyl)-2-hydroxyethyl)-3-(2-ethynylthiazol-4-yl)urea A52;
(R)-1-(1-(4-(3-cyano-1-methyl-1H-indazol-4-yl)phenyl)-2-hydroxyethyl)-3-(2-ethynyl-thiazol-4-yl)urea A53;
(R)-1-(1-(2′-(1-cyanocyclopropyl)-[1,1′-biphenyl]-4-yl)-2-hydroxyethyl)-3-(2-ethynyl-thiazol-4-yl)urea A54:
(R)-2-(2′-(1-cyanocyclopropyl)-[1,1′-biphenyl]-4-yl)-2-(3-(2-ethynylthiazol-4-yl)ureido)-ethyl carbamate A55;
(R)-2-(4-(6-(1-cyanocyclopropyl)pyridin-2-yl)phenyl)-2-(3-(2-ethynylthiazol-4-yl)-ureido)ethyl carbamate A56;
(R)-2-(4-(4-(1-cyanocyclopropyl)pyridin-2-yl)phenyl)-2-(3-(2-ethynylthiazol-4-yl)-ureido)ethyl carbamate A57;
(R)-2-(4-(3-(1-cyanocyclopropyl)pyridin-2-yl)phenyl)-2-(3-(2-ethynylthiazol-4-yl)-ureido)ethyl carbamate A58;
(R)-1-(1-(6-(2-(1-cyanocyclopropyl)phenyl)pyridin-3-yl)-2-hydroxyethyl)-3-(2-ethynyl-thiazol-4-yl)urea A59;
(R)-2-(5-(2-(1-cyanocyclopropyl)phenyl)pyridin-2-yl)-2-(3-(2-ethynylthiazol-4-yl)-ureido)ethyl carbamate A60;
(S)-2-(5-(2-(1-cyanocyclopropyl)phenyl)pyridin-2-yl)-2-(3-(2-ethynylthiazol-4-yl)-ureido)ethyl carbamate A61:
(R)-1-(1-(4-(2-(1-cyanocyclopropyl)pyridin-3-yl)phenyl)-2-hydroxyethyl)-3-(2-ethynyl-thiazol-4-yl)urea A62;
(R)-2-(4-(2-(1-cyanocyclopropyl)pyridin-3-yl)phenyl)-2-(3-(2-ethynylthiazol-4-yl)-ureido)ethyl carbamate A63:
(R)-1-(1-(4-(2-(1-cyanocyclopropyl)-5-fluoropyridin-3-yl)phenyl)-2-hydroxy-ethyl)-3-(2-ethynylthiazol-4-yl)urea A64;
(R)-1-(1-(3-chloro-4-(2-(1-cyanocyclopropyl)pyridin-3-yl)phenyl)-2-hydroxy-ethyl)-3-(2-ethynylthiazol-4-yl)urea A65;
(S)-1-(1-(3-chloro-4-(2-(1-cyanocyclopropyl)pyridin-3-yl)phenyl)-2-hydroxy-ethyl)-3-(2-ethynylthiazol-4-yl)urea A66;
(R)-1-(1-(3-chloro-4-(2-(1-cyanocyclopropyl)pyridin-3-yl)phenyl)-2-hydroxy-ethyl)-3-(2-ethynylthiazol-4-yl)-1-methylurea A67;
(R)-1-(1-(4-(2-(1-cyanocyclopropyl)pyridin-3-yl)-3-fluorophenyl)-2-hydroxy-ethyl)-3-(2-ethynylthiazol-4-yl)urea A68;
(R)-2-(3-chloro-4-(2-(1-cyanocyclopropyl)pyridin-3-yl)phenyl)-2-(3-(2-ethynyl-thiazol-4-yl)ureido)ethyl carbamate A69;
(R)-2-(3-chloro-4-(2-(1-cyanocyclopropyl)pyridin-3-yl)phenyl)-2-(3-(2-ethynyl-thiazol-4-yl)-1-methylureido)ethyl carbamate A70;
(R)-1-(1-(3-chloro-4-(2-(1-cyanocyclopropyl)pyridin-3-yl)phenyl)-2-hydroxy-ethyl)-3-(5-ethynyl-1,3,4-thiadiazol-2-yl)urea A71;
(R)-1-(1-(3-chloro-4-(2-(1-cyanocyclopropyl)pyridin-3-yl)phenyl)-2-(methyl-sulfonyl)-ethyl)-3-(2-ethynylthiazol-4-yl)urea A72:
(R)-2-(3-chloro-4-(2-(1-cyanocyclopropyl)pyridin-3-yl)phenyl)-2-(3-(2-ethynyl-thiazol-4-yl)ureido)ethanesulfonamide A73;
(R)-2-(3-chloro-4-(2-(1-cyanocyclopropyl)pyridin-3-yl)phenyl)-2-(3-(2-ethynyl-thiazol-4-yl)ureido)-N-methylacetamide A74;
(R)-1-(1-(5-(2-(1-cyanocyclopropyl)-4-fluorophenyl)pyridin-2-yl)-2-hydroxy-ethyl)-3-(2-ethynylthiazol-4-yl)urea A75;
(R)-2-(5-(2-(1-cyanocyclopropyl)-4-fluorophenyl)pyridin-2-yl)-2-(3-(2-ethynyl-thiazol-4-yl)ureido)ethyl carbamate A76:
(R)-1-(1-(5-(2-(1-cyanocyclopropyl)-5-fluorophenyl)pyridin-2-yl)-2-hydroxyethyl)-3-(2-ethynylthiazol-4-yl)urea A77;
(R)-1-(1-(5-(2-(1-cyanocyclopropyl)-6-fluorophenyl)pyridin-2-yl)-2-hydroxy-ethyl)-3-(2-ethynylthiazol-4-yl)urea A78;
(R)-1-(1-(5-(2-(1-cyanocyclopropyl)-4,6-difluorophenyl)pyridin-2-yl)-2-hydroxyethyl)-3-(2-ethynylthiazol-4-yl)urea A79;
1-((1R)-1-(5-(2-(2,2-difluorocyclopropyl)phenyl)pyridin-2-yl)-2-hydroxyethyl)-3-(2-ethynylthiazol-4-yl)urea A80;
1-((1R)-1-(5-(2-(2,2-difluorocyclopropyl)-4-fluorophenyl)pyridin-2-yl)-2-hydroxyethyl)-3-(2-ethynylthiazol-4-yl)urea A81:
1-((1R)-1-(5-(2-(2,2-difluorocyclopropyl)-4-fluorophenyl)pyridin-2-yl)-2-hydroxyethyl)-3-(2-ethynylthiazol-4-yl)-1-methylurea A82:
(2R)-2-(5-(2-(2,2-difluorocyclopropyl)-4-fluorophenyl)pyridin-2-yl)-2-(3-(2-ethynyl-thiazol-4-yl)ureido)ethyl carbamate A83;
(2R)-2-(5-(2-(2,2-difluorocyclopropyl)-4-fluorophenyl)pyridin-2-yl)-2-(3-(2-ethynyl-thiazol-4-yl)-1-methylureido)ethyl carbamate A84;
1-((1R)-1-(5-(2-(2,2-difluorocyclopropyl)-4-fluorophenyl)pyridin-2-yl)-2-hydroxyethyl)-3-(5-ethynyl-1,3,4-thiadiazol-2-yl)urea A85:
1-((1R)-1-(5-(2-(2,2-difluorocyclopropyl)-4-fluorophenyl)pyridin-2-yl)-2-hydroxyethyl)-3-(5-ethynyl-1,3,4-thiadiazol-2-yl)-1-methylurea A86;
(2R)-2-(5-(2-(2,2-difluorocyclopropyl)-4-fluorophenyl)pyridin-2-yl)-2-(3-(5-ethynyl-1,3,4-thiadiazol-2-yl)ureido)ethyl carbamate A87:
(R)-1-(2-ethynylthiazol-4-yl)-3-(2-hydroxy-1-(4-(6-(pyrrolidin-1-yl)pyridin-2-yl)phenyl)-ethyl)urea A88;
(R)-1-(2-ethynylthiazol-4-yl)-3-(2-hydroxy-1-(6-(pyrrolidin-1-yl)-[2,3′-bipyridin]-6′-yl)-ethyl)urea A89;
(R)-2-(3-(2-ethynylthiazol-5-yl)ureido)-2-(6-(pyrrolidin-1-yl)-[2,3′-bipyridin]-6′-yl)ethyl carbamate A90;
(R)-3-(2-ethynylthiazol-4-yl)-1-(2-hydroxy-1-(6-(pyrrolidin-1-yl)-[2,3′-bipyridin]-6′-yl)-ethyl)-1-methylurea A91;
(R)-2-(3-(2-ethynylthiazol-4-yl)-1-methylureido)-2-(6-(pyrrolidin-1-yl)-[2,3′-bipyridin]-6′-yl)ethyl carbamate A92;
(R)-1-(5-ethynyl-1,3,4-thiadiazol-2-yl)-3-(2-hydroxy-1-(4-(6-(pyrrolidin-1-yl)-pyridin-2-yl)phenyl)ethyl)urea A93;
(R)-2-(3-(5-ethynyl-1,3,4-thiadiazol-2-yl)ureido)-2-(4-(6-(pyrrolidin-1-yl)-pyridin-2-yl)-phenyl)ethyl carbamate A94:
(R)-2-(3-(5-ethynyl-1,3,4-thiadiazol-2-yl)-1-methylureido)-2-(4-(6-(pyrrolidin-1-yl)-pyridin-2-yl)phenyl)ethyl carbamate A95:
(R)-1-(5-ethynyl-1,3,4-thiadiazol-2-yl)-3-(2-hydroxy-1-(6-(pyrrolidin-1-yl)-[2,3′-bipyridin]-6′-yl)ethyl)urea A96;
(R)-2-(3-(5-ethynyl-1,3,4-thiadiazol-2-yl)ureido)-2-(6-(pyrrolidin-1-yl)-[2,3′-bipyridin]-6′-yl)ethyl carbamate A97:
(R)-1-(1-(4-(2-(dimethylamino)pyridin-3-yl)phenyl)-2-hydroxyethyl)-3-(2-ethynyl-thiazol-4-yl)urea A98;
(R)-2-(4-(2-(dimethylamino)pyridin-3-yl)phenyl)-2-(3-(2-ethynylthiazol-4-yl)-ureido)-ethyl carbamate A99:
(R)-1-(1-(3-chloro-4-(2-(dimethylamino)pyridin-3-yl)phenyl)-2-hydroxyethyl)-3-(2-ethynylthiazol-4-yl)urea A100;
(R)-2-(3-chloro-4-(2-(dimethylamino)pyridin-3-yl)phenyl)-2-(3-(2-ethynyl-thiazol-4-yl)-ureido)ethyl carbamate A101;
(R)-1-(1-(4-(2-(3,3-difluoroazetidin-1-yl)pyridin-3-yl)phenyl)-2-hydroxyethyl)-3-(2-ethynylthiazol-4-yl)urea A102;
(R)-2-(4-(2-(3,3-difluoroazetidin-1-yl)pyridin-3-yl)phenyl)-2-(3-(2-ethynyl-thiazol-4-yl)-ureido)ethyl carbamate A103;
(R)-1-(2-ethynylthiazol-4-yl)-3-(2-hydroxy-1-(4-(1-hydroxyisoquinolin-8-yl)-phenyl)-ethyl)urea A104:
(R)-2-(3-(2-ethynylthiazol-4-yl)ureido)-2-(4-(1-hydroxyisoquinolin-8-yl)phenyl)-ethyl carbamate A105;
(R)-1-(2-ethynylthiazol-4-yl)-3-(2-hydroxy-1-(5-(1-hydroxyisoquinolin-8-yl)-pyridin-2-yl)ethyl)urea A106;
(R)-1-(1-(3-chloro-4-(1-hydroxyisoquinolin-8-yl)phenyl)-2-hydroxyethyl)-3-(2-ethynyl-thiazol-4-yl)urea A107:
(R)-1-(5-ethynyl-1,3,4-thiadiazol-2-yl)-3-(2-hydroxy-1-(4-(1-hydroxyisoquinolin-8-yl)-phenyl)ethyl)urea A108;
(R)-1-(2-ethynylthiazol-4-yl)-3-(2-hydroxy-1-(4-(4-hydroxyquinazolin-5-yl)phenyl)-ethyl)urea A109;
(R)-1-(1-(3-chloro-4-(4-hydroxyquinazolin-5-yl)phenyl)-2-hydroxyethyl)-3-(2-ethynyl-thiazol-4-yl)urea A110;
(R)-1-(1-(4-(benzo[d]thiazol-7-yl)phenyl)-2-hydroxyethyl)-3-(2-ethynylthiazol-4-yl)-1-methylurea A111;
(R)-3-(1-(4-(benzo[d]thiazol-7-yl)phenyl)-2-hydroxyethyl)-1-(2-ethynylthiazol-4-yl)-1-methylurea A112;
(R)-1-(1-(4-(benzo[d]thiazol-7-yl)phenyl)-2-hydroxyethyl)-3-(2-ethynylthiazol-4-yl)-1,3-dimethylurea A113:
(R)-1-(1-(5-(benzo[d]thiazol-7-yl)pyridin-2-yl)-2-hydroxyethyl)-3-(2-ethynyl-thiazol-4-yl)urea A114;
(R)-2-(3-chloro-4-(4-fluorobenzo[d]thiazol-7-yl)phenyl)-2-(3-(2-ethynylthiazol-4-yl)-ureido)ethane-1-sulfonamide A115;
(R)-2-(4-(benzo[d]thiazol-7-yl)phenyl)-2-(3-(2-ethynylthiazol-4-yl)ureido)ethyl L-valinate A116;
(5Z,8Z,11Z,14Z)—(R)-2-(4-(benzo[d]thiazol-7-yl)phenyl)-2-(3-(2-ethynylthiazol-4-yl)-ureido)ethyl icosa-5,8,11,14-tetraenoate A117;
(R)-1-(1-(4-(Benzo[d]thiazol-7-yl)phenyl)-2-hydroxyethyl)-3-(2-ethynylthiazol-5-yl)urea A118;
(R)-1-(1-(4-(Benzo[d]thiazol-7-yl)phenyl)-2-hydroxyethyl)-3-(2-ethynyl-5-methyl-thiazol-4-yl)urea A119:
(R)-1-(1-(4-(Benzo[d]thiazol-7-yl)phenyl)-2-hydroxyethyl)-3-(5-ethynyl-1,3,4-thiadiazol-2-yl)urea A120;
(R)-1-(1-(4-(Benzo[d]thiazol-7-yl)phenyl)-2-hydroxyethyl)-3-(3-ethynyl-1,2,4-thiadiazol-5-yl)urea A121;
(R)-1-(1-(4-(Benzo[d]thiazol-7-yl)phenyl)-2-hydroxyethyl)-3-(5-ethynyl-1,2,4-thiadiazol-3-yl)urea A122;
(R)-1-(4-ethynylpyrimidin-2-yl)-3-(2-hydroxy-1-(4-(6-(pyrrolidin-1-yl)pyridin-2-yl)-phenyl)ethyl)urea A123:
(R)-1-(6-ethynylpyridin-2-yl)-3-(2-hydroxy-1-(4-(6-(pyrrolidin-1-yl)pyridin-2-yl)-phenyl)ethyl)urea A124:
(R)-1-(2-ethynylpyrimidin-4-yl)-3-(2-hydroxy-1-(4-(6-(pyrrolidin-1-yl)pyridin-2-yl)-phenyl)ethyl)urea A125:
(R)-1-(1-(4-(3,3-difluoroazetidin-1-yl)phenyl)-2-hydroxyethyl)-3-(2-ethynyl-thiazol-4-yl)urea A126:
(R)-2-(4-(3,3-difluoroazetidin-1-yl)phenyl)-2-(3-(2-ethynylthiazol-4-yl)ureido)-ethyl carbamate A127;
(R)-1-(2-ethynylthiazol-4-yl)-3-(2-hydroxy-1-(4-(pyrrolidin-1-yl)phenyl)ethyl)-urea A128:
(R)-1-(2-ethynylthiazol-4-yl)-3-(2-hydroxy-1-(4-(2-oxopyrrolidin-1-yl)phenyl)-ethyl)urea A129;
(R)-1-(1-(4-(3,3-difluoropyrrolidin-1-yl)phenyl)-2-hydroxyethyl)-3-(2-ethynyl-thiazol-4-yl)urea A130;
(R)-1-(2-ethynylthiazol-4-yl)-3-(2-hydroxy-1-(4-(2-oxopiperidin-1-yl)phenyl)-ethyl)urea A131;
(R)-1-(2-ethynylthiazol-4-yl)-3-(2-hydroxy-1-(4-(2-oxopyridin-1(2H)-yl)phenyl)-ethyl)-urea A132;
(R)-1-(2-ethynylthiazol-4-yl)-3-(2-hydroxy-1-(4-morpholinophenyl)ethyl)urea A133:
(R)-1-(2-ethynylthiazol-4-yl)-3-(2-hydroxy-1-(4-(3-oxomorpholino)phenyl)-ethyl)urea A134;
(R)-1-(2-ethynylthiazol-4-yl)-3-(2-hydroxy-1-(5-(piperidin-1-yl)pyridin-2-yl)-ethyl)urea A135;
(R)-1-(1-(5-(3,3-difluoropiperidin-1-yl)pyridin-2-yl)-2-hydroxyethyl)-3-(2-ethynyl-thiazol-4-yl)urea A136;
(R)-1-(1-(4-(azepan-1-yl)phenyl)-2-hydroxyethyl)-3-(2-ethynylthiazol-4-yl)urea A137;
(R)-1-(2-ethynylthiazol-4-yl)-3-(2-hydroxy-1-(4-(2-oxoazepan-1-yl)phenyl)-ethyl)urea A138;
(R)-1-(2-ethynylthiazol-4-yl)-3-(2-hydroxy-1-(4-(1-methyl-1,5,6,7-tetrahydro-4H-pyrazolo[4,3-b]pyridin-4-yl)phenyl)ethyl)urea A139;
(R)-1-(2-ethynylthiazol-4-yl)-3-(2-hydroxy-1-(4-(2-methyl-2,5,6,7-tetrahydro-4H-pyrazolo[4,3-b]pyridin-4-yl)phenyl)ethyl)urea A140;
(R)-1-(1-(6′-cyano-2′,3′,4′,5′-tetrahydro-[1,1′-biphenyl]-4-yl)-2-hydroxyethyl)-3-(2-ethynylthiazol-4-yl)urea A141;
(R)-1-(1-(5-(2-cyanocyclohex-1-en-1-yl)pyridin-2-yl)-2-hydroxyethyl)-3-(2-ethynyl-thiazol-4-yl)urea A142;
1-((1R)-1-(4-(1-acetylpiperidin-2-yl)phenyl)-2-hydroxyethyl)-3-(2-ethynyl-thiazol-4-yl)-urea A143;
(R)-1-(1-(3-(2-cyanophenyl)azetidin-1-yl)-3-hydroxy-1-oxopropan-2-yl)-3-(2-ethynyl-thiazol-4-yl)urea A144;
(S)-1-(1-(3-(2-cyanophenyl)azetidin-1-yl)-3-hydroxy-1-oxopropan-2-yl)-3-(2-ethynyl-thiazol-4-yl)urea A145;
(R)-1-(1-(4-(2-cyanophenyl)piperidin-1-yl)-3-hydroxy-1-oxopropan-2-yl)-3-(2-ethynyl-thiazol-4-yl)urea A146;
(S)-1-(1-(4-(2-cyanophenyl)piperidin-1-yl)-3-hydroxy-1-oxopropan-2-yl)-3-(2-ethynyl-thiazol-4-yl)urea A147;
(R)-1-(1-(4-(2-cyanophenyl)piperazin-1-yl)-3-hydroxy-1-oxopropan-2-yl)-3-(2-ethynyl-thiazol-4-yl)urea A148;
(S)-1-(1-(4-(2-cyanophenyl)piperazin-1-yl)-3-hydroxy-1-oxopropan-2-yl)-3-(2-ethynyl-thiazol-4-yl)urea A149;
(R)-1-(1-(1-(2-cyanophenyl)piperidin-4-yl)-2-hydroxyethyl)-3-(2-ethynylthiazol-4-yl)-urea A150;
(S)-1-(1-(1-(2-cyanophenyl)piperidin-4-yl)-2-hydroxyethyl)-3-(2-ethynylthiazol-4-yl)-urea A151;
(S)-1-(2-ethynylthiazol-4-yl)-3-(3-hydroxy-1-oxo-1-(6-azaspiro[2.5]octan-6-yl)propan-2-yl)urea A152;
(S)-1-(2-ethynylthiazol-4-yl)-3-(3-hydroxy-1-oxo-1-(7-azaspiro[3.5]nonan-7-yl)propan-2-yl)urea A153;
(S)-1-(2-ethynylthiazol-4-yl)-3-(3-hydroxy-1-oxo-1-(8-azaspiro[4.5]decan-8-yl)propan-2-yl)urea A154;
(R)-1-(2-ethynylthiazol-4-yl)-3-(3-hydroxy-1-oxo-1-(8-azaspiro[4.5]decan-8-yl)propan-2-yl)urea A155:
(S)-1-(2-ethynylthiazol-5-yl)-3-(3-hydroxy-1-oxo-1-(1-oxo-8-azaspiro[4.5]decan-8-yl)-propan-2-yl)urea A156:
(S)-1-(2-ethynylthiazol-4-yl)-3-(3-hydroxy-1-oxo-1-(3-azaspiro[5.5]undecan-3-yl)-propan-2-yl)urea A157:
(S)-1-(1-(9,9-difluoro-3-azaspiro[5.5]undecan-3-yl)-3-hydroxy-1-oxopropan-2-yl)-3-(2-ethynylthiazol-4-yl)urea A158;
(S)-1-(1-(4-(3,3-difluoroazetidin-1-yl)piperidin-1-yl)-3-hydroxy-1-oxopropan-2-yl)-3-(2-ethynylthiazol-4-yl)urea A159;
(R)-1-(1-(4-(3,3-difluoroazetidin-1-yl)piperidin-1-yl)-3-hydroxy-1-oxopropan-2-yl)-3-(2-ethynylthiazol-4-yl)urea A160;
(S)-1-(1-(4,4-difluoropiperidin-1-yl)-3-hydroxy-1-oxopropan-2-yl)-3-(2-ethynyl-thiazol-4-yl)urea A161;
1-(6-chloro-8-fluoro-7-(2-fluoro-6-methoxyphenyl)quinazolin-4-yl)-3-(2-ethynylthiazol-4-yl)urea A162;
1-(2-ethynylthiazol-4-yl)-3-(7-(2-fluoro-6-hydroxyphenyl)quinazolin-4-yl)urea A163;
1-(2-ethynylthiazol-4-yl)-3-(7-(2-fluoro-6-methoxyphenyl)quinazolin-4-yl)urea A164;
1-(7-(benzo[d]thiazol-7-yl)-6-chloro-8-fluoroquinazolin-4-yl)-3-(2-ethynyl-thiazol-4-yl)urea A165;
1-(7-(2-(1-cyanocyclopropyl)pyridin-3-yl)quinazolin-4-yl)-3-(2-ethynylthiazol-4-yl)urea A166;
1-((3′-(1-cyanocyclopropyl)-[1,1′-biphenyl]-4-yl)methyl)-3-(2-ethynylthiazol-4-yl)urea A167;
1-(2-ethynylthiazol-4-yl)-3-((3′-(1-hydroxycyclopropyl)-[1,1′-biphenyl]-4-yl)methyl)urea A168;
1-(2-ethynylthiazol-4-yl)-3-((3′-propionyl-[1,1′-biphenyl]-4-yl)methyl)urea A169;
1-(2-ethynylthiazol-4-yl)-3-((3′-(1-hydroxycyclobutyl)-[1,1′-biphenyl]-4-yl)-methyl)urea A170;
1-(4-(2-(3,3-difluoroazetidin-1-yl)-4-hydroxyquinazolin-5-yl)benzyl)-3-(2-ethynyl-thiazol-4-yl)urea A171;
1-(2-ethynylthiazol-4-yl)-3-(4-(4-hydroxy-2-(2-hydroxyethoxy)quinazolin-5-yl)-benzyl)-urea A172;
1-(2-ethynylthiazol-4-yl)-3-(4-(4-(pyrrolidin-1-yl)pyridin-2-yl)benzyl)urea A173;
1-(2-ethynylthiazol-4-yl)-3-(4-(5-(pyrrolidin-1-yl)pyridin-3-yl)benzyl)urea A174;
1-(2-ethynylthiazol-4-yl)-3-(4-(2-(pyrrolidin-1-yl)pyridin-4-yl)benzyl)urea A175;
1-(2-ethynylthiazol-4-yl)-3-(4-(6-(pyrrolidin-1-yl)pyridin-2-yl)benzyl)urea A176;
1-(4-(benzo[d]thiazol-7-yl)-2-cyanobenzyl)-3-(2-ethynylthiazol-4-yl)urea A177;
1-(4-(benzo[d]thiazol-7-yl)-2-cyanobenzyl)-3-(2-ethynylthiazol-4-yl)urea A178;
1-((5-(benzo[d]thiazol-7-yl)pyridin-2-yl)methyl)-3-(2-ethynylthiazol-4-yl)urea A179;
1-((6-(3-(1-cyanocyclopropyl)phenyl)pyridin-3-yl)methyl)-3-(2-ethynylthiazol-4-yl)urea A180;
(R)-1-(1-(3′-(3,3-difluoropyrrolidin-1-yl)-[1,1′-biphenyl]-4-yl)-2-hydroxyethyl)-3-(2-ethynylthiazol-4-yl)urea A181:
(R)-1-(1-(4-cyclohexylphenyl)-2-hydroxyethyl)-3-(2-ethynylthiazol-4-yl)urea A182;
(R)-1-(2-ethynylthiazol-4-yl)-3-(2-hydroxy-1-(2′,3′,4′,5′-tetrahydro-[1,1′-biphenyl]-4-yl)-ethyl)urea A183:
(R)-1-(2-ethynylthiazol-4-yl)-3-(2-hydroxy-1-(4-(1-methylpiperidin-4-yl)phenyl)-ethyl)-urea A184;
(R)-1-(2-ethynylthiazol-4-yl)-3-(2-hydroxy-1-(4-(1-methyl-1,2,3,6-tetrahydro-pyridin-4-yl)phenyl)ethyl)urea A185;
(R)-1-(2-ethynylthiazol-4-yl)-3-(2-hydroxy-1-(3′-(methylsulfonyl)-[1,1′-biphenyl]-4-yl)ethyl)urea A186;
(R)-1-(1-(3′-(1-cyanocyclopropyl)-[1,1′-biphenyl]-4-yl)-2-hydroxyethyl)-3-(2-ethynyl-thiazol-4-yl)urea A187;
1-((1R)-1-(3′-(2,2-difluorocyclopropyl)-[1,1′-biphenyl]-4-yl)-2-hydroxyethyl)-3-(2-ethynylthiazol-4-yl)urea A188;
(R)-1-(2-ethynylthiazol-4-yl)-3-(2-hydroxy-1-(3′-(1-hydroxycyclobutyl)-[1,1′-biphenyl]-4-yl)ethyl)urea A189;
(R)-1-(1-(3′-(azetidin-1-yl)-[1,1′-biphenyl]-4-yl)-2-hydroxyethyl)-3-(2-ethynyl-thiazol-4-yl)urea A190;
(R)-1-(1-(3′-(3,3-difluoroazetidin-1-yl)-[1,1′-biphenyl]-4-yl)-2-hydroxyethyl)-3-(2-ethynylthiazol-4-yl)urea A191;
1-(2-ethynylthiazol-4-yl)-3-((1R)-2-hydroxy-1-(4-(1-methylpiperidin-3-yl)-phenyl)ethyl)-urea A192;
(R)-1-(2-ethynylthiazol-4-yl)-3-(2-hydroxy-1-(4-(1-methyl-1,2,5,6-tetrahydro-pyridin-3-yl)phenyl)ethyl)urea A193:
1-((1R)-1-(4-(1-acetylpiperidin-3-yl)phenyl)-2-hydroxyethyl)-3-(2-ethynyl-thiazol-4-yl)-urea A194;
(R)-1-(2-ethynylthiazol-4-yl)-3-(2-hydroxy-1-(4-(piperidin-1-yl)phenyl)ethyl)-urea A195;
(S)-1-(1-(4-(benzo[d]thiazol-7-yl)phenyl)-2-cyanoethyl)-3-(2-ethynylthiazol-4-yl)urea A196;
(R)-1-(1-(4-(benzo[d]thiazol-7-yl)phenyl)-2-(methylsulfonyl)ethyl)-3-(2-ethynyl-thiazol-4-yl)urea A197;
(R)-2-(4-(benzo[d]thiazol-7-yl)phenyl)-2-(3-(2-ethynylthiazol-4-yl)ureido)-ethane-1-sulfonamide A198:
(R)-2-(4-(benzo[d]thiazol-7-yl)phenyl)-2-(3-(2-ethynylthiazol-4-yl)ureido)-N-methyl-ethane-1-sulfonamide A199;
(R)-1-(1-(4-(benzo[d]thiazol-7-yl)phenyl)-2-methoxyethyl)-3-(2-ethynylthiazol-4-yl)urea A200;
(R)-2-(4-(benzo[d]thiazol-7-yl)phenyl)-2-(3-(2-ethynylthiazol-4-yl)ureido)ethyl acetate A201;
(R)-2-(4-(benzo[d]thiazol-7-yl)phenyl)-2-(3-(2-ethynylthiazol-4-yl)ureido)ethyl pivalate A202;
(R)-1-(1-(4-(benzo[d]thiazol-7-yl)phenyl)-2-hydroxy-2-methylpropyl)-3-(2-ethynyl-thiazol-4-yl)urea A203:
1-((1R)-1-(4-(benzo[d]thiazol-7-yl)phenyl)-2-hydroxypropyl)-3-(2-ethynyl-thiazol-4-yl)-urea A204;
1-(4-(benzo[d]thiazol-7-yl)benzyl)-1-(2-cyanoethyl)-3-(2-ethynylthiazol-4-yl)urea A205:
1-(4-(benzo[d]thiazol-7-yl)benzyl)-3-(2-ethynylthiazol-4-yl)-1-(2-hydroxyethyl)-urea A206:
1-(4-(benzo[d]thiazol-7-yl)benzyl)-3-(2-ethynylthiazol-4-yl)-1-(2-(methylsulfonyl)-ethyl)urea A207:
1-(4-(benzo[d]thiazol-7-yl)benzyl)-1-(cyanomethyl)-3-(2-ethynylthiazol-4-yl)urea A208:
2-(4-(benzo[d]thiazol-7-yl)phenyl)-2-(3-(2-ethynylthiazol-4-yl)ureido)acetamide A209;
(R)-2-(4-(benzo[d]thiazol-7-yl)phenyl)-2-(3-(2-ethynylthiazol-4-yl)ureido)-acetamide A210;
(S)-2-(4-(benzo[d]thiazol-7-yl)phenyl)-2-(3-(2-ethynylthiazol-4-yl)ureido)-acetamide A211;
(R)-2-(3′-(1-cyanocyclopropyl)-[1,1′-biphenyl]-4-yl)-2-(3-(2-ethynylthiazol-4-yl)ureido)-acetamide A212;
(R)-2-(3-(2-ethynylthiazol-4-yl)ureido)-N-methyl-2-(4-(4-oxo-3,4-dihydro-quinazolin-5-yl)phenyl)acetamide A213;
2-(4-(benzo[d]thiazol-7-yl)phenyl)-2-(3-(2-ethynylthiazol-4-yl)ureido)-N-methyl-acetamide A214;
(R)-2-(4-(benzo[d]thiazol-7-yl)phenyl)-2-(3-(2-ethynylthiazol-4-yl)ureido)-N-methyl-acetamide A215;
(S)-2-(4-(benzo[d]thiazol-7-yl)phenyl)-2-(3-(2-ethynylthiazol-4-yl)ureido)-N-methyl-acetamide A216;
(R)-2-(3′-(1-cyanocyclopropyl)-[1,1′-biphenyl]-4-yl)-2-(3-(2-ethynylthiazol-4-yl)ureido)-N-methylacetamide A217;
(R)-2-(3-(2-ethynylthiazol-4-yl)ureido)-2-(3′-(1-hydroxycyclopropyl)-[1,1′-biphenyl]-4-yl)-N-methylacetamide A218;
(R)-2-(3-(2-ethynylthiazol-4-yl)ureido)-N-methyl-2-(3′-propionyl-[1,1′-biphenyl]-4-yl)-acetamide A219;
(R)-2-(4-(benzo[d]thiazol-7-yl)phenyl)-2-(3-(2-ethynylthiazol-4-yl)ureido)-N,N-dimethylacetamide A220;
(R)-2-(3′-cyano-[1,1′-biphenyl]-4-yl)-2-(3-(2-ethynylthiazol-4-yl)ureido)ethyl carbamate A221;
(R)-2-(3′-(1-cyanocyclopropyl)-[1,1′-biphenyl]-4-yl)-2-(3-(2-ethynylthiazol-4-yl)ureido)-ethyl carbonate A222;
(R)-2-(4-(benzo[d]thiazol-7-yl)phenyl)-2-(3-(2-ethynylthiazol-4-yl)ureido)ethyl carbamate A223;
(R)-2-(4-(benzo[d]thiazol-7-yl)phenyl)-2-(3-(2-ethynylthiazol-4-yl)ureido)ethyl methyl-carbamate A224;
(R)-2-(4-(benzo[d][1,2,3]thiadiazol-7-yl)phenyl)-2-(3-(2-ethynylthiazol-4-yl)-ureido)-ethyl carbamate A225;
(R)-2-(4-(benzo[c][1,2,5]thiadiazol-4-yl)phenyl)-2-(3-(2-ethynylthiazol-4-yl)-ureido)-ethyl carbamate A226;
(R)-2-(3-(2-ethynylthiazol-4-yl)ureido)-2-(4-(quinolin-8-yl)phenyl)ethyl carbamate A227:
(R)-2-(3-(2-ethynylthiazol-4-yl)ureido)-2-(4-(isoquinolin-8-yl)phenyl)ethyl carbamate A228:
(R)-2-(3-(2-ethynylthiazol-4-yl)ureido)-2-(4-(isoquinolin-5-yl)phenyl)ethyl carbamate A229:
(R)-2-(3-(2-ethynylthiazol-4-yl)ureido)-2-(4-(quinolin-5-yl)phenyl)ethyl carbamate A230;
(R)-2-(3-(2-ethynylthiazol-4-yl)ureido)-2-(4-(1-methyl-1H-indazol-4-yl)phenyl)-ethyl carbamate A231;
(2R)-2-(3-(2-ethynylthiazol-4-yl)ureido)-2-(4-(1-methyl-4,5,6,7-tetrahydro-1H-indazol-4-yl)phenyl)ethyl carbamate A232;
(R)-2-(3-(2-ethynylthiazol-4-yl)ureido)-2-(4-(1-methyl-6,7-dihydro-1H-indazol-4-yl)-phenyl)ethyl carbamate A233;
(R)-2-(3-(2-ethynylthiazol-4-yl)ureido)-2-(4-(6-(pyrrolidin-1-yl)pyridin-2-yl)-phenyl)-ethyl carbamate A234:
(R)-2-(3-(2-ethynylthiazol-4-yl)ureido)-2-(4-(2-(pyrrolidin-1-yl)pyridin-4-yl)-phenyl)-ethyl carbamate A235:
(R)-2-(5-(3-(1-cyanocyclopropyl)phenyl)pyridin-2-yl)-2-(3-(2-ethynylthiazol-4-yl)-ureido)ethyl carbamate A236;
1-(1-(4-(benzo[d]thiazol-7-yl)phenyl)piperidin-4-yl)-3-(2-ethynylthiazol-4-yl)urea A237:
1-((4-(3-(1-cyanocyclopropyl)phenyl)cyclohexyl)methyl)-3-(2-ethynylthiazol-4-yl)urea A238;
1-((3′-(1-cyanocyclopropyl)-2,3,4,5-tetrahydro-[1,1′-biphenyl]-4-yl)methyl)-3-(2-ethynylthiazol-4-yl)urea A239:
1-((1-(3-(1-cyanocyclopropyl)phenyl)piperidin-4-yl)methyl)-3-(2-ethynylthiazol-4-yl)-urea A240; OR
(R)-1-(1-(4-(benzo[d]thiazol-7-yl)phenyl)-2-hydroxyethyl)-3-(2-ethynyl-pyrimidin-4-yl)-urea A241;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is a compound of:
2-(4-(benzo[d]thiazol-7-yl)phenyl)-N-(2-ethynylthiazol-4-yl)pyrrolidine-1-carboxamide B1;
3-(4-(benzo[d]thiazol-7-yl)phenyl)-N-(2-ethynylthiazol-4-yl)azetidine-1-carboxamide B2;
6-(benzo[d]thiazol-7-yl)-N-(2-ethynylthiazol-4-yl)-3,4-dihydroisoquinoline-2(1H)-carboxamide B3;
5-(benzo[d]thiazol-7-yl)-N-(2-ethynylthiazol-4-yl)isoindoline-2-carboxamide B2:
4-(4-(benzo[d]thiazol-7-yl)phenyl)-N-(2-ethynylthiazol-4-yl)piperazine-1-carboxamide B5:
4-(4-(benzo[d]thiazol-7-yl)phenyl)-N-(2-ethynylthiazol-4-yl)piperidine-1-carboxamide B6;
N-(2-ethynylthiazol-4-yl)-4-(5-(3-(2-oxooxazolidin-3-yl)phenyl)pyridin-2-yl)piperazine-1-carboxamide B7;
N-(2-ethynylthiazol-4-yl)-4-(3′-(oxetan-3-ylamino)-[1,1′-biphenyl]-4-yl)piperazine-1-carboxamide B8;
N-(2-ethynylthiazol-4-yl)-4-(3′-((3-hydroxycyclobutyl)amino)-[1,1′-biphenyl]-4-yl)-piperazine-1-carboxamide B9;
N-(2-ethynylthiazol-4-yl)-4-(3′-(3-hydroxyazetidin-1-yl)-[1,1′-biphenyl]-4-yl)piperazine-1-carboxamide B10;
N-(2-ethynylthiazol-4-yl)-4-(4-(imidazo[1,5-a]pyridin-5-yl)phenyl)piperazine-1-carboxamide B11;
4-(4-([1,2,4]triazolo[4,3-a]pyridin-5-yl)phenyl)-N-(2-ethynylthiazol-4-yl)piperazine-1-carboxamide B12;
N-(2-ethynylthiazol-4-yl)-4-(4-(imidazo[1,5-a]pyridin-8-yl)phenyl)piperazine-1-carboxamide B13;
4-(4-([1,2,3]triazolo[1,5-a]pyridin-4-yl)phenyl)-N-(2-ethynylthiazol-4-yl)piperazine-1-carboxamide B14;
N-(2-ethynylthiazol-4-yl)-4-(3′-(pyrrolidin-1-yl)-[1,1′-biphenyl]-4-yl)piperazine-1-carboxamide B15;
(S)—N-(2-ethynylthiazol-4-yl)-4-(3′-(3-hydroxypyrrolidin-1-yl)-[1,1′-biphenyl]-4-yl)-piperazine-1-carboxamide B16;
4-(4-(1,1-dioxido-3-oxo-2,3-dihydrobenzo[b]thiophen-7-yl)phenyl)-N-(2-ethynylthiazol-4-yl)piperazine-1-carboxamide B17;
N-(2-ethynylthiazol-4-yl)-4-(4-(1-methyl-1H-indazol-4-yl)phenyl)piperazine-1-carboxamide B18;
N-(2-ethynylthiazol-4-yl)-4-(3′-(pyrrolidin-1-yl)-[1,1′-biphenyl]-4-yl)piperazine-1-carboxamide B19;
N-(2-ethynylthiazol-4-yl)-4-(4-(1-oxo-1,2-dihydroisoquinolin-8-yl)phenyl)piperazine-1-carboxamide B20;
N-(2-ethynylthiazol-4-yl)-4-(4-(3-hydroxy-1,1-dioxido-2,3-dihydrobenzo[b]thiophen-7-yl)phenyl)piperazine-1-carboxamide B21;
N-(2-ethynylthiazol-4-yl)-4-(4-(4-oxo-3,4-dihydroquinazolin-5-yl)phenyl)piperazine-1-carboxamide B22;
(S)-4-(4-(benzo[d]thiazol-7-yl)phenyl)-N-(2-ethynylthiazol-4-yl)-2-(hydroxymethyl)-piperazine-1-carboxamide B23;
(R)-4-(4-(benzo[d]thiazol-7-yl)phenyl)-N-(2-ethynylthiazol-4-yl)-2-(hydroxymethyl)-piperazine-1-carboxamide B24;
4-(4-(2-amino-6-cyanobenzo[d]thiazol-7-yl)phenyl)-N-(2-ethynylthiazol-4-yl)piperazine-1-carboxamide B25;
4-(4-(6-cyanobenzo[d]thiazol-7-yl)phenyl)-N-(2-ethynylthiazol-4-yl)piperazine-1-carboxamide B26;
4-(4-(2-aminobenzo[d]thiazol-7-yl)phenyl)-N-(2-ethynylthiazol-4-yl)piperazine-1-carboxamide B27;
4-(4-(benzo[d][1,2,3]thiadiazol-7-yl)phenyl)-N-(2-ethynylthiazol-4-yl)piperazine-1-carboxamide B28;
4-(4-([1,2,4]triazolo[1,5-a]pyridin-8-yl)phenyl)-N-(2-ethynylthiazol-4-yl)piperazine-1-carboxamide B29;
4-(4-([1,2,4]triazolo[4,3-a]pyridin-8-yl)phenyl)-N-(2-ethynylthiazol-4-yl)piperazine-1-carboxamide B30;
4-(2′-cyano-[1,1′-biphenyl]-4-yl)-N-(2-ethynylthiazol-4-yl)piperazine-1-carboxamide B31;
4-(4-([1,2,4]triazolo[4,3-a]pyridin-5-yl)phenyl)-N-(2-ethynylthiazol-4-yl)piperazine-1-carboxamide B32;
N-(2-ethynylthiazol-4-yl)-4-(4-(imidazo[1,2-a]pyridin-5-yl)phenyl)piperazine-1-carboxamide B33;
4-(4-([1,2,4]triazolo[1,5-a]pyridin-5-yl)phenyl)-N-(2-ethynylthiazol-4-yl)piperazine-1-carboxamide B34;
N-(2-ethynylthiazol-4-yl)-4-(4-(imidazo[1,2-a]pyridin-8-yl)phenyl)piperazine-1-carboxamide B35;
(R)-4-(4-(benzo[d]thiazol-7-yl)phenyl)-N-(2-ethynylthiazol-4-yl)-3-(hydroxymethyl)-piperazine-1-carboxamide B36;
(S)-4-(4-(benzo[d]thiazol-7-yl)phenyl)-N-(2-ethynylthiazol-4-yl)-3-(hydroxymethyl)-piperazine-1-carboxamide B37;
4-(3′-(1-cyanocyclopropyl)-[1,1′-biphenyl]-4-yl)-N-(2-ethynylthiazol-4-yl)piperazine-1-carboxamide B38;
4-(3′-cyano-[1,1′-biphenyl]-4-yl)-N-(2-ethynylthiazol-4-yl)piperazine-1-carboxamide B39;
4-(4-(benzo[d]thiazol-7-yl)-2-cyanophenyl)-N-(2-ethynylthiazol-4-yl)piperazine-1-carboxamide B40;
4-(4-(benzo[d]thiazol-7-yl)-3-cyanophenyl)-N-(2-ethynylthiazol-4-yl)piperazine-1-carboxamide B41;
4-(3′-cyclopropyl-[1,1′-biphenyl]-4-yl)-N-(2-ethynylthiazol-4-yl)piperazine-1-carboxamide B42;
N-(2-ethynylthiazol-4-yl)-4-(3′-(2-oxopyrrolidin-1-yl)-[1,1′-biphenyl]-4-yl)piperazine-1-carboxamide B43;
4-((4-(benzo[d]thiazol-7-yl)phenyl)amino)-N-(2-ethynylthiazol-4-yl)piperidine-1-carboxamide B44;
4-(4-(2-amino-[1,2,4]triazolo[1,5-a]pyridin-5-yl)phenyl)-N-(2-ethynylthiazol-4-yl)-piperazine-1-carboxamide B45;
4-(3′-(cyclopent-1-en-1-yl)-[1,1′-biphenyl]-4-yl)-N-(2-ethynylthiazol-4-yl)piperazine-1-carboxamide B46;
N-(2-ethynylthiazol-4-yl)-4-(3′-(2-oxoimidazolidin-1-yl)-[1,1′-biphenyl]-4-yl)piperazine-1-carboxamide B47;
N-(2-ethynylthiazol-4-yl)-4-(3′-(1-hydroxycyclopentyl)-[1,1′-biphenyl]-4-yl)piperazine-1-carboxamide B48;
N-(2-ethynylthiazol-4-yl)-4-(3′-(3-hydroxyazetidin-1-yl)-[1,1′-biphenyl]-4-yl)piperazine-1-carboxamide B49;
N-(2-ethynylthiazol-4-yl)-4-(3′-(3-hydroxypyrrolidin-1-yl)-[1,1′-biphenyl]-4-yl)-piperazine-1-carboxamide B50;
4-(3′-(azetidin-1-yl)-[1,1′-biphenyl]-4-yl)-N-(2-ethynylthiazol-4-yl)piperazine-1-carboxamide B51;
(S)—N-(2-ethynylthiazol-4-yl)-2-(hydroxymethyl)-4-(3′-(pyrrolidin-1-yl)-[1,1′-biphenyl]-4-yl)piperazine-1-carboxamide B52;
(R)-4-(3-cyano-3′-(pyrrolidin-1-yl)-[1,1′-biphenyl]-4-yl)-N-(2-ethynylthiazol-4-yl)-2-(hydroxymethyl)piperazine-1-carboxamide B53:
(R)—N-(2-ethynylthiazol-4-yl)-2-(hydroxymethyl)-4-(3′-(pyrrolidin-1-yl)-[1,1′-biphenyl]-4-yl)piperazine-1-carboxamide B54;
(R)-4-(4-(benzo[d]thiazol-7-yl)phenyl)-N-(2-ethynylthiazol-4-yl)-2-(hydroxymethyl)-piperazine-1-carboxamide B55:
(R)—N-(2-ethynylthiazol-4-yl)-2-(hydroxymethyl)-4-(5-(3-(pyrrolidin-1-yl)phenyl)pyridin-2-yl)piperazine-1-carboxamide B56;
N-(2-ethynylthiazol-4-yl)-4-(5-(3-(2-oxooxazolidin-3-yl)phenyl)pyridin-2-yl)piperazine-1-carboxamide B57;
(R)-4-(5-(benzo[d]thiazol-7-yl)pyridin-2-yl)-N-(2-ethynylthiazol-4-yl)-2-(hydroxymethyl)piperazine-1-carboxamide B58; or
(R)-4-(5-(benzo[d]thiazol-7-yl)-3-cyanopyridin-2-yl)-N-(2-ethynylthiazol-4-yl)-2-(hydroxymethyl)piperazine-1-carboxamide B59:
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a compound of:
2-ethynyl-N-(4-(6-fluorobenzo[d]thiazol-5-yl)phenethyl)thiazole-4-carboxamide C1:
N-(4-(benzo[d]thiazol-7-yl)-3-fluorophenethyl)-2-ethynylthiazole-4-carboxamide C2;
7-(4-(2-(2-ethynylthiazole-4-carboxamido)ethyl)phenyl)benzo[d]thiazole-6-carboxamide C3;
2-ethynyl-N-(2-(3′-(methylcarbamoyl)-[1,1′-biphenyl]-4-yl)ethyl)thiazole-4-carboxamide C4;
2-ethynyl-N-(4-(6-fluorobenzo[d]thiazol-7-yl)phenethyl)thiazole-4-carboxamide C5:
N-(2-(2′-(N,N-dimethylsulfamoyl)-[1,1′-biphenyl]-4-yl)ethyl)-2-ethynylthiazole-4-carboxamide C6;
methyl 2-(2-ethynylthiazole-4-carboxamido)-5-(4-(2-(2-ethynylthiazole-4-carboxamido)-ethyl)phenyl)benzo[d]thiazole-7-carboxylate C7;
7-(4-(2-(2-ethynylthiazole-4-carboxamido)ethyl)phenyl)benzo[d]thiazole-5-carboxamide C8:
7-(4-(2-(2-ethynylthiazole-4-carboxamido)ethyl)phenyl)-N-methylbenzo[d]thiazole-5-carboxamide C9:
methyl 7-(4-(2-(2-ethynylthiazole-4-carboxamido)ethyl)phenyl)benzo[d]thiazole-5-carboxylate C10:
2-ethynyl-N-(4-(quinoxalin-5-yl)phenethyl)thiazole-4-carboxamide C11:
2-ethynyl-N-(2-(2′-(methylcarbamoyl)-[1,1′-biphenyl]-4-yl)ethyl)thiazole-4-carboxamide C12;
2-ethynyl-N-(4-(3-oxoisoindolin-4-yl)phenethyl)thiazole-4-carboxamide C13;
N-(4-(2,3-dioxoindolin-4-yl)phenethyl)-2-ethynylthiazole-4-carboxamide C14;
2-ethynyl-N-(2-(2′-(methylsulfonyl)-[1,1′-biphenyl]-4-yl)ethyl)thiazole-4-carboxamide C15;
2-ethynyl-N-(4-(1-hydroxyisoquinolin-8-yl)phenethyl)thiazole-4-carboxamide C16;
2-ethynyl-N-(4-(1-oxoisoindolin-4-yl)phenethyl)thiazole-4-carboxamide C17;
2-ethynyl-N-(4-(3-oxo-2,3-dihydro-1H-inden-4-yl)phenethyl)thiazole-4-carboxamide C18;
2-ethynyl-N-(4-(thiazolo[4,5-c]pyridin-7-yl)phenethyl)thiazole-4-carboxamide C19′;
2-ethynyl-N-(2-(2′-(N-methylsulfamoyl)-[1,1′-biphenyl]-4-yl)ethyl)thiazole-4-carboxamide C20;
2-ethynyl-N-(4-(isoquinolin-5-yl)phenethyl)thiazole-4-carboxamide C21;
2-ethynyl-N-(4-(2-methyl-1-oxoisoindolin-4-yl)phenethyl)thiazole-4-carboxamide C22;
2-ethynyl-N-(4-(quinolin-5-yl)phenethyl)thiazole-4-carboxamide C23:
2-ethynyl-N-(4-(quinazolin-5-yl)phenethyl)thiazole-4-carboxamide C24;
N-(4-(benzo[d]thiazol-5-yl)phenethyl)-2-ethynylthiazole-4-carboxamide C25;
2-ethynyl-N-(3-(1-methyl-2-oxoindolin-4-yl)phenethyl)thiazole-4-carboxamide C26;
2-ethynyl-N-(3-(2-oxoindolin-4-yl)phenethyl)thiazole-4-carboxamide C27;
N-(4-(1H-indol-4-yl)benzyl)-2-ethynylthiazole-4-carboxamide C28;
2-ethynyl-N-(3-(2-methyl-2H-indazol-4-yl)phenethyl)thiazole-4-carboxamide C29:
2-ethynyl-N-(3-(1-methyl-1H-indazol-4-yl)phenethyl)thiazole-4-carboxamide C30;
N-(4-(benzo[d]thiazol-4-yl)phenethyl)-2-ethynylthiazole-4-carboxamide C31:
2-ethynyl-N-(4-(2-methyl-2H-indazol-4-yl)phenethyl)thiazole-4-carboxamide C32;
2-ethynyl-N-(4-(1-methyl-1H-indazol-4-yl)phenethyl)thiazole-4-carboxamide C33:
N-(4-(benzo[d]thiazol-7-yl)phenethyl)-2-ethynylthiazole-4-carboxamide C34;
2-ethynyl-N-(3-(1-methyl-1H-indol-4-yl)phenethyl)thiazole-4-carboxamide C35;
N-(3-(1H-indol-4-yl)phenethyl)-2-ethynylthiazole-4-carboxamide C36;
2-ethynyl-N-(4-(2-oxoindolin-4-yl)benzyl)thiazole-4-carboxamide C37;
2-ethynyl-N-(3-(pyridin-3-yl)phenethyl)thiazole-4-carboxamide C38;
N-(3-(1H-indazol-4-yl)phenethyl)-2-ethynylthiazole-4-carboxamide C39;
N-(4-(1H-indazol-4-yl)benzyl)-2-ethynylthiazole-4-carboxamide C40;
2-ethynyl-N-(4-(1-methyl-1H-pyrazol-3-yl)phenethyl)thiazole-4-carboxamide C41;
N-(4-(benzo[d]thiazol-6-yl)phenethyl)-2-ethynylthiazole-4-carboxamide C42;
2-ethynyl-N-(4-(1-methyl-2-oxoindolin-4-yl)phenethyl)thiazole-4-carboxamide C43;
2-ethynyl-N-(quinolin-2-ylmethyl)thiazole-4-carboxamide C44;
N-(2-cyanophenethyl)-2-ethynylthiazole-4-carboxamide C45;
methyl 2-(3-((2-ethynylthiazole-4-carboxamido)methyl)phenyl)acetate C46;
N-(3-(2-amino-2-oxoethyl)benzyl)-2-ethynylthiazole-4-carboxamide C47;
2-ethynyl-N-(4-(2-oxoindolin-4-yl)phenethyl)thiazole-4-carboxamide C48;
N-(4-(1H-indazol-4-yl)phenethyl)-2-ethynylthiazole-4-carboxamide C49;
2-ethynyl-N-(4-(1-methyl-1H-indol-4-yl)phenethyl)thiazole-4-carboxamide C50;
N-(4-(1H-indol-4-yl)phenethyl)-2-ethynylthiazole-4-carboxamide C51;
N-(3-chlorobenzyl)-2-ethynyl-5-methylthiazole-4-carboxamide C52;
2-ethynyl-N-(4-(thiazol-4-yl)phenethyl)thiazole-4-carboxamide C53;
2-ethynyl-N-(4-(1-methyl-1H-pyrazol-3-yl)benzyl)thiazole-4-carboxamide C54;
2-ethynyl-N-(4-(thiazol-4-yl)benzyl)thiazole-4-carboxamide C55;
N-(3-chlorobenzyl)-2-ethynyl-5-phenylthiazole-4-carboxamide C56;
2-ethynyl-N-(4-(thiazol-5-yl)phenethyl)thiazole-4-carboxamide C57;
N-(3-(1H-pyrazol-4-yl)benzyl)-2-ethynylthiazole-4-carboxamide C58;
2-ethynyl-N-(3-(1-methyl-1H-pyrazol-4-yl)benzyl)thiazole-4-carboxamide C59;
N-(3-(1H-pyrazol-3-yl)benzyl)-2-ethynylthiazole-4-carboxamide C60;
2-ethynyl-N-(4-(thiazol-5-yl)benzyl)thiazole-4-carboxamide C61;
N-(2,3-dihydro-11H-inden-2-yl)-2-ethynylthiazole-4-carboxamide C62;
N-(2-cyanobenzyl)-2-ethynylthiazole-4-carboxamide C63;
2-ethynyl-N-(naphthalen-2-ylmethyl)thiazole-4-carboxamide C64;
2-ethynyl-N-(3-(pyridin-3-yl)benzyl)thiazole-4-carboxamide C65:
N-(benzo[d]thiazol-6-ylmethyl)-2-ethynylthiazole-4-carboxamide C66;
(R)—N-(1-(3-chlorophenyl)ethyl)-2-ethynylthiazole-4-carboxamide C67;
N-(1-(3-chlorophenyl)cyclopropyl)-2-ethynylthiazole-4-carboxamide C68:
(S)—N-(1-(3-chlorophenyl)ethyl)-2-ethynylthiazole-4-carboxamide C69;
2-ethynyl-N-(2-(hydroxymethyl)benzyl)thiazole-4-carboxamide C70;
(S)—N-(2,3-dihydro-1H-inden-1-yl)-2-ethynylthiazole-4-carboxamide C71;
(R)—N-(2,3-dihydro-1H-inden-1-yl)-2-ethynylthiazole-4-carboxamide C72;
methyl 3-(4-((2-ethynylthiazole-4-carboxamido)methyl)phenyl)propanoate C73;
methyl 2-((2-ethynylthiazole-4-carboxamido)methyl)benzoate C74;
2-ethynyl-N-(4-(1-methyl-1H-pyrazol-4-yl)phenethyl)thiazole-4-carboxamide C75;
N-(4-(1H-pyrazol-4-yl)phenethyl)-2-ethynylthiazole-4-carboxamide C76;
N-(2-ethynylthiazol-4-yl)-2-phenylacetamide C77;
2-ethynyl-N-(1-phenylpiperidin-4-yl)thiazole-4-carboxamide C78;
2-ethynyl-N-(4-(pyridin-3-yl)phenethyl)thiazole-4-carboxamide C79;
N-(4-bromophenethyl)-2-ethynylthiazole-4-carboxamide C80;
2-ethynyl-N-(3-(methylsulfonamido)phenethyl)thiazole-4-carboxamide C81;
N-(3-acetamidophenethyl)-2-ethynylthiazole-4-carboxamide C82;
2-ethynyl-N-(4-(1-methyl-1H-pyrazol-4-yl)benzyl)thiazole-4-carboxamide C83;
methyl 4-(2-(2-ethynylthiazole-4-carboxamido)ethyl)benzoate C84;
N-(3-aminophenethyl)-2-ethynylthiazole-4-carboxamide C85:
N-((2,3-dihydrobenzo[b][1,4]dioxin-6-yl)methyl)-2-ethynylthiazole-4-carboxamide C86;
2-ethynyl-N-(3-nitrophenethyl)thiazole-4-carboxamide C87;
N-(4-acetamidophenethyl)-2-ethynylthiazole-4-carboxamide C88;
N-(4-acetamidobenzyl)-2-ethynylthiazole-4-carboxamide C89;
N-benzyl-2-cyanothiazole-4-carboxamide C90;
N-(4-(1H-pyrazol-3-yl)benzyl)-2-ethynylthiazole-4-carboxamide C91;
N-(4-(1H-pyrazol-4-yl)benzyl)-2-ethynylthiazole-4-carboxamide C92:
2-ethynyl-N-(4-(pyridin-3-yl)benzyl)thiazole-4-carboxamide C93;
N-(3-acetamidobenzyl)-2-ethynylthiazole-4-carboxamide C94;
N-(3-aminobenzyl)-2-ethynylthiazole-4-carboxamide C95;
2-ethynyl-N-(quinolin-6-ylmethyl)thiazole-4-carboxamide C96;
methyl 3-(2-(2-ethynylthiazole-4-carboxamido)ethyl)benzoate C97;
N-benzyl-2-ethynyl-1-methyl-1H-imidazole-4-carboxamide C98;
2-ethynyl-N-(4-(methylsulfonamido)phenethyl)thiazole-4-carboxamide C99:
2-ethynyl-N-methyl-N-(4-nitrophenethyl)thiazole-4-carboxamide C100:
2-ethynyl-N-(4-(methylsulfonyl)phenethyl)thiazole-4-carboxamide C101;
N-(4-cyanophenethyl)-2-ethynylthiazole-4-carboxamide C102:
2-ethynyl-N-(4-nitrobenzyl)thiazole-4-carboxamide C103;
N-(4-cyanobenzyl)-2-ethynylthiazole-4-carboxamide C104:
2-ethynyl-N-(3-(methylsulfonyl)phenethyl)thiazole-4-carboxamide C105;
N-(2-chlorophenethyl)-2-ethynylthiazole-4-carboxamide C106;
2-ethynyl-N-(3-nitrobenzyl)thiazole-4-carboxamide C107;
2-ethynyl-N-(4-(methylsulfonyl)benzyl)thiazole-4-carboxamide C108;
N-((3-chloropyridin-2-yl)methyl)-2-ethynylthiazole-4-carboxamide C109;
N-(3-chlorophenethyl)-2-ethynylthiazole-4-carboxamide C110;
N-benzyl-2-ethynyl-1H-imidazole-4-carboxamide C111:
N-([1,1′-biphenyl]-2-ylmethyl)-2-ethynylthiazole-4-carboxamide C112;
2-ethynyl-N-(naphthalen-1-ylmethyl)thiazole-4-carboxamide C113:
N-([1,1′-biphenyl]-4-ylmethyl)-2-ethynylthiazole-4-carboxamide C114;
N-([1,1′-biphenyl]-3-ylmethyl)-2-ethynylthiazole-4-carboxamide C115:
ethyl 3-(N-benzyl-2-ethynylthiazole-4-carboxamido)propanoate C116;
methyl 4-((2-ethynylthiazole-4-carboxamido)methyl)benzoate C117;
N-(4-aminobenzyl)-2-ethynylthiazole-4-carboxamide C118;
2-ethynyl-N-((1-methyl-1H-pyrazol-3-yl)methyl)thiazole-4-carboxamide C119;
2-ethynyl-N-((1-methyl-1H-pyrazol-4-yl)methyl)thiazole-4-carboxamide C120;
2-ethynyl-N-(4-hydroxybenzyl)thiazole-4-carboxamide C121:
2-ethynyl-N-(4-hydroxy-3-methoxyphenethyl)thiazole-4-carboxamide C122;
N-(2-ethynylthiazol-4-yl)-3-phenylpropanamide C123;
methyl 3-((2-ethynylthiazole-4-carboxamido)methyl)benzoate C124:
N-(3-chlorobenzyl)-2-ethynylthiazole-4-carboxamide C125:
2-ethynyl-N-(pyridin-2-ylmethyl)thiazole-4-carboxamide C126:
N-(3-cyanobenzyl)-2-ethynylthiazole-4-carboxamide C127;
N-(4-aminophenethyl)-2-ethynylthiazole-4-carboxamide C128:
2-ethynyl-N-(4-nitrophenethyl)thiazole-4-carboxamide C129:
2-ethynyl-N-(1H-indazol-4-yl)thiazole-4-carboxamide C130;
ethyl N-benzyl-N-(2-ethynylthiazole-4-carbonyl)glycinate C133;
N-benzyl-2-ethynyl-1-methyl-11H-imidazole-5-carboxamide C134:
N-benzyl-2-ethynyl-N-(2-hydroxyethyl)thiazole-4-carboxamide C135:
2-ethynyl-N-(1H-indazol-7-yl)thiazole-4-carboxamide C136:
(S)-2-ethynyl-N-(2-hydroxy-2-phenylethyl)thiazole-4-carboxamide C137;
N-(2-acetamidobenzyl)-2-ethynylthiazole-4-carboxamide C138:
N-benzyl-2-ethynyl-N-methylthiazole-4-carboxamide C139;
2-ethynyl-N-phenethylthiazole-4-carboxamide C140;
N-((1H-indol-4-yl)methyl)-2-ethynylthiazole-4-carboxamide C141:
N-(2-aminobenzyl)-2-ethynylthiazole-4-carboxamide C142:
N-benzyl-2-ethynylthiazole-5-carboxamide C143;
N-benzyl-2-ethynyloxazole-4-carboxamide C144;
N-(2-chlorobenzyl)-2-ethynylthiazole-4-carboxamide C145:
2-ethynyl-N-(2-methoxybenzyl)thiazole-4-carboxamide C146;
N-benzyl-4-ethynylthiazole-2-carboxamide C147;
N-benzyl-2-ethynylpyrimidine-4-carboxamide C148:
N-benzyl-6-ethynylpicolinamide C149;
N-benzyl-2-ethynylthiazole-4-carboxamide C150; or
N-benzyl-2-ethynylisonicotinamide C151;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In yet another embodiment, provided herein is a compound of:
(4-(4-(1,5-dimethyl-1H-indazol-4-yl)phenyl)piperazin-1-yl)(2-ethynylthiazol-4-yl)methanone D1:
methyl 4-(4-(4-(2-ethynylthiazole-4-carbonyl)piperazin-1-yl)phenyl)-1-methyl-1H-indazole-6-carboxylate D2;
7-(4-(4-(2-ethynylthiazole-4-carbonyl)piperazin-1-yl)phenyl)-N-methylbenzo[d]thiazole-5-carboxamide D3;
methyl 7-(4-(4-(2-ethynylthiazole-4-carbonyl)piperazin-1-yl)phenyl)benzo[d]-thiazole-5-carboxylate D4;
7-(4-(4-(2-ethynylthiazole-4-carbonyl)piperazin-1-yl)phenyl)benzo[d]thiazole-5-carboxamide D5;
7-(4-(4-(2-ethynylthiazole-4-carbonyl)piperazin-1-yl)phenyl)-N,N-dimethylbenzo[d]-thiazole-5-carboxamide D6;
(4-(4-(benzo[d]thiazol-7-yl)phenyl)piperazin-1-yl)(2-ethynylthiazol-5-yl)methanone D7;
(4-(4-(benzo[d]thiazol-4-yl)phenyl)piperazin-1-yl)(2-ethynylthiazol-5-yl)methanone D8;
(4-(4-(benzo[d]thiazol-7-yl)-2-chlorophenyl)piperazin-1-yl)(2-ethynylthiazol-4-yl)methanone D9;
(4-(5-(benzo[d]thiazol-7-yl)pyridin-2-yl)piperazin-1-yl)(2-ethynylthiazol-4-yl)methanone D10;
(4-(4-(benzo[d]thiazol-7-yl)phenyl)-4-fluoropiperidin-1-yl)(2-ethynylthiazol-4-yl)methanone D11;
5-(benzo[d]thiazol-7-yl)-2-(4-(2-ethynylthiazole-4-carbonyl)piperazin-1-yl)benzonitrile D12;
(3-(4-(benzo[d]thiazol-7-yl)phenyl)azetidin-1-yl)(2-ethynylthiazol-4-yl)methanone D13:
(4-(4-(benzo[d]thiazol-7-yl)phenyl)-4-hydroxypiperidin-1-yl)(2-ethynylthiazol-4-yl)methanone D14;
(4-(3-(benzo[d]thiazol-7-yl)phenyl)piperidin-1-yl)(2-ethynylthiazol-4-yl)methanone D15:
(4-(3-(benzo[d]thiazol-7-yl)phenyl)piperazin-1-yl)(2-ethynylthiazol-4-yl)methanone D16;
(4-(4-(benzo[d]thiazol-7-yl)phenyl)piperazin-1-yl)(2-ethynylthiazol-4-yl)methanone D17;
(4-(4-(benzo[d]thiazol-7-yl)phenyl)piperidin-1-yl)(2-ethynylthiazol-4-yl)methanone D18;
(2-ethynylthiazol-4-yl)(4-(4-(2-methyl-2H-indazol-4-yl)phenyl)piperidin-1-yl)methanone D19;
(2-ethynylthiazol-4-yl)(4-(4-(1-methyl-1H-indazol-4-yl)phenyl)piperidin-1-yl)methanone D20;
(4-(4-(1H-indazol-4-yl)phenyl)piperidin-1-yl)(2-ethynylthiazol-4-yl)methanone D21;
(2-ethynylthiazol-4-yl)(4-(4-(1-methyl-1H-indazol-4-yl)phenyl)piperazin-1-yl)methanone D22;
(2-ethynylthiazol-4-yl)(4-(4-(2-methyl-2H-indazol-4-yl)phenyl)piperazin-1-yl)methanone D23;
4-(1-(2-ethynylthiazole-4-carbonyl)piperidin-4-yl)benzonitrile D24;
(4-(4-(1H-indazol-4-yl)phenyl)piperazin-1-yl)(2-ethynylthiazol-4-yl)methanone D25;
4-(4-(2-ethynylthiazole-4-carbonyl)piperazin-1-yl)benzonitrile D26;
(2-ethynylthiazol-4-yl)(4-(4-nitrophenyl)piperazin-1-yl)methanone D27;
(2-ethynylthiazol-4-yl)(4-phenylpiperazin-1-yl)methanone D28;
(2-ethynylthiazol-4-yl)(4-phenylpiperidin-1-yl)methanone D29;
(3,4-dihydroisoquinolin-2(1H)-yl)(2-ethynylthiazol-4-yl)methanone D30;
(2-ethynylthiazol-4-yl)(4-hydroxypiperidin-1-yl)methanone D31;
(2-ethynylthiazol-4-yl)(2-(2-hydroxyethyl)piperidin-1-yl)methanone D32:
(2-ethynylthiazol-4-yl)(3-(hydroxymethyl)piperidin-1-yl)methanone D33;
(2-ethynylthiazol-4-yl)(3-hydroxypiperidin-1-yl)methanone D34;
methyl 1-(2-ethynylthiazole-4-carbonyl)piperidine-4-carboxylate D35;
methyl 1-(2-ethynylthiazole-4-carbonyl)piperidine-3-carboxylate D36; or
methyl 1-(2-ethynylthiazole-4-carbonyl)piperidine-2-carboxylate D37;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In still another embodiment, provided herein is:
3-(4-(benzo[d]thiazol-7-yl)phenyl)-N-(2-ethynylthiazol-4-yl)propanamide E1;
(S)-2-amino-N-(2-ethynylthiazol-4-yl)-3-phenylpropanamide E2;
(R)-2-amino-N-(2-ethynylthiazol-4-yl)-3-phenylpropanamide E3;
N-(2-ethynylthiazol-4-yl)-3-(4-(1-methyl-1H-indazol-4-yl)phenyl)propanamide E4:
(R)-2-amino-3-(2-cyanophenyl)-N-(2-ethynylthiazol-4-yl)propenamide E5;
2-ethynyl-N-(3-(thiazol-5-yl)phenethyl)thiazole-4-carboxamide E6:
2-ethynyl-N-(3-(thiazol-4-yl)phenethyl)thiazole-4-carboxamide E7;
(S)-2-amino-3-(2-cyanophenyl)-N-(2-ethynylthiazol-4-yl)propenamide E8;
3-(benzo[d][1,3]dioxol-5-yl)-N-(2-ethynylthiazol-4-yl)propanamide E9:
N-(2-ethynylthiazol-4-yl)quinoline-2-carboxamide E10;
N-(2-ethynylthiazol-4-yl)cinnamamide E11; or
N-(2-ethynylthiazol-4-yl)benzamide E12;
   or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, a compound provided herein is deuterium-enriched. In certain embodiments, a compound provided herein is carbon-13 enriched. In certain embodiments, a compound provided herein is carbon-14 enriched. In certain embodiments, a compound provided herein contains one or more less prevalent isotopes for other elements, including, but not limited to, ¹⁵N for nitrogen; ¹⁷O or ¹⁸O for oxygen, and ³⁴S, ³⁵S, or ³⁶S for sulfur.

In certain embodiments, a compound provided herein has an isotopic enrichment factor of no less than about 5, no less than about 10, no less than about 20, no less than about 50, no less than about 100, no less than about 200, no less than about 500, no less than about 1,000, no less than about 2,000, no less than about 5,000, or no less than about 10,000. In any events, however, an isotopic enrichment factor for a specified isotope is no greater than the maximum isotopic enrichment factor for the specified isotope, which is the isotopic enrichment factor when a compound at a given position is 100% enriched with the specified isotope. Thus, the maximum isotopic enrichment factor is different for different isotopes. The maximum isotopic enrichment factor is 6,410 for deuterium and 90 for carbon-13.

In certain embodiments, a compound provided herein has a deuterium enrichment factor of no less than about 64 (about 1% deuterium enrichment), no less than about 130 (about 2% deuterium enrichment), no less than about 320 (about 5% deuterium enrichment), no less than about 640 (about 10% deuterium enrichment), no less than about 1,300 (about 20% deuterium enrichment), no less than about 3,200 (about 50% deuterium enrichment), no less than about 4,800 (about 75% deuterium enrichment), no less than about 5,130 (about 80% deuterium enrichment), no less than about 5,450 (about 85% deuterium enrichment), no less than about 5,770 (about 90% deuterium enrichment), no less than about 6,090 (about 95% deuterium enrichment), no less than about 6,220 (about 97% deuterium enrichment), no less than about 6.280 (about 98% deuterium enrichment), no less than about 6,350 (about 99% deuterium enrichment), or no less than about 6,380 (about 99.5% deuterium enrichment). The deuterium enrichment can be determined using conventional analytical methods known to one of ordinary skill in the art, including mass spectrometry and nuclear magnetic resonance spectroscopy. In certain embodiments, at least one of the atoms of a compound provided herein, as specified as deuterium-enriched, has deuterium enrichment of no less than about 1%, no less than about 2%, no less than about 5%, no less than about 10%, no less than about 20%, no less than about 50%, no less than about 70%, no less than about 80%, no less than about 90%, or no less than about 98%.

In certain embodiments, a compound provided herein is isolated or purified. In certain embodiments, a compound provided herein has a purity of at least about 90%, at least about 95%, at least about 98%, at least about 99%, or at least about 99.5% by weight.

The compounds provided herein are intended to encompass all possible stereoisomers, unless a particular stereochemistry is specified. Where a compound provided herein contains an alkenyl group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contains an aromatic moiety. It follows that a single compound may exhibit more than one type of isomerism.

The compound provided herein can be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of ordinary skill in the art will recognize that administration of a compound in its (R) form is equivalent, for the compound that undergoes epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.: John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid. L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (i)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions

In one embodiment, provided herein is a pharmaceutical composition, comprising a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; and a pharmaceutically acceptable excipient.

The pharmaceutical composition provided herein can be formulated in various dosage forms, including, but not limited to, dosage forms for oral, parenteral, and topical administration. The pharmaceutical composition can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art. See, e.g., Remington: The Science and Practice of Pharmacy, supra; Modified-Release Drug Delivery Technology, 2nd ed.; Rathbone et al., Eds.; Drugs and the Pharmaceutical Sciences 184; CRC Press: Boca Raton, F L, 2008.

In one embodiment, the pharmaceutical composition provided herein is formulated in a dosage form for oral administration. In another embodiment, the pharmaceutical composition provided herein is formulated in a dosage form for parenteral administration. In yet another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for topical administration.

The pharmaceutical composition provided herein can be provided in a unit-dosage form or multiple-dosage form. A unit-dosage form, as used herein, refers to physically discrete a unit suitable for administration to a subject, and packaged individually as is known in the art. Each unit-dose contains a predetermined quantity of an active ingredient(s) (e.g., a compound provided herein) sufficient to produce the desired therapeutic effect, in association with the required pharmaceutical excipient(s). Examples of a unit-dosage form include, but are not limited to, an ampoule, syringe, and individually packaged tablet and capsule. A unit-dosage form may be administered in fractions or multiples thereof. A multiple-dosage form is a plurality of identical unit-dosage forms packaged in a single container to be administered in a segregated unit-dosage form. Examples of a multiple-dosage form include, are not limited to, a vial, bottle of tablets or capsules, or bottle of pints or gallons.

The pharmaceutical composition provided herein can be administered at once or multiple times at intervals of time. It is understood that the precise dosage and duration of treatment may vary with the age, weight, and condition of the subject being treated, and may be determined empirically using known testing protocols or by extrapolation from in vivo or in vitro test or diagnostic data. It is further understood that for any particular individual, specific dosage regimens should be adjusted over time according to the subject's need and the professional judgment of the person administering or supervising the administration of the pharmaceutical composition.

### A. Oral Administration

The pharmaceutical composition provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical composition can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500®); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), VEEGUM®, larch arabinogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); and microcrystalline celluloses, such as AVICEL® PH-101, AVICEL® PH-103, AVICEL® PH-105, and AVICEL® RC-581. Suitable fillers include, but are not limited to, talc, calcium carbonate, microcrystalline cellulose, powdered cellulose, dextrates, kaolin, mannitol, silicic acid, sorbitol, starch, and pre-gelatinized starch. The amount of a binder or filler in the pharmaceutical composition provided herein varies upon the type of formulation, and is readily discernible to those of ordinary skill in the art. The binder or filler may be present from about 50 to about 99% by weight in the pharmaceutical composition provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical composition provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and VEEGUM® HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and algins. The amount of a disintegrant in the pharmaceutical composition provided herein varies upon the type of formulation, and is readily discernible to those of ordinary skill in the art. The pharmaceutical composition provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, such as peanut oil, cottonseed oil, sunflower oil, sesame oil, olive oil, corn oil, and soybean oil; zinc stearate; ethyl oleate; ethyl laureate; agar; starch; lycopodium; and silica or silica gels, such as AEROSIL® 200 and CAB-O-SIL®. The amount of a lubricant in the pharmaceutical composition provided herein varies upon the type of formulation, and is readily discernible to those of ordinary skill in the art. The pharmaceutical compositions provided herein may contain about 0.1 to about 5% by weight of a lubricant.

Suitable glidants include, but are not limited to, colloidal silicon dioxide, CAB-O-SIL®, and asbestos-free talc. Suitable coloring agents include, but are not limited to, any of the approved, certified, water soluble FD&C dyes, and water insoluble FD&C dyes suspended on alumina hydrate, and color lakes. A color lake is a combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, VEEGUM®, acacia, sodium carboxymethylcellulose, hydroxypropyl methylcellulose, and polyvinylpyrrolidone. Suitable preservatives include, but are not limited to, glycerin, methyl and propylparaben, benzoic add, and sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical composition provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient(s) from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from an active ingredient(s) in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical composition provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient(s). The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient(s).

The pharmaceutical composition provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing an active ingredient(s), and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These dosage forms can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical composition provided herein for oral administration can be also provided in the forms of liposomes, micelles, microspheres, or nanosystems. Micellar dosage forms can be prepared as described in U.S. Pat. No. 6,350,458.

The pharmaceutical composition provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the dosage forms described herein.

The pharmaceutical composition provided herein for oral administration can be formulated as immediate or modified release dosage forms, including delayed-, sustained, pulsed-, controlled, targeted-, and programmed-release forms.

### B. Parenteral Administration

The pharmaceutical composition provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical composition provided herein for parenteral administration can be formulated in any dosage forms that are suitable for parenteral administration, including, but not limited to, solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science. See, e.g., Remington: The Science and Practice of Pharmacy, supra.

The pharmaceutical composition provided herein for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringer's injection, isotonic dextrose injection, sterile water injection, dextrose and lactated Ringer's injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants include those described herein, such as bisulfite and sodium metabisulfite. Suitable local anesthetics include, but are not limited to, procaine hydrochloride. Suitable suspending and dispersing agents include those described herein, such as sodium carboxymethylcellulose, hydroxypropyl methylcellulose, and polyvinylpyrrolidone. Suitable emulsifying agents include those described herein, such as polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®).

When the pharmaceutical composition provided herein is formulated for multiple dosage administration, multiple dosage parenteral formulations must contain an antimicrobial agent at bacteriostatic or fungistatic concentrations. All parenteral formulations must be sterile, as known and practiced in the art.

In one embodiment, the pharmaceutical composition for parenteral administration is provided as a ready-to-use sterile solution. In another embodiment, the pharmaceutical composition is provided as a sterile dry soluble product, including a lyophilized powder and hypodermic tablet, to be reconstituted with a vehicle prior to use. In yet another embodiment, the pharmaceutical composition is provided as a ready-to-use sterile suspension. In yet another embodiment, the pharmaceutical composition is provided as a sterile dry insoluble product to be reconstituted with a vehicle prior to use. In still another embodiment, the pharmaceutical composition is provided as a ready-to-use sterile emulsion.

The pharmaceutical composition provided herein for parenteral administration can be formulated as immediate or modified release dosage forms, including delayed-, sustained, pulsed-, controlled, targeted-, and programmed-release forms.

The pharmaceutical composition provided herein for parenteral administration can be formulated as a suspension, solid, semi-solid, or thixotropic liquid, for administration as an implanted depot. In one embodiment, the pharmaceutical composition provided herein are dispersed in a solid inner matrix, which is surrounded by an outer polymeric membrane that is insoluble in body fluids but allows the active ingredient(s) in the pharmaceutical composition to diffuse through.

Suitable inner matrixes include, but are not limited to, polymethylmethacrylate, polybutylmethacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers (such as hydrogels of esters of acrylic and methacrylic acid), collagen, cross-linked polyvinyl alcohol, and cross-linked partially hydrolyzed polyvinyl acetate.

Suitable outer polymeric membranes include, but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical composition provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical composition provided herein can be formulated in any dosage forms that are suitable for topical administration for local or systemic effect, including, but not limited to, emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulations of the pharmaceutical composition provided herein can also comprise liposomes, micelles, microspheres, and nanosystems.

Pharmaceutically acceptable carriers and excipients suitable for use in the topical formulations include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical composition can also be administered topically by electroporation, iontophoresis, phonophoresis, sonophoresis, or microneedle or needle-free injection, such as POWDERJECT™ and BIOJECT™.

The pharmaceutical composition provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid. See, e.g., Remington: The Science and Practice of Pharmacy, supra. These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical composition provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with an active ingredient(s); and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical composition provided herein can be administered ophthalmically in the forms of solutions, suspensions, ointments, emulsions, gel-forming solutions, powders for solutions, gels, ocular inserts, and implants.

The pharmaceutical composition provided herein can be administered intranasally or by inhalation to the respiratory tract. The pharmaceutical composition can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical composition can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of an active ingredient(s); a propellant as solvent; and/or a surfactant, such as sorbitan trioleate, oleic acid, or an oligolactic acid.

The pharmaceutical composition provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical composition provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical composition provided herein for inhaled/intranasal administration can further comprise a suitable flavor, such as menthol and levomenthol; and/or sweeteners, such as saccharin and saccharin sodium.

The pharmaceutical composition provided herein for topical administration can be formulated to be immediate release or modified release, including delayed-, sustained-, pulsed-, controlled-, targeted, and programmed release.

### D. Modified Release

The pharmaceutical composition provided herein can be formulated as a modified release dosage form. As used herein, the term “modified release” refers to a dosage form in which the rate or place of release of an active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical composition in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

### 1. Matrix Controlled Release Devices

The pharmaceutical composition provided herein in a modified release dosage form can be fabricated using a matrix controlled release device known to those skilled in the art. See, e.g., Takada et al. in Encyclopedia of Controlled Drug Delivery, Mathiowitz Ed.; Wiley, 1999; Vol. 2.

In certain embodiments, the pharmaceutical composition provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical composition provided herein is formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical composition provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical composition provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical composition in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, e.g., Remington: The Science and Practice of Pharmacy, supra; Santus and Baker, J. Controlled Release, 1995, 35, 1-21; Verma et al., Drug Dev. Ind. Pharm., 2000, 26, 695-708; Verma et al., J. Controlled Release, 2002, 79, 7-27.

In certain embodiments, the pharmaceutical composition provided herein is formulated as an AMT controlled-release dosage form, which comprises an asymmetric osmotic membrane that coats a core comprising the active ingredient(s) and other pharmaceutically acceptable excipients or carriers. See, e.g., U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical composition provided herein is formulated as an ESC controlled-release dosage form, which comprises an osmotic membrane that coats a core comprising the active ingredient(s), a hydroxyethyl cellulose, and other pharmaceutically acceptable excipients or carriers.

### 3. Multiparticulate Controlled Release Devices

The pharmaceutical composition provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, e.g., Multiparticulate Oral Drug Delivery; Ghebre-Sellassie Eds.; Drugs and the Pharmaceutical Sciences 65; CRC Press: 1994; and Pharmaceutical Pelletization Technology: Ghebre-Sellassie Eds.; Drugs and the Pharmaceutical Sciences 37; CRC Press: 1989.

Other excipients or carriers as described herein can be blended with the pharmaceutical composition to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical composition provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein is a method of treating, ameliorating, or preventing one or more symptoms of a proliferative disease in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is a method of treating, ameliorating, or preventing one or more symptoms of a disorder, disease, or condition mediated by a glutathione peroxidase 4 (GPX4) in a subject, comprising administering to the subject in need thereof a therapeutically effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof. In one embodiment, the disorder, disease, or condition mediated by GPX4 is a proliferative disease.

In certain embodiments, the proliferative disease is cancer. In certain embodiments, the cancer is liver cancer. In certain embodiments, the cancer is metastatic. In certain embodiments, the cancer is refractory. In certain embodiments, the cancer is relapsed. In certain embodiments, the cancer is drug-resistant. In certain embodiments, the cancer is multidrug-resistant.

In certain embodiments, the subject is a mammal. In certain embodiments, the subject is a human.

In certain embodiments, the therapeutically effective amount of a compound provided herein is ranging from about 0.1 to about 100 mg/kg/day, from about 0.1 to about 50 mg/kg/day, from about 0.1 to about 60 mg/kg/day, from about 0.1 to about 50 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount is ranging from about 0.1 to about 100 mg/kg/day. In another embodiment, the therapeutically effective amount is ranging from about 0.1 to about 50 mg/kg/day. In yet another embodiment, the therapeutically effective amount is ranging from about 0.1 to about 60 mg/kg/day. In yet another embodiment, the therapeutically effective amount is ranging from about 0.1 to about 50 mg/kg/day. In yet another embodiment, the therapeutically effective amount is ranging from about 0.1 to about 25 mg/kg/day. In yet another embodiment, the therapeutically effective amount is ranging from about 0.1 to about 20 mg/kg/day. In yet another embodiment, the therapeutically effective amount is ranging from about 0.1 to about 15 mg/kg/day. In yet another embodiment, the therapeutically effective amount is ranging from about 0.1 to about 10 mg/kg/day. In still another embodiment, the therapeutically effective amount is ranging from about 0.1 to about 5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both. For example, a dose of 1 mg/m²/day for a 65 kg human is approximately equal to 58 mg/kg/day.

Depending on the disease to be treated and the subject's condition, a compound provided herein may be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A compound provided herein may be formulated in suitable dosage unit with a pharmaceutically acceptable excipient, carrier, adjuvant, or vehicle, appropriate for each route of administration.

In one embodiment, a compound provided herein is administered orally. In another embodiment, a compound provided herein is administered parenterally. In yet another embodiment, a compound provided herein is administered intravenously. In yet another embodiment, a compound provided herein is administered intramuscularly. In yet another embodiment, a compound provided herein is administered subcutaneously. In still another embodiment, a compound provided herein is administered topically.

A compound provided herein can be delivered as a single dose such as, e.g., a single bolus injection, or oral tablets or pills; or over time such as, e.g., continuous infusion over time or divided bolus doses over time. A compound provided herein can be administered repetitively if necessary, for example, until the subject experiences stable disease or regression, or until the patient experiences disease progression or unacceptable toxicity. Stable disease or lack thereof is determined by methods known in the art such as evaluation of subject's symptoms, physical examination, visualization of the cancer that has been imaged using X-ray, CAT, PET, or MRI scan and other commonly accepted evaluation modalities.

A compound provided herein can be administered once daily (QD) or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of a compound provided herein is administration for one to six days per week, administration in cycles (e.g., daily administration for two to eight consecutive weeks, then a rest period with no administration for up to one week), or administration on alternate days.

In certain embodiments, a compound provided herein is cyclically administered to a subject. Cycling therapy involves the administration of an active agent for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

A compound provided herein can also be combined or used in combination with other therapeutic agents useful in the treatment and/or prevention of a condition, disorder, or disease described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 50 minutes, 65 minutes, 1 hour, 2 hours, 6 hours, 6 hours, 12 hours, 26 hours, 68 hours, 72 hours, 96 hours, 1 week, 2 weeks, 5 weeks, 6 weeks, 8 weeks, or 12 weeks before), concomitantly with, or subsequent to (e.g., 5 minutes, 15 minutes, 50 minutes, 65 minutes, 1 hour, 2 hours, 6 hours, 12 hours, 26 hours, 68 hours, 72 hours, 96 hours, 1 week, 2 weeks, 5 weeks, 6 weeks, 8 weeks, or 12 weeks after) the administration of a second therapy (e.g., a prophylactic or therapeutic agent) to the subject. Triple therapy is also contemplated herein.

The route of administration of a compound provided herein is independent of the route of administration of a second therapy. In one embodiment, a compound provided herein is administered orally. In another embodiment, a compound provided herein is administered intravenously. Thus, in accordance with these embodiments, a compound provided herein is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraocularly, via local delivery by catheter or stent, subcutaneously, intraadiposally, intraarticularly, intrathecally, or in a slow release dosage form. In one embodiment, a compound provided herein and a second therapy are administered by the same mode of administration, orally or by IV. In another embodiment, a compound provided herein is administered by one mode of administration, e.g., by IV, whereas the second agent (an anticancer agent) is administered by another mode of administration, e.g., orally.

In one embodiment, provided herein is a method of inhibiting the growth of a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In another embodiment, provided herein is a method of inducing ferroptosis in a cell, comprising contacting the cell with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the cell is a cancerous cell. In certain embodiments, the cell is a cell of hepatic cancer.

In one embodiment, provided herein is a method of irreversibly inhibiting the activity of a protein, comprising contacting the protein with a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In one embodiment, the protein is an enzyme. In another embodiment, the protein is a kinase, GTPase, or protease. In yet another embodiment, the protein is a protein kinase. In yet another embodiment, the protein is a tyrosine kinase. In yet another embodiment, the protein is a receptor tyrosine kinase. In yet another embodiment, the protein is an epidermal growth factor receptor. In yet another embodiment, the protein is an EGFR (ErbB-1), HER2 (ErbB-2), HER3 (ErbB-3), or HER4 (Erb-4). In yet another embodiment, the protein is a Bruton's tyrosine kinase (BTK). In yet another embodiment, the protein is a GTPase. In yet another embodiment, the protein is a Ras GTPase. In yet another embodiment, the protein is a KRas GTPase. In yet another embodiment, the protein is a protease. In still another embodiment, the protein is a GPX4.

In another embodiment, provided herein is a method of inhibiting the activity of a GPX4, comprising contacting the GPX4 with an effective amount of a compound of Formula (I), or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

A compound provided herein can also be provided as an article of manufacture using packaging materials well known to those of skill in the art. See, e.g., U.S. Pat. Nos. 5,525,907; 5,052,558; and 5,055,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein is a kit which, when used by a medical practitioner, can simplify the administration of an appropriate amount of a compound provided herein as an active ingredient to a subject. In certain embodiments, the kit provided herein includes a container and a dosage form of a compound provided herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, water for injection USP, sodium chloride injection, Ringer's injection, dextrose injection, dextrose and sodium chloride injection, and lactated Ringer's injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry. Specifically, but without limitation, the following abbreviations may be used in the examples and throughout the specification: g (grams); mg (milligrams); mL (milliliters); μL (microliters); mM (millimolar); M (micromolar); mmol (millimoles); h (hour or hours); min (minutes); EtOH (ethanol); MeOH (methanol); EtOAc (ethyl acetate); prep-TLC (preparative thin-layer chromatography); LCMS (liquid chromatography-mass spectrometry); and NMR (nuclear magnetic resonance).

For all of the following examples, standard work-up and purification methods known to those skilled in the art can be utilized. Unless otherwise indicated, all temperatures are expressed in ° C. (degrees Centigrade). All reactions are conducted at room temperature unless otherwise specified. Synthetic methodologies illustrated herein are intended to exemplify the applicable chemistry through the use of specific examples and are not indicative of the scope of the disclosure.

### Example 1

### Preparation of 1-(4-(benzo[d]thiazol-7-yl)benzyl)-3-(2-ethynylthiazol-4-yl)urea A6

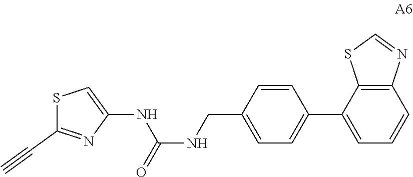 [SMILES: C#Cc1nc(NC(=O)NCc2ccc(-c3cccc4ncsc34)cc2)cs1]

Compound A6 was synthesized as shown in Schemes 1 and 2.

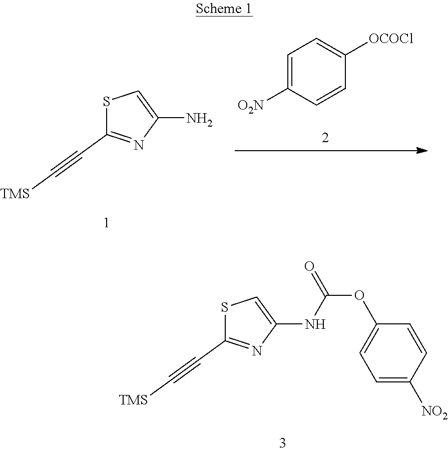

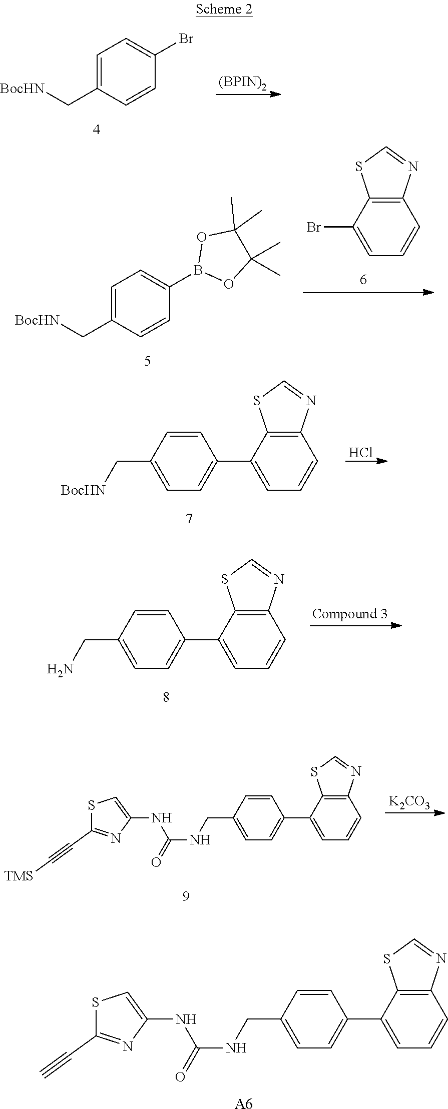

4-Nitrophenyl(2-((trimethylsilyl)ethynyl)thiazol-4-yl)carbamate 3. A mixture of compound 1 (100 mg, 0.51 mmol), compound 2 (128 mg, 0.64 mmol), and pyridine (0.5 mL) in DCM (4 mL) was stirred at room temperature for 3 h. The reaction solution was then diluted with HCl (15 mL, 1 M in H₂O) and extracted with DCM (15 mL×3). The organic layers were combined, washed with brine (20 mL×2), dried over anhydrous Na₂SO₄, concentrated, and purified by flash chromatography (PE/EA: 100/0 to 50/50) to yield compound 3 (60.4 mg) in 32% yield. LCMS (ESI) m/z: 362.0 [M+H⁺].

Tert-butyl (4-(4,4,5,5-tetramethyl-1,3,2-dioxaborolan-2-yl)benzyl)carbamate 5. A mixture of compound 4 (950 mg, 3.32 mmol), Pd(dppf)Cl₂ (121.5 mg, 0.17 mmol), (BPIN)₂ (1010 mg, 3.93 mmol), and KOAc (976 mg, 9.66 mmol) in dry dioxane (20 mL) was stirred at 100° C. under nitrogen for 3 h. The reaction mixture was then concentrated and purified by flash chromatography (PE/EA: 100/0 to 90/10) to yield compound 5 (918.5 mg) in 83% yield. LCMS (ESI) m/z: 278.1 [M+H⁺-56].

Tert-butyl (4-(benzo[d]thiazol-7-yl)benzyl)carbamate 7. A mixture of compound 5 (200 mg, 0.60 mmol), compound 6 (141 mg, 0.66 mmol), Pd(dppf)Cl₂ (40 mg, 0.05 mmol), and Cs₂CO₃ (391 mg, 1.2 mmol) in dioxane (6 mL) and H₂O (1.5 mL) was stirred at 90° C. under nitrogen for 4 h. The reaction mixture was then concentrated and purified by flash chromatography (PE/EA: 100/0 to 80/20) to yield compound 7 (164 mg) in 80% yield. LCMS (ESI) m/z: 341.2 [M+H⁺].

(4-(Benzo[d]thiazol-7-yl)phenyl)methanamine 8. A mixture of compound 7 (163.6 mg, 0.48 mmol) in DCM (4 mL) and HCl (1 mL, 4 M in 1,4-dioxane) was stirred at 40° C. for 1 h. The reaction mixture was then concentrated and treated with DCM (5 mL) and Et₃N (2 mL). The resulting mixture was stirred at room temperature for another 1 h and then concentrated to yield compound 8 in a quantitative yield, which was used directly in the next step without further purification. LCMS (ESI) m/z: 241.1 [M+H⁺].

1-(4-(Benzo[d]thiazol-7-yl)benzyl)-3-(2-((trimethylsilyl)ethynyl)thiazol-4-yl)urea 9. A mixture of compound 3 (60.4 mg, 0.17 mmol), compound 8 (40.2 mg, 0.17 mmol), DMAP (20.4 mg, 0.17 mmol), and Et₃N (16.9 mg, 0.17 mmol) in MeCN (5 mL) was stirred at 85° C. for 3 h. The reaction solution was then concentrated and purified by flash chromatography (PE/EA: 100/0 to 60/40) to yield compound 9 (79 mg) in 99% yield. LCMS (ESI) m/z: 463.1 [M+H⁺].

1-(4-(Benzo[d]thiazol-7-yl)benzyl)-3-(2-ethynylthiazol-4-yl)urea A6. A mixture of compound 9 (79 mg, 0.17 mmol) and K₂CO₃ (23.6 mg, 0.17 mmol) in MeOH (3 mL) was stirred at room temperature for 1 h. The reaction mixture was then concentrated and purified by prep-HPLC to yield compound A6 (30 mg) in 45% yield. ¹H NMR (400 MHz, DMSO-d₆) δ 9.53 (s, 1H), 9.45 (s, 1H), 8.10 (dd, J=8.1, 1.0 Hz, 1H), 7.70 (d, J=8.2 Hz, 2H), 7.66 (d, J=7.9 Hz, 1H), 7.58 (d, J=6.9 Hz, 1H), 7.48 (d, J=8.2 Hz, 2H), 7.34 (s, 1H), 6.91 (t, J=6.0 Hz, 1H), 4.90 (s, 1H), 4.41 (d, J=5.9 Hz, 2H); MS (ESI) m/z: 291.0 [M+H⁺].

The following compounds were prepared similarly.

1-(4-(1,1-Dioxidobenzo[b]thiophen-7-yl)benzyl)-3-(2-ethynylthiazol-4-yl)urea A1. ¹H NMR (400 MHz, DMSO-d₆) δ 9.56 (s, 1H), 7.74 (t, J=7.6 Hz, 1H), 7.67 (dd, J=10.8, 7.6 Hz, 3H), 7.58 (dd, J=7.5, 4.3 Hz, 2H), 7.43 (d, J=8.2 Hz, 2H), 7.36 (dd, J=12.7, 5.8 Hz, 2H), 6.95 (d, J=5.8 Hz, 1H), 4.88 (d, J=4.9 Hz, 1H), 4.40 (d, J=5.9 Hz, 2H); MS (ESI) m/z: 422.2 [M+H⁺].

1-(2-Ethynylthiazol-4-yl)-3-(4-(3-hydroxy-1,1-dioxido-2,3-dihydrobenzo[b]-thiophen-7-yl)benzyl)urea A2. ¹H NMR (400 MHz, DMSO-d₆) δ 9.53 (s, 1H), 7.80 (t, J=7.6 Hz, 1H), 7.68 (d, J=7.7 Hz, 1H), 7.60 (d, J=8.2 Hz, 2H), 7.54 (d, J=7.3 Hz, 1H), 7.39 (d, J=8.2 Hz, 2H), 7.34 (s, 1H), 6.91 (t, J=6.0 Hz, 1H), 6.36 (d, J=5.7 Hz, 1H), 5.41 (q, J=6.0 Hz, 1H), 4.88 (s, 1H), 4.39 (d, J=5.9 Hz, 2H), 3.99 (dd, J=13.3, 6.9 Hz, 1H); MS (ESI) m/z: 440.2 [M+H⁺].

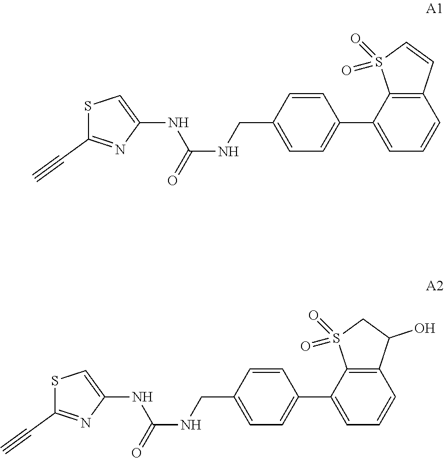 [SMILES: C#Cc1nc(NC(=O)NCc2ccc(-c3cccc4c3S(=O)(=O)C=C4)cc2)cs1 ; C#Cc1nc(NC(=O)NCc2ccc(-c3cccc4c3S(=O)(=O)CC4O)cc2)cs1]

1-(4-(1,1-Dioxido-3-oxo-2,3-dihydrobenzo[b]thiophen-7-yl)benzyl)-3-(2-ethynyl-thiazol-4-yl)urea A3. ¹H NMR (400 MHz, DMSO-d₆) δ 9.54 (s, 1H), 7.95 (s, 3H), 7.69 (d, J=7.6 Hz, 2H), 7.43 (d, J=7.8 Hz, 2H), 7.34 (s, 1H), 6.91 (d, J=5.7 Hz, 1H), 4.89 (s, 1H), 4.63 (s, 2H), 4.41 (d, J=5.7 Hz, 2H); MS (ESI) m/z: 438.0 [M+H⁺].

1-(2-Ethynylthiazol-4-yl)-3-(4-(8-oxo-7,8-dihydro-6H-thiazolo[5,4-e]isoindol-5-yl)benzyl)urea A4. ¹H NMR (400 MHz, DMSO-d₆) δ 9.55 (d, J=3.3 Hz, 2H), 9.01 (s, 1H), 8.33 (s, 1H), 7.71 (d, J=8.3 Hz, 2H), 7.45 (d, J=8.3 Hz, 2H), 7.34 (s, 1H), 6.92 (t, 1H), 4.90 (s, 1H), 4.66 (s, 2H), 4.41 (d, J=5.9 Hz, 2H).

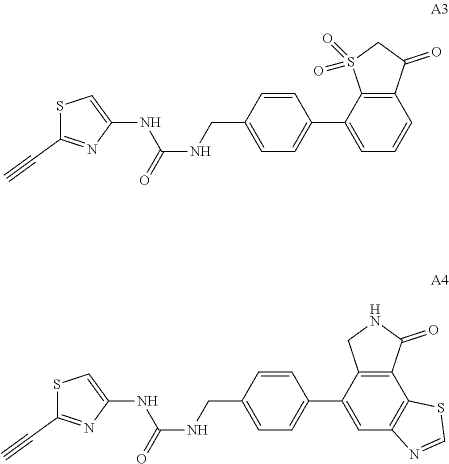 [SMILES: C#Cc1nc(NC(=O)NCc2ccc(-c3cccc4c3S(=O)(=O)CC4=O)cc2)cs1 ; C#Cc1nc(NC(=O)NCc2ccc(-c3cc4ncsc4c4c3CNC4=O)cc2)cs1]

(S)-1-(2-Ethynylthiazol-4-yl)-3-((3′-(3-hydroxypyrrolidin-1-yl)-[1,1′-biphenyl]-4-yl)methyl)urea A5. ¹H NMR (400 MHz, DMSO-d₆) δ 9.49 (s, 1H), 7.59 (d, J=8.2 Hz, 2H), 7.35 (d, J=8.2 Hz, 2H), 7.32 (s, 1H), 7.22 (t, J=7.9 Hz, 1H), 6.84 (dd, J=11.5, 6.7 Hz, 2H), 6.69 (d, J=1.9 Hz, 1H), 6.50 (dd, J=8.2, 1.9 Hz, 1H), 4.95 (d, J=3.7 Hz, 1H), 4.89 (s, 1H), 4.41 (s, 1H), 4.35 (d, J=5.9 Hz, 2H), 3.46 (dd, J=10.1, 5.0 Hz, 1H), 3.38 (d, J=7.3 Hz, 2H), 3.12 (dd, J=9.9, 1.8 Hz, 1H), 2.10-1.99 (m, 1H), 1.91 (d, J=3.5 Hz, 1H); MS (ESI) m/z: 419.2 [M+H⁺].

1-(2-Ethynylthiazol-4-yl)-3-((3′-(pyrrolidin-1-yl)-[1,1′-biphenyl]-4-yl)methyl)urea A7. ¹H NMR (400 MHz, DMSO-d₆) δ 9.49 (s, 1H), 7.59 (d, J=8.3 Hz, 2H), 7.35 (d, J=8.3 Hz, 2H), 7.32 (s, 1H), 7.22 (t, J=7.9 Hz, 1H), 6.84 (dd, J=10.5, 5.2 Hz, 2H), 6.73-6.69 (m, 1H), 6.52 (dd, J=8.2, 1.8 Hz, 1H), 4.89 (s, 1H), 4.34 (d, J=5.9 Hz, 2H), 3.28 (t, J=6.6 Hz, 4H), 2.01-1.92 (m, 4H); MS (ESI) m/z: 403.1 [M+H⁺].

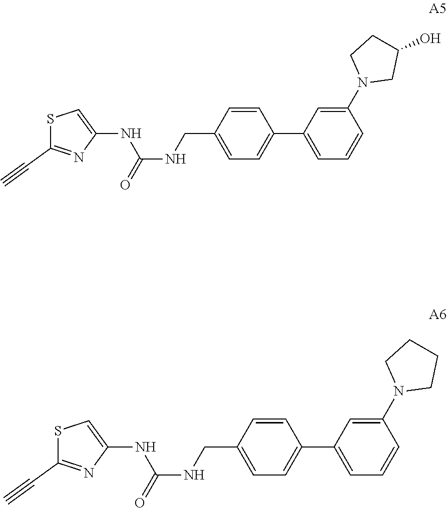 [SMILES: C#Cc1nc(NC(=O)NCc2ccc(-c3cccc(N4CC[C@H](O)C4)c3)cc2)cs1 ; C#Cc1nc(NC(=O)NCc2ccc(-c3cccc(N4CCCC4)c3)cc2)cs1]

1-([1,1′-Biphenyl]-4-ylmethyl)-3-(2-ethynylthiazol-4-yl)urea A8. ¹H NMR (400 MHz, DMSO-d₆) δ 9.50 (s, 1H), 7.66-7.62 (m, 4H), 7.48-7.44 (m, 2H), 7.40-7.32 (m, 4H), 6.85 (t, J=6.0 Hz, 1H), 4.89 (s, 1H), 4.36 (d, J=5.9 Hz, 2H); MS (ESI) m/z: 334.1 [M+H⁺].

1-(2-Ethynylthiazol-4-yl)-3-(4-(4-hydroxyquinazolin-5-yl)benzyl)urea A9. ¹H NMR (400 MHz, DMSO-d₆) δ 11.99 (d, J=3.1 Hz, 1H), 9.49 (s, 1H), 8.05 (d, J=3.6 Hz, 1H), 7.77 (t, J=7.8 Hz, 1H), 7.66 (dd, J=8.1, 1.2 Hz, 1H), 7.34 (s, 1H), 7.26 (s, 4H), 7.22 (dd, J=7.4, 1.2 Hz, 1H), 6.85 (t, J=5.9 Hz, 1H), 4.89 (s, 1H), 4.38 (d, J=5.9 Hz, 2H); MS (ESI) m/z: 402.1 [M+H⁺].

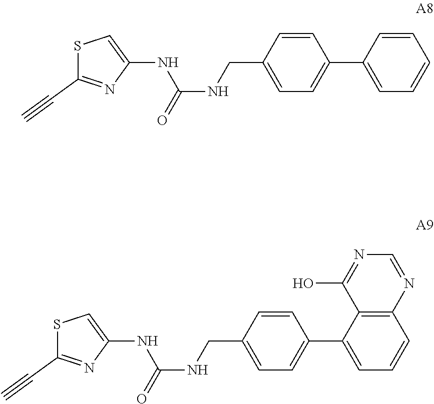 [SMILES: C#Cc1nc(NC(=O)NCc2ccc(-c3ccccc3)cc2)cs1 ; C#Cc1nc(NC(=O)NCc2ccc(-c3cccc4ncnc(O)c34)cc2)cs1]

4′-((3-(2-Ethynylthiazol-4-yl)ureido)methyl)-[1,1′-biphenyl]-3-carboxamide A10. ¹H NMR (400 MHz, DMSO-d₆) δ 9.50 (s, 1H), 8.14 (t, J=1.6 Hz, 1H), 8.09 (s, 1H), 7.86-7.78 (m, 2H), 7.70 (d, J=8.3 Hz, 2H), 7.53 (t, J=7.7 Hz, 1H), 7.41 (d, J=8.2 Hz, 3H), 7.33 (s, 1H), 6.86 (t, J=5.9 Hz, 1H), 4.89 (s, 1H), 4.37 (d, J=5.9 Hz, 2H); MS (ESI) m/z: 377.1 [M+H⁺].

1-(2-Ethynylthiazol-4-yl)-3-(4-(3-methyl-4-oxo-3,4-dihydroquinazolin-5-yl)-benzyl)urea A11. ¹H NMR (400 MHz, DMSO-d₆) δ 9.49 (s, 1H), 8.38 (s, 1H), 7.82-7.73 (m, 1H), 7.67 (dd, J=8.1, 1.2 Hz, 1H), 7.35 (s, 1H), 7.31-7.17 (m, 5H), 6.86 (t, J=5.9 Hz, 1H), 4.89 (s, 1H), 4.38 (d, J=5.9 Hz, 2H), 3.35 (s, 3H); MS (ESI) m/z: 416.1 [M+H⁺].

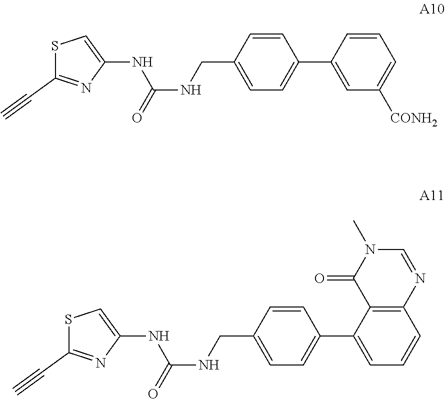 [SMILES: C#Cc1nc(NC(=O)NCc2ccc(-c3cccc(C(N)=O)c3)cc2)cs1 ; C#Cc1nc(NC(=O)NCc2ccc(-c3cccc4ncn(C)c(=O)c34)cc2)cs1]

1-(2-Ethynylthiazol-4-yl)-3-(4-(6-fluorobenzo[d]thiazol-5-yl)benzyl)urea A12. ¹H NMR (400 MHz, DMSO-d₆) δ 9.52 (s, 1H), 9.41 (s, 1H), 8.15 (ddd, J=13.3, 9.6, 6.4 Hz, 2H), 7.61 (dd, J=8.2, 1.6 Hz, 2H), 7.43 (d, J=8.2 Hz, 2H), 7.34 (d, J=3.3 Hz, 1H), 6.89 (t, J=6.0 Hz, 1H), 4.90 (s, 1H), 4.39 (d, J=5.9 Hz, 2H); MS (ESI) m/z: 401.1 [M+H⁺].

4′-((3-(2-Ethynylthiazol-4-yl)ureido)methyl)-N-methyl-[1,1′-biphenyl]-2-sulfonamide A13. ¹H NMR (400 MHz, DMSO-d₆) δ 9.99 (s, 1H), 8.54 (s, 1H), 7.94-7.87 (m, 1H), 7.65 (td, J=7.5, 1.4 Hz, 1H), 7.58 (td, J=7.6, 1.4 Hz, 1H), 7.39-7.24 (m, 6H), 7.11 (s, 1H), 4.88 (s, 1H), 4.36 (d, J=5.8 Hz, 2H), 2.36 (d, J=3.5 Hz, 3H); MS (ESI) m/z: 427.1 [M+H⁺].

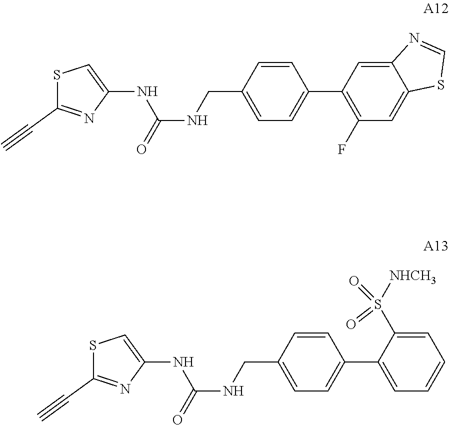 [SMILES: C#Cc1nc(NC(=O)NCc2ccc(-c3cc4ncsc4cc3F)cc2)cs1 ; C#Cc1nc(NC(=O)NCc2ccc(-c3ccccc3S(=O)(=O)NC)cc2)cs1]

4′-((3-(2-Ethynylthiazol-4-yl)ureido)methyl)-N-methyl-[1,1′-biphenyl]-3-carboxamide A14. ¹H NMR (400 MHz, DMSO-d₆) δ 9.51 (s, 1H), 8.54 (d, J=4.6 Hz, 1H), 8.10 (t, J=1.6 Hz, 1H), 7.80 (dt, J=7.9, 1.7 Hz, 2H), 7.70 (d, J=8.3 Hz, 2H), 7.54 (t, J=7.7 Hz, 1H), 7.41 (d, J=8.3 Hz, 2H), 7.32 (s, 1H), 6.87 (t, J=5.9 Hz, 1H), 4.90 (s, 1H), 4.37 (d, J=5.9 Hz, 2H), 2.81 (d, J=4.5 Hz, 3H); MS (ESI) m/z: 391.1 [M+H⁺].

1-(4-(1,5-Dimethyl-1H-indazol-4-yl)benzyl)-3-(2-ethynylthiazol-4-yl)urea A15. ¹H NMR (400 MHz, DMSO-d₆) δ 9.52 (s, 1H), 7.55-7.52 (m, 2H), 7.45-7.42 (m, 2H), 7.40-7.38 (m, 2H), 7.34 (t, J=4.4 Hz, 2H), 6.91 (t, J=5.9 Hz, 1H), 4.90 (s, 1H), 4.42 (d, J=5.9 Hz, 2H), 4.03 (s, 3H), 2.28 (s, 3H); MS (ESI) m/z: 402.2 [M+H⁺].

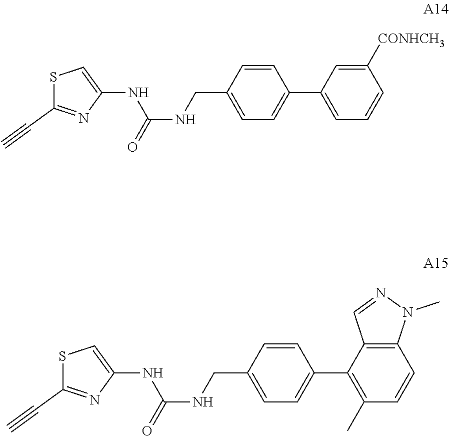

1-(2-Ethynylthiazol-4-yl)-3-(4-(1-methyl-1H-indazol-4-yl)benzyl)urea A16. ¹H NMR (400 MHz, DMSO-d₆) δ 9.52 (s, 1H), 8.13 (d, J=0.9 Hz, 1H), 7.72-7.69 (m, 2H), 7.65-7.62 (m, 1H), 7.50-7.44 (m, 3H), 7.34 (s, 1H), 7.26-7.24 (m, 1H), 6.90 (t, J=5.9 Hz, 1H), 4.90 (s, 1H), 4.40 (d, J=5.9 Hz, 2H), 4.09 (s, 3H); MS (ESI) m/z: 388.1 [M+H⁺].

7-(4-((3-(2-Ethynylthiazol-4-yl)ureido)methyl)phenyl)-N-methylbenzo[d]-thiazole-6-carboxamide A17. ¹H NMR (400 MHz, DMSO-d₆) δ 9.52 (s, 1H), 9.45 (s, 1H), 8.10 (dd, J=8.3, 3.7 Hz, 2H), 7.60 (d, J=8.3 Hz, 1H), 7.45 (d, J=8.2 Hz, 2H), 7.39 (d, J=8.2 Hz, 2H), 7.35 (s, 1H), 6.90 (t, J=5.9 Hz, 1H), 4.90 (s, 1H), 4.41 (d, J=5.9 Hz, 2H), 2.57 (d, J=4.6 Hz, 3H); MS (ESI) m/z: 448.0 [M+H⁺].

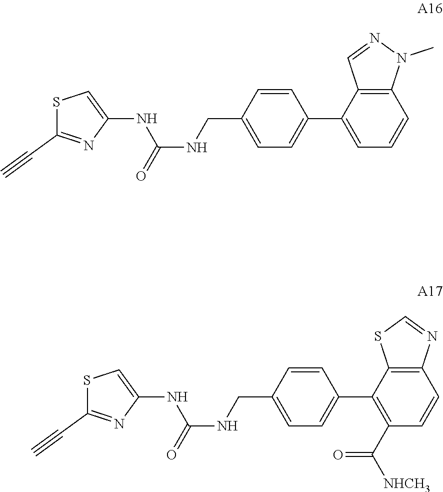

1-(2-Ethynylthiazol-4-yl)-3-(4-(1-hydroxyisoquinolin-8-yl)benzyl)urea A18. ¹H NMR (400 MHz, DMSO-d₆) δ 10.91 (d, J=5.6 Hz, 1H), 9.48 (s, 1H), 7.75-7.54 (m, 2H), 7.35 (s, 1H), 7.22 (q, J=8.3 Hz, 4H), 7.17-7.11 (m, 2H), 6.85 (s, 1H), 6.56 (dd, J=7.0, 1.2 Hz, 1H), 4.89 (s, 1H), 4.37 (d, J=5.9 Hz, 2H); MS (ESI) m/z: 401.1 [M+H⁺].

7-(4-((3-(2-Ethynylthiazol-4-yl)ureido)methyl)phenyl)benzo[d]thiazole-6-carboxamide A19. ¹H NMR (400 MHz, DMSO-d₆) δ 9.54 (s, 1H), 9.44 (s, 1H), 8.10 (d, =8.3 Hz, 1H), 7.64 (d, J=8.3 Hz, 2H), 7.48 (d, J=8.3 Hz, 2H), 7.40 (d, J=8.3 Hz, 2H), 7.33 (d, J=11.3 Hz, 2H), 6.93 (t, J=6.0 Hz, 1H), 4.90 (s, 1H), 4.40 (d, J=5.9 Hz, 2H); MS (ESI) m/z: 434.1 [M+H⁺].

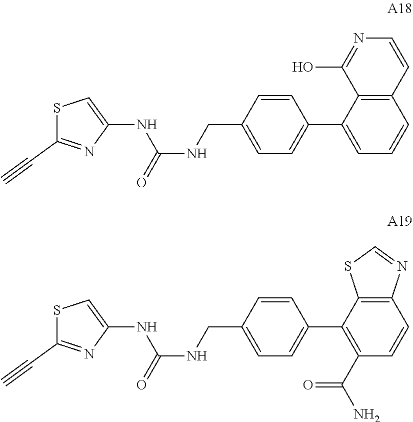 [SMILES: C#Cc1nc(NC(=O)NCc2ccc(-c3cccc4ccnc(O)c34)cc2)cs1 ; C#Cc1nc(NC(=O)NCc2ccc(-c3c(C(N)=O)ccc4ncsc34)cc2)cs1]

4-(4-((3-(2-Ethynylthiazol-4-yl)ureido)methyl)phenyl)-1-methyl-1H-indazole-6-carboxamide A20. ¹H NMR (400 MHz, DMSO-d₆) δ 9.53 (s, 1H), 8.22-8.12 (m, 3H), 7.76 (dd, J=4.7, 3.5 Hz, 3H), 7.49 (t, J=6.8 Hz, 3H), 7.34 (s, 1H), 6.91 (t, J=6.0 Hz, 1H), 4.90 (s, 1H), 4.41 (d, J=5.9 Hz, 2H), 4.14 (s, 3H); MS (ESI) m/z: 431.1 [M+H⁺].

4-(4-((3-(2-Ethynylthiazol-4-yl)ureido)methyl)phenyl)-N,1-dimethyl-1H-indazole-6-carboxamide A21. ¹H NMR (400 MHz, DMSO-d₆) δ 9.53 (s, 1H), 8.62 (d, J=4.5 Hz, 1H), 8.20 (d, J=0.9 Hz, 1H), 8.13 (s, 1H), 7.76 (d, J=8.2 Hz, 2H), 7.71 (d, J=1.2 Hz, 1H), 7.48 (d, J=8.2 Hz, 2H), 7.34 (s, 1H), 6.90 (t, J=6.0 Hz, 1H), 4.90 (s, 1H), 4.42 (d, J=5.9 Hz, 2H), 4.13 (s, 3H), 2.85 (d, J=4.5 Hz, 3H); MS (ESI) m/z: 445.1 [M+H⁺].

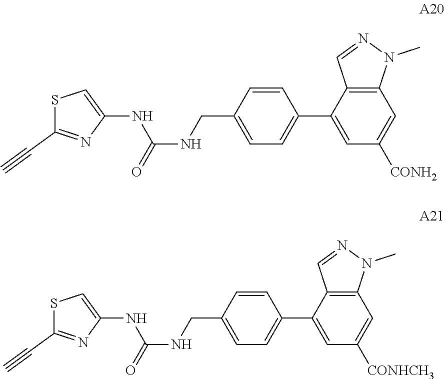

Methyl 4-(4-((3-(2-ethynylthiazol-4-yl)ureido)methyl)phenyl)-1-methyl-1H-indazole-6-carboxylate A22. ¹H NMR (400 MHz, DMSO-d₆) δ 9.55 (s, 1H), 8.30 (dd, J=4.8, 3.8 Hz, 1H), 8.24 (dd, J=3.1, 1.0 Hz, 1H), 7.79-7.70 (m, 3H), 7.49 (d, J=8.2 Hz, 2H), 7.34 (s, 1H), 6.92 (t, J=6.0 Hz, 1H), 4.90 (s, 1H), 4.42 (d, J=6.0 Hz, 2H), 4.18 (s, 3H), 3.94 (s, 3H); MS (ESI) m/z: 446.1 [M+H⁺].

4-(4-((3-(2-Ethynylthiazol-4-yl)ureido)methyl)phenyl)-N,N,1-trimethyl-1H-indazole-6-carboxamide A23. ¹H NMR (400 MHz, DMSO-d₆) δ 9.54 (s, 1H), 8.19 (d, J=0.9 Hz, 1H), 7.73 (d, J=8.0 Hz, 3H), 7.46 (d, J=8.2 Hz, 2H), 7.34 (s, 1H), 7.22 (d, J=1.1 Hz, 1H), 6.92 (t, J=6.0 Hz, 1H), 4.90 (s, 1H), 4.41 (d, J=5.9 Hz, 2H), 4.11 (s, 3H), 3.01 (d, J=23.0 Hz, 6H); MS (ESI) m/z: 459.2 [M+H⁺].

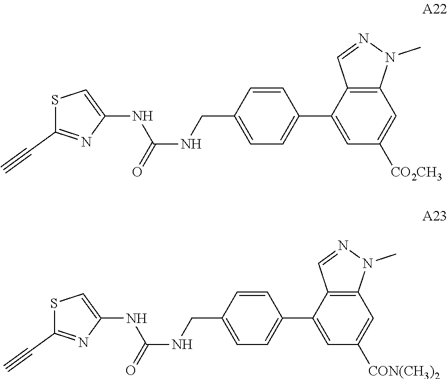

1-(2-Ethynylthiazol-4-yl)-3-(4-(1-methyl-3-oxo-2,3-dihydro-1H-indazol-4-yl)-benzyl)urea A24. ¹H NMR (400 MHz, DMSO-d₆) δ 10.47 (s, 1H), 9.48 (s, 1H), 7.52 (d, J=8.2 Hz, 2H), 7.41 (dd, J=8.5, 1.1 Hz, 1H), 7.38 (d, J=6.6 Hz, 1H), 7.34 (t, J=4.0 Hz, 3H), 6.92 (dd, J=6.6, 1.2 Hz, 1H), 6.87 (t, J=5.9 Hz, 1H), 4.89 (s, 1H), 4.37 (d, J=5.8 Hz, 2H), 3.80 (s, 3H); MS (ESI) m/z: 404.1 [M+H⁺].

(S)-1-(1-(4-(Benzo[d]thiazol-7-yl)phenyl)ethyl)-3-(2-ethynylthiazol-4-yl)urea A25. ¹H NMR (400 MHz, DMSO-d₆) δ 9.45 (s, 1H), 9.30 (s, 1H), 8.10 (dd, J=8.1, 1.1 Hz, 1H), 7.73-7.69 (m, 2H), 7.69-7.64 (m, 1H), 7.58 (d, J=6.7 Hz, 1H), 7.52 (d, J=8.2 Hz, 2H), 7.29 (s, 1H), 6.93 (d, J=7.7 Hz, 1H), 4.95-4.90 (m, 1H), 4.90 (s, 1H), 1.46 (d, J=7.0 Hz, 3H); MS (ESI) m/z: 405.1 [M+H⁺].

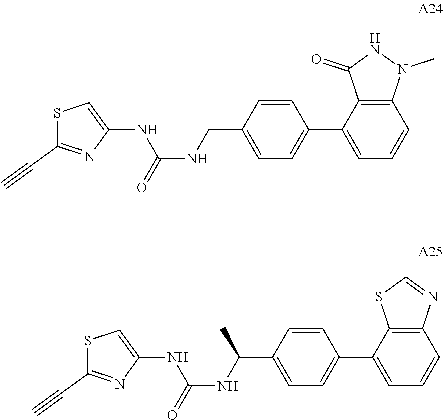

(R)-1-(1-(4-(Benzo[d]thiazol-7-yl)phenyl)ethyl)-3-(2-ethynylthiazol-4-yl)urea A26. ¹H NMR (400 MHz, DMSO-d₆) δ 9.45 (s, 1H), 9.30 (s, 1H), 8.10 (dd, J=8.1, 1.0 Hz, 1H), 7.71 (d, J=8.3 Hz, 2H), 7.70-7.64 (m, 1H), 7.58 (d, J=6.7 Hz, 1H), 7.52 (dd, J=8.3, 4.5 Hz, 2H), 7.33-7.25 (m, 1H), 6.93 (d, J=7.7 Hz, 1H), 5.16-4.58 (m, 2H), 1.46 (d, J=7.0 Hz, 3H); MS (ESI) m/z: 405.0 [M+H⁺].

1-(4-(2,2-Difluoro-1,1-dioxido-3-oxo-2,3-dihydrobenzo[b]thiophen-7-yl)benzyl)-3-(2-ethynylthiazol-4-yl)urea A27. ¹H NMR (400 MHz, DMSO-d₆) δ 9.54 (s, 1H), 8.18 (s, 2H), 7.94 (t, J=7.7 Hz, 1H), 7.87 (dd, J=7.8, 1.1 Hz, 1H), 7.71 (dd, J=7.6, 1.1 Hz, 1H), 7.60 (d, J=8.3 Hz, 2H), 7.45 (d, J=8.3 Hz, 2H), 7.34 (s, 1H), 6.92 (t, J=5.9 Hz, 1H), 4.89 (s, 1H), 4.41 (d, J=5.9 Hz, 2H); MS (ESI) m/z: 492.0 [M+H⁺].

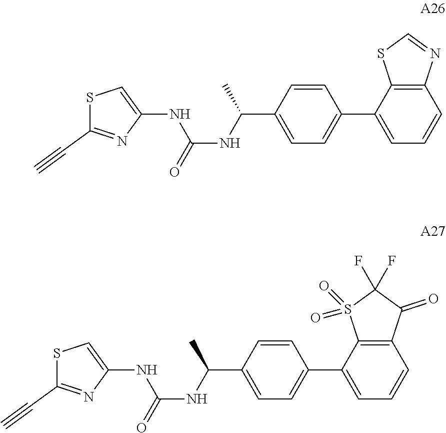 [SMILES: C#Cc1nc(NC(=O)N[C@H](C)c2ccc(-c3cccc4ncsc34)cc2)cs1 ; C#Cc1nc(NC(=O)N[C@@H](C)c2ccc(-c3cccc4c3S(=O)(=O)C(F)(F)C4=O)cc2)cs1]

1-(2-Ethynylthiazol-4-yl)-3-((3′-(oxetan-3-ylamino)-[1,1′-biphenyl]-4-yl)methyl)-urea A28. ¹H NMR (400 MHz, DMSO-d₆) δ 9.49 (s, 1H), 7.54 (d, J=8.3 Hz, 2H), 7.35 (d, J=8.3 Hz, 2H), 7.32 (s, 1H), 7.16 (t, J=7.8 Hz, 1H), 6.85 (d, J=6.5 Hz, 2H), 6.69 (t, J=1.9 Hz, 1H), 6.48-6.42 (m, 2H), 4.89 (s, 1H), 4.86 (t, J=6.5 Hz, 2H), 4.65-4.55 (m, 1H), 4.42 (t, J=6.1 Hz, 2H), 4.34 (d, J=5.9 Hz, 2H); MS (ESI) m/z: 405.1 [M+H⁺].

1-((2′-Cyano-[1,1′-biphenyl]-4-yl)methyl)-3-(2-ethynylthiazol-4-yl)urea A29. ¹H NMR (400 MHz, DMSO-d₆) δ 9.55 (s, 1H), 7.95 (d, J=7.7, 1H), 7.79 (t, J=7.6, 1H), 7.69-7.51 (m, 4H), 7.45 (d, J=8.0, 2H), 7.33 (s, 1H), 6.93 (s, 1H), 4.89 (s, 1H), 4.41 (d, J=5.6, 2H); MS (ESI) m/z: 359.1 [M+H⁺].

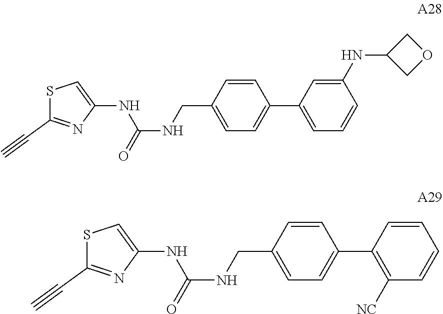 [SMILES: C#Cc1nc(NC(=O)NCc2ccc(-c3cccc(NC4COC4)c3)cc2)cs1 ; C#Cc1nc(NC(=O)NCc2ccc(-c3ccccc3C#N)cc2)cs1]

1-(2-Ethynylthiazol-4-yl)-3-(4-(pyrrolidin-1-yl)benzyl)urea A30. ¹H NMR (400 MHz, DMSO-d₆) δ 9.33 (s, 1H), 7.31 (s, 1H), 7.09 (d, J=8.6 Hz, 2H), 6.58 (t, J=5.7 Hz, 1H), 6.49 (d, J=8.6 Hz, 2H), 4.88 (s, 1H), 4.16 (d, J=5.7 Hz, 2H), 3.18 (dd, J=8.5, 4.7 Hz, 4H), 2.02-1.80 (m, 4H); MS (ESI) m/z: 160.2 [Fragment+H⁺].

1-(4-((3-(2-Ethynylthiazol-4-yl)ureido)methyl)phenyl)pyrrolidine-2-carboxamide A31. ¹H NMR (400 MHz, DMSO-d₆) δ 9.33 (s, 1H), 7.37-7.24 (m, 2H), 7.10 (d, J=8.6 Hz, 2H), 7.00 (s, 1H), 6.58 (s, 1H), 6.44 (d, J=8.6 Hz, 2H), 4.88 (s, 1H), 4.16 (d, J=5.6 Hz, 2H), 3.91-3.81 (m, 1H), 3.54 (t, J=6.5 Hz, 1H), 3.15 (dd, J=15.5, 8.1 Hz, 1H), 2.16 (ddd, J=34.1, 20.7, 12.5 Hz, 1H), 2.02-1.84 (m, 3H).

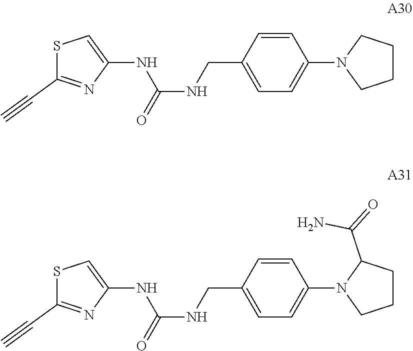 [SMILES: C#Cc1nc(NC(=O)NCc2ccc(N3CCCC3)cc2)cs1 ; C#Cc1nc(NC(=O)NCc2ccc(N3CCCC3C(N)=O)cc2)cs1]

(S)-1-(4-(2-Cyanopyrrolidin-1-yl)benzyl)-3-(2-ethynylthiazol-4-yl)urea A32. ¹H NMR (400 MHz, DMSO-d₆) δ 9.37 (s, 1H), 7.31 (s, 1H), 7.20 (d, J=8.6 Hz, 2H), 6.70 (d, J=8.7 Hz, 2H), 6.66 (t, J=6.2 Hz, 1H), 4.89 (s, 1H), 4.86-4.76 (m, 1H), 4.21 (d, J=5.8 Hz, 2H), 3.30 (d, J=4.0 Hz, 1H), 2.31-2.21 (m, 2H), 2.10 (dd, J=15.4, 7.3 Hz, 2H).

(R)-1-(4-(2-Cyanopyrrolidin-1-yl)benzyl)-3-(2-ethynylthiazol-4-yl)urea A33. ¹H NMR (400 MHz, DMSO-d₆) δ 9.36 (s, 1H), 7.31 (s, 1H), 7.20 (d, J=8.0 Hz, 2H), 6.73-6.63 (m, 3H), 4.87 (s, 1H), 4.82 (s, 1H), 4.21 (d, J=5.5 Hz, 2H), 2.27 (d, J=6.0 Hz, 2H), 2.14-1.93 (m, 4H); MS (ESI) m/z: 185.1 [Fragment+H⁺].

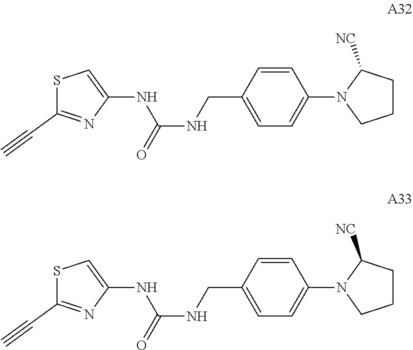 [SMILES: C#Cc1nc(NC(=O)NCc2ccc(N3CCC[C@H]3C#N)cc2)cs1 ; C#Cc1nc(NC(=O)NCc2ccc(N3CCC[C@@H]3C#N)cc2)cs1]

1-(2-(3-(2-Cyanophenyl)azetidin-1-yl)-2-oxoethyl)-3-(2-ethynylthiazol-4-yl)urea A34. ¹H NMR (400 MHz, DMSO-d₆) δ 9.66 (s, 1H), 7.84 (d, J=7.7 Hz, 1H), 7.82-7.68 (m, 2H), 7.55-7.45 (m, 1H), 7.29 (s, 1H), 6.63 (t, J=4.8 Hz, 1H), 4.90 (s, 1H), 4.66 (t, J=8.5 Hz, 1H), 4.35 (dt, J=14.5, 8.6 Hz, 2H), 4.28-4.17 (m, 1H), 4.02 (dd, J=9.0, 6.2 Hz, 1H), 3.83 (d, J=5.0 Hz, 2H); MS (ESI) m/z: 366.1 [M+H⁺].

1-(2-(4-(2-Cyanophenyl)piperidin-1-yl)-2-oxoethyl)-3-(2-ethynylthiazol-4-yl)urea A35. ¹H NMR (400 MHz, DMSO-d₆) δ 9.76 (s, 1H), 7.81 (d, J=7.8 Hz, 1H), 7.68 (t, J=7.1 Hz, 1H), 7.54 (d, J=7.8 Hz, 1H), 7.43 (t, J=7.2 Hz, 1H), 7.29 (s, 1H), 6.69 (s, 1H), 4.90 (s, 1H), 4.56 (d, J=12.9 Hz, 1H), 4.12-4.02 (m, 2H), 3.92 (d, J=13.1 Hz, 1H), 3.15 (dd, J=15.5, 11.9 Hz, 2H), 2.72 (dd, J=25.4, 14.0 Hz, 1H), 1.85-1.70 (m, 3H), 1.58 (dd, J=12.9, 3.7 Hz, 1H); MS (ESI) m/z: 394.1 [M+H⁺].

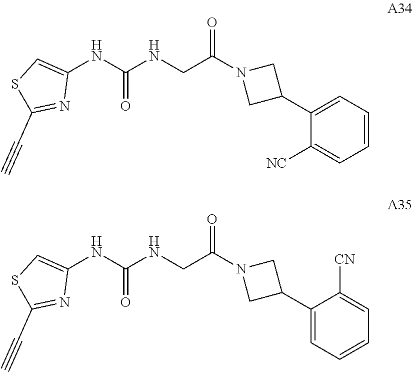 [SMILES: C#Cc1nc(NC(=O)NCC(=O)N2CC(c3ccccc3C#N)C2)cs1 ; C#Cc1nc(NC(=O)NCC(=O)N2CC(c3ccccc3C#N)C2)cs1]

1-(2-(4-(2-Cyanophenyl)piperazin-1-yl)-2-oxoethyl)-3-(2-ethynylthiazol-4-yl)urea A36. ¹H NMR (400 MHz, DMSO-d₆) δ 9.74 (s, 1H), 7.74 (dd, J=7.7, 1.6 Hz, 1H), 7.63 (ddd, J=9.0, 7.5, 1.6 Hz, 1H), 7.29 (s, 1H), 7.19 (d, J=8.1 Hz, 1H), 7.14 (td, J=7.6, 0.9 Hz, 1H), 6.66 (t, J=4.7 Hz, 1H), 4.90 (s, 1H), 4.08 (d, J=4.9 Hz, 2H), 3.64 (d, J=21.6 Hz, 4H), 3.16 (d, J=22.5 Hz, 4H); MS (ESI) m/z: 395.2 [M+H⁺].

1-((1-(2-Cyanophenyl)piperidin-4-yl)methyl)-3-(2-ethynylthiazol-4-yl)urea A37. ¹H NMR (400 MHz, DMSO-d₆) δ 9.33 (s, 1H), 7.67 (dd, J=7.7, 1.6 Hz, 1H), 7.63-7.52 (m, 1H), 7.30 (s, 1H), 7.15 (d, J=8.3 Hz, 1H), 7.05 (t, J=7.5 Hz, 1H), 6.47 (t, J=5.9 Hz, 1H), 4.89 (s, 1H), 3.51 (d, J=12.0 Hz, 2H), 3.09 (t, J=6.2 Hz, 2H), 2.76 (t, J=11.1 Hz, 2H), 1.77 (d, J=10.7 Hz, 2H), 1.58 (d, J=11.3 Hz, 1H), 1.35 (qd, J=12.2, 3.7 Hz, 2H); MS (ESI) m/z: 366.2

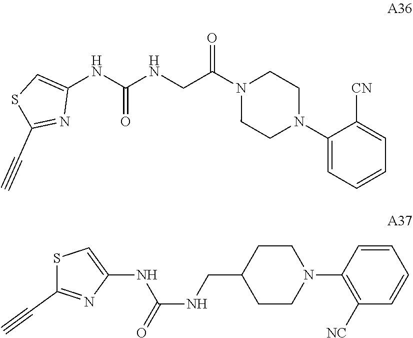 [SMILES: C#Cc1nc(NC(=O)NCC(=O)N2CCN(c3ccccc3C#N)CC2)cs1 ; C#Cc1nc(NC(=O)NCC2CCN(c3ccccc3C#N)CC2)cs1]

1-(Adamantan-1-ylmethyl)-3-(2-ethynylthiazol-4-yl)urea A38. ¹H NMR (400 MHz, DMSO-d₆) δ 9.25 (s, 1H), 7.26 (s, 1H), 6.35 (t, J=6.1 Hz, 1H), 4.88 (s, 1H), 2.81 (d, J=6.1 Hz, 2H), 2.07-1.83 (m, 3H), 1.73-1.53 (m, 6H), 1.44 (d, J=2.8 Hz, 6H); MS (ESI) m/z: 316.1 [M+H⁺].

1-(1-(4-(Benzo[d]thiazol-7-yl)phenyl)piperidin-4-yl)-3-(2-ethynylthiazol-4-yl)urea A39.

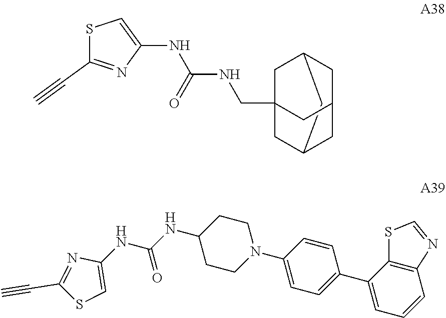 [SMILES: C#Cc1nc(NC(=O)NCC23CC4CC(CC(C4)C2)C3)cs1 ; C#Cc1nc(NC(=O)NC2CCN(c3ccc(-c4cccc5ncsc45)cc3)CC2)cs1]

(R)-1-(1-(4-(Benzo[d]thiazol-7-yl)phenyl)-2-hydroxyethyl)-3-(2-ethynylthiazol-4-yl)urea A40. ¹H NMR (400 MHz, DMSO-d₆) δ 9.52 (s, 1H), 9.45 (s, 1H), 8.10 (dd, J=8.1, 1.0 Hz, 1H), 7.70 (t, J=5.2 Hz, 2H), 7.69-7.64 (m, 1H), 7.58 (d, J=7.3 Hz, 1H), 7.49 (d, J=8.2 Hz, 2H), 7.28 (s, 1H), 7.11 (d, J=7.8 Hz, 1H), 5.10 (t, J=5.1 Hz, 1H), 4.90 (s, 1H), 4.85 (dd, J=12.7, 5.1 Hz, 1H), 3.78-3.62 (m, 2H); MS (ESI) m/z: 421.1 [M+H⁺].

(S)-1-(1-(4-(Benzo[d]thiazol-7-yl)phenyl)-2-hydroxyethyl)-3-(2-ethynylthiazol-4-yl)urea A41. ¹H NMR (400 MHz, DMSO-d₆) δ 9.52 (s, 1H), 9.45 (s, 1H), 8.10 (dd, J=8.1, 1.0 Hz, 1H), 7.70 (t, J=5.3 Hz, 2H), 7.66 (d, J=8.0 Hz, 1H), 7.58 (d, J=6.8 Hz, 1H), 7.50 (d, J=8.2 Hz, 2H), 7.28 (s, 1H), 7.11 (d, J=7.8 Hz, 1H), 5.10 (t, J=5.1 Hz, 1H), 4.90 (s, 1H), 4.85 (dd, J=12.6, 5.2 Hz, 1H), 3.74 (dt, J=9.9, 4.9 Hz, 1H), 3.67 (dt, J=10.7, 5.4 Hz, 1H).

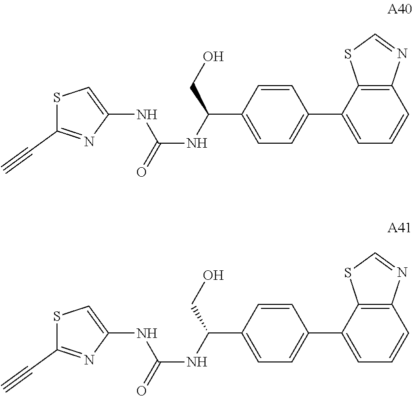 [SMILES: C#Cc1nc(NC(=O)N[C@@H](CO)c2ccc(-c3cccc4ncsc34)cc2)cs1 ; C#Cc1nc(NC(=O)N[C@H](CO)c2ccc(-c3cccc4ncsc34)cc2)cs1]

(R)-1-(2-Ethynylthiazol-4-yl)-3-(2-hydroxy-1-(3′-(pyrrolidin-1-yl)-[1,1′-biphenyl]-4-yl)ethyl)urea A42. ¹H NMR (400 MHz, DMSO-d₆) δ 9.48 (s, 1H), 7.57 (d, J=8.3 Hz, 2H), 7.36 (d, J=8.2 Hz, 2H), 7.26 (s, 1H), 7.22 (t, J=7.9 Hz, 1H), 7.03 (d, J=7.8 Hz, 1H), 6.82 (d, J=7.9 Hz, 1H), 6.71 (d, J=1.9 Hz, 1H), 6.52 (dd, J=8.1, 2.0 Hz, 1H), 5.03 (t, J=5.2 Hz, 1H), 4.89 (s, 1H), 4.78 (dd, J=12.8, 5.4 Hz, 1H), 3.69 (dt, J=10.0, 4.9 Hz, 1H), 3.61 (dt, J=10.9, 5.6 Hz, 1H), 3.28 (t, J=6.5 Hz, 4H), 1.99-1.92 (m, 4H).

(R)-2-(4-(2-(1-Cyanocyclopropyl)pyridin-3-yl)phenyl)-2-(3-(2-ethynylthiazol-4-yl)ureido)-N-methylacetamide A43. ¹H NMR (400 MHz, DMSO-d₆) δ 9.61 (s, 1H), 8.55 (dd, J=4.7, 1.7 Hz, 1H), 8.44 (d, J=4.6 Hz, 1H), 7.75 (dd, J=7.7, 1.7 Hz, 1H), 7.55-7.49 (m, 4H), 7.48 (d, J=2.8 Hz, 1H), 7.44 (d, J=8.1 Hz, 1H), 7.29 (s, 1H), 5.43 (d, J=8.1 Hz, 1H), 4.90 (s, 1H), 2.63 (d, J=4.6 Hz, 3H), 1.64 (dd, J=7.9, 4.8 Hz, 2H), 1.54 (dd, J=7.7, 4.6 Hz, 2H); MS (ESI) m/z: 457.1 [M+H⁺].

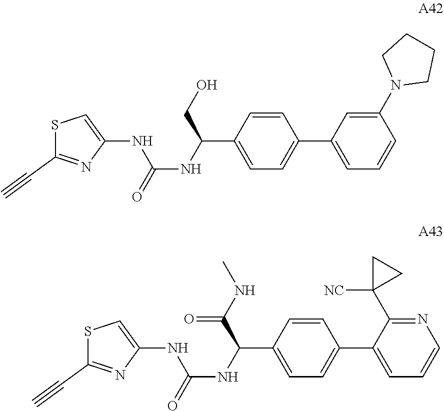 [SMILES: C#Cc1nc(NC(=O)N[C@@H](CO)c2ccc(-c3cccc(N4CCCC4)c3)cc2)cs1 ; C#Cc1nc(NC(=O)N[C@@H](C(=O)NC)c2ccc(-c3cccnc3C3(C#N)CC3)cc2)cs1]

(R)-1-(1-(2′-Cyano-[1,1′-biphenyl]-4-yl)-2-hydroxyethyl)-3-(2-ethynylthiazol-4-yl)urea A44. ¹H NMR (400 MHz, DMSO-d₆) δ 9.51 (s, 1H), 7.95 (dd, J=7.8, 1.0 Hz, 1H), 7.79 (td, J=7.7, 1.3 Hz, 1H), 7.62 (d, J=7.3 Hz, 1H), 7.58 (dd, J=11.1, 4.7 Hz, 3H), 7.47 (d, J=8.2 Hz, 2H), 7.28 (s, 1H), 7.09 (d, J=7.8 Hz, 1H), 5.10 (t, J=5.1 Hz, 1H), 4.90 (s, 1H), 4.85 (dd, J=5.0, 2.7 Hz, 1H), 3.70 (ddd, J=24.5, 10.8, 5.6 Hz, 2H); MS (ESI) m/z: 389.1 [M+H⁺].

(R)-1-(1-(5-(2-Cyanophenyl)pyridin-2-yl)-2-hydroxyethyl)-3-(2-ethynylthiazol-4-yl)urea A45. ¹H NMR (400 MHz, DMSO-d₆) δ 9.66 (s, 1H), 8.77 (dd, J=2.3, 0.6 Hz, 1H), 8.02 (td, J=8.3, 1.7 Hz, 2H), 7.84 (td, J=7.7, 1.3 Hz, 1H), 7.74-7.69 (m, 1H), 7.64 (td, J=7.7, 1.2 Hz, 1H), 7.52 (d, J=8.0 Hz, 1H), 7.29 (s, 1H), 7.19 (d, J=8.0 Hz, 1H), 5.06 (t, J=5.2 Hz, 1H), 4.97-4.91 (m, 1H), 4.90 (s, J=3.0 Hz, 1H), 3.82 (dt, J=10.2, 5.1 Hz, 1H), 3.74 (dt, J=10.6, 5.4 Hz 1H); MS (ESI) m/z: 390.0 [M+H⁺].

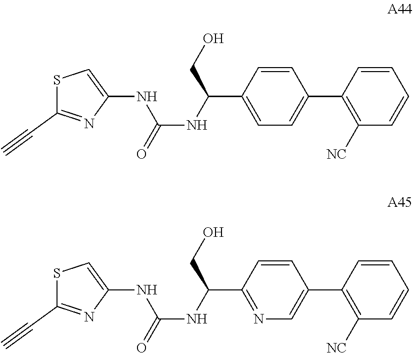 [SMILES: C#Cc1nc(NC(=O)N[C@@H](CO)c2ccc(-c3ccccc3C#N)cc2)cs1 ; C#Cc1nc(NC(=O)N[C@@H](CO)c2ccc(-c3ccccc3C#N)cn2)cs1]

(R)-2-(2′-Cyano-[1,1′-biphenyl]-4-yl)-2-(3-(2-ethynylthiazol-4-yl)ureido)ethyl carbamate A46. ¹H NMR (400 MHz, DMSO-d₆) δ 9.49 (s, 1H), 7.45 (d, J=8.3 Hz, 2H), 7.37 (d, J=8.3 Hz, 2H), 7.28 (d, J=5.1 Hz, 2H), 7.05 (d, J=8.2 Hz, 1H), 6.57 (s, 2H), 5.72 (t, J=4.5 Hz, 1H), 5.01 (dd, J=12.8, 7.7 Hz, 1H), 4.89 (s, 1H), 4.14 (ddd, J=18.8, 11.3, 6.3 Hz, 2H), 3.75 (s, 3H), 2.79 (t, J=8.7 Hz, 2H); MS (ESI) m/z: 432.1 [M+H⁺].

(R)-2-(4-(3-Cyanopyridin-2-yl)phenyl)-2-(3-(2-ethynylthiazol-4-yl)ureido)ethyl carbamate A47. ¹H NMR (400 MHz, DMSO-d₆) δ 9.54 (s, 1H), 8.94 (dd, J=4.8, 1.7 Hz, 1H), 8.43 (dd, J=7.9, 1.7 Hz, 1H), 7.88 (d, J=8.3 Hz, 2H), 7.61 (dd, J=7.9, 4.8 Hz, 1H), 7.55 (d, J=8.3 Hz, 2H), 7.29 (s, 1H), 7.11 (d, J=8.1 Hz, 1H), 6.65 (s, 2H), 5.10 (dd, J=12.5, 7.4 Hz, 1H), 4.89 (s, 1H), 4.21 (ddd, J=18.4, 11.3, 6.1 Hz, 2H); MS (ESI) m/z: 433.1 [M+H⁺].

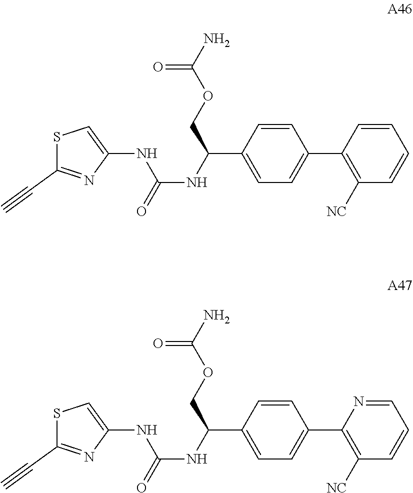 [SMILES: C#Cc1nc(NC(=O)N[C@@H](COC(N)=O)c2ccc(-c3ccccc3C#N)cc2)cs1 ; C#Cc1nc(NC(=O)N[C@@H](COC(N)=O)c2ccc(-c3ncccc3C#N)cc2)cs1]

(R)-2-(4-(4-Cyanopyridin-2-yl)phenyl)-2-(3-(2-ethynylthiazol-4-yl)ureido)ethyl carbamate A48. ¹H NMR (400 MHz, DMSO-d₆) δ 9.51 (s, 1H), 8.90 (dt, J=2.9, 1.4 Hz, 1H), 8.47 (d, J=8.2 Hz, 1H), 8.14 (dd, J=12.9, 8.4 Hz, 2H), 7.80 (td, J=5.2, 1.4 Hz, 1H), 7.48 (dd, J=20.1, 8.4 Hz, 2H), 7.27 (d, J=8.1 Hz, 1H), 7.08 (d, J=8.2 Hz, 1H), 6.57 (s, 2H), 5.07 (dd, J=12.2, 7.9 Hz, 1H), 4.90 (s, 1H), 4.18 (qd, J=11.3, 6.1 Hz, 2H); MS (ESI) m/z: 433.1 [M+H⁺].

(R)-2-(3-(2-Ethynylthiazol-4-yl)ureido)-2-(4-(isoquinolin-8-yl)phenyl)ethyl carbamate A49. ¹H NMR (400 MHz, DMSO-d₆) δ 9.53 (s, 1H), 8.30 (t, J=7.3 Hz, 1H), 8.16-8.04 (m, 3H), 7.98 (dd, J=7.2, 4.7 Hz, 1H), 7.50 (dd, J=20.1, 8.3 Hz, 2H), 7.27 (d, J=7.9 Hz, 1H), 7.12 (s, 1H), 6.57 (s, 1H), 5.14-5.01 (m, 1H), 4.90-4.80 (m, 1H), 4.27-4.05 (m, 1H), 3.66 (ddd, J=21.1, 10.5, 5.5 Hz, 1H); MS (ESI) m/z: 433.1 [M+H⁺].

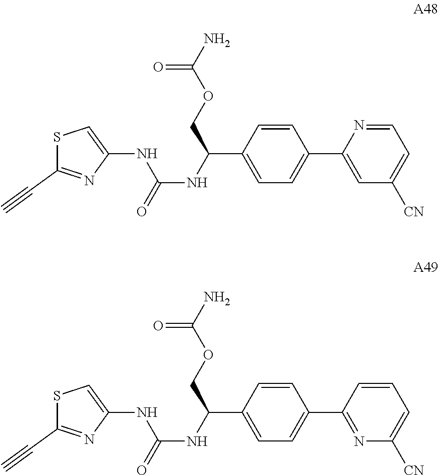 [SMILES: C#Cc1nc(NC(=O)N[C@@H](COC(N)=O)c2ccc(-c3cc(C#N)ccn3)cc2)cs1 ; C#Cc1nc(NC(=O)N[C@@H](COC(N)=O)c2ccc(-c3cccc(C#N)n3)cc2)cs1]

(R)-2-(3-(2-Ethynylthiazol-4-yl)ureido)-2-(4-(quinazolin-8-yl)phenyl)ethyl carbamate A50. ¹H NMR (400 MHz, DMSO-d₆) δ 9.67 (s, 1H), 9.54 (s, 1H), 9.30 (s, 1H), 8.18 (d, J=8.1 Hz, 1H), 8.07 (dd, J=7.2, 1.3 Hz, 1H), 7.86 (t, J=7.7 Hz, 1H), 7.70 (d, J=8.2 Hz, 2H), 7.49 (d, J=8.2 Hz, 2H), 7.30 (s, 1H), 7.12 (s, 1H), 6.59 (s, 2H), 5.08 (dd, J=12.9, 7.5 Hz, 1H), 4.22 (ddd, J=18.5, 11.2, 6.2 Hz, 2H); MS (ESI) m/z: 459.0 [M+H⁺].

(R)-2-(3-(2-Ethynylthiazol-4-yl)ureido)-2-(4-(quinoxalin-5-yl)phenyl)ethyl carbamate A51. ¹H NMR (400 MHz, DMSO-d₆) δ 9.53 (s, 1H), 8.98 (s, 1H), 8.95 (d, J=1.6 Hz, 1H), 8.12 (dd, J=8.2, 1.3 Hz, 1H), 7.94 (t, J=7.7 Hz, 1H), 7.91-7.86 (m, 1H), 7.65 (d, J=8.2 Hz, 2H), 7.48 (d, J=8.2 Hz, 2H), 7.30 (s, 1H), 7.10 (d, J=8.2 Hz, 1H), 6.58 (s, 2H), 5.08 (dd, J=12.7, 7.4 Hz, 1H), 4.90 (s, 1H), 4.23 (ddd, J=18.6, 11.2, 6.2 Hz, 2H); MS (ESI) m/z: 459.1 [M+H⁺].

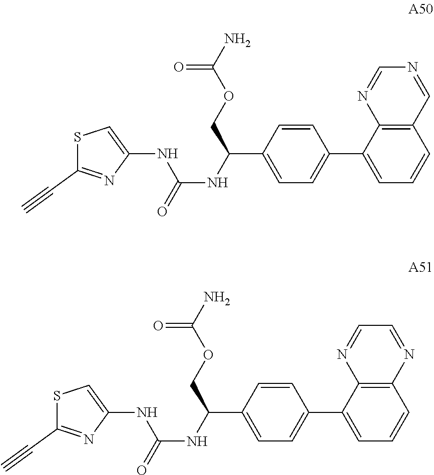 [SMILES: C#Cc1nc(NC(=O)N[C@@H](COC(N)=O)c2ccc(-c3cccc4cncnc34)cc2)cs1 ; C#Cc1nc(NC(=O)N[C@@H](COC(N)=O)c2ccc(-c3cccc4nccnc34)cc2)cs1]

(R)-1-(1-(4-(7-Cyanoquinolin-8-yl)phenyl)-2-hydroxyethyl)-3-(2-ethynylthiazol-4-yl)urea A52. ¹H NMR (400 MHz, DMSO-d₆) δ 9.54 (s, 1H), 9.00 (dd, J=4.1, 1.6 Hz, 1H), 8.55 (dd, J=8.3, 1.5 Hz, 1H), 8.20 (d, J=8.5 Hz, 1H), 8.00 (d, J=8.5 Hz, 1H), 7.72 (dd, J=8.3, 4.1 Hz, 1H), 7.50 (q, J=8.3 Hz, 4H), 7.31 (s, 1H), 7.12 (d, J=8.0 Hz, 1H), 5.16 (t, J=4.9 Hz, 1H), 4.96-4.79 (m, 2H), 3.75 (dtd, J=21.4, 10.6, 5.1 Hz, 2H); MS (ESI) m/z: 440.0 [M+H⁺].

(R)-1-(1-(4-(3-Cyano-1-methyl-1H-indazol-4-yl)phenyl)-2-hydroxyethyl)-3-(2-ethynylthiazol-4-yl)urea A53. ¹H NMR (400 MHz, DMSO-d₆) δ 9.51 (s, 1H), 7.89 (d, J=8.5 Hz, 1H), 7.68-7.62 (m, 1H), 7.59 (d, J=8.1 Hz, 2H), 7.47 (d, J=8.1 Hz, 2H), 7.35 (d, J=7.1 Hz, 1H), 7.29 (s, 1H), 7.09 (d, J=8.0 Hz, 1H), 5.10 (s, 1H), 4.90 (s, 2H), 3.95-3.56 (m, 2H); MS (ESI) m/z: 443.2 [M+H⁺].

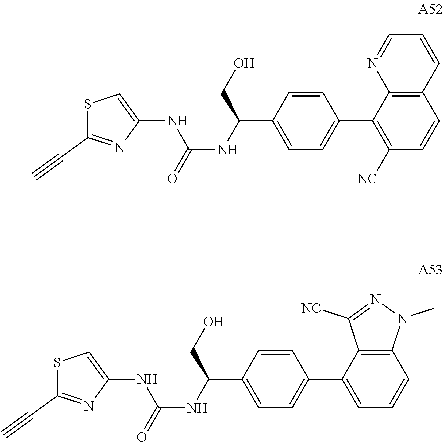 [SMILES: C#Cc1nc(NC(=O)N[C@@H](CO)c2ccc(-c3c(C#N)ccc4cccnc34)cc2)cs1 ; C#Cc1nc(NC(=O)N[C@@H](CO)c2ccc(-c3cccc4c3c(C#N)nn4C)cc2)cs1]

(R)-1-(1-(2′-(1-Cyanocyclopropyl)-[1,1′-biphenyl]-4-yl)-2-hydroxyethyl)-3-(2-ethynylthiazol-4-yl)urea A54. ¹H NMR (400 MHz, DMSO-d₆) δ 9.50 (s, 1H), 7.56 (dd, J=7.4, 1.6 Hz, 1H), 7.47-7.38 (m, 6H), 7.30-7.25 (m, 2H), 7.07 (d, J=7.5 Hz, 1H), 5.06 (t, J=5.1 Hz, 1H), 4.88 (d, J=10.3 Hz, 2H), 3.69 (dd, J=21.7, 5.2 Hz, 2H), 1.41 (dd, J=7.4, 4.8 Hz, 2H), 1.10 (q, J=5.0 Hz, 2H); MS (ESI) m/z: 429.1 [M+H⁺].

(R)-2-(2′-(1-Cyanocyclopropyl)-[1,1′-biphenyl]-4-yl)-2-(3-(2-ethynylthiazol-4-yl)ureido)ethyl carbamate A55. ¹H NMR (400 MHz, DMSO-d₆) δ 9.52 (s, 1H), 7.57 (dd, J=7.3, 1.6 Hz, 1H), 7.52-7.39 (m, 6H), 7.35-7.24 (m, 2H), 7.10 (d, J=8.4 Hz, 1H), 6.59 (s, 2H), 5.12 (dd, J=12.7, 7.7 Hz, 1H), 4.89 (s, 1H), 4.20 (ddd, J=18.8, 11.2, 6.2 Hz, 2H), 1.42 (dd, J=7.2, 4.8 Hz, 2H), 1.10 (q, J=5.1 Hz, 2H); MS (ESI) m/z: 472.2 [M+H⁺].

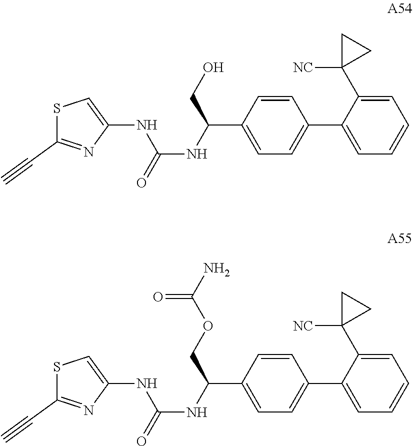 [SMILES: C#Cc1nc(NC(=O)N[C@@H](CO)c2ccc(-c3ccccc3C3(C#N)CC3)cc2)cs1 ; C#Cc1nc(NC(=O)N[C@@H](COC(N)=O)c2ccc(-c3ccccc3C3(C#N)CC3)cc2)cs1]

(R)-2-(4-(6-(1-Cyanocyclopropyl)pyridin-2-yl)phenyl)-2-(3-(2-ethynylthiazol-4-yl)ureido)ethyl carbamate A56. ¹H NMR (400 MHz, DMSO-d₆) δ 9.51 (s, 1H), 8.03 (t, J=7.2 Hz, 2H), 7.92 (dt, J=7.7, 3.5 Hz, 1H), 7.89-7.82 (m, 1H), 7.54-7.49 (m, 1H), 7.47 (d, J=8.3 Hz, 2H), 7.29-7.25 (m, 1H), 7.07 (d, J=8.1 Hz, 1H), 6.58 (s, 2H), 5.04 (dd, J=12.6, 7.4 Hz, 1H), 4.90 (s, 1H), 4.28-4.07 (m, 2H), 1.93-1.75 (m, 4H); MS (ESI) m/z: 473.1 [M+H⁺].

(R)-2-(4-(4-(1-Cyanocyclopropyl)pyridin-2-yl)phenyl)-2-(3-(2-ethynylthiazol-4-yl)ureido)ethyl carbamate A57. ¹H NMR (400 MHz, DMSO-d₆) δ 9.50 (s, 1H), 8.63 (d, J=5.2 Hz, 1H), 8.06 (t, J=11.1 Hz, 2H), 7.65 (d, J=1.2 Hz, 1H), 7.47 (t, J=9.8 Hz, 2H), 7.34 (dd, J=5.2, 1.8 Hz, 1H), 7.27 (d, J=8.0 Hz, 1H), 7.07 (d, J=8.1 Hz, 1H), 6.57 (s, 2H), 5.06 (dd, J=12.7, 7.4 Hz, 1H), 4.90 (s, 1H), 4.17 (qd, J=11.3, 6.2 Hz, 2H), 1.92 (dd, J=8.0, 4.9 Hz, 2H), 1.83-1.74 (m, 2H); MS (ESI) m/z: 473.1 [M+H⁺].

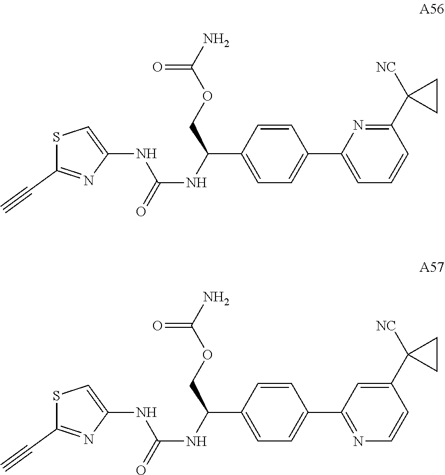 [SMILES: C#Cc1nc(NC(=O)N[C@@H](COC(N)=O)c2ccc(-c3cccc(C4(C#N)CC4)n3)cc2)cs1 ; C#Cc1nc(NC(=O)N[C@@H](COC(N)=O)c2ccc(-c3cc(C4(C#N)CC4)ccn3)cc2)cs1]

(R)-2-(4-(3-(1-Cyanocyclopropyl)pyridin-2-yl)phenyl)-2-(3-(2-ethynylthiazol-4-yl)ureido)ethyl carbamate A58. ¹H NMR (400 MHz, DMSO-d₆) δ 9.56 (s, 1H), 8.66 (dd, J=4.7, 1.5 Hz, 1H), 8.02 (dd, J=7.8, 1.5 Hz, 1H), 7.63 (d, J=8.2 Hz, 2H), 7.52 (d, J=8.2 Hz, 2H), 7.45 (dd, J=7.8, 4.7 Hz, 1H), 7.31 (s, 1H), 7.15 (d, J=8.4 Hz, 1H), 6.58 (s, 2H), 5.14 (dd, J=12.6, 7.6 Hz, 1H), 4.90 (s, 1H), 4.21 (ddd, J=18.6, 11.2, 6.2 Hz, 2H), 1.54 (q, J=4.8 Hz, 2H), 1.19 (dd, J=7.1, 4.9 Hz, 2H); MS (ESI) m/z: 473.2 [M+H⁺].

(R)-1-(1-(6-(2-(1-Cyanocyclopropyl)phenyl)pyridin-3-yl)-2-hydroxyethyl)-3-(2-ethynylthiazol-4-yl)urea A59. ¹H NMR (400 MHz, DMSO-d₆) δ 9.67 (s, 1H), 8.63 (d, J=2.1 Hz, 1H), 7.85 (dd, J=8.0, 2.3 Hz, 1H), 7.59 (dd, J=7.2, 1.8 Hz, 1H), 7.49 (ddd, J=10.5, 5.9, 4.2 Hz, 3H), 7.35 (dd, J=7.0, 1.9 Hz, 1H), 7.31 (s, 1H), 7.21 (d, J=8.2 Hz, 1H), 5.01 (s, 1H), 4.98-4.92 (m, 1H), 4.90 (s, 1H), 3.77 (td, J=10.4, 5.4 Hz, 2H), 1.45 (q, J=4.8 Hz, 2H), 1.21 (dd, J=7.6, 5.1 Hz, 2H); MS (ESI) m/z: 430.1 [M+H⁺].

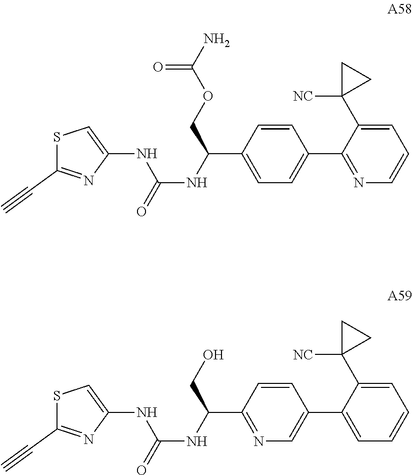 [SMILES: C#Cc1nc(NC(=O)N[C@@H](COC(N)=O)c2ccc(-c3ncccc3C3(C#N)CC3)cc2)cs1 ; C#Cc1nc(NC(=O)N[C@@H](CO)c2ccc(-c3ccccc3C3(C#N)CC3)cn2)cs1]

(R)-2-(5-(2-(1-Cyanocyclopropyl)phenyl)pyridin-2-yl)-2-(3-(2-ethynylthiazol-4-yl)ureido)ethyl carbamate A60. ¹H NMR (400 MHz, DMSO-d₆) δ 9.68 (s, 1H), 8.65 (d, J=1.7 Hz, 1H), 7.89 (dd, J=8.0, 2.3 Hz, 1H), 7.62-7.58 (m, 1H), 7.56 (d, J=8.0 Hz, 1H), 7.53-7.45 (m, 2H), 7.38-7.34 (m, 1H), 7.33 (s, 1H), 7.23 (d, J=8.3 Hz, 1H), 6.52 (s, 2H), 5.21 (dd, J=14.2, 6.2 Hz, 1H), 4.90 (s, 1H), 4.35-4.20 (m, 2H), 1.46 (dd, J=7.3, 4.7 Hz, 2H), 1.20 (dd, J=7.5, 5.0 Hz, 2H); MS (ESI) m/z: 473.2 [M+H⁺].

(S)-2-(5-(2-(1-Cyanocyclopropyl)phenyl)pyridin-2-yl)-2-(3-(2-ethynylthiazol-4-yl)ureido)ethyl carbamate A61. ¹H NMR (400 MHz, DMSO-d₆) δ 9.67 (s, 1H), 8.65 (d, J=2.2 Hz, 1H), 7.89 (dd, J=8.0, 2.3 Hz, 1H), 7.62-7.58 (m, 1H), 7.56 (d, J=8.0 Hz, 1H), 7.53-7.45 (m, 2H), 7.38-7.34 (m, 1H), 7.33 (s, 1H), 7.23 (d, J=8.3 Hz, 1H), 6.53 (s, 2H), 5.20 (dd, J=14.3, 6.2 Hz, 1H), 4.90 (s, 1H), 4.36-4.20 (m, 2H), 1.46 (dd, J=7.3, 4.7 Hz, 2H), 1.20 (dd, J=7.5, 5.0 Hz, 2H); MS (ESI) m/z: 473.2 [M+H⁺].

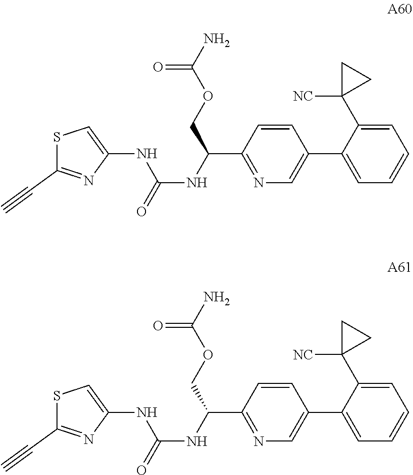 [SMILES: C#Cc1nc(NC(=O)N[C@@H](COC(N)=O)c2ccc(-c3ccccc3C3(C#N)CC3)cn2)cs1 ; C#Cc1nc(NC(=O)N[C@H](COC(N)=O)c2ccc(-c3ccccc3C3(C#N)CC3)cn2)cs1]

(R)-1-(1-(4-(2-(1-Cyanocyclopropyl)pyridin-3-yl)phenyl)-2-hydroxyethyl)-3-(2-ethynylthiazol-4-yl)urea A62. ¹H NMR (400 MHz, DMSO-d₆) δ 9.50 (s, 1H), 8.55 (dd, J=4.7, 1.7 Hz, 1H), 7.74 (dd, J=7.7, 1.7 Hz, 1H), 7.51-7.47 (m, 1H), 7.47 (s, 4H), 7.29 (d, J=4.7 Hz, 1H), 7.06 (d, J=8.1 Hz, 1H), 5.05 (t, J=5.1 Hz, 1H), 4.93-4.85 (m, 2H), 3.68 (dtd, J=21.8, 10.8, 5.2 Hz, 2H), 1.64-1.51 (m, 4H); MS (ESI) m/z: 430.1 [M+H⁺].

(R)-2-(4-(2-(1-Cyanocyclopropyl)pyridin-3-yl)phenyl)-2-(3-(2-ethynylthiazol-4-yl)ureido)ethyl carbamate A63. ¹H NMR (400 MHz, DMSO-d₆) δ 9.51 (s, 1H), 8.55 (dd, J=4.7, 1.7 Hz, 1H), 7.76 (dd, J=7.7, 1.7 Hz, 1H), 7.56-7.44 (m, 5H), 7.30 (s, 1H), 7.08 (d, J=8.4 Hz, 1H), 6.59 (s, 2H), 5.12 (td, J=8.0, 4.7 Hz, 1H), 4.90 (s, 1H), 4.22 (dd, J=11.3, 4.7 Hz, 1H), 4.13 (dd, J=11.2, 7.8 Hz, 1H), 1.64-1.58 (m, 2H), 1.54 (dt, J=4.7, 3.3 Hz, 2H); MS (ESI) m/z: 473.2 [M+H⁺].

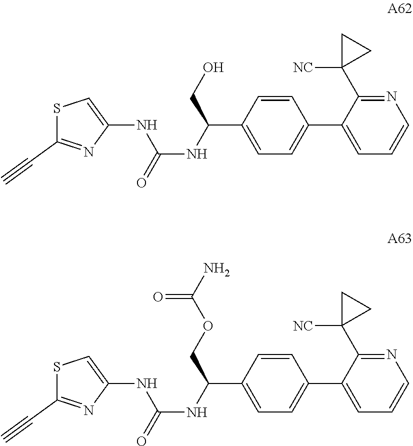 [SMILES: C#Cc1nc(NC(=O)N[C@@H](CO)c2ccc(-c3cccnc3C3(C#N)CC3)cc2)cs1 ; C#Cc1nc(NC(=O)N[C@@H](COC(N)=O)c2ccc(-c3cccnc3C3(C#N)CC3)cc2)cs1]

(R)-1-(1-(4-(2-(1-Cyanocyclopropyl)-5-fluoropyridin-3-yl)phenyl)-2-hydroxy-ethyl)-3-(2-ethynylthiazol-4-yl)urea A64. ¹H NMR (400 MHz, DMSO-d₆) δ 9.51 (s, 1H), 8.56 (d, J=2.8 Hz, 1H), 7.75 (dd, J=9.1, 2.8 Hz, 1H), 7.65-7.40 (m, 4H), 7.28 (s, 1H), 7.10-6.93 (m, 1H), 5.07 (t, J=4.8 Hz, 1H), 4.90 (s, 2H), 3.94-3.48 (m, 2H), 2.07 (s, 1H), 1.55 (tt, J=7.5, 3.7 Hz, 4H); MS (ESI) m/z: 448.2 [M+H⁺].

(R)-1-(1-(3-Chloro-4-(2-(1-cyanocyclopropyl)pyridin-3-yl)phenyl)-2-hydroxy-ethyl)-3-(2-ethynylthiazol-4-yl)urea A65. ¹H NMR (400 MHz, DMSO-d₆) δ 9.52 (d, J=3.4 Hz, 1H), 8.58 (dd, J=4.7, 1.7 Hz, 1H), 7.68 (dd, J=7.7, 1.7 Hz, 1H), 7.56 (d, J=4.2 Hz, 1H), 7.51-7.44 (m, 2H), 7.43 (d, J=2.1 Hz, 1H), 7.30 (d, 1=0.7 Hz, 1H), 7.13 (d, J=8.1 Hz, 1H), 5.10 (s, 1H), 4.98-4.83 (m, 2H), 3.90-3.44 (m, 2H), 1.85-1.68 (m, 1H), 1.67-1.58 (m, 1H), 1.57-1.43 (m, 2H); MS (ESI) m/z: 464.1 [M+H⁺].

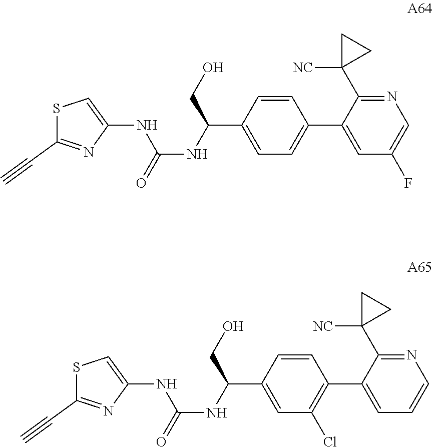 [SMILES: C#Cc1nc(NC(=O)N[C@@H](CO)c2ccc(-c3cc(F)cnc3C3(C#N)CC3)cc2)cs1 ; C#Cc1nc(NC(=O)N[C@@H](CO)c2ccc(-c3cccnc3C3(C#N)CC3)c(Cl)c2)cs1]

(S)-1-(1-(3-Chloro-4-(2-(1-cyanocyclopropyl)pyridin-3-yl)phenyl)-2-hydroxy-ethyl)-3-(2-ethynylthiazol-4-yl)urea A66. ¹H NMR (400 MHz, DMSO-d₆) δ 9.52 (d, J=2.9 Hz, 1H), 8.58 (d, J=3.4 Hz, 1H), 7.69 (d, J=6.7 Hz, 1H), 7.56 (d, J=4.6 Hz, 1H), 7.52-7.39 (m, 3H), 7.30 (s, 1H), 7.12 (d, J=8.0 Hz, 1H), 5.10 (s, 1H), 4.90 (s, 2H), 3.70 (dd, J=11.4, 6.7 Hz, 2H), 1.87-1.69 (m, 1H), 1.68-1.58 (m, 1H), 1.51 (s, 2H); MS (ESI) m/z: 464.1 [M+H⁺].

(R)-1-(1-(3-Chloro-4-(2-(1-cyanocyclopropyl)pyridin-3-yl)phenyl)-2-hydroxy-ethyl)-3-(2-ethynylthiazol-4-yl)-1-methylurea A67. ¹H NMR (400 MHz, DMSO-d₆) δ 9.63 (s, 1H), 8.57 (dd, J=4.7, 1.7 Hz, 1H), 7.71 (dd, J=7.7, 1.7 Hz, 1H), 7.54-7.35 (m, 5H), 5.53 (d, J=7.3 Hz, 1H), 5.08 (t, J=5.2 Hz, 1H), 4.90 (s, 1H), 4.03-3.85 (m, 2H), 2.80 (s, 3H); MS (ESI) m/z: 478.1 [M+H⁺].

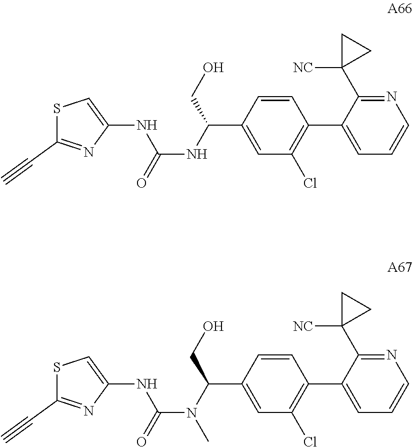 [SMILES: C#Cc1nc(NC(=O)N[C@H](CO)c2ccc(-c3cccnc3C3(C#N)CC3)c(Cl)c2)cs1 ; C#Cc1nc(NC(=O)N(C)[C@@H](CO)c2ccc(-c3cccnc3C3(C#N)CC3)c(Cl)c2)cs1]

(R)-1-(1-(4-(2-(1-Cyanocyclopropyl)pyridin-3-yl)-3-fluorophenyl)-2-hydroxy-ethyl)-3-(2-ethynylthiazol-4-yl)urea A68. ¹H NMR (400 MHz, DMSO-d₆) δ 9.52 (s, 1H), 8.58 (dd, J=4.8, 1.7 Hz, 1H), 7.76 (dd, J=7.7, 1.5 Hz, 1H), 7.49 (dd, J=7.7, 4.8 Hz, 1H), 7.44 (t, J=7.8 Hz, 1H), 7.30 (t, J=4.7 Hz, 3H), 7.10 (d, J=8.1 Hz, 1H), 5.09 (t, J=5.1 Hz, 1H), 4.96-4.83 (m, 2H), 3.78-3.59 (m, 2H), 1.64 (dd, J=4.7, 3.2 Hz, 2H), 1.56-1.46 (m, 2H); MS (ESI) m/z: 448.1 [M+H⁺].

(R)-2-(3-Chloro-4-(2-(1-cyanocyclopropyl)pyridin-3-yl)phenyl)-2-(3-(2-ethynyl-thiazol-4-yl)ureido)ethyl carbamate A69. ¹H NMR (400 MHz, DMSO-d₆) δ 9.55 (d, J=2.9 Hz, 1H), 8.58 (dd, J=4.7, 1.7 Hz, 1H), 7.72-7.68 (m, 1H), 7.61 (d, J=2.1 Hz, 1H), 7.51-7.44 (m, 3H), 7.30 (t, J=2.4 Hz, 1H), 7.12 (d, J=8.2 Hz, 1H), 6.61 (s, 2H), 5.13 (td, J=7.8, 4.7 Hz, 1H), 4.90 (d, J=0.7 Hz, 1H), 4.26-4.10 (m, 2H), 1.69 (dddd, J=12.8, 7.3, 4.6, 1.7 Hz, 2H), 1.51 (ddd, J=9.7, 7.5, 3.3 Hz, 2H); MS (ESI) m/z: 507.1 [M+H⁺].

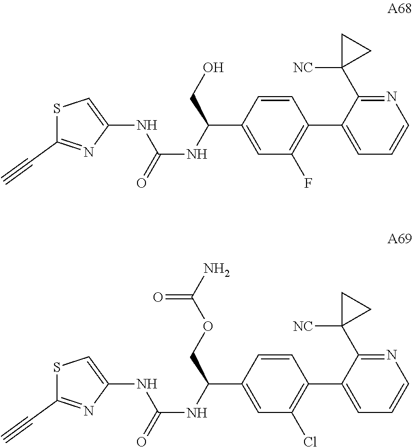 [SMILES: C#Cc1nc(NC(=O)N[C@@H](CO)c2ccc(-c3cccnc3C3(C#N)CC3)c(F)c2)cs1 ; C#Cc1nc(NC(=O)N[C@@H](COC(N)=O)c2ccc(-c3cccnc3C3(C#N)CC3)c(Cl)c2)cs1]

(R)-2-(3-Chloro-4-(2-(1-cyanocyclopropyl)pyridin-3-yl)phenyl)-2-(3-(2-ethynyl-thiazol-4-yl)-1-methylureido)ethyl carbamate A70. ¹H NMR (400 MHz, DMSO-d₆) δ 9.72 (s, 1H), 8.65-8.50 (m, 1H), 7.81-7.65 (m, 1H), 7.59-7.44 (m, 4H), 7.41 (d, J=5.3 Hz, 1H), 6.66 (s, 2H), 5.93-5.71 (m, 1H), 4.90 (d, J=1.3 Hz, 1H), 4.67-4.34 (m, 2H), 2.83 (s, 3H), 1.79 (dd, J=9.7, 5.9 Hz, 1H), 1.67 (d, J=6.1 Hz, 1H), 1.55 (d, J=15.8 Hz, 2H); MS (ESI) m/z: 521.0 [M+H⁺].

(R)-1-(1-(3-Chloro-4-(2-(1-cyanocyclopropyl)pyridin-3-yl)phenyl)-2-hydroxy-ethyl)-3-(5-ethynyl-1,3,4-thiadiazol-2-yl)urea A71. ¹H NMR (400 MHz, DMSO-d₆) δ 11.27 (s, 1H), 8.58 (dd, J=4.7, 1.7 Hz, 1H), 7.69 (dd, J=7.7, 1.7 Hz, 1H), 7.59 (d, J=4.2 Hz, 1H), 7.53-7.40 (m, 4H), 5.18 (s, 1H), 5.04 (s, 1H), 4.93 (d, J=7.6 Hz, 1H), 3.81-3.57 (m, 2H), 1.81-1.68 (m, 1H), 1.69-1.59 (m, 1H), 1.57-1.44 (m, 2H); MS (ESI) m/z: 465.0 [M+H⁺].

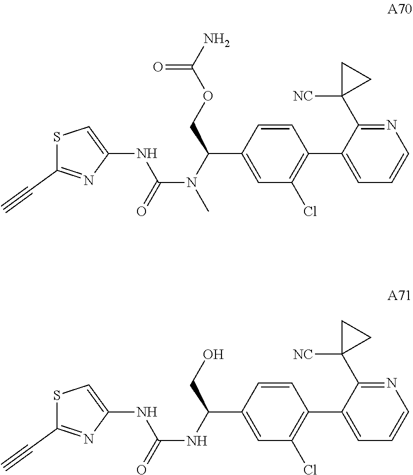 [SMILES: C#Cc1nc(NC(=O)N(C)[C@@H](COC(N)=O)c2ccc(-c3cccnc3C3(C#N)CC3)c(Cl)c2)cs1 ; C#Cc1nc(NC(=O)N[C@@H](CO)c2ccc(-c3cccnc3C3(C#N)CC3)c(Cl)c2)cs1]

(R)-1-(1-(3-Chloro-4-(2-(1-cyanocyclopropyl)pyridin-3-yl)phenyl)-2-(methyl-sulfonyl)ethyl)-3-(2-ethynylthiazol-4-yl)urea A72. ¹H NMR (400 MHz, DMSO-d₆) δ 9.70 (s, 1H), 8.58 (dd, J=4.7, 1.7 Hz, 1H), 7.75-7.64 (m, 2H), 7.56-7.43 (m, 3H), 7.33 (s, 1H), 7.21 (dd, J=8.6, 3.1 Hz, 1H), 5.45 (td, J=8.8, 4.6 Hz, 1H), 4.90 (d, J=1.0 Hz, 1H), 3.85 (dd, J=14.6, 9.0 Hz, 1H), 3.70 (dd, J=14.6, 4.6 Hz, 1H), 2.96 (d, J=3.2 Hz, 3H), 1.82-1.58 (m, 2H), 1.57-1.42 (m, 2H); MS (ESI) m/z: 526.0 [M+H⁺].

(R)-2-(3-Chloro-4-(2-(1-cyanocyclopropyl)pyridin-3-yl)phenyl)-2-(3-(2-ethynyl-thiazol-4-yl)ureido)ethanesulfonamide A73. ¹H NMR (400 MHz, DMSO-d₆) δ 9.68 (d, J=3.1 Hz, 1H), 8.58 (dd, J=4.7, 1.7 Hz, 1H), 7.74-7.67 (m, 1H), 7.64 (s, 1H), 7.55-7.41 (m, 3H), 7.31 (s, 1H), 7.15 (d, J=7.8 Hz, 1H), 6.94 (s, 2H), 5.37 (s, 1H), 4.90 (d, J=0.7 Hz, 1H), 3.66 (ddd, J=14.5, 8.8, 5.8 Hz, 1H), 3.46 (dt, J=14.4, 4.1 Hz, 1H), 1.80-1.69 (m, 1H), 1.69-1.58 (m, 1H), 1.59-1.40 (m, 2H); MS (ESI) m/z: 527.1 [M+H⁺].

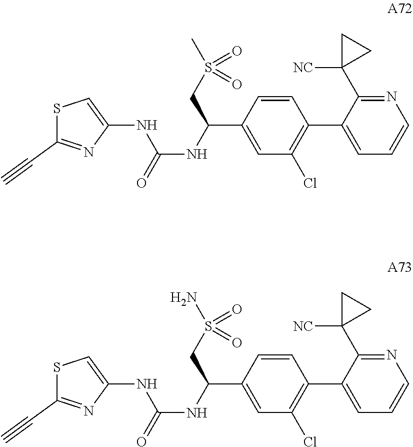 [SMILES: C#Cc1nc(NC(=O)N[C@@H](CS(C)(=O)=O)c2ccc(-c3cccnc3C3(C#N)CC3)c(Cl)c2)cs1 ; C#Cc1nc(NC(=O)N[C@@H](CS(N)(=O)=O)c2ccc(-c3cccnc3C3(C#N)CC3)c(Cl)c2)cs1]

(R)-2-(3-Chloro-4-(2-(1-cyanocyclopropyl)pyridin-3-yl)phenyl)-2-(3-(2-ethynyl-thiazol-4-yl)ureido)-N-methylacetamide A74. ¹H NMR (400 MHz, DMSO-d₆) δ 9.61 (s, 1H), 8.58 (dd, J=4.7, 1.7 Hz, 1H), 8.48 (dd, J=4.5, 2.4 Hz, 1H), 7.75-7.66 (m, 1H), 7.65-7.57 (m, 1H), 7.54-7.41 (m, 4H), 7.29 (s, 1H), 5.44 (dd, J=8.0, 2.7 Hz, 1H), 4.91 (s, 1H), 2.63 (dd, J=4.5, 2.6 Hz, 3H), 1.81-1.71 (m, 1H), 1.68-1.58 (m, 1H), 1.58-1.37 (m, 2H); MS (ESI) m/z: 491.1 [M+H⁺].

(R)-1-(1-(5-(2-(1-Cyanocyclopropyl)-4-fluorophenyl)pyridin-2-yl)-2-hydroxy-ethyl)-3-(2-ethynylthiazol-4-yl)urea A75. ¹H NMR (400 MHz, DMSO-d₆) δ 9.66 (s, 1H), 8.61 (dd, J=2.3, 0.6 Hz, 1H), 7.84 (dd, J=8.0, 2.3 Hz, 1H), 7.53-7.45 (m, 2H), 7.43-7.32 (m, 2H), 7.31 (s, 1H), 7.20 (d, J=8.2 Hz, 1H), 5.01 (t, J=5.3 Hz, 1H), 4.96 (dt, J=8.1, 5.4 Hz, 1H), 4.90 (s, 1H), 3.76 (ddt, J=35.3, 10.7, 5.3 Hz, 2H), 1.46 (dd, J=7.6, 4.9 Hz, 2H), 1.32 (dd, J=7.7, 5.2 Hz, 2H); MS (ESI) m/z: 448.1 [M+H⁺].

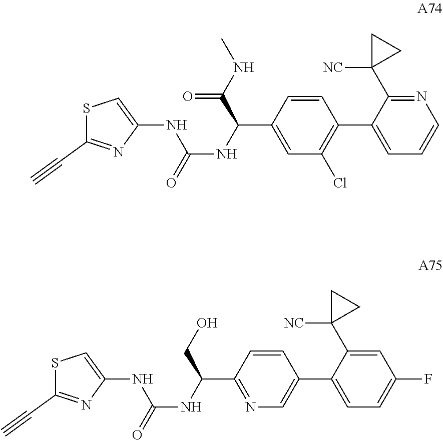 [SMILES: C#Cc1nc(NC(=O)N[C@@H](C(=O)NC)c2ccc(-c3cccnc3C3(C#N)CC3)c(Cl)c2)cs1 ; C#Cc1nc(NC(=O)N[C@@H](CO)c2ccc(-c3ccc(F)cc3C3(C#N)CC3)cn2)cs1]

(R)-2-(5-(2-(1-Cyanocyclopropyl)-4-fluorophenyl)pyridin-2-yl)-2-(3-(2-ethynyl-thiazol-4-yl)ureido)ethyl carbamate A76. ¹H NMR (400 MHz, DMSO-d₆) δ 9.66 (s, 1H), 8.63 (d, J=1.7 Hz, 1H), 7.88 (dd, J=8.0, 2.3 Hz, 1H), 7.55 (d, J=8.0 Hz, 1H), 7.50 (dd, J=9.8, 2.6 Hz, 1H), 7.44-7.33 (m, 2H), 7.32 (s, 1H), 7.21 (d, J=8.4 Hz, 1H), 6.52 (s, 2H), 5.20 (dd, J=14.3, 6.1 Hz, 1H), 4.90 (s, 1H), 4.38-4.18 (m, 2H), 1.46 (dd, J=7.5, 4.8 Hz, 2H), 1.31 (dd, J=7.7, 5.1 Hz, 2H); MS (ESI) m/z: 491.1 [M+H⁺].

(R)-1-(1-(5-(2-(1-Cyanocyclopropyl)-5-fluorophenyl)pyridin-2-yl)-2-hydroxyethyl)-3-(2-ethynylthiazol-4-yl)urea A77. ¹H NMR (400 MHz, DMSO-d₆) δ 9.66 (s, 1H), 8.72-8.52 (m, 1H), 7.88 (dd, J=8.0, 2.3 Hz, 1H), 7.65 (dd, J=8.6, 5.8 Hz, 1H), 7.51 (d, J=8.0 Hz, 1H), 7.36-7.28 (m, 2H), 7.25 (dd, J=9.4, 2.8 Hz, 1H), 7.21 (d, J=8.2 Hz, 1H), 5.02 (t, J=5.2 Hz, 1H), 5.00-4.92 (m, 1H), 4.90 (s, 1H), 3.81 (dt, J=10.1, 5.0 Hz, 1H), 3.72 (dt, J=10.6, 5.4 Hz, 1H), 1.44 (dd, J=7.5, 4.8 Hz, 2H), 1.20 (dd, J=7.6, 5.1 Hz, 2H); MS (ESI) m/z: 448.2 [M+H⁺].

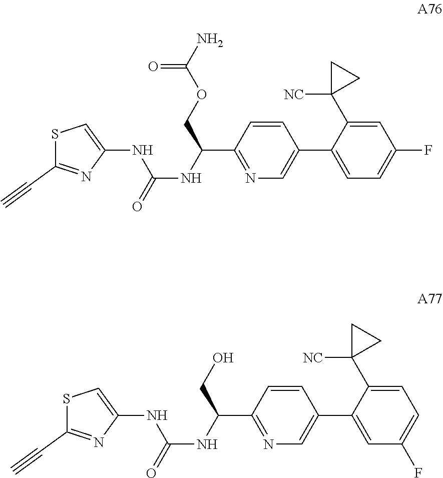 [SMILES: C#Cc1nc(NC(=O)N[C@@H](COC(N)=O)c2ccc(-c3ccc(F)cc3C3(C#N)CC3)cn2)cs1 ; C#Cc1nc(NC(=O)N[C@@H](CO)c2ccc(-c3cc(F)ccc3C3(C#N)CC3)cn2)cs1]

(R)-1-(1-(5-(2-(1-Cyanocyclopropyl)-6-fluorophenyl)pyridin-2-yl)-2-hydroxy-ethyl)-3-(2-ethynylthiazol-4-yl)urea A78. ¹H NMR (400 MHz, DMSO-d₆) δ 9.67 (s, 1H), 8.59 (d, J=2.2 Hz, 1H), 7.84 (dd, J=8.0, 2.3 Hz, 1H), 7.58-7.48 (m, 2H), 7.41 (ddd, J=17.5, 8.0, 1.3 Hz, 2H), 7.31 (s, 1H), 7.24-7.17 (m, 1H), 5.04 (t, J=5.2 Hz, 1H), 4.97 (dd, J=8.3, 4.9 Hz, 1H), 4.90 (s, 1H), 3.82 (dt, J=10.1, 5.0 Hz, 1H), 3.73 (dt, J=10.5, 5.3 Hz, 1H), 1.44-1.34 (m, 2H), 1.31 (t, J=3.7 Hz, 2H); MS (ESI) m/z: 448.2 [M+H⁺].

(R)-1-(1-(5-(2-(1-Cyanocyclopropyl)-4,6-difluorophenyl)pyridin-2-yl)-2-hydroxyethyl)-3-(2-ethynylthiazol-4-yl)urea A79. ¹H NMR (400 MHz, DMSO-d₆) δ 9.66 (s, 1H), 8.58 (d, J=2.0 Hz, 1H), 7.84 (dd, J=8.1, 2.2 Hz, 1H), 7.55-7.45 (m, 2H), 7.40 (dd, J=9.0, 2.1 Hz, 1H), 7.31 (s, 1H), 7.20 (d, J=8.1 Hz, 1H), 5.03 (t, J=5.2 Hz, 1H), 4.99-4.92 (m, 1H), 4.89 (s, 1H), 3.82 (dt, J=10.1, 5.1 Hz, 1H), 3.73 (dt, J=10.6, 5.4 Hz, 1H), 1.41 (s, 4H); MS (ESI) m/z: 466.1 [M+H⁺].

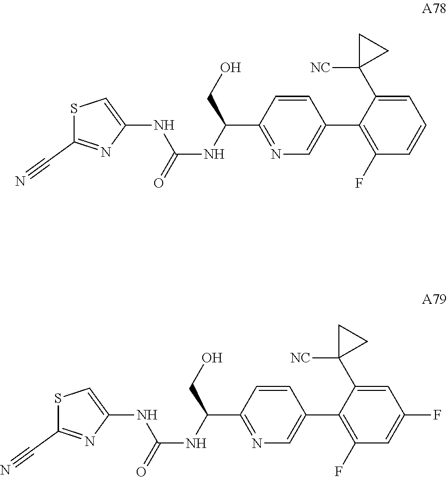 [SMILES: N#Cc1nc(NC(=O)N[C@@H](CO)c2ccc(-c3c(F)cccc3C3(C#N)CC3)cn2)cs1 ; N#Cc1nc(NC(=O)N[C@@H](CO)c2ccc(-c3c(F)cc(F)cc3C3(C#N)CC3)cn2)cs1]

1-((1R)-1-(5-(2-(2,2-Difluorocyclopropyl)phenyl)pyridin-2-yl)-2-hydroxyethyl)-3-(2-ethynylthiazol-4-yl)urea A80. ¹H NMR (400 MHz, DMSO-d₆) δ 9.65 (s, 1H), 8.57 (d, J=1.9 Hz, 1H), 7.88-7.71 (m, 1H), 7.45 (d, J=8.7 Hz, 1H), 7.44-7.34 (m, 4H), 7.30 (d, J=2.0 Hz, 1H), 7.18 (d, J=8.2 Hz, 1H), 5.04-4.83 (m, 3H), 3.82-3.63 (m, 2H), 2.90 (dd, J=23.1, 10.2 Hz, 1H), 1.83 (dd, J=17.9, 10.1 Hz, 2H); MS (ESI) m/z: 441.1 [M+H⁺].

1-((1R)-1-(5-(2-(2,2-Difluorocyclopropyl)-4-fluorophenyl)pyridin-2-yl)-2-hydroxyethyl)-3-(2-ethynylthiazol-4-yl)urea A81. ¹H NMR (400 MHz, DMSO-d₆) δ 9.65 (d, J=2.3 Hz, 1H), 8.55 (d, J=2.0 Hz, 1H), 7.78 (ddd, J=8.0, 4.4, 2.3 Hz, 1H), 7.45 (d, J=8.1 Hz, 1H), 7.41 (dd, J=8.6, 6.2 Hz, 1H), 7.29 (d, J=2.1 Hz, 1H), 7.26 (d, J=9.2 Hz, 2H), 7.17 (dd, J=8.1, 2.2 Hz, 1H), 4.99 (t, J=4.6 Hz, 1H), 4.92 (dd, J=5.3, 2.8 Hz, 1H), 4.89 (s, 1H), 3.78 (dd, J=9.8, 5.0 Hz, 1H), 3.75-3.65 (m, 1H), 3.01-2.73 (m, 1H), 2.10-1.94 (m, 1H), 1.85 (dd, J=12.4, 5.8 Hz, 1H); MS (ESI) m/z: 459.2 [M+H⁺].

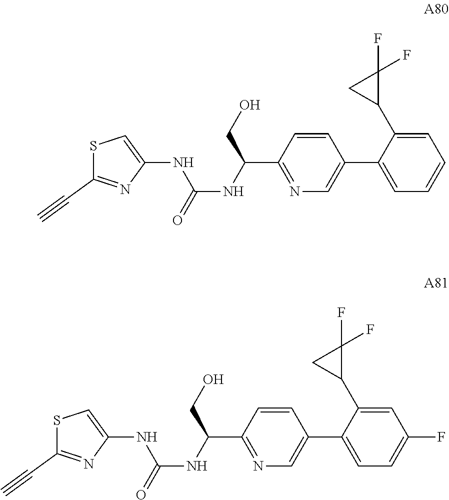 [SMILES: C#Cc1nc(NC(=O)N[C@@H](CO)c2ccc(-c3ccccc3C3CC3(F)F)cn2)cs1 ; C#Cc1nc(NC(=O)N[C@@H](CO)c2ccc(-c3ccc(F)cc3C3CC3(F)F)cn2)cs1]

1-((1R)-1-(5-(2-(2,2-Difluorocyclopropyl)-4-fluorophenyl)pyridin-2-yl)-2-hydroxyethyl)-3-(2-ethynylthiazol-4-yl)-1-methylurea A82. ¹H NMR (400 MHz, DMSO-d₆) δ 9.66 (s, 1H), 8.54 (d, J=1.0 Hz, 1H), 7.79 (dd, J=8.1, 1.9 Hz, 1H), 7.47 (s, 1H), 7.44-7.37 (m, 2H), 7.31-7.22 (m, 2H), 5.55 (dd, J=8.6, 5.4 Hz, 1H), 5.01-4.83 (m, 2H), 4.10 (dt, J=11.0, 5.4 Hz, 1H), 4.02-3.86 (m, 1H), 2.98-2.86 (m, 4H), 2.04-1.78 (m, 2H); MS (ESI) m/z: 473.1 [M+H⁺].

(2R)-2-(5-(2-(2,2-Difluorocyclopropyl)-4-fluorophenyl)pyridin-2-yl)-2-(3-(2-ethynylthiazol-4-yl)ureido)ethyl carbamate A83. ¹H NMR (400 MHz, DMSO-d₆) δ 9.64 (s, 1H), 8.58 (s, 1H), 7.82 (ddd, J=7.8, 5.3, 2.3 Hz, 1H), 7.51 (d, J=8.1 Hz, 1H), 7.42 (dd, J=9.0, 6.0 Hz, 1H), 7.31 (d, J=1.5 Hz, 1H), 7.30-7.23 (m, 2H), 7.17 (d, J=8.3 Hz, 1H), 6.53 (s, 2H), 5.17 (dd, J=13.4, 7.1 Hz, 1H), 4.25 (qd, J=11.1, 6.4 Hz, 2H), 2.91 (ddd, J=20.3, 12.2, 8.1 Hz, 1H), 1.87 (ddd, J=22.7, 19.9, 10.5 Hz, 2H); MS (ESI) m/z: 502.1 [M+H⁺].

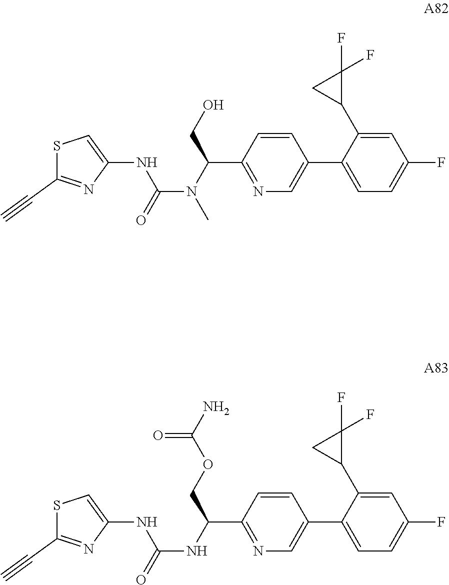 [SMILES: C#Cc1nc(NC(=O)N(C)[C@@H](CO)c2ccc(-c3ccc(F)cc3C3CC3(F)F)cn2)cs1 ; C#Cc1nc(NC(=O)N[C@@H](COC(N)=O)c2ccc(-c3ccc(F)cc3C3CC3(F)F)cn2)cs1]

(2R)-2-(5-(2-(2,2-Difluorocyclopropyl)-4-fluorophenyl)pyridin-2-yl)-2-(3-(2-ethynylthiazol-4-yl)-1-methylureido)ethyl carbamate A84. ¹H NMR (400 MHz, DMSO-d₆) δ 9.74 (s, 1H), 8.57 (t, J=2.3 Hz, 1H), 7.81 (d, J=1.8 Hz, 1H), 7.50 (s, 1H), 7.43 (d, J=9.3 Hz, 2H), 7.26 (d, J=9.4 Hz, 2H), 6.60 (s, 2H), 5.83 (dd, J=9.4, 4.9 Hz, 1H), 4.90 (s, 1H), 4.70 (dd, J=11.8, 5.0 Hz, 1H), 4.51-4.41 (m, 1H), 3.02-2.80 (m, 4H), 2.03-1.77 (m, 2H); MS (ESI) m/z: 516.1 [M+H⁺].

1-((1R)-1-(5-(2-(2,2-Difluorocyclopropyl)-4-fluorophenyl)pyridin-2-yl)-2-hydroxyethyl)-3-(5-ethynyl-1,3,4-thiadiazol-2-yl)urea A85. ¹H NMR (400 MHz, DMSO-d₆) δ 11.35 (s, 1H), 8.56 (s, 1H), 7.80 (ddd, J=8.1, 5.7, 2.3 Hz, 1H), 7.49 (d, J=8.0 Hz, 2H), 7.41 (dd, J=8.9, 6.0 Hz, 1H), 7.28 (dd, J=12.7, 6.0 Hz, 2H), 5.05 (d, J=13.6 Hz, 2H), 4.96 (d, J=8.0 Hz, 1H), 3.85-3.66 (m, 2H), 2.99-2.81 (m, 1H), 2.06-1.78 (m, 2H); MS (ESI) m/z: 460.1 [M+H⁺].

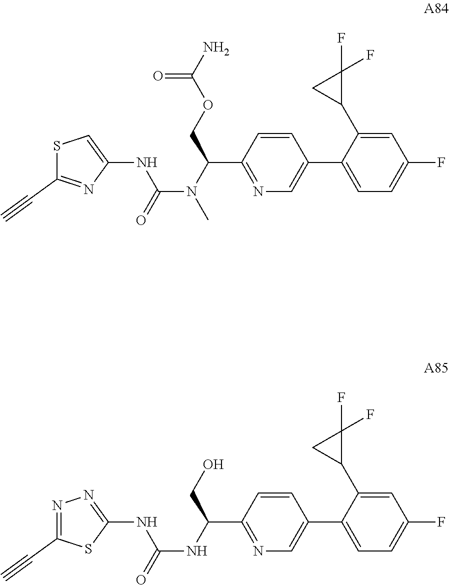 [SMILES: C#Cc1nc(NC(=O)N(C)[C@@H](COC(N)=O)c2ccc(-c3ccc(F)cc3C3CC3(F)F)cn2)cs1 ; C#Cc1nnc(NC(=O)N[C@@H](CO)c2ccc(-c3ccc(F)cc3C3CC3(F)F)cn2)s1]

1-((1R)-1-(5-(2-(2,2-Difluorocyclopropyl)-4-fluorophenyl)pyridin-2-yl)-2-hydroxyethyl)-3-(5-ethynyl-1,3,4-thiadiazol-2-yl)-1-methylurea A86. ¹H NMR (400 MHz, DMSO-d₆) δ 11.78 (s, 1H), 8.55 (s, 2H), 7.80 (d, J=7.7 Hz, 3H), 7.42 (dd, J=19.5, 6.6 Hz, 5H), 7.25 (s, 6H), 5.62 (s, 2H), 5.02 (s, 4H), 4.11 (d, J=6.5 Hz, 3H), 3.97 (d, J=10.2 Hz, 2H), 2.96 (s, 7H), 1.96 (s, 3H), 1.84 (s, 2H); MS (ESI) m/z: 474.3 [M+H⁺].

(2R)-2-(5-(2-(2,2-Difluorocyclopropyl)-4-fluorophenyl)pyridin-2-yl)-2-(3-(5-ethynyl-1,3,4-thiadiazol-2-yl)ureido)ethyl carbamate A87. ¹H NMR (400 MHz, DMSO-d₆) δ 11.41 (s, 1H), 8.59 (t, J=2.5 Hz, 1H), 7.84 (ddd, J=8.0, 7.0, 2.3 Hz, 1H), 7.56 (d, J=8.1 Hz, 2H), 7.48-7.35 (m, 1H), 7.29 (dd, J=12.5, 6.0 Hz, 2H), 6.56 (s, 2H), 5.22 (dd, J=13.1, 7.1 Hz, 1H), 5.05 (s, 1H), 4.43-4.14 (m, 2H), 3.05-2.80 (m, 1H), 2.10-1.67 (m, 2H); MS (ESI) m/z: 503.1 [M+H⁺].

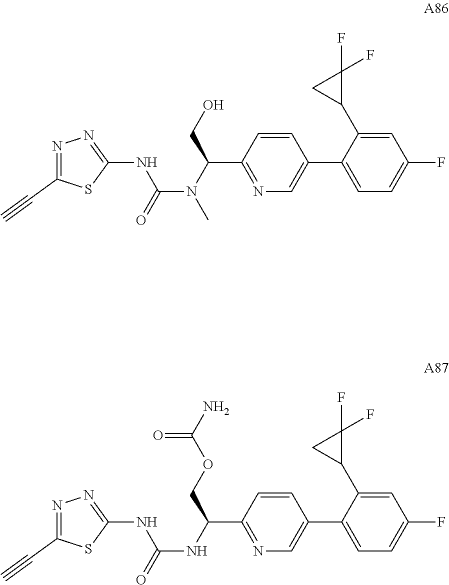 [SMILES: C#Cc1nnc(NC(=O)N(C)[C@@H](CO)c2ccc(-c3ccc(F)cc3C3CC3(F)F)cn2)s1 ; C#Cc1nnc(NC(=O)N[C@@H](COC(N)=O)c2ccc(-c3ccc(F)cc3C3CC3(F)F)cn2)s1]

(R)-1-(2-Ethynylthiazol-4-yl)-3-(2-hydroxy-1-(4-(6-(pyrrolidin-1-yl)pyridin-2-yl)phenyl)ethyl)urea A88. ¹H NMR (400 MHz, DMSO-d₆) δ 9.49 (s, 1H), 7.98 (d, J=8.4 Hz, 2H), 7.54 (dd, J=8.2, 7.5 Hz, 1H), 7.36 (d, 1=8.3 Hz, 2H), 7.26 (s, 1H), 7.06 (dd, J=11.9, 7.6 Hz, 2H), 6.39 (d, J=8.3 Hz, 1H), 5.03 (s, 1H), 4.89 (s, 1H), 4.79 (dd, J=12.7, 5.3 Hz, 1H), 3.65 (ddd, J=33.2, 10.8, 5.3 Hz, 2H), 3.33 (s, 4H), 2.04-1.88 (m, 4H); MS (ESI) m/z: 434.2 [M+H⁺].

(R)-1-(2-Ethynylthiazol-4-yl)-3-(2-hydroxy-1-(6-(pyrrolidin-1-yl)-[2,3′-bipyridin]-6′-yl)ethyl)urea A89. ¹H NMR (400 MHz, DMSO-d₆) δ 9.65 (s, 1H), 9.17 (d, J=1.8 Hz, 1H), 8.33 (dd, J=8.2, 2.3 Hz, 1H), 7.59 (dd, J=8.2, 7.5 Hz, 1H), 7.41 (d, J=8.1 Hz, 1H), 7.28 (s, 1H), 7.15 (dd, J=7.6, 4.3 Hz, 2H), 6.46 (d, J=8.4 Hz, 1H), 4.98 (s, 1H), 4.92-4.80 (m, 2H), 3.74 (d, J=22.3 Hz, 2H), 3.47 (t, J=6.4 Hz, 4H), 1.97 (dd, J=8.0, 5.2 Hz, 4H); MS (ESI) m/z: 435.2 [M+H⁺].

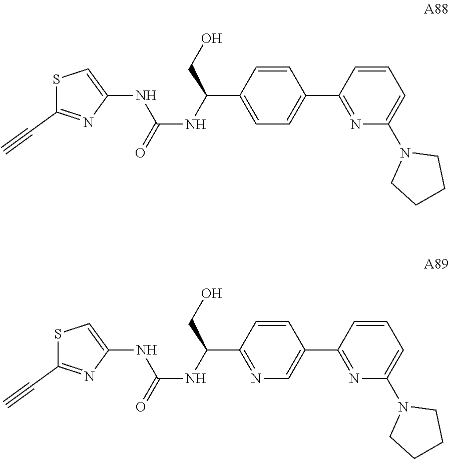 [SMILES: C#Cc1nc(NC(=O)N[C@@H](CO)c2ccc(-c3cccc(N4CCCC4)n3)cc2)cs1 ; C#Cc1nc(NC(=O)N[C@@H](CO)c2ccc(-c3cccc(N4CCCC4)n3)cn2)cs1]

(R)-2-(3-(2-Ethynylthiazol-5-yl)ureido)-2-(6-(pyrrolidin-1-yl)-[2,3′-bipyridin]-6′-yl)ethyl carbamate A90. ¹H NMR (400 MHz, DMSO-d₆) δ 9.43 (s, 1H), 9.16 (s, 1H), 8.33 (d, J=8.1 Hz, 1H), 7.56 (t, J=7.8 Hz, 1H), 7.45 (d, J=8.2 Hz, 1H), 7.27 (s, 1H), 7.12 (d, J=7.3 Hz, 1H), 7.04 (d, J=8.3 Hz, 1H), 6.45 (d, J=8.3 Hz, 1H), 6.17 (s, 2H), 5.14 (d, J=7.3 Hz, 1H), 4.64 (s, 1H), 4.29 (d, J=5.8 Hz, 2H), 3.49 (d, J=6.4 Hz, 4H), 1.98 (t, J=6.5 Hz, 4H); MS (ESI) m/z: 478.1 [M+H⁺].

(R)-3-(2-Ethynylthiazol-4-yl)-1-(2-hydroxy-1-(6-(pyrrolidin-1-yl)-[2,3′-bipyridin]-6′-yl)ethyl)-1-methylurea A91. ¹H NMR (400 MHz, DMSO-d₆) δ 9.66 (s, 1H), 9.17 (dd, J=2.3, 0.7 Hz, 1H), 8.35 (dd, J=8.2, 2.3 Hz, 1H), 7.58 (dd, J=8.4, 7.4 Hz, 1H), 7.47 (s, 1H), 7.39 (d, J=8.2 Hz, 1H), 7.16 (d, J=7.4 Hz, 1H), 6.45 (d, J=8.4 Hz, 1H), 5.50 (dd, J=8.9, 5.4 Hz, 1H), 4.93 (s, 1H), 4.90 (s, 1H), 4.09 (dd, J=11.3, 5.5 Hz, 1H), 3.93 (t, J=10.4 Hz, 1H), 3.50-3.42 (m, 4H), 2.92 (s, 3H), 2.02-1.91 (m, 4H); MS (ESI) m/z: 449 [M+H⁺].

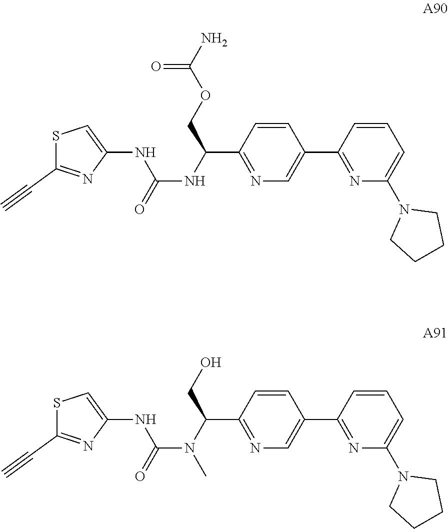 [SMILES: C#Cc1nc(NC(=O)N[C@@H](COC(N)=O)c2ccc(-c3cccc(N4CCCC4)n3)cn2)cs1 ; C#Cc1nc(NC(=O)N(C)[C@@H](CO)c2ccc(-c3cccc(N4CCCC4)n3)cn2)cs1]

(R)-2-(3-(2-Ethynylthiazol-4-yl)-1-methylureido)-2-(6-(pyrrolidin-1-yl)-[2,3′-bipyridin]-6′-yl)ethyl carbamate A92. ¹H NMR (400 MHz, DMSO-d₆) δ 9.73 (s, 1H), 9.19 (d, J=1.8 Hz, 1H), 8.38 (dd, J=8.2, 2.3 Hz, 1H), 7.59 (dd, J=8.3, 7.5 Hz, 1H), 7.41 (d, J=8.3 Hz, 1H), 7.17 (d, J=7.3 Hz, 1H), 6.58 (s, 1H), 6.47 (d, J=8.4 Hz, 2H), 4.90 (s, 1H), 4.68 (dd, J=11.7, 5.2 Hz, 1H), 4.48 (dd, J=11.6, 9.3 Hz, 1H), 3.46 (t, J=6.4 Hz, 5H), 2.89 (s, 3H), 1.97 (dd, J=7.9, 5.3 Hz, 5H); MS (ESI) m/z: 492.2 [M+H⁺].

(R)-1-(5-Ethynyl-1,3,4-thiadiazol-2-yl)-3-(2-hydroxy-1-(4-(6-(pyrrolidin-1-yl)-pyridin-2-yl)phenyl)ethyl)urea A93. ¹H NMR (400 MHz, DMSO-d₆) δ 11.20 (s, 1H), 7.99 (d, J=8.3 Hz, 2H), 7.55 (t, J=7.9 Hz, 1H), 7.37 (d, J=8.3 Hz, 3H), 7.07 (d, J=7.4 Hz, 1H), 6.39 (d, J=8.3 Hz, 1H), 5.13 (s, 1H), 5.03 (s, 1H), 4.90-4.71 (m, 1H), 3.78-3.57 (m, 2H), 3.46 (t, J=6.3 Hz, 4H), 1.96 (t, J=6.6 Hz, 4H); MS (ESI) m/z: 435.1 [M+H⁺].

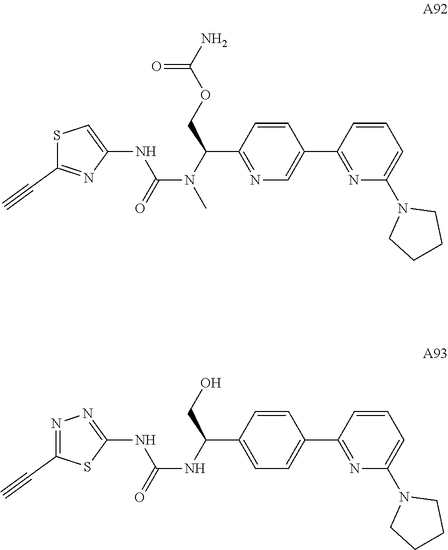 [SMILES: C#Cc1nc(NC(=O)N(C)[C@@H](COC(N)=O)c2ccc(-c3cccc(N4CCCC4)n3)cn2)cs1 ; C#Cc1nnc(NC(=O)N[C@@H](CO)c2ccc(-c3cccc(N4CCCC4)n3)cc2)s1]

(R)-2-(3-(5-Ethynyl-1,3,4-thiadiazol-2-yl)ureido)-2-(4-(6-(pyrrolidin-1-yl)-pyridin-2-yl)phenyl)ethyl carbamate A94. ¹H NMR (400 MHz, DMSO-d₆) δ 11.32 (s, 1H), 8.02 (d, J=8.3 Hz, 2H), 7.60-7.37 (m, 4H), 7.09 (d, J=7.4 Hz, 1H), 6.60 (s, 2H), 6.40 (d, J=8.3 Hz, 1H), 5.19-4.87 (m, 2H), 4.21 (d, J=6.0 Hz, 2H), 3.46 (t, J=6.4 Hz, 4H), 1.96 (t, J=6.6 Hz, 4H); MS (ESI) m/z: 478.2 [M+H⁺].

(R)-2-(3-(5-Ethynyl-1,3,4-thiadiazol-2-yl)-1-methylureido)-2-(4-(6-(pyrrolidin-1-yl)pyridin-2-yl)phenyl)ethyl carbamate A95. ¹H NMR (400 MHz, DMSO-d₆) δ 11.87 (s, 1H), 8.08-8.00 (m, 2H), 7.55 (dd, J=8.3, 7.4 Hz, 1H), 7.42-7.36 (m, 2H), 7.10 (d, J=7.4 Hz, 1H), 6.58 (d, J=48.1 Hz, 2H), 6.41 (d, J=8.4 Hz, 1H), 5.76 (s, 1H), 5.03 (s, 1H), 4.60-4.40 (m, 2H), 3.45 (q, J=5.0, 3.7 Hz, 4H), 2.88 (s, 3H), 2.02-1.91 (m, 4H); MS (ESI) m/z: 492 [M+H⁺].

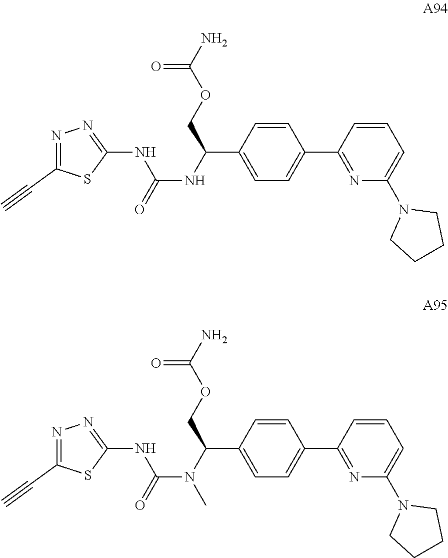 [SMILES: C#Cc1nnc(NC(=O)N[C@@H](COC(N)=O)c2ccc(-c3cccc(N4CCCC4)n3)cc2)s1 ; C#Cc1nnc(NC(=O)N(C)[C@@H](COC(N)=O)c2ccc(-c3cccc(N4CCCC4)n3)cc2)s1]

(R)-1-(5-Ethynyl-1,3,4-thiadiazol-2-yl)-3-(2-hydroxy-1-(6-(pyrrolidin-1-yl)-[2,3′-bipyridin]-6′-yl)ethyl)urea A96. ¹H NMR (400 MHz, DMSO-d₆) δ 11.35 (s, 1H), 9.18 (d, J=2.2 Hz, 1H), 8.35 (dd, J=8.2, 2.3 Hz, 1H), 7.59 (dd, J=8.4, 7.4 Hz, 1H), 7.46 (dd, J=13.5, 8.0 Hz, 2H), 7.17 (d, J=7.3 Hz, 1H), 6.46 (d, J=8.4 Hz, 1H), 5.05 (d, J=8.8 Hz, 2H), 4.91 (dt, J=7.9, 5.2 Hz, 1H), 3.76 (dh, J=16.1, 5.3 Hz, 2H), 3.46 (d, J=6.6 Hz, 4H), 2.02-1.93 (m, 4H); MS (ESI) m/z: 436 [M+H⁺].

(R)-2-(3-(5-Ethynyl-1,3,4-thiadiazol-2-yl)ureido)-2-(6-(pyrrolidin-1-yl)-[2,3′-bipyridin]-6′-yl)ethyl carbamate A97. ¹H NMR (400 MHz, DMSO-d₆) δ 11.41 (s, 1H), 9.21 (d, J=1.8 Hz, 1H), 8.39 (dd, J=8.2, 2.2 Hz, 1H), 7.65-7.56 (m, 1H), 7.55-7.47 (m, 2H), 7.19 (d, J=7.3 Hz, 1H), 6.54 (s, 2H), 6.47 (d, J=8.4 Hz, 1H), 5.17 (dd, J=13.8, 6.0 Hz, 1H), 5.05 (s, 1H), 4.29 (d, J=5.9 Hz, 2H), 3.47 (t, J=6.3 Hz, 4H), 1.97 (t, J=6.6 Hz, 4H); MS (ESI) m/z: 479.2 [M+H⁺].

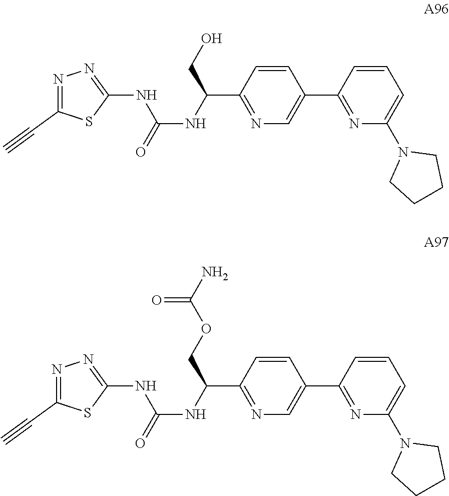 [SMILES: C#Cc1nnc(NC(=O)N[C@@H](CO)c2ccc(-c3cccc(N4CCCC4)n3)cn2)s1 ; C#Cc1nnc(NC(=O)N[C@@H](COC(N)=O)c2ccc(-c3cccc(N4CCCC4)n3)cn2)s1]

(R)-1-(1-(4-(2-(Dimethylamino)pyridin-3-yl)phenyl)-2-hydroxyethyl)-3-(2-ethynylthiazol-4-yl)urea A98. ¹H NMR (400 MHz, DMSO-d₆) δ 9.48 (s, 1H), 8.17 (s, 1H), 8.13 (dd, J=4.8, 1.9 Hz, 1H), 7.50-7.41 (m, 3H), 7.37 (d, J=8.2 Hz, 2H), 7.27 (s, 1H), 7.03 (d, J=8.0 Hz, 1H), 6.88 (dd, J=7.4, 4.8 Hz, 1H), 5.04 (s, 1H), 4.81 (dd, J=13.0, 5.3 Hz, 1H), 3.66 (dd, J=27.6, 7.8 Hz, 2H), 2.62 (s, 6H); MS (ESI) m/z: 408.2 [M+H⁺].

(R)-2-(4-(2-(Dimethylamino)pyridin-3-yl)phenyl)-2-(3-(2-ethynylthiazol-4-yl)-ureido)ethyl carbamate A9. ¹H NMR (400 MHz, DMSO-d₆) δ 9.48 (s, 1H), 8.13 (dd, J=4.8, 1.9 Hz, 1H), 7.50-7.45 (m, 3H), 7.41 (d, J=8.3 Hz, 2H), 7.29 (s, 1H), 7.04 (d, J=8.3 Hz, 1H), 6.89 (dd, J=7.4, 4.8 Hz, 1H), 6.57 (s, 1H), 5.04 (d, J=5.0 Hz, 1H), 4.90 (s, 1H), 4.16 (ddd, J=18.8, 11.2, 6.3 Hz, 2H), 2.62 (s, 6H); MS (ESI) m/z: 451.1 [M+H⁺].

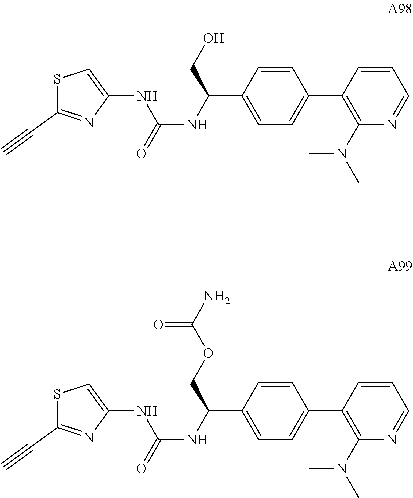 [SMILES: C#Cc1nc(NC(=O)N[C@@H](CO)c2ccc(-c3cccnc3N(C)C)cc2)cs1 ; C#Cc1nc(NC(=O)N[C@@H](COC(N)=O)c2ccc(-c3cccnc3N(C)C)cc2)cs1]

(R)-1-(1-(3-Chloro-4-(2-(dimethylamino)pyridin-3-yl)phenyl)-2-hydroxyethyl)-3-(2-ethynylthiazol-4-yl)urea A100. ¹H NMR (400 MHz, DMSO-d₆) δ 9.51 (s, 1H), 8.21 (s, 1H), 8.16 (dd, J=4.8, 1.9 Hz, 1H), 7.48 (s, 1H), 7.37-7.32 (m, 3H), 7.28 (s, 1H), 7.10 (d, J=8.0 Hz, 1H), 6.83 (dd, J=7.4, 4.8 Hz, 1H), 5.10 (s, 1H), 4.90 (s, 1H), 4.85-4.78 (m, 1H), 3.76-3.60 (m, 2H), 2.62 (s, 6H); MS (ESI) m/z: 442.2 [M+H⁺].

(R)-2-(3-Chloro-4-(2-(dimethylamino)pyridin-3-yl)phenyl)-2-(3-(2-ethynyl-thiazol-4-yl)ureido)ethyl carbamate A101. ¹H NMR (400 MHz, DMSO-d₆) δ 9.56 (s, 1H), 8.17 (dd, J=4.8, 1.9 Hz, 1H), 7.54 (d, J=1.5 Hz, 1H), 7.43-7.34 (m, 3H), 7.30 (s, 1H), 7.12 (d, J=8.2 Hz, 1H), 6.83 (dd, J=7.4, 4.8 Hz, 1H), 6.61 (s, 2H), 5.06 (td, J=7.4, 4.9 Hz, 1H), 4.90 (s, 1H), 4.18 (qd, J=11.3, 6.0 Hz, 2H), 2.62 (s, 6H); MS (ESI) m/z: 485 [M+H⁺].

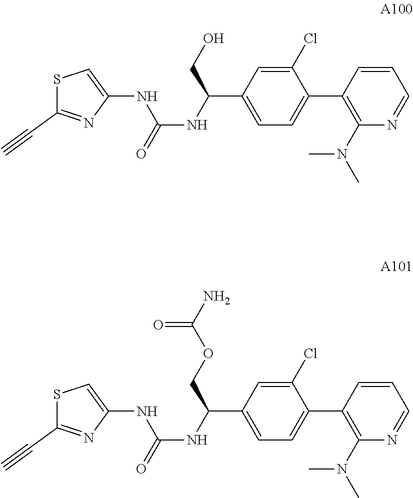 [SMILES: C#Cc1nc(NC(=O)N[C@@H](CO)c2ccc(-c3cccnc3N(C)C)c(Cl)c2)cs1 ; C#Cc1nc(NC(=O)N[C@@H](COC(N)=O)c2ccc(-c3cccnc3N(C)C)c(Cl)c2)cs1]

(R)-1-(1-(4-(2-(3,3-Difluoroazetidin-1-yl)pyridin-3-yl)phenyl)-2-hydroxyethyl)-3-(2-ethynylthiazol-4-yl)urea A102. ¹H NMR (400 MHz, DMSO-d₆) δ 9.50 (s, 1H), 8.20 (dd, J=4.9, 1.7 Hz, 1H), 7.53 (dd, J=7.4, 1.8 Hz, 1H), 7.39 (s, 4H), 7.27 (s, 1H), 7.05 (d, J=7.9 Hz, 1H), 6.98 (dd, J=7.4, 4.9 Hz, 1H), 5.03 (t, J=5.3 Hz, 1H), 4.90 (s, 1H), 4.82 (dd, J=5.1, 2.7 Hz, 1H), 3.94 (t, J=12.7 Hz, 4H), 3.67 (ddd, J=20.8, 10.9, 5.6 Hz, 2H); MS (ESI) m/z: 456.1 [M+H⁺].

(R)-2-(4-(2-(3,3-Difluoroazetidin-1-yl)pyridin-3-yl)phenyl)-2-(3-(2-ethynyl-thiazol-4-yl)ureido)ethyl carbamate A103. ¹H NMR (400 MHz, DMSO-d₆) δ 9.50 (s, 1H), 8.20 (dd, J=4.9, 1.7 Hz, 1H), 7.54 (dd, J=7.4, 1.8 Hz, 1H), 7.48-7.37 (m, 4H), 7.29 (s, 1H), 7.06 (d, J=8.2 Hz, 1H), 6.98 (dd, J=7.4, 4.9 Hz, 1H), 6.58 (s, 2H), 5.06 (td, J=7.7, 4.8 Hz, 1H), 4.89 (s, 1H), 4.21 (dd, J=11.3, 4.9 Hz, 1H), 4.14 (dd, J=11.3, 7.5 Hz, 1H), 3.95 (d, J=25.3 Hz, 4H); MS (ESI) m/z: 499.1 [M+H⁺].

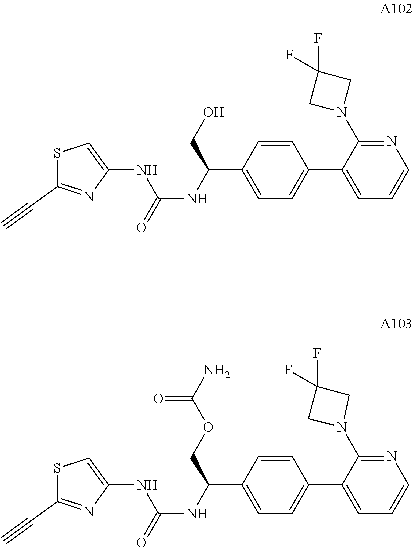 [SMILES: C#Cc1nc(NC(=O)N[C@@H](CO)c2ccc(-c3cccnc3N3CC(F)(F)C3)cc2)cs1 ; C#Cc1nc(NC(=O)N[C@@H](COC(N)=O)c2ccc(-c3cccnc3N3CC(F)(F)C3)cc2)cs1]

(R)-1-(2-Ethynylthiazol-4-yl)-3-(2-hydroxy-1-(4-(1-hydroxyisoquinolin-8-yl)-phenyl)ethyl)urea A104. ¹H NMR (400 MHz, DMSO-d₆) δ 10.90 (d, J=5.5 Hz, 1H), 9.47 (s, 1H), 7.76-7.54 (m, 2H), 7.30 (s, 1H), 7.26 (d, J=8.2 Hz, 2H), 7.21 (d, J=8.3 Hz, 2H), 7.18-7.12 (m, 2H), 7.01 (d, J=8.1 Hz, 1H), 6.56 (dd, J=7.0, 1.3 Hz, 1H), 5.06 (t, J=5.1 Hz, 1H), 4.89 (s, 1H), 4.83 (dd, J=13.2, 5.5 Hz, 1H), 3.68 (ddd, J=29.7, 10.8, 5.5 Hz, 2H); MS (ESI) m/z: 431.1 [M+H⁺].

(R)-2-(3-(2-Ethynylthiazol-4-yl)ureido)-2-(4-(1-hydroxyisoquinolin-8-yl)phenyl)-ethyl carbamate A105. ¹H NMR (400 MHz, DMSO-d₆) δ 10.91 (d, J=5.8 Hz, 1H), 9.50 (s, 1H), 7.70-7.61 (m, 2H), 7.30 (d, J=7.5 Hz, 3H), 7.24 (d, J=8.3 Hz, 2H), 7.16 (td, J=5.2, 2.8 Hz, 2H), 7.04 (dd, J=8.5, 3.4 Hz, 1H), 6.57 (d, J=7.0 Hz, 3H), 5.07 (td, J=7.8, 4.8 Hz, 1H), 4.90 (s, 1H), 4.24 (dd, J=11.2, 4.9 Hz, 1H), 4.15 (dd, J=11.2, 7.6 Hz, 1H); MS (ESI) m/z: 474 [M+H⁺].

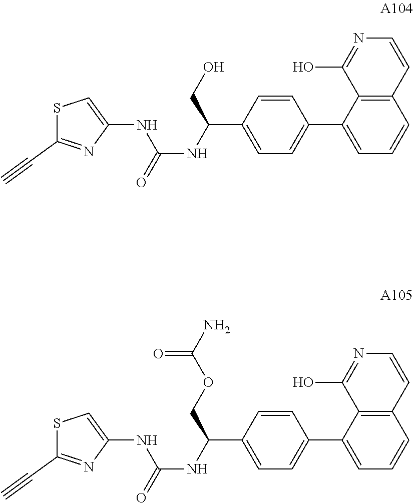 [SMILES: C#Cc1nc(NC(=O)N[C@@H](CO)c2ccc(-c3cccc4ccnc(O)c34)cc2)cs1 ; C#Cc1nc(NC(=O)N[C@@H](COC(N)=O)c2ccc(-c3cccc4ccnc(O)c34)cc2)cs1]

(R)-1-(2-Ethynylthiazol-4-yl)-3-(2-hydroxy-1-(5-(1-hydroxyisoquinolin-8-yl)-pyridin-2-yl)ethyl)urea A106. ¹H NMR (400 MHz, DMSO-d₆) δ 11.02 (d, J=5.5 Hz, 1H), 9.64 (s, 1H), 8.43 (d, J=1.9 Hz, 1H), 7.71 (d, J=4.1 Hz, 2H), 7.66 (dd, J=8.0, 2.2 Hz, 1H), 7.37-7.29 (m, 2H), 7.26-7.19 (m, 2H), 7.20-7.08 (m, 2H), 6.61 (dd, J=7.1, 1.2 Hz, 1H), 5.02 (s, 1H), 4.96-4.85 (m, 2H), 3.76 (dt, J=10.1, 5.4 Hz, 2H); MS (ESI) m/z: 432.1 [M+H⁺].

(R)-1-(1-(3-Chloro-4-(1-hydroxyisoquinolin-8-yl)phenyl)-2-hydroxyethyl)-3-(2-ethynylthiazol-4-yl)urea A107. ¹H NMR (400 MHz, DMSO-d₆) δ 10.88 (s, 1H), 9.51 (s, 1H), 7.68 (d, J=4.4 Hz, 2H), 7.34 (t, J=1.8 Hz, 1H), 7.31 (d, J=1.9 Hz, 1H), 7.25 (dt, J=7.9, 1.8 Hz, 1H), 7.17 (d, J=8.2 Hz, 1H), 7.14 (dd, J=6.8, 3.9 Hz, 1H), 7.09 (dd, J=8.5, 4.2 Hz, 2H), 6.58-6.54 (m, 1H), 5.13 (s, 1H), 4.90 (d, J=0.8 Hz, 1H), 4.84 (dd, J=8.4, 4.3 Hz, 1H), 3.70 (ddd, J=13.9, 9.9, 5.4 Hz, 2H); MS (ESI) m/z: 465.1 [M+H⁺].

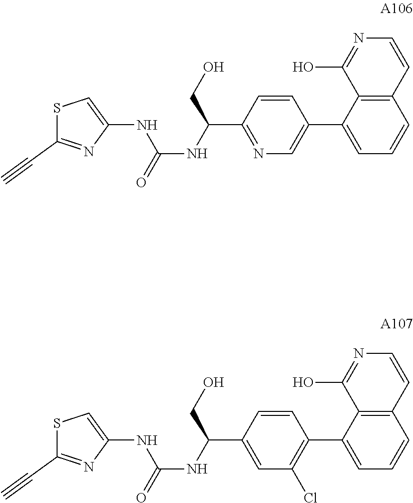 [SMILES: C#Cc1nc(NC(=O)N[C@@H](CO)c2ccc(-c3cccc4ccnc(O)c34)cn2)cs1 ; C#Cc1nc(NC(=O)N[C@@H](CO)c2ccc(-c3cccc4ccnc(O)c34)c(Cl)c2)cs1]

(R)-1-(5-Ethynyl-1,3,4-thiadiazol-2-yl)-3-(2-hydroxy-1-(4-(1-hydroxyisoquinolin-8-yl)phenyl)ethyl)urea A108. ¹H NMR (400 MHz, DMSO-d₆) δ 11.19 (s, 1H), 10.90 (d, J=5.5 Hz, 1H), 7.74-7.53 (m, 2H), 7.36 (d, J=7.8 Hz, 1H), 7.24 (dd, J=20.8, 8.3 Hz, 4H), 7.18-7.12 (m, 2H), 6.56 (dd, J=7.0, 1.3 Hz, 1H), 5.15 (t, J=4.9 Hz, 1H), 5.04 (s, 1H), 4.86 (d, J=7.3 Hz, 1H), 3.80-3.71 (m, 1H), 3.67 (dt, J=11.0, 5.5 Hz, 1H); MS (ESI) m/z: 432.1 [M+H⁺].

(R)-1-(2-Ethynylthiazol-4-yl)-3-(2-hydroxy-1-(4-(4-hydroxyquinazolin-5-yl)phenyl)ethyl)urea A109. ¹H NMR (400 MHz, DMSO-d₆) δ 11.98 (d, J=3.7 Hz, 1H), 9.48 (s, 1H), 8.06 (d, J=3.6 Hz, 1H), 7.81-7.73 (m, 1H), 7.66 (dd, J=8.2, 1.3 Hz, 1H), 7.36-7.19 (m, 6H), 7.02 (d, J=8.1 Hz, 1H), 5.07 (t, J=5.1 Hz, 1H), 4.89 (s, 1H), 4.84 (dt, J=7.9, 5.3 Hz, 1H), 3.73 (dt, J=10.0, 4.9 Hz, 1H), 3.64 (dt, J=10.8, 5.5 Hz, 1H); MS (ESI) m/z: 432.1 [M+H⁺].

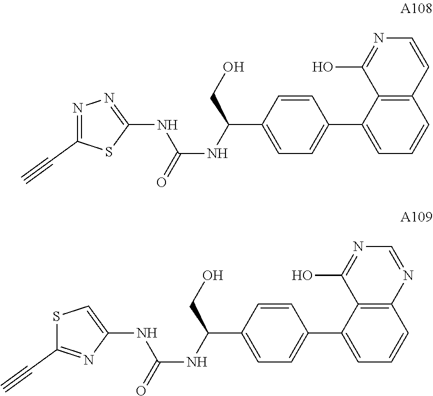 [SMILES: C#Cc1nnc(NC(=O)N[C@@H](CO)c2ccc(-c3cccc4ccnc(O)c34)cc2)s1 ; C#Cc1nc(NC(=O)N[C@@H](CO)c2ccc(-c3cccc4ncnc(O)c34)cc2)cs1]

(R)-1-(1-(3-Chloro-4-(4-hydroxyquinazolin-5-yl)phenyl)-2-hydroxyethyl)-3-(2-ethynylthiazol-4-yl)urea A110. ¹H NMR (400 MHz, DMSO-d₆) δ 12.00 (s, 1H), 9.50 (d, J=1.7 Hz, 1H), 8.05 (d, J=3.2 Hz, 1H), 7.81 (t, J=7.8 Hz, 1H), 7.71 (d, J=8.1 Hz, 1H), 7.38 (dd, J=3.5, 1.5 Hz, 1H), 7.33-7.25 (m, 2H), 7.22 (d, J=7.9 Hz, 1H), 7.17 (d, J=7.3 Hz, 1H), 7.08 (dd, J=8.1, 3.6 Hz, 1H), 5.13 (dd, J=4.9, 3.5 Hz, 1H), 4.90 (d, J=1.0 Hz, 1H), 4.87-4.81 (m, 1H), 3.70 (ddd, J=14.6, 10.3, 5.4 Hz, 2H); MS (ESI) m/z: 466.1 [M+H⁺].

(R)-1-(1-(4-(Benzo[d]thiazol-7-yl)phenyl)-2-hydroxyethyl)-3-(2-ethynylthiazol-4-yl)-1-methylurea A111. ¹H NMR (400 MHz, DMSO-d₆) δ 9.61 (s, 1H), 9.45 (s, 1H), 8.11 (dd, J=8.0, 1.1 Hz, 1H), 7.72 (d, J=8.3 Hz, 2H), 7.69-7.64 (m, 1H), 7.61-7.56 (m, 1H), 7.48 (d, J=7.2 Hz, 3H), 5.53 (dd, J=8.2, 6.0 Hz, 1H), 5.04 (t, J=5.3 Hz, 1H), 4.90 (s, 1H), 4.06-3.85 (m, 2H), 2.88 (s, 3H); MS (ESI) m/z: 435.1 [M+H⁺].

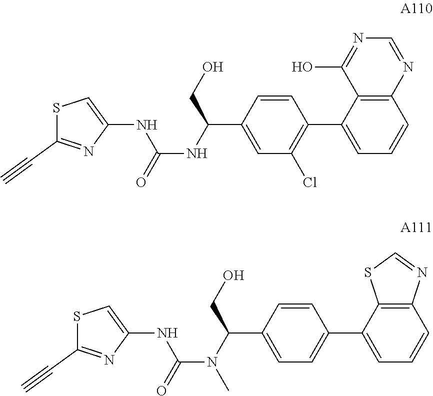 [SMILES: C#Cc1nc(NC(=O)N[C@@H](CO)c2ccc(-c3cccc4ncnc(O)c34)c(Cl)c2)cs1 ; C#Cc1nc(NC(=O)N(C)[C@@H](CO)c2ccc(-c3cccc4ncsc34)cc2)cs1]

(R)-3-(1-(4-(Benzo[d]thiazol-7-yl)phenyl)-2-hydroxyethyl)-1-(2-ethynylthiazol-4-yl)-1-methylurea A112. ¹H NMR (400 MHz, DMSO-d₆) δ 9.44 (s, 1H), 8.40 (d, J=7.4 Hz, 1H), 8.10 (d, J=7.8 Hz, 1H), 7.69 (d, J=8.2 Hz, 2H), 7.66 (d, J=8.1 Hz, 1H), 7.58 (d, J=7.3 Hz, 1H), 7.52 (d, J=8.1 Hz, 2H), 7.30 (s, 1H), 5.07 (s, 1H), 4.99 (s, 1H), 4.93 (d, J=6.7 Hz, 1H), 3.72 (s, 2H), 3.33 (s, 3H); MS (ESI) m/z: 435.1 [M+H⁺].

(R)-1-(1-(4-(Benzo[d]thiazol-7-yl)phenyl)-2-hydroxyethyl)-3-(2-ethynylthiazol-4-yl)-1,3-dimethylurea A113. ¹H NMR (400 MHz, DMSO-d₆) δ 9.46 (s, 1H), 8.12 (dd, J=8.0, 0.9 Hz, 1H), 7.73-7.66 (m, 3H), 7.60 (d, J=7.2 Hz, 1H), 7.54 (d, J=8.2 Hz, 2H), 7.26 (s, 1H), 5.31 (dd, 1=8.9, 5.8 Hz, 1H), 5.12 (t, J=5.2 Hz, 1H), 4.96 (s, 1H), 4.06-3.89 (m, 2H), 3.19 (s, 3H), 2.54 (s, 3H); MS (ESI) m/z: 449.0 [M+H⁺].

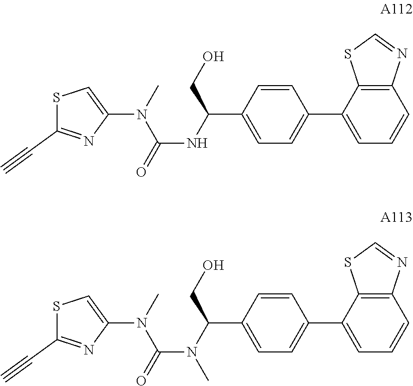 [SMILES: C#Cc1nc(N(C)C(=O)N[C@@H](CO)c2ccc(-c3cccc4ncsc34)cc2)cs1 ; C#Cc1nc(N(C)C(=O)N(C)[C@@H](CO)c2ccc(-c3cccc4ncsc34)cc2)cs1]

(R)-1-(1-(5-(Benzo[d]thiazol-7-yl)pyridin-2-yl)-2-hydroxyethyl)-3-(2-ethynyl-thiazol-4-yl)urea A114. ¹H NMR (400 MHz, DMSO-d₆) δ 9.67 (s, 1H), 9.49 (s, 1H), 8.91 (d, J=1.9 Hz, 1H), 8.19-8.11 (m, 2H), 7.74-7.64 (m, 2H), 7.54 (d, J=8.1 Hz, 1H), 7.30 (s, 1H), 7.21 (d, J=8.0 Hz, 1H), 5.05 (d, J=5.3 Hz, 1H), 4.98-4.87 (m, 2H), 3.79 (ddd, J=26.8, 10.5, 5.3 Hz, 2H); MS (ESI) m/z: 422.1 [M+H⁺].

(R)-2-(3-Chloro-4-(4-fluorobenzo[d]thiazol-7-yl)phenyl)-2-(3-(2-ethynylthiazol-4-yl)ureido)ethane-1-sulfonamide A115. ¹H NMR (400 MHz, DMSO-d₆) δ 10.55 (s, 1H), 9.46 (s, 1H), 8.59 (d, J=6.7 Hz, 1H), 8.53 (s, 3H), 7.68 (s, 1H), 7.56-7.44 (m, 4H), 7.30 (s, 1H), 7.01 (s, 2H), 5.33 (dd, J=12.8, 7.3 Hz, 1H), 4.88 (s, 1H), 3.58 (ddd, J=19.6, 14.4, 6.5 Hz, 2H); MS (ESI) m/z: 537.1 [M+H⁺].

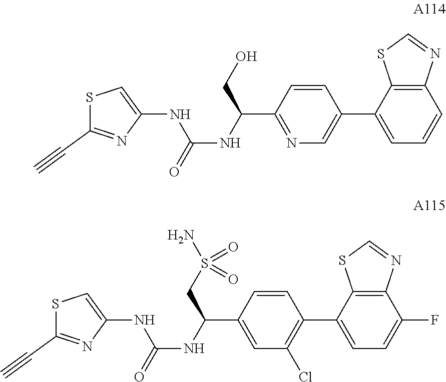 [SMILES: C#Cc1nc(NC(=O)N[C@@H](CO)c2ccc(-c3cccc4ncsc34)cn2)cs1 ; C#Cc1nc(NC(=O)N[C@@H](CS(N)(=O)=O)c2ccc(-c3ccc(F)c4ncsc34)c(Cl)c2)cs1]

(R)-2-(4-(Benzo[d]thiazol-7-yl)phenyl)-2-(3-(2-ethynylthiazol-4-yl)ureido)ethyl L-valinate A116. ¹H NMR (400 MHz, DMSO-d₆) δ 9.53 (s, 1H), 9.46 (s, 1H), 8.11 (dd, J=8.1, 0.9 Hz, 1H), 7.81-7.62 (m, 3H), 7.57 (dd, J=7.3, 5.4 Hz, 3H), 7.30 (s, 1H), 7.14 (d, J=8.3 Hz, 1H), 5.18 (d, J=8.0 Hz, 1H), 4.91 (s, 1H), 4.50-4.18 (m, 2H), 3.16 (s, 1H), 1.80 (dd, J=12.8, 6.2 Hz, 1H), 0.75 (dd, J=21.1, 6.8 Hz, 6H); MS (ESI) m/z: 520.2 [M+H⁺].

(S)-1-((R)-2-(4-(Benzo[d]thiazol-7-yl)phenyl)-2-(3-(2-ethynylthiazol-5-yl)-ureido)ethoxy)-3-methyl-1-oxobutan-2-aminium chloride A116a. ¹H NMR (400 MHz, DMSO-d₆) δ 9.68 (s, 1H), 9.46 (s, 1H), 8.49 (s, 3H), 8.12 (dd, J=8.1, 1.0 Hz, 1H), 7.74 (d, J=8.3 Hz, 3H), 7.71-7.65 (m, 1H), 7.62 (d, J=8.3 Hz, 2H), 7.58 (d, J=6.8 Hz, 1H), 7.30 (s, 1H), 5.23 (dd, J=13.8, 5.5 Hz, 1H), 4.92 (s, 1H), 4.53 (d, J=5.6 Hz, 2H), 3.92-3.79 (m, 2H), 0.86 (d, J=6.9 Hz, 3H), 0.81 (d, J=6.9 Hz, 3H); MS (ESI) m/z: 520.1 [M+H⁺].

(5Z,8Z,11Z,14Z)—(R)-2-(4-(Benzo[d]thiazol-7-yl)phenyl)-2-(3-(2-ethynylthiazol-4-yl)ureido)ethyl icosa-5,8,11,14-tetraenoate A117. ¹H NMR (400 MHz, DMSO-d₆) δ 9.49 (s, 1H), 9.45 (s, 1H), 8.11 (dd, J=8.1, 1.0 Hz, 1H), 7.73 (d, J=8.3 Hz, 2H), 7.69-7.64 (m, 1H), 7.58 (d, J=6.8 Hz, 1H), 7.54 (d, J=8.2 Hz, 2H), 7.29 (s, 1H), 7.15 (d, J=8.4 Hz, 1H), 5.39-5.22 (m, 8H), 5.17 (dd, J=13.8, 5.7 Hz, 1H), 4.90 (s, 1H), 4.42-4.27 (m, 2H), 2.73 (ddd, J=8.9, 8.3, 4.3 Hz, 6H), 2.30 (t, J=7.4 Hz, 2H), 1.99 (dt, J=13.6, 6.8 Hz, 4H), 1.55 (p, J=7.4 Hz, 2H), 1.30-1.19 (m, 6H), 0.82 (t, J=6.9 Hz, 3H); MS (ESI) m/z: 707.3 [M+H⁺].

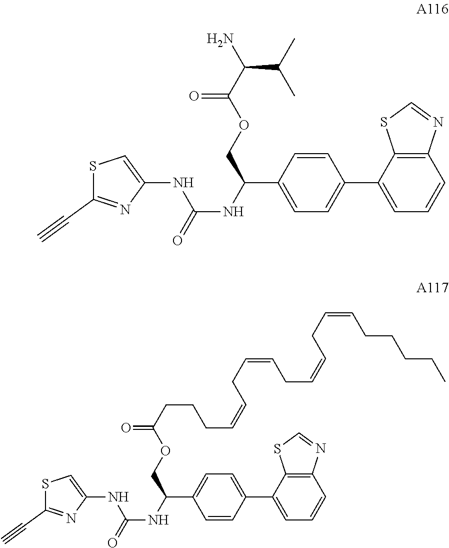 [SMILES: C#Cc1nc(NC(=O)N[C@@H](COC(=O)[C@@H](N)C(C)C)c2ccc(-c3cccc4ncsc34)cc2)cs1 ; C#Cc1nc(NC(=O)N[C@@H](COC(=O)CCC/C=C\C/C=C\C/C=C\C/C=C\CCCCC)c2ccc(-c3cccc4ncsc34)cc2)cs1]

(R)-1-(1-(4-(Benzo[d]thiazol-7-yl)phenyl)-2-hydroxyethyl)-3-(2-ethynylthiazol-5-yl)urea A118. ¹H NMR (400 MHz, DMSO-d₆) δ 10.17 (s, 1H), 9.45 (s, 1H), 8.10 (dd, J=8.1, 1.0 Hz, 1H), 7.70 (t, J=5.6 Hz, 2H), 7.66 (d, J=8.0 Hz, 1H), 7.61-7.56 (m, 1H), 7.50 (d, J=8.2 Hz, 2H), 7.37 (s, 1H), 7.22 (d, J=7.8 Hz, 1H), 5.12 (t, J=5.2 Hz, 1H), 4.85 (dd, J=12.9, 5.5 Hz, 1H), 4.65 (s, 1H), 3.80-3.61 (m, 2H); MS (ESI) m/z: 421.1 [M+H⁺].

(R)-1-(1-(4-(Benzo[d]thiazol-7-yl)phenyl)-2-hydroxyethyl)-3-(2-ethynyl-5-methylthiazol-4-yl)urea A119. ¹H NMR (400 MHz, DMSO-d₆) δ 9.45 (s, 1H), 8.56 (s, 1H), 8.10 (dd, J=8.1, 1.0 Hz, 1H), 7.89-6.97 (m, 8H), 7.26 (s, 1H), 5.55-4.26 (m, 3H), 3.70 (dtd, J=16.4, 10.8, 5.3 Hz, 2H); MS (ESI) m/z: 435.1 [M+H⁺].

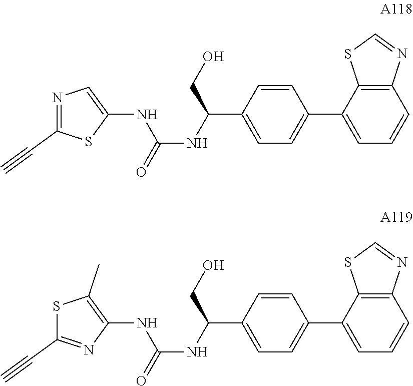 [SMILES: C#Cc1ncc(NC(=O)N[C@@H](CO)c2ccc(-c3cccc4ncsc34)cc2)s1 ; C#Cc1nc(NC(=O)N[C@@H](CO)c2ccc(-c3cccc4ncsc34)cc2)c(C)s1]

(R)-1-(1-(4-(Benzo[d]thiazol-7-yl)phenyl)-2-hydroxyethyl)-3-(5-ethynyl-1,3,4-thiadiazol-2-yl)urea A120. ¹H NMR (400 MHz, DMSO-d₆) δ 11.25 (s, 1H), 9.46 (s, 1H), 8.10 (dd, J=8.1, 1.0 Hz, 1H), 7.71 (d, J=8.3 Hz, 2H), 7.67 (t, J=7.8 Hz, 1H), 7.58 (d, J=6.8 Hz, 1H), 7.51 (d, J=8.2 Hz, 2H), 7.46 (d, J=7.1 Hz, 1H), 5.19 (t, J=4.9 Hz, 1H), 5.04 (s, 1H), 4.89 (dd, J=12.2, 4.9 Hz, 1H), 3.84-3.64 (m, 2H); MS (ESI) m/z: 422.1 [M+H⁺].

(R)-1-(1-(4-(Benzo[d]thiazol-7-yl)phenyl)-2-hydroxyethyl)-3-(3-ethynyl-1,2,4-thiadiazol-5-yl)urea A121. ¹H NMR (400 MHz, DMSO-d₆) δ 11.38 (s, 1H), 9.45 (s, 1H), 8.10 (dd, J=8.1, 1.1 Hz, 1H), 7.72 (d, J=8.3 Hz, 2H), 7.69-7.64 (m, 1H), 7.62 (d, J=7.9 Hz, 1H), 7.58 (d, J=6.8 Hz, 1H), 7.52 (d, J=8.2 Hz, 2H), 5.20 (t, J=5.0 Hz, 1H), 4.90 (d, J=6.8 Hz, 1H), 4.36 (s, 1H), 3.84-3.65 (m, 2H); MS (ESI) nm/z: 422.1 [M+H⁺].

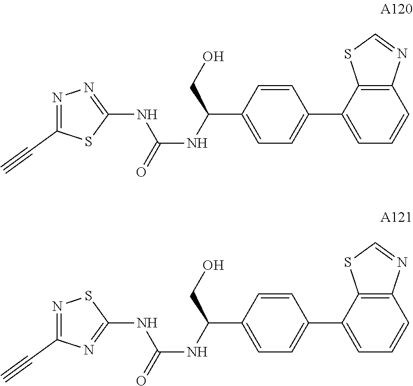 [SMILES: C#Cc1nnc(NC(=O)N[C@@H](CO)c2ccc(-c3cccc4ncsc34)cc2)s1 ; C#Cc1nsc(NC(=O)N[C@@H](CO)c2ccc(-c3cccc4ncsc34)cc2)n1]

(R)-1-(1-(4-(Benzo[d]thiazol-7-yl)phenyl)-2-hydroxyethyl)-3-(5-ethynyl-1,2,4-thiadiazol-3-yl)urea A122. ¹H NMR (400 MHz, DMSO-d₆) δ 10.69 (s, 1H), 9.45 (s, 1H), 8.70-8.51 (m, 1H), 8.13-8.05 (m, 1H), 7.68 (dd, J=17.1, 8.1 Hz, 3H), 7.58 (d, J=7.0 Hz, 1H), 7.51 (d, J=8.2 Hz, 2H), 5.67 (s, 1H), 5.15 (t, J=5.1 Hz, 1H), 4.96 (dd, J=12.3, 4.8 Hz, 1H), 3.74 (ddd, J=29.2, 10.9, 5.1 Hz, 2H); MS (ESI) m/z: 422.1 [M+H⁺].

(R)-1-(4-Ethynylpyrimidin-2-yl)-3-(2-hydroxy-1-(4-(6-(pyrrolidin-1-yl)pyridin-2-yl)phenyl)ethyl)urea A123. ¹H NMR (400 MHz, DMSO-d₆) δ 9.98 (s, 1H), 9.60 (d, J=7.9 Hz, 1H), 8.66 (d, J=5.1 Hz, 1H), 7.99 (d, J=8.4 Hz, 2H), 7.54 (dd, J=8.2, 7.5 Hz, 1H), 7.40 (d, J=8.3 Hz, 2H), 7.20 (d, J=5.0 Hz, 1H), 7.07 (d, J=7.3 Hz, 1H), 6.39 (d, J=8.3 Hz, 1H), 5.03 (t, J=5.3 Hz, 1H), 4.92 (dd, J=12.9, 5.2 Hz, 1H), 3.79-3.59 (m, 2H), 3.46 (t, J=6.5 Hz, 4H), 1.99-1.91 (m, 4H); MS (ESI) m/z: 429.1 [M+H⁺].

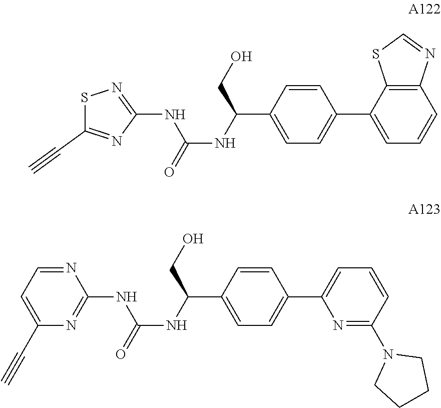 [SMILES: C#Cc1nc(NC(=O)N[C@@H](CO)c2ccc(-c3cccc4ncsc34)cc2)ns1 ; C#Cc1ccnc(NC(=O)N[C@@H](CO)c2ccc(-c3cccc(N4CCCC4)n3)cc2)n1]

(R)-1-(6-Ethynylpyridin-2-yl)-3-(2-hydroxy-1-(4-(6-(pyrrolidin-1-yl)pyridin-2-yl)phenyl)ethyl)urea A124. ¹H NMR (400 MHz, DMSO-d₆) δ 9.45 (s, 1H), 8.44 (s, 1H), 7.99 (d, J=8.3 Hz, 2H), 7.69 (dd, J=8.3, 7.5 Hz, 1H), 7.63-7.46 (m, 2H), 7.40 (d, J=8.3 Hz, 2H), 7.10 (dd, J=17.1, 7.3 Hz, 2H), 6.39 (d, J=8.3 Hz, 1H), 5.19-4.75 (m, 2H), 4.33 (s, 1H), 3.79-3.55 (m, 2H), 3.46 (t, J=6.4 Hz, 4H), 1.96 (t, J=6.5 Hz, 4H); MS (ESI) m/z: 428.2 [M+H⁺].

(R)-1-(2-Ethynylpyrimidin-4-yl)-3-(2-hydroxy-1-(4-(6-(pyrrolidin-1-yl)pyridin-2-yl)phenyl)ethyl)urea A125. ¹H NMR (400 MHz, DMSO-d₆) δ 9.82 (s, 1H), 8.45 (d, J=5.9 Hz, 1H), 7.96 (dd, J=27.1, 7.8 Hz, 3H), 7.64 (d, J=5.9 Hz, 1H), 7.59-7.48 (m, 1H), 7.38 (d, J=8.3 Hz, 2H), 7.08 (d, J=7.4 Hz, 1H), 6.39 (d, J=8.3 Hz, 1H), 5.06 (s, 1H), 4.83 (d, J=7.5 Hz, 1H), 4.31 (s, 1H), 3.84-3.53 (m, 2H), 3.46 (t, J=6.5 Hz, 4H), 2.06-1.89 (m, 4H); MS (ESI)

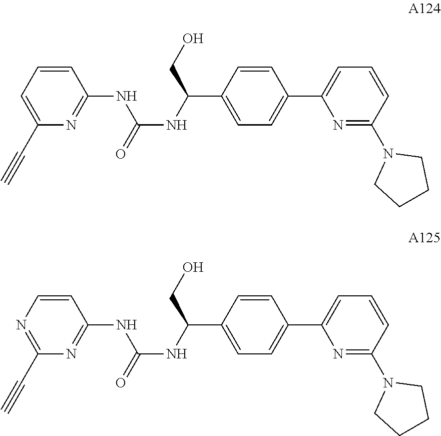 [SMILES: C#Cc1cccc(NC(=O)N[C@@H](CO)c2ccc(-c3cccc(N4CCCC4)n3)cc2)n1 ; C#Cc1nccc(NC(=O)N[C@@H](CO)c2ccc(-c3cccc(N4CCCC4)n3)cc2)n1]

(R)-1-(1-(4-(3,3-Difluoroazetidin-1-yl)phenyl)-2-hydroxyethyl)-3-(2-ethynyl-thiazol-4-yl)urea A126. ¹H NMR (400 MHz, DMSO-d₆) δ 9.36 (s, 1H), 7.25 (s, 1H), 7.03 (d, J=8.6 Hz, 2H), 6.79 (d, J=7.9 Hz, 1H), 6.65 (d, J=8.6 Hz, 2H), 5.92 (s, 1H), 4.88 (s, 1H), 4.60 (dd, J=13.2, 5.7 Hz, 1H), 4.07 (t, J=14.2 Hz, 2H), 3.64 (d, J=14.4 Hz, 2H), 3.53 (ddd, J=17.0, 10.8, 5.6 Hz, 3H); MS (ESI) m/z: 379.1 [M+H⁺].

(R)-2-(4-(3,3-Difluoroazetidin-1-yl)phenyl)-2-(3-(2-ethynylthiazol-4-yl)ureido)-ethyl carbamate A127. ¹H NMR (400 MHz, DMSO-d₆) δ 9.36 (s, 1H), 7.26 (d, J=6.8 Hz, 1H), 7.05 (t, J=12.1 Hz, 2H), 6.80 (d, J=8.3 Hz, 1H), 6.68 (d, J=8.6 Hz, 2H), 6.53 (s, 2H), 6.01 (t, J=6.8 Hz, 1H), 4.89 (s, 1H), 4.82 (dd, J=13.4, 7.8 Hz, 1H), 4.14-3.97 (m, 4H), 3.63 (td, J=14.2, 6.8 Hz, 2H); MS (ESI) m/z: 291.1 [Fragment+H⁺].

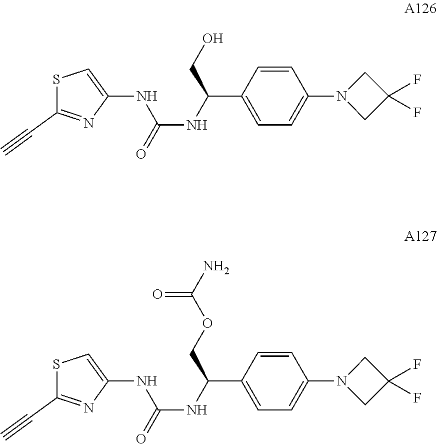 [SMILES: C#Cc1nc(NC(=O)N[C@@H](CO)c2ccc(N3CC(F)(F)C3)cc2)cs1 ; C#Cc1nc(NC(=O)N[C@@H](COC(N)=O)c2ccc(N3CC(F)(F)C3)cc2)cs1]

(R)-1-(2-Ethynylthiazol-4-yl)-3-(2-hydroxy-1-(4-(pyrrolidin-1-yl)phenyl)ethyl)-urea A128. ¹H NMR (400 MHz, DMSO-d₆) δ 9.36 (s, 1H), 7.25 (s, 1H), 7.08 (d, J=8.6 Hz, 2H), 6.79 (d, J=8.0 Hz, 1H), 6.49 (d, J=8.6 Hz, 2H), 4.88 (s, 1H), 4.86 (t, J=5.2 Hz, 1H), 4.62 (dd, J=13.0, 6.0 Hz, 1H), 3.57 (dt, J=10.1, 4.9 Hz, 1H), 3.53-3.45 (m, 1H), 3.18 (t, J=6.5 Hz, 4H), 2.03-1.85 (m, 4H).

(R)-1-(2-Ethynylthiazol-4-yl)-3-(2-hydroxy-1-(4-(2-oxopyrrolidin-1-yl)phenyl)-ethyl)urea A129. ¹H NMR (400 MHz, DMSO-d₆) δ 9.45 (s, 1H), 7.58 (d, J=8.7 Hz, 2H), 7.29 (d, J=8.6 Hz, 2H), 7.25 (s, 1H), 6.96 (d, J=7.8 Hz, 1H), 4.99 (t, J=5.2 Hz, 1H), 4.89 (s, 1H), 4.72 (dd, J=12.9, 5.4 Hz, 1H), 3.81 (t, J=7.1 Hz, 2H), 3.64 (dt, J=10.0, 5.0 Hz, 1H), 3.55 (dt, J=11.0, 5.6 Hz, 1H), 2.52 (d, J=1.9 Hz, 2H), 2.09-1.98 (m, 2H); MS (ESI) m/z: 371.1 [M+H⁺].

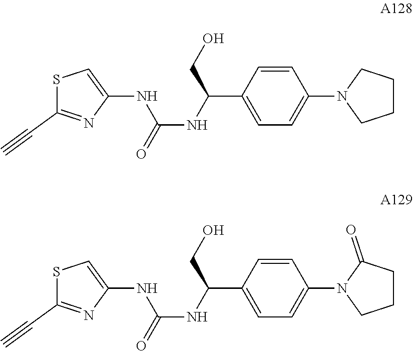 [SMILES: C#Cc1nc(NC(=O)N[C@@H](CO)c2ccc(N3CCCC3)cc2)cs1 ; C#Cc1nc(NC(=O)N[C@@H](CO)c2ccc(N3CCCC3=O)cc2)cs1]

(R)-1-(1-(4-(3,3-Difluoropyrrolidin-1-yl)phenyl)-2-hydroxyethyl)-3-(2-ethynyl-thiazol-4-yl)urea A130. ¹H NMR (400 MHz, DMSO-d₆) δ 9.38 (s, 1H), 7.25 (s, 1H), 7.14 (d, J=8.6 Hz, 2H), 6.84 (d, J=7.9 Hz, 1H), 6.58 (d, J=8.7 Hz, 2H), 4.91 (d, J=5.2 Hz, 1H), 4.89 (s, 1H), 4.73-4.54 (m, 1H), 3.65 (t, J=13.5 Hz, 2H), 3.58 (dd, J=10.6, 5.3 Hz, 1H), 3.55-3.47 (m, 1H), 3.43 (t, J=7.2 Hz, 2H), 2.58-2.51 (m, 2H).

(R)-1-(2-Ethynylthiazol-4-yl)-3-(2-hydroxy-1-(4-(2-oxopiperidin-1-yl)phenyl)-ethyl)urea A131. ¹H NMR (400 MHz, DMSO-d₆) δ 9.46 (s, 1H), 7.30 (d, J=8.4 Hz, 2H), 7.26 (s, 1H), 7.23-7.17 (m, 2H), 7.00 (d, J=7.8 Hz, 1H), 5.05 (t, J=5.1 Hz, 1H), 4.89 (s, 1H), 4.75 (dd, J=12.8, 5.3 Hz, 1H), 3.66 (dt, J=9.9, 4.9 Hz, 1H), 3.58 (dt, J=11.0, 5.7 Hz, 3H), 2.37 (t, J=6.3 Hz, 2H), 1.89-1.77 (m, 4H); MS (ESI) m/z: 385.2 [M+H⁺].

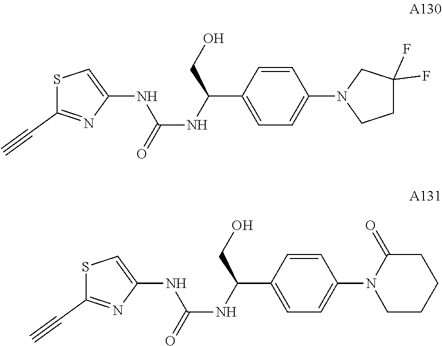 [SMILES: C#Cc1nc(NC(=O)N[C@@H](CO)c2ccc(N3CCC(F)(F)C3)cc2)cs1 ; C#Cc1nc(NC(=O)N[C@@H](CO)c2ccc(N3CCCCC3=O)cc2)cs1]

(R)-1-(2-Ethynylthiazol-4-yl)-3-(2-hydroxy-1-(4-(2-oxopyridin-1(2H)-yl)phenyl)-ethyl)urea A132. ¹H NMR (400 MHz, DMSO-d₆) δ 9.51 (s, 1H), 7.62 (dd, J=6.9, 1.5 Hz, 1H), 7.53-7.46 (m, 1H), 7.43 (d, J=8.4 Hz, 2H), 7.37-7.32 (m, 2H), 7.27 (s, 1H), 7.09 (d, J=7.8 Hz, 1H), 6.49-6.44 (m, 1H), 6.30 (td, J=6.7, 1.3 Hz, 1H), 5.11 (t, J=5.0 Hz, 1H), 4.89 (s, 1H), 4.82 (dd, J=12.6, 5.1 Hz, 1H), 3.71 (dt, J=9.8, 4.8 Hz, 1H), 3.63 (dt, J=10.7, 5.4 Hz, 1H); MS (ESI) m/z: 381.2 [M+H⁺].

(R)-1-(2-Ethynylthiazol-4-yl)-3-(2-hydroxy-1-(4-morpholinophenyl)ethyl)urea A133. ¹H NMR (400 MHz, DMSO-d₆) δ 9.40 (s, 1H), 7.25 (s, 1H), 7.16 (d, J=8.7 Hz, 2H), 6.93-6.84 (m, 3H), 4.93 (t, J=5.2 Hz, 1H), 4.89 (s, 1H), 4.66 (dd, J=13.1, 5.6 Hz, 1H), 3.78-3.67 (m, 4H), 3.56 (ddt, J=22.2, 11.0, 5.4 Hz, 2H), 3.12-2.99 (m, 4H).

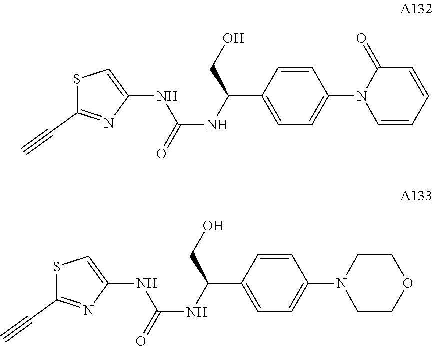 [SMILES: C#Cc1nc(NC(=O)N[C@@H](CO)c2ccc(-n3ccccc3=O)cc2)cs1 ; C#Cc1nc(NC(=O)N[C@@H](CO)c2ccc(N3CCOCC3)cc2)cs1]

(R)-1-(2-Ethynylthiazol-4-yl)-3-(2-hydroxy-1-(4-(3-oxomorpholino)phenyl)-ethyl)urea A134. ¹H NMR (400 MHz, DMSO-d₆) δ 9.49 (s, 1H), 7.33 (s, 4H), 7.26 (s, 1H), 7.04 (d, J=4.8 Hz, 1H), 5.06 (t, J=4.9 Hz, 1H), 4.89 (s, 1H), 4.76 (dd, J=12.8, 5.3 Hz, 1H), 4.19 (s, 2H), 3.99-3.94 (m, 2H), 3.70 (dd, J=9.6, 4.7 Hz, 2H), 3.61 (ddd, J=16.1, 10.2, 5.2 Hz, 2H); MS (ESI) m/z: 387.1 [M+H⁺].

(R)-1-(2-Ethynylthiazol-4-yl)-3-(2-hydroxy-1-(5-(piperidin-1-yl)pyridin-2-yl)-ethyl)urea A135. ¹H NMR (400 MHz, DMSO-d₆) δ 9.56 (s, 1H), 8.23 (d, J=2.8 Hz, 1H), 7.29 (dd, J=8.6, 2.9 Hz, 1H), 7.27 (s, 1H), 7.14 (d, J=8.6 Hz, 1H), 6.97 (d, J=8.1 Hz, 1H), 4.89 (s, 1H), 4.85 (t, J=5.3 Hz, 1H), 4.73 (dt, J=8.0, 5.6 Hz, 1H), 3.65 (dd, J=10.3, 5.2 Hz, 1H), 3.60 (dd, J=10.6, 5.3 Hz, 1H), 3.24-3.08 (m, 4H), 1.68-1.58 (m, 4H), 1.58-1.49 (m, 2H); MS (ESI) m/z: 372.2 [M+H⁺].

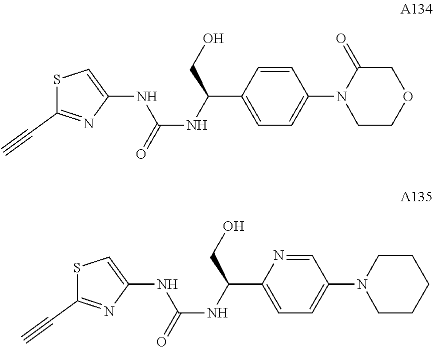 [SMILES: C#Cc1nc(NC(=O)N[C@@H](CO)c2ccc(N3CCOCC3=O)cc2)cs1 ; C#Cc1nc(NC(=O)N[C@@H](CO)c2ccc(N3CCCCC3)cn2)cs1]

(R)-1-(1-(5-(3,3-Difluoropiperidin-1-yl)pyridin-2-yl)-2-hydroxyethyl)-3-(2-ethynylthiazol-4-yl)urea A136. ¹H NMR (400 MHz, DMSO-d₆) δ 9.57 (s, 1H), 8.28 (d, J=2.8 Hz, 1H), 7.37 (dd, J=8.7, 3.0 Hz, 1H), 7.27 (s, 1H), 7.16 (d, J=8.6 Hz, 1H), 6.99 (d, J=8.1 Hz, 1H), 4.89 (s, 2H), 4.73 (dd, J=13.4, 5.4 Hz, 1H), 3.63 (dd, J=14.5, 5.9 Hz, 2H), 3.52 (t, J=11.9 Hz, 2H), 3.29-3.25 (m, 2H), 2.13-1.92 (m, 3H), 1.80 (d, J=11.8 Hz, 2H); MS (ESI) m/z: 408.1 [M+H⁺].

(R)-1-(1-(4-(Azepan-1-yl)phenyl)-2-hydroxyethyl)-3-(2-ethynylthiazol-4-yl)urea A137. ¹H NMR (400 MHz, DMSO-d₆) δ 9.36 (s, 1H), 7.25 (s, 1H), 7.06 (d, J=8.7 Hz, 2H), 6.78 (d, J=8.0 Hz, 1H), 6.61 (d, J=8.8 Hz, 2H), 4.90-4.83 (m, 2H), 4.60 (dd, J=13.3, 5.8 Hz, 1H), 3.57 (dq, J=10.1, 5.1 Hz, 1H), 3.55-3.46 (m, 1H), 3.41 (t, J=6.0 Hz, 4H), 1.70 (s, 4H), 1.52-1.38 (m, 4H); MS (ESI) m/z: 281.2 [Fragment+H⁺].

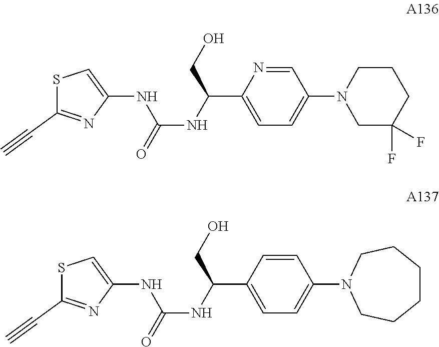 [SMILES: C#Cc1nc(NC(=O)N[C@@H](CO)c2ccc(N3CCCC(F)(F)C3)cn2)cs1 ; C#Cc1nc(NC(=O)N[C@@H](CO)c2ccc(N3CCCCCC3)cc2)cs1]

(R)-1-(2-Ethynylthiazol-4-yl)-3-(2-hydroxy-1-(4-(2-oxoazepan-1-yl)phenyl)-ethyl)urea A138. ¹H NMR (400 MHz, DMSO-d₆) δ 9.46 (s, 1H), 7.28 (d, J=8.4 Hz, 2H), 7.25 (s, 1H), 7.14 (d, J=8.4 Hz, 2H), 6.99 (d, J=7.7 Hz, 1H), 5.04 (t, J=5.1 Hz, 1H), 4.89 (s, 1H), 4.73 (dd, J=12.8, 5.3 Hz, 1H), 3.70 (d, J=8.1 Hz, 2H), 3.65 (dd, J=10.4, 5.3 Hz, 1H), 3.57 (dt, J=10.8, 5.5 Hz, 1H), 2.58 (d, J=10.3 Hz, 2H), 1.71 (d, J=7.1 Hz, 6H); MS (ESI) m/z: 399.2 [M+H⁺].

(R)-1-(2-Ethynylthiazol-4-yl)-3-(2-hydroxy-1-(4-(1-methyl-1,5,6,7-tetrahydro-4H-pyrazolo[4,3-b]pyridin-4-yl)phenyl)ethyl)urea A139. ¹H NMR (400 MHz, DMSO-d₆) δ 9.41 (s, 1H), 7.25 (d, J=3.8 Hz, 2H), 7.17 (d, J=8.6 Hz, 2H), 7.05 (d, J=8.7 Hz, 2H), 6.89 (d, J=7.9 Hz, 1H), 4.95 (t, J=5.2 Hz, 1H), 4.89 (s, 1H), 4.67 (dd, J=13.0, 5.6 Hz, 1H), 3.67 (s, 3H), 3.58 (ddt, J=22.2, 11.0, 5.4 Hz, 2H), 3.50-3.43 (m, 2H), 2.69 (t, J=6.6 Hz, 2H), 1.94-1.84 (m, 2H); MS (ESI) m/z: 256.1 [Fragment+H⁺].

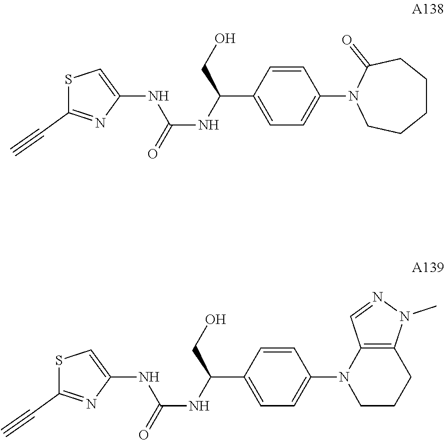 [SMILES: C#Cc1nc(NC(=O)N[C@@H](CO)c2ccc(N3CCCCCC3=O)cc2)cs1 ; C#Cc1nc(NC(=O)N[C@@H](CO)c2ccc(N3CCCc4c3cnn4C)cc2)cs1]

(R)-1-(2-Ethynylthiazol-4-yl)-3-(2-hydroxy-1-(4-(2-methyl-2,5,6,7-tetrahydro-4H-pyrazolo[4,3-b]pyridin-4-yl)phenyl)ethyl)urea A140. ¹H NMR (400 MHz, DMSO-d₆) δ 9.41 (s, 1H), 7.25 (d, J=3.8 Hz, 2H), 7.17 (d, J=8.6 Hz, 2H), 7.05 (d, J=8.7 Hz, 2H), 6.89 (d, J=7.9 Hz, 1H), 4.95 (t, J=5.2 Hz, 1H), 4.89 (s, 1H), 4.67 (dd, J=13.0, 5.6 Hz, 1H), 3.67 (s, 3H), 3.58 (ddt, J=22.2, 11.0, 5.4 Hz, 2H), 3.50-3.43 (m, 2H), 2.69 (t, J=6.6 Hz, 2H), 1.94-1.84 (m, 2H); MS (ESI) m/z: 256.2 [Fragment+H⁺].

(R)-1-(1-(6′-Cyano-2′,3′,4′,5′-tetrahydro-[1,1′-biphenyl]-4-yl)-2-hydroxyethyl)-3-(2-ethynylthiazol-4-yl)urea A141. ¹H NMR (400 MHz, DMSO-d₆) δ 9.49 (s, 1H), 7.38 (q, J=8.2 Hz, 4H), 7.27 (s, 1H), 7.05 (d, J=7.8 Hz, 1H), 5.07 (s, 1H), 4.88 (s, 1H), 4.80 (dd, J=12.1, 5.0 Hz, 1H), 3.66 (d, J=19.7 Hz, 2H), 2.47 (s, 2H), 2.36 (s, 2H), 1.69 (s, 4H); MS (ESI) m/z: 393.1 [M+H⁺].

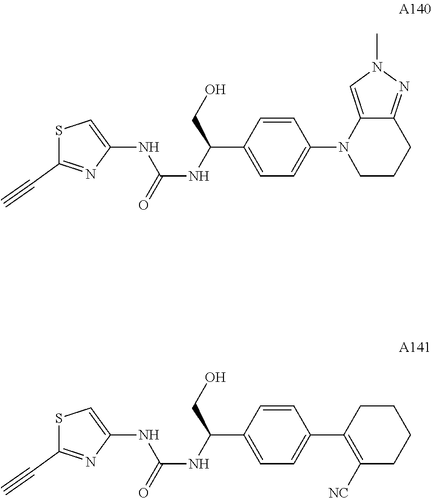 [SMILES: C#Cc1nc(NC(=O)N[C@@H](CO)c2ccc(N3CCCc4nn(C)cc43)cc2)cs1 ; C#Cc1nc(NC(=O)N[C@@H](CO)c2ccc(C3=C(C#N)CCCC3)cc2)cs1]

(R)-1-(1-(5-(2-Cyanocyclohex-1-en-1-yl)pyridin-2-yl)-2-hydroxyethyl)-3-(2-ethynylthiazol-4-yl)urea A142. ¹H NMR (400 MHz, DMSO-d₆) δ 9.63 (s, 1H), 8.62 (d, J=2.1 Hz, 1H), 7.84 (dd, J=8.1, 2.3 Hz, 1H), 7.41 (d, J=8.1 Hz, 1H), 7.28 (s, 1H), 7.14 (d, J=8.0 Hz, 1H), 5.02 (t, J=5.2 Hz, 1H), 4.89 (s, 1H), 4.88-4.83 (m, 1H), 3.73 (ddt, J=21.3, 10.5, 5.2 Hz, 2H), 2.39 (d, J=5.9 Hz, 2H), 1.77-1.63 (m, 4H); MS (ESI) m/z: 394.1 [M+H⁺].

1-((1R)-1-(4-(1-Acetylpiperidin-2-yl)phenyl)-2-hydroxyethyl)-3-(2-ethynyl-thiazol-4-yl)urea A143. ¹H NMR (400 MHz, DMSO-d₆) δ 9.46 (s, 1H), 7.35-7.26 (m, 2H), 7.25 (s, 1H), 7.15 (d, J=7.5 Hz, 2H), 6.99 (d, J=7.9 Hz, 1H), 5.00 (t, J=5.0 Hz, 1H), 4.89 (s, 1H), 4.74 (d, J=6.2 Hz, 1H), 3.61 (ddt, J=34.6, 10.9, 5.3 Hz, 2H), 2.53-2.51 (m, 3H), 2.36-2.23 (m, 1H), 2.17-1.94 (m, 3H), 1.55 (d, J=10.4 Hz, 2H), 1.35 (d, J=7.5 Hz, 1H), 1.25 (dd, J=14.2, 4.8 Hz, 2H); MS (ESI) m/z: 413.1 [M+H⁺].

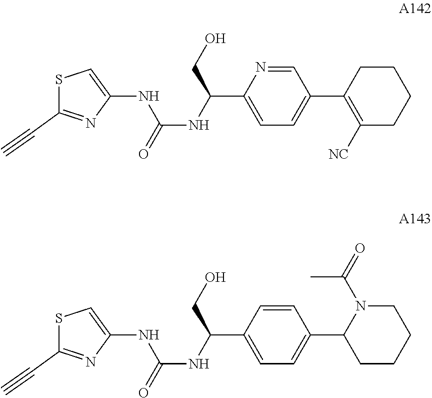 [SMILES: C#Cc1nc(NC(=O)N[C@@H](CO)c2ccc(C3=C(C#N)CCCC3)cn2)cs1 ; C#Cc1nc(NC(=O)N[C@@H](CO)c2ccc(C3CCCCN3C(C)=O)cc2)cs1]

(R)-1-(1-(3-(2-Cyanophenyl)azetidin-1-yl)-3-hydroxy-1-oxopropan-2-yl)-3-(2-ethynylthiazol-4-yl)urea A144. ¹H NMR (400 MHz, DMSO-d₆) δ 9.55 (s, 1H), 7.83 (d, J=7.6 Hz, 1H), 7.75 (dd, J=10.9, 3.0 Hz, 2H), 7.52-7.45 (m, 1H), 7.31 (d, J=8.6 Hz, 1H), 6.80 (d, J=7.7 Hz, 1H), 5.15 (t, J=5.6 Hz, 1H), 4.90 (s, 1H), 4.77 (dd, J=20.0, 8.8 Hz, 1H), 4.46-4.27 (m, 3H), 4.21 (dq, J=8.9, 6.2 Hz, 1H), 4.00 (ddd, J=23.4, 9.6, 6.1 Hz, 1H), 3.55 (ddd, J=19.0, 9.4, 4.9 Hz, 2H); MS (ESI) m/z: 396.1 [M+H⁺].

(S)-1-(1-(3-(2-Cyanophenyl)azetidin-1-yl)-3-hydroxy-1-oxopropan-2-yl)-3-(2-ethynylthiazol-4-yl)urea A145. ¹H NMR (400 MHz, DMSO-d₆) δ 9.54 (s, 1H), 7.83 (d, J=7.6 Hz, 1H), 7.76 (d, J=4.7 Hz, 2H), 7.53-7.44 (m, 1H), 7.30 (t, J=6.1 Hz, 1H), 6.80 (d, J=7.7 Hz, 1H), 5.15 (t, J=5.6 Hz, 1H), 4.90 (s, 1H), 4.77 (dd, J=19.9, 8.8 Hz, 1H), 4.47-4.28 (m, 3H), 4.27-4.16 (m, 1H), 4.00 (ddd, J=23.3, 9.6, 6.1 Hz, 1H), 3.65-3.48 (m, 2H); MS (ESI) m/z: 396.1 [M+H⁺].

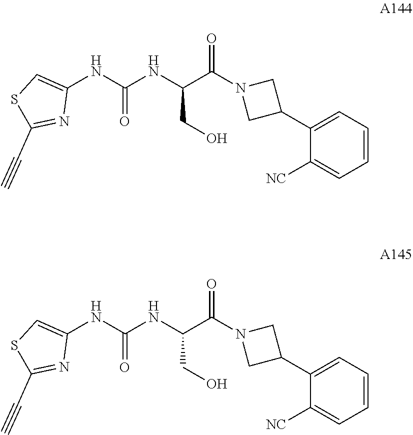 [SMILES: C#Cc1nc(NC(=O)N[C@H](CO)C(=O)N2CC(c3ccccc3C#N)C2)cs1 ; C#Cc1nc(NC(=O)N[C@@H](CO)C(=O)N2CC(c3ccccc3C#N)C2)cs1]

(R)-1-(1-(4-(2-Cyanophenyl)piperidin-1-yl)-3-hydroxy-1-oxopropan-2-yl)-3-(2-ethynylthiazol-4-yl)urea A146. ¹H NMR (400 MHz, DMSO-d₆) δ 9.59 (d, J=13.6 Hz, 1H), 7.81 (d, J=7.7 Hz, 1H), 7.68 (dd, J=16.5, 7.8 Hz, 1H), 7.54-7.47 (m, 1H), 7.43 (t, J=6.8 Hz, 1H), 7.30 (s, 1H), 6.89-6.74 (m, 1H), 5.04 (dt, J=11.4, 5.2 Hz, 1H), 4.90 (s, 1H), 4.84 (dd, J=13.5, 6.3 Hz, 1H), 4.61 (d, J=12.7 Hz, 1H), 4.21 (d, J=12.8 Hz, 1H), 3.55 (t, J=5.6 Hz, 2H), 3.27-3.08 (m, 2H), 2.74 (t, J=14.7 Hz, 1H), 1.85 (t, J=12.5 Hz, 2H), 1.76-1.65 (m, 1H), 1.57 (d, J=12.1 Hz, 1H); MS (ESI) m/z: 424.2 [M+H⁺].

(S)-1-(1-(4-(2-Cyanophenyl)piperidin-1-yl)-3-hydroxy-1-oxopropan-2-yl)-3-(2-ethynylthiazol-4-yl)urea A147. ¹H NMR (400 MHz, DMSO-d₆) δ 9.60 (d, J=13.8 Hz, 1H), 7.81 (d, J=7.6 Hz, 1H), 7.71-7.62 (m, 1H), 7.56-7.38 (m, 2H), 7.30 (s, 1H), 6.88-6.75 (m, 1H), 5.04 (d, J=36.1 Hz, 1H), 4.90 (s, 1H), 4.84 (d, J=7.3 Hz, 1H), 4.60 (d, J=10.0 Hz, 1H), 4.21 (d, J=12.4 Hz, 1H), 3.55 (t, J=5.5 Hz, 2H), 3.18 (dd, J=26.8, 13.9 Hz, 2H), 2.69 (dd, J=14.2, 12.4 Hz, 1H), 1.83 (d, J=11.5 Hz, 2H), 1.78-1.49 (m, 2H); MS (ESI) m/z: 424.1 [M+H⁺].

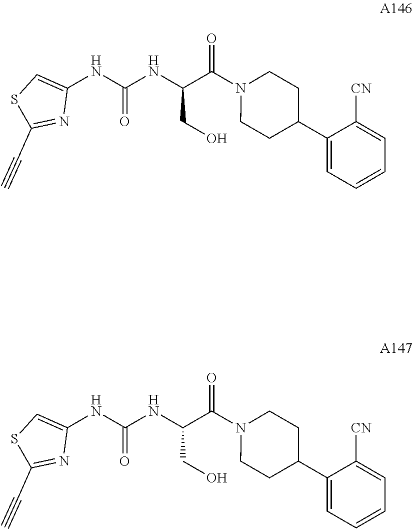 [SMILES: C#Cc1nc(NC(=O)N[C@H](CO)C(=O)N2CCC(c3ccccc3C#N)CC2)cs1 ; C#Cc1nc(NC(=O)N[C@@H](CO)C(=O)N2CCC(c3ccccc3C#N)CC2)cs1]

(R)-1-(1-(4-(2-Cyanophenyl)piperazin-1-yl)-3-hydroxy-1-oxopropan-2-yl)-3-(2-ethynylthiazol-4-yl)urea A148. ¹H NMR (400 MHz, DMSO-d₆) δ 9.60 (s, 1H), 7.74 (dd, J=7.7, 1.6 Hz, 1H), 7.62 (ddd, J=8.4, 7.5, 1.6 Hz, 1H), 7.30 (s, 1H), 7.23-7.08 (m, 2H), 6.82 (d, J=8.2 Hz, 1H), 5.05 (t, J=5.8 Hz, 1H), 4.90 (s, 1H), 4.87-4.78 (m, 1H), 3.73 (d, J=30.7 Hz, 4H), 3.63-3.50 (m, 2H), 3.16 (d, J=22.0 Hz, 4H); MS (ESI) m/z: 425.2 [M+H⁺].

(S)-1-(1-(4-(2-Cyanophenyl)piperazin-1-yl)-3-hydroxy-1-oxopropan-2-yl)-3-(2-ethynylthiazol-4-yl)urea A149. ¹H NMR (400 MHz, DMSO-d₆) δ 9.60 (s, 1H), 7.74 (dd, J=7.7, 1.6 Hz, 1H), 7.66-7.55 (m, 1H), 7.30 (s, 1H), 7.19 (d, J=8.2 Hz, 1H), 7.14 (td, J=7.6, 0.7 Hz, 1H), 6.82 (d, J=8.2 Hz, 1H), 5.06 (t, J=5.8 Hz, 1H), 4.90 (s, 1H), 4.84 (dd, J=13.9, 5.9 Hz, 1H), 3.73 (d, J=30.5 Hz, 4H), 3.62-3.47 (m, 2H), 3.25-3.09 (m, 4H); MS (ESI) m/z: 425.2 [M+H⁺].

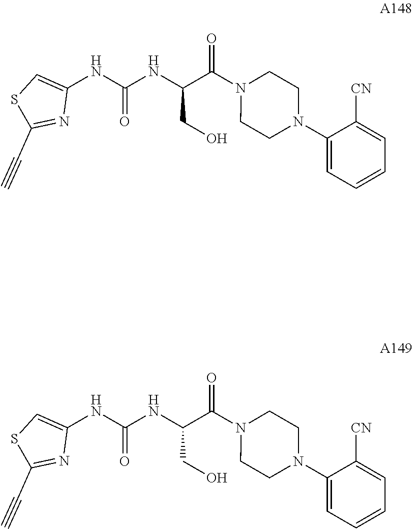 [SMILES: C#Cc1nc(NC(=O)N[C@H](CO)C(=O)N2CCN(c3ccccc3C#N)CC2)cs1 ; C#Cc1nc(NC(=O)N[C@@H](CO)C(=O)N2CCN(c3ccccc3C#N)CC2)cs1]

(R)-1-(1-(1-(2-Cyanophenyl)piperidin-4-yl)-2-hydroxyethyl)-3-(2-ethynylthiazol-4-yl)urea A150. ¹H NMR (400 MHz, DMSO-d₆) δ 9.35 (s, 1H), 7.67 (dd, J=7.6, 1.4 Hz, 1H), 7.60-7.54 (m, 1H), 7.29 (s, 1H), 7.15 (d, J=8.3 Hz, 1H), 7.06 (t, J=7.4 Hz, 1H), 6.40 (d, J=9.0 Hz, 1H), 4.88 (s, 1H), 4.81 (s, 1H), 3.54 (dd, J=23.3, 8.6 Hz, 4H), 3.42 (d, J=5.3 Hz, 1H), 2.80-2.71 (m, 2H), 1.84-1.67 (m, 3H), 1.45 (dt, J=20.9, 8.6 Hz, 2H); MS (ESI) m/z: 396.1 [M+H⁺].

(S)-1-(1-(1-(2-Cyanophenyl)piperidin-4-yl)-2-hydroxyethyl)-3-(2-ethynylthiazol-4-yl)urea A151. ¹H NMR (400 MHz, DMSO-d₆) δ 9.34 (s, 1H), 7.67 (dd, J=7.7, 1.6 Hz, 1H), 7.60-7.50 (m, 1H), 7.30 (s, 1H), 7.15 (d, J=8.3 Hz, 1H), 7.05 (t, J=7.5 Hz, 1H), 6.39 (d, J=9.0 Hz, 1H), 4.89 (s, 1H), 4.81 (t, J=5.0 Hz, 1H), 3.63-3.46 (m, 4H), 3.42 (dt, J=10.6, 5.1 Hz, 1H), 2.83-2.63 (m, 2H), 1.73 (ddd, J=20.5, 18.1, 12.3 Hz, 3H), 1.53-1.31 (m, 2H); MS (ESI) m/z: 396.1 [M+H⁺].

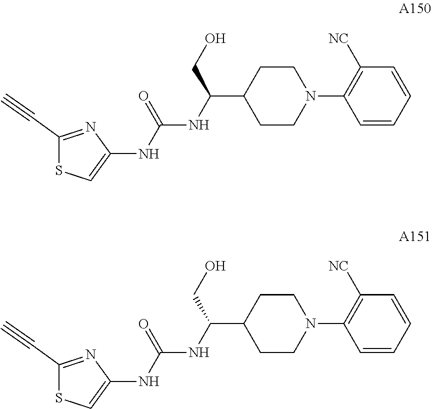 [SMILES: C#Cc1nc(NC(=O)N[C@@H](CO)C2CCN(c3ccccc3C#N)CC2)cs1 ; C#Cc1nc(NC(=O)N[C@H](CO)C2CCN(c3ccccc3C#N)CC2)cs1]

(S)-1-(2-Ethynylthiazol-4-yl)-3-(3-hydroxy-1-oxo-1-(6-azaspiro[2.5]octan-6-yl)propan-2-yl)urea A152. ¹H NMR (400 MHz, DMSO-d₆) δ 9.57 (s, 1H), 7.29 (s, 1H), 6.78 (d, J=8.3 Hz, 1H), 4.99 (t, J=5.8 Hz, 1H), 4.89 (s, 1H), 4.80 (dt, J=8.4, 5.9 Hz, 1H), 3.60-3.45 (m, 6H), 1.36 (d, J=5.7 Hz, 2H), 1.28 (d, J=5.8 Hz, 2H), 0.34 (d, J=4.1 Hz, 4H); MS (ESI) m/z: 396.1 [M+H⁺].

(S)-1-(2-Ethynylthiazol-4-yl)-3-(3-hydroxy-1-oxo-1-(7-azaspiro[3.5]nonan-7-yl)propan-2-yl)urea A153. ¹H NMR (400 MHz, DMSO-d₆) δ 9.56 (s, 1H), 7.28 (s, 1H), 6.76 (d, J=8.3 Hz, 1H), 4.97 (t, J=5.8 Hz, 1H), 4.89 (s, 1H), 4.78 (dt, J=8.1, 5.9 Hz, 1H), 3.62-3.40 (m, 6H), 1.59 (t, J=6.9 Hz, 4H), 1.38 (d, J=32.7 Hz, 6H); MS (ESI) m/z: 363.2 [M+H⁺].

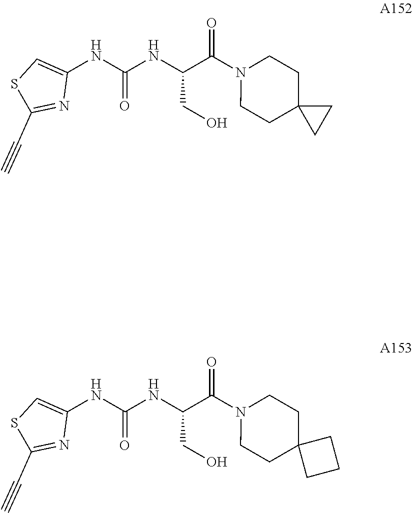 [SMILES: C#Cc1nc(NC(=O)N[C@@H](CO)C(=O)N2CCC3(CC2)CC3)cs1 ; C#Cc1nc(NC(=O)N[C@@H](CO)C(=O)N2CCC3(CCC3)CC2)cs1]

(S)-1-(2-Ethynylthiazol-4-yl)-3-(3-hydroxy-1-oxo-1-(8-azaspiro[4.5]decan-8-yl)propan-2-yl)urea A154. ¹H NMR (400 MHz, DMSO-d₆) δ 9.56 (s, 1H), 7.28 (s, 1H), 6.76 (d, J=8.3 Hz, 1H), 4.89 (s, 1H), 4.78 (dt, J=8.2, 5.9 Hz, 1H), 3.54-3.43 (m, 6H), 1.59 (t, J=6.9 Hz, 4H), 1.42 (s, 6H), 1.34 (s, 2H); MS (ESI) m/z: 377.2 [M+H⁺].

(R)-1-(2-Ethynylthiazol-4-yl)-3-(3-hydroxy-1-oxo-1-(8-azaspiro[4.5]decan-8-yl)propan-2-yl)urea A155. ¹H NMR (400 MHz, DMSO-d₆) δ 9.56 (s, 1H), 7.28 (s, 1H), 6.76 (d, J=8.3 Hz, 1H), 4.97 (t, J=5.8 Hz, 1H), 4.89 (s, 1H), 4.78 (dt, J=8.1, 5.9 Hz, 1H), 3.62-3.40 (m, 6H), 1.59 (t, J=6.9 Hz, 4H), 1.38 (d, J=32.7 Hz, 6H); MS (ESI) m/z: 377.1 [M+H⁺].

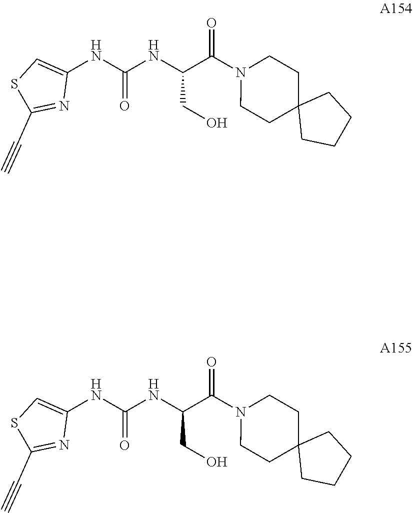 [SMILES: C#Cc1nc(NC(=O)N[C@@H](CO)C(=O)N2CCC3(CCCC3)CC2)cs1 ; C#Cc1nc(NC(=O)N[C@H](CO)C(=O)N2CCC3(CCCC3)CC2)cs1]

(S)-1-(2-Ethynylthiazol-5-yl)-3-(3-hydroxy-1-oxo-1-(1-oxo-8-azaspiro[4.5]decan-8-yl)propan-2-yl)urea A156. ¹H NMR (400 MHz, DMSO-d₆) δ 9.57 (s, 1H), 7.28 (d, J=3.8 Hz, 1H), 6.77 (d, J=7.5 Hz, 1H), 4.99 (d, J=24.7 Hz, 1H), 4.89 (s, 1H), 4.77 (dd, J=13.7, 6.0 Hz, 1H), 3.97 (dd, J=72.4, 12.8 Hz, 2H), 3.50 (t, J=5.6 Hz, 2H), 3.04 (dd, J=20.2, 13.0 Hz, 2H), 2.26 (t, J=7.4 Hz, 2H), 1.97-1.79 (m, 4H), 1.26 (dt, J=14.5, 11.1 Hz, 6H); MS (ESI) m/z: 391.1 [M+H⁺].

(S)-1-(2-Ethynylthiazol-4-yl)-3-(3-hydroxy-1-oxo-1-(3-azaspiro[5.5]undecan-3-yl)propan-2-yl)urea A157. ¹H NMR (400 MHz, DMSO-d₆) δ 9.56 (s, 1H), 7.28 (s, 1H), 6.76 (d, J=8.3 Hz, 1H), 4.97 (t, J=5.8 Hz, 1H), 4.89 (s, 1H), 4.77 (dt, J=8.2, 5.9 Hz, 1H), 3.48 (dd, J=10.1, 4.6 Hz, 6H), 1.36 (d, J=21.0 Hz, 14H); MS (ESI) m/z: 391.2 [M+H⁺].

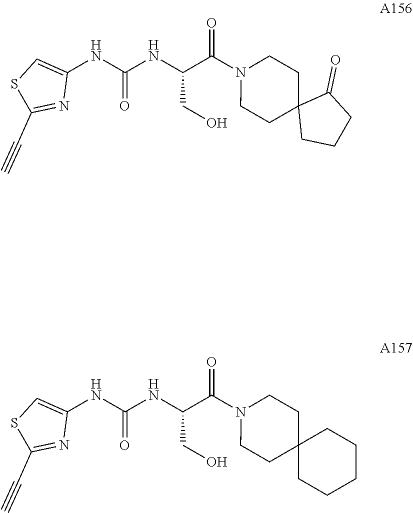 [SMILES: C#Cc1nc(NC(=O)N[C@@H](CO)C(=O)N2CCC3(CCCC3=O)CC2)cs1 ; C#Cc1nc(NC(=O)N[C@@H](CO)C(=O)N2CCC3(CCCCC3)CC2)cs1]

(S)-1-(1-(9,9-Difluoro-3-azaspiro[5.5]undecan-3-yl)-3-hydroxy-1-oxopropan-2-yl)-3-(2-ethynylthiazol-4-yl)urea A158. ¹H NMR (400 MHz, DMSO-d₆) δ 9.56 (s, 1H), 7.28 (s, 1H), 6.76 (d, J=8.3 Hz, 1H), 4.99 (t, J=5.8 Hz, 1H), 4.89 (s, 1H), 4.78 (dd, J=14.1, 5.9 Hz, 1H), 3.70-3.37 (m, 6H), 2.22-1.77 (m, 4H), 1.66-1.13 (m, 8H); MS (ESI) m/z: 427.1 [M+H⁺].

(S)-1-(1-(4-(3,3-Difluoroazetidin-1-yl)piperidin-1-yl)-3-hydroxy-1-oxopropan-2-yl)-3-(2-ethynylthiazol-4-yl)urea A159. ¹H NMR (400 MHz, DMSO-d₆) δ 9.56 (s, 1H), 7.28 (s, 1H), 6.76 (d, J=8.3 Hz, 1H), 4.98 (d, J=6.1 Hz, 1H), 4.89 (s, 1H), 4.79 (d, J=24.6 Hz, 1H), 3.99 (d, J=11.9 Hz, 1H), 3.85 (d, J=11.0 Hz, 1H), 3.55 (dd, J=22.3, 9.9 Hz, 4H), 3.50 (t, J=5.3 Hz, 2H), 3.20 (t, J=11.1 Hz, 1H), 2.96 (t, J=10.3 Hz, 1H), 2.42 (s, 1H), 1.65 (s, 2H), 1.37-0.99 (m, 2H); MS (ESI) m/z: 414.2 [M+H⁺].

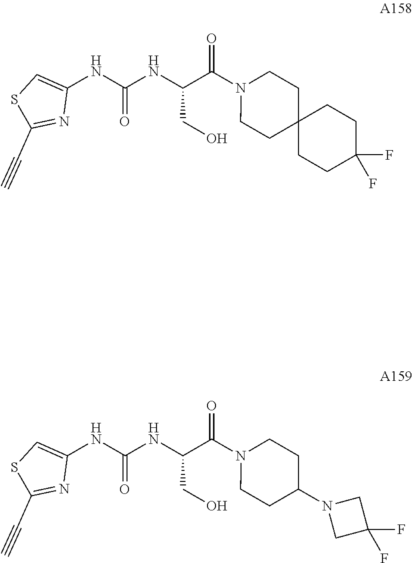 [SMILES: C#Cc1nc(NC(=O)N[C@@H](CO)C(=O)N2CCC3(CC2)CCC(F)(F)CC3)cs1 ; C#Cc1nc(NC(=O)N[C@@H](CO)C(=O)N2CCC(N3CC(F)(F)C3)CC2)cs1]

(R)-1-(1-(4-(3,3-Difluoroazetidin-1-yl)piperidin-1-yl)-3-hydroxy-1-oxopropan-2-yl)-3-(2-ethynylthiazol-4-yl)urea A160. ¹H NMR (400 MHz, DMSO-d₆) δ 9.56 (s, 1H), 7.28 (s, 1H), 6.76 (d, J=8.3 Hz, 1H), 4.98 (d, J=7.4 Hz, 1H), 4.89 (s, 1H), 4.77 (s, 1H), 3.99 (d, J=11.8 Hz, 1H), 3.85 (d, J=12.0 Hz, 1H), 3.56 (t, J=12.4 Hz, 4H), 3.50 (t, J=5.5 Hz, 2H), 3.21 (d, J=10.6 Hz, 1H), 2.96 (t, J=10.6 Hz, 1H), 2.42 (s, 1H), 1.65 (s, 2H), 1.46-0.93 (m, 2H); MS (ESI) m/z: 414.1 [M+H⁺].

(S)-1-(1-(4,4-Difluoropiperidin-1-yl)-3-hydroxy-1-oxopropan-2-yl)-3-(2-ethynyl-thiazol-4-yl)urea A161. ¹H NMR (400 MHz, DMSO-d₆) δ 9.58 (s, 1H), 7.28 (s, 1H), 6.80 (d, J=8.1 Hz, 1H), 5.07 (t, J=5.8 Hz, 1H), 4.89 (s, 1H), 4.82 (d, J=7.1 Hz, 1H), 3.55 (ddd, J=27.9, 23.8, 12.9 Hz, 6H), 2.11-1.87 (m, 4H); MS (ESI) m/z: 359.1 [M+H⁺].

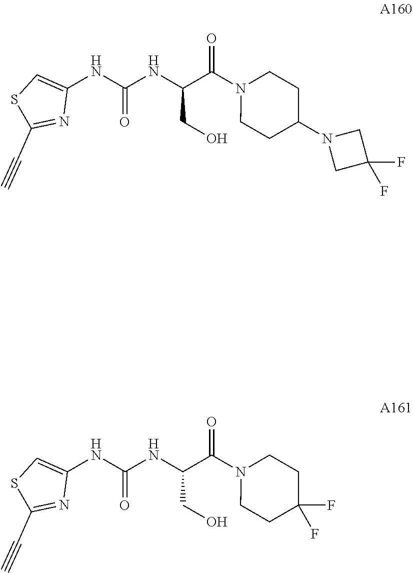 [SMILES: C#Cc1nc(NC(=O)N[C@H](CO)C(=O)N2CCC(N3CC(F)(F)C3)CC2)cs1 ; C#Cc1nc(NC(=O)N[C@@H](CO)C(=O)N2CCC(F)(F)CC2)cs1]

1-(6-Chloro-8-fluoro-7-(2-fluoro-6-methoxyphenyl)quinazolin-4-yl)-3-(2-ethynylthiazol-4-yl)urea A162. ¹H NMR (400 MHz, DMSO-d₆) δ 12.80 (s, 1H), 11.02 (s, 1H), 8.96 (s, 2H), 7.70 (s, 1H), 7.60 (td, J=8.4, 7.1 Hz, 1H), 7.11 (d, J=8.5 Hz, 1H), 7.04 (t, J=8.6 Hz, 1H), 5.00 (s, 1H), 3.79 (s, 3H); MS (ESI) m/z: 472.1 [M+H⁺].

1-(2-Ethynylthiazol-4-yl)-3-(7-(2-fluoro-6-hydroxyphenyl)quinazolin-4-yl)urea A163. ¹H NMR (400 MHz, DMSO-d₆) δ 9.84 (s, 1H), 9.22 (d, J=8.9 Hz, 1H), 8.84-8.72 (m, 2H), 8.49 (s, 1H), 7.79 (q, J=7.8 Hz, 1H), 7.30 (t, J=9.7 Hz, 2H), 4.78 (s, 1H); MS (ESI) m/z: 406.1 [M+H⁺].

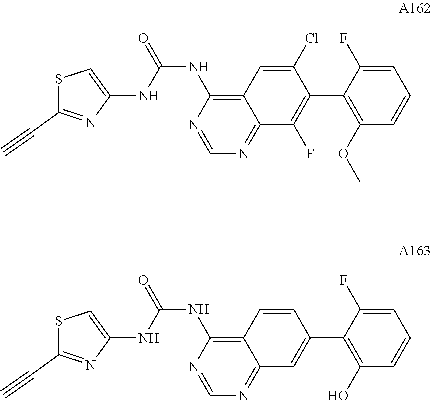 [SMILES: C#Cc1nc(NC(=O)Nc2ncnc3c(F)c(-c4c(F)cccc4OC)c(Cl)cc23)cs1 ; C#Cc1nc(NC(=O)Nc2ncnc3cc(-c4c(O)cccc4F)ccc23)cs1]

1-(2-Ethynylthiazol-4-yl)-3-(7-(2-fluoro-6-methoxyphenyl)quinazolin-4-yl)urea A164. ¹H NMR (400 MHz, DMSO-d₆) δ 9.47 (s, 1H), 8.83 (d, J=8.7 Hz, 1H), 8.37 (d, J=13.1 Hz, 2H), 8.13 (s, 1H), 7.54 (dd, J=15.1, 8.1 Hz, 1H), 7.05-6.89 (m, 2H), 4.44 (s, 1H), 3.94 (s, 3H); MS (ESI) m/z: 420.1 [M+H⁺].

1-(7-(Benzo[d]thiazol-7-yl)-6-chloro-8-fluoroquinazolin-4-yl)-3-(2-ethynyl-thiazol-4-yl)urea A165. ¹H NMR (400 MHz, DMSO-d₆) δ 12.75 (s, 1H), 11.14 (s, 1H), 9.47 (s, 1H), 8.99 (s, 2H), 8.28 (dd, J=8.2, 1.1 Hz, 1H), 7.77 (t, J=7.8 Hz, 1H), 7.71 (s, 1H), 7.66-7.58 (m, 1H), 4.99 (s, 1H); MS (ESI) m/z: 481.0 [M+H⁺].

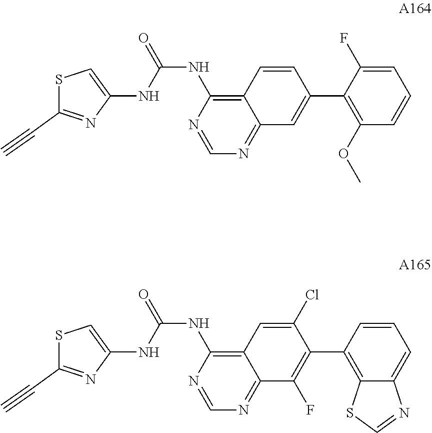 [SMILES: C#Cc1nc(NC(=O)Nc2ncnc3cc(-c4c(F)cccc4OC)ccc23)cs1 ; C#Cc1nc(NC(=O)Nc2ncnc3c(F)c(-c4cccc5ncsc45)c(Cl)cc23)cs1]

1-(7-(2-(1-Cyanocyclopropyl)pyridin-3-yl)quinazolin-4-yl)-3-(2-ethynylthiazol-4-yl)urea A166. ¹H NMR (400 MHz, DMSO-d₆) δ 13.00 (s, 1H), 10.92 (s, 1H), 8.96 (d, J=41.8 Hz, 2H), 8.64 (dd, J=4.8, 1.7 Hz, 1H), 8.06 (s, 1H), 7.93 (dd, J=7.8, 1.7 Hz, 1H), 7.89-7.78 (m, 1H), 7.70 (s, 1H), 7.57 (dd, J=7.7, 4.8 Hz, 1H), 4.99 (s, 1H), 1.82 (q, J=4.7 Hz, 2H), 1.59 (q, J=4.6 Hz, 2H); MS (ESI) m/z: 406.1 [M+H⁺].

1-((3′-(1-Cyanocyclopropyl)-[1,1′-biphenyl]-4-yl)methyl)-3-(2-ethynylthiazol-4-yl)urea A167. ¹H NMR (400 MHz, DMSO-d₆) δ 9.50 (s, 1H), 7.65 (d, J=8.2 Hz, 2H), 7.58 (d, J=8.1 Hz, 1H), 7.48 (dd, J=10.2, 4.8 Hz, 2H), 7.39 (d, J=8.2 Hz, 2H), 7.36 (d, J=7.8 Hz, 1H), 7.32 (s, 1H), 6.87 (s, 1H), 4.89 (s, 1H), 4.36 (d, J=5.9 Hz, 2H), 1.77 (q, J=4.7 Hz, 2H), 1.62 (q, J=5.1 Hz, 2H); MS (ESI) m/z: 399.1 [M+H⁺].

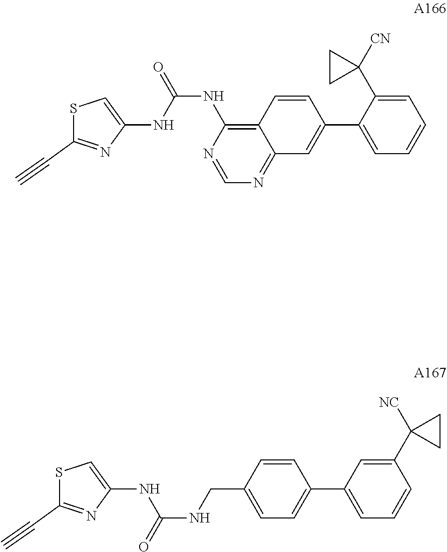 [SMILES: C#Cc1nc(NC(=O)Nc2ncnc3cc(-c4ccccc4C4(C#N)CC4)ccc23)cs1 ; C#Cc1nc(NC(=O)NCc2ccc(-c3cccc(C4(C#N)CC4)c3)cc2)cs1]

1-(2-Ethynylthiazol-4-yl)-3-((3′-(1-hydroxycyclopropyl)-[1,1′-biphenyl]-4-yl)methyl)urea A168. ¹H NMR (400 MHz, DMSO-d₆) δ 9.50 (s, 1H), 7.61 (d, J=8.2 Hz, 2H), 7.50 (d, J=1.6 Hz, 1H), 7.42 (d, J=7.9 Hz, 1H), 7.40-7.33 (m, 3H), 7.32 (s, 1H), 7.19 (d, J=7.7 Hz, 1H), 6.85 (t, J=5.9 Hz, 1H), 5.97 (s, 1H), 4.89 (s, 1H), 4.36 (d, J=5.9 Hz, 2H), 1.16-1.07 (m, 2H), 1.02 (dd, J=7.2, 4.9 Hz, 2H); MS (ESI) m/z: 390.1 [M+H⁺].

1-(2-Ethynylthiazol-4-yl)-3-((3′-propionyl-[1,1′-biphenyl]-4-yl)methyl)urea A169. ¹H NMR (400 MHz, DMSO-d₆) δ 9.52 (s, 1H), 8.17 (s, 1H), 7.92 (dd, J=13.9, 7.9 Hz, 2H), 7.71 (d, J=8.1 Hz, 2H), 7.68-7.53 (m, 1H), 7.50-7.38 (m, 2H), 7.32 (s, 1H), 6.88 (s, 1H), 4.89 (s, 1H), 4.37 (d, J=6.1 Hz, 2H), 3.13 (q, J=7.1 Hz, 2H), 1.11 (t, J=7.1 Hz, 3H); MS (ESI) m/z: 390.1 [M+H⁺].

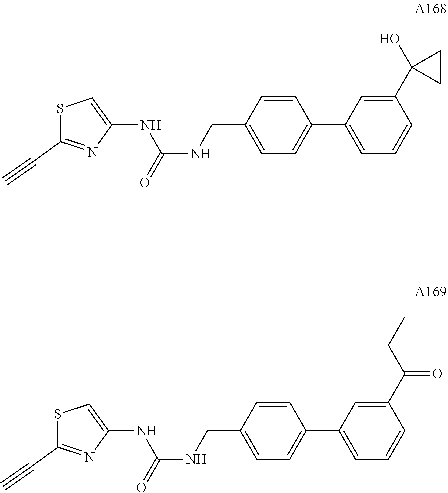 [SMILES: C#Cc1nc(NC(=O)NCc2ccc(-c3cccc(C4(O)CC4)c3)cc2)cs1 ; C#Cc1nc(NC(=O)NCc2ccc(-c3cccc(C(=O)CC)c3)cc2)cs1]

1-(2-Ethynylthiazol-4-yl)-3-((3′-(1-hydroxycyclobutyl)-[1,1′-biphenyl]-4-yl)-methyl)urea A170. ¹H NMR (400 MHz, DMSO-d₆) δ 9.49 (s, 1H), 7.71 (s, 1H), 7.63 (d, J=8.1 Hz, 2H), 7.48 (dd, J=9.3, 7.8 Hz, 2H), 7.43 (d, J=7.5 Hz, 1H), 7.39 (d, J=8.3 Hz, 2H), 7.32 (s, 1H), 6.85 (t, J=5.9 Hz, 1H), 5.52 (s, 1H), 4.89 (s, 1H), 4.36 (d, J=5.9 Hz, 2H), 2.47-2.41 (m, 2H), 2.29 (ddd, J=12.2, 9.5, 7.5 Hz, 2H), 2.02-1.85 (m, 1H), 1.76-1.59 (m, 1H); MS (ESI) m/z: 404.1 [M+H⁺].

1-(4-(2-(3,3-Difluoroazetidin-1-yl)-4-hydroxyquinazolin-5-yl)benzyl)-3-(2-ethynylthiazol-4-yl)urea A171. ¹H NMR (400 MHz, DMSO-d₆) δ 11.53 (s, 1H), 9.48 (s, 1H), 7.61-7.56 (m, 1H), 7.33 (dd, J=7.8, 1.6 Hz, 2H), 7.27-7.20 (m, 4H), 6.90 (dd, J=7.4, 1.0 Hz, 1H), 6.86 (t, J=6.0 Hz, 1H), 4.89 (s, 1H), 4.51 (t, J=12.5 Hz, 4H), 4.37 (d, J=5.9 Hz, 2H); MS (ESI) m/z: 493.2 [M+H⁺].

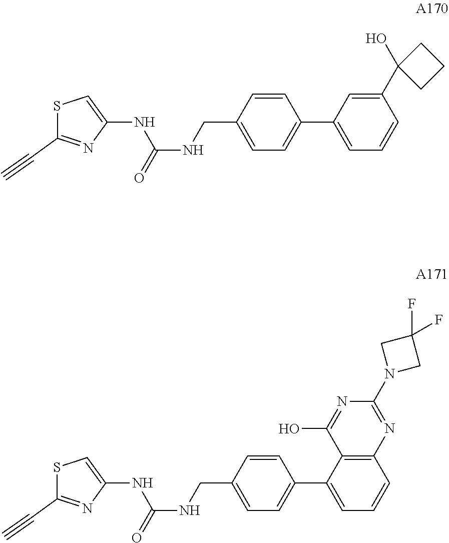 [SMILES: C#Cc1nc(NC(=O)NCc2ccc(-c3cccc(C4(O)CCC4)c3)cc2)cs1 ; C#Cc1nc(NC(=O)NCc2ccc(-c3cccc4nc(N5CC(F)(F)C5)nc(O)c34)cc2)cs1]

1-(2-Ethynylthiazol-4-yl)-3-(4-(4-hydroxy-2-(2-hydroxyethoxy)quinazolin-5-yl)-benzyl)urea A172. ¹H NMR (400 MHz, DMSO-d₆) δ 12.03 (s, 1H), 9.48 (s, 1H), 7.65 (t, J=7.8 Hz, 1H), 7.43 (d, J=8.0 Hz, 1H), 7.34 (s, 1H), 7.30-7.17 (m, 4H), 7.02 (d, J=7.2 Hz, 1H), 6.84 (t, J=5.8 Hz, 1H), 4.89 (s, 2H), 4.42 (t, J=4.9 Hz, 2H), 4.37 (d, J=5.7 Hz, 2H), 3.73 (t, J=4.6 Hz, 2H); MS (ESI) m/z: 462.1 [M+H⁺].

1-(2-Ethynylthiazol-4-yl)-3-(4-(4-(pyrrolidin-1-yl)pyridin-2-yl)benzyl)urea A173. ¹H NMR (400 MHz, DMSO-d₆) δ 9.68 (s, 1H), 8.16 (d, J=5.7 Hz, 1H), 7.99 (d, J=8.3 Hz, 2H), 7.36 (d, J=8.3 Hz, 2H), 7.32 (s, 1H), 7.16 (t, J=5.5 Hz, 1H), 6.90 (d, J=2.2 Hz, 1H), 6.42 (dd, J=5.8, 2.3 Hz, 1H), 4.90 (s, 1H), 4.36 (d, J=5.9 Hz, 2H), 3.33 (t, J=3.2 Hz, 5H), 2.00-1.93 (m, 5H).

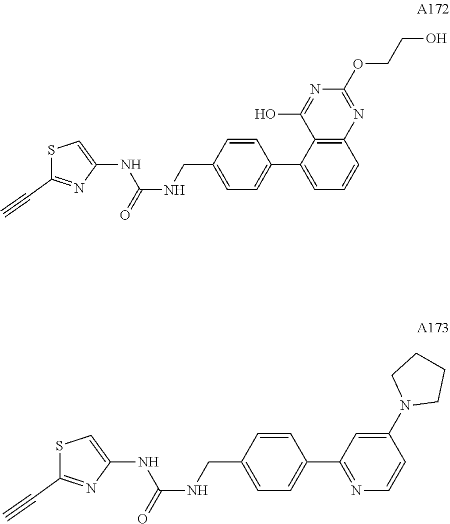 [SMILES: C#Cc1nc(NC(=O)NCc2ccc(-c3cccc4nc(OCCO)nc(O)c34)cc2)cs1 ; C#Cc1nc(NC(=O)NCc2ccc(-c3cc(N4CCCC4)ccn3)cc2)cs1]

1-(2-Ethynylthiazol-4-yl)-3-(4-(5-(pyrrolidin-1-yl)pyridin-3-yl)benzyl)urea A174. ¹H NMR (400 MHz, DMSO-d₆) δ 9.50 (s, 1H), 8.10 (d, J=1.8 Hz, 1H), 7.92 (d, J=2.7 Hz, 1H), 7.66 (d, J=8.2 Hz, 2H), 7.39 (d, J=8.2 Hz, 2H), 7.32 (s, 1H), 7.05-7.01 (m, 1H), 6.85 (t, J=5.9 Hz, 1H), 4.90 (s, 1H), 4.36 (d, J=5.9 Hz, 2H), 3.33 (d, J=6.6 Hz, 4H), 2.00-1.93 (m, 4H); MS (ESI) m/z: 404.1 [M+H⁺].

1-(2-Ethynylthiazol-4-yl)-3-(4-(2-(pyrrolidin-1-yl)pyridin-4-yl)benzyl)urea A175. ¹H NMR (400 MHz, DMSO-d₆) δ 9.51 (s, 1H), 8.09 (d, J=5.2 Hz, 1H), 7.70 (d, J=8.1 Hz, 2H), 7.40 (d, J=8.1 Hz, 2H), 7.32 (s, 1H), 6.85 (d, J=5.8 Hz, 1H), 6.80 (d, J=4.9 Hz, 1H), 6.61 (s, 1H), 4.90 (s, 1H), 4.36 (d, J=5.8 Hz, 2H), 3.44 (s, 4H), 1.95 (s, 4H); MS (ESI) m/z: 404.2 [M+H⁺].

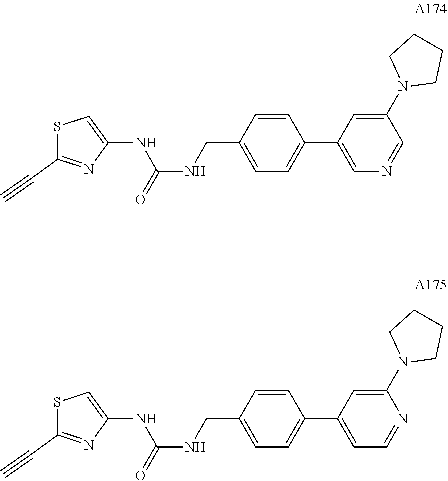 [SMILES: C#Cc1nc(NC(=O)NCc2ccc(-c3cncc(N4CCCC4)c3)cc2)cs1 ; C#Cc1nc(NC(=O)NCc2ccc(-c3ccnc(N4CCCC4)c3)cc2)cs1]

1-(2-Ethynylthiazol-4-yl)-3-(4-(6-(pyrrolidin-1-yl)pyridin-2-yl)benzyl)urea A176. ¹H NMR (400 MHz, DMSO-d₆) δ 9.50 (s, 1H), 8.01 (d, J=8.3 Hz, 2H), 7.54 (dd, J=8.2, 7.6 Hz, 1H), 7.35 (d, J=8.3 Hz, 2H), 7.32 (s, 1H), 7.09 (d, J=7.4 Hz, 1H), 6.85 (t, J=5.9 Hz, 1H), 6.39 (d, J=8.3 Hz, 1H), 4.89 (s, 1H), 4.35 (d, J=5.9 Hz, 2H), 3.46 (t, J=6.5 Hz, 4H), 2.04-1.89 (m, 4H); MS (ESI) m/z: 404.2 [M+H⁺].

1-(4-(Benzo[d]thiazol-7-yl)-2-cyanobenzyl)-3-(2-ethynylthiazol-4-yl)urea A177. ¹H NMR (400 MHz, DMSO-d₆) δ 13.37 (s, 1H), 9.80 (s, 1H), 9.49 (s, 1H), 8.54 (d, J=1.2 Hz, 1H), 8.17 (dd, J=8.1, 1.0 Hz, 1H), 8.00 (dd, J=7.9, 1.6 Hz, 1H), 7.83 (d, J=8.0 Hz, 1H), 7.77-7.70 (m, 1H), 7.65 (d, J=7.9 Hz, 2H), 5.00 (s, 2H), 4.97 (s, 1H); MS (ESI) m/z: 416.1 [M+H⁺].

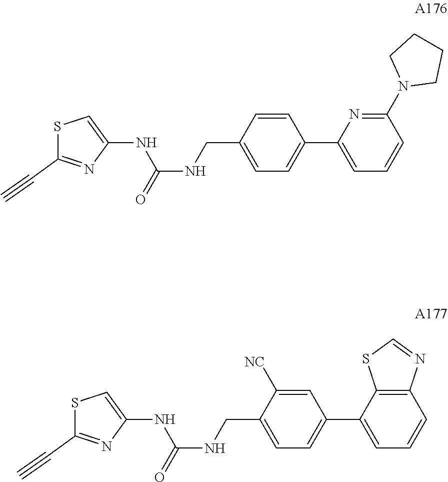 [SMILES: C#Cc1nc(NC(=O)NCc2ccc(-c3cccc(N4CCCC4)n3)cc2)cs1 ; C#Cc1nc(NC(=O)NCc2ccc(-c3cccc4ncsc34)cc2C#N)cs1]

1-(4-(Benzo[d]thiazol-7-yl)-2-cyanobenzyl)-3-(2-ethynylthiazol-4-yl)urea A178. ¹H NMR (400 MHz, DMSO-d₆) δ 9.68 (s, 1H), 8.84 (d, J=2.0 Hz, 1H), 8.09 (dd, J=8.1, 2.4 Hz, 1H), 7.65 (d, J=7.9 Hz, 1H), 7.53 (dd, J=8.8, 6.6 Hz, 2H), 7.44 (dd, J=10.6, 4.9 Hz, 2H), 7.31 (s, 1H), 7.04 (t, J=5.8 Hz, 1H), 4.90 (s, 1H), 4.47 (d, J=5.8 Hz, 2H), 1.78 (dd, J=7.7, 4.8 Hz, 2H), 1.65 (dd, J=7.9, 5.1 Hz, 2H).

1-((5-(Benzo[d]thiazol-7-yl)pyridin-2-yl)methyl)-3-(2-ethynylthiazol-4-yl)urea A179. ¹H NMR (400 MHz, DMSO-d₆) δ 9.69 (s, 1H), 9.48 (s, 1H), 8.88 (d, J=1.7 Hz, 1H), 8.20-8.09 (m, 2H), 7.71 (t, J=7.7 Hz, 1H), 7.65 (dd, J=7.4, 1.0 Hz, 1H), 7.53 (d, J=8.2 Hz, 1H), 7.33 (s, 1H), 7.09 (t, J=5.8 Hz, 1H), 4.90 (s, 1H), 4.53 (d, J=5.8 Hz, 2H); MS (ESI) m/z: 392.1 [M+H⁺].

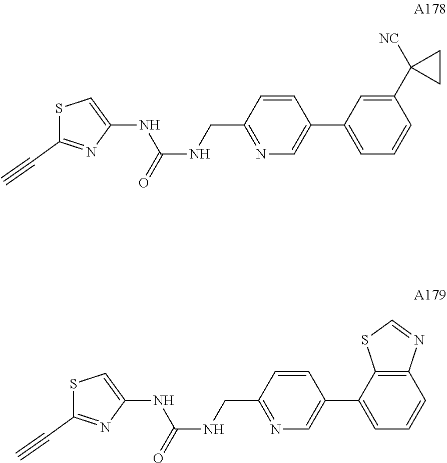 [SMILES: C#Cc1nc(NC(=O)NCc2ccc(-c3cccc(C4(C#N)CC4)c3)cn2)cs1 ; C#Cc1nc(NC(=O)NCc2ccc(-c3cccc4ncsc34)cn2)cs1]

1-((6-(3-(1-Cyanocyclopropyl)phenyl)pyridin-3-yl)methyl)-3-(2-ethynylthiazol-4-yl)urea A180. ¹H NMR (400 MHz, DMSO-d₆) δ 9.59 (s, 1H), 8.63 (d, J=1.6 Hz, 1H), 7.96-8.05 (m, 3H), 4.39 (d, J=9.2 Hz, 2H), 7.82 (dd, J=2.0, 4.0 Hz, 1H), 7.51 (t, J=7.6 Hz, 1H), 7.36-7.41 (m, 1H), 7.33 (s, 1H), 6.94 (t, J=5.6 Hz, 1H), 4.90 (s, 1H), 4.39 (d, J=6.0 Hz, 1H), 1.77-1.83 (m, 2H), 1.57-1.63 (m, 2H); MS (ESI) m/z: 401.1 [M+H⁺].

(R)-1-(1-(3′-(3,3-Difluoropyrrolidin-1-yl)-[1,1′-biphenyl]-4-yl)-2-hydroxyethyl)-3-(2-ethynylthiazol-4-yl)urea A181. ¹H NMR (400 MHz, DMSO-d₆) δ 9.50 (s, 1H), 7.61 (d, J=8.3 Hz, 2H), 7.37 (d, J=8.2 Hz, 2H), 7.29 (d, J=7.9 Hz, 1H), 7.26 (s, 1H), 7.06 (d, J=7.7 Hz, 1H), 6.96 (d, J=7.8 Hz, 1H), 6.80 (s, 1H), 6.61 (dd, J=8.1, 2.0 Hz, 1H), 5.04 (t, J=5.2 Hz, 1H), 4.89 (s, 1H), 4.78 (d, J=7.6 Hz, 1H), 3.76 (t, J=13.5 Hz, 2H), 3.68 (d, J=5.0 Hz, 1H), 3.61 (d, J=5.5 Hz, 1H), 3.53 (t, J=7.2 Hz, 2H), 2.57 (dd, J=14.6, 7.2 Hz, 2H); MS (ESI) m/z: 469.2 [M+H⁺].

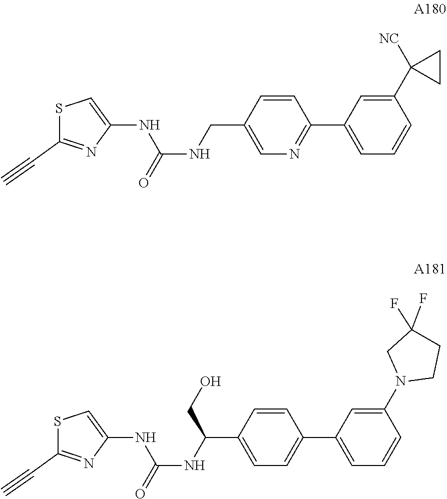 [SMILES: C#Cc1nc(NC(=O)NCc2ccc(-c3cccc(C4(C#N)CC4)c3)nc2)cs1 ; C#Cc1nc(NC(=O)N[C@@H](CO)c2ccc(-c3cccc(N4CCC(F)(F)C4)c3)cc2)cs1]

(R)-1-(1-(4-Cyclohexylphenyl)-2-hydroxyethyl)-3-(2-ethynylthiazol-4-yl)urea A182. ¹H NMR (400 MHz, DMSO-d₆) δ 9.43 (s, 1H), 7.24 (s, 1H), 7.18 (q, J=8.3 Hz, 4H), 6.94 (d, J=7.9 Hz, 1H), 4.97 (t, J=5.1 Hz, 1H), 4.89 (s, 1H), 4.70 (dd, J=12.9, 5.4 Hz, 1H), 3.69-3.43 (m, 2H), 1.85-1.64 (m, 5H), 1.49-1.13 (m, 5H); MS (ESI) m/z: 370.2 [M+H⁺].

(R)-1-(2-Ethynylthiazol-4-yl)-3-(2-hydroxy-1-(2′,3′,4′,5′-tetrahydro-[1,1′-biphenyl]-4-yl)ethyl)urea A183. ¹H NMR (400 MHz, DMSO-d₆) δ 9.45 (s, 1H), 7.34 (d, J=8.3 Hz, 2H), 7.23 (d, J=9.0 Hz, 3H), 6.97 (d, J=7.8 Hz, 1H), 6.10 (dd, J=4.7, 3.1 Hz, 1H), 4.98 (t, J=5.2 Hz, 1H), 4.89 (s, 1H), 4.72 (dd, J=12.9, 5.3 Hz, 1H), 3.63 (dd, J=10.4, 5.3 Hz, 1H), 3.56 (dd, J=10.9, 5.5 Hz, 1H), 2.33 (dd, J=4.0, 2.1 Hz, 2H), 2.16 (dd, J=6.1, 2.4 Hz, 2H), 1.75-1.68 (m, 2H), 1.64-1.54 (m, 2H); MS (ESI) m/z: 368.2 [M+H⁺].

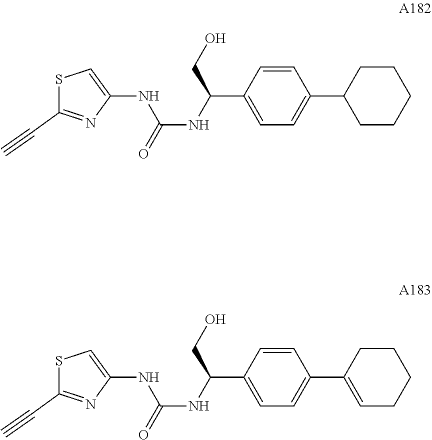 [SMILES: C#Cc1nc(NC(=O)N[C@@H](CO)c2ccc(C3CCCCC3)cc2)cs1 ; C#Cc1nc(NC(=O)N[C@@H](CO)c2ccc(C3=CCCCC3)cc2)cs1]

(R)-1-(2-Ethynylthiazol-4-yl)-3-(2-hydroxy-1-(4-(1-methylpiperidin-4-yl)phenyl)-ethyl)urea A184. ¹H NMR (400 MHz, DMSO-d₆) δ 9.46 (d, J=8.6 Hz, 1H), 8.21 (d, J=13.5 Hz, 1H), 7.32-7.13 (m, 5H), 6.98 (d, J=8.1 Hz, 1H), 4.89 (s, 1H), 4.70 (dd, J=12.8, 5.4 Hz, 1H), 3.59 (ddd, J=16.7, 10.8, 5.4 Hz, 3H), 2.91 (d, J=10.2 Hz, 2H), 2.31-2.03 (m, 5H), 1.78-1.57 (m, 4H); MS (ESI) m/z: 385.5 [M+H⁺].

(R)-1-(2-Ethynylthiazol-4-yl)-3-(2-hydroxy-1-(4-(1-methyl-1,2,3,6-tetrahydro-pyridin-4-yl)phenyl)ethyl)urea A185. ¹H NMR (400 MHz, DMSO-d₆) δ 9.75 (s, 1H), 7.48 (s, 1H), 7.36 (d, J=8.3 Hz, 2H), 7.28-7.22 (m, 3H), 6.10 (t, J=3.4 Hz, 1H), 4.88 (s, 1H), 4.71 (dd, J=12.6, 5.8 Hz, 1H), 3.62 (dd, J=10.9, 4.7 Hz, 1H), 3.55 (dd, J=10.9, 6.1 Hz, 1H), 3.00 (d, J=2.9 Hz, 2H), 2.56 (t, J=5.6 Hz, 2H), 2.52 (d, J=1.9 Hz, 2H), 2.27 (s, 3H); MS (ESI) m/z: 383.5 [M+H⁺].

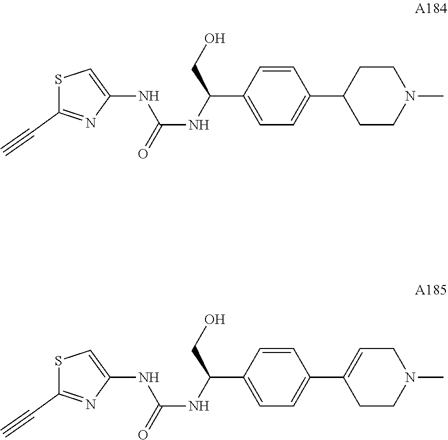

(R)-1-(2-Ethynylthiazol-4-yl)-3-(2-hydroxy-1-(3′-(methylsulfonyl)-[1,1′-biphenyl]-4-yl)ethyl)urea A186. ¹H NMR (400 MHz, DMSO-d₆) δ 9.50 (s, 1H), 8.14 (t, J=1.7 Hz, 1H), 8.05-7.96 (m, 1H), 7.90 (ddd, J=7.8, 1.7, 1.1 Hz, 1H), 7.77-7.69 (m, 3H), 7.45 (d, J=8.3 Hz, 2H), 7.26 (s, 1H), 7.07 (d, J=7.8 Hz, 1H), 5.06 (t, J=5.2 Hz, 1H), 4.90 (s, 1H), 4.82 (dd, J=12.8, 5.2 Hz, 1H), 3.67 (dtd, J=21.8, 10.8, 5.2 Hz, 2H), 3.29 (s, 3H); MS (ESI) m/z: 448.1 [M+H⁺].

(R)-1-(1-(3′-(1-Cyanocyclopropyl)-[1,1′-biphenyl]-4-yl)-2-hydroxyethyl)-3-(2-ethynylthiazol-4-yl)urea A187. ¹H NMR (400 MHz, DMSO-d₆) δ 9.50 (s, 1H), 7.63 (d, J=8.3 Hz, 2H), 7.57 (d, J=8.0 Hz, 1H), 7.48 (dd, J=8.8, 6.6 Hz, 2H), 7.40 (d, J=8.2 Hz, 2H), 7.38-7.32 (m, 1H), 7.26 (s, 1H), 7.06 (d, J=7.8 Hz, 1H), 5.05 (t, J=5.1 Hz, 1H), 4.89 (s, 1H), 4.80 (d, J=7.5 Hz, 1H), 3.69 (dt, J=10.1, 4.9 Hz, 1H), 3.61 (dt, J=10.8, 5.5 Hz, 1H), 1.77 (q, J=4.7 Hz, 2H), 1.62 (q, J=5.2 Hz, 2H); MS (ESI) m/z: 429.1 [M+H⁺].

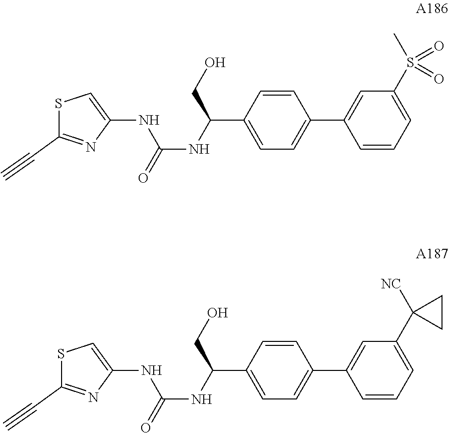 [SMILES: C#Cc1nc(NC(=O)N[C@@H](CO)c2ccc(-c3cccc(S(C)(=O)=O)c3)cc2)cs1 ; C#Cc1nc(NC(=O)N[C@@H](CO)c2ccc(-c3cccc(C4(C#N)CC4)c3)cc2)cs1]

1-((1R)-1-(3′-(2,2-Difluorocyclopropyl)-[1,1′-biphenyl]-4-yl)-2-hydroxyethyl)-3-(2-ethynylthiazol-4-yl)urea A188. ¹H NMR (400 MHz, DMSO-d₆) δ 9.51 (s, 1H), 7.62 (d, J=8.2 Hz, 2H), 7.54 (dd, J=6.7, 5.6 Hz, 2H), 7.41 (dd, J=14.9, 7.9 Hz, 3H), 7.25 (d, J=6.3 Hz, 2H), 7.08 (d, J=7.8 Hz, 1H), 5.06 (t, J=4.9 Hz, 1H), 4.89 (s, 1H), 4.80 (dd, J=12.7, 5.3 Hz, 1H), 3.69 (dt, J=9.5, 4.6 Hz, 1H), 3.61 (dt, J=10.6, 5.2 Hz, 1H), 3.07 (td, J=12.4, 8.4 Hz, 1H), 2.14-1.90 (m, 2H).

(R)-1-(2-Ethynylthiazol-4-yl)-3-(2-hydroxy-1-(3′-(1-hydroxycyclobutyl)-[1,1′-biphenyl]-4-yl)ethyl)urea A189. ¹H NMR (400 MHz, DMSO-d₆) δ 9.49 (s, 1H), 7.69 (s, 1H), 7.61 (d, J=8.2 Hz, 2H), 7.52-7.45 (m, 2H), 7.45-7.35 (m, 3H), 7.26 (s, 1H), 7.04 (d, J=7.8 Hz, 1H), 5.52 (s, 1H), 5.04 (t, J=5.2 Hz, 1H), 4.89 (s, 1H), 4.80 (dd, J=12.7, 5.3 Hz, 1H), 3.66 (dtd, J=22.0, 10.8, 5.2 Hz, 2H), 2.46-2.39 (m, 2H), 2.29 (ddd, J=12.2, 9.4, 7.5 Hz, 2H), 1.94 (ddd, J=14.8, 9.5, 5.1 Hz, 1H), 1.77-1.59 (m, 1H); MS (ESI) m/z: 434.1 [M+H⁺].

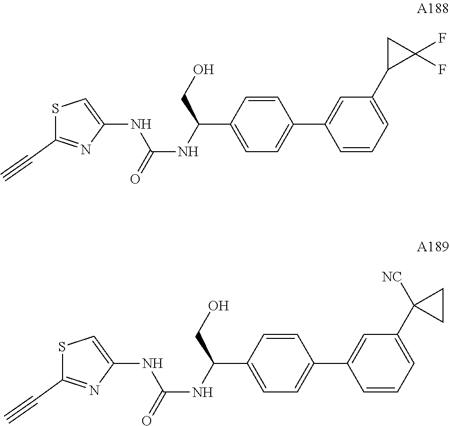 [SMILES: C#Cc1nc(NC(=O)N[C@@H](CO)c2ccc(-c3cccc(C4CC4(F)F)c3)cc2)cs1 ; C#Cc1nc(NC(=O)N[C@@H](CO)c2ccc(-c3cccc(C4(C#N)CC4)c3)cc2)cs1]

(R)-1-(1-(3′-(Azetidin-1-yl)-[1,1′-biphenyl]-4-yl)-2-hydroxyethyl)-3-(2-ethynyl-thiazol-4-yl)urea A190. ¹H NMR (400 MHz, DMSO-d₆) δ 9.49 (s, 1H), 7.54 (dd, J=15.8, 8.4 Hz, 2H), 7.37 (t, J=6.8 Hz, 2H), 7.25 (s, 1H), 7.23 (t, J=7.8 Hz, 1H), 7.05 (d, J=7.8 Hz, 1H), 6.91 (d, J=8.1 Hz, 1H), 6.59 (t, J=1.9 Hz, 1H), 6.39 (dd, J=8.0, 1.5 Hz, 1H), 5.04 (t, J=5.1 Hz, 1H), 4.89 (s, 1H), 4.78 (dd, J=12.8, 5.2 Hz, 1H), 3.84 (t, J=7.2 Hz, 3H), 3.74-3.63 (m, 1H), 3.60 (dt, J=10.9, 5.5 Hz, 1H), 2.55-2.51 (m, 1H), 2.38-2.18 (m, 2H); MS (ESI) m/z: 419.2 [M+H⁺].

(R)-1-(1-(3′-(3,3-Difluoroazetidin-1-yl)-[1,1′-biphenyl]-4-yl)-2-hydroxyethyl)-3-(2-ethynylthiazol-4-yl)urea A191. ¹H NMR (400 MHz, DMSO-d₆) δ 9.49 (s, 1H), 7.60 (d, J=8.3 Hz, 2H), 7.37 (d, J=8.2 Hz, 2H), 7.30 (t, J=7.8 Hz, 1H), 7.26 (s, 1H), 7.05 (t, J=7.8 Hz, 2H), 6.79-6.75 (m, 1H), 6.56 (dd, J=8.0, 1.7 Hz, 1H), 5.04 (t, J=5.2 Hz, 1H), 4.89 (s, 1H), 4.79 (dd, J=12.7, 5.2 Hz, 1H), 4.31 (t, J=12.3 Hz, 4H), 3.65 (dtd, J=21.9, 10.8, 5.2 Hz, 2H).

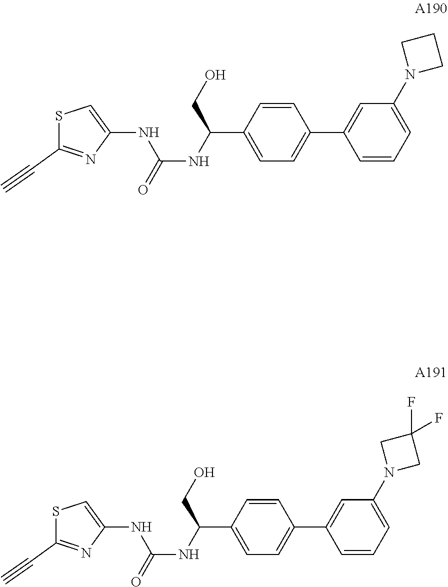 [SMILES: C#Cc1nc(NC(=O)N[C@@H](CO)c2ccc(-c3cccc(N4CCC4)c3)cc2)cs1 ; C#Cc1nc(NC(=O)N[C@@H](CO)c2ccc(-c3cccc(N4CC(F)(F)C4)c3)cc2)cs1]

1-(2-Ethynylthiazol-4-yl)-3-((1R)-2-hydroxy-1-(4-(1-methylpiperidin-3-yl)-phenyl)ethyl)urea A192. ¹H NMR (400 MHz, DMSO-d₆) δ 9.47 (s, 1H), 7.21 (dd, J=12.7, 9.5 Hz, 5H), 7.00 (d, J=7.3 Hz, 1H), 4.89 (s, 1H), 4.71 (dd, J=12.9, 5.4 Hz, 1H), 3.63 (dd, J=10.8, 4.8 Hz, 1H), 3.54 (dd, J=10.8, 5.9 Hz, 1H), 2.85 (d, J=8.9 Hz, 2H), 2.73 (t, J=11.4 Hz, 1H), 2.23 (s, 3H), 2.10-1.91 (m, 2H), 1.75 (dd, J=25.1, 12.7 Hz, 2H), 1.62 (t, J=12.5 Hz, 1H), 1.38 (qd, J=12.5, 2.8 Hz, 1H).

(R)-1-(2-Ethynylthiazol-4-yl)-3-(2-hydroxy-1-(4-(1-methyl-1,2,5,6-tetrahydro-pyridin-3-yl)phenyl)ethyl)urea A193. ¹H NMR (400 MHz, DMSO-d₆) δ 9.49 (s, 1H), 8.22 (s, 1H), 7.34 (d, J=8.3 Hz, 2H), 7.25 (d, J=7.0 Hz, 3H), 7.04 (d, J=7.8 Hz, 1H), 6.17-6.08 (m, 1H), 4.89 (s, 1H), 4.73 (dd, J=12.9, 5.3 Hz, 1H), 3.64 (dd, J=10.8, 4.8 Hz, 1H), 3.56 (dd, J=10.8, 5.9 Hz, 1H), 3.20 (d, J=1.8 Hz, 2H), 2.35 (s, 3H), 2.27 (d, J=3.5 Hz, 2H).

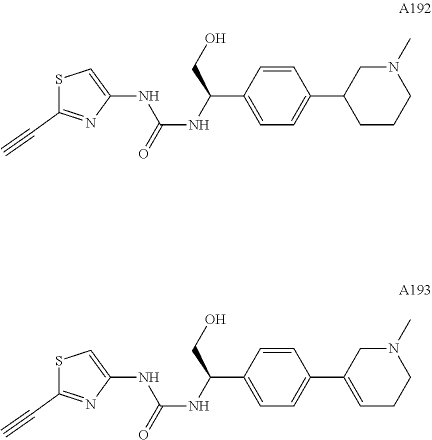

1-((1R)-1-(4-(1-Acetylpiperidin-3-yl)phenyl)-2-hydroxyethyl)-3-(2-ethynyl-thiazol-4-yl)urea A194. ¹H NMR (400 MHz, DMSO-d₆) δ 9.43 (s, 1H), 7.36-7.08 (m, 5H), 6.96 (d, J=7.8 Hz, 1H), 4.98 (dd, J=7.7, 5.0 Hz, 1H), 4.87 (s, 1H), 4.71 (dd, J=12.5, 5.4 Hz, 1H), 4.40 (t, J=12.8 Hz, 1H), 3.78 (dd, J=26.6, 13.3 Hz, 1H), 3.68-3.48 (m, 2H), 3.04 (td, J=13.0, 2.2 Hz, 1H), 2.71-2.59 (m, 1H), 1.99 (d, J=8.4 Hz, 3H), 1.87 (d, J=11.0 Hz, 1H), 1.79-1.58 (m, 2H), 1.55-1.23 (m, 1H); MS (ESI) m/z: 413.1 [M+H⁺].

(R)-1-(2-Ethynylthiazol-4-yl)-3-(2-hydroxy-1-(4-(piperidin-1-yl)phenyl)ethyl)-urea A195. ¹H NMR (400 MHz, DMSO-d₆) δ 9.39 (s, 1H), 7.25 (s, 1H), 7.12 (d, J=8.7 Hz, 2H), 6.87 (d, J=8.7 Hz, 2H), 6.84 (d, J=8.1 Hz, 1H), 4.91 (t, J=5.3 Hz, 1H), 4.89 (s, 1H), 4.64 (d, J=7.5 Hz, 1H), 3.59 (dt, J=10.2, 5.0 Hz, 1H), 3.56-3.47 (m, 1H), 3.17-2.97 (m, 4H), 1.66-1.57 (m, 4H), 1.54 (dd, J=15.9, 10.0 Hz, 2H); MS (ESI) m/z: 413.1 [M+H⁺].

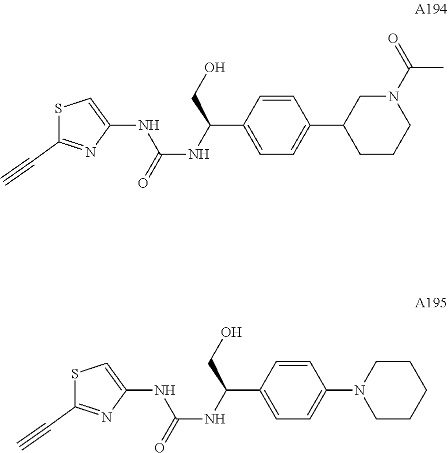

(S)-1-(1-(4-(Benzo[d]thiazol-7-yl)phenyl)-2-cyanoethyl)-3-(2-ethynylthiazol-4-yl)urea A196. ¹H NMR (400 MHz, DMSO-d₆) δ 9.55 (s, 1H), 9.46 (s, 1H), 8.12 (dd, J=8.0, 1.0 Hz, 1H), 7.78 (d, J=8.3 Hz, 2H), 7.68 (t, J=7.7 Hz, 1H), 7.65-7.56 (m, 3H), 7.34 (s, 1H), 7.28 (d, J=8.2 Hz, 1H), 5.23 (dd, J=14.6, 6.6 Hz, 1H), 4.91 (s, 1H), 3.21 (d, J=6.6 Hz, 2H); MS (ESI) m/z: 430.1 [M+H⁺].

(R)-1-(1-(4-(Benzo[d]thiazol-7-yl)phenyl)-2-(methylsulfonyl)ethyl)-3-(2-ethynyl-thiazol-4-yl)urea A197. ¹H NMR (400 MHz, DMSO-d₆) δ 9.66 (s, 1H), 9.46 (s, 1H), 8.11 (dd, J=8.0, 1.0 Hz, 1H), 7.75 (d, J=8.3 Hz, 2H), 7.67 (t, J=7.7 Hz, 1H), 7.59 (d, J=8.1 Hz, 3H), 7.32 (s, 1H), 7.20 (d, J=8.0 Hz, 1H), 5.42 (td, J=8.9, 4.7 Hz, 1H), 4.90 (s, 1H), 3.85 (dd, J=14.6, 9.3 Hz, 1H), 3.71 (dd, J=14.7, 4.6 Hz, 1H), 2.98 (s, 3H); MS (ESI) m/z: 483.1 [M+H⁺].

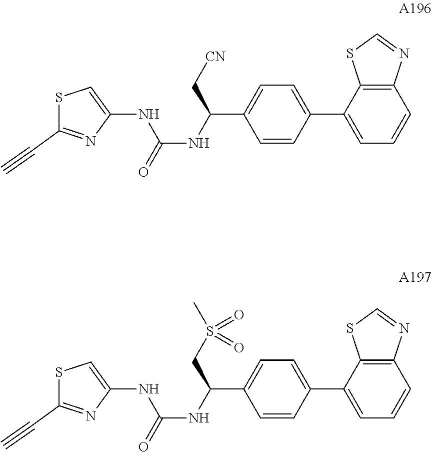 [SMILES: C#Cc1nc(NC(=O)N[C@@H](CC#N)c2ccc(-c3cccc4ncsc34)cc2)cs1 ; C#Cc1nc(NC(=O)N[C@@H](CS(C)(=O)=O)c2ccc(-c3cccc4ncsc34)cc2)cs1]

(R)-2-(4-(Benzo[d]thiazol-7-yl)phenyl)-2-(3-(2-ethynylthiazol-4-yl)ureido)-ethane-1-sulfonamide A198. ¹H NMR (400 MHz, DMSO-d₆) δ 9.61 (s, 1H), 9.46 (s, 1H), 8.11 (dd, J=8.0, 0.9 Hz, 1H), 7.73 (d, J=8.3 Hz, 2H), 7.67 (t, J=7.8 Hz, 1H), 7.59 (d, J=7.0 Hz, 1H), 7.56 (d, J=8.3 Hz, 2H), 7.30 (s, 1H), 7.13 (d, J=5.9 Hz, 1H), 6.98 (s, 2H), 5.34 (dt, J=12.5, 6.3 Hz, 1H), 4.90 (s, 1H), 3.64 (dd, J=14.4, 8.7 Hz, 1H), 3.51 (dd, J=14.4, 4.6 Hz, 1H); MS (ESI) m/z: 484.1 [M+H⁺].

(R)-2-(4-(Benzo[d]thiazol-7-yl)phenyl)-2-(3-(2-ethynylthiazol-4-yl)ureido)-N-methylethane-1-sulfonamide A199. ¹H NMR (400 MHz, DMSO-d₆) δ 9.61 (s, 1H), 9.46 (s, 1H), 8.11 (dd, J=8.1, 1.0 Hz, 1H), 7.74 (d, J=8.3 Hz, 2H), 7.70-7.65 (m, 1H), 7.59 (dd, J=7.3, 5.4 Hz, 3H), 7.31 (s, 1H), 7.12 (d, J=7.8 Hz, 1H), 6.99 (q, J=4.8 Hz, 1H), 5.28 (dd, J=13.0, 8.0 Hz, 1H), 4.90 (s, 1H), 3.66 (dd, J=14.5, 8.5 Hz, 1H), 3.55 (dd, J=14.5, 5.0 Hz, 1H), 2.62 (d, J=4.9 Hz, 3H); MS (ESI) nm/z: 498.1 [M+H⁺].

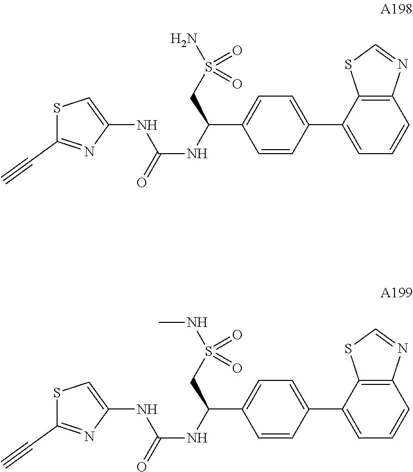 [SMILES: C#Cc1nc(NC(=O)N[C@@H](CS(N)(=O)=O)c2ccc(-c3cccc4ncsc34)cc2)cs1 ; C#Cc1nc(NC(=O)N[C@@H](CS(=O)(=O)NC)c2ccc(-c3cccc4ncsc34)cc2)cs1]

(R)-1-(1-(4-(Benzo[d]thiazol-7-yl)phenyl)-2-methoxyethyl)-3-(2-ethynylthiazol-4-yl)urea A200. ¹H NMR (400 MHz, DMSO-d₆) δ 9.48 (s, 1H), 9.45 (s, 1H), 8.10 (dd, J=8.1, 1.1 Hz, 1H), 7.71 (d, J=8.3 Hz, 2H), 7.69-7.63 (m, 1H), 7.61-7.56 (m, 1H), 7.51 (d, J=8.2 Hz, 2H), 7.28 (s, 1H), 7.15 (d, J=7.9 Hz, 1H), 5.09-4.96 (m, 1H), 4.90 (s, 1H), 3.65 (d, J=5.3 Hz, 2H), 3.31 (s, 3H); MS (ESI) m/z: 435.0 [M+H⁺].

(R)-2-(4-(Benzo[d]thiazol-7-yl)phenyl)-2-(3-(2-ethynylthiazol-4-yl)ureido)ethyl acetate A201. ¹H NMR (400 MHz, DMSO-d₆) δ 9.49 (s, 1H), 9.46 (s, 1H), 8.11 (dd, J=8.0, 1.1 Hz, 1H), 7.75 (d, J=8.3 Hz, 2H), 7.70-7.64 (m, 1H), 7.60 (d, J=6.7 Hz, 1H), 7.55 (d, J=8.2 Hz, 2H), 7.30 (s, 1H), 7.17 (d, J=8.3 Hz, 1H), 5.15 (dd, J=13.7, 5.8 Hz, 1H), 4.91 (s, 1H), 4.38-4.29 (m, 2H), 2.04 (s, 3H); MS (ESI) m/z: 463.0 [M+H⁺].

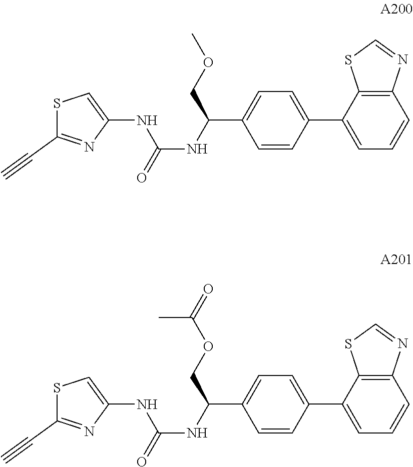 [SMILES: C#Cc1nc(NC(=O)N[C@@H](COC)c2ccc(-c3cccc4ncsc34)cc2)cs1 ; C#Cc1nc(NC(=O)N[C@@H](COC(C)=O)c2ccc(-c3cccc4ncsc34)cc2)cs1]

(R)-2-(4-(Benzo[d]thiazol-7-yl)phenyl)-2-(3-(2-ethynylthiazol-4-yl)ureido)ethyl pivalate A202. ¹H NMR (400 MHz, DMSO-d₆) δ 9.59 (s, 1H), 9.45 (s, 1H), 8.11 (d, J=8.1 Hz, 1H), 7.73 (d, J=8.2 Hz, 2H), 7.67 (t, J=7.8 Hz, 1H), 7.56 (dd, J=14.3, 7.8 Hz, 3H), 7.29 (s, 1H), 7.12 (d, J=8.5 Hz, 1H), 5.21 (dd, J=13.8, 5.8 Hz, 1H), 4.91 (s, 1H), 4.39 (dd, J=11.2, 6.5 Hz, 1H), 4.29 (dd, J=11.1, 4.9 Hz, 1H), 1.09 (s, 9H); MS (ESI) m/z: 505.1 [M+H⁺].

(R)-1-(1-(4-(Benzo[d]thiazol-7-yl)phenyl)-2-hydroxy-2-methylpropyl)-3-(2-ethynylthiazol-4-yl)urea A203. ¹H NMR (400 MHz, DMSO-d₆) δ 9.55 (s, 1H), 9.45 (s, 1H), 8.09 (d, J=7.4 Hz, 1H), 7.66 (dd, J=7.9, 5.7 Hz, 3H), 7.59 (d, J=7.1 Hz, 1H), 7.49 (d, J=8.2 Hz, 2H), 7.30-7.21 (m, 2H), 4.89 (s, 1H), 4.80 (s, 1H), 4.62 (d, J=8.8 Hz, 1H), 1.24 (s, 3H), 1.06 (s, 3H); MS (ESI) m/z: 449.1 [M+H⁺].

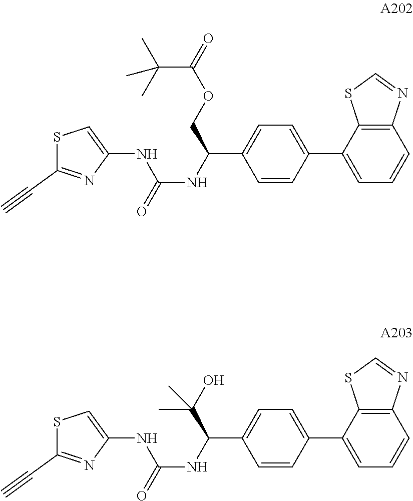 [SMILES: C#Cc1nc(NC(=O)N[C@@H](COC(=O)C(C)(C)C)c2ccc(-c3cccc4ncsc34)cc2)cs1 ; C#Cc1nc(NC(=O)N[C@H](c2ccc(-c3cccc4ncsc34)cc2)C(C)(C)O)cs1]

1-((1R)-1-(4-(Benzo[d]thiazol-7-yl)phenyl)-2-hydroxypropyl)-3-(2-ethynyl-thiazol-4-yl)urea A204. ¹H NMR (400 MHz, DMSO-d₆) δ 9.62 (s, 1H), 9.45 (s, 1H), 8.10 (d, J=7.8 Hz, 1H), 7.67 (dd, J=14.3, 8.0 Hz, 3H), 7.58 (d, J=7.3 Hz, 1H), 7.48 (d, J=8.1 Hz, 2H), 7.26 (s, 1H), 7.14 (d, J=8.5 Hz, 1H), 5.76 (s, 1H), 5.02 (t, J=4.8 Hz, 1H), 4.90 (s, 1H), 4.71 (dd, J=8.4, 2.5 Hz, 1H), 3.99 (dd, J=8.3, 5.1 Hz, 1H), 1.18 (d, J=6.2 Hz, 3H); MS (ESI) m/z: 436.1 [M+H⁺].

1-(4-(Benzo[d]thiazol-7-yl)benzyl)-1-(2-cyanoethyl)-3-(2-ethynylthiazol-4-yl)urea A205. ¹H NMR (400 MHz, DMSO-d₆) δ 10.04 (s, 1H), 9.45 (s, 1H), 8.15-8.05 (m, 1H), 7.72 (d, J=8.1 Hz, 2H), 7.67 (t, J=7.8 Hz, 1H), 7.58 (d, J=7.3 Hz, 1H), 7.52 (s, 1H), 7.43 (d, J=8.2 Hz, 2H), 4.90 (s, 1H), 4.78 (s, 2H), 3.70 (t, J=6.8 Hz, 2H), 2.81 (t, J=6.8 Hz, 2H); MS (ESI) m/z: 444.1 [M+H⁺].

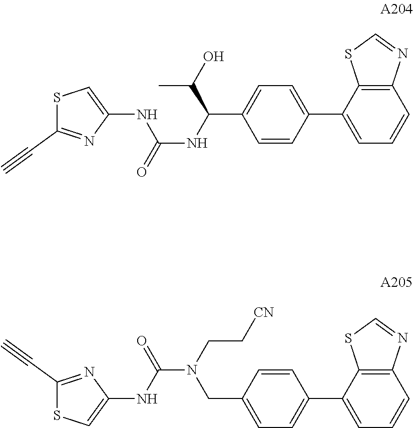 [SMILES: C#Cc1nc(NC(=O)N[C@H](c2ccc(-c3cccc4ncsc34)cc2)C(C)O)cs1 ; C#Cc1nc(NC(=O)N(CCC#N)Cc2ccc(-c3cccc4ncsc34)cc2)cs1]

1-(4-(Benzo[d]thiazol-7-yl)benzyl)-3-(2-ethynylthiazol-4-yl)-1-(2-hydroxyethyl)-urea A206. ¹H NMR (400 MHz, DMSO-d₆) δ 9.87 (s, 1H), 9.45 (s, 1H), 8.10 (dd, J=8.0, 0.9 Hz, 1H), 7.74-7.69 (m, 2H), 7.67 (t, J=7.8 Hz, 1H), 7.58 (d, J=7.2 Hz, 1H), 7.48-7.42 (m, 3H), 5.44 (s, 1H), 4.90 (s, 1H), 4.69 (s, 2H), 3.62 (dt, J=9.6, 4.9 Hz, 2H), 3.46 (t, J=5.0 Hz, 2H); MS (ESI) m/z: 435.1 [M+H⁺].

1-(4-(Benzo[d]thiazol-7-yl)benzyl)-3-(2-ethynylthiazol-4-yl)-1-(2-(methyl-sulfonyl)ethyl)urea A207. ¹H NMR (400 MHz, DMSO-d₆) δ 9.98 (s, 1H), 9.45 (s, 1H), 8.11 (dd, J=8.0, 1.1 Hz, 1H), 7.74 (d, J=8.3 Hz, 2H), 7.69-7.64 (m, 1H), 7.59 (d, J=6.7 Hz, 1H), 7.52 (s, 1H), 7.43 (d, J=8.2 Hz, 2H), 4.90 (s, 1H), 4.79 (s, 2H), 3.83-3.72 (m, 2H), 3.48 (t, J=7.1 Hz, 2H), 3.04 (s, 3H).

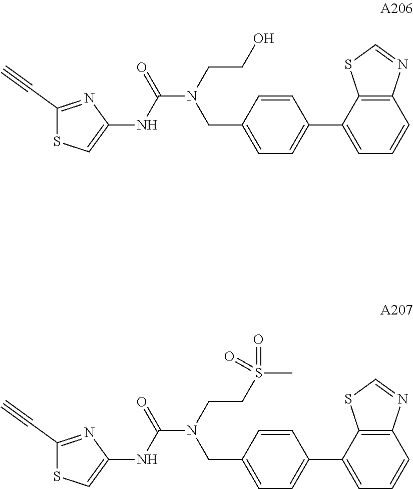 [SMILES: C#Cc1nc(NC(=O)N(CCO)Cc2ccc(-c3cccc4ncsc34)cc2)cs1 ; C#Cc1nc(NC(=O)N(CCS(C)(=O)=O)Cc2ccc(-c3cccc4ncsc34)cc2)cs1]

1-(4-(Benzo[d]thiazol-7-yl)benzyl)-1-(cyanomethyl)-3-(2-ethynylthiazol-4-yl)urea A208. ¹H NMR (400 MHz, DMSO-d₆) δ 9.54-9.44 (m, 1H), 8.55 (d, J=108.8 Hz, 1H), 8.11 (t, J=7.3 Hz, 1H), 7.89 (dd, J=29.1, 6.5 Hz, 1H), 7.78-7.72 (m, 2H), 7.68 (t, J=7.6 Hz, 1H), 7.63-7.55 (m, 1H), 7.56-7.45 (m, 2H), 5.18-4.94 (m, 1H), 4.67-4.55 (m, 2H), 4.12 (dd, J=39.1, 16.3 Hz, 2H); MS (ESI) m/z: 431.1 [M+H⁺].

2-(4-(Benzo[d]thiazol-7-yl)phenyl)-2-(3-(2-ethynylthiazol-4-yl)ureido)acetamide A209. ¹H NMR (400 MHz, DMSO-d₆) δ 9.64 (s, 1H), 9.46 (s, 1H), 8.11 (dd, J=8.1, 1.0 Hz, 1H), 7.92 (s, 1H), 7.73 (d, J=8.3 Hz, 2H), 7.69-7.65 (m, 1H), 7.61-7.58 (m, 3H), 7.45 (d, J=7.7 Hz, 1H), 7.31 (s, 1H), 7.28 (s, 1H), 5.40 (d, J=7.7 Hz, 1H), 4.90 (s, 1H); MS (ESI) m/z: 434.1 [M+H⁺].

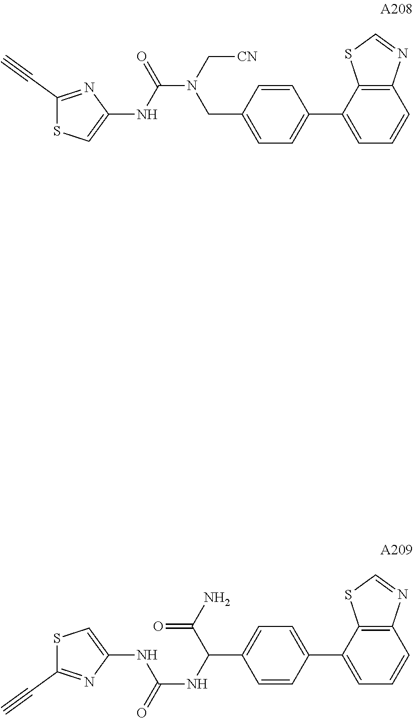 [SMILES: C#Cc1nc(NC(=O)N(CC#N)Cc2ccc(-c3cccc4ncsc34)cc2)cs1 ; C#Cc1nc(NC(=O)NC(C(N)=O)c2ccc(-c3cccc4ncsc34)cc2)cs1]

(R)-2-(4-(Benzo[d]thiazol-7-yl)phenyl)-2-(3-(2-ethynylthiazol-4-yl)ureido)-acetamide A210. ¹H NMR (400 MHz, DMSO-d₆) δ 9.64 (s, 1H), 9.64 (s, 1H), 9.46 (s, 1H), 8.11 (dd, J=8.0, 0.9 Hz, 1H), 7.92 (s, 1H), 7.73 (d, J=8.3 Hz, 2H), 7.67 (t, J=7.7 Hz, 1H), 7.61-7.55 (m, 3H), 7.45 (d, J=7.7 Hz, 1H), 7.31 (s, 1H), 7.28 (s, 1H), 5.40 (d, J=7.6 Hz, 1H), 4.90 (s, 1H); MS (ESI) m/z: 434.1 [M+H⁺].

(S)-2-(4-(Benzo[d]thiazol-7-yl)phenyl)-2-(3-(2-ethynylthiazol-4-yl)ureido)-acetamide A211. ¹H NMR (400 MHz, DMSO-d₆) δ 9.64 (s, 1H), 9.46 (s, 1H), 8.14-8.07 (m, 1H), 7.92 (s, 1H), 7.73 (d, J=8.2 Hz, 2H), 7.67 (t, J=7.7 Hz, 1H), 7.61-7.55 (m, 3H), 7.45 (d, J=7.7 Hz, 1H), 7.31 (s, 1H), 7.28 (s, 1H), 5.40 (d, J=7.6 Hz, 1H), 4.90 (s, 1H); MS (ESI) m/z: 434.1 [M+H⁺].

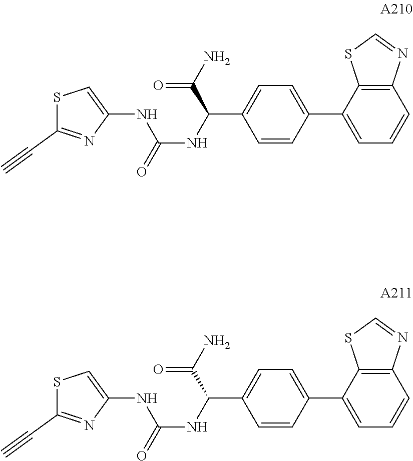 [SMILES: C#Cc1nc(NC(=O)N[C@@H](C(N)=O)c2ccc(-c3cccc4ncsc34)cc2)cs1 ; C#Cc1nc(NC(=O)N[C@H](C(N)=O)c2ccc(-c3cccc4ncsc34)cc2)cs1]

(R)-2-(3′-(1-Cyanocyclopropyl)-[1,1′-biphenyl]-4-yl)-2-(3-(2-ethynylthiazol-4-yl)ureido)acetamide A212. ¹H NMR (400 MHz, DMSO-d₆) δ 9.61 (d, J=11.4 Hz, 1H), 7.86 (s, 1H), 7.65 (t, J=6.7 Hz, 2H), 7.58 (dd, J=9.1, 1.4 Hz, 1H), 7.51 (d, J=8.1 Hz, 2H), 7.49-7.45 (m, 2H), 7.40 (d, J=7.7 Hz, 1H), 7.39-7.35 (m, 1H), 7.25 (d, J=3.3 Hz, 2H), 5.42-5.25 (m, 1H), 4.90 (s, 1H), 1.81-1.74 (m, 2H), 1.62 (q, J=5.1 Hz, 2H); MS (ESI) m/z: 442.2 [M+H⁺].

(R)-2-(3-(2-Ethynylthiazol-4-yl)ureido)-N-methyl-2-(4-(4-oxo-3,4-dihydro-quinazolin-5-yl)phenyl)acetamide A213. ¹H NMR (400 MHz, DMSO-d₆) δ 12.00 (s, 1H), 9.60 (s, 1H), 8.44 (d, J=4.1 Hz, 1H), 8.06 (s, 1H), 7.77 (t, J=7.7 Hz, 1H), 7.66 (d, J=8.0 Hz, 1H), 7.41 (d, J=7.6 Hz, 1H), 7.36 (d, J=7.7 Hz, 2H), 7.31-7.25 (m, 3H), 7.23 (d, J=7.2 Hz, 1H), 5.38 (d, J=7.9 Hz, 1H), 3.32 (s, 6H), 2.64 (d, J=3.8 Hz, 4H); MS (ESI) m/z: 459.1 [M+H⁺].

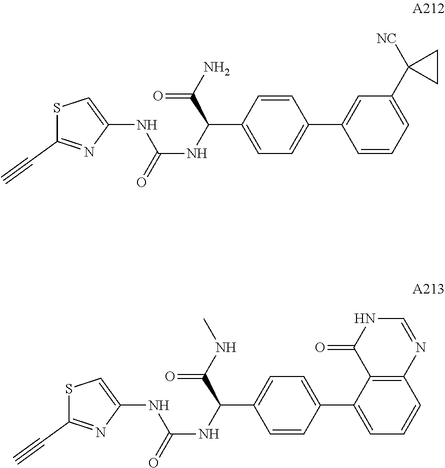 [SMILES: C#Cc1nc(NC(=O)N[C@@H](C(N)=O)c2ccc(-c3cccc(C4(C#N)CC4)c3)cc2)cs1 ; C#Cc1nc(NC(=O)N[C@@H](C(=O)NC)c2ccc(-c3cccc4nc[nH]c(=O)c34)cc2)cs1]

2-(4-(Benzo[d]thiazol-7-yl)phenyl)-2-(3-(2-ethynylthiazol-4-yl)ureido)-N-methylacetamide A214. ¹H NMR (400 MHz, DMSO-d₆) δ 9.61 (s, 1H), 9.46 (s, 1H), 8.46 (d, J=4.7 Hz, 1H), 8.11 (dd, J=8.1, 1.0 Hz, 1H), 7.73 (d, J=8.3 Hz, 2H), 7.70-7.62 (m, 1H), 7.57 (t, J=7.9 Hz, 3H), 7.48 (d, J=7.8 Hz, 1H), 7.28 (s, 1H), 5.41 (d, J=7.7 Hz, 1H), 4.90 (s, 1H), 2.64 (d, J=4.6 Hz, 3H); MS (ESI) m/z: 448.1 [M+H⁺].

(R)-2-(4-(Benzo[d]thiazol-7-yl)phenyl)-2-(3-(2-ethynylthiazol-4-yl)ureido)-N-methylacetamide A215. ¹H NMR (400 MHz, DMSO-d₆) δ 9.61 (s, 1H), 9.46 (s, 1H), 8.46 (q, J=4.4 Hz, 1H), 8.11 (dd, J=8.0, 0.9 Hz, 1H), 7.73 (d, J=8.3 Hz, 2H), 7.67 (t, J=7.8 Hz, 1H), 7.57 (t, J=7.7 Hz, 3H), 7.48 (d, J=7.8 Hz, 1H), 7.28 (s, 1H), 5.41 (d, J=7.8 Hz, 1H), 4.90 (s, 1H), 2.64 (d, J=4.6 Hz, 3H); MS (ESI) m/z: 448.1 [M+H⁺].

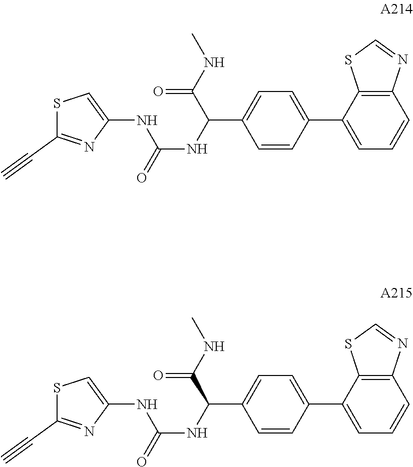 [SMILES: C#Cc1nc(NC(=O)NC(C(=O)NC)c2ccc(-c3cccc4ncsc34)cc2)cs1 ; C#Cc1nc(NC(=O)N[C@@H](C(=O)NC)c2ccc(-c3cccc4ncsc34)cc2)cs1]

(S)-2-(4-(Benzo[d]thiazol-7-yl)phenyl)-2-(3-(2-ethynylthiazol-4-yl)ureido)-N-methylacetamide A216. ¹H NMR (400 MHz, DMSO-d₆) δ 9.62 (s, 1H), 9.46 (s, 1H), 8.47 (q, J=4.4 Hz, 1H), 8.11 (dd, J=8.0, 0.9 Hz, 1H), 7.73 (d, J=8.3 Hz, 2H), 7.67 (t, J=7.8 Hz, 1H), 7.58 (t, J=7.2 Hz, 3H), 7.48 (d, J=7.8 Hz, 1H), 7.29 (d, J=4.0 Hz, 1H), 5.41 (d, J=7.8 Hz, 1H), 4.90 (s, 1H), 2.64 (d, J=4.6 Hz, 3H); MS (ESI) m/z: 448.1 [M+H⁺].

(R)-2-(3′-(1-Cyanocyclopropyl)-[1,1′-biphenyl]-4-yl)-2-(3-(2-ethynylthiazol-4-yl)ureido)-N-methylacetamide A217. ¹H NMR (400 MHz, DMSO-d₆) δ 9.61 (s, 1H), 8.56-8.27 (m, 1H), 7.65 (d, J=8.2 Hz, 2H), 7.57 (d, J=7.8 Hz, 1H), 7.51-7.41 (m, 5H), 7.37 (d, J=7.8 Hz, 1H), 7.26 (s, 1H), 5.35 (d, J=7.8 Hz, 1H), 4.90 (s, 1H), 2.61 (d, J=4.5 Hz, 3H), 1.91-1.71 (m, 2H), 1.65-1.52 (m, 2H); MS (ESI) m/z: 456.2 [M+H⁺].

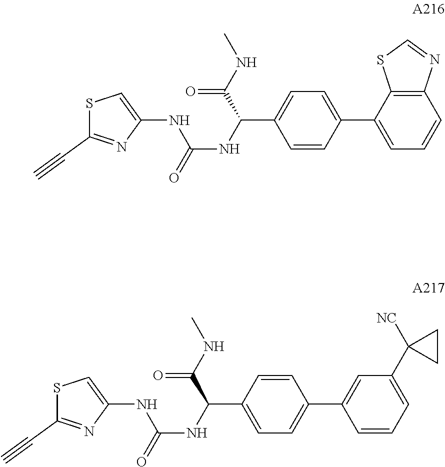 [SMILES: C#Cc1nc(NC(=O)N[C@H](C(=O)NC)c2ccc(-c3cccc4ncsc34)cc2)cs1 ; C#Cc1nc(NC(=O)N[C@@H](C(=O)NC)c2ccc(-c3cccc(C4(C#N)CC4)c3)cc2)cs1]

(R)-2-(3-(2-Ethynylthiazol-4-yl)ureido)-2-(3′-(1-hydroxycyclopropyl)-[1,1′-biphenyl]-4-yl)-N-methylacetamide A218. ¹H NMR (400 MHz, DMSO-d₆) δ 9.60 (s, 1H), 8.39 (d, J=4.7 Hz, 1H), 7.62 (d, J=8.3 Hz, 2H), 7.49-7.44 (m, 3H), 7.42 (dd, J=7.7, 1.1 Hz, 2H), 7.36 (t, J=7.6 Hz, 1H), 7.26 (s, 1H), 7.24-7.19 (m, 1H), 5.96 (s, 1H), 5.34 (d, J=7.8 Hz, 1H), 4.90 (s, 1H), 2.61 (d, J=4.6 Hz, 3H), 1.12 (dd, J=7.1, 4.7 Hz, 2H), 1.02 (dd, J=7.2, 4.9 Hz, 2H); MS (ESI) m/z: 448.1 [M+H⁺].

(R)-2-(3-(2-Ethynylthiazol-4-yl)ureido)-N-methyl-2-(3′-propionyl-[1,1′-biphenyl]-4-yl)acetamide A219. ¹H NMR (400 MHz, DMSO-d₆) δ 9.63 (s, 1H), 8.42 (d, J=4.7 Hz, 1H), 8.18 (t, J=1.6 Hz, 1H), 7.98-7.86 (m, 2H), 7.72 (d, J=8.3 Hz, 2H), 7.61 (t, J=7.7 Hz, 1H), 7.49 (dd, J=14.8, 8.0 Hz, 3H), 7.26 (s, 1H), 5.37 (d, J=7.8 Hz, 1H), 4.90 (s, 1H), 3.13 (q, J=7.2 Hz, 2H), 2.61 (d, J=4.6 Hz, 3H), 1.11 (t, J=7.2 Hz, 3H); MS (ESI) m/z: 448.1 [M+H⁺].

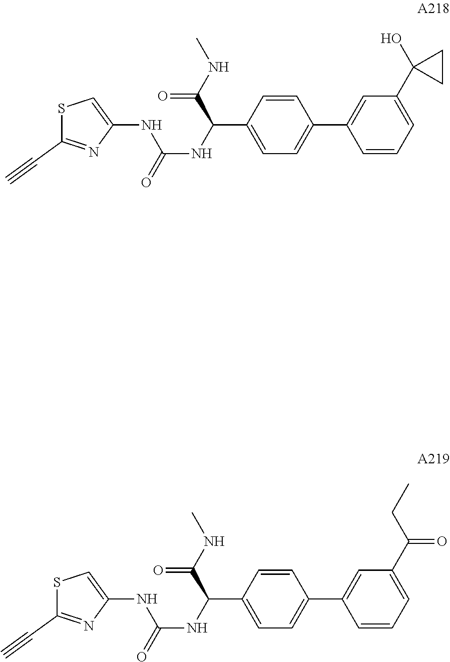 [SMILES: C#Cc1nc(NC(=O)N[C@@H](C(=O)NC)c2ccc(-c3cccc(C4(O)CC4)c3)cc2)cs1 ; C#Cc1nc(NC(=O)N[C@@H](C(=O)NC)c2ccc(-c3cccc(C(=O)CC)c3)cc2)cs1]

(R)-2-(4-(Benzo[d]thiazol-7-yl)phenyl)-2-(3-(2-ethynylthiazol-4-yl)ureido)-N,N-dimethylacetamide A220. ¹H NMR (400 MHz, DMSO-d₆) δ 9.63 (s, 1H), 9.46 (s, 1H), 8.11 (d, J=8.0 Hz, 1H), 7.76 (d, J=8.2 Hz, 2H), 7.67 (t, J=7.8 Hz, 1H), 7.60 (d, J=7.7 Hz, 1H), 7.56 (s, 2H), 7.43 (d, J=7.6 Hz, 1H), 7.30 (s, 1H), 5.84 (d, J=7.7 Hz, 1H), 4.90 (s, 1H), 3.03 (s, 3H), 2.90 (s, 3H); MS (ESI) m/z: 462.1 [M+H⁺].

(R)-2-(3′-Cyano-[1,1′-biphenyl]-4-yl)-2-(3-(2-ethynylthiazol-4-yl)ureido)ethyl carbamate A221. ¹H NMR (400 MHz, DMSO-d₆) δ 9.52 (s, 1H), 8.16 (s, 1H), 8.02 (d, J=8.0 Hz, 1H), 7.82 (d, J=7.7 Hz, 1H), 7.75 (d, J=8.3 Hz, 2H), 7.67 (t, J=7.8 Hz, 1H), 7.48 (d, J=8.3 Hz, 2H), 7.28 (s, 1H), 7.09 (d, J=8.1 Hz, 1H), 6.58 (s, 2H), 5.05 (dd, J=12.7, 7.3 Hz, 1H), 4.90 (s, 1H), 4.17 (qd, J=11.3, 6.2 Hz 2H); MS (ESI) m/z: 432.1 [M+H⁺].

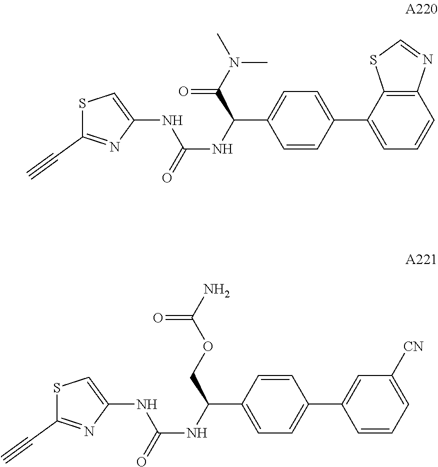 [SMILES: C#Cc1nc(NC(=O)N[C@@H](C(=O)N(C)C)c2ccc(-c3cccc4ncsc34)cc2)cs1 ; C#Cc1nc(NC(=O)N[C@@H](COC(N)=O)c2ccc(-c3cccc(C#N)c3)cc2)cs1]

(R)-2-(3′-(1-Cyanocyclopropyl)-[1,1′-biphenyl]-4-yl)-2-(3-(2-ethynylthiazol-4-yl)ureido)ethyl carbonate A222. ¹H NMR (400 MHz, DMSO-d₆) δ 9.52 (s, 1H), 7.67 (d, J=8.3 Hz, 2H), 7.59 (d, J=8.1 Hz, 1H), 7.53-7.39 (m, 4H), 7.38-7.33 (m, 1H), 7.28 (s, 1H), 7.08 (d, J=8.2 Hz, 1H), 6.57 (s, 2H), 5.04 (dd, J=12.9, 7.7 Hz, 1H), 4.90 (s, 1H), 4.16 (qd, J=11.3, 6.2 Hz, 2H), 1.77 (q, J=4.7 Hz, 2H), 1.62 (q, J=5.1 Hz, 2H).

(R)-2-(4-(Benzo[d]thiazol-7-yl)phenyl)-2-(3-(2-ethynylthiazol-4-yl)ureido)ethyl carbamate A223. ¹H NMR (400 MHz, DMSO-d₆) δ 9.52 (s, 1H), 9.46 (s, 1H), 8.11 (dd, J=8.0, 0.9 Hz, 1H), 7.73 (d, J=8.2 Hz, 2H), 7.67 (t, J=7.8 Hz, 1H), 7.59 (d, J=6.9 Hz, 1H), 7.55 (d, J=8.2 Hz, 2H), 7.30 (s, 1H), 7.10 (d, J=8.0 Hz, 1H), 6.59 (s, 2H), 5.09 (dd, J=12.7, 7.3 Hz, 1H), 4.90 (s, 1H), 4.21 (ddd, J=18.6, 11.3, 6.2 Hz, 2H); MS (ESI) m/z: 464.1 [M+H⁺].

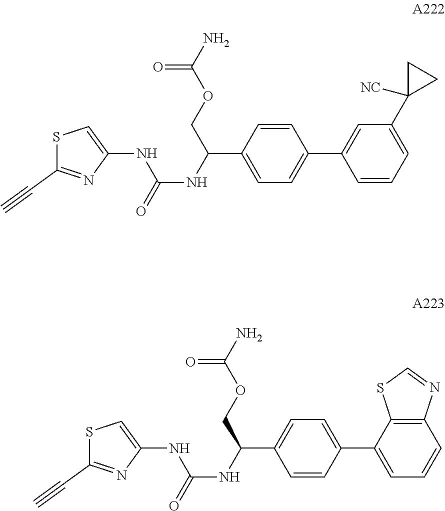 [SMILES: C#Cc1nc(NC(=O)NC(COC(N)=O)c2ccc(-c3cccc(C4(C#N)CC4)c3)cc2)cs1 ; C#Cc1nc(NC(=O)N[C@@H](COC(N)=O)c2ccc(-c3cccc4ncsc34)cc2)cs1]

(R)-2-(4-(Benzo[d]thiazol-7-yl)phenyl)-2-(3-(2-ethynylthiazol-4-yl)ureido)ethyl methylcarbamate A224. ¹H NMR (400 MHz, DMSO-d₆) δ 9.53 (s, 1H), 9.46 (s, 1H), 8.12 (d, J=8.0 Hz, 1H), 7.74 (d, J=8.0 Hz, 2H), 7.68 (t, J=7.7 Hz, 1H), 7.58 (dd, J=15.9, 7.7 Hz, 3H), 7.31 (s, 1H), 7.23-7.04 (m, 2H), 5.10 (d, J=5.2 Hz, 1H), 4.90 (s, 1H), 4.36-4.14 (m, 2H), 2.59 (d, J=4.5 Hz, 3H); MS (ESI) m/z: 478.1 [M+H⁺].

(R)-2-(4-(Benzo[d][1,2,3]thiadiazol-7-yl)phenyl)-2-(3-(2-ethynylthiazol-4-yl)-ureido)ethyl carbamate A225. ¹H NMR (400 MHz, DMSO-d₆) δ 9.57 (s, 1H), 8.75 (dd, J=8.3, 0.7 Hz, 1H), 8.04-7.97 (m, 1H), 7.94-7.87 (m, 1H), 7.78 (d, J=8.3 Hz, 2H), 7.58 (d, J=8.2 Hz, 2H), 7.29 (s, 1H), 7.15 (d, J=8.0 Hz, 1H), 6.59 (s, 2H), 5.10 (dd, J=12.7, 7.4 Hz, 1H), 4.90 (s, 1H), 4.22 (ddd, J=18.5, 11.3, 6.1 Hz, 2H); MS (ESI) m/z: 465.1 [M+H⁺].

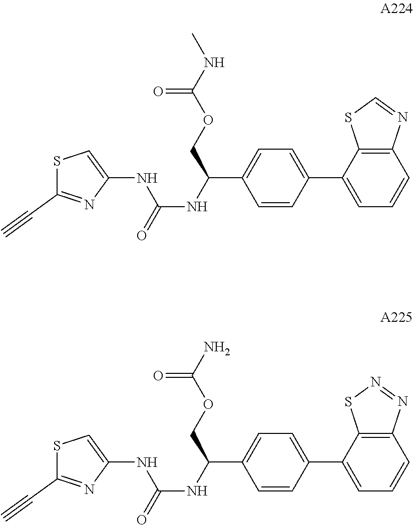 [SMILES: C#Cc1nc(NC(=O)N[C@@H](COC(=O)NC)c2ccc(-c3cccc4ncsc34)cc2)cs1 ; C#Cc1nc(NC(=O)N[C@@H](COC(N)=O)c2ccc(-c3cccc4nnsc34)cc2)cs1]

(R)-2-(4-(Benzo[c][1,2,5]thiadiazol-4-yl)phenyl)-2-(3-(2-ethynylthiazol-4-yl)-ureido)ethyl carbamate A226. ¹H NMR (400 MHz, DMSO-d₆) δ 9.53 (s, 1H), 8.19-8.05 (m, 1H), 7.96 (d, J=8.3 Hz, 2H), 7.84 (dd, J=10.4, 4.9 Hz, 2H), 7.53 (d, J=8.3 Hz, 2H), 7.30 (s, 1H), 7.11 (d, J=8.2 Hz, 1H), 6.60 (s, 2H), 5.09 (dd, J=12.8, 7.4 Hz, 1H), 4.90 (s, 1H), 4.22 (qd, J=11.3, 6.2 Hz, 2H); MS (ESI) m/z: 465.0 [M+H⁺].

(R)-2-(3-(2-Ethynylthiazol-4-yl)ureido)-2-(4-(quinolin-8-yl)phenyl)ethyl carbamate A227. ¹H NMR (400 MHz, DMSO-d₆) δ 9.52 (s, 1H), 8.90 (dd, J=4.1, 1.8 Hz, 1H), 8.43 (dd, J=8.3, 1.8 Hz, 1H), 8.00 (dd, J=8.1, 1.4 Hz, 1H), 7.77 (dd, J=7.2, 1.5 Hz, 1H), 7.69 (d, J=8.0 Hz, 1H), 7.67-7.61 (m, 2H), 7.57 (dd, J=8.3, 4.1 Hz, 1H), 7.46 (d, J=8.2 Hz, 2H), 7.31 (s, 1H), 7.08 (d, J=8.2 Hz, 1H), 6.65 (s, 2H), 5.08 (dd, J=12.9, 7.5 Hz, 1H), 4.90 (s, 1H), 4.23 (ddd, J=18.6, 11.2, 6.2 Hz, 2H); MS (ESI) m/z: 458.1 [M+H⁺].

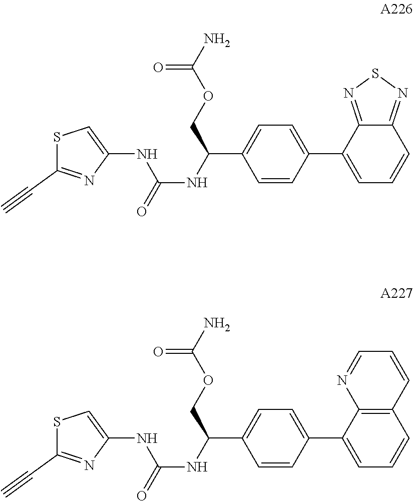 [SMILES: C#Cc1nc(NC(=O)N[C@@H](COC(N)=O)c2ccc(-c3cccc4nsnc34)cc2)cs1 ; C#Cc1nc(NC(=O)N[C@@H](COC(N)=O)c2ccc(-c3cccc4cccnc34)cc2)cs1]

(R)-2-(3-(2-Ethynylthiazol-4-yl)ureido)-2-(4-(isoquinolin-8-yl)phenyl)ethyl carbamate A228. ¹H NMR (400 MHz, DMSO-d₆) δ 9.55 (s, 1H), 9.17 (s, 1H), 8.54 (d, J=5.7 Hz, 1H), 8.01 (d, J=8.3 Hz, 1H), 7.92 (d, J=5.7 Hz, 1H), 7.87-7.81 (m, 1H), 7.63-7.53 (m, 5H), 7.31 (s, 1H), 7.14 (d, J=8.2 Hz, 1H), 6.61 (s, 2H), 5.19-5.07 (m, 1H), 4.90 (s, 1H), 4.28 (dd, J=11.3, 4.9 Hz, 1H), 4.21 (dd, J=11.2, 7.4 Hz, 1H); MS (ESI) m/z: 458.2 [M+H⁺].

(R)-2-(3-(2-Ethynylthiazol-4-yl)ureido)-2-(4-(isoquinolin-5-yl)phenyl)ethyl carbamate A229. ¹H NMR (400 MHz, DMSO-d₆) δ 9.53 (s, 1H), 9.39 (s, 1H), 8.49 (d, J=6.0 Hz, 1H), 8.17 (dd, J=7.6, 1.1 Hz, 1H), 7.81-7.71 (m, 2H), 7.68 (d, J=6.0 Hz, 1H), 7.59-7.49 (m, 4H), 7.31 (s, 1H), 7.12 (d, J=8.2 Hz, 1H), 6.60 (s, 2H), 5.17-5.06 (m, 1H), 4.90 (s, 1H), 4.26 (dd, J=11.2, 4.9 Hz, 1H), 4.19 (dd, J=11.2, 7.5 Hz, 1H); MS (ESI) m/z: 458.1 [M+H⁺].

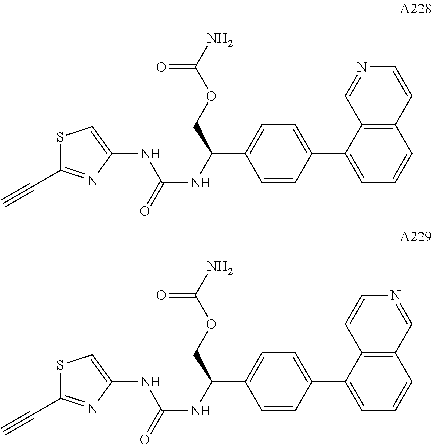 [SMILES: C#Cc1nc(NC(=O)N[C@@H](COC(N)=O)c2ccc(-c3cccc4ccncc34)cc2)cs1 ; C#Cc1nc(NC(=O)N[C@@H](COC(N)=O)c2ccc(-c3cccc4cnccc34)cc2)cs1]

(R)-2-(3-(2-Ethynylthiazol-4-yl)ureido)-2-(4-(quinolin-5-yl)phenyl)ethyl carbamate A230. ¹H NMR (400 MHz, DMSO-d₆) δ 9.54 (s, 1H), 8.94 (dd, J=4.1, 1.6 Hz, 1H), 8.21 (d, J=8.0 Hz, 1H), 8.06 (d, J=8.5 Hz, 1H), 7.83 (dd, J=8.5, 7.2 Hz, 1H), 7.59-7.47 (m, 6H), 7.31 (s, 1H), 7.13 (d, J=8.3 Hz, 1H), 6.58 (s, 2H), 5.17-5.07 (m, 1H), 4.90 (s, 1H), 4.26 (dd, J=11.3, 4.9 Hz, 1H), 4.19 (dd, J=11.2, 7.5 Hz, 1H); MS (ESI) m/z: 458.1 [M+H⁺].

(R)-2-(3-(2-Ethynylthiazol-4-yl)ureido)-2-(4-(1-methyl-1H-indazol-4-yl)phenyl)-ethyl carbamate A231. ¹H NMR (400 MHz, DMSO-d₆) δ 9.66 (s, 1H), 8.15 (s, 1H), 7.74 (d, J=8.2 Hz, 2H), 7.64 (d, J=8.4 Hz, 1H), 7.55-7.50 (m, 2H), 7.49-7.45 (m, 1H), 7.29 (s, 1H), 7.26 (d, J=7.3 Hz, 1H), 7.10 (d, J=8.2 Hz, 1H), 6.60 (s, 1H), 5.08 (dd, J=12.8, 7.8 Hz, 1H), 4.92 (s, 1H), 4.20 (ddd, J=18.7, 11.3, 6.2 Hz, 2H), 4.09 (s, 3H); MS (ESI) m/z: 461.1 [M+H⁺].

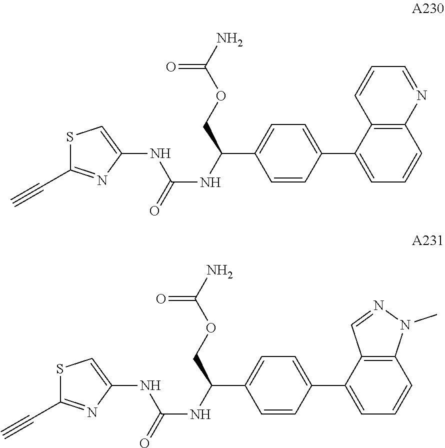 [SMILES: C#Cc1nc(NC(=O)N[C@@H](COC(N)=O)c2ccc(-c3cccc4ncccc34)cc2)cs1 ; C#Cc1nc(NC(=O)N[C@@H](COC(N)=O)c2ccc(-c3cccc4c3cnn4C)cc2)cs1]

(2R)-2-(3-(2-Ethynylthiazol-4-yl)ureido)-2-(4-(1-methyl-4,5,6,7-tetrahydro-1H-indazol-4-yl)phenyl)ethyl carbamate A232. ¹H NMR (400 MHz, DMSO-d₆) δ 9.44 (d, J=2.9 Hz, 1H), 7.26 (d, J=7.9 Hz, 3H), 7.13 (d, J=8.2 Hz, 2H), 6.98 (d, J=6.7 Hz, 1H), 6.87 (d, J=2.6 Hz, 1H), 6.57 (s, 2H), 4.96 (dd, J=13.0, 7.7 Hz, 1H), 4.89 (s, 1H), 4.14 (dd, J=11.2, 5.0 Hz, 1H), 4.11-4.03 (m, 1H), 3.92-3.86 (m, 1H), 3.69 (s, 3H), 2.66-2.60 (m, 2H), 1.97 (dt, J=12.1, 4.9 Hz, 1H), 1.86 (d, J=5.5 Hz, 1H), 1.69 (t, J=14.0 Hz, 1H), 1.55 (dt, J=18.7, 9.4 Hz, 1H); MS (ESI) m/z: 465.3 [M+H⁺].

(R)-2-(3-(2-Ethynylthiazol-4-yl)ureido)-2-(4-(1-methyl-6,7-dihydro-1H-indazol-4-yl)phenyl)ethyl carbamate A233. ¹H NMR (400 MHz, DMSO-d₆) δ 9.49 (s, 1H), 7.45 (d, J=8.3 Hz, 2H), 7.37 (d, J=8.3 Hz, 2H), 7.28 (d, J=5.1 Hz, 2H), 7.05 (d, J=8.2 Hz, 1H), 6.57 (s, 2H), 5.72 (t, J=4.5 Hz, 1H), 5.01 (dd, J=12.8, 7.7 Hz, 1H), 4.89 (s, 1H), 4.14 (ddd, J=18.8, 11.3, 6.3 Hz, 2H), 3.75 (s, 3H), 2.79 (t, J=8.7 Hz, 2H); MS (ESI) m/z: 463.2 [M+H⁺].

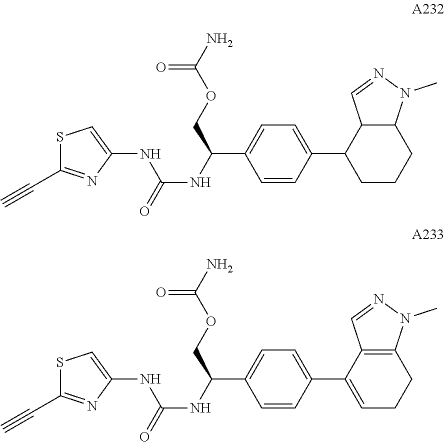

(R)-2-(3-(2-Ethynylthiazol-4-yl)ureido)-2-(4-(6-(pyrrolidin-1-yl)pyridin-2-yl)-phenyl)ethyl carbamate A234. ¹H NMR (400 MHz, DMSO-d₆) δ 9.49 (s, 1H), 8.02 (d, J=8.4 Hz, 2H), 7.55 (dd, J=8.3, 7.5 Hz, 1H), 7.41 (d, J=8.3 Hz, 2H), 7.28 (s, 1H), 7.07 (dd, J=14.9, 7.8 Hz, 2H), 6.57 (s, 2H), 6.40 (d, J=8.3 Hz, 1H), 5.07-4.96 (m, 1H), 4.89 (d, J=1.6 Hz, 1H), 4.16 (ddd, J=18.6, 11.2, 6.2 Hz, 2H), 3.46 (t, J=6.5 Hz, 5H), 2.07-1.86 (m, 4H); MS (ESI) m/z: 477.2 [M+H⁺].

(R)-2-(3-(2-Ethynylthiazol-4-yl)ureido)-2-(4-(2-(pyrrolidin-1-yl)pyridin-4-yl)-phenyl)ethyl carbamate A235. ¹H NMR (400 MHz, DMSO-d₆) δ 9.51 (s, 1H), 8.10 (d, J=5.3 Hz, 1H), 7.71 (d, J=8.3 Hz, 2H), 7.46 (d, J=8.3 Hz, 2H), 7.27 (s, 1H), 7.08 (d, J=8.2 Hz, 1H), 6.81 (dd, J=5.3, 1.3 Hz, 1H), 6.62 (s, 2H), 5.10-4.96 (m, 1H), 4.90 (s, 1H), 4.16 (qd, J=11.3, 6.2 Hz, 2H), 3.44 (t, J=6.5 Hz, 5H), 1.95 (t, J=6.6 Hz, 4H).

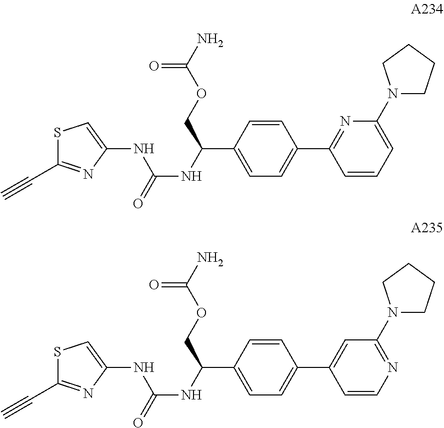 [SMILES: C#Cc1nc(NC(=O)N[C@@H](COC(N)=O)c2ccc(-c3cccc(N4CCCC4)n3)cc2)cs1 ; C#Cc1nc(NC(=O)N[C@@H](COC(N)=O)c2ccc(-c3ccnc(N4CCCC4)c3)cc2)cs1]

(R)-2-(5-(3-(1-Cyanocyclopropyl)phenyl)pyridin-2-yl)-2-(3-(2-ethynylthiazol-4-yl)ureido)ethyl carbamate A236. ¹H NMR (400 MHz, DMSO-d₆) δ 9.65 (s, 1H), 8.89 (d, J=1.9 Hz, 1H), 8.11 (dd, J=8.1, 2.4 Hz, 1H), 7.70-7.62 (m, 1H), 7.58-7.43 (m, 4H), 7.30 (s, 1H), 7.14 (d, J=8.3 Hz, 1H), 6.54 (s, 2H), 5.14 (dd, J=14.2, 6.1 Hz, 1H), 4.90 (s, 1H), 4.27 (d, J=6.3 Hz, 2H), 1.78 (dd, J=7.7, 4.7 Hz, 2H), 1.66 (dd, J=8.0, 5.2 Hz, 2H); MS (ESI) m/z: 473.1 [M+H⁺].

1-(1-(4-(Benzo[d]thiazol-7-yl)phenyl)piperidin-4-yl)-3-(2-ethynylthiazol-4-yl)urea A237. ¹H NMR (400 MHz, DMSO-d₆) δ 9.43 (s, 1H), 9.22 (s, 1H), 8.03 (dd, J=8.0, 0.9 Hz, 1H), 7.66-7.56 (m, 3H), 7.53 (d, J=6.9 Hz, 1H), 7.31 (s, 1H), 7.12 (d, J=8.9 Hz, 2H), 6.46 (d, J=7.5 Hz, 1H), 4.89 (s, 1H), 3.72 (d, J=13.1 Hz, 3H), 2.99 (t, J=10.5 Hz, 2H), 1.95 (d, J=9.8 Hz, 2H), 1.51 (dd, J=19.6, 9.9 Hz, 2H); MS (ESI) m/z: 460.1 [M+H⁺].

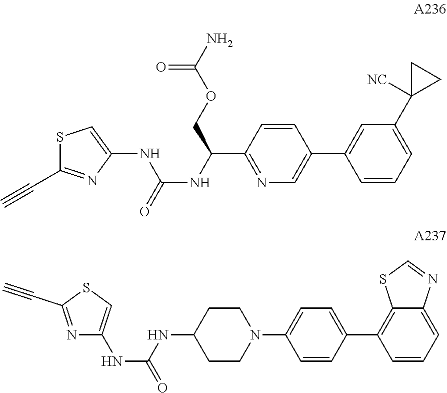 [SMILES: C#Cc1nc(NC(=O)N[C@@H](COC(N)=O)c2ccc(-c3cccc(C4(C#N)CC4)c3)cn2)cs1 ; C#Cc1nc(NC(=O)NC2CCN(c3ccc(-c4cccc5ncsc45)cc3)CC2)cs1]

1-((4-(3-(1-Cyanocyclopropyl)phenyl)cyclohexyl)methyl)-3-(2-ethynylthiazol-4-yl)urea A238. ¹H NMR (400 MHz, DMSO-d₆) δ 9.30 (d, J=3.7 Hz, 1H), 7.35-7.25 (m, 2H), 7.23-7.10 (m, 3H), 6.42 (dt, J=11.5, 5.7 Hz, 1H), 4.89 (d, J=1.1 Hz, 1H), 3.27-3.18 (m, 1H), 3.02 (t, J=6.1 Hz, 1H), 2.08 (s, 1H), 1.87-1.39 (m, 12H), 1.15-1.01 (m, 1H); MS (ESI) m/z: 405.3 [M+H⁺].

1-((3′-(1-Cyanocyclopropyl)-2,3,4,5-tetrahydro-[1,1′-biphenyl]-4-yl)methyl)-3-(2-ethynylthiazol-4-yl)urea A239. ¹H NMR (400 MHz, DMSO-d₆) δ 9.31 (s, 1H), 7.37-7.31 (m, 2H), 7.29 (s, 2H), 7.25-7.14 (m, 1H), 6.48 (t, J=5.7 Hz, 1H), 6.17 (s, 1H), 4.89 (s, 1H), 3.11 (t, J=6.2 Hz, 2H), 2.44 (s, 2H), 2.35-2.23 (m, 1H), 1.88 (d, J=11.0 Hz, 2H), 1.72 (dd, J=7.8, 4.9 Hz, 3H), 1.52 (q, J=4.9 Hz, 2H), 1.42-1.27 (m, 1H); MS (ESI) m/z: 403.1 [M+H⁺].

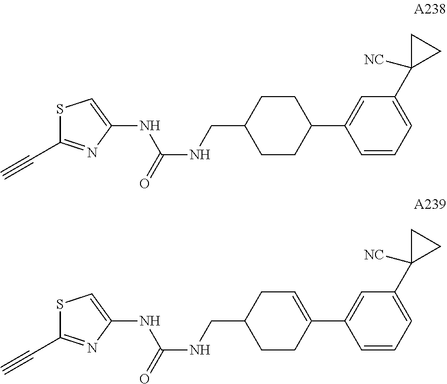 [SMILES: C#Cc1nc(NC(=O)NCC2CCC(c3cccc(C4(C#N)CC4)c3)CC2)cs1 ; C#Cc1nc(NC(=O)NCC2CC=C(c3cccc(C4(C#N)CC4)c3)CC2)cs1]

1-((1-(3-(1-Cyanocyclopropyl)phenyl)piperidin-4-yl)methyl)-3-(2-ethynylthiazol-4-yl)urea A240. ¹H NMR (400 MHz, DMSO-d₆) δ 9.31 (s, 1H), 7.28 (s, 1H), 7.19 (t, J=8.0 Hz, 1H), 6.87 (dd, J=8.1, 2.1 Hz, 1H), 6.79 (s, 1H), 6.71 (d, J=7.6 Hz, 1H), 6.44 (t, J=5.8 Hz, 1H), 3.72 (d, J=12.9 Hz, 2H), 3.05 (t, J=6.2 Hz, 2H), 2.64 (d, J=10.0 Hz, 2H), 1.72 (d, J=11.7 Hz, 2H), 1.67 (dd, J=7.6, 4.7 Hz, 2H), 1.58 (s, 1H), 1.47 (q, J=5.0 Hz, 2H), 1.24 (dd, J=21.1, 12.1 Hz, 2H); MS (ESI) m/z: 406.2 [M+H⁺].

(R)-1-(1-(4-(Benzo[d]thiazol-7-yl)phenyl)-2-hydroxyethyl)-3-(2-ethynyl-pyrimidin-4-yl)urea A241. ¹H NMR (400 MHz, DMSO-d₆) δ 9.85 (s, 1H), 9.46 (s, 1H), 8.46 (d, J=5.9 Hz, 1H), 8.10 (dd, J=8.1, 1.0 Hz, 1H), 8.02 (d, J=7.0 Hz, 1H), 7.71 (d, J=8.3 Hz, 2H), 7.69-7.63 (m, 2H), 7.58 (d, J=6.8 Hz, 1H), 7.52 (d, J=8.2 Hz, 2H), 5.13 (t, J=5.1 Hz, 1H), 4.90 (dt, J=10.1, 5.0 Hz, 1H), 4.32 (s, 1H), 3.72 (dtd, J=21.4, 10.7, 5.1 Hz, 2H).

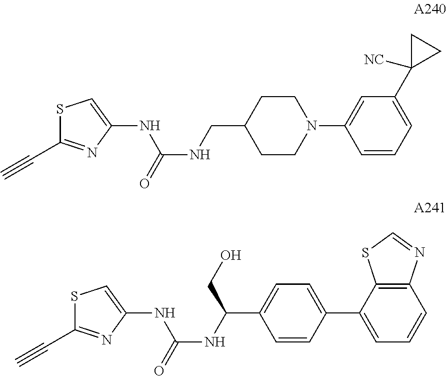 [SMILES: C#Cc1nc(NC(=O)NCC2CCN(c3cccc(C4(C#N)CC4)c3)CC2)cs1 ; C#Cc1nc(NC(=O)N[C@@H](CO)c2ccc(-c3cccc4ncsc34)cc2)cs1]

2-(4-(Benzo[d]thiazol-7-yl)phenyl)-N-(2-ethynylthiazol-4-yl)pyrrolidine-1-carboxamide B1. ¹H NMR (400 MHz, DMSO-d₆) δ 9.67-9.28 (m, 2H), 8.09 (dd, J=8.0, 1.0 Hz, 1H), 7.73-7.62 (m, 3H), 7.61-7.55 (m, 1H), 7.41-7.33 (m, 3H), 5.20 (s, 1H), 4.88 (s, 1H), 3.92-3.76 (m, 1H), 3.66-3.50 (m, 1H), 2.39-2.24 (m, 1H), 2.01-1.77 (m, 3H); MS (ESI) m/z: 431.1 [M+H⁺].

3-(4-(Benzo[d]thiazol-7-yl)phenyl)-N-(2-ethynylthiazol-4-yl)azetidine-1-carboxamide B2. ¹H NMR (400 MHz, DMSO-d₆) δ 9.91 (s, 1H), 9.46 (s, 1H), 8.11 (dd, J=8.0, 1.0 Hz, 1H), 7.73 (t, J=6.4 Hz, 2H), 7.70-7.64 (m, 1H), 7.61-7.54 (m, 3H), 7.44 (s, 1H), 4.90 (s, 1H), 4.43 (t, J=8.5 Hz, 2H), 4.03 (dd, J=8.2, 6.2 Hz, 2H), 3.92 (dt, J=8.7, 7.3 Hz, 1H); MS (ESI) m/z: 417.1 [M+H⁺].

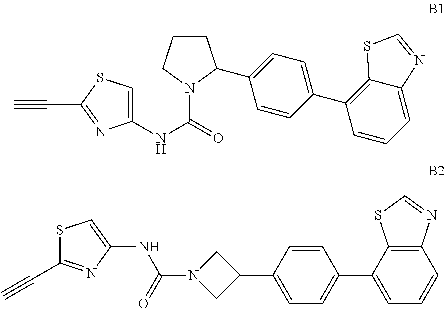 [SMILES: C#Cc1nc(NC(=O)N2CCCC2c2ccc(-c3cccc4ncsc34)cc2)cs1 ; C#Cc1nc(NC(=O)N2CC(c3ccc(-c4cccc5ncsc45)cc3)C2)cs1]

6-(Benzo[d]thiazol-7-yl)-N-(2-ethynylthiazol-4-yl)-3,4-dihydroisoquinoline-2(1H)-carboxamide B3. ¹H NMR (400 MHz, DMSO-d₆) δ 9.92 (s, 1H), 9.45 (s, 1H), 8.19-7.99 (m, 1H), 7.66 (d, J=8.0 Hz, 1H), 7.59-7.51 (m, 3H), 7.47 (s, 1H), 7.36 (s, 1H), 4.91 (s, 1H), 4.74 (s, 2H), 3.78 (t, J=5.9 Hz, 2H), 2.94 (t, J=5.7 Hz, 2H); MS (ESI) m/z: 417.2 [M+H⁺].

5-(Benzo[d]thiazol-7-yl)-N-(2-ethynylthiazol-4-yl)isoindoline-2-carboxamide B4. ¹H NMR (400 MHz, DMSO-d₆) δ 9.46 (s, 1H), 8.12 (dd, J=8.0.0.8 Hz, 1H), 7.68 (t, J=7.7 Hz, 3H), 7.60 (d, J=7.0 Hz, 1H), 7.54 (d, J=7.9 Hz, 1H), 7.52 (s, 1H), 4.92 (s, 1H), 4.86 (s, 4H); MS (ESI) m/z: 403.1 [M+H⁺].

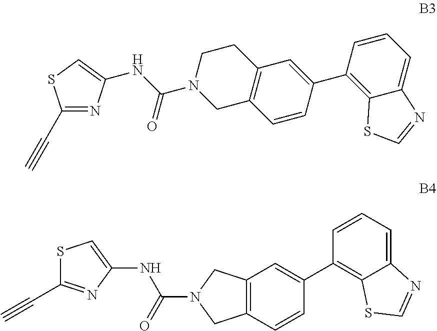 [SMILES: C#Cc1nc(NC(=O)N2CCc3cc(-c4cccc5ncsc45)ccc3C2)cs1 ; C#Cc1nc(NC(=O)N2Cc3ccc(-c4cccc5ncsc45)cc3C2)cs1]

4-(4-(Benzo[d]thiazol-7-yl)phenyl)-N-(2-ethynylthiazol-4-yl)piperazine-1-carboxamide B5. ¹H NMR (400 MHz, DMSO-d₆) δ 9.93 (s, 1H), 9.43 (s, 1H), 8.04 (dd, J=8.0, 1.0 Hz, 1H), 7.67-7.58 (m, 3H), 7.54 (d, J=6.7 Hz, 1H), 7.46 (s, 1H), 7.15 (d, J=8.9 Hz, 2H), 4.90 (s, 1H), 3.72-3.60 (m, 4H), 3.29-3.23 (m, 4H); MS (ESI) m/z: 446.2 [M+H⁺].

4-(4-(Benzo[d]thiazol-7-yl)phenyl)-N-(2-ethynylthiazol-4-yl)piperidine-1-carboxamide B6. ¹H NMR (400 MHz, DMSO-d₆) δ 9.82 (s, 1H), 9.45 (s, 1H), 8.09 (dd, J=8.0, 1.1 Hz, 1H), 7.66 (t, J=7.5 Hz, 3H), 7.58 (d, J=6.8 Hz, 1H), 7.45 (t, J=4.1 Hz, 3H), 4.90 (s, 1H), 4.34 (d, J=13.5 Hz, 2H), 3.08-2.77 (m, 3H), 1.86 (d, J=10.8 Hz, 2H), 1.74-1.49 (m, 2H); MS (ESI) m/z: 445.1 [M+H⁺].

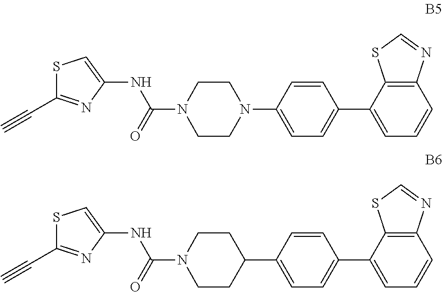 [SMILES: C#Cc1nc(NC(=O)N2CCN(c3ccc(-c4cccc5ncsc45)cc3)CC2)cs1 ; C#Cc1nc(NC(=O)N2CCC(c3ccc(-c4cccc5ncsc45)cc3)CC2)cs1]

N-(2-Ethynylthiazol-4-yl)-4-(5-(3-(2-oxooxazolidin-3-yl)phenyl)pyridin-2-yl)piperazine-1-carboxamide B7.

N-(2-Ethynylthiazol-4-yl)-4-(3′-(oxetan-3-ylamino)-[1,1′-biphenyl]-4-yl)piperazine-1-carboxamide B8. ¹H NMR (400 MHz, DMSO-d₆) δ 9.91 (s, 1H), 7.49-7.42 (m, 3H), 7.12 (t, J=7.8 Hz, 1H), 7.03 (d, J=8.8 Hz, 2H), 6.81 (d, J=7.9 Hz, 1H), 6.68-6.62 (m, 1H), 6.43-6.34 (m, 2H), 4.90 (s, 1H), 4.86 (t, J=6.4 Hz, 2H), 4.64-4.54 (m, 1H), 4.46-4.39 (m, 2H), 3.70-3.59 (m, 4H), 3.23-3.15 (m, 4H); MS (ESI) m/z: 460.2 [M+H⁺].

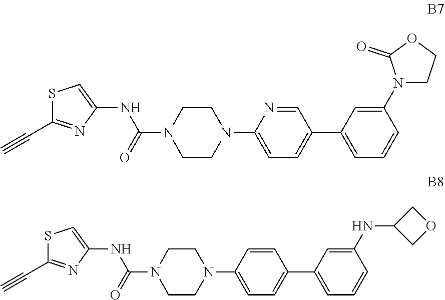 [SMILES: C#Cc1nc(NC(=O)N2CCN(c3ccc(-c4cccc(N5CCOC5=O)c4)cn3)CC2)cs1 ; C#Cc1nc(NC(=O)N2CCN(c3ccc(-c4cccc(NC5COC5)c4)cc3)CC2)cs1]

N-(2-Ethynylthiazol-4-yl)-4-(3′-((3-hydroxycyclobutyl)amino)-[1,1′-biphenyl]-4-yl)piperazine-1-carboxamide B9.

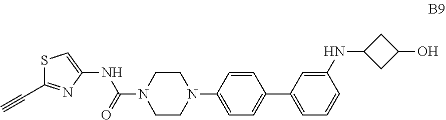 [SMILES: C#Cc1nc(NC(=O)N2CCN(c3ccc(-c4cccc(NC5CC(O)C5)c4)cc3)CC2)cs1]

N-(2-Ethynylthiazol-4-yl)-4-(3′-(3-hydroxyazetidin-1-yl)-[1,1′-biphenyl]-4-yl)piperazine-1-carboxamide B10.

N-(2-Ethynylthiazol-4-yl)-4-(4-(imidazo[1,5-a]pyridin-5-yl)phenyl)piperazine-1-carboxamide B11. ¹H NMR (400 MHz, DMSO-d₆) δ 9.93 (s, 1H), 7.81 (s, 1H), 7.57 (dd, J=16.8, 9.1 Hz, 4H), 7.46 (s, 1H), 7.31 (dd, J=9.0, 6.9 Hz, 1H), 7.16 (d, J=8.9 Hz, 2H), 6.82 (dt, J=5.9, 3.0 Hz, 1H), 4.91 (s, 1H), 3.78-3.54 (m, 4H), 3.31-3.25 (m, 4H); MS (ESI) m/z: 429.1 [M+H⁺].

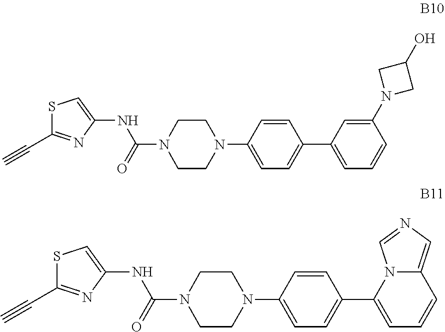 [SMILES: C#Cc1nc(NC(=O)N2CCN(c3ccc(-c4cccc(N5CC(O)C5)c4)cc3)CC2)cs1 ; C#Cc1nc(NC(=O)N2CCN(c3ccc(-c4cccc5cncn45)cc3)CC2)cs1]

4-(4-([1,2,4]Triazolo[4,3-a]pyridin-5-yl)phenyl)-N-(2-ethynylthiazol-4-yl)piperazine-1-carboxamide B12.

N-(2-Ethynylthiazol-4-yl)-4-(4-(imidazo[1,5-a]pyridin-8-yl)phenyl)piperazine-1-carboxamide B13.

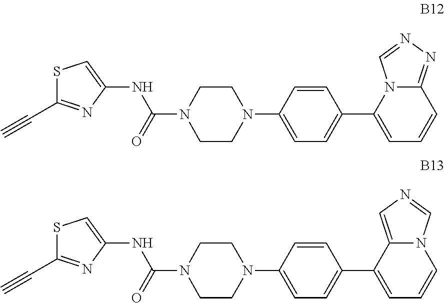 [SMILES: C#Cc1nc(NC(=O)N2CCN(c3ccc(-c4cccc5nncn45)cc3)CC2)cs1 ; C#Cc1nc(NC(=O)N2CCN(c3ccc(-c4cccn5cncc45)cc3)CC2)cs1]

4-(4-([1,2,3]Triazolo[1,5-a]pyridin-4-yl)phenyl)-N-(2-ethynylthiazol-4-yl)piperazine-1-carboxamide B14.

N-(2-Ethynylthiazol-4-yl)-4-(3′-(pyrrolidin-1-yl)-[1,1′-biphenyl]-4-yl)piperazine-1-carboxamide B15. ¹H NMR (400 MHz, DMSO-d₆) δ 9.91 (s, 1H), 7.51 (d, J=8.8 Hz, 2H), 7.45 (s, 1H), 7.18 (t, J=7.9 Hz, 1H), 7.04 (d, J=8.9 Hz, 2H), 6.80 (d, J=8.0 Hz, 1H), 6.70-6.65 (m, 1H), 6.46 (dd, J=8.1, 2.0 Hz, 1H), 4.90 (s, 1H), 3.66-3.61 (m, 4H), 3.28 (t, J=6.9 Hz, 5H), 3.22-3.15 (m, 4H), 1.96 (dd, J=8.0, 5.1 Hz, 4H); MS (ESI) m/z: 458.2 [M+H⁺].

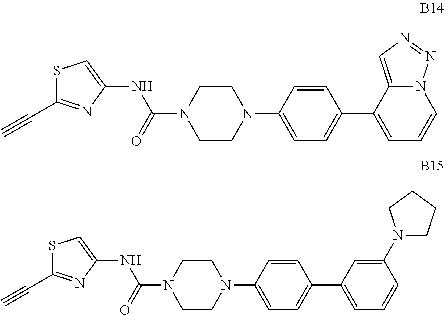 [SMILES: C#Cc1nc(NC(=O)N2CCN(c3ccc(-c4cccn5nncc45)cc3)CC2)cs1 ; C#Cc1nc(NC(=O)N2CCN(c3ccc(-c4cccc(N5CCCC5)c4)cc3)CC2)cs1]

(S)—N-(2-Ethynylthiazol-4-yl)-4-(3′-(3-hydroxypyrrolidin-1-yl)-[1,1′-biphenyl]-4-yl)piperazine-1-carboxamide B16.

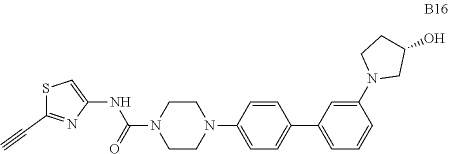 [SMILES: C#Cc1nc(NC(=O)N2CCN(c3ccc(-c4cccc(N5CC[C@H](O)C5)c4)cc3)CC2)cs1]

4-(4-(1,1-Dioxido-3-oxo-2,3-dihydrobenzo[b]thiophen-7-yl)phenyl)-N-(2-ethynylthiazol-4-yl)piperazine-1-carboxamide B17. ¹H NMR (400 MHz, DMSO-d₆) δ 9.93 (s, 1H), 7.95 (s, 3H), 7.63 (d, J=8.7 Hz, 2H), 7.46 (s, 1H), 7.11 (d, J=8.9 Hz, 2H), 4.90 (s, 1H), 4.62 (d, J=5.3 Hz, 2H), 3.70-3.59 (m, 4H), 3.31-3.27 (m, 4H); MS (ESI) m/z: 493.1 [M+H⁺].

N-(2-Ethynylthiazol-4-yl)-4-(4-(1-methyl-1H-indazol-4-yl)phenyl)piperazine-1-carboxamide B18. ¹H NMR (400 MHz, DMSO-d₆) δ 9.92 (s, 1H), 8.12 (s, 1H), 7.62 (d, J=8.7 Hz, 2H), 7.56 (d, J=8.4 Hz, 1H), 7.48-7.41 (m, 2H), 7.20 (d, J=6.9 Hz, 1H), 7.13 (d, J=8.8 Hz, 2H), 4.90 (s, 1H), 4.07 (s, 3H), 3.69-3.57 (m, 4H), 3.27-3.23 (m, 4H); MS (ESI) m/z: 443.2 [M+H⁺].

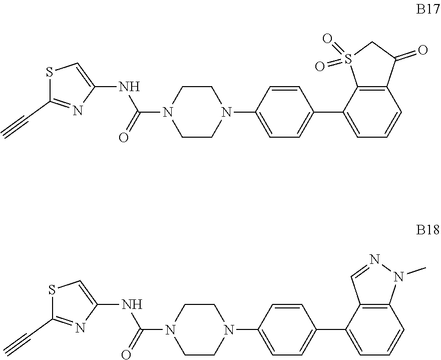 [SMILES: C#Cc1nc(NC(=O)N2CCN(c3ccc(-c4cccc5c4S(=O)(=O)CC5=O)cc3)CC2)cs1 ; C#Cc1nc(NC(=O)N2CCN(c3ccc(-c4cccc5c4cnn5C)cc3)CC2)cs1]

N-(2-Ethynylthiazol-4-yl)-4-(3′-(pyrrolidin-1-yl)-[1,1′-biphenyl]-4-yl)piperazine-1-carboxamide B19.

N-(2-Ethynylthiazol-4-yl)-4-(4-(1-oxo-1,2-dihydroisoquinolin-8-yl)phenyl)-piperazine-1-carboxamide B20. ¹H NMR (400 MHz, DMSO-d₆) δ 10.88 (d, J=5.5 Hz, 1H), 9.92 (s, 1H), 7.70-7.53 (m, 2H), 7.46 (s, 1H), 7.21-7.04 (m, 4H), 6.93 (d, J=8.7 Hz, 2H), 6.54 (dd, J=7.0, 1.2 Hz, 1H), 4.90 (s, 1H), 3.66 (dd, J=15.5, 10.9 Hz, 4H), 3.25-3.03 (m, 4H); MS (ESI) m/z: 456.1 [M+H⁺].

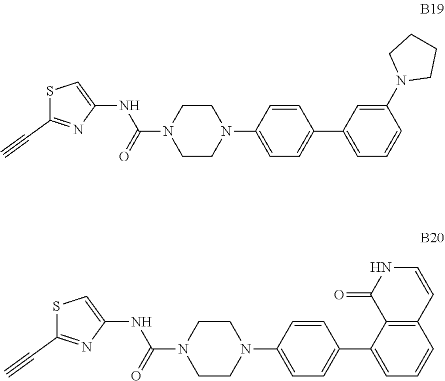 [SMILES: C#Cc1nc(NC(=O)N2CCN(c3ccc(-c4cccc(N5CCCC5)c4)cc3)CC2)cs1 ; C#Cc1nc(NC(=O)N2CCN(c3ccc(-c4cccc5cc[nH]c(=O)c45)cc3)CC2)cs1]

N-(2-Ethynylthiazol-4-yl)-4-(4-(3-hydroxy-1,1-dioxido-2,3-dihydrobenzo[b]-thiophen-7-yl)phenyl)piperazine-1-carboxamide B21. ¹H NMR (400 MHz, DMSO-d₆) δ 9.92 (s, 1H), 7.75 (t, J=7.6 Hz, 1H), 7.60 (d, J=7.6 Hz, 1H), 7.57-7.50 (m, 3H), 7.45 (s, 1H), 7.07 (d, J=8.9 Hz, 2H), 6.31 (d, J=6.0 Hz, 1H), 5.38 (q, J=5.8 Hz, 1H), 4.90 (s, 1H), 3.98 (q, J=13.2, 6.9 Hz, 1H), 3.71-3.54 (m, 4H), 3.29-3.23 (m, 4H); MS (ESI) m/z: 495.1 [M+H⁺].

N-(2-Ethynylthiazol-4-yl)-4-(4-(4-oxo-3,4-dihydroquinazolin-5-yl)phenyl)piperazine-1-carboxamide B22. ¹H NMR (400 MHz, DMSO-d₆) δ 11.93 (s, 1H), 9.92 (s, 1H), 8.04 (s, 1H), 7.74 (t, J=7.8 Hz, 1H), 7.61 (dd, J=8.1, 1.0 Hz, 1H), 7.46 (s, 1H), 7.22 (dd, J=7.4, 1.2 Hz, 1H), 7.20-7.14 (m, 2H), 6.95 (d, J=8.8 Hz, 2H), 4.90 (s, 1H), 3.68-3.56 (m, 4H), 3.23-3.13 (m, 4H); MS (ESI) m/z: 457.2 [M+H⁺].

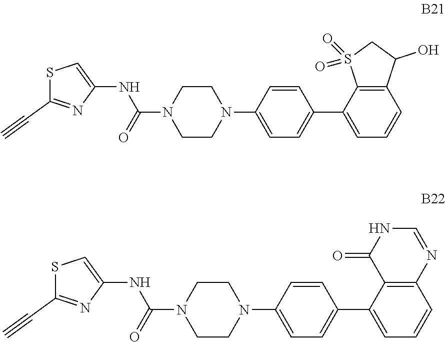 [SMILES: C#Cc1nc(NC(=O)N2CCN(c3ccc(-c4cccc5c4S(=O)(=O)CC5O)cc3)CC2)cs1 ; C#Cc1nc(NC(=O)N2CCN(c3ccc(-c4cccc5nc[nH]c(=O)c45)cc3)CC2)cs1]

(S)-4-(4-(Benzo[d]thiazol-7-yl)phenyl)-N-(2-ethynylthiazol-4-yl)-2-(hydroxy-methyl)piperazine-1-carboxamide B23. ¹H NMR (400 MHz, DMSO-d₆) δ 9.74 (s, 1H), 9.43 (s, 1H), 8.03 (dd, J=8.1, 1.0 Hz, 1H), 7.66-7.57 (m, 3H), 7.53 (d, J=6.8 Hz, 1H), 7.44 (s, 1H), 7.11 (d, J=8.9 Hz, 2H), 5.07 (t, J=5.2 Hz, 1H), 4.90 (s, 1H), 4.31 (s, 1H), 4.09 (d, J=13.4 Hz, 1H), 3.86 (d, J=12.0 Hz, 1H), 3.78-3.65 (m, 2H), 3.63-3.50 (m, 1H), 3.28-3.17 (m, 1H), 2.97 (dd, J=12.4, 3.9 Hz, 1H), 2.85 (td, J=11.6, 3.5 Hz, 1H); MS (ESI) m/z: 476.1 [M+H⁺].

(R)-4-(4-(Benzo[d]thiazol-7-yl)phenyl)-N-(2-ethynylthiazol-4-yl)-2-(hydroxy-methyl)piperazine-1-carboxamide B24. ¹H NMR (400 MHz, DMSO-d₆) δ 9.75 (s, 1H), 9.43 (s, 1H), 8.03 (dd, J=8.1, 1.0 Hz, 1H), 7.68-7.58 (m, 3H), 7.53 (d, J=6.9 Hz, 1H), 7.44 (s, 1H), 7.11 (d, J=8.9 Hz, 2H), 5.07 (t, J=5.2 Hz, 1H), 4.90 (s, 1H), 4.31 (s, 1H), 4.09 (d, J=13.2 Hz, 1H), 3.86 (d, J=12.0 Hz, 1H), 3.70 (dd, J=14.8, 9.0 Hz, 2H), 3.57 (dd, J=10.6, 5.1 Hz, 1H), 3.30-3.18 (m, 1H), 2.97 (dd, J=12.5, 3.9 Hz, 1H), 2.85 (td, J=11.5, 3.4 Hz, 1H); MS (ESI) m/z: 476.1 [M+H⁺].

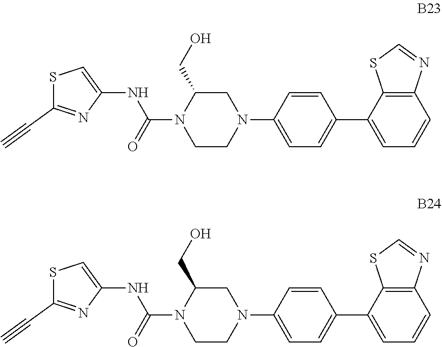 [SMILES: C#Cc1nc(NC(=O)N2CCN(c3ccc(-c4cccc5ncsc45)cc3)C[C@H]2CO)cs1 ; C#Cc1nc(NC(=O)N2CCN(c3ccc(-c4cccc5ncsc45)cc3)C[C@@H]2CO)cs1]

4-(4-(2-Amino-6-cyanobenzo[d]thiazol-7-yl)phenyl)-N-(2-ethynylthiazol-4-yl)-piperazine-1-carboxamide B25. ¹H NMR (400 MHz, DMSO-d₆) δ 9.93 (s, 1H), 8.01 (s, 2H), 7.69 (d, J=8.4 Hz, 1H), 7.46 (t, J=4.3 Hz, 3H), 7.36 (d, J=8.4 Hz, 1H), 7.13 (d, J=8.8 Hz, 2H), 4.90 (s, 1H), 3.65 (d, J=5.1 Hz, 4H), 3.31 (s, 4H); MS (ESI) m/z: 486.2 [M+H⁺].

4-(4-(6-Cyanobenzo[d]thiazol-7-yl)phenyl)-N-(2-ethynylthiazol-4-yl)piperazine-1-carboxamide B26. ¹H NMR (400 MHz, DMSO-d₆) δ 9.94 (s, 1H), 9.66 (s, 1H), 8.19 (d, J=8.5 Hz, 1H), 8.03 (d, J=8.5 Hz, 1H), 7.57 (d, J=8.8 Hz, 2H), 7.46 (d, J=3.7 Hz, 1H), 7.18 (d, J=8.9 Hz, 2H), 4.90 (s, 1H), 3.68-3.58 (m, 4H), 3.31-3.28 (m, 4H); MS (ESI) m/z: 471.1

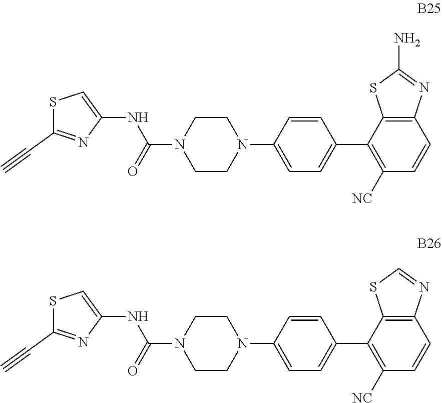 [SMILES: C#Cc1nc(NC(=O)N2CCN(c3ccc(-c4c(C#N)ccc5nc(N)sc45)cc3)CC2)cs1 ; C#Cc1nc(NC(=O)N2CCN(c3ccc(-c4c(C#N)ccc5ncsc45)cc3)CC2)cs1]

4-(4-(2-Aminobenzo[d]thiazol-7-yl)phenyl)-N-(2-ethynylthiazol-4-yl)piperazine-1-carboxamide B27. ¹H NMR (400 MHz, DMSO-d₆) δ 9.92 (s, 1H), 7.51 (d, J=8.8 Hz, 2H), 7.46 (s, 1H), 7.44 (s, 2H), 7.31-7.24 (m, 2H), 7.09 (d, J=8.8 Hz, 2H), 7.05 (dd, J=5.3, 3.4 Hz, 1H), 4.90 (s, 1H), 3.74-3.50 (m, 4H), 3.26-3.18 (m, 4H); MS (ESI) m/z: 461.1 [M+H⁺].

4-(4-(Benzo[d][1,2,3]thiadiazol-7-yl)phenyl)-N-(2-ethynylthiazol-4-yl)piperazine-1-carboxamide B28. ¹H NMR (400 MHz, DMSO-d₆) δ 9.93 (s, 1H), 8.66 (d, J=8.2 Hz, 1H), 7.94 (d, J=6.8 Hz, 1H), 7.87 (d, J=8.0 Hz, 1H), 7.65 (d, J=8.8 Hz, 2H), 7.46 (s, 1H), 7.18 (d, J=8.8 Hz, 2H), 4.91 (s, 1H), 3.83-3.56 (m, 4H), 3.29 (d, J=4.9 Hz, 4H); MS (ESI) m/z: 447.1 [M+H⁺].

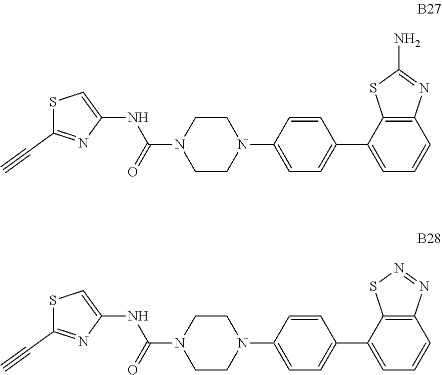 [SMILES: C#Cc1nc(NC(=O)N2CCN(c3ccc(-c4cccc5nc(N)sc45)cc3)CC2)cs1 ; C#Cc1nc(NC(=O)N2CCN(c3ccc(-c4cccc5nnsc45)cc3)CC2)cs1]

4-(4-([1,2,4]Triazolo[1,5-a]pyridin-8-yl)phenyl)-N-(2-ethynylthiazol-4-yl)-piperazine-1-carboxamide B29. ¹H NMR (400 MHz, DMSO-d₆) δ 9.93 (s, 1H), 8.86 (dd, J=6.7, 1.0 Hz, 1H), 8.54 (s, 1H), 8.13 (d, J=8.9 Hz, 2H), 7.87 (dd, J=7.4, 1.0 Hz, 1H), 7.46 (s, 1H), 7.26 (t, J=7.1 Hz, 1H), 7.12 (d, J=9.0 Hz, 2H), 4.91 (s, 1H), 3.69-3.61 (m, 4H), 3.30-3.25 (m, 4H); MS (ESI) m/z: 430.1 [M+H⁺].

4-(4-([1,2,4]Triazolo[4,3-a]pyridin-8-yl)phenyl)-N-(2-ethynylthiazol-4-yl)-piperazine-1-carboxamide B30. ¹H NMR (400 MHz, DMSO-d₆) δ 9.92 (s, 1H), 9.32 (s, 1H), 8.49 (d, J=6.7 Hz, 1H), 8.18 (d, J=8.9 Hz, 2H), 7.58 (d, J=7.0 Hz, 1H), 7.39 (d, J=21.1 Hz, 1H), 7.12 (d, J=9.0 Hz, 2H), 7.04 (t, J=6.9 Hz, 1H), 4.85 (s, 1H), 3.68-3.61 (m, 4H), 3.28 (d, J=4.8 Hz, 4H); MS (ESI) m/z: 430.1 [M+H⁺].

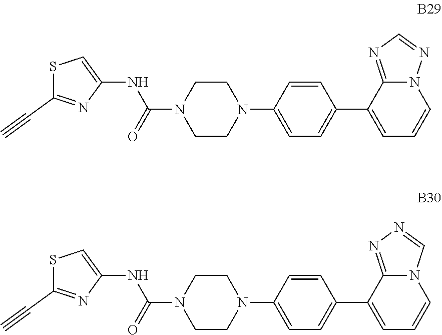 [SMILES: C#Cc1nc(NC(=O)N2CCN(c3ccc(-c4cccn5ncnc45)cc3)CC2)cs1 ; C#Cc1nc(NC(=O)N2CCN(c3ccc(-c4cccn5cnnc45)cc3)CC2)cs1]

4-(2′-Cyano-[1,1′-biphenyl]-4-yl)-N-(2-ethynylthiazol-4-yl)piperazine-1-carboxamide B31. ¹H NMR (400 MHz, DMSO-d₆) δ 9.92 (s, 1H), 7.89 (dd, J=7.8, 1.2 Hz, 1H), 7.79-7.68 (m, 1H), 7.62-7.56 (m, 1H), 7.56-7.45 (m, 4H), 7.15-7.08 (m, 2H), 4.90 (s, 1H), 3.71-3.58 (m, 4H), 3.30-3.25 (m, 4H); MS (ESI) m/z: 414.1 [M+H⁺].

4-(4-([1,2,4]Triazolo[4,3-a]pyridin-5-yl)phenyl)-N-(2-ethynylthiazol-4-yl)-piperazine-1-carboxamide B32. ¹H NMR (400 MHz, DMSO-d₆) δ 9.93 (s, 1H), 9.19 (s, 1H), 7.73 (d, J=9.2 Hz, 1H), 7.65 (d, J=8.6 Hz, 2H), 7.49-7.40 (m, 2H), 7.16 (d, J=8.7 Hz, 2H), 6.92 (d, J=6.8 Hz, 1H), 4.90 (s, 1H), 3.66 (s, 4H), 3.33 (s, 4H); MS (ESI) m/z: 430.1 [M+H⁺].

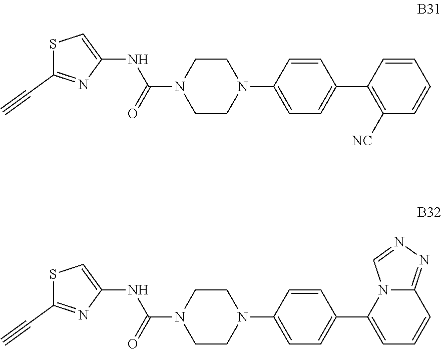 [SMILES: C#Cc1nc(NC(=O)N2CCN(c3ccc(-c4ccccc4C#N)cc3)CC2)cs1 ; C#Cc1nc(NC(=O)N2CCN(c3ccc(-c4cccc5nncn45)cc3)CC2)cs1]

N-(2-Ethynylthiazol-4-yl)-4-(4-(imidazo[1,2-a]pyridin-5-yl)phenyl)piperazine-1-carboxamide B33.

4-(4-([1,2,4]Triazolo[1,5-a]pyridin-5-yl)phenyl)-N-(2-ethynylthiazol-4-yl)-piperazine-1-carboxamide B34. ¹H NMR (400 MHz, DMSO-d₆) δ 9.94 (s, 1H), 8.52 (s, 1H), 7.98 (d, J=9.0 Hz, 2H), 7.77 (dd, J=8.8, 1.4 Hz, 1H), 7.72 (dd, J=8.8, 7.0 Hz, 1H), 7.46 (s, 1H), 7.33 (dd, J=7.0, 1.4 Hz, 1H), 7.14 (d, J=9.1 Hz, 2H), 4.91 (s, 1H), 3.67-3.64 (m, 4H), 3.34-3.32 (m, 4H); MS (ESI) m/z: 430.1 [M+H⁺].

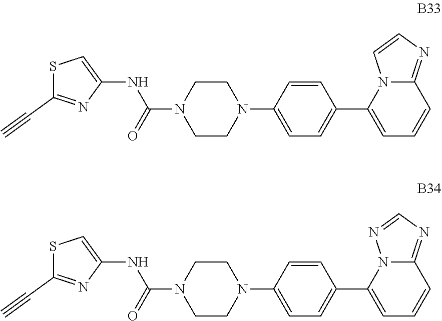 [SMILES: C#Cc1nc(NC(=O)N2CCN(c3ccc(-c4cccc5nccn45)cc3)CC2)cs1 ; C#Cc1nc(NC(=O)N2CCN(c3ccc(-c4cccc5ncnn45)cc3)CC2)cs1]

N-(2-Ethynylthiazol-4-yl)-4-(4-(imidazo[1,2-a]pyridin-8-yl)phenyl)piperazine-1-carboxamide B35. ¹H NMR (400 MHz, DMSO-d₆) δ 9.93 (s, 1H), 8.57 (d, J=6.6 Hz, 1H), 8.11 (s, 1H), 7.99 (d, J=8.6 Hz, 2H), 7.74 (s, 1H), 7.52 (d, J=7.1 Hz, 1H), 7.46 (s, 1H), 7.11 (d, J=8.9 Hz, 3H), 4.90 (s, 1H), 3.68-3.63 (m, 4H), 3.28-3.25 (m, 4H); MS (ESI) m/z: 429.1 [M+H⁺].

(R)-4-(4-(Benzo[d]thiazol-7-yl)phenyl)-N-(2-ethynylthiazol-4-yl)-3-(hydroxy-methyl)piperazine-1-carboxamide B36. ¹H NMR (400 MHz, DMSO-d₆) δ 9.80 (s, 1H), 9.44 (s, 1H), 8.03 (dd, J=0.8 Hz, 8.0 Hz, 1H), 7.52-7.67 (m, 4H), 7.47 (s, 1H), 7.07 (d, J=8.8 Hz, 2H), 4.87-4.94 (m, 2H), 4.24-4.31 (m, 1H), 4.02-4.11 (m, 1H), 3.86-3.94 (m, 1H), 3.38-3.62 (m, 3H), 3.18-3.31 (m, 3H); MS (ESI) m/z: 476.1 [M+H⁺].

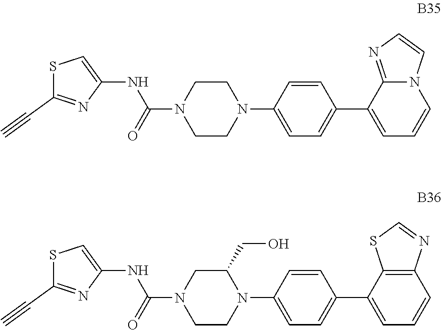 [SMILES: C#Cc1nc(NC(=O)N2CCN(c3ccc(-c4cccn5ccnc45)cc3)CC2)cs1 ; C#Cc1nc(NC(=O)N2CCN(c3ccc(-c4cccc5ncsc45)cc3)[C@@H](CO)C2)cs1]

(S)-4-(4-(Benzo[d]thiazol-7-yl)phenyl)-N-(2-ethynylthiazol-4-yl)-3-(hydroxy-methyl)piperazine-1-carboxamide B37. ¹H NMR (400 MHz, DMSO-d₆) δ 9.79 (s, 1H), 9.43 (s, 1H), 8.02 (dd, J=8.0, 1.0 Hz, 1H), 7.65-7.57 (m, 3H), 7.53 (d, J=6.9 Hz, 1H), 7.46 (s, 1H), 7.07 (d, J=8.9 Hz, 2H), 4.95-4.86 (m, 2H), 4.26 (d, J=12.9 Hz, 1H), 4.06 (d, J=13.0 Hz, 1H), 3.89 (d, J=4.4 Hz, 1H), 3.57 (dd, J=7.3, 4.9 Hz, 1H), 3.47 (dd, J=9.3, 6.3 Hz, 1H), 3.41 (dd, J=10.0, 5.0 Hz, 1H), 3.28-3.23 (m, 1H), 3.23-3.19 (m, 1H), 3.16 (d, J=9.7 Hz, 1H); MS (ESI) m/z: 476.1 [M+H⁺].

4-(3′-(1-Cyanocyclopropyl)-[1,1′-biphenyl]-4-yl)-N-(2-ethynylthiazol-4-yl)-piperazine-1-carboxamide B38. ¹H NMR (400 MHz, DMSO-d₆) δ 9.92 (s, 1H), 7.58 (s, 1H), 7.57-7.52 (m, 2H), 7.46 (t, J=1.7 Hz, 1H), 7.46-7.40 (m, 2H), 7.29-7.24 (m, 1H), 7.07 (d, J=8.9 Hz, 2H), 4.90 (s, 1H), 3.69-3.58 (m, 4H), 3.26-3.15 (m, 4H), 1.76 (d, J=3.0 Hz, 2H), 1.60 (d, J=2.8 Hz, 2H); MS (ESI) m/z: 454.2 [M+H⁺].

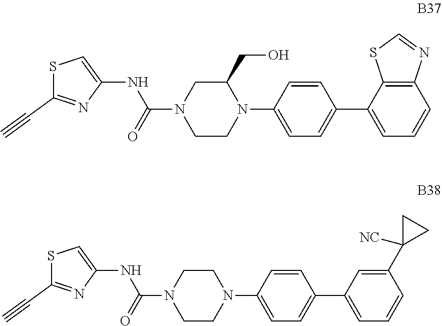 [SMILES: C#Cc1nc(NC(=O)N2CCN(c3ccc(-c4cccc5ncsc45)cc3)[C@H](CO)C2)cs1 ; C#Cc1nc(NC(=O)N2CCN(c3ccc(-c4cccc(C5(C#N)CC5)c4)cc3)CC2)cs1]

4-(3′-Cyano-[1,1′-biphenyl]-4-yl)-N-(2-ethynylthiazol-4-yl)piperazine-1-carboxamide B39. ¹H NMR (400 MHz, DMSO-d₆) δ 9.92 (s, 1H), 8.16 (s, 1H), 8.09 (t, J=1.5 Hz, 1H), 8.01-7.92 (m, 1H), 7.72 (dt, J=7.6, 1.2 Hz, 1H), 7.68-7.58 (m, 3H), 7.45 (s, 1H), 7.08 (d, J=8.9 Hz, 2H), 4.90 (s, 1H), 3.68-3.60 (m, 4H), 3.26-3.22 (m, 4H); MS (ESI) m/z: 414.1 [M+H⁺].

4-(4-(Benzo[d]thiazol-7-yl)-2-cyanophenyl)-N-(2-ethynylthiazol-4-yl)piperazine-1-carboxamide B40. ¹H NMR (400 MHz, DMSO-d₆) δ 9.94 (s, 1H), 9.47 (s, 1H), 8.12 (dd, J=7.9, 1.1 Hz, 1H), 8.06 (d, J=2.3 Hz, 1H), 7.97 (dd, J=8.6, 2.3 Hz, 1H), 7.67 (t, J=7.7 Hz, 1H), 7.62 (d, J=6.5 Hz, 1H), 7.47 (s, 1H), 7.37 (d, J=8.7 Hz, 1H), 4.90 (s, 1H), 3.75-3.67 (m, 4H), 3.30-3.24 (m, 4H); MS (ESI) m/z: 471.1 [M+H⁺].

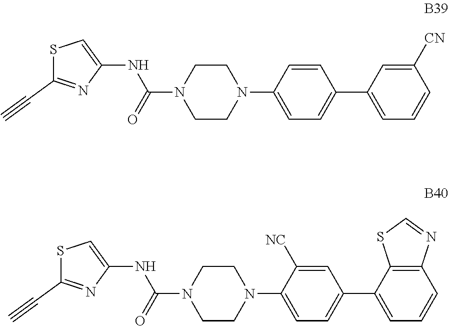 [SMILES: C#Cc1nc(NC(=O)N2CCN(c3ccc(-c4cccc(C#N)c4)cc3)CC2)cs1 ; C#Cc1nc(NC(=O)N2CCN(c3ccc(-c4cccc5ncsc45)cc3C#N)CC2)cs1]

4-(4-(Benzo[d]thiazol-7-yl)-3-cyanophenyl)-N-(2-ethynylthiazol-4-yl)piperazine-1-carboxamide B41. ¹H NMR (400 MHz, DMSO-d₆) δ 9.95 (s, 1H), 9.45 (s, 1H), 8.16 (dd, J=8.2, 1.0 Hz, 1H), 7.71-7.65 (m, 1H), 7.61 (d, J=8.8 Hz, 1H), 7.55 (dd, J=9.4, 4.8 Hz, 2H), 7.48-7.40 (m, 2H), 4.91 (s, 1H), 3.68-3.62 (m, 4H), 3.40-3.36 (m, 4H); MS (ESI) m/z: 471.1 [M+H⁺].

4-(3′-Cyclopropyl-[1,1′-biphenyl]-4-yl)-N-(2-ethynylthiazol-4-yl)piperazine-1-carboxamide B42. ¹H NMR (400 MHz, DMSO-d₆) δ 9.91 (s, 1H), 7.53 (d, J=8.8 Hz, 2H), 7.45 (s, 1H), 7.37-7.33 (m, 1H), 7.30-7.24 (m, 2H), 7.05 (d, J=8.9 Hz, 2H), 6.99-6.94 (m, 1H), 4.90 (s, 1H), 3.64 (t, 5H), 3.20 (t, 5H), 2.01-1.91 (m, 1H), 1.00-0.91 (m, 2H), 0.77-0.68 (m, 2H); MS (ESI) m/z: 429.2 [M+H⁺].

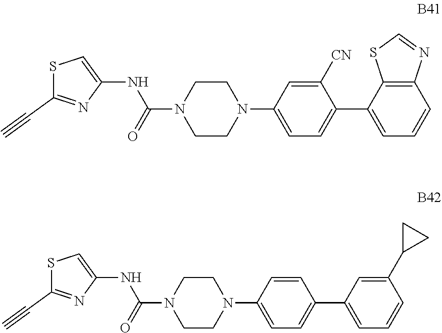 [SMILES: C#Cc1nc(NC(=O)N2CCN(c3ccc(-c4cccc5ncsc45)c(C#N)c3)CC2)cs1 ; C#Cc1nc(NC(=O)N2CCN(c3ccc(-c4cccc(C5CC5)c4)cc3)CC2)cs1]

N-(2-Ethynylthiazol-4-yl)-4-(3′-(2-oxopyrrolidin-1-yl)-[1,1′-biphenyl]-4-yl)-piperazine-1-carboxamide B43. ¹H NMR (400 MHz, DMSO-d₆) δ 9.92 (s, 1H), 7.87 (s, 1H), 7.57-7.52 (m, 3H), 7.45 (s, 1H), 7.40 (t, J=7.8 Hz, 1H), 7.38-7.34 (m, 1H), 7.07 (d, J=8.9 Hz, 2H), 4.90 (s, 1H), 3.90 (t, J=7.0 Hz, 2H), 3.68-3.60 (m, 4H), 3.24-3.16 (m, 4H), 2.53 (d, J=3.8 Hz, 2H), 2.13-2.03 (m, 2H); MS (ESI) m/z: 472.2 [M+H⁺].

4-((4-(Benzo[d]thiazol-7-yl)phenyl)amino)-N-(2-ethynylthiazol-4-yl)piperidine-1-carboxamide B44. ¹H NMR (400 MHz, DMSO-d₆) δ 9.80 (s, 1H), 9.41 (s, 1H), 7.98 (dd, J=8.1, 0.8 Hz, 1H), 7.59 (t, J=7.8 Hz, 1H), 7.47 (dd, J=8.1, 2.6 Hz, 3H), 7.43 (s, 1H), 6.76 (d, J=8.7 Hz, 2H), 5.91 (d, J=8.0 Hz, 1H), 4.89 (s, 1H), 4.10 (d, J=13.6 Hz, 2H), 3.62-3.47 (m, 1H), 3.03 (t, J=11.4 Hz, 2H), 1.95 (d, J=10.3 Hz, 2H), 1.33 (dd, J=20.7, 9.9 Hz, 2H).

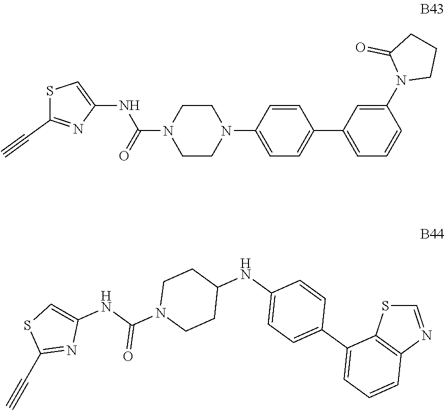 [SMILES: C#Cc1nc(NC(=O)N2CCN(c3ccc(-c4cccc(N5CCCC5=O)c4)cc3)CC2)cs1 ; C#Cc1nc(NC(=O)N2CCC(Nc3ccc(-c4cccc5ncsc45)cc3)CC2)cs1]

4-(4-(2-Amino-[1,2,4]triazolo[1,5-a]pyridin-5-yl)phenyl)-N-(2-ethynylthiazol-4-yl)piperazine-1-carboxamide B45. ¹H NMR (400 MHz, DMSO-d₆) δ 9.93 (s, 1H), 7.92 (d, J=9.0 Hz, 2H), 7.46 (q, J=7.1 Hz, 2H), 7.27 (dd, J=8.7, 1.2 Hz, 1H), 7.10 (d, J=9.1 Hz, 2H), 6.98 (dd, J=7.4, 1.2 Hz, 1H), 5.99 (s, 2H), 4.91 (s, 1H), 3.67-3.64 (m, 4H), 3.32-3.28 (m, 4H); MS (ESI) m/z: 445.2 [M+H⁺].

4-(3′-(Cyclopent-1-en-1-yl)-[1,1′-biphenyl]-4-yl)-N-(2-ethynylthiazol-4-yl)-piperazine-1-carboxamide B46. ¹H NMR (400 MHz, DMSO-d₆) δ 9.92 (s, 1H), 7.63 (s, 1H), 7.57 (d, J=8.8 Hz, 2H), 7.46 (d, J=6.2 Hz, 2H), 7.37 (d, J=5.4 Hz, 2H), 7.06 (d, J=8.8 Hz, 2H), 6.36 (s, 1H), 4.90 (s, 1H), 3.68-3.59 (m, 4H), 3.25-3.14 (m, 4H), 2.71 (dd, J=10.4, 4.5 Hz, 2H), 2.04-1.92 (m, 2H); MS (ESI) m/z: 455.2 [M+H⁺].

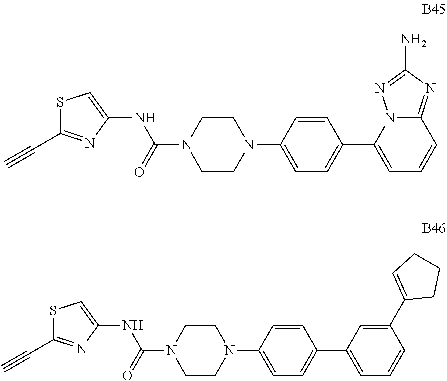 [SMILES: C#Cc1nc(NC(=O)N2CCN(c3ccc(-c4cccc5nc(N)nn45)cc3)CC2)cs1 ; C#Cc1nc(NC(=O)N2CCN(c3ccc(-c4cccc(C5=CCCC5)c4)cc3)CC2)cs1]

N-(2-Ethynylthiazol-4-yl)-4-(3′-(2-oxoimidazolidin-1-yl)-[1,1′-biphenyl]-4-yl)-piperazine-1-carboxamide B47. ¹H NMR (400 MHz, DMSO-d₆) δ 9.92 (s, 1H), 7.78 (s, 1H), 7.52 (d, J=8.7 Hz, 2H), 7.46 (d, J=6.3 Hz, 2H), 7.33 (t, J=7.9 Hz, 1H), 7.21 (d, J=7.8 Hz, 1H), 7.06 (d, J=8.8 Hz, 2H), 6.96 (s, 1H), 4.90 (s, 1H), 3.95-3.87 (m, 2H), 3.69-3.59 (m, 4H), 3.45-3.39 (m, 2H), 3.24-3.15 (m, 4H); MS (ESI) m/z: 473.1 [M+H⁺].

N-(2-Ethynylthiazol-4-yl)-4-(3′-(1-hydroxycyclopentyl)-[1,1′-biphenyl]-4-yl)-piperazine-1-carboxamide B48. ¹H NMR (400 MHz, DMSO-d₆) δ 9.91 (s, 1H), 7.69 (s, 1H), 7.54 (d, J=8.8 Hz, 2H), 7.45 (s, 1H), 7.41 (dt, J=6.9, 1.8 Hz, 1H), 7.38-7.31 (m, 2H), 7.06 (d, J=8.9 Hz, 2H), 4.90 (s, 1H), 4.80 (s, 1H), 3.72-3.58 (m, 4H), 3.25-3.14 (m, 4H), 1.89 (s, 6H), 1.75 (s, 2H); MS (ESI) m/z: 473.2 [M+H⁺].

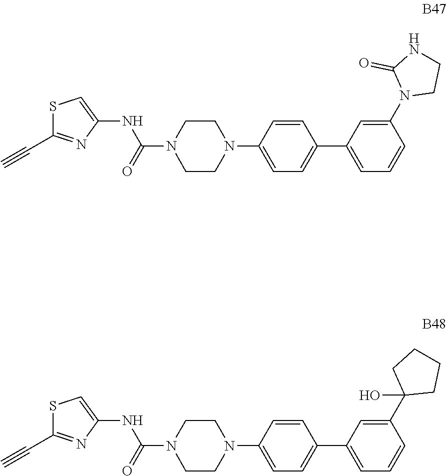 [SMILES: C#Cc1nc(NC(=O)N2CCN(c3ccc(-c4cccc(N5CCNC5=O)c4)cc3)CC2)cs1 ; C#Cc1nc(NC(=O)N2CCN(c3ccc(-c4cccc(C5(O)CCCC5)c4)cc3)CC2)cs1]

N-(2-Ethynylthiazol-4-yl)-4-(3′-(3-hydroxyazetidin-1-yl)-[1,1′-biphenyl]-4-yl)-piperazine-1-carboxamide B49. ¹H NMR (400 MHz, DMSO-d₆) δ 9.91 (s, 1H), 7.50 (d, J=8.8 Hz, 2H), 7.45 (s, 1H), 7.19 (t, J=7.8 Hz, 1H), 7.03 (d, J=8.9 Hz, 2H), 6.89 (d, J=7.9 Hz, 1H), 6.58 (s, 1H), 6.35 (dd, J=8.0, 1.6 Hz, 1H), 5.58 (d, J=6.6 Hz, 1H), 4.90 (s, 1H), 4.57 (d, J=6.2 Hz, 1H), 4.10 (t, J=7.1 Hz, 2H), 3.68-3.61 (m, 4H), 3.53 (dd, J=7.8, 5.1 Hz, 2H), 3.27-3.07 (m, 4H); MS (ESI) m/z: 460.1 [M+H⁺].

N-(2-Ethynylthiazol-4-yl)-4-(3′-(3-hydroxypyrrolidin-1-yl)-[1,1′-biphenyl]-4-yl)-piperazine-1-carboxamide B50. ¹H NMR (400 MHz, DMSO-d₆) δ 9.91 (s, 1H), 7.51 (d, J=8.8 Hz, 2H), 7.45 (s, 1H), 7.18 (t, J=7.9 Hz, 1H), 7.04 (d, J=8.8 Hz, 2H), 6.80 (d, J=7.9 Hz, 1H), 6.65 (s, 1H), 6.44 (dd, J=8.2, 1.9 Hz, 1H), 4.94 (d, J=3.8 Hz, 1H), 4.90 (s, 1H), 4.41 (s, 1H), 3.75-3.55 (m, 4H), 3.45 (s, 1H), 3.35 (d, J=9.4 Hz, 2H), 3.25-3.16 (m, 4H), 3.16-3.07 (m, 1H), 2.15-1.98 (m, 1H), 1.96-1.78 (m, 1H); MS (ESI) m/z: 474.2 [M+H⁺].

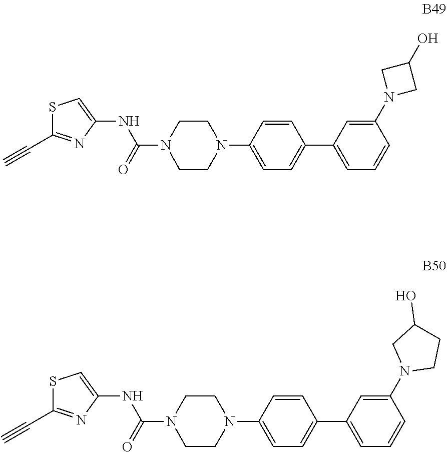 [SMILES: C#Cc1nc(NC(=O)N2CCN(c3ccc(-c4cccc(N5CC(O)C5)c4)cc3)CC2)cs1 ; C#Cc1nc(NC(=O)N2CCN(c3ccc(-c4cccc(N5CCC(O)C5)c4)cc3)CC2)cs1]

4-(3′-(Azetidin-1-yl)-[1,1′-biphenyl]-4-yl)-N-(2-ethynylthiazol-4-yl)piperazine-1-carboxamide B51. ¹H NMR (400 MHz, DMSO-d₆) δ 9.91 (s, 1H), 7.49 (d, J=8.8 Hz, 2H), 7.45 (s, 1H), 7.19 (t, J=7.8 Hz, 1H), 7.03 (d, J=8.8 Hz, 2H), 6.88 (d, J=8.2 Hz, 1H), 6.56 (t, J=1.8 Hz, 1H), 6.33 (dd, J=8.0, 1.6 Hz, 1H), 4.90 (p, 1H), 3.83 (t, J=7.2 Hz, 4H), 3.63 (t, 4H), 3.19 (t, 5H), 2.31 (p, 2H); MS (ESI) nm/z: 444.2 [M+H⁺].

(S)—N-(2-Ethynylthiazol-4-yl)-2-(hydroxymethyl)-4-(3′-(pyrrolidin-1-yl)-[1,1′-biphenyl]-4-yl)piperazine-1-carboxamide B52. ¹H NMR (400 MHz, DMSO-d₆) δ 9.71 (s, 1H), 7.50 (d, J=8.8 Hz, 2H), 7.43 (s, 1H), 7.18 (t, J=7.9 Hz, 1H), 7.00 (d, J=8.8 Hz, 2H), 6.79 (d, J=8.0 Hz, 1H), 6.67 (s, 1H), 6.46 (dd, J=8.1, 1.9 Hz, 1H), 5.03 (t, J=5.2 Hz, 1H), 4.90 (s, 1H), 4.29 (s, 1H), 4.07 (d, J=13.4 Hz, 1H), 3.79 (d, J=12.1 Hz, 1H), 3.68 (ddd, J=24.5, 13.6, 9.7 Hz, 2H), 3.60-3.51 (m, 1H), 3.27 (t, J=6.5 Hz, 5H), 3.24-3.13 (m, 1H), 2.87 (dd, J=12.4, 3.9 Hz, 1H), 2.75 (td, J=11.6, 3.4 Hz, 1H), 1.97 (t, J=6.5 Hz, 4H); MS (ESI) m/z: 488.1 [M+H⁺].

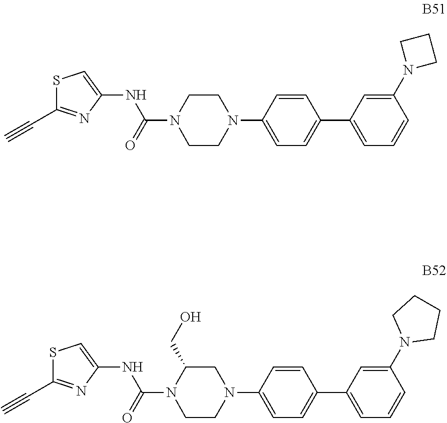 [SMILES: C#Cc1nc(NC(=O)N2CCN(c3ccc(-c4cccc(N5CCC5)c4)cc3)CC2)cs1 ; C#Cc1nc(NC(=O)N2CCN(c3ccc(-c4cccc(N5CCCC5)c4)cc3)C[C@H]2CO)cs1]

(R)-4-(3-Cyano-3′-(pyrrolidin-1-yl)-[1,1′-biphenyl]-4-yl)-N-(2-ethynylthiazol-4-yl)-2-(hydroxymethyl)piperazine-1-carboxamide B53. ¹H NMR (400 MHz, DMSO-d₆) δ 9.67 (s, 1H), 8.00 (d, J=2.2 Hz, 1H), 7.88 (dd, J=8.6, 2.2 Hz, 1H), 7.43 (s, 1H), 7.26 (d, J=8.8 Hz, 1H), 7.25-7.20 (m, 1H), 6.86 (d, J=7.7 Hz, 1H), 6.74 (s, 1H), 6.56-6.50 (m, 1H), 4.95 (t, J=5.0 Hz, 1H), 4.90 (s, 1H), 4.36 (d, J=7.5 Hz, 1H), 4.14 (d, J=13.0 Hz, 1H), 3.80-3.72 (m, 2H), 3.54 (d, J=11.2 Hz, 2H), 3.28 (d, J=6.2 Hz, 4H), 3.23 (s, 1H), 3.04 (dd, J=12.1, 3.5 Hz, 1H), 2.88 (dd, J=11.7, 9.0 Hz, 1H), 1.97 (t, J=6.4 Hz, 4H); MS (ESI) m/z: 513.2 [M+H⁺].

(R)—N-(2-Ethynylthiazol-4-yl)-2-(hydroxymethyl)-4-(3′-(pyrrolidin-1-yl)-[1,1′-biphenyl]-4-yl)piperazine-1-carboxamide B54. ¹H NMR (400 MHz, DMSO-d₆) δ 9.72 (s, 1H), 7.50 (d, J=8.8 Hz, 2H), 7.43 (s, 1H), 7.18 (t, J=7.9 Hz, 1H), 7.00 (d, J=8.9 Hz, 2H), 6.79 (d, J=8.0 Hz, 1H), 6.67 (d, J=1.9 Hz, 1H), 6.46 (dd, J=8.2, 1.9 Hz, 1H), 5.03 (t, J=5.2 Hz, 1H), 4.90 (s, 1H), 4.29 (s, 1H), 4.07 (d, J=13.3 Hz, 1H), 3.79 (d, J=12.1 Hz, 1H), 3.70 (ddd, J=10.5, 7.7, 5.9 Hz, 1H), 3.64 (d, J=11.8 Hz, 1H), 3.59-3.51 (m, 1H), 3.27 (t, J=6.5 Hz, 4H), 3.20 (dd, J=17.3, 7.4 Hz, 1H), 2.87 (dd, J=12.3, 3.9 Hz, 1H), 2.75 (td, J=11.6, 3.5 Hz, 1H), 1.99-1.93 (m, 4H); MS (ESI) m/z: 488.2 [M+H⁺].

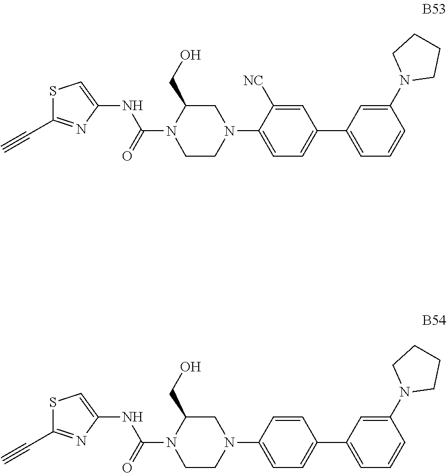 [SMILES: C#Cc1nc(NC(=O)N2CCN(c3ccc(-c4cccc(N5CCCC5)c4)cc3C#N)C[C@@H]2CO)cs1 ; C#Cc1nc(NC(=O)N2CCN(c3ccc(-c4cccc(N5CCCC5)c4)cc3)C[C@@H]2CO)cs1]

N-(2-Ethynylthiazol-4-yl)-4-(3′-(1-hydroxycyclobutyl)-[1,1′-biphenyl]-4-yl)-piperazine-1-carboxamide B55. ¹H NMR (400 MHz, DMSO-d₆) δ 9.92 (s, 1H), 7.67 (s, 1H), 7.54 (d, J=8.7 Hz, 2H), 7.49-7.43 (m, 2H), 7.41-7.33 (m, 2H), 7.07 (d, J=8.8 Hz, 2H), 5.50 (s, 1H), 4.90 (s, 1H), 3.67-3.58 (m, 4H), 3.23-3.17 (m, 4H), 2.47-2.36 (m, 2H), 2.33-2.24 (m, 2H), 2.00-1.89 (m, 1H), 1.73-1.61 (m, 1H); MS (ESI) m/z: 459.2 [M+H⁺].

(R)—N-(2-Ethynylthiazol-4-yl)-2-(hydroxymethyl)-4-(5-(3-(pyrrolidin-1-yl)-phenyl)pyridin-2-yl)piperazine-1-carboxamide B56. ¹H NMR (400 MHz, DMSO-d₆) δ 9.73 (s, 1H), 8.41 (d, J=2.3 Hz, 1H), 7.82 (dd, J=8.8, 2.5 Hz, 1H), 7.42 (s, 1H), 7.20 (t, J=7.9 Hz, 1H), 6.88 (d, J=8.9 Hz, 1H), 6.80 (d, J=8.1 Hz, 1H), 6.70-6.65 (m, 1H), 6.48 (dd, J=8.2, 1.9 Hz, 1H), 5.08 (t, J=4.9 Hz, 1H), 4.90 (s, 1H), 4.27 (dd, J=14.6, 7.8 Hz, 2H), 4.19-4.01 (m, 2H), 3.60-3.45 (m, 2H), 3.28 (t, J=6.6 Hz, 4H), 3.25-3.16 (m, 2H), 3.00 (dd, J=11.5, 3.2 Hz, 1H), 2.02-1.90 (m, 4H); MS (ESI) m/z: 489.3 [M+H⁺].

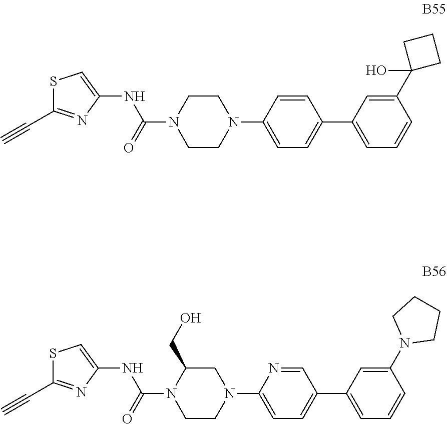 [SMILES: C#Cc1nc(NC(=O)N2CCN(c3ccc(-c4cccc(C5(O)CCC5)c4)cc3)CC2)cs1 ; C#Cc1nc(NC(=O)N2CCN(c3ccc(-c4cccc(N5CCCC5)c4)cn3)C[C@@H]2CO)cs1]

N-(2-Ethynylthiazol-4-yl)-4-(5-(3-(2-oxooxazolidin-3-yl)phenyl)pyridin-2-yl)-piperazine-1-carboxamide B57. ¹H NMR (400 MHz, DMSO-d₆) δ 9.92 (s, 1H), 7.77 (t, J=1.8 Hz, 1H), 7.55 (d, J=8.8 Hz, 2H), 7.50-7.46 (m, 1H), 7.45 (s, 1H), 7.41 (d, J=8.0 Hz, 1H), 7.36 (dd, J=6.2, 1.4 Hz, 1H), 7.08 (d, J=8.9 Hz, 2H), 4.90 (s, 1H), 4.46 (dd, J=8.9, 7.0 Hz, 2H), 4.14 (dd, J=8.9, 7.0 Hz, 2H), 3.66-3.62 (m, 4H), 3.23-3.19 (m, 4H); MS (ESI) m/z: 474.2 [M+H⁺].

(R)-4-(5-(Benzo[d]thiazol-7-yl)pyridin-2-yl)-N-(2-ethynylthiazol-4-yl)-2-(hydroxymethyl)piperazine-1-carboxamide B58. ¹H NMR (400 MHz, DMSO-d₆) δ 9.76 (s, 1H), 9.45 (s, 1H), 8.49 (d, J=2.3 Hz, 1H), 8.06 (dd, J=8.1, 1.0 Hz, 1H), 7.92 (dd, J=8.9, 2.6 Hz, 1H), 7.64 (t, J=7.8 Hz, 1H), 7.56 (d, J=6.8 Hz, 1H), 7.44 (s, 1H), 6.99 (d, J=8.9 Hz, 1H), 5.12 (s, 1H), 4.90 (s, 1H), 4.29 (d, J=11.8 Hz, 2H), 4.17 (d, J=12.4 Hz, 1H), 4.11-4.05 (m, 1H), 3.61-3.49 (m, 2H), 3.31-3.22 (m, 2H), 3.14-3.07 (m, 1H); MS (ESI) m/z: 477.1 [M+H⁺].

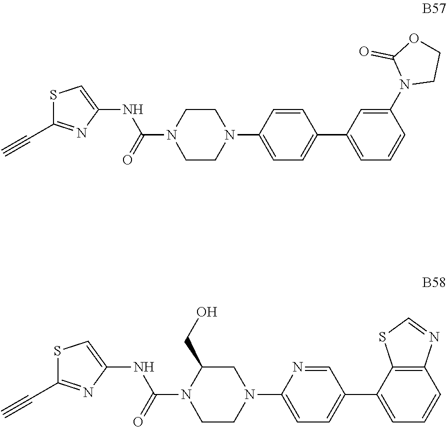 [SMILES: C#Cc1nc(NC(=O)N2CCN(c3ccc(-c4cccc(N5CCOC5=O)c4)cc3)CC2)cs1 ; C#Cc1nc(NC(=O)N2CCN(c3ccc(-c4cccc5ncsc45)cn3)C[C@@H]2CO)cs1]

(R)-4-(5-(Benzo[d]thiazol-7-yl)-3-cyanopyridin-2-yl)-N-(2-ethynylthiazol-4-yl)-2-(hydroxymethyl)piperazine-1-carboxamide B59. ¹H NMR (400 MHz, DMSO-d₆) δ 9.77 (s, 1H), 9.48 (s, 1H), 8.78 (d, J=2.5 Hz, 1H), 8.43 (d, J=2.5 Hz, 1H), 8.13 (dd, J=7.7, 1.5 Hz, 1H), 7.68 (t, J=7.6 Hz, 1H), 7.64 (dd, J=7.4, 1.4 Hz, 1H), 7.43 (s, 1H), 5.14 (t, J=4.6 Hz, 1H), 4.90 (s, 1H), 4.40-4.27 (m, 2H), 4.22 (dd, J=14.2, 8.7 Hz, 1H), 4.07 (dd, J=14.9, 8.4 Hz, 1H), 3.65-3.60 (m, 1H), 3.58 (dd, J=8.7, 4.3 Hz, 2H), 3.41 (d, J=9.1 Hz, 2H); MS (ESI) m/z: 502.1 [M+H⁺].

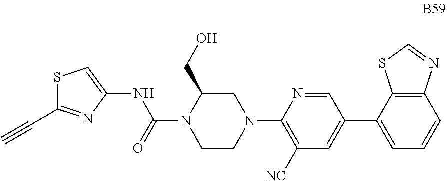 [SMILES: C#Cc1nc(NC(=O)N2CCN(c3ncc(-c4cccc5ncsc45)cc3C#N)C[C@@H]2CO)cs1]

### Example 2

### Preparation of 2-ethynyl-N-(quinolin-2-ylmethyl)thiazole-4-carboxamide C44

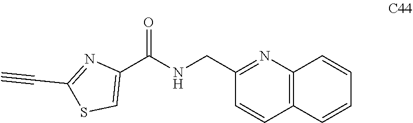 [SMILES: C#Cc1nc(C(=O)NCc2ccc3ccccc3n2)cs1]

Compound C44 was synthesized as shown in Scheme 3.

Ethyl 2-((trimethylsilyl)ethynyl)thiazole-4-carboxylate 11. To a mixture of compound 10 (10.6 g, 45.0 mmol), Pd(PPh₃)₂Cl₂ (2.0 g, 3.5 mmol), and CuI (4.3 g, 22.5 mmol) under argon was added ethynyltrimethylsilane (13.2 g, 135.0 mmol) and DIPEA (8.7 g, 67.5 mmol) in THF (200 mL). After stirred at 50° C. for 2 h, the reaction mixture was concentrated and purified by silica gel chromatography (PE/EA: 10/1) to yield compound 11 (5.8 g) in 51% yield. LCMS (ESI) m/z: 254.1 [M+H⁺].

2-Ethynylthiazole-4-carboxylic acid 12. To a mixture of compound 11 (5.8 g, 22.8 mmol) in THF (30 mL) and H₂O (10 mL) was added LiOH (1.5 g). After stirred at room temperature for 2 h, the reaction mixture was concentrated and extracted with EA (40 mL×6). The organic layers were combined, washed with brine (30 mL×2), dried over anhydrous Na₂SO₄, concentrated, and treated with (PE/EA: 100/1) to yield compound 12 (3.5 g) in a quantitative yield. LCMS (ESI) m/z: 154.1 [M+H⁺].

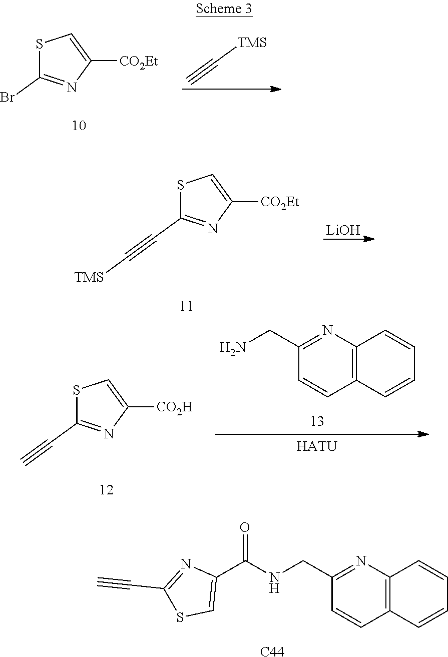

2-Ethynyl-N-(quinolin-2-ylmethyl)thiazole-4-carboxamide C44. To a mixture of compound 12 (40.3 mg, 0.26 mmol) in DMF (5 mL) was added compound 13 (49.9 mg, 0.32 mmol), DIPEA (51.0 mg, 0.40 mmol), and HATU (120.2 mg, 0.32 mmol). After stirred at room temperature for 0.5 h, the reaction mixture was concentrated under reduced pressure and purified by prep-HPLC to yield compound C44 (42.1 mg) in 54.0% yield. LCMS [M+1]⁺=293.1. ¹H NMR (400 MHz, DMSO-d₆) δ 9.31 (t, J=5.8 Hz, 1H), 8.46-8.38 (m, 2H), 8.00 (dd, J=8.1, 3.4 Hz, 2H), 7.80 (t, J=7.6 Hz, 1H), 7.62 (t, J=7.5 Hz, 1H), 7.56 (d, J=8.5 Hz, 1H), 5.08 (s, 1H), 4.77 (d, J=6.0 Hz, 2H); MS (ESI) m/z: 293.1 [M+H⁺].

The following compounds were prepared similarly.

2-Ethynyl-N-(4-(6-fluorobenzo[d]thiazol-5-yl)phenethyl)thiazole-4-carboxamide C1. ¹H NMR (400 MHz, DMSO-d₆) δ 9.39 (s, 1H), 8.77-8.59 (m, 1H), 8.37 (s, 1H), 8.12 (d, J=4.4 Hz, 1H), 8.10 (d, J=4.4 Hz, 1H), 7.56 (dd, J=13.1, 5.9 Hz, 3H), 7.50-7.36 (m, 2H), 5.05 (s, 1H), 3.57 (dd, J=14.3, 6.5 Hz, 2H), 2.95 (t, J=7.4 Hz, 2H).

N-(4-(Benzo[d]thiazol-7-yl)-3-fluorophenethyl)-2-ethynylthiazole-4-carboxamide C2. ¹H NMR (400 MHz, DMSO-d₆) δ 9.44 (s, 1H), 8.72 (t, J=5.9 Hz, 1H), 8.37 (d, J=4.3 Hz, 1H), 8.14 (dd, J=8.2, 1.0 Hz, 1H), 7.66 (dd, J=8.1, 7.5 Hz, 1H), 7.58-7.49 (m, 2H), 7.30 (dd, J=11.6, 1.3 Hz, 1H), 7.24 (dd, J=7.8, 1.5 Hz, 1H), 5.05 (d, J=2.4 Hz, 1H), 3.59 (dd, J=13.4, 7.0 Hz, 3H), 2.97 (t, J=7.2 Hz, 3H); MS (ESI) m/z: 408.1 [M+H⁺].

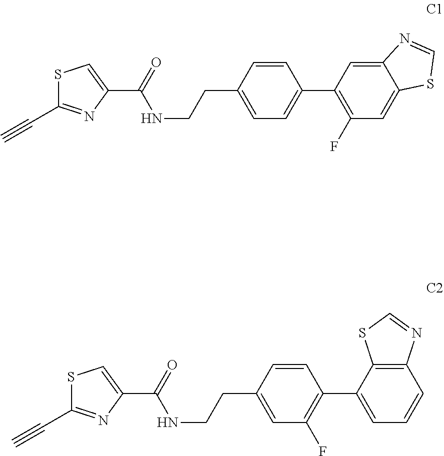 [SMILES: C#Cc1nc(C(=O)NCCc2ccc(-c3cc4ncsc4cc3F)cc2)cs1 ; C#Cc1nc(C(=O)NCCc2ccc(-c3cccc4ncsc34)c(F)c2)cs1]

7-(4-(2-(2-Ethynylthiazole-4-carboxamido)ethyl)phenyl)benzo[d]thiazole-6-carboxamide C3. ¹H NMR (400 MHz, DMSO-d₆) δ 9.44 (s, 1H), 8.72 (t, J=5.9 Hz, 1H), 8.38 (s, 1H), 8.09 (d, J=8.3 Hz, 1H), 7.63 (d, J=8.3 Hz, 2H), 7.45 (d, J=8.2 Hz, 2H), 7.37 (s, 1H), 7.34 (d, J=3.5 Hz, 2H), 5.05 (s, 1H), 3.60-3.54 (m, 2H), 2.98-2.87 (m, 2H); MS (ESI) m/z: 433.0 [M+H⁺].

2-Ethynyl-N-(2-(3′-(methylcarbamoyl)-[1,1′-biphenyl]-4-yl)ethyl)thiazole-4-carboxamide C4. ¹H NMR (400 MHz, DMSO-d₆) δ 8.66 (t, J=5.9 Hz, 1H), 8.54 (d, J=4.5 Hz, 1H), 8.36 (s, 1H), 8.09 (t, J=1.6 Hz, 1H), 7.79 (dd, J=7.8, 1.7 Hz, 2H), 7.66 (d, J=8.2 Hz, 2H), 7.53 (t, J=7.7 Hz, 1H), 7.35 (d, J=8.2 Hz, 2H), 5.04 (s, 1H), 3.53 (dd, J=14.1, 6.5 Hz, 2H), 2.90 (t, J=7.3 Hz, 2H), 2.81 (d, J=4.5 Hz, 3H); MS (ESI) m/z: 390.2 M+H⁺.

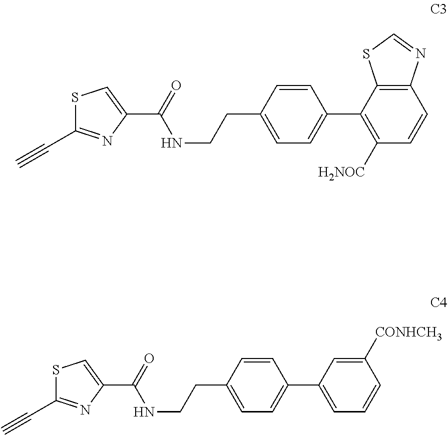

2-Ethynyl-N-(4-(6-fluorobenzo[d]thiazol-7-yl)phenethyl)thiazole-4-carboxamide C5. ¹H NMR (400 MHz, DMSO-d₆) δ 9.41 (d, J=7.7 Hz, 1H), 8.70 (t, J=5.9 Hz, 1H), 8.38 (s, 1H), 8.19 (dd, J=8.8, 6.6 Hz, 2H), 7.58 (dd, J=8.2, 1.7 Hz, 2H), 7.38 (d, J=8.2 Hz, 2H), 5.05 (s, 1H), 3.56 (dd, J=14.4, 6.3 Hz, 2H), 2.93 (t, J=7.5 Hz, 2H).

N-(2-(2′-(N,N-Dimethylsulfamoyl)-[1,1′-biphenyl]-4-yl)ethyl)-2-ethynylthiazole-4-carboxamide C6. ¹H NMR (400 MHz, DMSO-d₆) δ 8.59 (t, J=5.9 Hz, 1H), 8.35 (s, 1H), 7.95 (dd, J=7.9, 1.3 Hz, 1H), 7.67 (dd, J=7.5, 6.1 Hz, 1H), 7.59 (dd, J=7.7, 6.3 Hz, 1H), 7.33-7.31 (m, 1H), 7.27 (d, J=2.1 Hz, 4H), 5.04 (s, 1H), 3.54 (d, J=7.6 Hz, 2H), 2.90 (d, J=7.1 Hz, 2H), 2.27 (s, 6H); MS (ESI) m/z: 440.2 [M+H⁺].

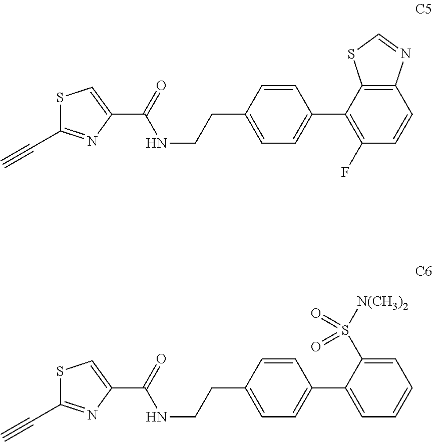 [SMILES: C#Cc1nc(C(=O)NCCc2ccc(-c3c(F)ccc4ncsc34)cc2)cs1 ; C#Cc1nc(C(=O)NCCc2ccc(-c3ccccc3S(=O)(=O)N(C)C)cc2)cs1]

Methyl 2-(2-ethynylthiazole-4-carboxamido)-5-(4-(2-(2-ethynylthiazole-4-carboxamido)ethyl)phenyl)benzo[d]thiazole-7-carboxylate C7.

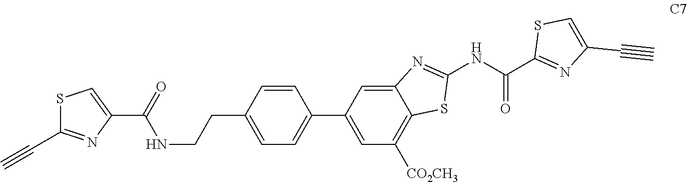 [SMILES: C#Cc1nc(C(=O)NCCc2ccc(-c3cc(C(=O)OC)c4sc(NC(=O)c5nc(CC)cs5)nc4c3)cc2)cs1]

7-(4-(2-(2-Ethynylthiazole-4-carboxamido)ethyl)phenyl)benzo[d]thiazole-5-carboxamide C8. ¹H NMR (400 MHz, DMSO-d₆) δ 9.53 (s, 1H), 8.71 (t, J=5.9 Hz, 1H), 8.59 (d, J=1.5 Hz, 1H), 8.37 (s, 1H), 8.26 (s, 1H), 8.07 (d, J=1.4 Hz, 1H), 7.71 (d, J=8.2 Hz, 2H), 7.56 (s, 1H), 7.45 (d, J=8.2 Hz, 2H), 5.04 (s, 1H), 3.57 (dd, J=14.2, 6.5 Hz, 2H), 2.96 (t, J=7.4 Hz, 2H); MS (ESI) m/z: 433.1 [M+H⁺].

7-(4-(2-(2-Ethynylthiazole-4-carboxamido)ethyl)phenyl)-N-methylbenzo[d]-thiazole-5-carboxamide C9. ¹H NMR (400 MHz, DMSO-d₆) δ 9.53 (s, 1H), 8.71 (dd, J=11.5, 5.4 Hz, 2H), 8.54 (d, J=1.5 Hz, 1H), 8.37 (s, 1H), 8.03 (d, J=1.2 Hz, 1H), 7.71 (d, J=8.2 Hz, 2H), 7.45 (d, J=8.2 Hz, 2H), 5.05 (s, 1H), 3.57 (dd, J=14.2, 6.5 Hz, 2H), 2.96 (t, J=7.3 Hz, 2H), 2.85 (d, J=4.5 Hz, 3H); MS (ESI) m/z: 447.1 [M+H⁺].

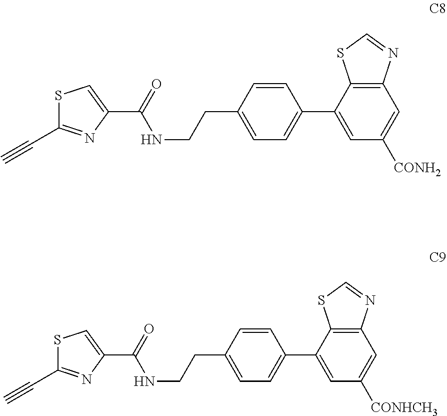

Methyl 7-(4-(2-(2-ethynylthiazole-4-carboxamido)ethyl)phenyl)benzo[d]thiazole-5-carboxylate C10. ¹H NMR (400 MHz, DMSO-d₆) δ 9.58 (s, 1H), 8.71 (t, J=5.9 Hz, 1H), 8.59 (d, J=1.4 Hz, 1H), 8.37 (s, 1H), 8.05 (d, J=1.0 Hz, 1H), 7.70 (d, J=8.0 Hz, 2H), 7.45 (d, J=8.2 Hz, 2H), 5.05 (s, 1H), 3.94 (s, 3H), 3.57 (dd, J=14.3, 6.4 Hz, 2H), 2.96 (t, J=7.4 Hz, 2H); MS (ESI) m/z: 448.1 [M+H⁺].

2-Ethynyl-N-(4-(quinoxalin-5-yl)phenethyl)thiazole-4-carboxamide C11. ¹H NMR (400 MHz, DMSO-d₆) δ 8.97 (dd, J=11.6, 1.8 Hz, 2H), 8.71 (t, J=5.9 Hz, 1H), 8.38 (s, 1H), 8.11 (dd, J=8.2, 1.6 Hz, 1H), 8.01-7.79 (m, 2H), 7.67-7.51 (m, 2H), 7.36 (d, J=8.2 Hz, 2H), 5.05 (s, 1H), 3.57 (dd, J=14.5, 6.3 Hz, 2H), 2.94 (t, J=7.4 Hz, 2H); MS (ESI) m/z: 385.1 [M+H⁺].

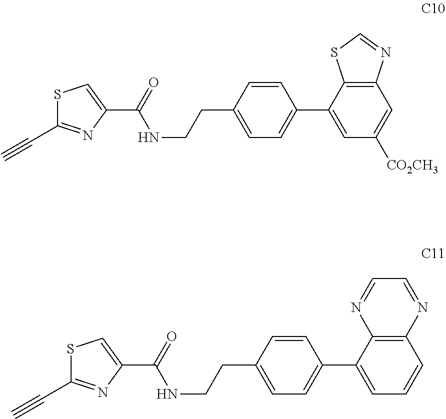 [SMILES: C#Cc1nc(C(=O)NCCc2ccc(-c3cc(C(=O)OC)cc4ncsc34)cc2)cs1 ; C#Cc1nc(C(=O)NCCc2ccc(-c3cccc4nccnc34)cc2)cs1]

2-Ethynyl-N-(2-(2′-(methylcarbamoyl)-[1,1′-biphenyl]-4-yl)ethyl)thiazole-4-carboxamide C12. ¹H NMR (400 MHz, DMSO-d₆) δ 8.65 (t, J=5.9 Hz, 1H), 8.36 (s, 1H), 7.99 (q, J=4.5 Hz, 1H), 7.49-7.45 (m, 1H), 7.39-7.37 (m, 3H), 7.33-7.30 (m, 2H), 7.27-7.24 (m, 2H), 5.04 (s, 1H), 3.53-3.51 (m, 2H), 2.88 (t, J=7.5 Hz, 2H), 2.55 (d, J=4.6 Hz, 3H); MS (ESI) m/z: 390.1 [M+H⁺].

2-Ethynyl-N-(4-(3-oxoisoindolin-4-yl)phenethyl)thiazole-4-carboxamide C13. ¹H NMR (400 MHz, DMSO-d₆) δ 8.70 (t, J=5.9 Hz, 1H), 8.48 (s, 1H), 8.37 (s, 1H), 7.61 (t, J=7.5 Hz, 1H), 7.53 (d, J=6.8 Hz, 1H), 7.46-7.41 (m, 2H), 7.33 (d, J=6.8 Hz, 1H), 7.26 (d, J=8.2 Hz, 2H), 5.04 (s, 1H), 4.36 (s, 2H), 3.54 (dd, J=14.9, 6.2 Hz, 3H), 2.90 (dd, J=9.9, 5.2 Hz, 2H); MS (ESI) m/z: 388.0 [M+H⁺].

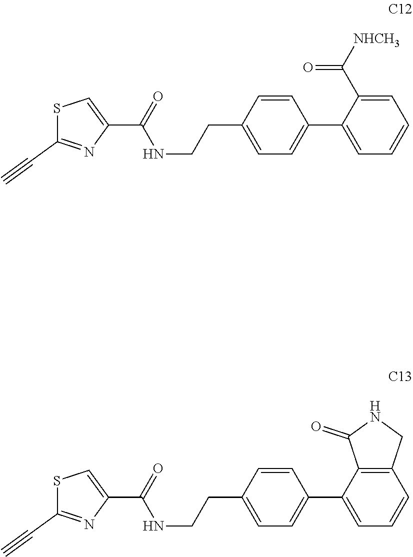 [SMILES: C#Cc1nc(C(=O)NCCc2ccc(-c3ccccc3C(=O)NC)cc2)cs1 ; C#Cc1nc(C(=O)NCCc2ccc(-c3cccc4c3C(=O)NC4)cc2)cs1]

N-(4-(2,3-Dioxoindolin-4-yl)phenethyl)-2-ethynylthiazole-4-carboxamide C14. ¹H NMR (400 MHz, DMSO-d₆) δ 11.10 (s, 1H), 8.70 (t, J=5.9 Hz, 1H), 8.37 (s, 1H), 7.59 (t, J=7.8 Hz, 1H), 7.49 (d, J=8.2 Hz, 2H), 7.31 (d, J=8.2 Hz, 2H), 7.01 (dd, J=7.8, 0.7 Hz, 1H), 6.87 (dd, J=7.8, 0.7 Hz, 1H), 5.04 (s, 1H), 3.63-3.49 (m, 2H), 2.92 (t, J=7.5 Hz, 2H); MS (ESI) m/z: 402.0 [M+H⁺].

2-Ethynyl-N-(2-(2′-(methylsulfonyl)-[1,1′-biphenyl]-4-yl)ethyl)thiazole-4-carboxamide C15. ¹H NMR (400 MHz, DMSO-d₆) δ 8.67 (t, J=5.9 Hz, 1H), 8.35 (s, 1H), 8.09 (dd, J=8.0, 1.3 Hz, 1H), 7.75 (td, J=7.5, 1.4 Hz, 1H), 7.66 (td, J=7.7, 1.4 Hz, 1H), 7.40 (dd, J=7.6, 1.2 Hz, 1H), 7.37-7.27 (m, 4H), 5.05 (s, 1H), 3.55 (dd, J=14.5, 6.4 Hz, 2H), 2.92 (t, J=7.5 Hz, 2H), 2.73 (s, 3H); MS (ESI) m/z: 411.1 [M+H⁺].

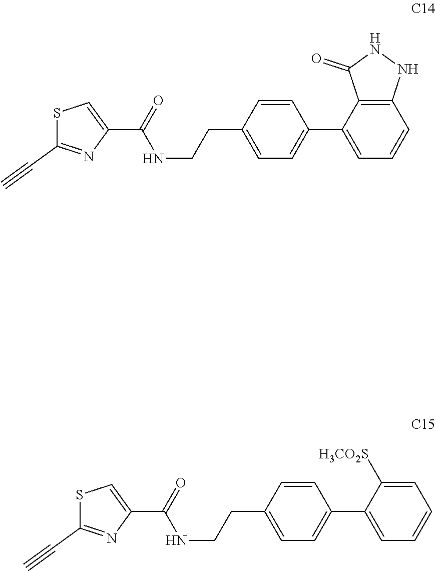

2-Ethynyl-N-(4-(1-hydroxyisoquinolin-8-yl)phenethyl)thiazole-4-carboxamide C16. ¹H NMR (400 MHz, DMSO-d₆) δ 10.90 (d, J=5.4 Hz, 1H), 8.70 (d, J=5.9 Hz, 1H), 8.38 (s, 1H), 7.79-7.54 (m, 2H), 7.27-7.03 (m, 6H), 6.56 (dd, J=7.1, 1.4 Hz, 1H), 5.05 (s, 1H), 3.54 (dd, J=15.3, 6.1 Hz, 2H), 3.03-2.78 (m, 2H); MS (ESI) m/z: 400.1 [M+H⁺].

2-Ethynyl-N-(4-(1-oxoisoindolin-4-yl)phenethyl)thiazole-4-carboxamide C17. ¹H NMR (400 MHz, DMSO-d₆) δ 8.78-8.59 (m, 2H), 8.37 (s, 1H), 7.68 (dd, J=7.3, 1.2 Hz, 1H), 7.65 (d, J=6.5 Hz, 1H), 7.59 (d, J=7.4 Hz, 1H), 7.56 (d, J=8.1 Hz, 2H), 7.36 (d, J=8.2 Hz, 2H), 5.05 (s, 1H), 4.50 (s, 2H), 3.55 (dd, J=14.3, 6.4 Hz, 2H), 2.92 (t, J=7.4 Hz, 2H); MS (ESI) m/z 38. [M+H⁺].

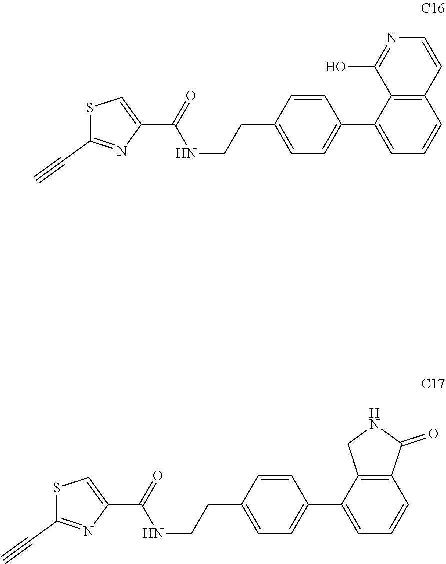 [SMILES: C#Cc1nc(C(=O)NCCc2ccc(-c3cccc4ccnc(O)c34)cc2)cs1 ; C#Cc1nc(C(=O)NCCc2ccc(-c3cccc4c3CNC4=O)cc2)cs1]

2-Ethynyl-N-(4-(3-oxo-2,3-dihydro-1H-inden-4-yl)phenethyl)thiazole-4-carboxamide C18. ¹H NMR (400 MHz, DMSO-d₆) δ 8.70 (t, J=5.9 Hz, 1H), 8.37 (s, 1H), 7.66 (t, J=7.5 Hz, 1H), 7.54 (dd, J=7.6, 0.8 Hz, 1H), 7.41-7.29 (m, 2H), 7.25 (dd, J=7.4, 5.8 Hz, 3H), 5.05 (s, 1H), 3.54 (dd, J=14.9, 6.2 Hz, 2H), 3.17-3.04 (m, 2H), 2.98-2.83 (m, 2H), 2.67-2.56 (m, 2H); MS (ESI) m/z: 387.1 [M+H⁺].

2-Ethynyl-N-(4-(thiazolo[4,5-c]pyridin-7-yl)phenethyl)thiazole-4-carboxamide C19. ¹H NMR (400 MHz, DMSO-d₆) δ 9.64 (s, 1H), 9.42 (s, 1H), 8.73 (s, 2H), 8.37 (s, 1H), 7.74 (d, J=8.3 Hz, 2H), 7.48 (d, J=8.3 Hz, 2H), 5.05 (s, 1H), 3.57 (dd, J=14.2, 6.5 Hz, 3H), 2.97 (t, J=7.3 Hz, 2H); MS (ESI) m/z: 391.1 [M+H⁺].

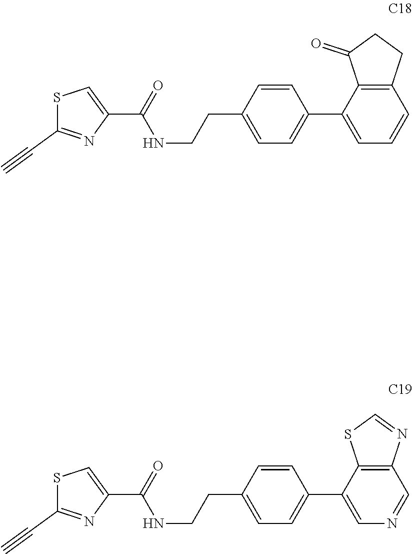 [SMILES: C#Cc1nc(C(=O)NCCc2ccc(-c3cccc4c3C(=O)CC4)cc2)cs1 ; C#Cc1nc(C(=O)NCCc2ccc(-c3cncc4ncsc34)cc2)cs1]

2-Ethynyl-N-(2-(2′-(N-methylsulfamoyl)-[1,1′-biphenyl]-4-yl)ethyl)thiazole-4-carboxamide C20. ¹H NMR (400 MHz, DMSO-d₆) δ 8.69 (t, J=5.9 Hz, 1H), 8.35 (s, 1H), 7.92 (dd, J=7.9, 1.3 Hz, 1H), 7.64 (dd, J=7.5, 1.4 Hz, 1H), 7.59 (dd, J=7.7, 1.4 Hz, 1H), 7.35-7.28 (m, 3H), 7.25 (d, J=8.2 Hz, 2H), 6.76 (d, J=4.9 Hz, 1H), 5.04 (s, 1H), 3.54 (dd, J=14.5, 6.4 Hz, 2H), 2.90 (t, J=7.4 Hz, 2H), 2.31 (d, J=4.9 Hz, 3H); MS (ESI) m/z: 426.1 [M+H⁺].

2-Ethynyl-N-(4-(isoquinolin-5-yl)phenethyl)thiazole-4-carboxamide C21. ¹H NMR (400 MHz, DMSO-d₆) δ 9.56 (s, 1H), 8.72 (s, 1H), 8.53 (d, J=6.2 Hz, 1H), 8.38 (s, 1H), 8.33-8.24 (m, 1H), 7.90-7.78 (m, 3H), 7.49-7.40 (m, 4H), 5.06 (s, 1H), 3.59 (dd, J=14.5, 6.4 Hz, 2H), 3.01-2.93 (m, 2H); MS (ESI) m/z: 384.1 [M+H⁺].

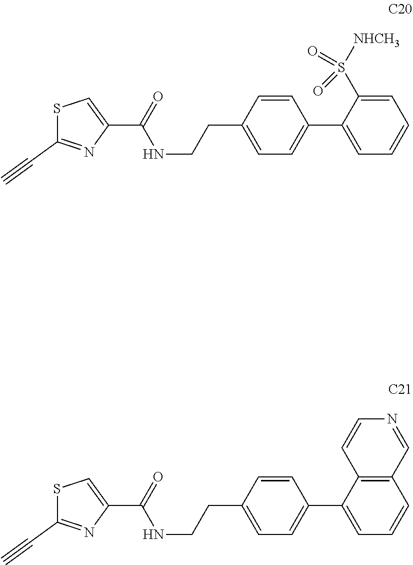 [SMILES: C#Cc1nc(C(=O)NCCc2ccc(-c3ccccc3S(=O)(=O)NC)cc2)cs1 ; C#Cc1nc(C(=O)NCCc2ccc(-c3cccc4cnccc34)cc2)cs1]

2-Ethynyl-N-(4-(2-methyl-1-oxoisoindolin-4-yl)phenethyl)thiazole-4-carboxamide C22. ¹H NMR (400 MHz, DMSO-d₆) δ 8.68 (t, J=5.9 Hz, 1H), 8.37 (s, 1H), 7.65 (ddd, J=10.2, 7.5, 1.2 Hz, 2H), 7.61-7.52 (m, 3H), 7.36 (t, J=6.7 Hz, 2H), 5.05 (s, 1H), 4.60 (s, 2H), 3.54 (dd, J=14.4, 6.4 Hz, 2H), 3.07 (s, 3H), 2.92 (t, J=7.4 Hz, 2H); MS (ESI) m/z: 402.1 [M+H⁺].

2-Ethynyl-N-(4-(quinolin-5-yl)phenethyl)thiazole-4-carboxamide C23. ¹H NMR (400 MHz, DMSO-d₆) δ 8.99 (dd, J=4.3, 1.6 Hz, 1H), 8.72 (t, J=5.9 Hz, 1H), 8.38 (s, 1H), 8.29 (d, J=8.3 Hz, 1H), 8.08 (d, J=8.5 Hz, 1H), 7.87 (dd, J=8.5, 7.2 Hz, 1H), 7.64-7.55 (m, 2H), 7.46-7.41 (m, 5H), 5.05 (s, 1H), 3.58 (dd, J=14.6, 6.3 Hz, 2H), 3.02-2.93 (m, 2H); MS (ESI) m/z: 384.1 [M+H⁺].

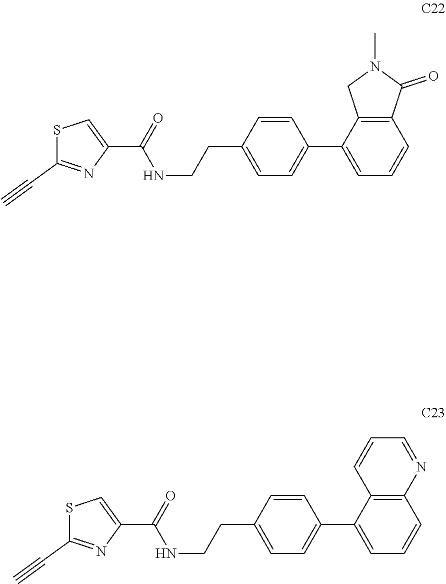 [SMILES: C#Cc1nc(C(=O)NCCc2ccc(-c3cccc4c3CN(C)C4=O)cc2)cs1 ; C#Cc1nc(C(=O)NCCc2ccc(-c3cccc4ncccc34)cc2)cs1]

2-Ethynyl-N-(4-(quinazolin-5-yl)phenethyl)thiazole-4-carboxamide C24. ¹H NMR (400 MHz, DMSO-d₆) δ 9.38 (s, 1H), 9.34 (s, 1H), 873 (t, 1H, J=6.0 Hz), 8.38 (s, 1H), 8.03-8.12 (m, 2H), 7.72 (dd, 1=1.2 Hz, 6.8 Hz, 1H), 7.43-7.57 (m, 4H), 5.05 (s, 1H), 3.55-3.64 (m, 2H), 2.98 (t, 1H, J=6.8 Hz); MS (ESI) m/z: 385.1 [M+H⁺].

N-(4-(Benzo[d]thiazol-5-yl)phenethyl)-2-ethynylthiazole-4-carboxamide C25. ¹H NMR (400 MHz, DMSO-d₆) δ 9.43 (s, 1H), 8.68 (t, J=5.8 Hz, 1H), 8.39-8.30 (m, 2H), 8.24 (d, J=8.4 Hz, 1H), 7.80 (dd, J=8.4, 1.7 Hz, 1H), 7.73 (d, J=8.2 Hz, 2H), 7.36 (d, J=8.2 Hz, 2H), 5.04 (s, 1H), 3.54 (dd, J=14.1, 6.5 Hz, 2H), 2.91 (t, J=7.4 Hz, 2H); MS (ESI) m/z: 390.1 [M+H⁺].

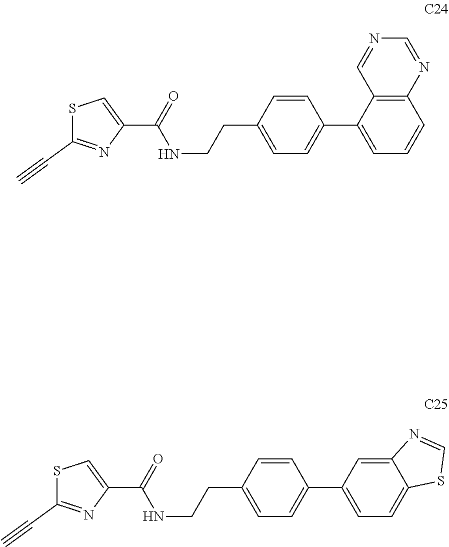 [SMILES: C#Cc1nc(C(=O)NCCc2ccc(-c3cccc4ncncc34)cc2)cs1 ; C#Cc1nc(C(=O)NCCc2ccc(-c3ccc4scnc4c3)cc2)cs1]

2-Ethynyl-N-(3-(1-methyl-2-oxoindolin-4-yl)phenethyl)thiazole-4-carboxamide C26. ¹H NMR (400 MHz, DMSO-d₆) δ 8.63 (t, J=5.8 Hz, 1H), 8.34 (s, 1H), 7.42-7.35 (m, 4H), 7.25-7.22 (m, 1H), 7.06-7.03 (m, 1H), 6.99-6.96 (m, 1H), 5.03 (s, 1H), 3.65 (s, 2H), 3.57-3.52 (m, 2H), 3.15 (s, 3H), 2.92 (t, J=7.2 Hz, 2H); MS (ESI) m/z: 402.1 [M+H⁺].

2-Ethynyl-N-(3-(2-oxoindolin-4-yl)phenethyl)thiazole-4-carboxamide C27. ¹H NMR (400 MHz, DMSO-d₆) δ 10.46 (s, 1H), 8.63 (t, J=5.8 Hz, 1H), 8.35 (s, 1H), 7.41-7.34 (m, 3H), 7.27-7.22 (m, 2H), 6.98-6.95 (m, 1H), 6.83-6.80 (m, 1H), 5.03 (s, 1H), 3.59-3.52 (m, 4H), 2.92 (t, 2H); MS (ESI) m/z: 388.1 [M+H⁺].

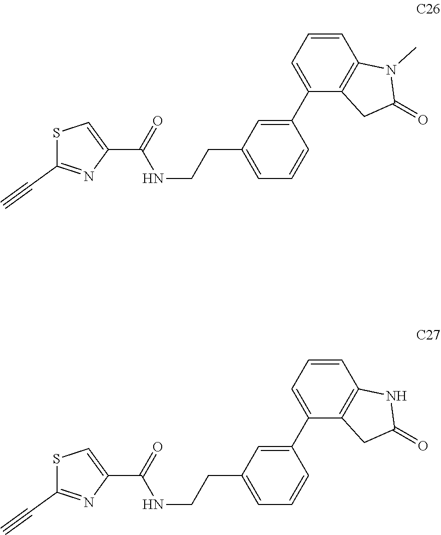 [SMILES: C#Cc1nc(C(=O)NCCc2cccc(-c3cccc4c3CC(=O)N4C)c2)cs1 ; C#Cc1nc(C(=O)NCCc2cccc(-c3cccc4c3CC(=O)N4)c2)cs1]

N-(4-(1H-Indol-4-yl)benzyl)-2-ethynylthiazole-4-carboxamide C28. ¹H NMR (400 MHz, DMSO-d₆) δ 12.24 (s, 1H), 9.20 (t, J=6.0 Hz, 1H), 8.41 (s, 1H), 7.61 (d, J=8.0 Hz, 1H), 7.44-7.38 (m, 4H), 7.16 (t, J=8.0 Hz, 1H), 7.05 (d, J=8.0 Hz, 1H), 6.54-6.53 (m, 1H), 5.05 (s, 1H), 2.96 (d, J=8.0 Hz, 2H); MS (ESI) m/z: 358.1 [M+H⁺].

2-Ethynyl-N-(3-(2-methyl-2H-indazol-4-yl)phenethyl)thiazole-4-carboxamide C29. ¹H NMR (400 MHz, DMSO-d₆) δ 8.66 (t, J=6.0 Hz, 1H), 8.47 (s, 1H), 8.35 (s, 1H), 7.59-7.54 (m, 3H), 7.43 (t, J=8.0 Hz, 1H), 7.32-7.26 (m, 2H), 7.11 (d, J=4.0 Hz, 1H), 5.04 (s, 1H), 4.18 (s, 3H), 3.58 (q, J=6.0 Hz, 2H), 2.96 (t, J=6.0 Hz, 2H); MS (ESI) m/z: 387.1 [M+H⁺].

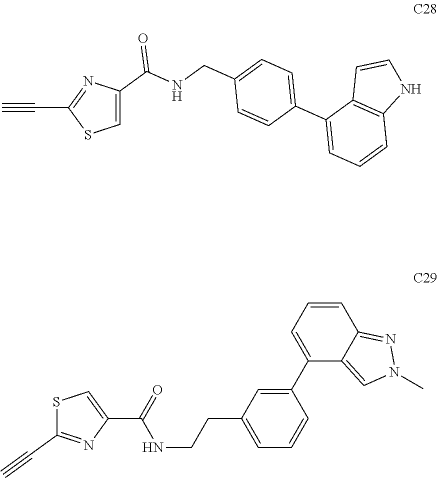 [SMILES: C#Cc1nc(C(=O)NCc2ccc(-c3cccc4[nH]ccc34)cc2)cs1 ; C#Cc1nc(C(=O)NCCc2cccc(-c3cccc4nn(C)cc34)c2)cs1]

2-Ethynyl-N-(3-(1-methyl-1H-indazol-4-yl)phenethyl)thiazole-4-carboxamide C30. ¹H NMR (400 MHz, DMSO-d₆) δ 8.67 (t, J=6.0 Hz, 1H), 8.35 (s, 1H), 8.11 (s, 1H), 7.63 (d, J=8.0 Hz, 1H), 7.56 (d, J=8.0 Hz, 1H), 7.48-7.43 (m, 2H), 7.29 (d, J=8.0 Hz, 1H), 7.21 (d, J=8.0 Hz, 1H), 5.03 (s, 1H), 4.09 (s, 3H), 3.58 (q, J=6.0 Hz, 2H), 2.96 (t, J=6.0 Hz, 2H); MS (ESI) m/z: 387.1 [M+H⁺].

N-(4-(Benzo[d]thiazol-4-yl)phenethyl)-2-ethynylthiazole-4-carboxamide C31. ¹H NMR (400 MHz, DMSO-d₆) δ 9.41 (s, 1H), 8.70 (t, J=5.9 Hz, 1H), 8.37 (s, 1H), 8.16 (dd, J=7.9, 1.2 Hz, 1H), 7.84-7.75 (m, 2H), 7.63 (dd, J=7.5, 1.3 Hz, 1H), 7.57 (t, J=7.7 Hz, 1H), 7.36 (d, J=8.2 Hz, 2H), 5.04 (s, 1H), 3.56 (dd, J=14.4, 6.4 Hz, 2H), 2.93 (t, J=7.4 Hz, 2H); MS (ESI) m/z: 390.0 [M+H⁺].

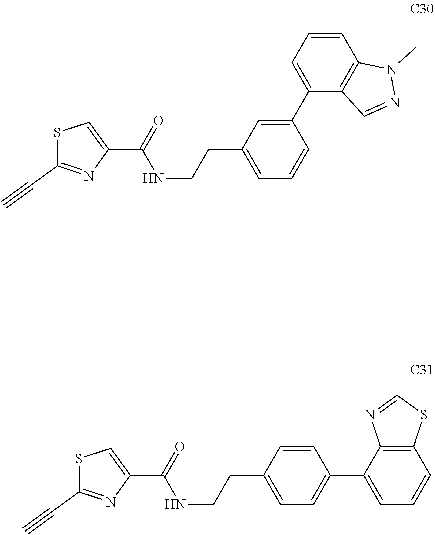

2-Ethynyl-N-(4-(2-methyl-2H-indazol-4-yl)phenethyl)thiazole-4-carboxamide C32. ¹H NMR (400 MHz, DMSO-d₆) δ 8.15 (s, 1H), 8.06 (s, 1H), 7.69 (d, J=8.7 Hz, 1H), 7.65-7.58 (m, 2H), 7.39 (dt, J=14.8, 11.8 Hz, 4H), 7.20-7.13 (m, 1H), 4.26 (s, 3H), 3.76 (dd, J=14.0, 6.7 Hz, 2H), 3.52 (s, 1H), 3.00 (t, J=7.2 Hz, 2H); MS (ESI) m/z: 387.2 [M+H⁺].

2-Ethynyl-N-(4-(1-methyl-1H-indazol-4-yl)phenethyl)thiazole-4-carboxamide C33. ¹H NMR (400 MHz, DMSO-d₆) δ 8.14 (s, 2H), 7.69-7.63 (m, 2H), 7.47 (dd, J=8.4, 7.0 Hz, 2H), 7.38 (dd, J=8.2, 1.5 Hz, 3H), 7.27-7.21 (m, 2H), 4.13 (s, 3H), 3.92-3.65 (m, 2H), 3.53 (s, 1H), 3.01 (t, J=7.2 Hz, 2H); MS (ESI) m/z: 387.2 [M+H⁺].

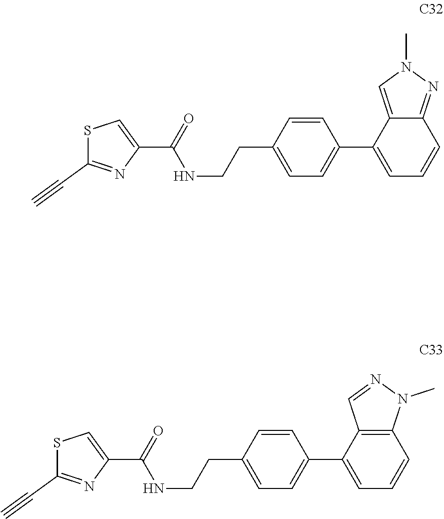 [SMILES: C#Cc1nc(C(=O)NCCc2ccc(-c3cccc4nn(C)cc34)cc2)cs1 ; C#Cc1nc(C(=O)NCCc2ccc(-c3cccc4c3cnn4C)cc2)cs1]

N-(4-(Benzo[d]thiazol-7-yl)phenethyl)-2-ethynylthiazole-4-carboxamide C34. ¹H NMR (400 MHz, DMSO-d₆) δ 9.45 (s, 1H), 8.70 (t, J=5.9 Hz, 1H), 8.37 (s, 1H), 8.09 (dd, J=8.1, 1.1 Hz, 1H), 7.66 (dt, J=7.9, 3.6 Hz, 3H), 7.58 (dd, J=7.4, 0.7 Hz, 1H), 7.42 (d, J=8.2 Hz, 2H), 5.05 (s, 1H), 3.56 (dd, J=14.4, 6.4 Hz, 2H), 2.95 (t, J=7.4 Hz, 2H); MS (ESI) m/z: 390.0 [M+H⁺].

2-Ethynyl-N-(3-(1-methyl-1H-indol-4-yl)phenethyl)thiazole-4-carboxamide C35. ¹H NMR (400 MHz, DMSO-d₆) δ 8.67 (t, J=5.8 Hz, 1H), 8.36 (s, 1H), 7.47 (s, 2H), 7.44-7.41 (m, 2H), 7.33 (d, J=3.1 Hz, 1H), 7.23 (dd, J=7.5, 2.7 Hz, 2H), 7.07 (d, J=7.1 Hz, 1H), 6.49 (d, J=2.7 Hz, 1H), 5.04 (s, 1H), 3.82 (s, 3H), 3.56 (d, J=6.8 Hz, 2H), 2.94 (t, J=7.2 Hz, 2H); MS (ESI) m/z: 386.1 [M+H⁺].

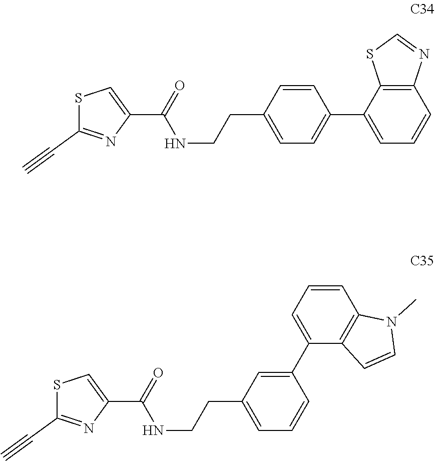 [SMILES: C#Cc1nc(C(=O)NCCc2ccc(-c3cccc4ncsc34)cc2)cs1 ; C#Cc1nc(C(=O)NCCc2cccc(-c3cccc4c3ccn4C)c2)cs1]

N-(3-(1H-Indol-4-yl)phenethyl)-2-ethynylthiazole-4-carboxamide C36. ¹H NMR (400 MHz, DMSO-d₆) δ 11.22 (s, 1H), 8.67 (s, 1H), 8.36 (s, 1H), 7.50-7.47 (m, 2H), 7.42-7.38 (m, 2H), 7.35-7.33 (m, 1H), 7.23 (d, J=7.5 Hz, 1H), 7.17-7.13 (m, 1H), 7.03 (dd, J=7.2, 0.8 Hz, 1H), 6.50 (d, J=2.1 Hz, 1H), 5.04 (s, 1H), 3.57 (d, J=6.8 Hz, 2H), 2.94 (s, 2H); MS (ESI) m/z: 372.1 [M+H⁺].

2-Ethynyl-N-(4-(2-oxoindolin-4-yl)benzyl)thiazole-4-carboxamide C37. ¹H NMR (400 MHz, DMSO-d₆) δ 10.46 (s, 1H), 9.20 (t, J=6.3 Hz, 1H), 8.41 (s, 1H), 7.52 (d, J=8.2 Hz, 2H), 7.38 (d, J=8.2 Hz, 2H), 7.26 (t, J=7.8 Hz, 1H), 6.99 (dd, J=7.9, 0.8 Hz, 1H), 6.81 (d, J=7.4 Hz, 1H), 5.05 (s, 1H), 4.48 (d, J=6.3 Hz, 2H), 3.59 (s, 2H); MS (ESI) m/z: 374.1 [M+H⁺].

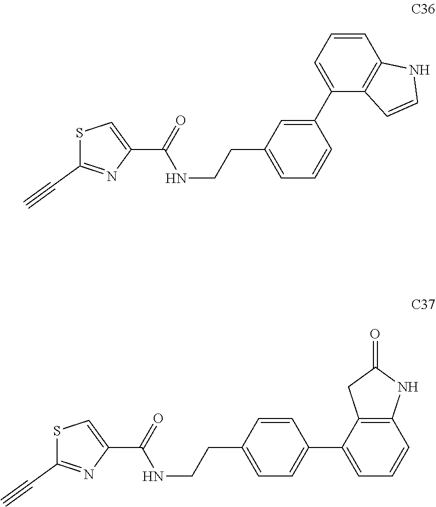 [SMILES: C#Cc1nc(C(=O)NCCc2cccc(-c3cccc4[nH]ccc34)c2)cs1 ; C#Cc1nc(C(=O)NCCc2ccc(-c3cccc4c3CC(=O)N4)cc2)cs1]

2-Ethynyl-N-(3-(pyridin-3-yl)phenethyl)thiazole-4-carboxamide C38. ¹H NMR (400 MHz, DMSO-d₆) δ 9.05 (s, 1H), 8.72 (s, 1H), 8.67 (t, J=6.0 Hz, 1H), 8.44 (d, J=8.0 Hz, 1H), 8.35 (s, 1H), 7.71-7.78 (m, 1H), 7.66-7.62 (m, 2H), 7.47 (t, J=8.0 Hz, 1H), 7.35 (d, J=8.0 Hz, 1H), 5.04 (s, 1H), 3.58 (q, J=6.0 Hz, 2H), 2.96 (t, J=8.0 Hz, 2H); MS (ESI) m/z: 334.4 [M+H⁺].

N-(3-(1H-Indazol-4-yl)phenethyl)-2-ethynylthiazole-4-carboxamide C39. ¹H NMR (400 MHz, DMSO-d₆) δ 13.2 (s, 1H), 8.68 (t, J=6.0 Hz, 1H), 8.35 (s, 1H), 8.13 (s, 1H), 7.54 (t, J=10.0 Hz, 3H), 7.47-7.39 (m, 2H), 7.29 (d, J=8.0 Hz, 1H), 7.18 (d, J=4.0 Hz, 1H), 5.03 (s, 1H), 3.59 (q, J=6.0 Hz, 2H), 2.96 (t, J=8.0 Hz, 2H); MS (ESI) m/z: 373.1 [M+H⁺].

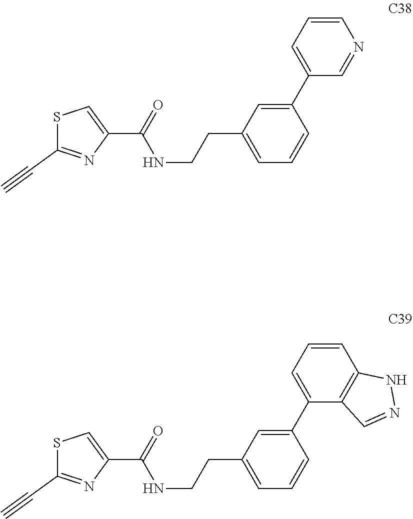 [SMILES: C#Cc1nc(C(=O)NCCc2cccc(-c3cccnc3)c2)cs1 ; C#Cc1nc(C(=O)NCCc2cccc(-c3cccc4[nH]ncc34)c2)cs1]

N-(4-(1H-Indazol-4-yl)benzyl)-2-ethynylthiazole-4-carboxamide C40. ¹H NMR (400 MHz, DMSO-d₆) δ 13.2 (s, 1H), 9.23 (t, J=8.0 Hz, 1H), 8.42 (s, 1H), 8.16 (d, J=4.0 Hz, 1H), 7.69 (d, J=8.0 Hz, 2H), 7.53 (d, J=8.0 Hz, 1H), 7.47 (d, J=8.0 Hz, 2H), 7.44-7.40 (m, 1H), 7.21 (d, J=4.0 Hz, 1H), 5.06 (s, 1H), 4.52 (d, J=8.0 Hz, 2H); MS (ESI) m/z: 359.1 [M+H⁺].

2-Ethynyl-N-(4-(1-methyl-1H-pyrazol-3-yl)phenethyl)thiazole-4-carboxamide C41. ¹H NMR (400 MHz, DMSO-d₆) δ 8.63 (t, J=5.9 Hz, 1H), 8.35 (s, 1H), 7.69 (dd, J=6.4, 1.9 Hz, 3H), 7.24 (d, J=8.2 Hz, 2H), 6.64 (d, J=2.3 Hz, 1H), 5.04 (s, 1H), 3.86 (s, 3H), 3.55-3.48 (m, 2H), 2.85 (t, J=7.4 Hz, 2H); MS (ESI) m/z: 337.1 [M+H⁺].

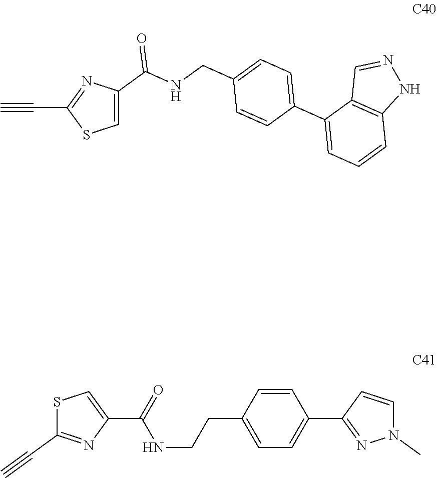 [SMILES: C#Cc1nc(C(=O)NCc2ccc(-c3cccc4[nH]ncc34)cc2)cs1 ; C#Cc1nc(C(=O)NCCc2ccc(-c3ccn(C)n3)cc2)cs1]

N-(4-(Benzo[d]thiazol-6-yl)phenethyl)-2-ethynylthiazole-4-carboxamide C42. ¹H NMR (400 MHz, DMSO-d₆) δ 9.40 (s, 1H), 8.67 (t, J=5.9 Hz, 1H), 8.47 (d, J=1.8 Hz, 1H), 8.36 (s, 1H), 8.14 (d, J=8.5 Hz, 1H), 7.83 (dd, J=8.6, 1.9 Hz, 1H), 7.70 (d, J=8.2 Hz, 2H), 7.36 (d, J=8.2 Hz, 2H), 5.04 (s, 1H), 3.54 (dd, J=14.2, 6.5 Hz, 2H), 2.91 (t, J=7.4 Hz, 2H); MS (ESI) m/z: 390.1 [M+H⁺].

2-Ethynyl-N-(4-(1-methyl-2-oxoindolin-4-yl)phenethyl)thiazole-4-carboxamide C43. ¹H NMR (400 MHz, DMSO-d₆) δ 8.68 (t, J=5.8 Hz, 1H), 8.36 (s, 1H), 7.52 (d, J=8.1 Hz, 2H), 7.46-7.25 (m, 3H), 7.09 (d, J=7.8 Hz, 1H), 6.97 (d, J=7.8 Hz, 1H), 5.04 (s, 1H), 3.67 (s, 3H), 3.53 (dd, J=14.1, 6.6 Hz, 4H), 3.15 (s, 3H), 2.90 (t, J=7.4 Hz, 2H); MS (ESI) m/z: 402.2 [M+H⁺].

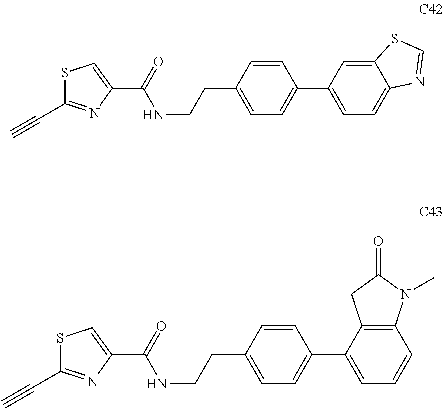 [SMILES: C#Cc1nc(C(=O)NCCc2ccc(-c3ccc4ncsc4c3)cc2)cs1 ; C#Cc1nc(C(=O)NCCc2ccc(-c3cccc4c3CC(=O)N4C)cc2)cs1]

N-(2-Cyanophenethyl)-2-ethynylthiazole-4-carboxamide C45. ¹H NMR (400 MHz, DMSO-d₆) δ 8.73 (t, J=5.6 Hz, 1H), 8.34 (s, 1H), 7.78 (d, J=7.6 Hz, 1H), 7.59-7.67 (m, 1H), 7.38-7.50 (m, 2H), 5.04 (s, 1H), 3.53-3.64 (m, 2H), 3.03-3.12 (m, 2H); MS (ESI) m/z: 282.0 [M+H⁺].

Methyl 2-(3-((2-ethynylthiazole-4-carboxamido)methyl)phenyl)acetate C46. ¹H NMR (400 MHz, DMSO-d₆) δ 9.13 (t, J=6.0 Hz, 1H), 8.39 (s, 1H), 7.28-7.10 (m, 4H), 5.05 (s, 1H), 4.42 (d, J=8.0 Hz, 2H), 3.65 (s, 2H), 3.60 (s, 3H); MS (ESI) m/z: 315.4 [M+H⁺].

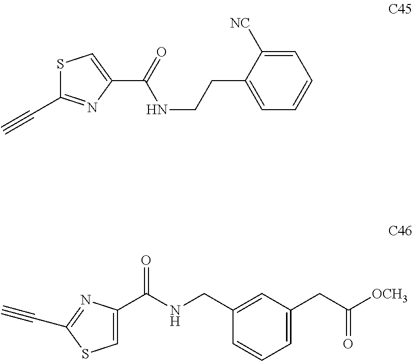 [SMILES: C#Cc1nc(C(=O)NCCc2ccccc2C#N)cs1 ; C#Cc1nc(C(=O)NCc2cccc(CC(=O)OC)c2)cs1]

N-(3-(2-Amino-2-oxoethyl)benzyl)-2-ethynylthiazole-4-carboxamide C47. ¹H NMR (400 MHz, DMSO-d₆) δ 9.11 (t, J=8.0 Hz, 1H), 8.39 (s, 1H), 7.44 (brs, 1H), 7.25-7.12 (m, 4H), 6.85 (brs, 1H), 5.04 (s, 1H), 4.41 (d, J=4.0 Hz, 2H), 3.84 (brs, 2H); MS (ESI) nm/z: 300.1 [M+H⁺].

2-Ethynyl-N-(4-(2-oxoindolin-4-yl)phenethyl)thiazole-4-carboxamide C48. ¹H NMR (400 MHz, DMSO-d₆) δ 10.45 (s, 1H), 8.67 (t, J=5.9 Hz, 1H), 8.36 (s, 1H), 7.50 (d, J=8.2 Hz, 2H), 7.35-7.20 (m, 3H), 7.11-6.93 (m, 1H), 6.80 (d, J=7.3 Hz, 1H), 5.04 (s, 1H), 3.58 (d, J=13.6 Hz, 3H), 3.49 (dd, J=26.7, 20.5 Hz, 8H), 2.89 (t, J=7.4 Hz, 2H); MS (ESI) m/z: 388.3 [M+H⁺].

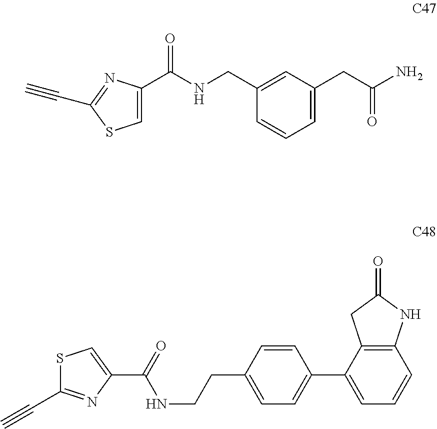 [SMILES: C#Cc1nc(C(=O)NCc2cccc(CC(N)=O)c2)cs1 ; C#Cc1nc(C(=O)NCCc2ccc(-c3cccc4c3CC(=O)N4)cc2)cs1]

N-(4-(1H-Indazol-4-yl)phenethyl)-2-ethynylthiazole-4-carboxamide C49. ¹H NMR (400 MHz, DMSO-d₆) δ 13.21 (s, 1H), 8.70 (t, J=5.9 Hz, 1H), 8.37 (s, 1H), 8.16 (d, J=0.9 Hz, 1H), 7.67 (d, J=8.2 Hz, 2H), 7.53 (d, J=8.4 Hz, 1H), 7.45-7.36 (m, 3H), 7.21 (dd, J=7.0, 0.7 Hz, 1H), 5.04 (s, 1H), 3.65-3.49 (m, 5H), 2.93 (t, J=7.5 Hz, 2H); MS (ESI) nm/z: 373.1 [M+H⁺].

2-Ethynyl-N-(4-(1-methyl-1H-indol-4-yl)phenethyl)thiazole-4-carboxamide C50. MS (ESI) m/z: 386.1 [M+H⁺].

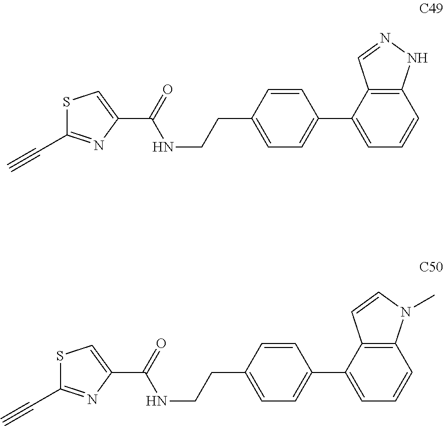 [SMILES: C#Cc1nc(C(=O)NCCc2ccc(-c3cccc4[nH]ncc34)cc2)cs1 ; C#Cc1nc(C(=O)NCCc2ccc(-c3cccc4c3ccn4C)cc2)cs1]

N-(4-(1H-Indol-4-yl)phenethyl)-2-ethynylthiazole-4-carboxamide C51. ¹H NMR (400 MHz, DMSO-d₆) δ 11.23 (s, 1H), 8.69 (t, J=5.9 Hz, 1H), 8.37 (s, 1H), 7.59 (d, J=8.1 Hz, 2H), 7.38 (dt, J=14.7, 5.3 Hz, 4H), 7.20-7.11 (m, 1H), 7.06 (dd, J=7.2, 0.8 Hz, 1H), 6.59-6.48 (m, 1H), 5.04 (s, 1H), 3.55 (dd, J=14.7, 6.3 Hz, 2H), 2.91 (t, J=7.5 Hz, 2H); MS (ESI) m/z: 372.1 [M+H⁺].

N-(3-Chlorobenzyl)-2-ethynyl-5-methylthiazole-4-carboxamide C52. ¹H NMR (400 MHz, DMSO-d₆) δ 9.07 (t, J=6.3 Hz, 1H), 7.38-7.24 (m, 4H), 4.98 (s, 1H), 4.40 (d, J=6.4 Hz, 2H), 2.75 (s, 3H); MS (ESI) m/z: 291.1 [M+H⁺].

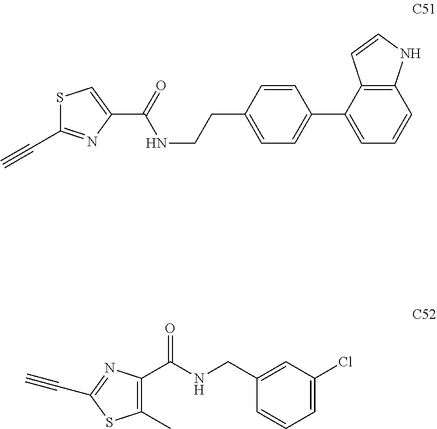 [SMILES: C#Cc1nc(C(=O)NCCc2ccc(-c3cccc4[nH]ccc34)cc2)cs1 ; C#Cc1nc(C(=O)NCc2cccc(Cl)c2)c(C)s1]

2-Ethynyl-N-(4-(thiazol-4-yl)phenethyl)thiazole-4-carboxamide C53. ¹H NMR (400 MHz, DMSO-d₆) δ 9.18 (d, J=1.9 Hz, 1H), 8.75-8.50 (m, 1H), 8.35 (s, 1H), 8.11 (d, J=1.6 Hz, 1H), 7.91 (d, J=8.2 Hz, 2H), 7.31 (d, J=8.2 Hz, 2H), 5.04 (s, 1H), 3.52 (dd, J=14.0, 6.7 Hz, 2H), 2.88 (t, J=7.4 Hz, 2H); MS (ESI) m/z: 340.0 [M+H⁺].

2-Ethynyl-N-(4-(1-methyl-1H-pyrazol-3-yl)benzyl)thiazole-4-carboxamide C54. ¹H NMR (400 MHz, DMSO-d₆) δ 9.14 (t, J=6.3 Hz, 1H), 8.40 (s, 1H), 7.71 (t, J=5.0 Hz, 3H), 7.32 (d, J=8.3 Hz, 2H), 6.64 (d, J=2.3 Hz, 1H), 5.05 (s, 1H), 4.44 (d, J=6.3 Hz, 2H), 3.87 (s, 3H); MS (ESI) m/z: 323.1 [M+H⁺].

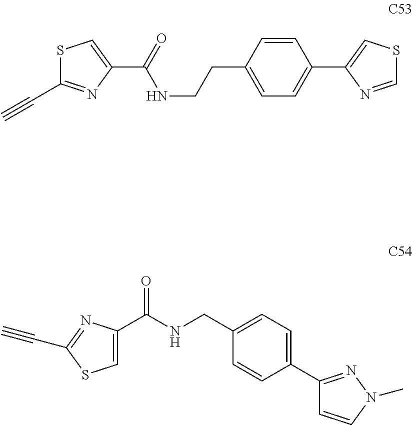 [SMILES: C#Cc1nc(C(=O)NCCc2ccc(-c3cscn3)cc2)cs1 ; C#Cc1nc(C(=O)NCc2ccc(-c3ccn(C)n3)cc2)cs1]

2-Ethynyl-N-(4-(thiazol-4-yl)benzyl)thiazole-4-carboxamide C55. ¹H NMR (400 MHz, DMSO-d₆) δ 9.18 (d, J=1.9 Hz, 2H), 8.40 (s, 1H), 8.12 (d, J=1.9 Hz, 1H), 7.93 (d, J=8.3 Hz, 2H), 7.39 (d, J=8.3 Hz, 2H), 5.05 (s, 1H), 4.47 (d, J=6.3 Hz, 2H); MS (ESI) m/z: 326.0 [M+H⁺].

N-(3-Chlorobenzyl)-2-ethynyl-5-phenylthiazole-4-carboxamide C56. ¹H NMR (400 MHz, DMSO-d₆) δ 9.18 (t, J=6.2 Hz, 1H), 7.60-7.50 (m, 2H), 7.46-7.38 (m, 3H), 7.37-7.28 (m, 3H), 7.24 (d, J=7.4 Hz, 1H), 5.11 (s, 1H), 4.38 (d, J=6.3 Hz, 2H); MS (ESI) m/z: 352.2 [M+H⁺].

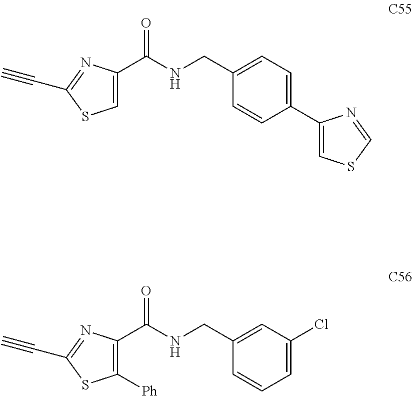 [SMILES: C#Cc1nc(C(=O)NCc2ccc(-c3cscn3)cc2)cs1 ; C#Cc1nc(C(=O)NCc2cccc(Cl)c2)c(-c2ccccc2)s1]

2-Ethynyl-N-(4-(thiazol-5-yl)phenethyl)thiazole-4-carboxamide C57. ¹H NMR (400 MHz, DMSO-d₆) δ 9.06 (d, J=0.4 Hz, 1H), 8.65 (t, J=5.9 Hz, 1H), 8.35 (s, 1H), 8.28 (s, 1H), 7.61 (d, J=8.1 Hz, 2H), 7.31 (d, J=8.2 Hz, 2H), 5.04 (s, 1H), 3.52 (dd, J=14.0, 6.6 Hz, 3H), 2.88 (t, J=7.3 Hz, 2H); MS (ESI) m/z: 340.0 [M+H⁺].

N-(3-(1H-Pyrazol-4-yl)benzyl)-2-ethynylthiazole-4-carboxamide C58. ¹H NMR (400 MHz, DMSO-d₆) δ 12.92 (s, 1H), 9.11 (t, J=6.3 Hz, 1H), 8.39 (s, 1H), 8.14 (s, 1H), 7.87 (s, 1H), 7.54 (s, 1H), 7.47 (d, J=7.8 Hz, 1H), 7.29 (t, J=7.6 Hz, 1H), 7.13 (d, J=7.7 Hz, 1H), 5.04 (s, 1H), 4.45 (d, J=6.3 Hz, 2H); MS (ESI) m/z: 309.1 [M+H⁺].

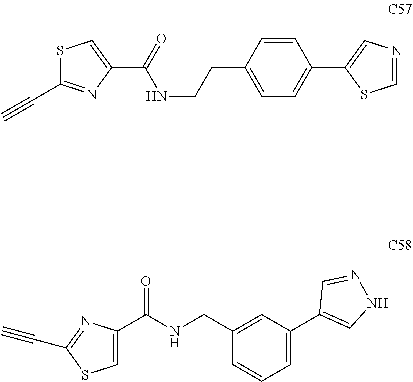 [SMILES: C#Cc1nc(C(=O)NCCc2ccc(-c3cncs3)cc2)cs1 ; C#Cc1nc(C(=O)NCc2cccc(-c3cn[nH]c3)c2)cs1]

2-Ethynyl-N-(3-(1-methyl-1H-pyrazol-4-yl)benzyl)thiazole-4-carboxamide C59. ¹H NMR (400 MHz, DMSO-d₆) δ 9.12 (t, J=6.3 Hz, 1H), 8.39 (s, 1H), 8.08 (s, 1H), 7.80 (d, J=0.7 Hz, 1H), 7.56-7.47 (m, 1H), 7.42 (d, J=7.8 Hz, 1H), 7.29 (t, J=7.6 Hz, 1H), 7.13 (d, J=7.7 Hz, 1H), 5.05 (s, 1H), 4.45 (d, J=6.3 Hz, 2H), 3.86 (s, 3H); MS (ESI) m/z: 323.0 [M+H⁺].

N-(3-(1H-Pyrazol-3-yl)benzyl)-2-ethynylthiazole-4-carboxamide C60. ¹H NMR (400 MHz, DMSO-d₆) δ 12.85 (s, 1H), 9.17 (t, J=6.2 Hz, 1H), 8.39 (d, J=4.4 Hz, 1H), 7.91-7.50 (m, 3H), 7.46-7.13 (m, 2H), 6.65 (d, J=2.1 Hz, 1H), 5.05 (s, 1H), 4.48 (d, J=6.3 Hz, 2H); MS (ESI) m/z: 309.0 [M+H⁺].

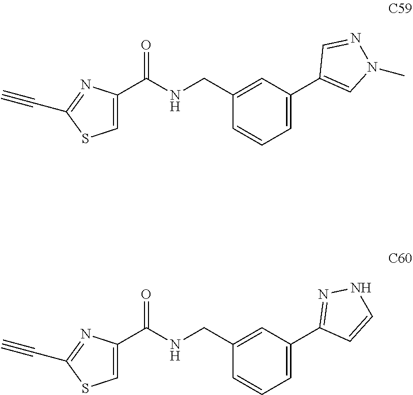

2-Ethynyl-N-(4-(thiazol-5-yl)benzyl)thiazole-4-carboxamide C61. ¹H NMR (400 MHz, DMSO-d₆) δ 9.19 (t, J=6.3 Hz, 1H), 9.06 (d, J=0.5 Hz, 1H), 8.40 (s, 1H), 8.27 (d, J=0.5 Hz, 1H), 7.64 (d, J=8.3 Hz, 2H), 7.39 (d, J=8.3 Hz, 2H), 5.05 (s, 1H), 4.46 (d, J=6.3 Hz, 2H); MS (ESI) m/z: 326.0 [M+H⁺].

N-(2,3-Dihydro-1H-inden-2-yl)-2-ethynylthiazole-4-carboxamide C62. ¹H NMR (400 MHz, DMSO-d₆) δ 8.71 (d, J=4.0 Hz, 1H), 8.39 (s, 1H), 7.22-7.19 (m, 2H), 7.17-7.13 (m, 2H), 5.03 (s, 1H), 4.73-4.65 (m, 1H), 3.17 (dd, J=16.0, 8.0 Hz, 2H), 3.03 (dd, J=16.0, 8.0 Hz, 2H); MS (ESI) m/z: 269.2 [M+H⁺].

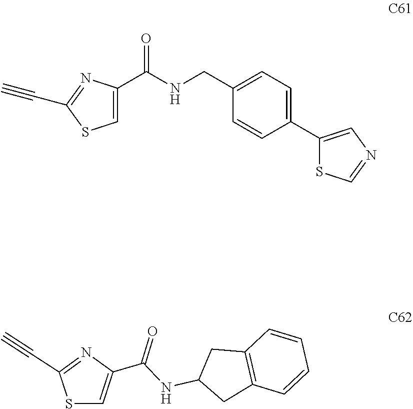 [SMILES: C#Cc1nc(C(=O)NCc2ccc(-c3cncs3)cc2)cs1 ; C#Cc1nc(C(=O)NC2Cc3ccccc3C2)cs1]

N-(2-Cyanobenzyl)-2-ethynylthiazole-4-carboxamide C63. ¹H NMR (400 MHz, DMSO-d₆) δ 9.25 (t, J=6.0 Hz, 1H), 8.41 (s, 1H), 7.74-7.71 (m, 2H), 7.66 (d, J=8.0 Hz, 1H), 7.54 (t, J=8.0 Hz, 2H), 5.06 (s, 1H), 4.49 (d, J=8.0 Hz, 2H); MS (ESI) m/z: 268.1 [M+H⁺].

2-Ethynyl-N-(naphthalen-2-ylmethyl)thiazole-4-carboxamide C64. ¹H NMR (400 MHz, DMSO-d₆) δ 9.24 (t, J=6.3 Hz, 1H), 8.41 (s, 1H), 7.89-7.86 (m, 3H), 7.78 (s, 1H), 7.51-7.47 (m, 3H), 5.05 (s, 1H), 4.61 (d, J=6.3 Hz, 2H); MS (ESI) m/z: 293.0 [M+H⁺].

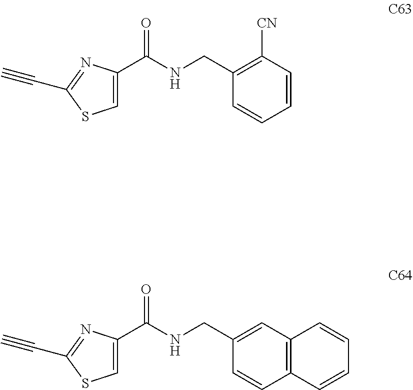 [SMILES: C#Cc1nc(C(=O)NCc2ccccc2C#N)cs1 ; C#Cc1nc(C(=O)NCc2ccc3ccccc3c2)cs1]

2-Ethynyl-N-(3-(pyridin-3-yl)benzyl)thiazole-4-carboxamide C65. ¹H NMR (400 MHz, DMSO-d₆) δ 9.20 (t, J=6.3 Hz, 1H), 8.99 (d, J=1.6 Hz, 1H), 8.69 (d, J=4.4 Hz, 1H), 8.40 (s, 1H), 8.33 (d, J=7.8 Hz, 1H), 7.72 (d, J=9.2 Hz, 2H), 7.66 (d, J=7.7 Hz, 1H), 7.49 (t, J=7.6 Hz, 1H), 7.42 (d, J=7.7 Hz, 1H), 5.05 (s, 1H), 4.54 (d, J=6.3 Hz, 2H); MS (ESI) m/z: 320.1 [M+H⁺].

N-(Benzo[d]thiazol-6-ylmethyl)-2-ethynylthiazole-4-carboxamide C66. ¹H NMR (400 MHz, DMSO-d₆) δ 9.34 (s, 1H), 9.25 (t, J=6.3 Hz, 1H), 8.40 (s, 1H), 8.05 (dd, J=13.8, 4.8 Hz, 2H), 7.51 (dd, J=8.4, 1.7 Hz, 1H), 5.05 (s, 1H), 4.59 (d, J=6.3 Hz, 2H); MS (ESI) m/z: 300.1 [M+H⁺].

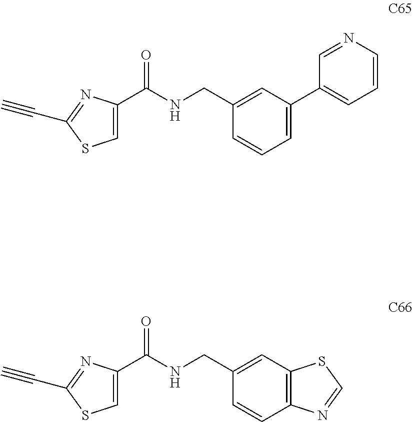 [SMILES: C#Cc1nc(C(=O)NCc2cccc(-c3cccnc3)c2)cs1 ; C#Cc1nc(C(=O)NCc2ccc3ncsc3c2)cs1]

(R)—N-(1-(3-Chlorophenyl)ethyl)-2-ethynylthiazole-4-carboxamide C67. ¹H NMR (400 MHz, DMSO-d₆) δ 8.99 (d, J=8.4 Hz, 1H), 8.36 (s, 1H), 7.49 (d, J=1.6 Hz, 1H), 7.37-7.27 (m, 3H), 5.14 (p, 1H), 5.06 (s, 1H), 1.49 (d, J=7.1 Hz, 3H); MS (ESI) m/z: 291.0 [M+H⁺].

N-(1-(3-Chlorophenyl)cyclopropyl)-2-ethynylthiazole-4-carboxamide C68. ¹H NMR (400 MHz, DMSO-d₆) δ 9.34 (s, 1H), 8.38 (s, 1H), 7.30 (t, J=7.8 Hz, 1H), 7.25-7.21 (m, 2H), 7.18-7.14 (m, 1H), 5.05 (s, 1H), 1.31-1.26 (m, 4H); MS (ESI) m/z: 303.0 [M+H⁺].

(S)—N-(1-(3-Chlorophenyl)ethyl)-2-ethynylthiazole-4-carboxamide C69. ¹H NMR (400 MHz, DMSO-d₆) δ 8.99 (d, J=8.4 Hz, 1H), 8.36 (s, 1H), 7.50-7.48 (m, 1H), 7.37-7.32 (m, 2H), 7.30-7.27 (m, 1H), 5.14 (p, 1H), 5.06 (s, 1H), 1.49 (d, J=7.1 Hz, 3H); MS (ESI) m/z: 291.0 [M+H⁺].

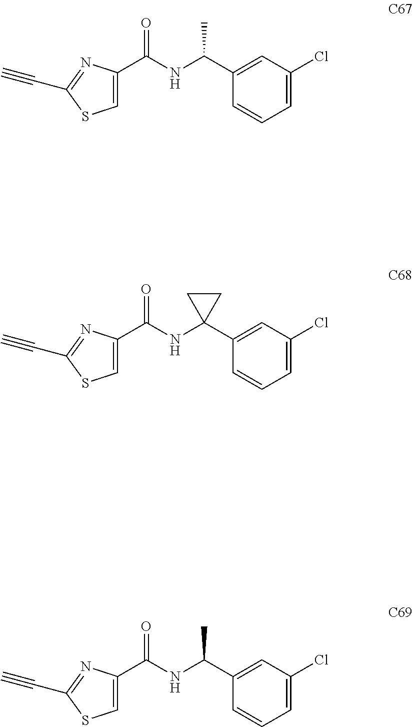 [SMILES: C#Cc1nc(C(=O)N[C@H](C)c2cccc(Cl)c2)cs1 ; C#Cc1nc(C(=O)NC2(c3cccc(Cl)c3)CC2)cs1 ; C#Cc1nc(C(=O)N[C@@H](C)c2cccc(Cl)c2)cs1]

2-Ethynyl-N-(2-(hydroxymethyl)benzyl)thiazole-4-carboxamide C70. ¹H NMR (400 MHz, DMSO-d₆) δ 8.97 (t, J=6.1 Hz, 1H), 8.39 (s, 1H), 7.39-7.35 (m, 1H), 7.28 (dd, J=6.2, 2.8 Hz, 1H), 7.24-7.20 (m, 2H), 5.04 (s, 1H), 4.61 (s, 2H), 4.49 (d, J=6.2 Hz, 2H), 4.08 (s, 1H); MS (ESI) m/z: 273.1 [M+H⁺].

(S)—N-(2,3-Dihydro-1H-inden-1-yl)-2-ethynylthiazole-4-carboxamide C71. ¹H NMR (400 MHz, DMSO-d₆) δ 8.75 (d, J=8.6 Hz, 1H), 8.44 (s, 1H), 7.25-7.18 (m, 4H), 5.52 (d, J=8.2 Hz, 1H), 5.02 (s, 1H), 3.02-2.97 (m, 1H), 2.87-2.79 (m, 1H), 2.43-2.37 (m, 1H), 2.14-2.07 (m, 1H); MS (ESI) m/z: 269.0 [M+H⁺].

(R)—N-(2,3-Dihydro-1H-inden-1-yl)-2-ethynylthiazole-4-carboxamide C72. ¹H NMR (400 MHz, DMSO-d₆) δ 8.75 (d, J=8.6 Hz, 1H), 8.44 (s, 1H), 7.25-7.18 (m, 4H), 5.52 (q, J=8.1 Hz, 1H), 5.02 (s, 1H), 3.02-2.97 (m, 1H), 2.87-2.80 (m, 1H), 2.42-2.37 (m, 1H), 2.13-2.07 (m, 1H); MS (ESI) m/z: 269.0 [M+H⁺].

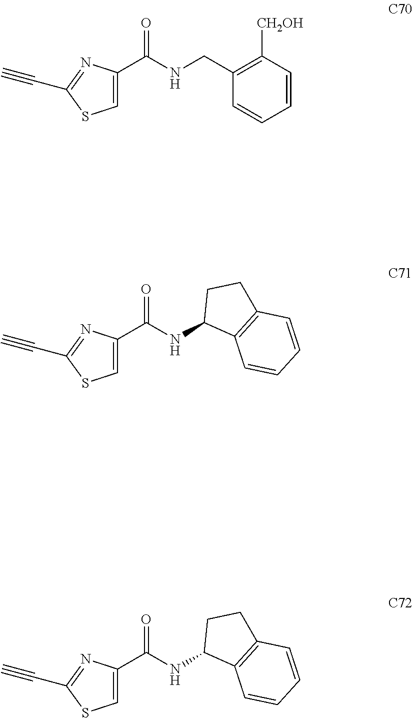 [SMILES: C#Cc1nc(C(=O)NCc2ccccc2CO)cs1 ; C#Cc1nc(C(=O)N[C@H]2CCc3ccccc32)cs1 ; C#Cc1nc(C(=O)N[C@@H]2CCc3ccccc32)cs1]

Methyl 3-(4-((2-ethynylthiazole-4-carboxamido)methyl)phenyl)propanoate C73. ¹H NMR (400 MHz, DMSO-d₆) δ 9.09 (t, J=6.3 Hz, 1H), 8.38 (s, 1H), 7.22 (d, J=8.1 Hz, 2H), 7.15 (d, J=8.1 Hz, 2H), 5.04 (s, 1H), 4.39 (d, J=6.3 Hz, 2H), 3.57 (s, 3H), 2.81 (s, 2H), 2.61 (d, J=7.6 Hz, 2H); MS (ESI) m/z: 329.1 [M+H⁺].

Methyl 2-((2-ethynylthiazole-4-carboxamido)methyl)benzoate C74. ¹H NMR (400 MHz, DMSO-d₆) δ 8.99 (t, J=6.0 Hz, 1H), 8.41 (s, 1H), 7.88 (d, J=8.0 Hz, 1H), 7.59-7.55 (m, 1H), 7.43-7.37 (m, 2H), 5.07 (s, 1H), 4.76 (d, J=4.0 Hz, 2H), 3.87 (s, 3H); MS (ESI) m/z: 301.1 [M+H⁺].

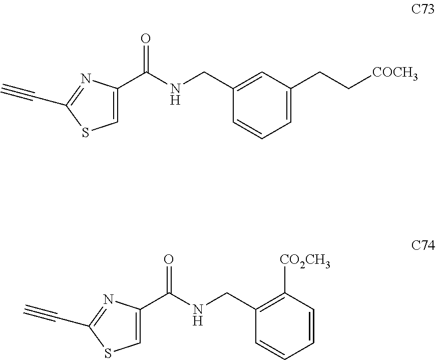 [SMILES: C#Cc1nc(C(=O)NCc2cccc(CCC(C)=O)c2)cs1 ; C#Cc1nc(C(=O)NCc2ccccc2C(=O)OC)cs1]

2-Ethynyl-N-(4-(1-methyl-1H-pyrazol-4-yl)phenethyl)thiazole-4-carboxamide C75. ¹H NMR (400 MHz, DMSO-d₆) δ 8.62 (t, J=5.9 Hz, 1H), 8.35 (s, 1H), 8.08 (s, 1H), 7.81 (d, J=0.6 Hz, 1H), 7.47 (d, J=8.2 Hz, 2H), 7.20 (d, J=8.2 Hz, 2H), 5.04 (s, 1H), 3.85 (s, 3H), 3.49 (dd, J=14.3, 6.4 Hz, 2H), 2.83 (t, J=7.4 Hz, 2H); MS (ESI) m/z: 337.1 [M+H⁺].

N-(4-(1H-Pyrazol-4-yl)phenethyl)-2-ethynylthiazole-4-carboxamide C76. ¹H NMR (400 MHz, DMSO-d₆) δ 12.88 (s, 1H), 8.62 (t, J=5.9 Hz, 1H), 8.35 (s, 1H), 8.13 (s, 1H), 7.88 (s, 1H), 7.52 (d, J=8.2 Hz, 2H), 7.20 (d, J=8.2 Hz, 2H), 5.04 (s, 1H), 3.49 (dd, J=14.3, 6.5 Hz, 2H), 2.83 (t, J=7.4 Hz, 2H); MS (ESI) m/z: 323.0 [M+H⁺].

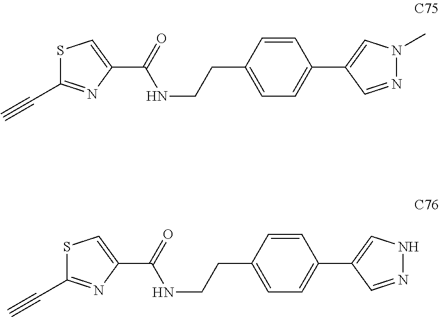

N-(2-Ethynylthiazol-4-yl)-2-phenylacetamide C77. ¹H NMR (400 MHz, DMSO-d₆) δ 11.35 (s, 1H), 7.67 (s, 1H), 7.36-7.29 (m, 4H), 7.26 (dd, J=4.9, 3.8 Hz, 1H), 4.94 (s, 1H), 3.68 (s, 2H); MS (ESI) m/z: 243.1 [M+H⁺].

2-Ethynyl-N-(1-phenylpiperidin-4-yl)thiazole-4-carboxamide C78. ¹H NMR (400 MHz, DMSO-d₆) δ 9.89 (s, 1H), 9.11 (t, J=6.0 Hz, 1H), 8.39 (s, 1H), 7.27-6.87 (m, 5H), 5.05 (s, 1H), 4.01 (s, 1H), 3.71 (d, J=16.0 Hz, 1H), 1.86 (s, 5H); MS (ESI) m/z: 312.4 [M+H⁺].

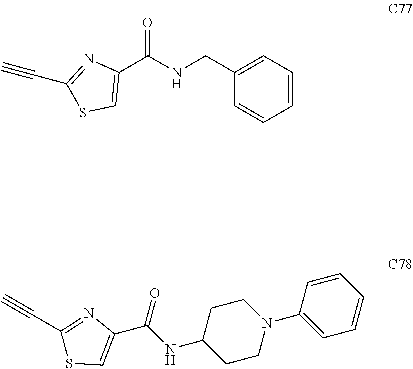 [SMILES: C#Cc1nc(C(=O)NCc2ccccc2)cs1 ; C#Cc1nc(C(=O)NC2CCN(c3ccccc3)CC2)cs1]

2-Ethynyl-N-(4-(pyridin-3-yl)phenethyl)thiazole-4-carboxamide C79. ¹H NMR (400 MHz, DMSO-d₆) δ 8.88 (d, J=1.7 Hz, 1H), 8.67 (t, J=5.9 Hz, 1H), 8.55 (dd, J=4.7, 1.6 Hz, 1H), 8.36 (s, 1H), 8.10-8.02 (m, 1H), 7.67 (d, J=8.2 Hz, 2H), 7.47 (ddd, J=8.0, 4.8, 0.7 Hz, 1H), 7.37 (d, J=8.2 Hz, 2H), 5.05 (s, 1H), 3.54 (dd, J=14.2, 6.5 Hz, 2H), 2.91 (t, J=7.4 Hz, 2H); MS (ESI) m/z: 334.0 [M+H⁺].

N-(4-Bromophenethyl)-2-ethynylthiazole-4-carboxamide C80. ¹H NMR (400 MHz, DMSO-d₆) δ 8.63 (t, J=5.8 Hz, 1H), 8.34 (s, 1H), 7.52-7.38 (m, 2H), 7.19 (d, J=8.4 Hz, 2H), 5.05 (s, 1H), 3.48 (dd, J=13.9, 6.6 Hz, 2H), 2.82 (t, J=7.3 Hz, 2H); MS (ESI) m/z: 335.0 & 336.9 [M+H⁺].

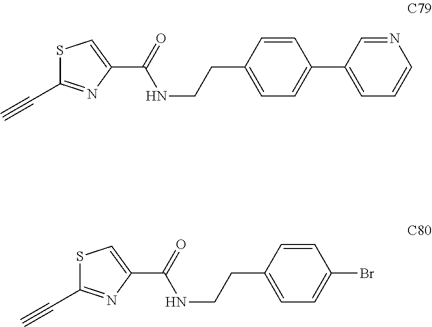 [SMILES: C#Cc1nc(C(=O)NCCc2ccc(-c3cccnc3)cc2)cs1 ; C#Cc1nc(C(=O)NCCc2ccc(Br)cc2)cs1]

2-Ethynyl-N-(3-(methylsulfonamido)phenethyl)thiazole-4-carboxamide C81. ¹H NMR (400 MHz, DMSO-d₆) δ 9.66 (s, 1H), 8.63 (t, J=5.9 Hz, 1H), 8.34 (s, 1H), 7.25 (t, J=7.7 Hz, 1H), 7.06 (t, J=5.3 Hz, 2H), 6.98 (d, J=7.6 Hz, 1H), 5.04 (s, 1H), 3.47 (dd, J=14.4, 6.4 Hz, 2H), 2.94 (s, 3H), 2.81 (t, J=7.4 Hz, 2H); MS (ESI) m/z: 350.0 [M+H⁺].

N-(3-Acetamidophenethyl)-2-ethynylthiazole-4-carboxamide C82. ¹H NMR (400 MHz, DMSO-d₆) δ 9.88 (s, 1H), 8.65 (t, J=5.8 Hz, 1H), 8.36 (s, 1H), 7.50-7.39 (m, 2H), 7.20 (dd, J=8.5, 7.7 Hz, 1H), 6.89 (d, J=7.5 Hz, 1H), 5.05 (s, 1H), 3.45 (dd, J=15.1, 6.2 Hz, 2H), 2.85-2.73 (m, 2H), 2.03 (s, 3H); MS (ESI) m/z: 314.0 [M+H⁺].

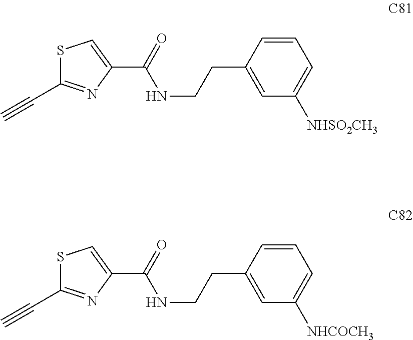

2-Ethynyl-N-(4-(1-methyl-1H-pyrazol-4-yl)benzyl)thiazole-4-carboxamide C83. ¹H NMR (400 MHz, DMSO-d₆) δ 8.67 (t, J=5.9 Hz, 1H), 8.34 (s, 1H), 7.89 (d, J=8.2 Hz, 2H), 7.38 (d, J=8.2 Hz, 2H), 5.05 (s, 1H), 3.84 (s, 3H), 3.62-3.44 (m, 2H), 2.93 (t, J=7.3 Hz, 2H); MS (ESI) m/z: 323.1 [M+H⁺].

Methyl 4-(2-(2-ethynylthiazole-4-carboxamido)ethyl)benzoate C84. ¹H NMR (400 MHz, DMSO-d₆) δ 8.67 (t, J=5.9 Hz, 1H), 8.34 (s, 1H), 7.89 (d, J=8.2 Hz, 2H), 7.38 (d, J=8.2 Hz, 2H), 5.05 (s, 1H), 3.84 (s, 3H), 3.62-3.44 (m, 2H), 2.93 (t, J=7.3 Hz, 2H); MS (ESI) m/z: 315.1 [M+H⁺].

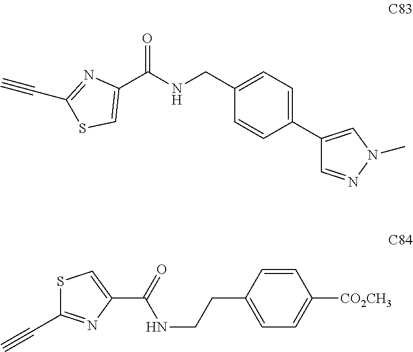

N-(3-Aminophenethyl)-2-ethynylthiazole-4-carboxamide C85. ¹H NMR (400 MHz, DMSO-d₆) δ 8.59 (t, J=5.9 Hz, 1H), 8.36 (s, 1H), 6.92 (t, J=7.7 Hz, 1H), 6.43 (d, J=1.6 Hz, 1H), 6.42-6.35 (m, 2H), 5.05 (s, 1H), 4.98 (s, 2H), 3.42 (dt, J=7.7, 6.1 Hz, 2H), 2.70-2.63 (m, 2H); MS (ESI) m/z: 272.0 [M+H⁺].

N-((2,3-Dihydrobenzo[b][1,4]dioxin-6-yl)methyl)-2-ethynylthiazole-4-carboxamide C86. ¹H NMR (400 MHz, DMSO-d₆) δ 9.04 (t, J=6.3 Hz, 1H), 8.38 (s, 1H), 6.79 (t, J=8.2 Hz, 3H), 5.05 (s, 1H), 4.30 (d, J=6.3 Hz, 2H), 4.20 (s, 4H); MS (ESI) m/z: 301.1 [M+H⁺].

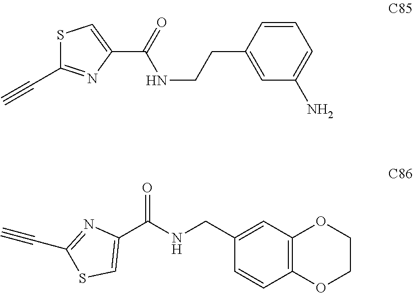 [SMILES: C#Cc1nc(C(=O)NCCc2cccc(N)c2)cs1 ; C#Cc1nc(C(=O)NCc2ccc3c(c2)OCCO3)cs1]

2-Ethynyl-N-(3-nitrophenethyl)thiazole-4-carboxamide C87. ¹H NMR (400 MHz, DMSO-d₆) δ 8.69 (t, J=5.9 Hz, 1H), 8.33 (s, 1H), 8.13-8.03 (m, 2H), 7.70 (d, J=7.7 Hz, 1H), 7.59 (t, J=7.9 Hz, 1H), 5.04 (s, 1H), 3.56 (dd, J=13.2, 6.9 Hz, 2H), 3.01 (t, J=7.0 Hz, 2H); MS (ESI) m/z: 302.1 [M+H⁺].

N-(4-Acetamidophenethyl)-2-ethynylthiazole-4-carboxamide C88. ¹H NMR (400 MHz, DMSO-d₆) δ 9.85 (s, 1H), 8.59 (t, J=5.9 Hz, 1H), 8.34 (s, 1H), 7.48 (d, J=8.4 Hz, 2H), 7.13 (d, J=8.4 Hz, 2H), 5.04 (s, 1H), 3.45 (q, J=14.5, 6.4 Hz, 2H), 2.77 (t, J=7.5 Hz, 2H), 2.01 (s, 3H); MS (ESI) m/z: 314.1 [M+H⁺].

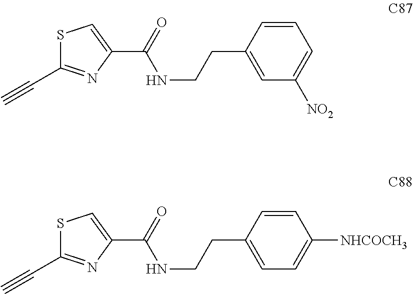

N-(4-Acetamidobenzyl)-2-ethynylthiazole-4-carboxamide C89. ¹H NMR (400 MHz, DMSO-d₆) δ 9.88 (s, 1H), 9.07 (d, J=6.2 Hz, 1H), 8.38 (s, 1H), 7.49 (d, J=8.5 Hz, 2H), 7.22 (d, J=8.5 Hz, 2H), 5.04 (s, 1H), 4.37 (d, J=6.3 Hz, 2H), 3.32 (s, 3H); MS (ESI) m/z: 300.1 [M+H⁺].

N-Benzyl-2-cyanothiazole-4-carboxamide C90. ¹H NMR (400 MHz, DMSO-d₆) δ 9.34 (t, J=6.2 Hz, 1H), 8.75 (s, 1H), 7.31 (d, J=4.4 Hz, 4H), 7.25-7.22 (m, 1H), 4.45 (d, J=6.3 Hz, 2H); MS (ESI) m/z: 244.1 [M+H⁺].

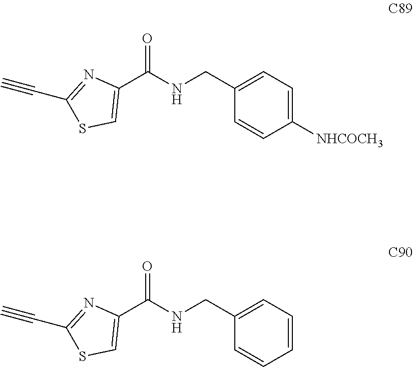

N-(4-(1H-Pyrazol-3-yl)benzyl)-2-ethynylthiazole-4-carboxamide C91. ¹H NMR (400 MHz, DMSO-d₆) δ 8.19 (s, 1H), 7.72 (d, J=7.8 Hz, 4H), 7.41 (d, J=7.5 Hz, 2H), 6.66 (s, 1H), 4.67 (d, J=5.6 Hz, 2H), 3.52 (s, 1H); MS (ESI) m/z: 309.0 [M+H⁺].

N-(4-(1H-Pyrazol-4-yl)benzyl)-2-ethynylthiazole-4-carboxamide C92. ¹H NMR (400 MHz, DMSO-d₆) δ 9.10 (d, J=6.3 Hz, 1H), 8.39 (s, 1H), 8.02 (s, 2H), 7.59-7.49 (m, 2H), 7.29 (d, J=8.3 Hz, 2H), 5.05 (s, 1H), 4.42 (d, J=6.3 Hz, 2H); MS (ESI) m/z: 309.1 [M+H⁺].

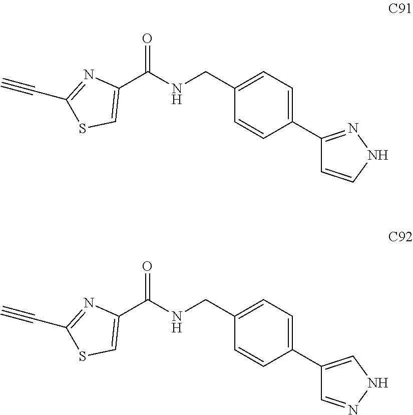 [SMILES: C#Cc1nc(C(=O)NCc2ccc(-c3cc[nH]n3)cc2)cs1 ; C#Cc1nc(C(=O)NCc2ccc(-c3cn[nH]c3)cc2)cs1]

2-Ethynyl-N-(4-(pyridin-3-yl)benzyl)thiazole-4-carboxamide C93. ¹H NMR (400 MHz, DMSO-d₆) δ 9.21 (t, J=6.3 Hz, 1H), 8.91 (d, J=1.9 Hz, 1H), 8.59 (dd, J=4.8, 1.3 Hz, 1H), 8.40 (s, 1H), 8.13 (d, J=6.7 Hz, 1H), 7.69 (d, J=8.3 Hz, 2H), 7.54 (dd, J=7.9, 4.9 Hz, 1H), 7.45 (d, J=8.3 Hz, 2H), 5.06 (s, 1H), 4.49 (d, J=6.3 Hz, 2H); MS (ESI) m/z: 320.1 [M+H⁺].

N-(3-Acetamidobenzyl)-2-ethynylthiazole-4-carboxamide C94. ¹H NMR (400 MHz, DMSO-d₆) δ 9.89 (s, 1H), 9.11 (t, J=6.0 Hz, 1H), 8.39 (s, 1H), 7.52 (d, J=8.0 Hz, 1H), 7.43 (s, 1H), 7.21 (t, J=8.0 Hz, 1H), 6.97 (d, J=8.0 Hz, 1H), 5.05 (s, 1H), 4.40 (d, J=8.0 Hz, 2H) 2.01 (s, 3H); MS (ESI) m/z: 300.4 [M+H⁺].

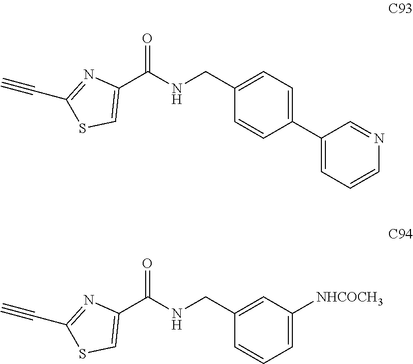

N-(3-Aminobenzyl)-2-ethynylthiazole-4-carboxamide C95. ¹H NMR (400 MHz, DMSO-d₆) δ 8.95 (t, J=6.0 Hz, 1H), 8.38 (s, 1H), 8.93 (t, J=8.0 Hz, 1H), 6.49 (d, J=4.0 Hz, 1H), 6.44-6.40 (m, 2H), 5.06-4.98 (m, 3H), 4.30 (d, J=4.0 Hz, 2H); MS (ESI) m/z: 258.1 [M+H⁺].

2-Ethynyl-N-(quinolin-6-ylmethyl)thiazole-4-carboxamide C96. ¹H NMR (400 MHz, DMSO-d₆) δ 9.34 (s, 1H), 8.99 (d, J=10.0 Hz, 1H), 8.62 (d, J=23.3 Hz, 1H), 8.42 (s, 1H), 8.05 (t, J=8.3 Hz, 1H), 7.96 (d, J=12.1 Hz, 1H), 7.86 (t, J=10.5 Hz, 1H), 7.70 (s, 1H), 5.06 (s, 1H), 4.66 (d, J=6.1 Hz, 2H); MS (ESI) m/z: 294.1 [M+H⁺].

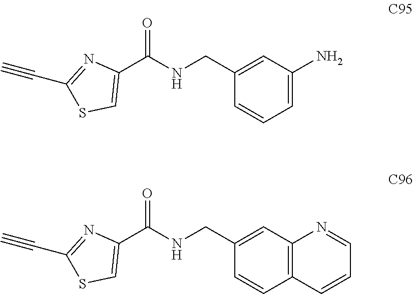 [SMILES: C#Cc1nc(C(=O)NCc2cccc(N)c2)cs1 ; C#Cc1nc(C(=O)NCc2ccc3cccnc3c2)cs1]

Methyl 3-(2-(2-ethynylthiazole-4-carboxamido)ethyl)benzoate C97. ¹H NMR (400 MHz, DMSO-d₆) δ 8.65 (t, J=5.9 Hz, 1H), 8.33 (s, 1H), 7.86-7.77 (m, 2H), 7.51 (d, J=7.7 Hz, 1H), 7.44 (t, J=7.6 Hz, 1H), 5.04 (s, 1H), 3.84 (s, 3H), 3.50 (dd, J=14.1, 6.5 Hz, 2H), 2.92 (t, J=7.3 Hz, 2H); MS (ESI) m/z: 315.1 [M+H⁺].

N-Benzyl-2-ethynyl-1-methyl-1H-imidazole-4-carboxamide C98. ¹H NMR (400 MHz, DMSO-d₆) δ 8.62 (t, J=6.4 Hz, 1H), 7.78 (s, 1H), 7.29 (d, J=6.6 Hz, 4H), 7.24-7.18 (m, 1H), 4.72 (s, 1H), 4.38 (d, J=6.4 Hz, 2H), 3.72 (s, 3H); MS (ESI) m/z: 240.1 [M+H⁺].

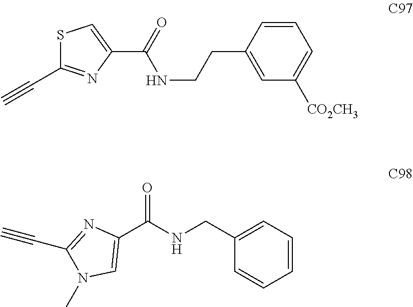 [SMILES: C#Cc1nc(C(=O)NCCc2cccc(C(=O)OC)c2)cs1 ; C#Cc1nc(C(=O)NCc2ccccc2)cn1C]

2-Ethynyl-N-(4-(methylsulfonamido)phenethyl)thiazole-4-carboxamide C99. ¹H NMR (400 MHz, DMSO-d₆) δ 9.60 (s, 1H), 8.61 (s, 1H), 8.34 (s, 1H), 7.20-7.17 (m, 2H), 7.14-7.11 (m, 2H), 5.04 (s, 1H), 3.46 (q, J=14.6, 6.3 Hz, 2H), 2.94 (s, 3H), 2.79 (t, J=7.4 Hz, 2H); MS (ESI) m/z: 350.1 [M+H⁺].

2-Ethynyl-N-methyl-N-(4-nitrophenethyl)thiazole-4-carboxamide C100. ¹H NMR (400 MHz, DMSO-d₆) δ 8.19-8.15 (m, 1H), 8.12-7.89 (m, 2H), 7.59-7.41 (m, 2H), 5.07-4.99 (m, 1H), 3.80-3.69 (m, 2H), 3.07-3.02 (m, 5H); MS (ESI) m/z: 316.0 [M+H⁺].

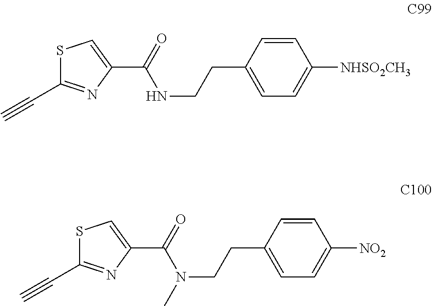

2-Ethynyl-N-(4-(methylsulfonyl)phenethyl)thiazole-4-carboxamide C101. ¹H NMR (400 MHz, DMSO-d₆) δ 8.69 (t, J=5.8 Hz, 1H), 8.35 (s, 1H), 7.84 (d, J=8.3 Hz, 2H), 7.50 (d, J=8.3 Hz, 2H), 5.04 (s, 1H), 3.53 (dd, J=13.5, 6.9 Hz, 2H), 3.18 (s, 3H), 2.97 (t, J=7.2 Hz, 2H); MS (ESI) m/z: 335.0 [M+H⁺].

N-(4-Cyanophenethyl)-2-ethynylthiazole-4-carboxamide C102. ¹H NMR (400 MHz, DMSO-d₆) δ 8.66 (t, J=5.8 Hz, 1H), 8.34 (s, 1H), 7.75 (d, J=8.3 Hz, 2H), 7.43 (d, J=8.3 Hz, 2H), 5.04 (s, 1H), 3.52 (dd, J=13.3, 6.9 Hz, 2H), 2.94 (t, J=7.1 Hz, 2H); MS (ESI) m/z: 282.0 [M+H⁺].

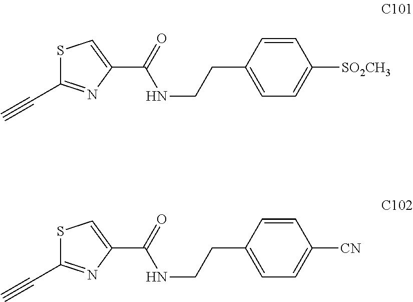 [SMILES: C#Cc1nc(C(=O)NCCc2ccc(S(C)(=O)=O)cc2)cs1 ; C#Cc1nc(C(=O)NCCc2ccc(C#N)cc2)cs1]

2-Ethynyl-N-(4-nitrobenzyl)thiazole-4-carboxamide C103. ¹H NMR (400 MHz, DMSO-d₆) δ 9.33 (t, J=6.2 Hz, 1H), 8.42 (s, 1H), 8.19 (d, J=8.8 Hz, 2H), 7.57 (d, J=8.8 Hz, 2H), 5.06 (s, 1H), 4.56 (d, J=6.3 Hz, 2H); MS (ESI) m/z: 288.1 [M+H⁺].

N-(4-Cyanobenzyl)-2-ethynylthiazole-4-carboxamide C104. ¹H NMR (400 MHz, DMSO-d₆) δ 9.28 (t, J=6.3 Hz, 1H), 8.41 (s, 1H), 7.80-7.77 (m, 2H), 7.49 (d, J=8.4 Hz, 2H), 5.06 (s, 1H), 4.51 (d, J=6.3 Hz, 2H); MS (ESI) m/z: 268.0 [M+H⁺].

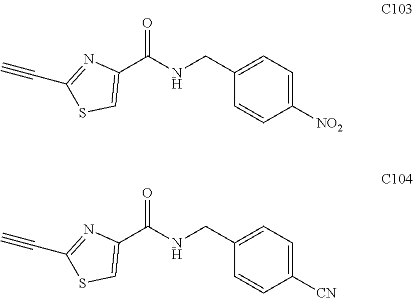 [SMILES: C#Cc1nc(C(=O)NCc2ccc([N+](=O)[O-])cc2)cs1 ; C#Cc1nc(C(=O)NCc2ccc(C#N)cc2)cs1]

2-Ethynyl-N-(3-(methylsulfonyl)phenethyl)thiazole-4-carboxamide C105. ¹H NMR (400 MHz, DMSO-d₆) δ 8.68 (t, J=5.9 Hz, 1H), 8.34 (s, 1H), 7.80-7.72 (m, 2H), 7.62-7.52 (m, 2H), 5.04 (s, 1H), 3.54 (dd, J=13.2, 7.1 Hz, 5H), 2.97 (t, J=7.2 Hz, 2H); MS (ESI) m/z: 335.0 [M+H⁺].

N-(2-Chlorophenethyl)-2-ethynylthiazole-4-carboxamide C106. ¹H NMR (400 MHz, DMSO-d₆) δ 8.69 (t, J=5.9 Hz, 1H), 8.34 (s, 1H), 7.45-7.38 (m, 1H), 7.33 (dd, J=7.2, 2.2 Hz, 1H), 7.29-7.21 (m, 2H), 5.04 (s, 1H), 3.55-3.48 (m, 2H), 2.97 (t, J=7.3 Hz, 2H); MS (ESI) m/z: 291.1 [M+H⁺].

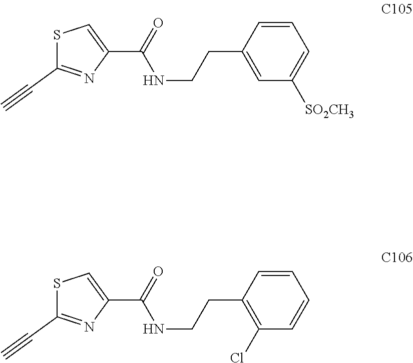 [SMILES: C#Cc1nc(C(=O)NCCc2cccc(S(C)(=O)=O)c2)cs1 ; C#Cc1nc(C(=O)NCCc2ccccc2Cl)cs1]

2-Ethynyl-N-(3-nitrobenzyl)thiazole-4-carboxamide C107. ¹H NMR (400 MHz, DMSO-d₆) δ 9.35 (t, J=6.0 Hz, 1H), 8.42 (s, 1H), 8.20 (s, 1H), 8.19-8.10 (m, 1H), 7.79 (d, J=4.0 Hz, 1H), 7.63 (t, J=8.0 Hz, 1H), 5.06 (s, 1H), 4.56 (d, J=4.0 Hz, 2H); MS (ESI) m/z: 288.3 [M+H⁺].

2-Ethynyl-N-(4-(methylsulfonyl)benzyl)thiazole-4-carboxamide C108. ¹H NMR (400 MHz, DMSO-d₆) δ 8.19 (s, 1H), 7.94-7.90 (m, 2H), 7.79 (s, 1H), 7.55 (d, J=8.0 Hz, 2H), 4.72 (d, J=8.0 Hz, 2H), 3.55 (s, 1H), 3.04 (s, 3H); MS (ESI) m/z: 321.0 [M+H⁺].

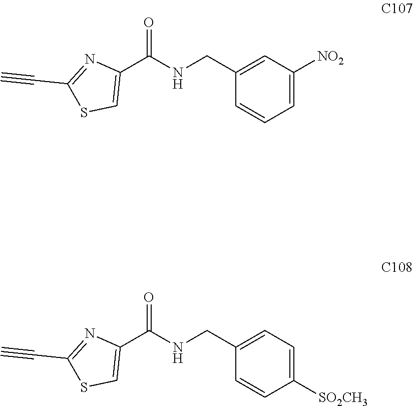 [SMILES: C#Cc1nc(C(=O)NCc2cccc([N+](=O)[O-])c2)cs1 ; C#Cc1nc(C(=O)NCc2ccc(S(C)(=O)=O)cc2)cs1]

N-((3-Chloropyridin-2-yl)methyl)-2-ethynylthiazole-4-carboxamide C109. ¹H NMR (400 MHz, DMSO-d₆) δ 8.89 (t, J=6.0 Hz, 1H), 8.53 (d, J=4.0 Hz, 1H), 8.43 (s, 1H), 7.99-7.96 (m, 1H), 7.42-7.39 (m, 1H), 5.08 (s, 1H), 4.69 (d, J=4.0 Hz, 2H); MS (ESI) m/z: 278.7 [M+H⁺].

N-(3-Chlorophenethyl)-2-ethynylthiazole-4-carboxamide C110. ¹H NMR (400 MHz, DMSO-d₆) δ 8.64 (t, J=5.8 Hz, 1H), 8.34 (s, 1H), 7.34-7.28 (m, 2H), 7.27-7.23 (m, 1H), 7.21-7.17 (m, 1H), 5.04 (s, 1H), 3.49 (dd, J=13.4, 7.1 Hz, 2H), 2.86 (t, J=7.2 Hz, 2H); MS (ESI) m/z: 291.0 [M+H⁺].

N-Benzyl-2-ethynyl-1H-imidazole-4-carboxamide C111. ¹H NMR (400 MHz, DMSO-d₆) δ 13.26 (s, 1H), 8.61 (s, 1H), 7.68 (s, 1H), 7.31-7.27 (m, 4H), 7.22 (d, J=5.5 Hz, 1H), 4.44 (s, 1H), 4.39 (d, J=6.2 Hz, 2H); MS (ESI) m/z: 226.1 [M+H⁺].

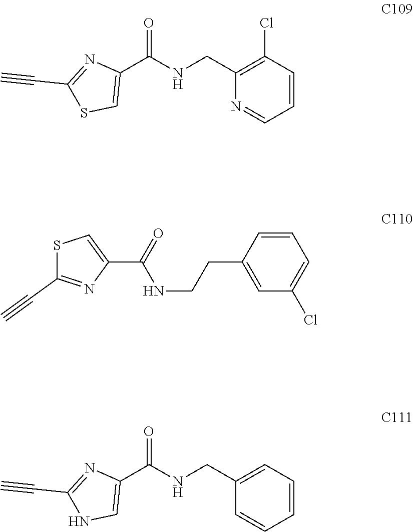 [SMILES: C#Cc1nc(C(=O)NCc2ncccc2Cl)cs1 ; C#Cc1nc(C(=O)NCCc2cccc(Cl)c2)cs1 ; C#Cc1nc(C(=O)NCc2ccccc2)c[nH]1]

N-([1,1′-Biphenyl]-2-ylmethyl)-2-ethynylthiazole-4-carboxamide C112. ¹H NMR (400 MHz, DMSO-d₆) δ 9.01 (t, J=6.0 Hz, 1H), 8.36 (s, 1H), 7.47-7.33 (m, 8H), 7.22-7.20 (m, 1H), 5.05 (s, 1H), 4.40 (d, J=6.1 Hz, 2H); MS (ESI) m/z: 319.0 [M+H⁺].

2-Ethynyl-N-(naphthalen-1-ylmethyl)thiazole-4-carboxamide C113. ¹H NMR (400 MHz, DMSO-d₆) δ 9.14 (t, J=6.0 Hz, 1H), 8.42 (s, 1H), 8.24 (d, J=8.2 Hz, 1H), 7.96-7.93 (m, 1H), 7.86-7.82 (m, 1H), 7.58-7.53 (m, 2H), 7.47 (d, J=5.1 Hz, 2H), 5.04 (s, 1H), 4.92 (d, J=6.1 Hz, 2H); MS (ESI) m/z: 293.1 [M+H⁺].

N-([1,1′-Biphenyl]-4-ylmethyl)-2-ethynylthiazole-4-carboxamide C114. ¹H NMR (400 MHz, DMSO-d₆) δ 9.18 (t, J=6.3 Hz, 1H), 8.40 (s, 1H), 7.65-7.60 (m, 4H), 7.46-7.35 (m, 5H), 5.05 (s, 1H), 4.48 (d, J=6.3 Hz, 2H); MS (ESI) m/z: 319.1 [M+H⁺].

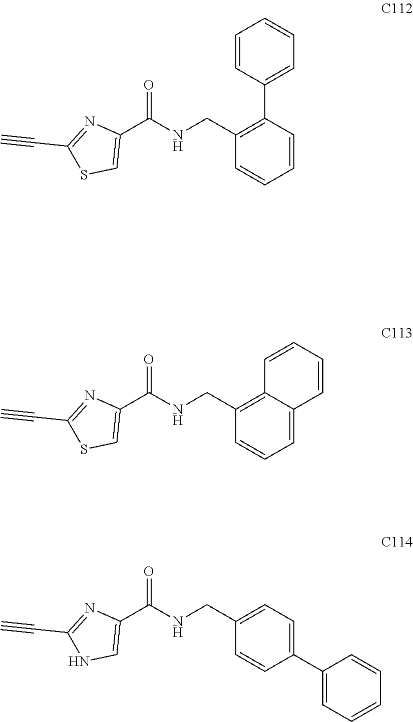 [SMILES: C#Cc1nc(C(=O)NCc2ccccc2-c2ccccc2)cs1 ; C#Cc1nc(C(=O)NCc2cccc3ccccc23)cs1 ; C#Cc1nc(C(=O)NCc2ccc(-c3ccccc3)cc2)c[nH]1]

N-([1,1′-Biphenyl]-3-ylmethyl)-2-ethynylthiazole-4-carboxamide C115. ¹H NMR (400 MHz, DMSO-d₆) δ 9.19 (t, J=6.3 Hz, 1H), 8.40 (s, 1H), 7.64-7.61 (m, 3H), 7.53-7.32 (m, 6H), 5.05 (s, 1H), 4.51 (d, J=6.3 Hz, 2H); MS (ESI) m/z: 319.0 [M+H⁺].

Ethyl 3-(N-benzyl-2-ethynylthiazole-4-carboxamido)propanoate C116. ¹H NMR (400 MHz, DMSO-d₆) δ 8.19 (s, 1H), 7.37-7.22 (m, 5H), 4.75 (s, 3H), 4.02 (q, J=7.1 Hz, 2H), 3.65 (s, 2H), 2.58 (t, J=7.2 Hz, 2H), 1.15 (t, J=7.1 Hz, 3H); MS (ESI) m/z: 343.1 [M+H⁺].

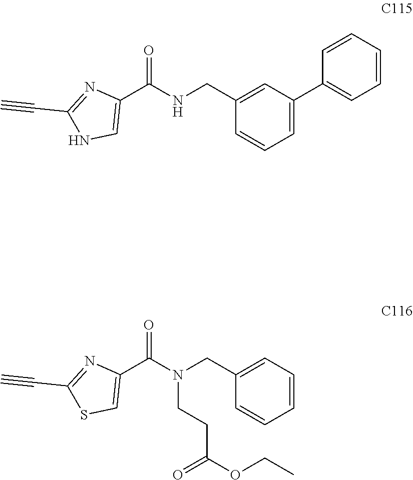 [SMILES: C#Cc1nc(C(=O)NCc2cccc(-c3ccccc3)c2)c[nH]1 ; C#Cc1nc(C(=O)N(CCC(=O)OCC)Cc2ccccc2)cs1]

Methyl 4-((2-ethynylthiazole-4-carboxamido)methyl)benzoate C117. ¹H NMR (400 MHz, DMSO-d₆) δ 9.25 (t, J=6.3 Hz, 1H), 8.41 (s, 1H), 7.93-7.90 (m, 2H), 7.44 (d, J=8.4 Hz, 2H), 5.06 (s, 1H), 4.51 (d, J=6.3 Hz, 2H), 3.84 (s, 3H); MS (ESI) m/z: 301.1 [M+H⁺].

N-(4-Aminobenzyl)-2-ethynylthiazole-4-carboxamide C118. ¹H NMR (400 MHz, DMSO-d₆) δ 8.84 (t, J=6.2 Hz, 1H), 8.36 (s, 1H), 6.98 (d, J=8.4 Hz, 2H), 6.51-6.48 (m, 2H), 5.03 (s, 1H), 4.25 (d, J=6.3 Hz, 2H); MS ESI m/z: 258.0 M+H⁺.

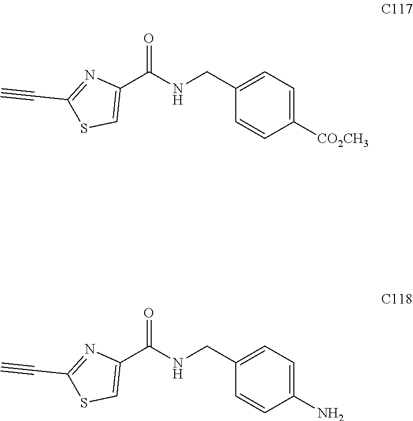 [SMILES: C#Cc1nc(C(=O)NCc2ccc(C(=O)OC)cc2)cs1 ; C#Cc1nc(C(=O)NCc2ccc(N)cc2)cs1]

2-Ethynyl-N-((1-methyl-1H-pyrazol-3-yl)methyl)thiazole-4-carboxamide C119. ¹H NMR (400 MHz, DMSO-d₆) δ 8.78 (t, J=5.7 Hz, 1H), 8.39 (s, 1H), 7.57 (d, J=1.9 Hz, 1H), 6.12 (d, J=2.0 Hz, 1H), 5.05 (s, 1H), 4.38 (d, J=6.0 Hz, 2H), 3.78 (s, 3H); MS (ESI) m/z: 247.1 [M+H⁺].

2-Ethynyl-N-((1-methyl-1H-pyrazol-4-yl)methyl)thiazole-4-carboxamide C120. ¹H NMR (400 MHz, DMSO-d₆) δ 8.86 (t, J=6.0 Hz, 1H), 8.36 (s, 1H), 7.57 (s, 1H), 7.33 (s, 1H), 5.03 (s, 1H), 4.25 (d, J=6.1 Hz, 2H), 3.77 (s, 3H); MS (ESI) m/z: 247.0 [M+H⁺].

2-Ethynyl-N-(4-hydroxybenzyl)thiazole-4-carboxamide C121. ¹H NMR (400 MHz, DMSO-d₆) δ 8.97 (t, J=6.3 Hz, 1H), 8.37 (s, 1H), 7.12 (d, J=8.4 Hz, 2H), 6.70-6.67 (m, 2H), 5.04 (s, 1H), 4.31 (d, J=6.3 Hz, 2H), 3.56 (s, 1H); MS (ESI) m/z: 259.1 [M+H⁺].

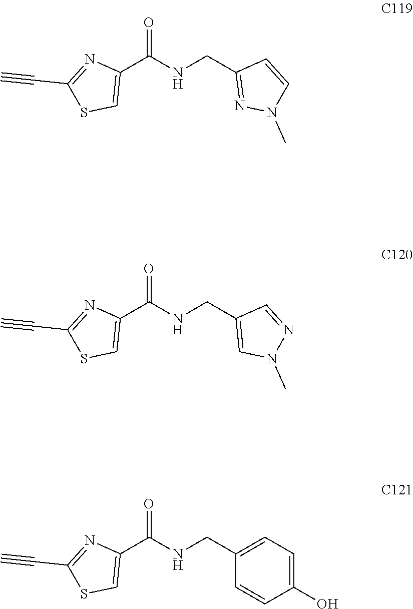 [SMILES: C#Cc1nc(C(=O)NCc2ccn(C)n2)cs1 ; C#Cc1nc(C(=O)NCc2cnn(C)c2)cs1 ; C#Cc1nc(C(=O)NCc2ccc(O)cc2)cs1]

2-Ethynyl-N-(4-hydroxy-3-methoxyphenethyl)thiazole-4-carboxamide C122. ¹H NMR (400 MHz, DMSO-d₆) δ 8.53 (t, J=5.8 Hz, 1H), 8.34 (s, 1H), 6.77 (d, J=1.8 Hz, 1H), 6.67 (d, J=7.9 Hz, 1H), 6.60 (dd, J=8.0, 1.8 Hz, 1H), 5.04 (s, 1H), 3.72 (s, 3H), 3.44 (dd, J=14.3, 6.5 Hz, 2H), 2.72 (t, J=7.4 Hz, 2H); MS (ESI) m/z: 303.1 [M+H⁺].

N-(2-Ethynylthiazol-4-yl)-3-phenylpropanamide C123. ¹H NMR (400 MHz, DMSO-d₆) δ 11.11 (s, 1H), 7.69 (s, 1H), 7.14-7.34 (m, 5H), 4.93 (s, 1H), 2.90 (d, J=5.6 Hz, 2H), 2.67 (d, J=5.6 Hz, 2H); MS (ESI) m/z: 257.1 [M+H⁺].

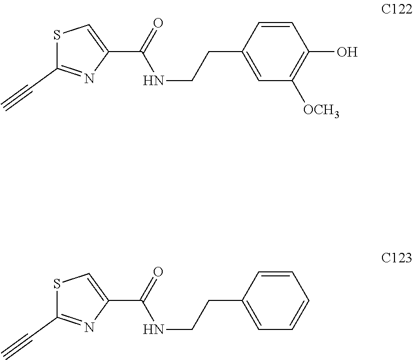 [SMILES: C#Cc1nc(C(=O)NCCc2ccc(O)c(OC)c2)cs1 ; C#Cc1nc(C(=O)NCCc2ccccc2)cs1]

Methyl 3-((2-ethynylthiazole-4-carboxamido)methyl)benzoate C124. ¹H NMR (400 MHz, DMSO-d₆) δ 8.18 (s, 1H), 8.01 (s, 1H), 7.96 (d, J=8.0 Hz, 1H), 7.70 (brs, 1H), 7.56 (d, J=8.0 Hz, 1H), 7.42 (t, J=8.0 Hz, 2H), 4.68 (d, J=4.0 Hz, 2H), 3.91 (s, 3H), 3.53 (s, 1H); MS (ESI) m/z: 301.3 [M+H⁺].

N-(3-Chlorobenzyl)-2-ethynylthiazole-4-carboxamide C125. ¹H NMR (400 MHz, DMSO-d₆) δ 9.23 (t, J=6.0 Hz, 1H), 8.41 (s, 1H), 7.37-7.28 (m, 4H), 5.05 (s, 1H), 4.45 (d, J=8.0 Hz, 2H); MS (ESI) m/z: 278.0 [M+H⁺].

2-Ethynyl-N-(pyridin-2-ylmethyl)thiazole-4-carboxamide C126. ¹H NMR (400 MHz, DMSO-d₆) δ 9.13 (t, J=6.0 Hz, 1H), 8.53 (d, J=8.0 Hz, 1H), 8.42 (s, 1H), 7.82-7.78 (m, 1H), 7.36-7.29 (m, 2H), 5.05 (t, J=4.0 Hz, 1H), 4.58 (d, J=8.0 Hz, 2H); MS (ESI) m/z: 244.0 [M+H⁺].

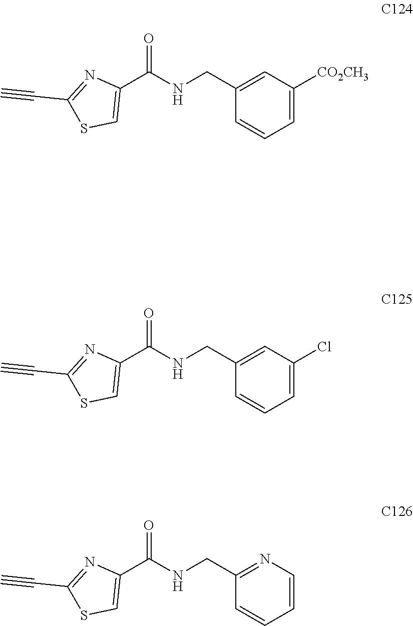 [SMILES: C#Cc1nc(C(=O)NCc2cccc(C(=O)OC)c2)cs1 ; C#Cc1nc(C(=O)NCc2cccc(Cl)c2)cs1 ; C#Cc1nc(C(=O)NCc2ccccn2)cs1]

N-(3-Cyanobenzyl)-2-ethynylthiazole-4-carboxamide C127. ¹H NMR (400 MHz, DMSO-d₆) δ 9.27 (t, J=6.0 Hz, 1H), 8.44 (s, 1H), 7.73-7.71 (m, 2H), 7.68 (d, J=8.0 Hz, 1H), 7.55 (t, J=8.0 Hz, 2H), 5.09 (s, 1H), 4.48 (d, J=8.0 Hz, 2H); MS (ESI) m/z: 268.1 [M+H⁺].

N-(4-Aminophenethyl)-2-ethynylthiazole-4-carboxamide C128. ¹H NMR (400 MHz, DMSO-d₆) δ 8.52 (t, J=5.9 Hz, 1H), 8.34 (s, 1H), 6.92 (d, J=8.3 Hz, 2H), 6.57 (d, J=8.2 Hz, 2H), 5.74 (s, 2H), 5.04 (s, 1H), 3.42-3.37 (m, 2H), 2.69-2.64 (m, 2H); MS (ESI) m/z: 272.1 [M+H⁺].

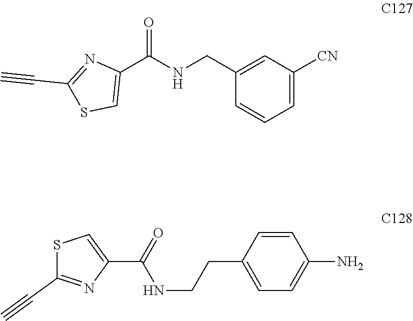 [SMILES: C#Cc1nc(C(=O)NCc2cccc(C#N)c2)cs1 ; C#Cc1nc(C(=O)NCCc2ccc(N)cc2)cs1]

2-Ethynyl-N-(4-nitrophenethyl)thiazole-4-carboxamide C129. ¹H NMR (400 MHz, DMSO-d₆) δ 8.69 (t, J=5.9 Hz, 1H), 8.34 (s, 1H), 8.15 (d, J=8.8 Hz, 2H), 7.51 (d, J=8.8 Hz, 2H), 5.04 (s, 1H), 3.55 (q, J=6.4 Hz, 2H), 3.00 (t, J=7.1 Hz, 2H); MS (ESI) m/z: 302.0 [M+H⁺].

2-Ethynyl-N-(1H-indazol-4-yl)thiazole-4-carboxamide C130. ¹H NMR (400 MHz, DMSO-d₆) δ 13.09 (s, 1H), 10.49 (s, 1H), 8.61 (s, 1H), 8.12 (s, 1H), 7.46 (dd, J=6.5, 1.7 Hz, 1H), 7.34 (t, J=3.9 Hz, 2H), 5.12 (s, 1H); MS (ESI) m/z: 269.0 [M+H⁺].

Ethyl N-benzyl-N-(2-ethynylthiazole-4-carbonyl)glycinate C131. ¹H NMR (400 MHz, DMSO-d₆) δ 10.68 (s, 1H), 9.79 (s, 1H), 8.73 (d, J=0.5 Hz, 1H), 8.50 (s, 1H), 7.93 (d, J=38.3 Hz, 2H), 7.49 (s, 1H), 7.04 (s, 1H), 3.84 (s, 3H), 3.51 (q, J=11.1 Hz, 2H); MS (ESI) m/z: 329.1 [M+H⁺].

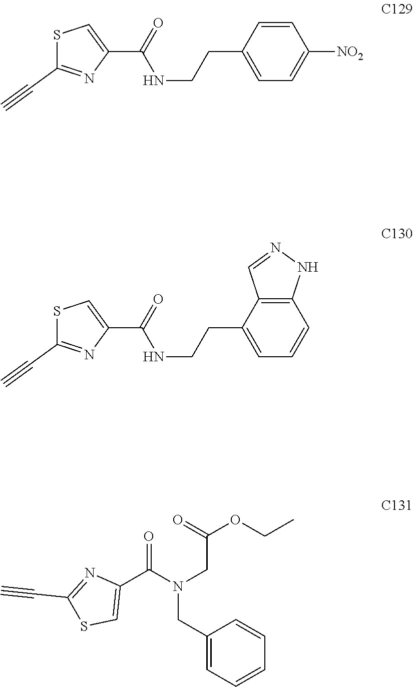 [SMILES: C#Cc1nc(C(=O)NCCc2ccc([N+](=O)[O-])cc2)cs1 ; C#Cc1nc(C(=O)NCCc2cccc3[nH]ncc23)cs1 ; C#Cc1nc(C(=O)N(CC(=O)OCC)Cc2ccccc2)cs1]

N-Benzyl-2-ethynyl-1-methyl-1H-imidazole-5-carboxamide C132. ¹H NMR (400 MHz, DMSO-d₆) δ 8.44 (t, J=5.8 Hz, 1H), 7.77 (s, 1H), 7.42-7.28 (m, 5H), 7.27-7.22 (m, 1H), 4.48 (d, J=6.0 Hz, 2H), 4.42 (s, 1H), 3.75 (s, 3H); MS (ESI) m/z: 240.1 [M+H⁺].

N-Benzyl-2-ethynyl-N-(2-hydroxyethyl)thiazole-4-carboxamide C133. ¹H NMR (400 MHz, DMSO-d₆) δ 8.16 (s, 1H), 7.37-7.26 (m, 6H), 4.82 (s, 2H), 4.77 (s, 1H), 3.57 (t, J=6.0 Hz, 2H), 3.50 (t, J=6.0 Hz, 2H); MS (ESI) m/z: 287.1 [M+H⁺].

2-Ethynyl-N-(11H-indazol-7-yl)thiazole-4-carboxamide C134. ¹H NMR (400 MHz, DMSO-d₆) δ 12.92 (s, 1H), 10.49 (s, 1H), 8.59 (s, 1H), 8.10 (d, J=1.3 Hz, 1H), 7.63 (d, J=8.0 Hz, 1H), 7.56 (d, J=7.3 Hz, 1H), 7.11 (t, J=7.7 Hz, 1H), 5.11 (s, 1H); MS (ESI) m/z: 269.0 M+H⁺.

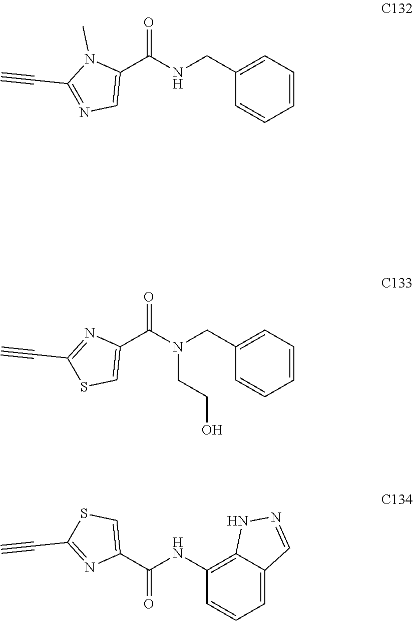 [SMILES: C#Cc1ncc(C(=O)NCc2ccccc2)n1C ; C#Cc1nc(C(=O)N(CCO)Cc2ccccc2)cs1 ; C#Cc1nc(C(=O)Nc2cccc3cn[nH]c23)cs1]

(S)-2-Ethynyl-N-(2-hydroxy-2-phenylethyl)thiazole-4-carboxamide C135. ¹H NMR (400 MHz, DMSO-d₆) δ 8.15 (s, 1H), 7.74 (brs, 1H), 7.43-7.35 (m, 4H), 7.32-7.28 (m, 1H), 4.95 (d, J=8.0 Hz, 1H), 3.88-3.82 (m, 1H), 3.59-3.52 (m, 3H), 3.31 (s, 1H); MS (ESI) m/z: 273.1 [M+H⁺].

N-(2-Acetamidobenzyl)-2-ethynylthiazole-4-carboxamide C136. ¹H NMR (400 MHz, DMSO-d₆) δ 9.79 (s, 1H), 9.21 (t, J=6.3 Hz, 1H), 8.44 (s, 1H), 7.55 (d, J=7.7 Hz, 1H), 7.33 (dd, J=7.6, 1.2 Hz, 1H), 7.23 (t, J=7.0 Hz, 1H), 7.10 (d, J=7.4 Hz, 1H), 5.06 (s, 1H), 4.41 (d, J=6.4 Hz, 2H), 2.11 (s, 3H); MS (ESI) m/z: 300.1 [M+H⁺].

N-Benzyl-2-ethynyl-N-methylthiazole-4-carboxamide C137. ¹H NMR (400 MHz, DMSO-d₆) δ 8.20 (s, 1H), 7.37-7.27 (m, 5H), 4.82 (s, 1H), 4.72 (s, 2H), 3.10 (s, 3H); MS (ESI) m/z: 258.1 [M+H⁺].

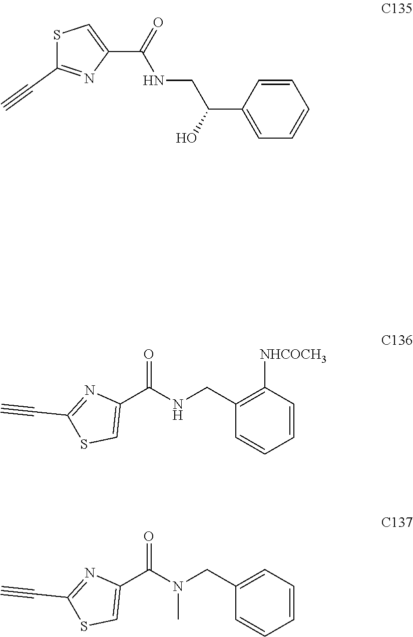

2-Ethynyl-N-phenethylthiazole-4-carboxamide C138. ¹H NMR (400 MHz, DMSO-d₆) δ 8.12 (s, 1H), 7.39 (brs, 1H), 7.33-7.30 (m, 2H), 7.25-7.21 (m, 3H), 3.52 (s, 1H), 2.92 (t, J=6.0 Hz, 2H); MS (ESI) m/z: 257.1 [M+H⁺].

N-((1H-Indol-4-yl)methyl)-2-ethynylthiazole-4-carboxamide C139. ¹H NMR (400 MHz, DMSO-d₆) δ 11.10 (s, 1H), 8.95 (t, J=8.0 Hz, 1H), 8.39 (s, 1H), 7.32-7.28 (m, 2H), 7.02 (t, J=8.0 Hz, 1H), 6.91 (d, J=8.0 Hz, 1H), 6.61 (t, J=4.0 Hz, 1H), 5.02 (s, 1H), 4.70 (d, J=8.0 Hz, 2H); MS (ESI) m/z: 282.2 [M+H⁺].

N-(2-Aminobenzyl)-2-ethynylthiazole-4-carboxamide C140. ¹H NMR (400 MHz, DMSO-d₆) δ 9.02 (t, J=6.3 Hz, 1H), 8.39 (s, 1H), 7.06 (dd, J=7.5, 1.3 Hz, 1H), 6.94 (td, J=7.9, 1.5 Hz, 1H), 6.60 (dd, J=7.9, 0.9 Hz, 1H), 6.48 (td, J=7.4, 1.1 Hz, 1H), 5.18 (s, 2H), 5.04 (s, 1H), 4.27 (d, J=6.4 Hz, 2H); MS (ESI) m/z: 258.1 [M+H⁺].

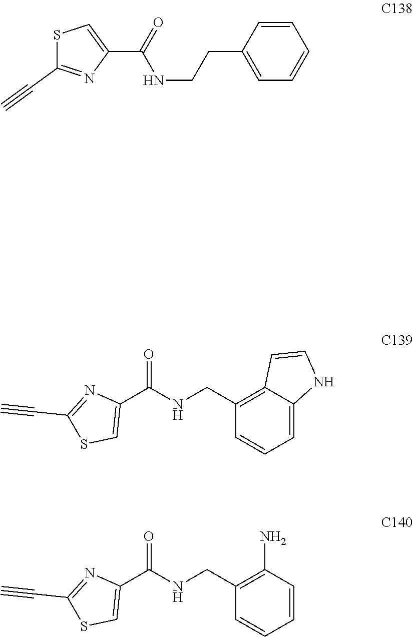 [SMILES: C#Cc1nc(C(=O)NCCc2ccccc2)cs1 ; C#Cc1nc(C(=O)NCc2cccc3[nH]ccc23)cs1 ; C#Cc1nc(C(=O)NCc2ccccc2N)cs1]

N-Benzyl-2-ethynylthiazole-5-carboxamide C141. ¹H NMR (400 MHz, DMSO-d₆) δ 8.11 (s, 1H), 7.32-7.42 (m, 5H), 6.30 (brs, 1H), 4.64 (d, J=5.6 Hz, 1H), 3.59 (s, 1H); MS (ESI) m/z: 243.0 [M+H⁺].

N-Benzyl-2-ethynyloxazole-4-carboxamide C142. ¹H NMR (400 MHz, DMSO-d₆) δ 9.02 (t, J=6.2 Hz, 1H), 8.71 (s, 1H), 7.34-7.20 (m, 6H), 4.98 (s, 1H), 4.41 (d, J=6.3 Hz, 2H); MS (ESI) m/z: 227.0 [M+H⁺].

N-(2-Chlorobenzyl)-2-ethynylthiazole-4-carboxamide C143. ¹H NMR (400 MHz, DMSO-d₆) δ 9.13 (t, J=6.1 Hz, 1H), 8.43 (s, 1H), 7.46-7.43 (m, 1H), 7.32-7.28 (m, 3H), 5.07 (s, 1H), 4.51 (d, J=6.2 Hz, 2H); MS (ESI) m/z: 277.0 [M+H⁺].

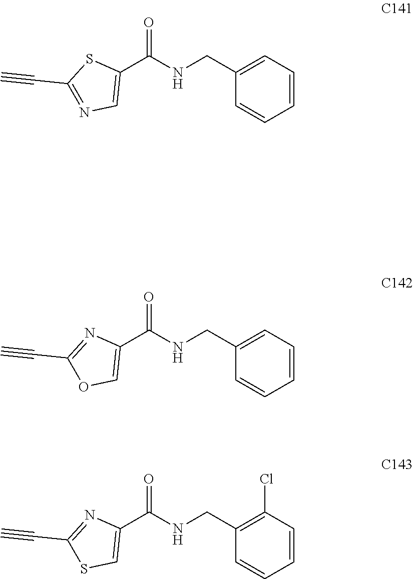 [SMILES: C#Cc1ncc(C(=O)NCc2ccccc2)s1 ; C#Cc1nc(C(=O)NCc2ccccc2)co1 ; C#Cc1nc(C(=O)NCc2ccccc2Cl)cs1]

2-Ethynyl-N-(2-methoxybenzyl)thiazole-4-carboxamide C144. ¹H NMR (400 MHz, DMSO-d₆) δ 8.84 (t, J=6.1 Hz, 1H), 8.40 (s, 1H), 7.23 (td, J=8.2, 1.6 Hz, 1H), 7.17-7.11 (m, 1H), 6.99 (d, J=7.7 Hz, 1H), 6.89 (td, J=7.4, 0.8 Hz, 1H), 5.06 (s, 1H), 4.43 (d, J=6.2 Hz, 2H), 3.83 (s, 3H); MS (ESI) m/z: 273.1 [M+H⁺].

N-Benzyl-4-ethynylthiazole-2-carboxamide C145. ¹H NMR (400 MHz, DMSO-d₆) δ 9.56 (t, J=6.1 Hz, 1H), 8.32 (s, 1H), 7.32 (d, J=4.4 Hz, 4H), 7.25 (dd, J=8.8, 4.5 Hz, 1H), 4.44 (d, J=6.4 Hz, 2H), 4.39 (s, 1H); MS (ESI) m/z: 243.0 [M+H⁺].

N-Benzyl-2-ethynylpyrimidine-4-carboxamide C146.

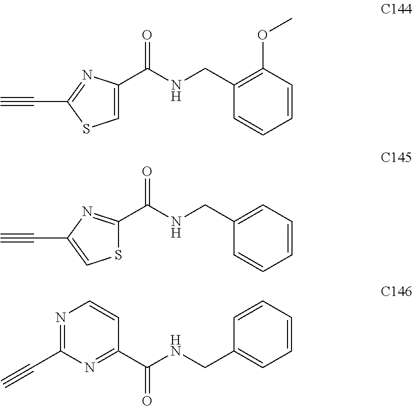

N-Benzyl-6-ethynylpicolinamide C147.

N-Benzyl-2-ethynylthiazole-4-carboxamide C148. ¹H NMR (400 MHz, DMSO-d₆) δ 8.17 (s, 1H), 7.35 (d, J=4.4 Hz, 4H), 7.31-7.27 (m, 1H), 4.63 (d, J=6.0 Hz, 2H), 3.51 (s, 1H); MS (ESI) m/z: 243.1 [M+H⁺].

N-Benzyl-2-ethynylisonicotinamide C149.

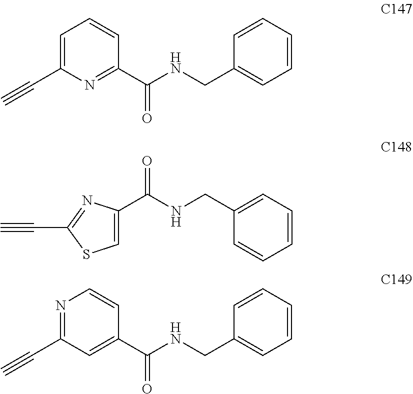

(4-(4-(1,5-Dimethyl-1H-indazol-4-yl)phenyl)piperazin-1-yl)(2-ethynylthiazol-4-yl)methanone D1. ¹H NMR (400 MHz, DMSO-d₆) δ 8.30 (s, 1H), 7.58 (d, J=0.9 Hz, 1H), 7.50-7.47 (m, 1H), 7.33-7.29 (m, 3H), 7.12-7.09 (m, 2H), 5.04 (s, 1H), 4.02 (s, 3H), 3.83 (s, 4H), 3.35-3.26 (m, 4H), 2.30 (s, 3H); MS (ESI) m/z: 442.1 [M+H⁺].

Methyl 4-(4-(4-(2-ethynylthiazole-4-carbonyl)piperazin-1-yl)phenyl)-1-methyl-1H-indazole-6-carboxylate D2. ¹H NMR (400 MHz, DMSO-d₆) δ 8.30 (s, 1H), 8.25-8.20 (m, 2H), 7.72 (d, J=1.2 Hz, 1H), 7.66 (d, J=8.8 Hz, 2H), 7.15 (d, J=8.8 Hz, 2H), 5.04 (s, 1H), 4.17 (s, 3H), 3.93 (s, 3H), 3.32 (s, 9H); MS (ESI) m/z: 486.1 [M+H⁺].

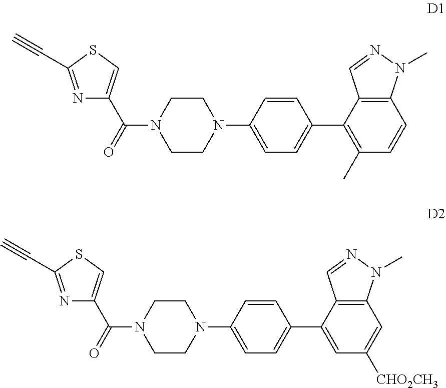

7-(4-(4-(2-Ethynylthiazole-4-carbonyl)piperazin-1-yl)phenyl)-N-methylbenzo[d]thiazole-5-carboxamide D3. ¹H NMR (400 MHz, DMSO-d₆) δ 9.51 (s, 1H), 8.71 (d, J=4.6 Hz, 1H), 8.48 (d, J=1.5 Hz, 1H), 8.30 (s, 1H), 8.00 (d, J=1.4 Hz, 1H), 7.67 (d, J=8.8 Hz, 2H), 7.16 (d, J=8.9 Hz, 2H), 5.04 (s, 1H), 3.35 (s, 9H), 2.85 (d, J=4.5 Hz, 3H); MS (ESI) m/z: 488.1 [M+H⁺].

Methyl 7-(4-(4-(2-ethynylthiazole-4-carbonyl)piperazin-1-yl)phenyl)benzo[d]-thiazole-5-carboxylate D4. ¹H NMR (400 MHz, DMSO-d₆) δ 9.57 (s, 1H), 8.54 (d, J=1.5 Hz, 1H), 8.30 (s, 1H), 8.02 (d, J=1.4 Hz, 1H), 7.65 (d, J=8.8 Hz, 2H), 7.16 (d, J=8.9 Hz, 2H), 5.04 (s, 1H), 3.94 (s, 3H), 3.32 (s, 8H); MS (ESI) m/z: 489.1 [M+H⁺].

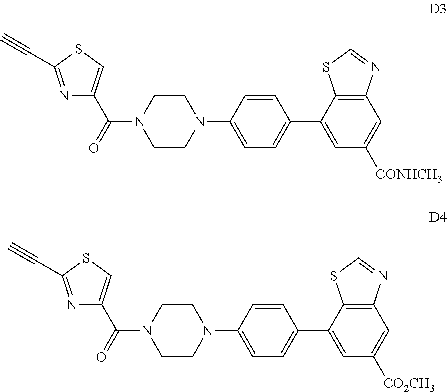

7-(4-(4-(2-Ethynylthiazole-4-carbonyl)piperazin-1-yl)phenyl)benzo[d]thiazole-5-carboxamide D5. ¹H NMR (400 MHz, DMSO-d₆) δ 9.51 (s, 1H), 8.54 (d, J=1.5 Hz, 1H), 8.30 (s, 1H), 8.25 (s, 1H), 8.04 (d, J=1.4 Hz, 1H), 7.67 (d, J=8.8 Hz, 2H), 7.54 (s, 1H), 7.17 (d, J=8.9 Hz, 2H), 5.04 (s, 1H), 3.84 (s, 4H), 3.35 (d, J=18.7 Hz, 4H); MS (ESI) m/z: 474.1 [M+H⁺].

7-(4-(4-(2-Ethynylthiazole-4-carbonyl)piperazin-1-yl)phenyl)-N,N-dimethyl-benzo[d]thiazole-5-carboxamide D6. ¹H NMR (400 MHz, DMSO-d₆) δ 9.51 (s, 1H), 8.30 (s, 1H), 8.04 (d, J=1.4 Hz, 1H), 7.64 (d, J=8.8 Hz, 2H), 7.53 (d, J=1.3 Hz, 1H), 7.15 (d, J=8.9 Hz, 2H), 5.04 (s, 1H), 3.83 (s, 4H), 3.32 (s, 4H), 3.02 (d, J=15.0 Hz, 6H); MS (ESI) m/z: 502.1 [M+H⁺].

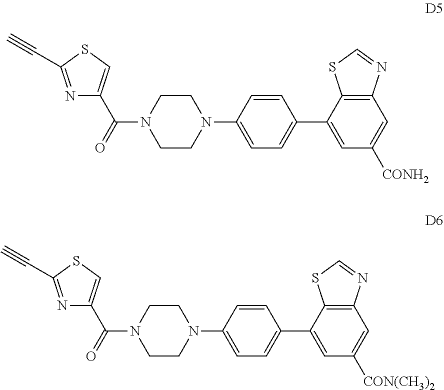

(4-(4-(Benzo[d]thiazol-4-yl)phenyl)piperazin-1-yl)(2-ethynylthiazol-5-yl)methanone D8. ¹H NMR (400 MHz, DMSO-d₆) δ 9.47 (s, 1H), 8.30 (s, 1H), 8.12 (dd, J=7.9, 1.2 Hz, 1H), 8.07 (d, J=2.3 Hz, 1H), 7.98 (dd, J=8.6, 2.3 Hz, 1H), 7.74-7.58 (m, 2H), 7.37 (d, J=8.7 Hz, 1H), 5.04 (s, 1H), 3.87 (s, 4H), 3.30 (s, 4H); MS (ESI) m/z: 431.1 [M+H⁺].

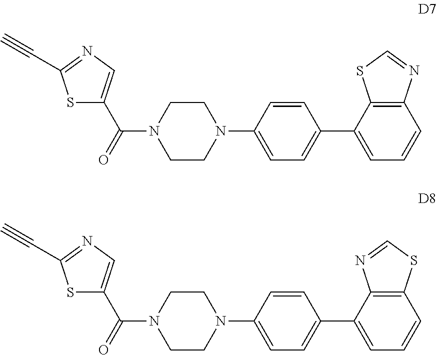 [SMILES: C#Cc1ncc(C(=O)N2CCN(c3ccc(-c4cccc5ncsc45)cc3)CC2)s1 ; C#Cc1ncc(C(=O)N2CCN(c3ccc(-c4cccc5scnc45)cc3)CC2)s1]

(4-(4-(Benzo[d]thiazol-7-yl)-2-chlorophenyl)piperazin-1-yl)(2-ethynylthiazol-4-yl)methanone D9. ¹H NMR (400 MHz, DMSO-d₆) δ 9.45 (s, 1H), 8.29 (s, 1H), 8.11 (dd, J=8.0, 0.9 Hz, 1H), 7.78 (d, J=1.9 Hz, 1H), 7.71-7.63 (m, 2H), 7.60 (d, J=7.3 Hz, 1H), 7.36 (d, J=8.3 Hz, 1H), 5.04 (s, 1H), 3.87-3.83 (m, 4H), 3.13 (d, J=11.4 Hz, 4H); MS (ESI) m/z: 465.0 [M+H⁺].

(4-(5-(Benzo[d]thiazol-7-yl)pyridin-2-yl)piperazin-1-yl)(2-ethynylthiazol-4-yl)-methanone D10. ¹H NMR (400 MHz, DMSO-d₆) δ 9.44 (s, 1H), 8.51 (d, J=2.2 Hz, 1H), 8.28 (s, 1H), 8.07 (d, J=8.0 Hz, 1H), 7.94 (dd, J=8.8, 2.3 Hz, 1H), 7.65 (t, J=7.8 Hz, 1H), 7.57 (d, J=7.3 Hz, 1H), 7.05 (d, J=8.9 Hz, 1H), 5.01 (s, 1H), 3.84-3.76 (m, 8H); MS (ESI) m/z: 432.1 [M+H⁺]

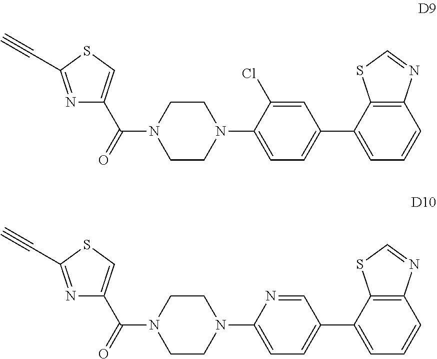 [SMILES: C#Cc1nc(C(=O)N2CCN(c3ccc(-c4cccc5ncsc45)cc3Cl)CC2)cs1 ; C#Cc1nc(C(=O)N2CCN(c3ccc(-c4cccc5ncsc45)cn3)CC2)cs1]

(4-(4-(Benzo[d]thiazol-7-yl)phenyl)-4-fluoropiperidin-1-yl)(2-ethynylthiazol-4-yl)methanone D11. ¹H NMR (400 MHz, DMSO-d₆) δ 9.46 (s, 1H), 8.29 (s, 1H), 8.12 (dd, J=8.0, 1.0 Hz, 1H), 7.77 (d, J=8.2 Hz, 2H), 7.70-7.64 (m, 3H), 7.61 (d, J=7.4 Hz, 1H), 5.03 (s, 1H), 4.58 (d, J=11.7 Hz, 1H), 4.18 (d, J=11.3 Hz, 1H), 3.55-3.40 (m, 1H), 3.16 (t, J=11.4 Hz, 1H), 2.13 (dd, J=58.5, 31.0 Hz, 4H); MS (ESI) m/z: 448.1 [M+H⁺].

5-(Benzo[d]thiazol-7-yl)-2-(4-(2-ethynylthiazole-4-carbonyl)piperazin-1-yl)-benzonitrile D12. ¹H NMR (400 MHz, DMSO-d₆) δ 9.47 (s, 1H), 8.30 (s, 1H), 8.12 (dd, J=7.9, 1.2 Hz, 1H), 8.07 (d, J=2.3 Hz, 1H), 7.98 (dd, J=8.6, 2.3 Hz, 1H), 7.74-7.58 (m, 2H), 7.37 (d, J=8.7 Hz, 1H), 5.04 (s, 1H), 3.87 (s, 4H), 3.30 (s, 4H); MS (ESI) m/z: 456.1 [M+H⁺].

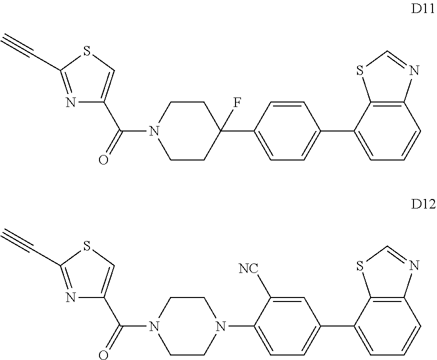 [SMILES: C#Cc1nc(C(=O)N2CCC(F)(c3ccc(-c4cccc5ncsc45)cc3)CC2)cs1 ; C#Cc1nc(C(=O)N2CCN(c3ccc(-c4cccc5ncsc45)cc3C#N)CC2)cs1]

(3-(4-(Benzo[d]thiazol-7-yl)phenyl)azetidin-1-yl)(2-ethynylthiazol-4-yl)-methanone D13. ¹H NMR (400 MHz, DMSO-d₆) δ 9.46 (s, 1H), 8.46 (s, 1H), 8.11 (dd, J=8.0, 1.1 Hz, 1H), 7.76-7.71 (m, 2H), 7.71-7.64 (m, 1H), 7.60 (d, J=8.4 Hz, 3H), 5.02 (d, J=2.2 Hz, 1H), 4.62-4.46 (m, 2H), 4.18-3.99 (m, 2H); MS (ESI) m/z: 402.0 [M+H⁺].

(4-(4-(Benzo[d]thiazol-7-yl)phenyl)-4-hydroxypiperidin-1-yl)(2-ethynylthiazol-4-yl)methanone D14. ¹H NMR (400 MHz, DMSO-d₆) δ 9.46 (s, 1H), 8.24 (s, 1H), 8.09-8.13 (m, 1H), 7.58-7.72 (m, 6H), 5.36 (s, 1H), 5.02 (s, 1H), 4.42-4.52 (m, 1H), 3.91-4.00 (m, 1H), 3.48-3.61 (m, 1H), 3.17-3.29 (m, 1H), 1.94-2.08 (m, 2H), 1.64-1.96 (m, 2H); MS (ESI) m/z: 446.0 [M+H⁺].

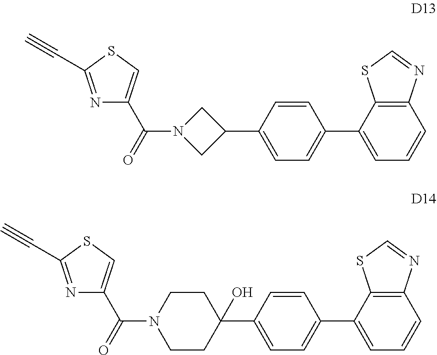 [SMILES: C#Cc1nc(C(=O)N2CC(c3ccc(-c4cccc5ncsc45)cc3)C2)cs1 ; C#Cc1nc(C(=O)N2CCC(O)(c3ccc(-c4cccc5ncsc45)cc3)CC2)cs1]

(4-(3-(Benzo[d]thiazol-7-yl)phenyl)piperidin-1-yl)(2-ethynylthiazol-4-yl)-methanone D15. ¹H NMR (400 MHz, DMSO-d₆) δ 9.43 (s, 1H), 8.19 (s, 1H), 8.11 (dd, J=8.0, 1.1 Hz, 1H), 7.68 (t, 1H), 7.64-7.60 (m, 2H), 7.59-7.56 (m, 1H), 7.51 (t, J=7.6 Hz, 1H), 7.41-7.38 (m, 1H), 4.95 (s, 1H), 4.64 (d, J=12.4 Hz, 1H), 4.13 (d, J=11.8 Hz, 1H), 3.25 (t, J=11.9 Hz, 1H), 3.01-2.89 (m, 2H), 1.99 (d, J=12.8 Hz, 1H), 1.88 (d, J=11.3 Hz, 1H), 1.74-1.64 (m, 2H); MS (ESI) m/z: 430.2 [M+H⁺].

(4-(3-(Benzo[d]thiazol-7-yl)phenyl)piperazin-1-yl)(2-ethynylthiazol-4-yl)-methanone D16. ¹H NMR (400 MHz, DMSO-d₆) δ 9.45-9.40 (m, 1H), 8.28 (s, 1H), 8.09 (dd, J=7.9, 1.2 Hz, 1H), 7.66 (t, J=7.7 Hz, 1H), 7.62-7.59 (m, 1H), 7.41 (t, J=7.9 Hz, 1H), 7.27-7.25 (m, 1H), 7.15 (d, J=8.0 Hz, 1H), 7.09 (dd, J=8.2, 2.1 Hz, 1H), 5.03 (s, 1H), 3.82 (s, 4H), 3.31-3.25 (m, 4H); MS (ESI) nm/z: 431.1 [M+H⁺].

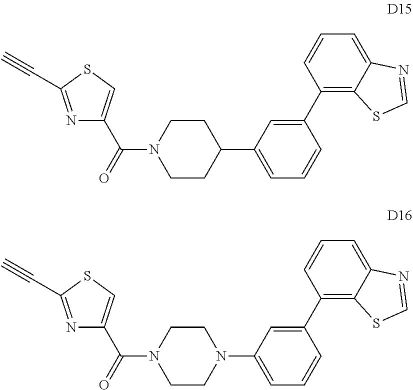 [SMILES: C#Cc1nc(C(=O)N2CCC(c3cccc(-c4cccc5ncsc45)c3)CC2)cs1 ; C#Cc1nc(C(=O)N2CCN(c3cccc(-c4cccc5ncsc45)c3)CC2)cs1]

(4-(4-(Benzo[d]thiazol-7-yl)phenyl)piperazin-1-yl)(2-ethynylthiazol-4-yl)-methanone D17. ¹H NMR (400 MHz, DMSO-d₆) δ 9.43 (s, 1H), 8.30 (s, 1H), 8.04 (dd, J=8.0, 1.0 Hz, 1H), 7.62 (dd, J=12.2, 4.8 Hz, 3H), 7.54 (d, J=6.8 Hz, 1H), 7.14 (d, J=8.8 Hz, 2H), 5.04 (s, 1H), 3.83 (s, 4H), 3.33 (s, 4H); MS (ESI) m/z: 431.1 [M+H⁺].

(4-(4-(Benzo[d]thiazol-7-yl)phenyl)piperidin-1-yl)(2-ethynylthiazol-4-yl)-methanone D18. ¹H NMR (400 MHz, DMSO-d₆) δ 9.45 (s, 1H), 8.23 (s, 1H), 8.10 (dd, J=8.0, 1.0 Hz, 1H), 7.67 (dd, J=12.0, 5.1 Hz, 3H), 7.58 (d, J=7.3 Hz, 1H), 7.47 (d, J=8.2 Hz, 2H), 5.02 (s, 1H), 4.65 (d, J=12.2 Hz, 1H), 4.16 (d, J=11.8 Hz, 1H), 3.25 (dd, J=23.3, 10.6 Hz, 1H), 2.95 (t, J=12.2 Hz, 2H), 1.92 (dd, J=38.0, 11.2 Hz, 2H), 1.78-1.54 (m, 2H); MS (ESI) m/z: 430.0 [M+H⁺].

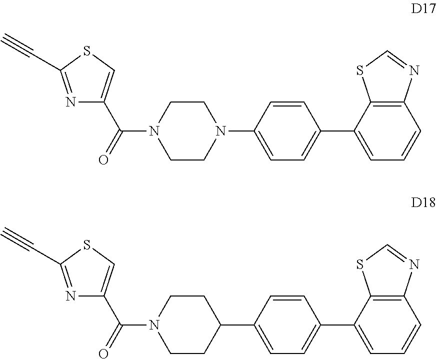 [SMILES: C#Cc1nc(C(=O)N2CCN(c3ccc(-c4cccc5ncsc45)cc3)CC2)cs1 ; C#Cc1nc(C(=O)N2CCC(c3ccc(-c4cccc5ncsc45)cc3)CC2)cs1]

(2-Ethynylthiazol-4-yl)(4-(4-(2-methyl-2H-indazol-4-yl)phenyl)piperidin-1-yl)-methanone D19. ¹H NMR (400 MHz, DMSO-d₆) δ 8.50 (s, 1H), 8.23 (s, 1H), 7.66 (d, J=8.2 Hz, 2H), 7.57 (d, J=8.6 Hz, 1H), 7.41 (d, J=8.2 Hz, 2H), 7.30 (dd, J=8.6, 6.9 Hz, 1H), 7.14 (d, J=6.5 Hz, 1H), 5.02 (s, 1H), 4.65 (d, J=10.9 Hz, 1H), 4.18 (s, 3H), 4.14 (s, 1H), 3.28-3.18 (m, 1H), 2.98-2.85 (m, 2H), 2.02-1.80 (m, 2H), 1.67 (dd, J=21.5, 12.1 Hz, 2H); MS (ESI) m/z: 427.1 [M+H⁺].

(2-Ethynylthiazol-4-yl)(4-(4-(1-methyl-1H-indazol-4-yl)phenyl)piperidin-1-yl)-methanone D20. ¹H NMR (400 MHz, DMSO-d₆) δ 8.24 (s, 1H), 8.13 (d, J=0.8 Hz, 1H), 7.65 (dd, J=19.5, 8.3 Hz, 3H), 7.50-7.41 (m, 3H), 7.24 (d, J=6.8 Hz, 1H), 5.02 (s, 1H), 4.65 (d, J=12.4 Hz, 1H), 4.15 (d, J=13.0 Hz, 1H), 4.09 (s, 3H), 3.24 (t, J=12.0 Hz, 1H), 2.99-2.84 (m, 2H), 1.91 (dd, J=38.1, 10.5 Hz, 2H), 1.67 (d, J=11.9 Hz, 2H); MS (ESI) m/z: 427.1 [M+H⁺].

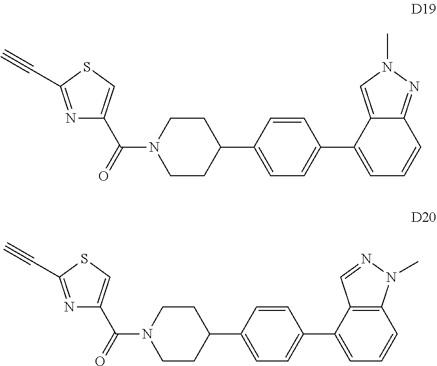 [SMILES: C#Cc1nc(C(=O)N2CCC(c3ccc(-c4cccc5nn(C)cc45)cc3)CC2)cs1 ; C#Cc1nc(C(=O)N2CCC(c3ccc(-c4cccc5c4cnn5C)cc3)CC2)cs1]

(4-(4-(1H-Indazol-4-yl)phenyl)piperidin-1-yl)(2-ethynylthiazol-4-yl)methanone D21. ¹H NMR (400 MHz, DMSO-d₆) δ 8.23 (d, J=4.4 Hz, 1H), 8.16 (d, J=0.9 Hz, 1H), 7.68 (d, J=8.2 Hz, 2H), 7.53 (d, J=8.3 Hz, 1H), 7.43 (dd, J=12.3, 5.3 Hz, 3H), 7.21 (d, J=7.0 Hz, 1H), 5.02 (s, 1H), 4.65 (d, J=12.1 Hz, 1H), 4.15 (d, J=11.5 Hz, 1H), 3.24 (t, J=12.9 Hz, 1H), 2.93 (dd, J=16.1, 7.4 Hz, 2H), 2.03-1.81 (m, 2H), 1.74-1.59 (m, 2H); MS (ESI) m/z: 413.1 [M+H⁺].

(2-Ethynylthiazol-4-yl)(4-(4-(1-methyl-1H-indazol-4-yl)phenyl)piperazin-1-yl)-methanone D22. ¹H NMR (400 MHz, DMSO-d₆) δ 8.30 (s, 1H), 8.13 (s, 1H), 7.63 (d, J=8.8 Hz, 2H), 7.57 (d, J=8.4 Hz, 1H), 7.42-7.47 (m, 1H), 7.20 (d, J=7.2 Hz, 1H), 7.13 (d, J=8.4 Hz, 2H), 5.05 (s, 1H), 4.08 (s, 3H), 3.77-3.90 (m, 4H), 3.22-3.32 (m, 4H); MS (ESI) m/z: 428.0 [M+H⁺].

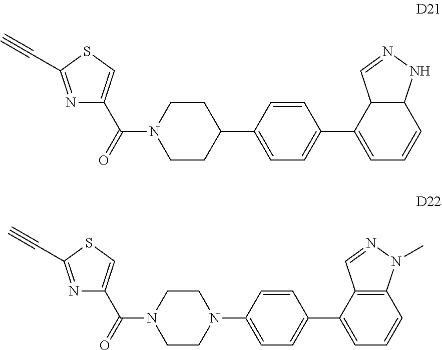 [SMILES: C#Cc1nc(C(=O)N2CCC(c3ccc(C4=CC=CC5NN=CC45)cc3)CC2)cs1 ; C#Cc1nc(C(=O)N2CCN(c3ccc(-c4cccc5c4cnn5C)cc3)CC2)cs1]

(2-Ethynylthiazol-4-yl)(4-(4-(2-methyl-2H-indazol-4-yl)phenyl)piperazin-1-yl)-methanone D23. ¹H NMR (400 MHz, DMSO-d₆) δ 8.48 (s, 1H), 8.30 (s, 1H), 7.62 (d, J=8.8 Hz, 2H), 7.51 (d, J=8.8 Hz, 1H), 7.26-7.31 (m, 1H), 7.17-7.14 (m, 3H), 5.05 (s, 1H), 4.18 (s, 3H), 3.79-3.90 (m, 4H), 3.22-3.32 (m, 4H); MS (ESI) m/z: 428.0 [M+H⁺].

4-(1-(2-Ethynylthiazole-4-carbonyl)piperidin-4-yl)benzonitrile D24. ¹H NMR (400 MHz, DMSO-d₆) δ 8.29 (s, 1H), 7.61 (d, J=9.0 Hz, 2H), 7.03 (d, J=9.1 Hz, 2H), 5.03 (s, 1H), 3.79 (d, J=18.3 Hz, 4H), 3.44 (d, J=20.5 Hz, 4H); MS (ESI) m/z: 322.1 [M+H⁺].

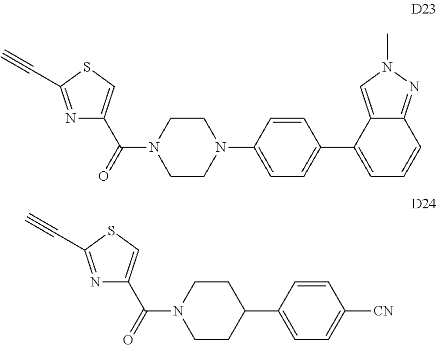 [SMILES: C#Cc1nc(C(=O)N2CCN(c3ccc(-c4cccc5nn(C)cc45)cc3)CC2)cs1 ; C#Cc1nc(C(=O)N2CCC(c3ccc(C#N)cc3)CC2)cs1]

(4-(4-(1H-Indazol-4-yl)phenyl)piperazin-1-yl)(2-ethynylthiazol-4-yl)methanone D25. ¹H NMR (400 MHz, DMSO-d₆) δ 13.17 (s, 1H), 8.30 (s, 1H), 8.16 (s, 1H), 7.64 (d, J=8.8 Hz, 2H), 7.47 (d, J=8.4 Hz, 1H), 7.37-7.43 (m, 1H), 7.10-7.20 (m, 3H), 5.05 (s, 1H), 3.77-3.88 (m, 4H), 3.24-3.31 (m, 4H); MS (ESI) m/z: 414.0 [M+H⁺].

4-(4-(2-Ethynylthiazole-4-carbonyl)piperazin-1-yl)benzonitrile D26. ¹H NMR (400 MHz, DMSO-d₆) δ 8.29 (s, 1H), 7.61 (d, J=9.0 Hz, 2H), 7.03 (d, J=9.1 Hz, 2H), 5.03 (s, 1H), 3.79 (d, J=18.3 Hz, 4H), 3.44 (d, J=20.5 Hz, 4H); MS (ESI) m/z: 323.1 [M+H⁺].

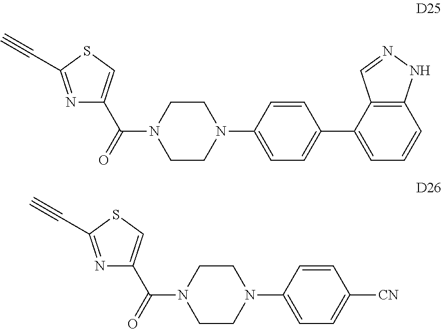 [SMILES: C#Cc1nc(C(=O)N2CCN(c3ccc(-c4cccc5[nH]ncc45)cc3)CC2)cs1 ; C#Cc1nc(C(=O)N2CCN(c3ccc(C#N)cc3)CC2)cs1]

(2-Ethynylthiazol-4-yl)(4-(4-nitrophenyl)piperazin-1-yl)methanone D27. ¹H NMR (400 MHz, DMSO-d₆) δ 8.30 (s, 1H), 8.08 (d, J=9.4 Hz, 2H), 7.03 (d, J=9.5 Hz, 2H), 5.04 (s, 1H), 3.82 (d, J=28.9 Hz, 4H), 3.58 (d, J=22.9 Hz, 4H); MS (ESI) m/z: 343.1 [M+H⁺].

(2-Ethynylthiazol-4-yl)(4-phenylpiperazin-1-yl)methanone D28. ¹H NMR (400 MHz, DMSO-d₆) δ 8.27 (s, 1H), 7.27-7.18 (m, 2H), 7.00-6.93 (m, 2H), 6.81 (t, J=7.3 Hz, 1H), 5.03 (s, 1H), 3.78 (s, 4H), 3.19 (d, J=18.7 Hz, 4H); MS (ESI) m/z: 298.1 [M+H⁺].

(2-Ethynylthiazol-4-yl)(4-phenylpiperidin-1-yl)methanone D29. ¹H NMR (400 MHz, DMSO-d₆) δ 8.21 (s, 1H), 7.34-7.24 (m, 4H), 7.20 (dd, J=11.2, 4.3 Hz, 1H), 5.01 (s, 1H), 4.62 (d, J=12.1 Hz, 1H), 4.12 (d, J=12.6 Hz, 1H), 3.20 (t, J=11.9 Hz, 1H), 2.84 (dd, J=9.6, 6.1 Hz, 2H), 1.83 (dd, J=37.3, 11.7 Hz, 2H), 1.60 (d, J=9.8 Hz, 2H); MS (ESI) m/z: 297.1 [M+H⁺].

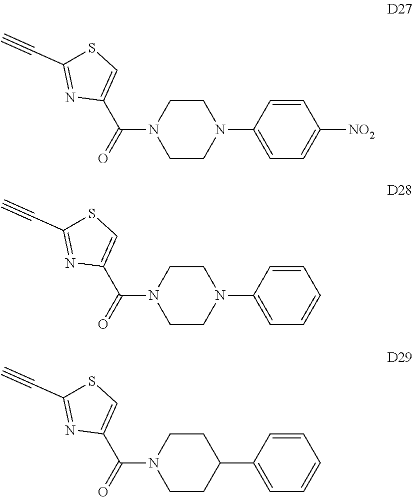 [SMILES: C#Cc1nc(C(=O)N2CCN(c3ccc([N+](=O)[O-])cc3)CC2)cs1 ; C#Cc1nc(C(=O)N2CCN(c3ccccc3)CC2)cs1 ; C#Cc1nc(C(=O)N2CCC(c3ccccc3)CC2)cs1]

(3,4-Dihydroisoquinolin-2(1H)-yl)(2-ethynylthiazol-4-yl)methanone D30. ¹H NMR (400 MHz, DMSO-d₆) δ 8.27 (d, J=4.8 Hz, 1H), 7.30-7.04 (m, 4H), 5.03 (s, 1H), 4.79 (d, J=9.6 Hz, 2H), 3.82 (d, J=5.5 Hz, 2H), 2.89 (t, J=5.9 Hz, 2H); MS (ESI) m/z: 269.1 [M+H⁺].

(2-Ethynylthiazol-4-yl)(4-hydroxypiperidin-1-yl)methanone D31. ¹H NMR (400 MHz, DMSO-d₆) δ 8.18 (s, 1H), 5.01 (s, 1H), 3.94-4.05 (m, 1H), 3.62-3.86 (m, 3H), 3.17-3.32 (m, 2H), 1.68-1.85 (m, 2H), 1.29-1.42 (m, 2H); MS (ESI) m/z: 237.0 [M+H⁺].

(2-Ethynylthiazol-4-yl)(2-(2-hydroxyethyl)piperidin-1-yl)methanone D32. ¹H NMR (400 MHz, DMSO-d₆) δ 8.11 (s, 1H), 5.00 (s, 1H), 4.49 (t, J=87.8 Hz, 2H), 3.89 (d, J=180.4 Hz, 1H), 3.41 (s, 1H), 2.95 (d, J=122.5 Hz, 1H), 1.90 (dq, J=8.4, 6.4 Hz, 1H), 1.62 (d, J=30.4 Hz, 7H), 1.44-1.29 (m, 1H); MS (ESI) m/z: 265.1 [M+H⁺].

(2-Ethynylthiazol-4-yl)(3-(hydroxymethyl)piperidin-1-yl)methanone D33. ¹H NMR (400 MHz, DMSO-d₆) δ 8.14 (d, J=7.7 Hz, 1H), 5.00 (s, 1H), 4.35 (dd, J=72.8, 12.1 Hz, 1H), 3.86 (d, J=15.7 Hz, 1H), 3.34 (d, J=4.9 Hz, 1H), 3.31-3.22 (m, 1H), 3.17 (s, 1H), 2.86 (d, J=11.5 Hz, 1H), 2.63-2.51 (m, 1H), 1.79-1.50 (m, 3H), 1.41 (d, J=12.7 Hz, 1H), 1.24 (s, 1H); MS (ESI) m/z: 251.1 [M+H⁺].

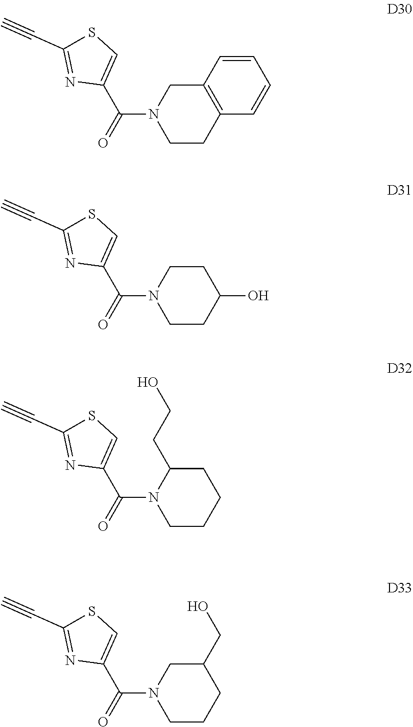 [SMILES: C#Cc1nc(C(=O)N2CCc3ccccc3C2)cs1 ; C#Cc1nc(C(=O)N2CCC(O)CC2)cs1 ; C#Cc1nc(C(=O)N2CCCCC2CCO)cs1 ; C#Cc1nc(C(=O)N2CCCC(CO)C2)cs1]

(2-Ethynylthiazol-4-yl)(3-hydroxypiperidin-1-yl)methanone D34. ¹H NMR (400 MHz, DMSO-d₆) δ 8.15 (s, 1H), 5.00 (s, 1H), 4.17 (d, J=11.5 Hz, 0.5H), 3.84-3.64 (m, 2H), 3.55-3.46 (m, 1H), 3.30-3.02 (m, 2H), 2.83 (m, 0.5H), 1.95-1.59 (m, 2H), 1.42 (dd, J=18.0, 9.4 Hz, 2H); MS (ESI) m/z: 237.1 [M+H⁺].

Methyl 1-(2-ethynylthiazole-4-carbonyl)piperidine-4-carboxylate D35. ¹H NMR (400 MHz, DMSO-d₆) δ 8.20 (s, 1H), 5.02 (s, 1H), 4.25-4.40 (m, 1H), 3.85-4.05 (m, 1H), 3.63 (s, 3H), 3.11-3.27 (m, 1H), 2.88-3.04 (m, 1H), 2.64-2.76 (m, 1H), 1.78-2.01 (m, 2H), 1.45-1.61 (m, 2H); MS (ESI) m/z: 279.1 [M+H⁺].

Methyl 1-(2-ethynylthiazole-4-carbonyl)piperidine-3-carboxylate D36. ¹H NMR (400 MHz, DMSO-d₆) δ 8.19 (s, 1H), 5.01 (s, 1H), 4.44-3.88 (m, 2H), 3.59 (d, J=43.8 Hz, 4H), 3.25-3.03 (m, 1H), 2.59 (s, 1H), 1.98 (s, 1H), 1.74-1.60 (m, 2H), 1.48 (s, 1H); MS (ESI) m/z: 279.1 [M+H⁺].

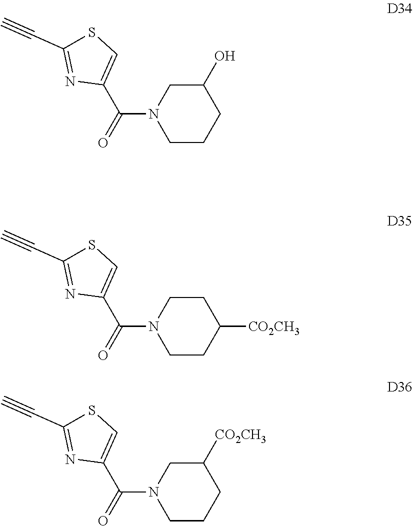 [SMILES: C#Cc1nc(C(=O)N2CCCC(O)C2)cs1 ; C#Cc1nc(C(=O)N2CCC(C(=O)OC)CC2)cs1 ; C#Cc1nc(C(=O)N2CCCC(C(=O)OC)C2)cs1]

Methyl 1-(2-ethynylthiazole-4-carbonyl)piperidine-2-carboxylate D37. ¹H NMR (400 MHz, DMSO-d₆) δ 8.24 (s, 1H), 5.23 (s, 1H), 5.02 (d, J=7.3 Hz, 1H), 4.40-4.03 (m, 1H), 3.69 (d, J=14.8 Hz, 3H), 3.24-2.75 (m, 1H), 2.15 (dd, J=22.1, 14.0 Hz, 1H), 1.78-1.56 (m, 3H), 1.52-1.20 (m, 2H); MS (ESI) m/z: 279.1 [M+H⁺].

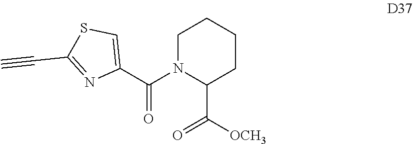 [SMILES: C#Cc1nc(C(=O)N2CCCCC2C(=O)OC)cs1]

### Example 3

### Preparation of 3-(4-(benzo[d]thiazol-7-yl)phenyl)-N-(2-ethynylthiazol-4-yl)propanamide E1

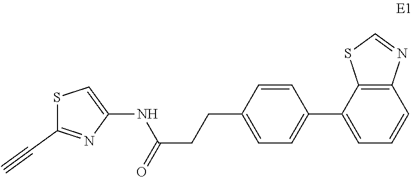 [SMILES: C#Cc1nc(NC(=O)CCc2ccc(-c3cccc4ncsc34)cc2)cs1]

Compound E1 was synthesized as shown in Scheme 4.

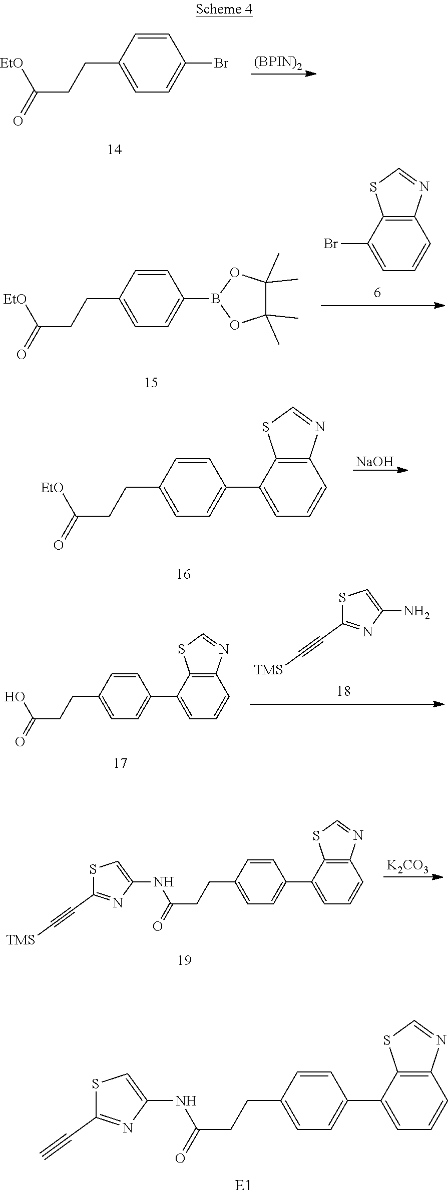

Ethyl 3-(4-(4,4,5,5-tetramethyl-1,3,2-dioxaborolan-2-yl)phenyl)propanoate 15. A mixture of compound 14 (1.0 g, 3.89 mmol), Pd(dppf)Cl₂ (300.0 mg, 0.39 mmol), (BPIN)₂ (1.2 g, 4.67 mmol), and KOAc (1.1 g, 11.67 mmol) in dry 1,4-dioxane (25 mL) was stirred under nitrogen at 90° C. for 3 h. The reaction mixture was then concentrated and purified by flash chromatography (PE) to yield compound 15 (500 mg) in 42% yield. LCMS (ESI) m/z: 305.2 [M+H⁺].

Ethyl 3-(4-(benzothiazol-7-yl)phenyl)propanoate 16. A mixture of compound 6 (288 mg, 1.35 mmol), compound 15 (450 mg, 1.48 mmol), Pd(dppf)Cl₂ (50 mg, 0.07 mmol), and Cs₂CO₃ (875 mg, 2.7 mmol) in dioxane (8 mL) and H₂O (2 mL) was stirred under nitrogen at 90° C. for 4 h. The reaction mixture was then concentrated and purified by flash chromatography eluted (PE/EA: 100/0 to 90/10) to yield compound 16 (250 mg) in 53% yield. LCMS (ESI) nm/z: 312.1 [M+H⁺].

3-(4-(Benzothiazol-7-yl)phenyl)propanoic acid 17. A mixture of compound 16 (250 mg, 0.8 mmol) and NaOH (40 mg, 1.0 mmol) in THF (8 mL) and H₂O (2 mL) was stirred at 60° C. for 3 h. The reaction mixture was then concentrated, neutralized with HCl (2 M in H₂O) to pH<3, and extracted with DCM (10 mL×3). The organic layers were combined, washed with brine (10 mL×2), dried over anhydrous Na₂SO₄, and concentrated to yield compound 17 (138.8 mg) in 61% yield. LCMS (ESI) m/z: 284.1 [M+H⁺].

3-(4-(Benzothiazol-7-yl)phenyl)-N-(2-((trimethylsilyl)ethynyl)thiazol-4-yl)propenamide 19. A mixture of compound 17 (40 mg, 0.14 mmol), compound 18 (26.4 mg, 0.13 mmol), HATU (61.1 mg, 0.16 mmol), and DIPEA (34.6 mg, 0.26 mmol) in DMF (3 mL), was stirred at room temperature for 1 h. The reaction mixture was then concentrated and purified by flash chromatography (PE/EA: 100/0 to 85/15) to yield compound 19 (53 mg) in 87% yield. LCMS (ESI) m/z: 462.1 [M+H⁺].

3-(4-(Benzo[d]thiazol-7-yl)phenyl)-N-(2-ethynylthiazol-4-yl)propanamide E1. 3-(4-(Benzothiazol-7-yl)phenyl)-N-(2-ethynylthiazol-4-yl)propenamide E1. A mixture of compound 19 (53 mg, 0.11 mmol) and K₂CO₃ (16 mg, 0.11 mmol) in MeOH (3 mL) was stirred at room temperature for 1 h. The reaction was then concentrated and purified by prep-HPLC to yield compound E1 (15.8 mg) in 37% yield. ¹H NMR (400 MHz, DMSO-d₆) δ 11.16 (s, 1H), 9.44 (s, 1H), 8.09 (dd, J=8.0, 1.0 Hz, 1H), 7.72 (s, 1H), 7.65 (dd, J=8.0, 1.4 Hz, 3H), 7.57 (d, J=6.8 Hz, 1H), 7.43 (d, J=8.2 Hz, 2H), 4.93 (s, 1H), 3.00 (t, J=7.6 Hz, 2H), 2.75 (t, J=7.7 Hz, 2H); MS (ESI) m/z: 390.1 [M+H⁺].

Compounds E2 to E12 were prepared.

(S)-2-Amino-N-(2-ethynylthiazol-4-yl)-3-phenylpropanamide E2. ¹H NMR (400 MHz, DMSO-d₆) δ 8.21 (s, 1H), 7.72 (s, 1H), 7.26 (dd, J=8.7, 4.2 Hz, 5H), 4.94 (s, 1H), 3.72 (dd, J=8.0, 5.7 Hz, 1H), 3.02 (dd, J=13.4, 5.6 Hz, 1H), 2.75 (dd, J=13.4, 8.1 Hz, 1H); MS (ESI) m/z: 272.1 [M+H⁺].

(R)-2-Amino-N-(2-ethynylthiazol-4-yl)-3-phenylpropanamide E3. ¹H NMR (400 MHz, DMSO-d₆) δ 7.71 (s, 1H), 7.29-7.16 (m, 6H), 4.94 (s, 1H), 3.62 (dt, J=26.6, 13.3 Hz, 2H), 2.99 (dd, J=13.4, 5.5 Hz, 1H), 2.69 (dd, J=13.4, 8.1 Hz, 1H); MS (ESI) m/z: 272.0 [M+H⁺].

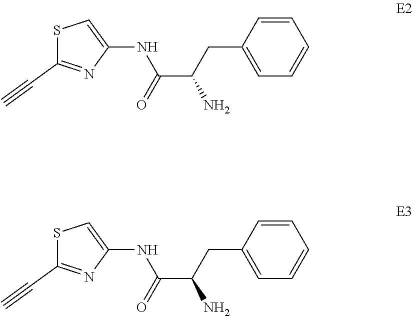 [SMILES: C#Cc1nc(NC(=O)[C@@H](N)Cc2ccccc2)cs1 ; C#Cc1nc(NC(=O)[C@H](N)Cc2ccccc2)cs1]

N-(2-Ethynylthiazol-4-yl)-3-(4-(1-methyl-1H-indazol-4-yl)phenyl)propanamide E4. ¹H NMR (400 MHz, DMSO-d₆) δ 11.16 (s, 1H), 8.12 (d, J=0.8 Hz, 1H), 7.72 (s, 1H), 7.64 (dd, J=12.4, 8.3 Hz, 3H), 7.47 (dd, J=8.4, 7.1 Hz, 1H), 7.40 (d, J=8.1 Hz, 2H), 7.24 (d, J=6.8 Hz, 1H), 4.93 (s, 1H), 4.08 (s, 3H), 2.99 (t, J=7.6 Hz, 2H), 2.74 (t, J=7.7 Hz, 2H); MS (ESI) m/z: 386.5 [M+H⁺].

(R)-2-Amino-3-(2-cyanophenyl)-N-(2-ethynylthiazol-4-yl)propenamide E5. ¹H NMR (400 MHz, DMSO-d₆) δ 7.76 (dd, J=7.7, 1.1 Hz, 1H), 7.72 (s, 1H), 7.62 (td, J=7.7, 1.3 Hz, 1H), 7.52 (d, J=7.4 Hz, 1H), 7.40 (td, J=7.6, 1.1 Hz, 1H), 4.94 (s, 1H), 3.71 (dd, J=8.5, 5.7 Hz, 1H), 3.16 (dd, J=13.7, 5.7 Hz, 1H), 2.91 (dd, J=13.6, 8.6 Hz, 1H); MS (ESI) m/z: 297.1 [M+H⁺].

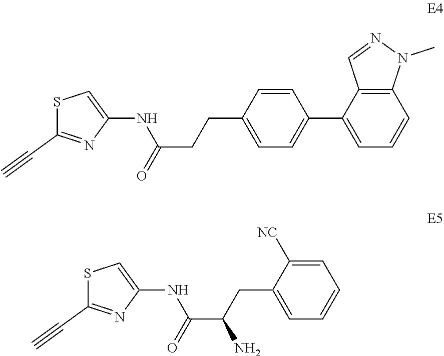

2-Ethynyl-N-(3-(thiazol-5-yl)phenethyl)thiazole-4-carboxamide E6. ¹H NMR (400 MHz, DMSO-d₆) δ 9.07 (s, 1H), 8.79-8.58 (m, 1H), 8.34 (s, 1H), 8.27 (s, 1H), 7.62-7.48 (m, 2H), 7.46-7.32 (m, 1H), 7.23 (d, J=7.6 Hz, 1H), 5.03 (s, 1H), 3.54 (dd, J=13.8, 6.7 Hz, 1H), 2.91 (t, J=7.2 Hz, 2H); MS (ESI) m/z: 340.1 [M+H⁺].

2-Ethynyl-N-(3-(thiazol-4-yl)phenethyl)thiazole-4-carboxamide E7. ¹H NMR (400 MHz, DMSO-d₆) δ 9.18 (d, J=1.9 Hz, 1H), 8.67 (t, J=5.8 Hz, 1H), 8.35 (s, 1H), 8.13 (d, J=1.9 Hz, 1H), 7.88 (s, 1H), 7.82 (d, J=7.8 Hz, 1H), 7.37 (t, J=7.6 Hz, 1H), 7.22 (d, J=7.7 Hz, 1H), 5.04 (s, 1H), 3.54 (dd, J=14.5, 6.4 Hz, 2H), 2.91 (t, J=7.4 Hz, 2H); MS (ESI) m/z: 340.0 [M+H⁺].

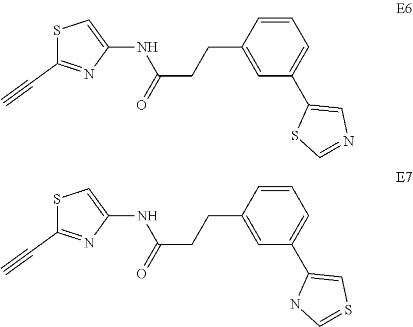

(S)-2-Amino-3-(2-cyanophenyl)-N-(2-ethynylthiazol-4-yl)propenamide E8. ¹H NMR (400 MHz, DMSO-d₆) δ 7.76 (dd, J=7.7, 1.1 Hz, 1H), 7.72 (s, 1H), 7.62 (td, J=7.7, 1.3 Hz, 1H), 7.52 (d, J=7.7 Hz, 1H), 7.40 (td, J=7.6, 1.1 Hz, 1H), 4.94 (s, 1H), 3.71 (dd, J=8.5, 5.7 Hz, 1H), 3.16 (dd, J=13.7, 5.7 Hz, 1H), 2.91 (dd, J=13.7, 8.6 Hz, 1H); MS (ESI) m/z: 297.1 [M+H⁺].

3-(Benzo[d][1,3]dioxol-5-yl)-N-(2-ethynylthiazol-4-yl)propanamide E9. ¹H NMR (400 MHz, DMSO-d₆) δ 11.07 (s, 1H), 7.68 (s, 1H), 6.81 (dd, J=6.7, 4.8 Hz, 2H), 6.68 (dd, J=7.9, 1.7 Hz, 1H), 5.95 (s, 2H), 4.92 (s, 1H), 2.81 (t, J=7.6 Hz, 2H), 2.61 (t, J=7.7 Hz, 2H); MS (ESI) m/z: 301.1 [M+H⁺].

N-(2-Ethynylthiazol-4-yl)quinoline-2-carboxamide E10. ¹H NMR (400 MHz, DMSO-d₆) δ 11.22 (s, 1H), 8.67 (d, J=8.5 Hz, 1H), 8.26 (d, J=8.4 Hz, 2H), 8.14 (d, J=8.1 Hz, 1H), 7.99 (s, 1H), 7.93 (t, J=7.3 Hz, 1H), 7.78 (t, J=7.4 Hz, 1H), 5.03 (s, 1H); MS (ESI) m/z: 280.0 [M+H⁺].

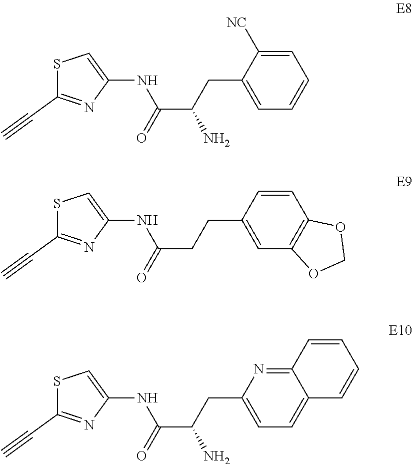 [SMILES: C#Cc1nc(NC(=O)[C@@H](N)Cc2ccccc2C#N)cs1 ; C#Cc1nc(NC(=O)CCc2ccc3c(c2)OCO3)cs1 ; C#Cc1nc(NC(=O)[C@@H](N)Cc2ccc3ccccc3n2)cs1]

N-(2-Ethynylthiazol-4-yl)cinnamamide E11. ¹H NMR (400 MHz, DMSO-d₆) δ 8.58 (brs, 1H), 7.87 (s, 1H), 7.81 (d, J=15.6 Hz, 1H), 7.56-7.62 (m, 2H), 7.40-7.47 (m, 3H), 6.56 (d, J=15.6 Hz, 2H); MS (ESI) m/z: 255.1 [M+H⁺].

N-(2-Ethynylthiazol-4-yl)benzamide E12. ¹H NMR (400 MHz, DMSO-d₆) δ 11.54 (s, 1H), 8.03 (d, J=7.2 Hz, 2H), 7.93 (s, 1H), 7.60 (t, J=7.3 Hz, 1H), 7.52 (t, J=7.5 Hz, 2H), 4.97 (s, 1H); MS (ESI) m/z: 229.1 [M+H⁺].

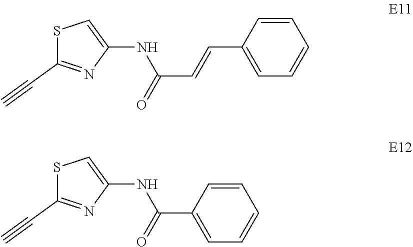 [SMILES: C#Cc1nc(NC(=O)/C=C/c2ccccc2)cs1 ; C#Cc1nc(NC(=O)c2ccccc2)cs1]

### Example B1

### Cell Proliferation Assay

The activity of a compound was determined in a cell proliferation assay using the hepatocellular carcinoma cell line Hub-7. Cells (4,000 cells per well) were seeded in a 96-well tissue culture plate. After incubated overnight, the compound was added at predetermined concentrations, starting with 10 μM and followed by 3-fold serial dilution. After the cells were incubated for 24 h, cell viability was determined using a CELL-TITER GLO® assay. The CELL-TITER GLO® reagent (50 μL) was added to each well and the luminescence was measured by a multimode microplate reader after a brief period of shaking. EC₅₀ values were determined for the compound. The results are summarized in Table 1, wherein A represents a value no greater than 200 nM, B represents a value greater than 200 nM but no greater than 1 μM, C represents a value greater than 1 μM but no greater than 5 μM, and D represents a value greater than 5 μM.

TABLE 1
Inhibition of Hub-7 Cell Proliferation

| Cmpd. | EC₅₀ | Cmpd. | EC₅₀ | Cmpd. | EC₅₀ | Cmpd. | EC₅₀ |
|---|---|---|---|---|---|---|---|
| A1 | B | C28 | C | C84 | C | C140 | C |
| A2 | C | C29 | B | C85 | C | C141 | C |
| A3 | C | C30 | C | C86 | C | C142 | D |
| A4 | B | C31 | B | C87 | B | C143 | B |
| A5 | B | C32 | B | C88 | C | C144 | C |
| A6 | A | C33 | B | C89 | D | C145 | D |
| A7 | A | C34 | A | C90 | D | C148 | C |
| A8 | A | C35 | B | C91 | C | D1 | A |
| A9 | A | C36 | B | C92 | C | D2 | A |
| A10 | B | C37 | C | C93 | B | D3 | B |
| A11 | B | C38 | C | C94 | C | D4 | A |
| A12 | B | C39 | B | C95 | C | D5 | C |
| A13 | B | C40 | C | C96 | C | D6 | A |
| A14 | B | C41 | C | C97 | B | D7 | A |
| A15 | A | C42 | B | C98 | D | D8 | B |
| A16 | A | C43 | B | C99 | C | D9 | A |
| A17 | C | C44 | A | C100 | B | D10 | A |
| A18 | B | C45 | C | C101 | D | D11 | A |
| A19 | B | C46 | B | C102 | B | D12 | A |
| A20 | C | C47 | C | C103 | A | D13 | A |
| A21 | B | C48 | B | C104 | B | D14 | A |
| A22 | A | C49 | A | C105 | C | D15 | B |
| A23 | B | C50 | A | C106 | B | D16 | B |
| B1 | B | C51 | B | C107 | B | D17 | A |
| B2 | A | C52 | B | C108 | C | D18 | A |
| B3 | B | C53 | B | C109 | C | D19 | A |
| B4 | B | C54 | B | C110 | B | D20 | A |
| B5 | A | C55 | B | C111 | D | D21 | A |
| B6 | A | C56 | C | C112 | B | D22 | A |
| C1 | B | C57 | B | C113 | A | D23 | A |
| C2 | A | C58 | C | C114 | A | D24 | A |
| C3 | B | C59 | C | C115 | A | D25 | A |
| C4 | A | C60 | B | C116 | C | D26 | B |
| C5 | A | C61 | B | C117 | B | D27 | A |
| C6 | A | C62 | B | C118 | B | D28 | B |
| C7 | A | C63 | A | C119 | D | D29 | B |
| C8 | B | C64 | A | C120 | D | D30 | B |
| C9 | B | C65 | C | C121 | C | D31 | D |
| C10 | A | C66 | C | C122 | C | D32 | D |
| C11 | B | C67 | C | C123 | B | D33 | D |
| C12 | C | C68 | B | C124 | B | D34 | D |
| C13 | B | C69 | A | C125 | A | D35 | D |
| C14 | B | C70 | C | C126 | C | D36 | D |
| C15 | B | C71 | B | C127 | B | D37 | D |
| C16 | B | C72 | B | C128 | C | E1 | B |
| C17 | A | C73 | D | C129 | A | E2 | C |
| C18 | B | C74 | B | C130 | C | E3 | B |
| C19 | B | C75 | B | C131 | C | E4 | A |
| C20 | B | C76 | C | C132 | D | E5 | A |
| C21 | A | C77 | B | C133 | C | E6 | B |
| C22 | B | C78 | B | C134 | C | E7 | C |
| C23 | B | C79 | B | C135 | C | E8 | C |
| C24 | A | C80 | B | C136 | D | E9 | B |
| C25 | B | C81 | C | C137 | C | E10 | C |
| C26 | C | C82 | C | C138 | C | E11 | B |
| C27 | B | C83 | C | C139 | D | E12 | C |

### Example B2

### Cell Proliferation Assay

The activity of a compound was determined in a cell proliferation assay using the lung cancer cell line NCI-H82. Cells (1,000 cells per well) were cultured with RPMI1640 in a 384-well tissue culture plate. After incubated overnight, the compound was added at predetermined concentrations, starting with 1 μM and followed by a 3-fold serial dilution. After the cells were incubated for 72 h, cell viability was determined using a CELL-TITER GLO assay. The CELL-TITER GLO reagent (25 μL) was added to each well and the luminescence was measured by a multimode microplate reader after a brief period of shaking. EC₅₀ values were determined for the compound. The results are summarized in Table 1, wherein A represents a value no greater than 200 nM, B represents a value greater than 200 nM but no greater than 1 μM, C represents a value greater than 1 μM but no greater than 5 NM, and D represents a value greater than 5 μM.

TABLE 2
Inhibition of NCl-H82 Cell Proliferation

| Cmpd. | EC₅₀ | Cmpd. | EC₅₀ | Cmpd. | EC₅₀ | Cmpd. | EC₅₀ |
|---|---|---|---|---|---|---|---|
| A25 | A | A84 | A | A142 | A | A199 | A |
| A26 | B | A85 | A | A143 | A | A200 | A |
| A29 | A | A86 | A | A144 | B | A201 | A |
| A30 | A | A87 | A | A145 | B | A202 | A |
| A31 | B | A88 | A | A146 | B | A203 | A |
| A32 | A | A89 | A | A147 | B | A204 | A |
| A33 | A | A90 | A | A148 | A | A205 | A |
| A34 | B | A91 | A | A149 | A | A206 | A |
| A35 | B | A92 | A | A150 | A | A207 | A |
| A36 | A | A93 | A | A151 | A | A208 | A |
| A37 | A | A94 | A | A152 | A | A209 | A |
| A38 | A | A95 | A | A153 | A | A210 | A |
| A40 | A | A96 | A | A154 | A | A211 | A |
| A41 | A | A97 | A | A155 | A | A212 | A |
| A42 | A | A98 | A | A156 | A | A213 | A |
| A43 | A | A99 | A | A157 | A | A214 | A |
| A44 | A | A100 | A | A158 | A | A215 | A |
| A45 | A | A101 | A | A159 | D | A216 | A |
| A46 | A | A102 | A | A160 | B | A217 | A |
| A47 | A | A103 | A | A161 | A | A218 | A |
| A48 | A | A104 | A | A162 | A | A219 | A |
| A49 | A | A105 | A | A163 | A | A220 | A |
| A50 | A | A107 | A | A165 | A | A221 | A |
| A51 | A | A108 | B | A166 | A | A222 | A |
| A52 | A | A109 | A | A167 | A | A223 | A |
| A53 | A | A110 | A | A168 | A | A224 | A |
| A54 | A | A111 | A | A169 | A | A225 | A |
| A55 | A | A112 | A | A170 | A | A226 | A |
| A56 | A | A113 | B | A171 | A | A227 | A |
| A57 | A | A114 | A | A172 | A | A228 | A |
| A58 | A | A115 | A | A173 | B | A229 | A |
| A59 | A | A116 | A | A174 | A | A230 | A |
| A60 | A | A117 | A | A175 | A | A231 | A |
| A61 | A | A118 | B | A176 | A | A232 | A |
| A62 | A | A119 | A | A177 | B | A233 | A |
| A63 | A | A120 | A | A178 | A | A234 | A |
| A64 | A | A122 | B | A179 | A | A235 | A |
| A65 | A | A123 | A | A180 | A | A236 | A |
| A66 | A | A124 | B | A181 | A | A238 | A |
| A67 | A | A125 | A | A182 | A | A239 | A |
| A68 | A | A126 | A | A183 | A | A240 | A |
| A69 | A | A127 | A | A184 | B | A241 | A |
| A70 | A | A128 | A | A185 | B | B15 | A |
| A71 | A | A129 | A | A186 | A | B18 | A |
| A72 | A | A130 | A | A187 | A | B23 | A |
| A73 | A | A131 | A | A188 | A | B24 | A |
| A74 | A | A132 | A | A189 | A | B25 | A |
| A75 | A | A133 | A | A190 | A | B27 | A |
| A76 | A | A134 | B | A191 | A | B28 | A |
| A77 | A | A135 | A | A192 | B | B52 | A |
| A78 | A | A136 | A | A193 | B | B53 | A |
| A79 | A | A137 | A | A194 | B | B54 | A |
| A80 | A | A138 | A | A195 | A | B56 | A |
| A81 | A | A139 | A | A196 | A | B59 | A |
| A82 | A | A140 | A | A197 | A | | |
| A83 | A | A141 | A | A198 | A | | |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A compound of Formula (XLII) or (I): 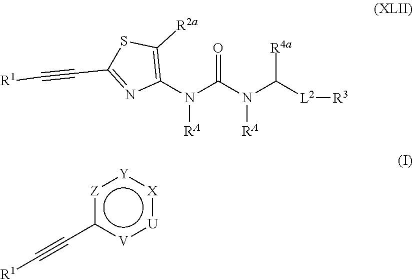
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R^{A} in Formula (XLII) is independently hydrogen or C₁₋₆ alkyl;
R^{4a} in Formula (XLII) is C₁₋₆ alkyl or —C(O)NR^{1b}R^{1c};
U, V, X, and Z are each independently —C(R^{2a})═, —N═, —N(R^{2b})—, —O—, —S—, or -A-L¹-L²-R³; with the proviso that at least one of U and Z is —N═, and one of U, V, X, and Z is -A-L¹-L²-R³;
Y is a bond, —C(R^{2a})═, or —N═;
A is —C(O)—, —C(O)NR^{1a}—, —OC(O)NR^{1a}—, —NR^{1a}C(O)NR^{1d}—, —S(O)—, —S(O)₂—, —S(O)NR^{1a}—, or —S(O)₂NR^{1a}—;
L¹ is a bond, C₁₋₆ alkylene, C₂₋₆ alkenylene, C₂₋₆ alkynylene, C₃₋₁₀ cycloalkylene, C₆₋₁₄ arylene, heteroarylene, or heterocyclylene;
L² is C₆₋₁₄ arylene, heteroarylene, or heterocyclylene;
R¹ is hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₈ aralkyl, heteroaryl, or heterocyclyl;
R³ is (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2a} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{2b} is independently (i) hydrogen, deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; and
each R^{1a}, R^{1b}, R^{1c}, and R^{1d} is independently hydrogen, deuterium, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl;
wherein each alkyl, alkylene, alkenyl, alkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, wherein each Q is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(O)SR^{a}, —C(NR^{a})NR^{b}R^{c}, —C(S)R^{a}, —C(S)OR^{a}, —C(S)NR^{b}R^{c}, —OR^{a}, —OC(O)R^{a}, —OC(O)OR^{a}, —OC(O)NR^{b}R^{c}, —OC(O)SR^{a}, —OC(NR^{a})NR^{b}R^{c}, —OC(S)R^{a}, —OC(S)OR^{a}, —OC(S)NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(O)SR^{d}, —NR^{a}C(NR^{d})NR^{b}R^{c}, —NR^{a}C(S)R^{d}, —NR^{a}C(S)OR^{d}, —NR^{a}C(S)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl optionally substituted with one or more, in one embodiment, one, two, three, or four, substituents Q^{a};
wherein each Q^{a} is independently selected from: (a) deuterium, cyano, halo, nitro, and oxo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(O)SR^{e}, —C(NR^{e})NR^{f}R^{g}, —C(S)R^{e}, —C(S)OR^{e}, —C(S)NR^{f}R^{g}, —OR^{e}, —OC(O)R^{e}, —OC(O)OR^{e}, —OC(O)NR^{f}R^{g}, —OC(O)SR^{e}, —OC(NR^{e})NR^{f}R^{g}, —OC(S)R^{e}, —OC(S)OR^{e}, —OC(S)NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{f}, —NR^{e}C(O)NR^{f}R^{g}, —NR^{e}C(O)SR^{f}, —NR^{e}C(NR^{h})NR^{f}R^{g}, —NR^{e}C(S)R^{h}, —NR^{e}C(S)OR^{f}, —NRC(S)NR^{f}R^{g}, —NR^{e}S(O)R^{h}, —NRS(O)₂R^{h}, —NRS(O)NR^{f}R^{g}, —NRS(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen or deuterium; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

2. The compound of claim 1, wherein the compound is a compound of Formula (I): 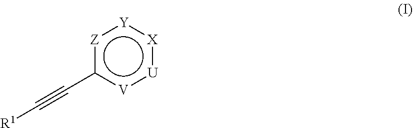
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

3. The compound of claim 1 or 2, wherein U is -A-L¹-L²-R³.

4. The compound of claim 1 or 2, wherein V is -A-L¹-L²-R³.

5. The compound of claim 1 or 2, wherein X is -A-Lt-L²-R³.

6. The compound of claim 1, 2, or 4, wherein the compound is a compound of Formula (III): 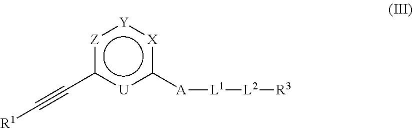
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

7. The compound of any one of claims 1, 2, and 4 to 6, wherein U is —N═.

8. The compound of any one of claims 1, 2, and 4 to 6, wherein U is —S—.

9. The compound of any one of claims 1, 2, and 4 to 6, wherein U is —O—.

10. The compound of any one of claims 1, 2, and 4 to 6, wherein U is —N(R^{2b})—.

11. The compound of claim 10, wherein U is —N(H)— or —N(CH₃)—.

12. The compound of any one of claims 1 to 4 and 6 to 11, wherein X is —N═.

13. The compound of any one of claims 1 to 4 and 6 to 11, wherein X is —C(R^{a})═.

14. The compound of claim 13, wherein X is —C(H)═, —C(CH₃)═, or —C(phenyl)═.

15. The compound of any one of claims 1 to 4 and 6 to 11, wherein X is —S—.

16. The compound of any one of claims 1 to 4 and 6 to 11, wherein X is —O—.

17. The compound of any one of claims 1 to 4 and 6 to 11, wherein X is —N(R^{2b}).

18. The compound of claim 17, wherein X is —N(H)— or —N(CH₃)—.

19. The compound of any one of claims 1 to 18, wherein Y is a bond.

20. The compound of any one of claims 1 to 18, wherein Y is —C(R^{2a})═ or —N═.

21. The compound of claim 20, wherein Y is —C(H)═ or —N═.

22. The compound of any one of claims 1 to 21, wherein Z is —N═.

23. The compound of any one of claims 1 to 7 and 10 to 21, wherein Z is —S—.

24. The compound of any one of claims 1 to 7 and 10 to 21, wherein Z is —O—.

25. The compound of any one of claims 1 to 21, wherein Z is —N(R^{2b})—.

26. The compound of claim 25, wherein Z is —N(H)— or —N(CH₃)—.

27. The compound of claim 1, 2, or 6, wherein the compound is a compound of Formula (IX): 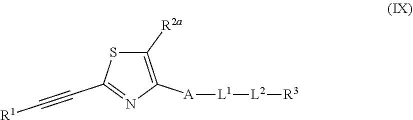
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

28. The compound of claim 1, 2, or 6, wherein the compound is a compound of Formula (XII): 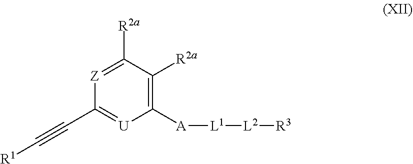
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein U is —N═ and Z is —C(R^{2a})═ or —N═; or U is —C(R^{2a})═ and Z is —N═.

29. The compound of claim 28, wherein U is —N═.

30. The compound of claim 28, wherein U is —C(R^{2a})═.

31. The compound of claim 30, wherein U is —C(H)═.

32. The compound of any one of claims 28 to 31, wherein Z is —N═.

33. The compound of claim 28 or 29, wherein Z is —C(R^{2a})═.

34. The compound of claim 33, wherein Z is —C(H)═.

35. The compound of any one of claims 1 to 34, wherein L¹ is a bond; or C₁₋₆ alkylene, C₂₋₆, C₃₋₁₀ cycloalkylene, or heterocyclylene, each of which is optionally substituted with one, two, or three substituents Q.

36. The compound of any one of claims 1 to 35, wherein L¹ is a bond.

37. The compound of any one of claims 1 to 35, wherein L¹ is C₁₋₆ alkylene, optionally substituted with one, two, or three substituents Q.

38. The compound of claim 37, wherein L¹ is methanediyl or ethanediyl, each of which is optionally substituted with amino, hydroxymethyl, or hydroxyl.

39. The compound of any one of claims 1 to 35, wherein L¹ is C₂₋₆ alkenylene, optionally substituted with one, two, or three substituents Q.

40. The compound of claim 39, wherein L¹ is ethenediyl.

41. The compound of any one of claims 1 to 35, wherein L¹ is C₃₋₁₀ cycloalkylene, optionally substituted with one, two, or three substituents Q.

42. The compound of claim 41, wherein L¹ is cyclopropanediyl.

43. The compound of any one of claims 1 to 35, wherein L¹ is heterocyclylene, optionally substituted with one, two, or three substituents Q.

44. The compound of claim 43, wherein L¹ is monocyclic heterocyclylene, optionally substituted with one, two, or three substituents Q.

45. The compound of claim 43 or 44, wherein L¹ is 3 to 7 membered heterocyclylene, each optionally substituted with one, two, or three substituents Q.

46. The compound of any one of claims 43 to 45, wherein L¹ is azetidindiyl, pyrrolidindiyl, piperidindiyl, or piperazindiyl, each of which is optionally substituted with fluoro, hydroxymethyl, hydroxyl, or amino.

47. The compound of any one of claims 1 to 35, wherein L¹ is a bond, methanediyl, ethane-1,1-diyl, 2-hydroxyethane-1,1-diyl, ethane-1,2-diyl, 1-hydroxyethane-1,2-diyl, 1-aminoethane-1,2-diyl, ethene-1,2-diyl, cyclopropan-1,1-diyl, azetidin-1,3-diyl, pyrrolidin-1,2-diyl, piperidin-1,4-diyl, 4-fluropiperidin-1,4-diyl, 4-hydroxypiperidin-1,4-diyl, piperazin-1,4-diyl, or 2-hydroxymethylpiperazin-1,4-diyl.

48. The compound of claim 1, wherein the compound is a compound of Formula 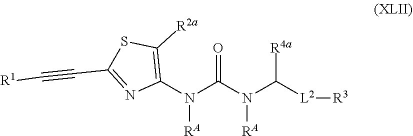
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

49. The compound of any one of claims 1 to 48, wherein L² is C₆₋₁₄ arylene, optionally substituted with one, two, or three substituents Q.

50. The compound of claim 49, wherein L² is monocyclic or bicyclic C₆₋₁₄ arylene, each optionally substituted with one, two, or three substituents Q.

51. The compound of claim 49 or 50, wherein L² is phendiyl, 2,3-dihydroindendiyl, or naphthdiyl, each optionally substituted with one, two, or three substituents Q.

52. The compound of any one of claims 49 to 51, wherein L² is phen-1,2-diyl, phen-1,3-diyl, phen-1,4-diyl, 2,3-dihydroinden-1,4-diyl, 2,3-dihydroinden-2,5-diyl, naphth-1,5-diyl, or naphth-2,6-diyl, each of which is optionally substituted with one or two substituents, where each substituent is independently cyano, fluoro, chloro, hydroxyl, or methoxy.

53. The compound of any one of claims 1 to 48, wherein L² is heteroarylene, optionally substituted with one, two, or three substituents Q.

54. The compound of claim 53, wherein L² is monocyclic or bicyclic heteroarylene, each optionally substituted with one, two, or three substituents Q.

55. The compound of claim 53 or 54, wherein L² is indoldiyl, indazoldiyl, benzothiazoldiyl, quinoldiyl, or quinoldiyl, each optionally substituted with one, two, or three substituents Q.

56. The compound of any one of claims 53 to 55, wherein L² is indol-2,5-diyl, indazol-3,7-diyl, benzothiazol-2,6-diyl, quinol-2,6-diyl, or quinol-3,7-diyl, each of which is optionally substituted with one or two substituents, where each substituent is independently cyano, fluoro, chloro, hydroxyl, or methoxy.

57. The compound of any one of claims 1 to 48, wherein L² is heterocyclylene, optionally substituted with one, two, or three substituents Q.

58. The compound of claim 57, wherein L² is monocyclic or bicyclic heterocyclylene, each optionally substituted with one, two, or three substituents Q.

59. The compound of claim 57 or 58, wherein L² is piperidindiyl, isoindolindiyl, 1,2,3,4-tetrahydroisoquinolindiyl, benzo[d][1,3]dioxoldiyl, or 2,3-dihydrobenzo[b][1,4]-dioxindiyl, each optionally substituted with one, two, or three substituents Q.

60. The compound of any one of claims 57 to 59, wherein L² is piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]dioxol-2,5-diyl, or 2,3-dihydrobenzo[b][1,4]dioxin-2,6-diyl, each of which is optionally substituted with one or two substituents, where each substituent is independently cyano, fluoro, chloro, hydroxyl, or methoxy.

61. The compound of any one of claims 1 to 48, wherein L² is phen-1,2-diyl, phen-1,3-diyl, phen-1,4-diyl, 4-methoxyphen-1,3-diyl, 2-cyanophen-1,4-diyl, 2-fluorophen-1,4-diyl, 2-chlorophen-1,4-diyl, 2-hydroxyphen-1,4-diyl, 2,3-dihydroinden-1,4-diyl, 2,3-dihydroinden-2,5-diyl, naphth-1,5-diyl, naphth-2,6-diyl, pyrazol-1,3-diyl, pyrazol-1,4-diyl, pyridin-2,3-diyl, pyridin-2,5-diyl, indol-2,5-diyl, indazol-3,7-diyl, benzothiazol-2,6-diyl, quinol-2,6-diyl, quinol-3,7-diyl, piperidin-1,2-diyl, piperidin-1,3-diyl, piperidin-1,4-diyl, isoindolin-2,5-diyl, 1,2,3,4-tetrahydroisoquinolin-2,6-diyl, benzo[d][1,3]-dioxol-2,5-diyl, or 2,3-dihydrobenzo[b][1,4]-dioxin-2,6-diyl.

62. The compound of claim 1 or 27, wherein the compound is a compound of Formula (XIII): 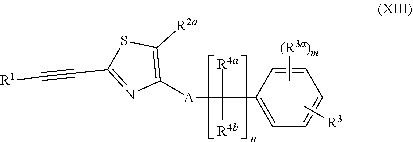
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R³, is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —CO(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{4a} and R^{4b} is independently (i) hydrogen, deuterium, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR¹²)NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or R^{4a} and R^{4b} together with the carbon atom to which they are attached form C₃₋₁₀ cycloalkyl;
m is an integer of 0, 1, 2, 3, or 4; and
n is an integer of 0, 1, 2, 3, 4, 5, or 6;
wherein each alkyl, alkenyl, alkynyl, cycloalkyl, aryl, aralkyl, heteroaryl, and heterocyclyl is optionally substituted with one or more, in one embodiment, one, two, three, or four, substituents Q.

63. The compound of claim 62, wherein the compound is a compound of Formula (XIV): 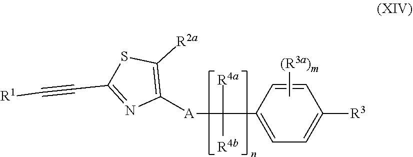
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

64. The compound of claim 1 or 27, wherein the compound is a compound of Formula (XXI): 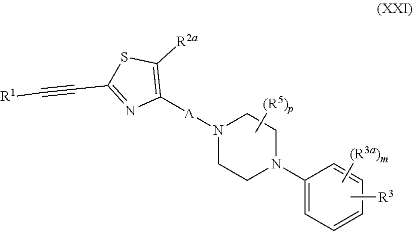
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R⁵ is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{3a} is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
m is an integer of 0, 1, 2, 3, or 4; and
p is an integer of 0, 1, 2, 3, or 4.

65. The compound of claim 64, wherein the compound is a compound of Formula 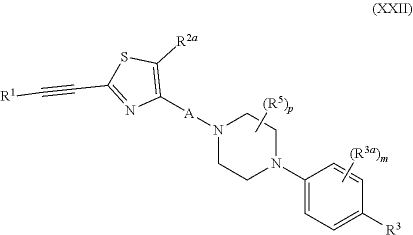
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

66. The compound of claim 1 or 28, wherein the compound is a compound of Formula (XXVII): 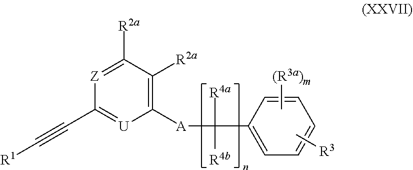
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R^{3a} is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —CO(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{4a} and R^{4b} is independently (i) hydrogen, deuterium, cyano, halo, or nitro;
(b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or R^{4a} and R^{4b} together with the carbon atom to which they are attached form C₃₋₁₀ cycloalkyl;
m is an integer of 0, 1, 2, 3, or 4; and
n is an integer of 0, 1, 2, 3, 4, 5, or 6;
wherein each alkyl, alkenyl, alkynyl, cycloalkyl, aryl, aralkyl, heteroaryl, and heterocyclyl is optionally substituted with one or more, in one embodiment, one, two, three, or four, substituents Q.

67. The compound of claim 66, wherein the compound is a compound of Formula (XXVIII): 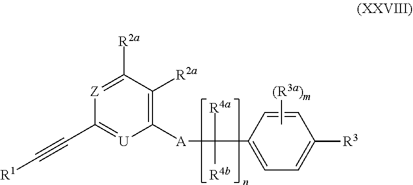
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

68. The compound of claim 1 or 28, wherein the compound is a compound of Formula (XXXV): 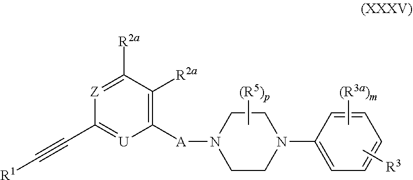
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein;
each R⁵ is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (iii) —C(O)R^{1a}, —C(O)OR^{1a}, —C(O)NR^{1b}R^{c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{a}C(O)R^{1d}, —NR^{1a}C(O)OR^{1d}, —NR^{a}C(O)NR^{1b}R^{1c}, —NR^{1a}C(NR^{1d})NR^{1b}R^{1c}, —NR^{a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
each R^{3a} is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
m is an integer of 0, 1, 2, 3, or 4; and
p is an integer of 0, 1, 2, 3, or 4.

69. The compound of claim 68, wherein the compound is a compound of Formula (XXXVI): 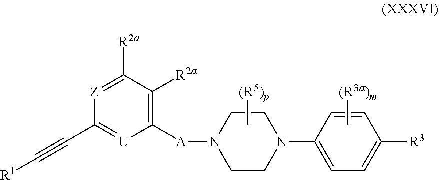
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

70. The compound of any one of claims 1 to 69, wherein A is —C(O)—, —C(O)NR^{1a}—, or —NR^{a}C(O)NR^{1d}—.

71. The compound of any one of claims 1 to 70, wherein A is —C(O)—.

72. The compound of any one of claims 1 to 70, wherein A is —C(O)NR^{1a}—.

73. The compound of claim 72, wherein A is —C(O)NH— or —C(O)N(C₁₋₆ alkyl)-; and wherein the alkyl is optionally substituted with one, two, or three substituents Q.

74. The compound of claim 72 or 73, wherein A is —C(O)NH— or —C(O)N(CH₃)—.

75. The compound of any one of claims 1 to 70, wherein A is —NR^{1a}C(O)NR^{1d}—.

76. The compound of claim 75, wherein A is —NHC(O)NNH—.

77. The compound of claim 63, wherein the compound is a compound of Formula 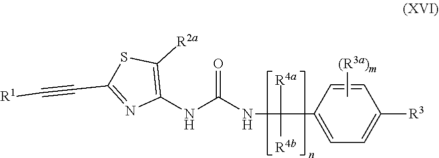
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

78. The compound of claim 65, wherein the compound is a compound of Formula (XXIV): 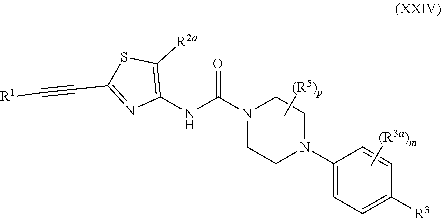
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

79. The compound of any one of claims 62, 63, 66, 67, and 70 to 77, wherein each R^{4a} and R^{4b} is independently hydrogen, C₁₋₆ alkyl, or —NR^{1b}R^{1c}.

80. The compound of claim 79, wherein each R^{4a} and R^{4b} is independently hydrogen, methyl, or amino.

81. The compound of any one of claims 62, 63, 66, 67, 70 to 77, 79, and 80, wherein R^{4a} and R^{4b} together with the carbon atom to which they are attached form C₃₋₁₀ cycloalkylene, optionally substituted with one, two, or three substituents Q.

82. The compound of claim 81, wherein R^{4a} and R^{4b} together with the carbon atom to which they are attached form cyclopropanediyl, optionally substituted with one, two, or three substituents Q.

83. The compound of any one of claims 62, 63, 66, 67, 70 to 77, and 79 to 82, wherein n is an integer of 1, 2, or 3.

84. The compound of claim 1, wherein the compound is a compound of Formula 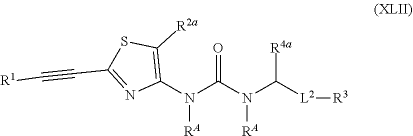
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

85. The compound of claim 1 or 84, wherein the compound is a compound of Formula (XLIII): 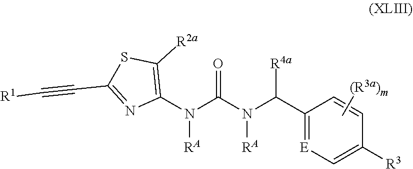
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof; wherein each R^{3a} is independently (i) deuterium, cyano, halo, or nitro; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

86. The compound of claim 85, wherein the compound is a compound of Formula (XLIV): 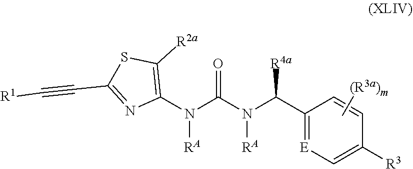
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

87. The compound of claim 85, wherein the compound is a compound of Formula (XLV): 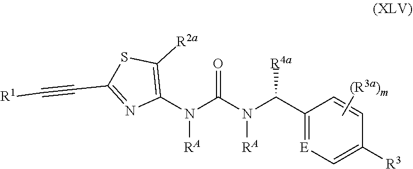
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

88. The compound of any one of claims 84 to 87, wherein R^{A} is hydrogen.

89. The compound of any one of claims 84 to 88, wherein R^{A} is C₁₋₆ alkyl, optionally substituted with one or more substituents Q.

90. The compound of claim 89, wherein R^{A} is methyl.

91. The compound of any one of claims 85 to 90, wherein E is —C(H)═.

92. The compound of any one of claims 85 to 90, wherein E is —N═.

93. The compound of any one of claims 62 to 83 and 85 to 92, wherein each R^{3a} is independently cyano or halo.

94. The compound of claim 93, wherein each R^{3a} is independently cyano, fluoro, or chloro.

95. The compound of any one of claims 62 to 83 and 85 to 94, wherein m is an integer of 0, 1, or 2.

96. The compound of any one of claims 1 to 95, wherein R¹ is hydrogen, deuterium, or C₁₋₆ alkyl, optionally substituted with one or more substituents Q.

97. The compound of any one of claims 1 to 96, wherein R¹ is hydrogen.

98. The compound of any one of claims 1 to 96, wherein R¹ is C₁₋₆ alkyl, optionally substituted with one or more substituents Q.

99. The compound of claim 98, wherein R¹ is methyl or dimethylaminomethyl.

100. The compound of any one of claims 1 to 99, wherein R³ is (i) hydrogen, deuterium, cyano, halo, or nitro; (ii) C₁₋₆ alkyl, C₆₋₁₄ aryl, heteroaryl, or heterocyclyl, each optionally substituted with one or more substituents Q; or (iii) —C(O)OR^{1a}, —OR^{1a}, —NR^{1b}R^{1c}, —NR^{1a}C(O)R^{1d}, —NR^{1a}S(O)₂R^{1d}, —S(O)₂R^{1a}, or —S(O)₂NR^{1b}R^{1c}.

101. The compound of any one of claims 1 to 100, wherein R³ is hydrogen, deuterium, cyano, halo, or nitro.

102. The compound of claim 101, wherein R³ is hydrogen, deuterium, cyano, chloro, bromo, or nitro.

103. The compound of any one of claims 1 to 100, wherein R³ is C₁₋₆ alkyl, monocyclic or bicyclic C₆₋₁₄ aryl, monocyclic, bicyclic, or tricyclic heteroaryl, or bicyclic heterocyclyl; each of which is optionally substituted with one, two, or three substituents Q.

104. The compound of any one of claims 1 to 100 and 103, wherein R³ is C₁₋₆ alkyl, optionally substituted with one, two, or three substituents Q.

105. The compound of claim 104, wherein R³ is methyl or ethyl.

106. The compound of any one of claims 1 to 100 and 103, wherein R³ is monocyclic or bicyclic C₆₋₁₄ aryl, each optionally substituted with one, two, or three substituents Q.

107. The compound of claim 106, wherein R³ is phenyl or 2,3-dihydroindenyl, each of which is optionally substituted with one, two, or three substituents, where each substituent is independently cyano, fluoro, oxo, methyl, cyclopropyl, 1-cyanocyclopropyl, 1-hydroxy-cyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

108. The compound of claim 106 or 107, wherein R³ is phenyl or 2,3-dihydroinden-4-yl, each of which is optionally substituted with one, two, or three substituents, where each substituent is independently cyano, fluoro, oxo, methyl, cyclopropyl, 1-cyanocyclopropyl, 1-hydroxycyclopentyl, cyclopent-1-en-1-yl, azetidin-1-yl, 3-hydroxyazetidin-1-yl, pyrrolidin-1-yl, 3-hydroxypyrrolidin-1-yl, 2-oxopyrrolidin-1-yl, 2-oxoimidazolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

109. The compound of any one of claims 1 to 100 and 103, wherein R³ is monocyclic, bicyclic, or tricyclic heteroaryl, each optionally substituted with one, two, or three substituents Q.

110. The compound of claim 109, wherein R³ is pyrazolyl, thiazolyl, pyridinyl, benzo[b]thiophenyl, benzo[d][1,2,3]thiadiazolyl, benzo[d]thiazolyl, imidazo[1,2-a]pyridinyl, imidazo[1,5-a]pyridinyl, indolyl, indazolyl, thiazolo[4,5-c]pyridinyl, [1,2,3]triazolo[1,5-a]-pyridinyl, [1,2,4]triazolo[1,5-a]pyridinyl, [1,2,4]triazolo[4,3-a]pyridinyl, isoquinolinyl, quinolinyl, quinazolinyl, quinoxalinyl, or 7,8-dihydro-6H-thiazolo[5,4-e]isoindolyl, each of which is optionally substituted with one, two, or three substituents, where each substituent is independently cyano, fluoro, oxo, methyl, 3-hydroxyazetidin-1-yl, pyrrolidin-1-yl, 3-hydroxy-pyrrolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

111. The compound of claim 109 or 110, wherein R³ is pyrazol-3-yl, pyrazol-4-yl, thiazol-4-yl, thiazol-5-yl, pyridin-3-yl, benzo[b]thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, indazol-4-yl, thiazolo[4,5-c]pyridin-7-yl, triazolo[1,5-a]pyridin-4-yl, [1,2,4]triazolo[1,5-a]pyridin-5-yl, [1,2,4]triazolo[1,5-a]-pyridin-8-yl, [1,2,4]triazolo[4,3-a]pyridin-5-yl, [1,2,4]triazolo[4,3-a]pyridin-8-yl, isoquinolin-5-yl, isoquinolin-8-yl, quinolin-5-yl, quinazolin-5-yl, or quinoxalin-5-yl, or 7,8-dihydro-6H-thiazolo[5,4-e]isoindol-5-yl, each of which is optionally substituted with one, two, or three substituents, where each substituent is independently cyano, fluoro, oxo, methyl, 3-hydroxy-azetidin-1-yl, pyrrolidin-1-yl, 3-hydroxypyrrolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

112. The compound of any one of claims 1 to 100 and 103, wherein R³ is bicyclic heterocyclyl, optionally substituted with one, two, or three substituents Q.

113. The compound of claim 112, wherein R³ is 2,3-dihydrobenzo[b]-thiophenyl, isoindolinyl, indolinyl, 2,3-dihydroindazolyl, dihydrobenzo[b]thiophenyl, or 3,4-dihydroquinazolinyl, each of which is optionally substituted with one, two, or three substituents, where each substituent is independently cyano, fluoro, oxo, methyl, 3-hydroxyazetidin-1-yl, pyrrolidin-1-yl, 3-hydroxypyrrolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

114. The compound of claim 112 or 113, wherein R³ is 2,3-dihydrobenzo[b]-thiophen-7-yl, isoindolin-4-yl, indolin-4-yl, 2,3-dihydroindazol-4-yl, dihydrobenzo[b]thiophen-7-yl, or 3,4-dihydroquinazolin-5-yl, each of which is optionally substituted with one, two, or three substituents, where each substituent is independently cyano, fluoro, oxo, methyl, 3-hydroxyazetidin-1-yl, pyrrolidin-1-yl, 3-hydroxypyrrolidin-1-yl, 2-oxooxazolidin-3-yl, methoxycarbonyl, carbamoyl, methylcarbamoyl, hydroxyl, (3-hydroxycyclobutyl)amino, oxetan-3-ylamino, methylsulfonyl, methylsulfamoyl, or dimethylsulfamoyl.

115. The compound of any one of claims 1 to 100 and 103, wherein R³ is (i) hydrogen, deuterium, cyano, chloro, bromo, or nitro; (ii) methyl, methoxycarbonylmethyl, carbamoyl-methyl, hydroxymethyl, 2-methoxycarbonylethyl, 2-hydroxylethyl, phenyl, 2-cyanophenyl, 3-cyclopropylphenyl, 3-(1-cyanocyclopropyl)phenyl, 3-(1-hydroxycyclopentyl)phenyl, 3-(cyclopent-1-en-1-yl)phenyl, 3-(azetidin-1-yl)phenyl, 3-(pyrrolidin-1-yl)phenyl, 3-(3-hydroxypyrrolidin-1-yl)phenyl, 3-(2-oxopyrrolidin-1-yl)phenyl, 3-(2-oxoimidazolidin-1-yl)phenyl, 3-(2-oxooxazolidin-3-yl)phenyl, 3-(3-hydroxycyclobutyl)-aminophenyl, 3-(oxetan-3-ylamino)phenyl, 3-(3-hydroxyazetidin-1-yl)phenyl, 3-carbamoylphenyl, 2-methylcarbamoyl-phenyl, 3-methylcarbamoylphenyl, 2-methylsulfamoylphenyl, 2-dimethylsulfamoylphenyl, 2-methyl-sulfonylphenyl, 3-oxo-2,3-dihydro-1H-inden-4-yl, pyrazol-3-yl, pyrazol-4-yl, 1-methylpyrazol-3-yl, 1-methylpyrazol-4-yl, thiazol-4-yl, thiazol-5-yl, pyridin-3-yl, 1,1-dioxidobenzo[b]-thiophen-7-yl, benzo[d][1,2,3]thiadiazol-7-yl, benzo[d]thiazol-4-yl, benzo[d]thiazol-5-yl, benzo[d]thiazol-6-yl, benzo[d]thiazol-7-yl, 6-fluorobenzo[d]thiazol-5-yl, 6-cyanobenzo[d]thiazol-7-yl, 6-fluorobenzo[d]thiazol-7-yl, 5-methoxycarbonylbenzo[d]-thiazol-7-yl, 6-methoxycarbonylbenzo[d]thiazol-7-yl, 5-carbamoylbenzo[d]thiazol-7-yl, 6-carbamoylbenzo[d]thiazol-7-yl, 5-methylcarbamoylbenzo[d]thiazol-7-yl, 6-methylcarbamoyl-benzo[d]thiazol-7-yl, 2-aminobenzo[d]thiazol-7-yl, 2-amino-6-cyanobenzo[d]thiazol-7-yl, imidazo[1,2-a]pyridin-5-yl, imidazo[1,2-a]pyridin-8-yl, imidazo[1,5-a]pyridin-5-yl, imidazo[1,5-a]pyridin-8-yl, indol-4-yl, 1-methylindol-4-yl, indazol-4-yl, 1-methylindazol-4-yl, 2-methylindazol-4-yl, 1,5-dimethyl-indazol-4-yl, 1-methyl-6-methoxycarbonylindazol-4-yl, 1-methyl-6-carbamoylindazol-4-yl, 1-methyl-6-methylcarbamoylindazol-4-yl, 1-methyl-6-dimethylcarbamoylindazol-4-yl, thiazolo[4,5-c]pyridin-7-yl, [1,2,3]triazolo[1,5-a]pyridin-4-yl, triazolo[1,5-a]pyridin-5-yl, 2-amino-[1,2,4]triazolo[1,5-a]pyridin-5-yl, [1,2,4]triazolo[1,5-a]pyridin-8-yl, [1,2,4]triazolo[4,3-a]pyridin-5-yl, [1,2,4]triazolo[4,3-a]pyridin-8-yl, isoquinolin-5-yl, 1-hydroxyisoquinolin-8-yl, quinolin-5-yl, quinazolin-5-yl, 4-hydroxy-quinazolin-5-yl, quinoxalin-5-yl, 8-oxo-7,8-dihydro-6H-thiazolo[5,4-e]isoindol-5-yl, 3-hydroxy-1,1-dioxido-2,3-dihydrobenzo[b]thiophen-7-yl, 1,1-dioxido-3-oxo-2,3-dihydrobenzo[b]-thiophen-7-yl, 1-oxoisoindolin-4-yl, 3-oxoisoindolin-4-yl, 2-methyl-1-oxoisoindolin-4-yl, 2,3-dioxoindolin-4-yl, 2-oxoindolin-4-yl, 1-methyl-2-oxoindolin-4-yl, 1-methyl-3-oxo-2,3-dihydro-1H-indazol-4-yl, 2,2-difluoro-1,1-dioxido-3-oxo-2,3-dihydrobenzo[b]-thiophen-7-yl, or 3-methyl-4-oxo-3,4-dihydroquinazolin-5-yl; or (iii) methoxycarbonyl, hydroxyl, methoxy, amino, acetamido, methylsulfonamido, methylsulfonyl, or methylsulfamoyl.

116. A compound of;
1-(4-(1,1-dioxidobenzo[b]thiophen-7-yl)benzyl)-3-(2-ethynylthiazol-4-yl)urea A1;
1-(2-ethynylthiazol-4-yl)-3-(4-(3-hydroxy-1,1-dioxido-2,3-dihydrobenzo[b]-thiophen-7-yl)benzyl)urea A2;
1-(4-(1,1-dioxido-3-oxo-2,3-dihydrobenzo[b]thiophen-7-yl)benzyl)-3-(2-ethynylthiazol-4-yl)urea A3;
1-(2-ethynylthiazol-4-yl)-3-(4-(8-oxo-7,8-dihydro-6H-thiazolo[5,4-e]isoindol-5-yl)-benzyl)urea A4;
(S)-1-(2-ethynylthiazol-4-yl)-3-((3′-(3-hydroxypyrrolidin-1-yl)-[1,1′-biphenyl]-4-yl)-methyl)urea A5;
1-(4-(benzo[d]thiazol-7-yl)benzyl)-3-(2-ethynylthiazol-4-yl)urea A6;
1-(2-ethynylthiazol-4-yl)-3-((3′-(pyrrolidin-1-yl)-[1,1′-biphenyl]-4-yl)methyl)urea A7;
1-([1,1′-biphenyl]-4-ylmethyl)-3-(2-ethynylthiazol-4-yl)urea A8;
1-(2-ethynylthiazol-4-yl)-3-(4-(4-hydroxyquinazolin-5-yl)benzyl)urea A9;
4′-((3-(2-ethynylthiazol-4-yl)ureido)methyl)-[1,1′-biphenyl]-3-carboxamide A10;
1-(2-ethynylthiazol-4-yl)-3-(4-(3-methyl-4-oxo-3,4-dihydroquinazolin-5-yl)benzyl)urea A11;
1-(2-ethynylthiazol-4-yl)-3-(4-(6-fluorobenzo[d]thiazol-5-yl)benzyl)urea A12;
4′-((3-(2-ethynylthiazol-4-yl)ureido)methyl)-N-methyl-[1,1′-biphenyl]-2-sulfonamide A13;
4′-((3-(2-ethynylthiazol-4-yl)ureido)methyl)-N-methyl-[1,1′-biphenyl]-3-carboxamide A14;
1-(4-(1,5-dimethyl-1H-indazol-4-yl)benzyl)-3-(2-ethynylthiazol-4-yl)urea A15;
1-(2-ethynylthiazol-4-yl)-3-(4-(1-methyl-1H-indazol-4-yl)benzyl)urea A16;
7-(4-((3-(2-ethynylthiazol-4-yl)ureido)methyl)phenyl)-N-methylbenzo[d]thiazole-6-carboxamide A17;
1-(2-ethynylthiazol-4-yl)-3-(4-(1-hydroxyisoquinolin-8-yl)benzyl)urea A18;
7-(4-((3-(2-ethynylthiazol-4-yl)ureido)methyl)phenyl)benzo[d]thiazole-6-carboxamide A19;
4-(4-((3-(2-ethynylthiazol-4-yl)ureido)methyl)phenyl)-1-methyl-1H-indazole-6-carboxamide A20;
4-(4-((3-(2-ethynylthiazol-4-yl)ureido)methyl)phenyl)-N,1-dimethyl-1H-indazole-6-carboxamide A21;
methyl 4-(4-((3-(2-ethynylthiazol-4-yl)ureido)methyl)phenyl)-1-methyl-1H-indazole-6-carboxylate A22; or
4-(4-((3-(2-ethynylthiazol-4-yl)ureido)methyl)phenyl)-N,N,1-trimethyl-1H-indazole-6-carboxamide A23;
1-(2-ethynylthiazol-4-yl)-3-(4-(1-methyl-3-oxo-2,3-dihydro-1H-indazol-4-yl)benzyl)urea A24;
(S)-1-(1-(4-(benzo[d]thiazol-7-yl)phenyl)ethyl)-3-(2-ethynylthiazol-4-yl)urea A25;
(R)-1-(1-(4-(benzo[d]thiazol-7-yl)phenyl)ethyl)-3-(2-ethynylthiazol-4-yl)urea A26;
1-(4-(2,2-difluoro-1,1-dioxido-3-oxo-2,3-dihydrobenzo[b]thiophen-7-yl)benzyl)-3-(2-ethynylthiazol-4-yl)urea A27;
1-(2-ethynylthiazol-4-yl)-3-((3′-(oxetan-3-ylamino)-[1,1′-biphenyl]-4-yl)methyl)urea A28;
1-((2′-cyano-[1,1′-biphenyl]-4-yl)methyl)-3-(2-ethynylthiazol-4-yl)urea A29;
1-(2-ethynylthiazol-4-yl)-3-(4-(pyrrolidin-1-yl)benzyl)urea A30;
1-(4-((3-(2-ethynylthiazol-4-yl)ureido)methyl)phenyl)pyrrolidine-2-carboxamide A31;
(S)-1-(4-(2-cyanopyrrolidin-1-yl)benzyl)-3-(2-ethynylthiazol-4-yl)urea A32;
(R)-1-(4-(2-cyanopyrrolidin-1-yl)benzyl)-3-(2-ethynylthiazol-4-yl)urea A33;
1-(2-(3-(2-cyanophenyl)azetidin-1-yl)-2-oxoethyl)-3-(2-ethynylthiazol-4-yl)urea A34;
1-(2-(4-(2-cyanophenyl)piperidin-1-yl)-2-oxoethyl)-3-(2-ethynylthiazol-4-yl)urea A35;
1-(2-(4-(2-cyanophenyl)piperazin-1-yl)-2-oxoethyl)-3-(2-ethynylthiazol-4-yl)urea A36;
1-((1-(2-cyanophenyl)piperidin-4-yl)methyl)-3-(2-ethynylthiazol-4-yl)urea A37;
1-(adamantan-1-ylmethyl)-3-(2-ethynylthiazol-4-yl)urea A38;
1-(1-(4-(benzo[d]thiazol-7-yl)phenyl)piperidin-4-yl)-3-(2-ethynylthiazol-4-yl)urea A39;
(R)-1-(1-(4-(benzo[d]thiazol-7-yl)phenyl)-2-hydroxyethyl)-3-(2-ethynylthiazol-4-yl)urea A40;
(S)-1-(1-(4-(benzo[d]thiazol-7-yl)phenyl)-2-hydroxyethyl)-3-(2-ethynylthiazol-4-yl)urea A41;
(R)-1-(2-ethynylthiazol-4-yl)-3-(2-hydroxy-1-(3′-(pyrrolidin-1-yl)-[1,1′-biphenyl]-4-yl)-ethyl)urea A42;
(R)-2-(4-(2-(1-cyanocyclopropyl)pyridin-3-yl)phenyl)-2-(3-(2-ethynylthiazol-4-yl)-ureido)-N-methylacetamide A43;
(R)-1-(1-(2′-cyano-[1,1′-biphenyl]-4-yl)-2-hydroxyethyl)-3-(2-ethynylthiazol-4-yl)urea A44;
(R)-1-(1-(5-(2-cyanophenyl)pyridin-2-yl)-2-hydroxyethyl)-3-(2-ethynylthiazol-4-yl)urea A45;
(R)-2-(2′-cyano-[1,1′-biphenyl]-4-yl)-2-(3-(2-ethynylthiazol-4-yl)ureido)ethyl carbamate A46;
(R)-2-(4-(3-cyanopyridin-2-yl)phenyl)-2-(3-(2-ethynylthiazol-4-yl)ureido)ethyl carbamate A47;
(R)-2-(4-(4-cyanopyridin-2-yl)phenyl)-2-(3-(2-ethynylthiazol-4-yl)ureido)ethyl carbamate A48;
(R)-2-(3-(2-ethynylthiazol-4-yl)ureido)-2-(4-(isoquinolin-8-yl)phenyl)ethyl carbamate A49;
(R)-2-(3-(2-ethynylthiazol-4-yl)ureido)-2-(4-(quinazolin-8-yl)phenyl)ethyl carbamate A50;
(R)-2-(3-(2-ethynylthiazol-4-yl)ureido)-2-(4-(quinoxalin-5-yl)phenyl)ethyl carbamate A51;
(R)-1-(1-(4-(7-cyanoquinolin-8-yl)phenyl)-2-hydroxyethyl)-3-(2-ethynylthiazol-4-yl)urea A52;
(R)-1-(1-(4-(3-cyano-1-methyl-1H-indazol-4-yl)phenyl)-2-hydroxyethyl)-3-(2-ethynyl-thiazol-4-yl)urea A53;
(R)-1-(1-(2′-(1-cyanocyclopropyl)-[1,1′-biphenyl]-4-yl)-2-hydroxyethyl)-3-(2-ethynyl-thiazol-4-yl)urea A54;
(R)-2-(2′-(1-cyanocyclopropyl)-[1,1′-biphenyl]-4-yl)-2-(3-(2-ethynylthiazol-4-yl)ureido)-ethyl carbamate A55;
(R)-2-(4-(6-(1-cyanocyclopropyl)pyridin-2-yl)phenyl)-2-(3-(2-ethynylthiazol-4-yl)-ureido)ethyl carbamate A56;
(R)-2-(4-(4-(1-cyanocyclopropyl)pyridin-2-yl)phenyl)-2-(3-(2-ethynylthiazol-4-yl)-ureido)ethyl carbamate A57;
(R)-2-(4-(3-(1-cyanocyclopropyl)pyridin-2-yl)phenyl)-2-(3-(2-ethynylthiazol-4-yl)-ureido)ethyl carbamate A58;
(R)-1-(1-(6-(2-(1-cyanocyclopropyl)phenyl)pyridin-3-yl)-2-hydroxyethyl)-3-(2-ethynyl-thiazol-4-yl)urea A59;
(R)-2-(5-(2-(1-cyanocyclopropyl)phenyl)pyridin-2-yl)-2-(3-(2-ethynylthiazol-4-yl)-ureido)ethyl carbamate A60;
(S)-2-(5-(2-(1-cyanocyclopropyl)phenyl)pyridin-2-yl)-2-(3-(2-ethynylthiazol-4-yl)-ureido)ethyl carbamate A61;
(R)-1-(1-(4-(2-(1-cyanocyclopropyl)pyridin-3-yl)phenyl)-2-hydroxyethyl)-3-(2-ethynyl-thiazol-4-yl)urea A62;
(R)-2-(4-(2-(1-cyanocyclopropyl)pyridin-3-yl)phenyl)-2-(3-(2-ethynylthiazol-4-yl)-ureido)ethyl carbamate A63;
(R)-1-(1-(4-(2-(1-cyanocyclopropyl)-5-fluoropyridin-3-yl)phenyl)-2-hydroxy-ethyl)-3-(2-ethynylthiazol-4-yl)urea A64;
(R)-1-(1-(3-chloro-4-(2-(1-cyanocyclopropyl)pyridin-3-yl)phenyl)-2-hydroxy-ethyl)-3-(2-ethynylthiazol-4-yl)urea A65;
(S)-1-(1-(3-chloro-4-(2-(1-cyanocyclopropyl)pyridin-3-yl)phenyl)-2-hydroxy-ethyl)-3-(2-ethynylthiazol-4-yl)urea A66;
(R)-1-(1-(3-chloro-4-(2-(1-cyanocyclopropyl)pyridin-3-yl)phenyl)-2-hydroxy-ethyl)-3-(2-ethynylthiazol-4-yl)-1-methylurea A67;
(R)-1-(1-(4-(2-(1-cyanocyclopropyl)pyridin-3-yl)-3-fluorophenyl)-2-hydroxy-ethyl)-3-(2-ethynylthiazol-4-yl)urea A68;
(R)-2-(3-chloro-4-(2-(1-cyanocyclopropyl)pyridin-3-yl)phenyl)-2-(3-(2-ethynyl-thiazol-4-yl)ureido)ethyl carbamate A69;
(R)-2-(3-chloro-4-(2-(1-cyanocyclopropyl)pyridin-3-yl)phenyl)-2-(3-(2-ethynyl-thiazol-4-yl)-1-methylureido)ethyl carbamate A70;
(R)-1-(1-(3-chloro-4-(2-(1-cyanocyclopropyl)pyridin-3-yl)phenyl)-2-hydroxy-ethyl)-3-(5-ethynyl-1,3,4-thiadiazol-2-yl)urea A71;
(R)-1-(1-(3-chloro-4-(2-(1-cyanocyclopropyl)pyridin-3-yl)phenyl)-2-(methyl-sulfonyl)-ethyl)-3-(2-ethynylthiazol-4-yl)urea A72;
(R)-2-(3-chloro-4-(2-(1-cyanocyclopropyl)pyridin-3-yl)phenyl)-2-(3-(2-ethynyl-thiazol-4-yl)ureido)ethanesulfonamide A73;
(R)-2-(3-chloro-4-(2-(1-cyanocyclopropyl)pyridin-3-yl)phenyl)-2-(3-(2-ethynyl-thiazol-4-yl)ureido)-N-methylacetamide A74;
(R)-1-(1-(5-(2-(1-cyanocyclopropyl)-4-fluorophenyl)pyridin-2-yl)-2-hydroxy-ethyl)-3-(2-ethynylthiazol-4-yl)urea A75;
(R)-2-(5-(2-(1-cyanocyclopropyl)-4-fluorophenyl)pyridin-2-yl)-2-(3-(2-ethynyl-thiazol-4-yl)ureido)ethyl carbamate A76;
(R)-1-(1-(5-(2-(1-cyanocyclopropyl)-5-fluorophenyl)pyridin-2-yl)-2-hydroxyethyl)-3-(2-ethynylthiazol-4-yl)urea A77;
(R)-1-(1-(5-(2-(1-cyanocyclopropyl)-6-fluorophenyl)pyridin-2-yl)-2-hydroxy-ethyl)-3-(2-ethynylthiazol-4-yl)urea A78;
(R)-1-(1-(5-(2-(1-cyanocyclopropyl)-4,6-difluorophenyl)pyridin-2-yl)-2-hydroxyethyl)-3-(2-ethynylthiazol-4-yl)urea A79;
1-((1R)-1-(5-(2-(2,2-difluorocyclopropyl)phenyl)pyridin-2-yl)-2-hydroxyethyl)-3-(2-ethynylthiazol-4-yl)urea A80;
1-((1R)-1-(5-(2-(2,2-difluorocyclopropyl)-4-fluorophenyl)pyridin-2-yl)-2-hydroxyethyl)-3-(2-ethynylthiazol-4-yl)urea A81;
1-((1R)-1-(5-(2-(2,2-difluorocyclopropyl)-4-fluorophenyl)pyridin-2-yl)-2-hydroxyethyl)-3-(2-ethynylthiazol-4-yl)-1-methylurea A82;
(2R)-2-(5-(2-(2,2-difluorocyclopropyl)-4-fluorophenyl)pyridin-2-yl)-2-(3-(2-ethynyl-thiazol-4-yl)ureido)ethyl carbamate A83;
(2R)-2-(5-(2-(2,2-difluorocyclopropyl)-4-fluorophenyl)pyridin-2-yl)-2-(3-(2-ethynyl-thiazol-4-yl)-1-methylureido)ethyl carbamate A84;
1-((1R)-1-(5-(2-(2,2-difluorocyclopropyl)-4-fluorophenyl)pyridin-2-yl)-2-hydroxyethyl)-3-(5-ethynyl-1,3,4-thiadiazol-2-yl)urea A85;
1-((1R)-1-(5-(2-(2,2-difluorocyclopropyl)-4-fluorophenyl)pyridin-2-yl)-2-hydroxyethyl)-3-(5-ethynyl-1,3,4-thiadiazol-2-yl)-1-methylurea A86;
(2R)-2-(5-(2-(2,2-difluorocyclopropyl)-4-fluorophenyl)pyridin-2-yl)-2-(3-(5-ethynyl-1,3,4-thiadiazol-2-yl)ureido)ethyl carbamate A87;
(R)-1-(2-ethynylthiazol-4-yl)-3-(2-hydroxy-1-(4-(6-(pyrrolidin-1-yl)pyridin-2-yl)phenyl)-ethyl)urea A88;
(R)-1-(2-ethynylthiazol-4-yl)-3-(2-hydroxy-1-(6-(pyrrolidin-1-yl)-[2,3′-bipyridin]-6′-yl)-ethyl)urea A89;
(R)-2-(3-(2-ethynylthiazol-5-yl)ureido)-2-(6-(pyrrolidin-1-yl)-[2,3′-bipyridin]-6′-yl)ethyl carbamate A90;
(R)-3-(2-ethynylthiazol-4-yl)-1-(2-hydroxy-1-(6-(pyrrolidin-1-yl)-[2,3′-bipyridin]-6′-yl)-ethyl)-1-methylurea A91;
(R)-2-(3-(2-ethynylthiazol-4-yl)-1-methylureido)-2-(6-(pyrrolidin-1-yl)-[2,3′-bipyridin]-6′-yl)ethyl carbamate A92;
(R)-1-(5-ethynyl-1,3,4-thiadiazol-2-yl)-3-(2-hydroxy-1-(4-(6-(pyrrolidin-1-yl)-pyridin-2-yl)phenyl)ethyl)urea A93;
(R)-2-(3-(5-ethynyl-1,3,4-thiadiazol-2-yl)ureido)-2-(4-(6-(pyrrolidin-1-yl)-pyridin-2-yl)-phenyl)ethyl carbamate A94;
(R)-2-(3-(5-ethynyl-1,3,4-thiadiazol-2-yl)-1-methylureido)-2-(4-(6-(pyrrolidin-1-yl)-pyridin-2-yl)phenyl)ethyl carbamate A95;
(R)-1-(5-ethynyl-1,3,4-thiadiazol-2-yl)-3-(2-hydroxy-1-(6-(pyrrolidin-1-yl)-[2,3′-bipyridin]-6′-yl)ethyl)urea A96;
(R)-2-(3-(5-ethynyl-1,3,4-thiadiazol-2-yl)ureido)-2-(6-(pyrrolidin-1-yl)-[2,3′-bipyridin]-6′-yl)ethyl carbamate A97;
(R)-1-(1-(4-(2-(dimethylamino)pyridin-3-yl)phenyl)-2-hydroxyethyl)-3-(2-ethynyl-thiazol-4-yl)urea A98;
(R)-2-(4-(2-(dimethylamino)pyridin-3-yl)phenyl)-2-(3-(2-ethynylthiazol-4-yl)-ureido)-ethyl carbamate A99;
(R)-1-(1-(3-chloro-4-(2-(dimethylamino)pyridin-3-yl)phenyl)-2-hydroxyethyl)-3-(2-ethynylthiazol-4-yl)urea A100;
(R)-2-(3-chloro-4-(2-(dimethylamino)pyridin-3-yl)phenyl)-2-(3-(2-ethynyl-thiazol-4-yl)-ureido)ethyl carbamate A101;
(R)-1-(1-(4-(2-(3,3-difluoroazetidin-1-yl)pyridin-3-yl)phenyl)-2-hydroxyethyl)-3-(2-ethynylthiazol-4-yl)urea A102;
(R)-2-(4-(2-(3,3-difluoroazetidin-1-yl)pyridin-3-yl)phenyl)-2-(3-(2-ethynyl-thiazol-4-yl)-ureido)ethyl carbamate A103;
(R)-1-(2-ethynylthiazol-4-yl)-3-(2-hydroxy-1-(4-(1-hydroxyisoquinolin-8-yl)-phenyl)-ethyl)urea A104;
(R)-2-(3-(2-ethynylthiazol-4-yl)ureido)-2-(4-(1-hydroxyisoquinolin-8-yl)phenyl)-ethyl carbamate A105;
(R)-1-(2-ethynylthiazol-4-yl)-3-(2-hydroxy-1-(5-(1-hydroxyisoquinolin-8-yl)-pyridin-2-yl)ethyl)urea A106;
(R)-1-(1-(3-chloro-4-(1-hydroxyisoquinolin-8-yl)phenyl)-2-hydroxyethyl)-3-(2-ethynyl-thiazol-4-yl)urea A107;
(R)-1-(5-ethynyl-1,3,4-thiadiazol-2-yl)-3-(2-hydroxy-1-(4-(1-hydroxyisoquinolin-8-yl)-phenyl)ethyl)urea A108;
(R)-1-(2-ethynylthiazol-4-yl)-3-(2-hydroxy-1-(4-(4-hydroxyquinazolin-5-yl)phenyl)-ethyl)urea A109;
(R)-1-(1-(3-chloro-4-(4-hydroxyquinazolin-5-yl)phenyl)-2-hydroxyethyl)-3-(2-ethynyl-thiazol-4-yl)urea A110;
(R)-1-(1-(4-(benzo[d]thiazol-7-yl)phenyl)-2-hydroxyethyl)-3-(2-ethynylthiazol-4-yl)-1-methylurea A111;
(R)-3-(1-(4-(benzo[d]thiazol-7-yl)phenyl)-2-hydroxyethyl)-1-(2-ethynylthiazol-4-yl)-1-methylurea A112;
(R)-1-(1-(4-(benzo[d]thiazol-7-yl)phenyl)-2-hydroxyethyl)-3-(2-ethynylthiazol-4-yl)-1,3-dimethylurea A113;
(R)-1-(1-(5-(benzo[d]thiazol-7-yl)pyridin-2-yl)-2-hydroxyethyl)-3-(2-ethynyl-thiazol-4-yl)urea A114;
(R)-2-(3-chloro-4-(4-fluorobenzo[d]thiazol-7-yl)phenyl)-2-(3-(2-ethynylthiazol-4-yl)-ureido)ethane-1-sulfonamide A115;
(R)-2-(4-(benzo[d]thiazol-7-yl)phenyl)-2-(3-(2-ethynylthiazol-4-yl)ureido)ethyl L-valinate A116;
(5Z,8Z,11Z,14Z)—(R)-2-(4-(benzo[d]thiazol-7-yl)phenyl)-2-(3-(2-ethynylthiazol-4-yl)-ureido)ethyl icosa-5,8,11,14-tetraenoate A117;
(R)-1-(1-(4-(Benzo[d]thiazol-7-yl)phenyl)-2-hydroxyethyl)-3-(2-ethynylthiazol-5-yl)urea A118;
(R)-1-(1-(4-(Benzo[d]thiazol-7-yl)phenyl)-2-hydroxyethyl)-3-(2-ethynyl-5-methyl-thiazol-4-yl)urea A119;
(R)-1-(1-(4-(Benzo[d]thiazol-7-yl)phenyl)-2-hydroxyethyl)-3-(5-ethynyl-1,3,4-thiadiazol-2-yl)urea A120;
(R)-1-(1-(4-(Benzo[d]thiazol-7-yl)phenyl)-2-hydroxyethyl)-3-(3-ethynyl-1,2,4-thiadiazol-5-yl)urea A121;
(R)-1-(1-(4-(Benzo[d]thiazol-7-yl)phenyl)-2-hydroxyethyl)-3-(5-ethynyl-1,2,4-thiadiazol-3-yl)urea A122;
(R)-1-(4-ethynylpyrimidin-2-yl)-3-(2-hydroxy-1-(4-(6-(pyrrolidin-1-yl)pyridin-2-yl)-phenyl)ethyl)urea A123;
(R)-1-(6-ethynylpyridin-2-yl)-3-(2-hydroxy-1-(4-(6-(pyrrolidin-1-yl)pyridin-2-yl)-phenyl)ethyl)urea A124;
(R)-1-(2-ethynylpyrimidin-4-yl)-3-(2-hydroxy-1-(4-(6-(pyrrolidin-1-yl)pyridin-2-yl)-phenyl)ethyl)urea A125;
(R)-1-(1-(4-(3,3-difluoroazetidin-1-yl)phenyl)-2-hydroxyethyl)-3-(2-ethynyl-thiazol-4-yl)urea A126;
(R)-2-(4-(3,3-difluoroazetidin-1-yl)phenyl)-2-(3-(2-ethynylthiazol-4-yl)ureido)-ethyl carbamate A127;
(R)-1-(2-ethynylthiazol-4-yl)-3-(2-hydroxy-1-(4-(pyrrolidin-1-yl)phenyl)ethyl)-urea A128;
(R)-1-(2-ethynylthiazol-4-yl)-3-(2-hydroxy-1-(4-(2-oxopyrrolidin-1-yl)phenyl)-ethyl)urea A129;
(R)-1-(1-(4-(3,3-difluoropyrrolidin-1-yl)phenyl)-2-hydroxyethyl)-3-(2-ethynyl-thiazol-4-yl)urea A130;
(R)-1-(2-ethynylthiazol-4-yl)-3-(2-hydroxy-1-(4-(2-oxopiperidin-1-yl)phenyl)-ethyl)urea A131;
(R)-1-(2-ethynylthiazol-4-yl)-3-(2-hydroxy-1-(4-(2-oxopyridin-1(2H)-yl)phenyl)-ethyl)-urea A132;
(R)-1-(2-ethynylthiazol-4-yl)-3-(2-hydroxy-1-(4-morpholinophenyl)ethyl)urea A133;
(R)-1-(2-ethynylthiazol-4-yl)-3-(2-hydroxy-1-(4-(3-oxomorpholino)phenyl)-ethyl)urea A134;
(R)-1-(2-ethynylthiazol-4-yl)-3-(2-hydroxy-1-(5-(piperidin-1-yl)pyridin-2-yl)-ethyl)urea A135;
(R)-1-(1-(5-(3,3-difluoropiperidin-1-yl)pyridin-2-yl)-2-hydroxyethyl)-3-(2-ethynyl-thiazol-4-yl)urea A136;
(R)-1-(1-(4-(azepan-1-yl)phenyl)-2-hydroxyethyl)-3-(2-ethynylthiazol-4-yl)urea A137;
(R)-1-(2-ethynylthiazol-4-yl)-3-(2-hydroxy-1-(4-(2-oxoazepan-1-yl)phenyl)-ethyl)urea A138;
(R)-1-(2-ethynylthiazol-4-yl)-3-(2-hydroxy-1-(4-(1-methyl-1,5,6,7-tetrahydro-4H-pyrazolo[4,3-b]pyridin-4-yl)phenyl)ethyl)urea A139;
(R)-1-(2-ethynylthiazol-4-yl)-3-(2-hydroxy-1-(4-(2-methyl-2,5,6,7-tetrahydro-4H-pyrazolo[4,3-b]pyridin-4-yl)phenyl)ethyl)urea A140;
(R)-1-(1-(6′-cyano-2′,3′,4′,5′-tetrahydro-[1,1′-biphenyl]-4-yl)-2-hydroxyethyl)-3-(2-ethynylthiazol-4-yl)urea A141;
(R)-1-(1-(5-(2-cyanocyclohex-1-en-1-yl)pyridin-2-yl)-2-hydroxyethyl)-3-(2-ethynyl-thiazol-4-yl)urea A142;
1-((1R)-1-(4-(1-acetylpiperidin-2-yl)phenyl)-2-hydroxyethyl)-3-(2-ethynyl-thiazol-4-yl)-urea A143;
(R)-1-(1-(3-(2-cyanophenyl)azetidin-1-yl)-3-hydroxy-1-oxopropan-2-yl)-3-(2-ethynyl-thiazol-4-yl)urea A144;
(S)-1-(1-(3-(2-cyanophenyl)azetidin-1-yl)-3-hydroxy-1-oxopropan-2-yl)-3-(2-ethynyl-thiazol-4-yl)urea A145;
(R)-1-(1-(4-(2-cyanophenyl)piperidin-1-yl)-3-hydroxy-1-oxopropan-2-yl)-3-(2-ethynyl-thiazol-4-yl)urea A146;
(S)-1-(1-(4-(2-cyanophenyl)piperidin-1-yl)-3-hydroxy-1-oxopropan-2-yl)-3-(2-ethynyl-thiazol-4-yl)urea A147;
(R)-1-(1-(4-(2-cyanophenyl)piperazin-1-yl)-3-hydroxy-1-oxopropan-2-yl)-3-(2-ethynyl-thiazol-4-yl)urea A148;
(S)-1-(1-(4-(2-cyanophenyl)piperazin-1-yl)-3-hydroxy-1-oxopropan-2-yl)-3-(2-ethynyl-thiazol-4-yl)urea A149;
(R)-1-(1-(1-(2-cyanophenyl)piperidin-4-yl)-2-hydroxyethyl)-3-(2-ethynylthiazol-4-yl)-urea A150;
(S)-1-(1-(1-(2-cyanophenyl)piperidin-4-yl)-2-hydroxyethyl)-3-(2-ethynylthiazol-4-yl)-urea A151;
(S)-1-(2-ethynylthiazol-4-yl)-3-(3-hydroxy-1-oxo-1-(6-azaspiro[2.5]octan-6-yl)propan-2-yl)urea A152;
(S)-1-(2-ethynylthiazol-4-yl)-3-(3-hydroxy-1-oxo-1-(7-azaspiro[3.5]nonan-7-yl)propan-2-yl)urea A153;
(S)-1-(2-ethynylthiazol-4-yl)-3-(3-hydroxy-1-oxo-1-(8-azaspiro[4.5]decan-8-yl)propan-2-yl)urea A154;
(R)-1-(2-ethynylthiazol-4-yl)-3-(3-hydroxy-1-oxo-1-(8-azaspiro[4.5]decan-8-yl)propan-2-yl)urea A155;
(S)-1-(2-ethynylthiazol-5-yl)-3-(3-hydroxy-1-oxo-1-(1-oxo-8-azaspiro[4.5]decan-8-yl)-propan-2-yl)urea A156;
(S)-1-(2-ethynylthiazol-4-yl)-3-(3-hydroxy-1-oxo-1-(3-azaspiro[5.5]undecan-3-yl)-propan-2-yl)urea A157;
(S)-1-(1-(9,9-difluoro-3-azaspiro[5.5]undecan-3-yl)-3-hydroxy-1-oxopropan-2-yl)-3-(2-ethynylthiazol-4-yl)urea A158;
(S)-1-(1-(4-(3,3-difluoroazetidin-1-yl)piperidin-1-yl)-3-hydroxy-1-oxopropan-2-yl)-3-(2-ethynylthiazol-4-yl)urea A159;
(R)-1-(1-(4-(3,3-difluoroazetidin-1-yl)piperidin-1-yl)-3-hydroxy-1-oxopropan-2-yl)-3-(2-ethynylthiazol-4-yl)urea A160;
(S)-1-(1-(4,4-difluoropiperidin-1-yl)-3-hydroxy-1-oxopropan-2-yl)-3-(2-ethynyl-thiazol-4-yl)urea A161;
1-(6-chloro-8-fluoro-7-(2-fluoro-6-methoxyphenyl)quinazolin-4-yl)-3-(2-ethynylthiazol-4-yl)urea A162;
1-(2-ethynylthiazol-4-yl)-3-(7-(2-fluoro-6-hydroxyphenyl)quinazolin-4-yl)urea A163;
1-(2-ethynylthiazol-4-yl)-3-(7-(2-fluoro-6-methoxyphenyl)quinazolin-4-yl)urea A164;
1-(7-(benzo[d]thiazol-7-yl)-6-chloro-8-fluoroquinazolin-4-yl)-3-(2-ethynyl-thiazol-4-yl)urea A165;
1-(7-(2-(1-cyanocyclopropyl)pyridin-3-yl)quinazolin-4-yl)-3-(2-ethynylthiazol-4-yl)urea A166;
1-((3′-(1-cyanocyclopropyl)-[1,1′-biphenyl]-4-yl)methyl)-3-(2-ethynylthiazol-4-yl)urea A167;
1-(2-ethynylthiazol-4-yl)-3-((3′-(1-hydroxycyclopropyl)-[1,1′-biphenyl]-4-yl)methyl)urea A168;
1-(2-ethynylthiazol-4-yl)-3-((3′-propionyl-[1,1′-biphenyl]-4-yl)methyl)urea A169;
1-(2-ethynylthiazol-4-yl)-3-((3′-(1-hydroxycyclobutyl)-[1,1′-biphenyl]-4-yl)-methyl)urea A170;
1-(4-(2-(3,3-difluoroazetidin-1-yl)-4-hydroxyquinazolin-5-yl)benzyl)-3-(2-ethynyl-thiazol-4-yl)urea A171;
1-(2-ethynylthiazol-4-yl)-3-(4-(4-hydroxy-2-(2-hydroxyethoxy)quinazolin-5-yl)-benzyl)-urea A172;
1-(2-ethynylthiazol-4-yl)-3-(4-(4-(pyrrolidin-1-yl)pyridin-2-yl)benzyl)urea A173;
1-(2-ethynylthiazol-4-yl)-3-(4-(5-(pyrrolidin-1-yl)pyridin-3-yl)benzyl)urea A174;
1-(2-ethynylthiazol-4-yl)-3-(4-(2-(pyrrolidin-1-yl)pyridin-4-yl)benzyl)urea A175;
1-(2-ethynylthiazol-4-yl)-3-(4-(6-(pyrrolidin-1-yl)pyridin-2-yl)benzyl)urea A176;
1-(4-(benzo[d]thiazol-7-yl)-2-cyanobenzyl)-3-(2-ethynylthiazol-4-yl)urea A177;
1-(4-(benzo[d]thiazol-7-yl)-2-cyanobenzyl)-3-(2-ethynylthiazol-4-yl)urea A178;
1-((5-(benzo[d]thiazol-7-yl)pyridin-2-yl)methyl)-3-(2-ethynylthiazol-4-yl)urea A179;
1-((6-(3-(1-cyanocyclopropyl)phenyl)pyridin-3-yl)methyl)-3-(2-ethynylthiazol-4-yl)urea A180;
(R)-1-(1-(3′-(3,3-difluoropyrrolidin-1-yl)-[1,1′-biphenyl]-4-yl)-2-hydroxyethyl)-3-(2-ethynylthiazol-4-yl)urea A181;
(R)-1-(1-(4-cyclohexylphenyl)-2-hydroxyethyl)-3-(2-ethynylthiazol-4-yl)urea A182;
(R)-1-(2-ethynylthiazol-4-yl)-3-(2-hydroxy-1-(2′,3′,4′,5′-tetrahydro-[1,1′-biphenyl]-4-yl)-ethyl)urea A183;
(R)-1-(2-ethynylthiazol-4-yl)-3-(2-hydroxy-1-(4-(1-methylpiperidin-4-yl)phenyl)-ethyl)-urea A184;
(R)-1-(2-ethynylthiazol-4-yl)-3-(2-hydroxy-1-(4-(1-methyl-1,2,3,6-tetrahydro-pyridin-4-yl)phenyl)ethyl)urea A185;
(R)-1-(2-ethynylthiazol-4-yl)-3-(2-hydroxy-1-(3′-(methylsulfonyl)-[1,1′-biphenyl]-4-yl)ethyl)urea A186;
(R)-1-(1-(3′-(1-cyanocyclopropyl)-[1,1′-biphenyl]-4-yl)-2-hydroxyethyl)-3-(2-ethynyl-thiazol-4-yl)urea A187;
1-((1R)-1-(3′-(2,2-difluorocyclopropyl)-[1,1′-biphenyl]-4-yl)-2-hydroxyethyl)-3-(2-ethynylthiazol-4-yl)urea A188;
(R)-1-(2-ethynylthiazol-4-yl)-3-(2-hydroxy-1-(3′-(1-hydroxycyclobutyl)-[1,1′-biphenyl]-4-yl)ethyl)urea A189;
(R)-1-(1-(3′-(azetidin-1-yl)-[1,1-biphenyl]-4-yl)-2-hydroxyethyl)-3-(2-ethynyl-thiazol-4-yl)urea A190;
(R)-1-(1-(3′-(3,3-difluoroazetidin-1-yl)-[1,1′-biphenyl]-4-yl)-2-hydroxyethyl)-3-(2-ethynylthiazol-4-yl)urea A191;
1-(2-ethynylthiazol-4-yl)-3-((1R)-2-hydroxy-1-(4-(1-methylpiperidin-3-yl)-phenyl)ethyl)-urea A192;
(R)-1-(2-ethynylthiazol-4-yl)-3-(2-hydroxy-1-(4-(1-methyl-1,2,5,6-tetrahydro-pyridin-3-yl)phenyl)ethyl)urea A193;
1-((1R)-1-(4-(1-acetylpiperidin-3-yl)phenyl)-2-hydroxyethyl)-3-(2-ethynyl-thiazol-4-yl)-urea A194;
(R)-1-(2-ethynylthiazol-4-yl)-3-(2-hydroxy-1-(4-(piperidin-1-yl)phenyl)ethyl)-urea A195;
(S)-1-(1-(4-(benzo[d]thiazol-7-yl)phenyl)-2-cyanoethyl)-3-(2-ethynylthiazol-4-yl)urea A196;
(R)-1-(1-(4-(benzo[d]thiazol-7-yl)phenyl)-2-(methylsulfonyl)ethyl)-3-(2-ethynyl-thiazol-4-yl)urea A197;
(R)-2-(4-(benzo[d]thiazol-7-yl)phenyl)-2-(3-(2-ethynylthiazol-4-yl)ureido)-ethane-1-sulfonamide A198;
(R)-2-(4-(benzo[d]thiazol-7-yl)phenyl)-2-(3-(2-ethynylthiazol-4-yl)ureido)-N-methyl-ethane-1-sulfonamide A199;
(R)-1-(1-(4-(benzo[d]thiazol-7-yl)phenyl)-2-methoxyethyl)-3-(2-ethynylthiazol-4-yl)urea A200;
(R)-2-(4-(benzo[d]thiazol-7-yl)phenyl)-2-(3-(2-ethynylthiazol-4-yl)ureido)ethyl acetate A201;
(R)-2-(4-(benzo[d]thiazol-7-yl)phenyl)-2-(3-(2-ethynylthiazol-4-yl)ureido)ethyl pivalate A202;
(R)-1-(1-(4-(benzo[d]thiazol-7-yl)phenyl)-2-hydroxy-2-methylpropyl)-3-(2-ethynyl-thiazol-4-yl)urea A203;
1-((1R)-1-(4-(benzo[d]thiazol-7-yl)phenyl)-2-hydroxypropyl)-3-(2-ethynyl-thiazol-4-yl)-urea A204;
1-(4-(benzo[d]thiazol-7-yl)benzyl)-1-(2-cyanoethyl)-3-(2-ethynylthiazol-4-yl)urea A205;
1-(4-(benzo[d]thiazol-7-yl)benzyl)-3-(2-ethynylthiazol-4-yl)-1-(2-hydroxyethyl)-urea A206;
1-(4-(benzo[d]thiazol-7-yl)benzyl)-3-(2-ethynylthiazol-4-yl)-1-(2-(methylsulfonyl)-ethyl)urea A207;
1-(4-(benzo[d]thiazol-7-yl)benzyl)-1-(cyanomethyl)-3-(2-ethynylthiazol-4-yl)urea A208;
2-(4-(benzo[d]thiazol-7-yl)phenyl)-2-(3-(2-ethynylthiazol-4-yl)ureido)acetamide A209;
(R)-2-(4-(benzo[d]thiazol-7-yl)phenyl)-2-(3-(2-ethynylthiazol-4-yl)ureido)-acetamide A210;
(S)-2-(4-(benzo[d]thiazol-7-yl)phenyl)-2-(3-(2-ethynylthiazol-4-yl)ureido)-acetamide A211;
(R)-2-(3′-(1-cyanocyclopropyl)-[1,1′-biphenyl]-4-yl)-2-(3-(2-ethynylthiazol-4-yl)ureido)-acetamide A212;
(R)-2-(3-(2-ethynylthiazol-4-yl)ureido)-N-methyl-2-(4-(4-oxo-3,4-dihydro-quinazolin-5-yl)phenyl)acetamide A213;
2-(4-(benzo[d]thiazol-7-yl)phenyl)-2-(3-(2-ethynylthiazol-4-yl)ureido)-N-methyl-acetamide A214;
(R)-2-(4-(benzo[d]thiazol-7-yl)phenyl)-2-(3-(2-ethynylthiazol-4-yl)ureido)-N-methyl-acetamide A215;
(S)-2-(4-(benzo[d]thiazol-7-yl)phenyl)-2-(3-(2-ethynylthiazol-4-yl)ureido)-N-methyl-acetamide A216;
(R)-2-(3′-(1-cyanocyclopropyl)-[1,1′-biphenyl]-4-yl)-2-(3-(2-ethynylthiazol-4-yl)ureido)-N-methylacetamide A217;
(R)-2-(3-(2-ethynylthiazol-4-yl)ureido)-2-(3′-(1-hydroxycyclopropyl)-[1,1′-biphenyl]-4-yl)-N-methylacetamide A218;
(R)-2-(3-(2-ethynylthiazol-4-yl)ureido)-N-methyl-2-(3′-propionyl-[1,1′-biphenyl]-4-yl)-acetamide A219;
(R)-2-(4-(benzo[d]thiazol-7-yl)phenyl)-2-(3-(2-ethynylthiazol-4-yl)ureido)-N,N-dimethylacetamide A220;
(R)-2-(3′-cyano-[1,1′-biphenyl]-4-yl)-2-(3-(2-ethynylthiazol-4-yl)ureido)ethyl carbamate A221;
(R)-2-(3′-(1-cyanocyclopropyl)-[1,1′-biphenyl]-4-yl)-2-(3-(2-ethynylthiazol-4-yl)ureido)-ethyl carbonate A222;
(R)-2-(4-(benzo[d]thiazol-7-yl)phenyl)-2-(3-(2-ethynylthiazol-4-yl)ureido)ethyl carbamate A223;
(R)-2-(4-(benzo[d]thiazol-7-yl)phenyl)-2-(3-(2-ethynylthiazol-4-yl)ureido)ethyl methyl-carbamate A224;
(R)-2-(4-(benzo[d][1,2,3]thiadiazol-7-yl)phenyl)-2-(3-(2-ethynylthiazol-4-yl)-ureido)-ethyl carbamate A225;
(R)-2-(4-(benzo[c][1,2,5]thiadiazol-4-yl)phenyl)-2-(3-(2-ethynylthiazol-4-yl)-ureido)-ethyl carbamate A226;
(R)-2-(3-(2-ethynylthiazol-4-yl)ureido)-2-(4-(quinolin-8-yl)phenyl)ethyl carbamate A227;
(R)-2-(3-(2-ethynylthiazol-4-yl)ureido)-2-(4-(isoquinolin-8-yl)phenyl)ethyl carbamate A228;
(R)-2-(3-(2-ethynylthiazol-4-yl)ureido)-2-(4-(isoquinolin-5-yl)phenyl)ethyl carbamate A229;
(R)-2-(3-(2-ethynylthiazol-4-yl)ureido)-2-(4-(quinolin-5-yl)phenyl)ethyl carbamate A230;
(R)-2-(3-(2-ethynylthiazol-4-yl)ureido)-2-(4-(1-methyl-1H-indazol-4-yl)phenyl)-ethyl carbamate A231;
(2R)-2-(3-(2-ethynylthiazol-4-yl)ureido)-2-(4-(1-methyl-4,5,6,7-tetrahydro-1H-indazol-4-yl)phenyl)ethyl carbamate A232;
(R)-2-(3-(2-ethynylthiazol-4-yl)ureido)-2-(4-(1-methyl-6,7-dihydro-1H-indazol-4-yl)-phenyl)ethyl carbamate A233;
(R)-2-(3-(2-ethynylthiazol-4-yl)ureido)-2-(4-(6-(pyrrolidin-1-yl)pyridin-2-yl)-phenyl)-ethyl carbamate A234;
(R)-2-(3-(2-ethynylthiazol-4-yl)ureido)-2-(4-(2-(pyrrolidin-1-yl)pyridin-4-yl)-phenyl)-ethyl carbamate A235;
(R)-2-(5-(3-(1-cyanocyclopropyl)phenyl)pyridin-2-yl)-2-(3-(2-ethynylthiazol-4-yl)-ureido)ethyl carbamate A236;
1-(1-(4-(benzo[d]thiazol-7-yl)phenyl)piperidin-4-yl)-3-(2-ethynylthiazol-4-yl)urea A237;
1-((4-(3-(1-cyanocyclopropyl)phenyl)cyclohexyl)methyl)-3-(2-ethynylthiazol-4-yl)urea A238;
1-((3′-(1-cyanocyclopropyl)-2,3,4,5-tetrahydro-[1,1′-biphenyl]-4-yl)methyl)-3-(2-ethynylthiazol-4-yl)urea A239;
1-((1-(3-(1-cyanocyclopropyl)phenyl)piperidin-4-yl)methyl)-3-(2-ethynylthiazol-4-yl)-urea A240;
(R)-1-(1-(4-(benzo[d]thiazol-7-yl)phenyl)-2-hydroxyethyl)-3-(2-ethynyl-pyrimidin-4-yl)-urea A241;
2-(4-(benzo[d]thiazol-7-yl)phenyl)-N-(2-ethynylthiazol-4-yl)pyrrolidine-1-carboxamide B1;
3-(4-(benzo[d]thiazol-7-yl)phenyl)-N-(2-ethynylthiazol-4-yl)azetidine-1-carboxamide B2;
6-(benzo[d]thiazol-7-yl)-N-(2-ethynylthiazol-4-yl)-3,4-dihydroisoquinoline-2(1H)-carboxamide B3;
5-(benzo[d]thiazol-7-yl)-N-(2-ethynylthiazol-4-yl)isoindoline-2-carboxamide B2;
4-(4-(benzo[d]thiazol-7-yl)phenyl)-N-(2-ethynylthiazol-4-yl)piperazine-1-carboxamide B5;
4-(4-(benzo[d]thiazol-7-yl)phenyl)-N-(2-ethynylthiazol-4-yl)piperidine-1-carboxamide B6;
N-(2-ethynylthiazol-4-yl)-4-(5-(3-(2-oxooxazolidin-3-yl)phenyl)pyridin-2-yl)piperazine-1-carboxamide B7;
N-(2-ethynylthiazol-4-yl)-4-(3′-(oxetan-3-ylamino)-[1,1′-biphenyl]-4-yl)piperazine-1-carboxamide B8;
N-(2-ethynylthiazol-4-yl)-4-(3′-((3-hydroxycyclobutyl)amino)-[1,1′-biphenyl]-4-yl)-piperazine-1-carboxamide B9;
N-(2-ethynylthiazol-4-yl)-4-(3′-(3-hydroxyazetidin-1-yl)-[1,1′-biphenyl]-4-yl)piperazine-1-carboxamide B10;
N-(2-ethynylthiazol-4-yl)-4-(4-(imidazo[1,5-a]pyridin-5-yl)phenyl)piperazine-1-carboxamide B11;
4-(4-([1,2,4]triazolo[4,3-a]pyridin-5-yl)phenyl)-N-(2-ethynylthiazol-4-yl)piperazine-1-carboxamide B12;
N-(2-ethynylthiazol-4-yl)-4-(4-(imidazo[1,5-a]pyridin-8-yl)phenyl)piperazine-1-carboxamide B13;
4-(4-([1,2,3]triazolo[1,5-a]pyridin-4-yl)phenyl)-N-(2-ethynylthiazol-4-yl)piperazine-1-carboxamide B14;
N-(2-ethynylthiazol-4-yl)-4-(3′-(pyrrolidin-1-yl)-[1,1′-biphenyl]-4-yl)piperazine-1-carboxamide B15;
(S)—N-(2-ethynylthiazol-4-yl)-4-(3′-(3-hydroxypyrrolidin-1-yl)-[1,1′-biphenyl]-4-yl)-piperazine-1-carboxamide B16;
4-(4-(1,1-dioxido-3-oxo-2,3-dihydrobenzo[b]thiophen-7-yl)phenyl)-N-(2-ethynylthiazol-4-yl)piperazine-1-carboxamide B17;
N-(2-ethynylthiazol-4-yl)-4-(4-(1-methyl-1H-indazol-4-yl)phenyl)piperazine-1-carboxamide B18;
N-(2-ethynylthiazol-4-yl)-4-(3′-(pyrrolidin-1-yl)-[1,1′-biphenyl]-4-yl)piperazine-1-carboxamide B19;
N-(2-ethynylthiazol-4-yl)-4-(4-(1-oxo-1,2-dihydroisoquinolin-8-yl)phenyl)piperazine-1-carboxamide B20;
N-(2-ethynylthiazol-4-yl)-4-(4-(3-hydroxy-1,1-dioxido-2,3-dihydrobenzo[b]thiophen-7-yl)phenyl)piperazine-1-carboxamide B21;
N-(2-ethynylthiazol-4-yl)-4-(4-(4-oxo-3,4-dihydroquinazolin-5-yl)phenyl)piperazine-1-carboxamide B22;
(S)-4-(4-(benzo[d]thiazol-7-yl)phenyl)-N-(2-ethynylthiazol-4-yl)-2-(hydroxymethyl)-piperazine-1-carboxamide B23;
(R)-4-(4-(benzo[d]thiazol-7-yl)phenyl)-N-(2-ethynylthiazol-4-yl)-2-(hydroxymethyl)-piperazine-1-carboxamide B24;
4-(4-(2-amino-6-cyanobenzo[d]thiazol-7-yl)phenyl)-N-(2-ethynylthiazol-4-yl)piperazine-1-carboxamide B25;
4-(4-(6-cyanobenzo[d]thiazol-7-yl)phenyl)-N-(2-ethynylthiazol-4-yl)piperazine-1-carboxamide B26;
4-(4-(2-aminobenzo[d]thiazol-7-yl)phenyl)-N-(2-ethynylthiazol-4-yl)piperazine-1-carboxamide B27;
4-(4-(benzo[d][1,2,3]thiadiazol-7-yl)phenyl)-N-(2-ethynylthiazol-4-yl)piperazine-1-carboxamide B28;
4-(4-([1,2,4]triazolo[1,5-a]pyridin-8-yl)phenyl)-N-(2-ethynylthiazol-4-yl)piperazine-1-carboxamide B29;
4-(4-([1,2,4]triazolo[4,3-a]pyridin-8-yl)phenyl)-N-(2-ethynylthiazol-4-yl)piperazine-1-carboxamide B30;
4-(2′-cyano-[1,1′-biphenyl]-4-yl)-N-(2-ethynylthiazol-4-yl)piperazine-1-carboxamide B31;
4-(4-([1,2,4]triazolo[4,3-a]pyridin-5-yl)phenyl)-N-(2-ethynylthiazol-4-yl)piperazine-1-carboxamide B32;
N-(2-ethynylthiazol-4-yl)-4-(4-(imidazo[1,2-a]pyridin-5-yl)phenyl)piperazine-1-carboxamide B33;
4-(4-([1,2,4]triazolo[1,5-a]pyridin-5-yl)phenyl)-N-(2-ethynylthiazol-4-yl)piperazine-1-carboxamide B34;
N-(2-ethynylthiazol-4-yl)-4-(4-(imidazo[1,2-a]pyridin-8-yl)phenyl)piperazine-1-carboxamide B35;
(R)-4-(4-(benzo[d]thiazol-7-yl)phenyl)-N-(2-ethynylthiazol-4-yl)-3-(hydroxymethyl)-piperazine-1-carboxamide B36;
(S)-4-(4-(benzo[d]thiazol-7-yl)phenyl)-N-(2-ethynylthiazol-4-yl)-3-(hydroxymethyl)-piperazine-1-carboxamide B37;
4-(3′-(1-cyanocyclopropyl)-[1,1′-biphenyl]-4-yl)-N-(2-ethynylthiazol-4-yl)piperazine-1-carboxamide B38;
4-(3′-cyano-[1,1′-biphenyl]-4-yl)-N-(2-ethynylthiazol-4-yl)piperazine-1-carboxamide B39;
4-(4-(benzo[d]thiazol-7-yl)-2-cyanophenyl)-N-(2-ethynylthiazol-4-yl)piperazine-1-carboxamide B40;
4-(4-(benzo[d]thiazol-7-yl)-3-cyanophenyl)-N-(2-ethynylthiazol-4-yl)piperazine-1-carboxamide B41;
4-(3′-cyclopropyl-[1,1′-biphenyl]-4-yl)-N-(2-ethynylthiazol-4-yl)piperazine-1-carboxamide B42;
N-(2-ethynylthiazol-4-yl)-4-(3′-(2-oxopyrrolidin-1-yl)-[1,1′-biphenyl]-4-yl)piperazine-1-carboxamide B43;
4-((4-(benzo[d]thiazol-7-yl)phenyl)amino)-N-(2-ethynylthiazol-4-yl)piperidine-1-carboxamide B44;
4-(4-(2-amino-[1,2,4]triazolo[1,5-a]pyridin-5-yl)phenyl)-N-(2-ethynylthiazol-4-yl)-piperazine-1-carboxamide B45;
4-(3′-(cyclopent-1-en-1-yl)-[1,1′-biphenyl]-4-yl)-N-(2-ethynylthiazol-4-yl)piperazine-1-carboxamide B46;
N-(2-ethynylthiazol-4-yl)-4-(3′-(2-oxoimidazolidin-1-yl)-[1,1′-biphenyl]-4-yl)piperazine-1-carboxamide B47;
N-(2-ethynylthiazol-4-yl)-4-(3′-(1-hydroxycyclopentyl)-[1,1′-biphenyl]-4-yl)piperazine-1-carboxamide B48;
N-(2-ethynylthiazol-4-yl)-4-(3′-(3-hydroxyazetidin-1-yl)-[1,1′-biphenyl]-4-yl)piperazine-1-carboxamide B49;
N-(2-ethynylthiazol-4-yl)-4-(3′-(3-hydroxypyrrolidin-1-yl)-[1,1′-biphenyl]-4-yl)-piperazine-1-carboxamide B50;
4-(3′-(azetidin-1-yl)-[1,1′-biphenyl]-4-yl)-N-(2-ethynylthiazol-4-yl)piperazine-1-carboxamide B51;
(S)—N-(2-ethynylthiazol-4-yl)-2-(hydroxymethyl)-4-(3′-(pyrrolidin-1-yl)-[1,1′-biphenyl]-4-yl)piperazine-1-carboxamide B52;
(R)-4-(3-cyano-3′-(pyrrolidin-1-yl)-[1,1′-biphenyl]-4-yl)-N-(2-ethynylthiazol-4-yl)-2-(hydroxymethyl)piperazine-1-carboxamide B53;
(R)—N-(2-ethynylthiazol-4-yl)-2-(hydroxymethyl)-4-(3′-(pyrrolidin-1-yl)-[1,1′-biphenyl]-4-yl)piperazine-1-carboxamide B54;
(R)-4-(4-(benzo[d]thiazol-7-yl)phenyl)-N-(2-ethynylthiazol-4-yl)-2-(hydroxymethyl)-piperazine-1-carboxamide B55;
(R)—N-(2-ethynylthiazol-4-yl)-2-(hydroxymethyl)-4-(5-(3-(pyrrolidin-1-yl)phenyl)pyridin-2-yl)piperazine-1-carboxamide B56;
N-(2-ethynylthiazol-4-yl)-4-(5-(3-(2-oxooxazolidin-3-yl)phenyl)pyridin-2-yl)piperazine-1-carboxamide B57;
(R)-4-(5-(benzo[d]thiazol-7-yl)pyridin-2-yl)-N-(2-ethynylthiazol-4-yl)-2-(hydroxymethyl)piperazine-1-carboxamide B58;
(R)-4-(5-(benzo[d]thiazol-7-yl)-3-cyanopyridin-2-yl)-N-(2-ethynylthiazol-4-yl)-2-(hydroxymethyl)piperazine-1-carboxamide B59;
2-ethynyl-N-(4-(6-fluorobenzo[d]thiazol-5-yl)phenethyl)thiazole-4-carboxamide C1;
N-(4-(benzo[d]thiazol-7-yl)-3-fluorophenethyl)-2-ethynylthiazole-4-carboxamide C2;
7-(4-(2-(2-ethynylthiazole-4-carboxamido)ethyl)phenyl)benzo[d]thiazole-6-carboxamide C3;
2-ethynyl-N-(2-(3′-(methylcarbamoyl)-[1,1′-biphenyl]-4-yl)ethyl)thiazole-4-carboxamide C4;
2-ethynyl-N-(4-(6-fluorobenzo[d]thiazol-7-yl)phenethyl)thiazole-4-carboxamide C5;
N-(2-(2′-(N,N-dimethylsulfamoyl)-[1,1′-biphenyl]-4-yl)ethyl)-2-ethynylthiazole-4-carboxamide C6;
methyl 2-(2-ethynylthiazole-4-carboxamido)-5-(4-(2-(2-ethynylthiazole-4-carboxamido)-ethyl)phenyl)benzo[d]thiazole-7-carboxylate C7;
7-(4-(2-(2-ethynylthiazole-4-carboxamido)ethyl)phenyl)benzo[d]thiazole-5-carboxamide C8;
7-(4-(2-(2-ethynylthiazole-4-carboxamido)ethyl)phenyl)-N-methylbenzo[d]thiazole-5-carboxamide C9;
methyl 7-(4-(2-(2-ethynylthiazole-4-carboxamido)ethyl)phenyl)benzo[d]thiazole-5-carboxylate C10;
2-ethynyl-N-(4-(quinoxalin-5-yl)phenethyl)thiazole-4-carboxamide C11;
2-ethynyl-N-(2-(2′-(methylcarbamoyl)-[1,1′-biphenyl]-4-yl)ethyl)thiazole-4-carboxamide C12;
2-ethynyl-N-(4-(3-oxoisoindolin-4-yl)phenethyl)thiazole-4-carboxamide C13;
N-(4-(2,3-dioxoindolin-4-yl)phenethyl)-2-ethynylthiazole-4-carboxamide C14;
2-ethynyl-N-(2-(2′-(methylsulfonyl)-[1,1′-biphenyl]-4-yl)ethyl)thiazole-4-carboxamide C15;
2-ethynyl-N-(4-(1-hydroxyisoquinolin-8-yl)phenethyl)thiazole-4-carboxamide C16;
2-ethynyl-N-(4-(1-oxoisoindolin-4-yl)phenethyl)thiazole-4-carboxamide C17;
2-ethynyl-N-(4-(3-oxo-2,3-dihydro-1H-inden-4-yl)phenethyl)thiazole-4-carboxamide C18;
2-ethynyl-N-(4-(thiazolo[4,5-c]pyridin-7-yl)phenethyl)thiazole-4-carboxamide C19′;
2-ethynyl-N-(2-(2′-(N-methylsulfamoyl)-[1,1′-biphenyl]-4-yl)ethyl)thiazole-4-carboxamide C20;
2-ethynyl-N-(4-(isoquinolin-5-yl)phenethyl)thiazole-4-carboxamide C21;
2-ethynyl-N-(4-(2-methyl-1-oxoisoindolin-4-yl)phenethyl)thiazole-4-carboxamide C22;
2-ethynyl-N-(4-(quinolin-5-yl)phenethyl)thiazole-4-carboxamide C23;
2-ethynyl-N-(4-(quinazolin-5-yl)phenethyl)thiazole-4-carboxamide C24;
N-(4-(benzo[d]thiazol-5-yl)phenethyl)-2-ethynylthiazole-4-carboxamide C25;
2-ethynyl-N-(3-(1-methyl-2-oxoindolin-4-yl)phenethyl)thiazole-4-carboxamide C26;
2-ethynyl-N-(3-(2-oxoindolin-4-yl)phenethyl)thiazole-4-carboxamide C27;
N-(4-(1H-indol-4-yl)benzyl)-2-ethynylthiazole-4-carboxamide C28;
2-ethynyl-N-(3-(2-methyl-2H-indazol-4-yl)phenethyl)thiazole-4-carboxamide C29;
2-ethynyl-N-(3-(1-methyl-1H-indazol-4-yl)phenethyl)thiazole-4-carboxamide C30;
N-(4-(benzo[d]thiazol-4-yl)phenethyl)-2-ethynylthiazole-4-carboxamide C31;
2-ethynyl-N-(4-(2-methyl-2H-indazol-4-yl)phenethyl)thiazole-4-carboxamide C32;
2-ethynyl-N-(4-(1-methyl-1H-indazol-4-yl)phenethyl)thiazole-4-carboxamide C33;
N-(4-(benzo[d]thiazol-7-yl)phenethyl)-2-ethynylthiazole-4-carboxamide C34;
2-ethynyl-N-(3-(1-methyl-1H-indol-4-yl)phenethyl)thiazole-4-carboxamide C35;
N-(3-(1H-indol-4-yl)phenethyl)-2-ethynylthiazole-4-carboxamide C36;
2-ethynyl-N-(4-(2-oxoindolin-4-yl)benzyl)thiazole-4-carboxamide C37;
2-ethynyl-N-(3-(pyridin-3-yl)phenethyl)thiazole-4-carboxamide C38;
N-(3-(1H-indazol-4-yl)phenethyl)-2-ethynylthiazole-4-carboxamide C39;
N-(4-(1H-indazol-4-yl)benzyl)-2-ethynylthiazole-4-carboxamide C40;
2-ethynyl-N-(4-(1-methyl-1H-pyrazol-3-yl)phenethyl)thiazole-4-carboxamide C41;
N-(4-(benzo[d]thiazol-6-yl)phenethyl)-2-ethynylthiazole-4-carboxamide C42;
2-ethynyl-N-(4-(1-methyl-2-oxoindolin-4-yl)phenethyl)thiazole-4-carboxamide C43;
2-ethynyl-N-(quinolin-2-ylmethyl)thiazole-4-carboxamide C44;
N-(2-cyanophenethyl)-2-ethynylthiazole-4-carboxamide C45;
methyl 2-(3-((2-ethynylthiazole-4-carboxamido)methyl)phenyl)acetate C46;
N-(3-(2-amino-2-oxoethyl)benzyl)-2-ethynylthiazole-4-carboxamide C47;
2-ethynyl-N-(4-(2-oxoindolin-4-yl)phenethyl)thiazole-4-carboxamide C48;
N-(4-(1H-indazol-4-yl)phenethyl)-2-ethynylthiazole-4-carboxamide C49;
2-ethynyl-N-(4-(1-methyl-1H-indol-4-yl)phenethyl)thiazole-4-carboxamide C50;
N-(4-(1H-indol-4-yl)phenethyl)-2-ethynylthiazole-4-carboxamide C51;
N-(3-chlorobenzyl)-2-ethynyl-5-methylthiazole-4-carboxamide C52;
2-ethynyl-N-(4-(thiazol-4-yl)phenethyl)thiazole-4-carboxamide C53;
2-ethynyl-N-(4-(1-methyl-1H-pyrazol-3-yl)benzyl)thiazole-4-carboxamide C54;
2-ethynyl-N-(4-(thiazol-4-yl)benzyl)thiazole-4-carboxamide C55;
N-(3-chlorobenzyl)-2-ethynyl-5-phenylthiazole-4-carboxamide C56;
2-ethynyl-N-(4-(thiazol-5-yl)phenethyl)thiazole-4-carboxamide C57;
N-(3-(1H-pyrazol-4-yl)benzyl)-2-ethynylthiazole-4-carboxamide C58;
2-ethynyl-N-(3-(1-methyl-1H-pyrazol-4-yl)benzyl)thiazole-4-carboxamide C59;
N-(3-(1H-pyrazol-3-yl)benzyl)-2-ethynylthiazole-4-carboxamide C60;
2-ethynyl-N-(4-(thiazol-5-yl)benzyl)thiazole-4-carboxamide C61;
N-(2,3-dihydro-1H-inden-2-yl)-2-ethynylthiazole-4-carboxamide C62;
N-(2-cyanobenzyl)-2-ethynylthiazole-4-carboxamide C63;
2-ethynyl-N-(naphthalen-2-ylmethyl)thiazole-4-carboxamide C64;
2-ethynyl-N-(3-(pyridin-3-yl)benzyl)thiazole-4-carboxamide C65;
N-(benzo[d]thiazol-6-ylmethyl)-2-ethynylthiazole-4-carboxamide C66;
(R)—N-(1-(3-chlorophenyl)ethyl)-2-ethynylthiazole-4-carboxamide C67;
N-(1-(3-chlorophenyl)cyclopropyl)-2-ethynylthiazole-4-carboxamide C68;
(S)—N-(1-(3-chlorophenyl)ethyl)-2-ethynylthiazole-4-carboxamide C69;
2-ethynyl-N-(2-(hydroxymethyl)benzyl)thiazole-4-carboxamide C70;
(S)—N-(2,3-dihydro-1H-inden-1-yl)-2-ethynylthiazole-4-carboxamide C71;
(R)—N-(2,3-dihydro-1H-inden-1-yl)-2-ethynylthiazole-4-carboxamide C72;
methyl 3-(4-((2-ethynylthiazole-4-carboxamido)methyl)phenyl)propanoate C73;
methyl 2-((2-ethynylthiazole-4-carboxamido)methyl)benzoate C74;
2-ethynyl-N-(4-(1-methyl-1H-pyrazol-4-yl)phenethyl)thiazole-4-carboxamide C75;
N-(4-(1H-pyrazol-4-yl)phenethyl)-2-ethynylthiazole-4-carboxamide C76;
N-(2-ethynylthiazol-4-yl)-2-phenylacetamide C77;
2-ethynyl-N-(1-phenylpiperidin-4-yl)thiazole-4-carboxamide C78;
2-ethynyl-N-(4-(pyridin-3-yl)phenethyl)thiazole-4-carboxamide C79;
N-(4-bromophenethyl)-2-ethynylthiazole-4-carboxamide C80;
2-ethynyl-N-(3-(methylsulfonamido)phenethyl)thiazole-4-carboxamide C81;
N-(3-acetamidophenethyl)-2-ethynylthiazole-4-carboxamide C82;
2-ethynyl-N-(4-(1-methyl-1H-pyrazol-4-yl)benzyl)thiazole-4-carboxamide C83;
methyl 4-(2-(2-ethynylthiazole-4-carboxamido)ethyl)benzoate C84;
N-(3-aminophenethyl)-2-ethynylthiazole-4-carboxamide C85;
N-((2,3-dihydrobenzo[b][1,4]dioxin-6-yl)methyl)-2-ethynylthiazole-4-carboxamide C86;
2-ethynyl-N-(3-nitrophenethyl)thiazole-4-carboxamide C87;
N-(4-acetamidophenethyl)-2-ethynylthiazole-4-carboxamide C88;
N-(4-acetamidobenzyl)-2-ethynylthiazole-4-carboxamide C89;
N-benzyl-2-cyanothiazole-4-carboxamide C90;
N-(4-(1H-pyrazol-3-yl)benzyl)-2-ethynylthiazole-4-carboxamide C91;
N-(4-(1H-pyrazol-4-yl)benzyl)-2-ethynylthiazole-4-carboxamide C92;
2-ethynyl-N-(4-(pyridin-3-yl)benzyl)thiazole-4-carboxamide C93;
N-(3-acetamidobenzyl)-2-ethynylthiazole-4-carboxamide C94;
N-(3-aminobenzyl)-2-ethynylthiazole-4-carboxamide C95;
2-ethynyl-N-(quinolin-6-ylmethyl)thiazole-4-carboxamide C96;
methyl 3-(2-(2-ethynylthiazole-4-carboxamido)ethyl)benzoate C97;
N-benzyl-2-ethynyl-1-methyl-1H-imidazole-4-carboxamide C98;
2-ethynyl-N-(4-(methylsulfonamido)phenethyl)thiazole-4-carboxamide C99;
2-ethynyl-N-methyl-N-(4-nitrophenethyl)thiazole-4-carboxamide C100;
2-ethynyl-N-(4-(methylsulfonyl)phenethyl)thiazole-4-carboxamide C101;
N-(4-cyanophenethyl)-2-ethynylthiazole-4-carboxamide C102;
2-ethynyl-N-(4-nitrobenzyl)thiazole-4-carboxamide C103;
N-(4-cyanobenzyl)-2-ethynylthiazole-4-carboxamide C104;
2-ethynyl-N-(3-(methylsulfonyl)phenethyl)thiazole-4-carboxamide C105;
N-(2-chlorophenethyl)-2-ethynylthiazole-4-carboxamide C106;
2-ethynyl-N-(3-nitrobenzyl)thiazole-4-carboxamide C107;
2-ethynyl-N-(4-(methylsulfonyl)benzyl)thiazole-4-carboxamide C108;
N-((3-chloropyridin-2-yl)methyl)-2-ethynylthiazole-4-carboxamide C109;
N-(3-chlorophenethyl)-2-ethynylthiazole-4-carboxamide C110;
N-benzyl-2-ethynyl-1H-imidazole-4-carboxamide C111;
N-([1,1′-biphenyl]-2-ylmethyl)-2-ethynylthiazole-4-carboxamide C112;
2-ethynyl-N-(naphthalen-1-ylmethyl)thiazole-4-carboxamide C113;
N-([1,1′-biphenyl]-4-ylmethyl)-2-ethynylthiazole-4-carboxamide C114;
N-([1,1′-biphenyl]-3-ylmethyl)-2-ethynylthiazole-4-carboxamide C115;
ethyl 3-(N-benzyl-2-ethynylthiazole-4-carboxamido)propanoate C116;
methyl 4-((2-ethynylthiazole-4-carboxamido)methyl)benzoate C117;
N-(4-aminobenzyl)-2-ethynylthiazole-4-carboxamide C118;
2-ethynyl-N-((1-methyl-1H-pyrazol-3-yl)methyl)thiazole-4-carboxamide C119;
2-ethynyl-N-((1-methyl-1H-pyrazol-4-yl)methyl)thiazole-4-carboxamide C120;
2-ethynyl-N-(4-hydroxybenzyl)thiazole-4-carboxamide C121;
2-ethynyl-N-(4-hydroxy-3-methoxyphenethyl)thiazole-4-carboxamide C122;
N-(2-ethynylthiazol-4-yl)-3-phenylpropanamide C123;
methyl 3-((2-ethynylthiazole-4-carboxamido)methyl)benzoate C124;
N-(3-chlorobenzyl)-2-ethynylthiazole-4-carboxamide C125;
2-ethynyl-N-(pyridin-2-ylmethyl)thiazole-4-carboxamide C126;
N-(3-cyanobenzyl)-2-ethynylthiazole-4-carboxamide C127;
N-(4-aminophenethyl)-2-ethynylthiazole-4-carboxamide C128;
2-ethynyl-N-(4-nitrophenethyl)thiazole-4-carboxamide C129;
2-ethynyl-N-(1H-indazol-4-yl)thiazole-4-carboxamide C130;
ethyl N-benzyl-N-(2-ethynylthiazole-4-carbonyl)glycinate C133;
N-benzyl-2-ethynyl-1-methyl-1H-imidazole-5-carboxamide C134;
N-benzyl-2-ethynyl-N-(2-hydroxyethyl)thiazole-4-carboxamide C135;
2-ethynyl-N-(1H-indazol-7-yl)thiazole-4-carboxamide C136;
(S)-2-ethynyl-N-(2-hydroxy-2-phenylethyl)thiazole-4-carboxamide C137;
N-(2-acetamidobenzyl)-2-ethynylthiazole-4-carboxamide C138;
N-benzyl-2-ethynyl-N-methylthiazole-4-carboxamide C139;
2-ethynyl-N-phenethylthiazole-4-carboxamide C140;
N-((1H-indol-4-yl)methyl)-2-ethynylthiazole-4-carboxamide C141;
N-(2-aminobenzyl)-2-ethynylthiazole-4-carboxamide C142;
N-benzyl-2-ethynylthiazole-5-carboxamide C143;
N-benzyl-2-ethynyloxazole-4-carboxamide C144;
N-(2-chlorobenzyl)-2-ethynylthiazole-4-carboxamide C145;
2-ethynyl-N-(2-methoxybenzyl)thiazole-4-carboxamide C146;
N-benzyl-4-ethynylthiazole-2-carboxamide C147;
N-benzyl-2-ethynylpyrimidine-4-carboxamide C148;
N-benzyl-6-ethynylpicolinamide C149;
N-benzyl-2-ethynylthiazole-4-carboxamide C150;
N-benzyl-2-ethynylisonicotinamide C151;
(4-(4-(1,5-dimethyl-1H-indazol-4-yl)phenyl)piperazin-1-yl)(2-ethynylthiazol-4-yl)methanone D1;
methyl 4-(4-(4-(2-ethynylthiazole-4-carbonyl)piperazin-1-yl)phenyl)-1-methyl-1H-indazole-6-carboxylate D2;
7-(4-(4-(2-ethynylthiazole-4-carbonyl)piperazin-1-yl)phenyl)-N-methylbenzo[d]thiazole-5-carboxamide D3;
methyl 7-(4-(4-(2-ethynylthiazole-4-carbonyl)piperazin-1-yl)phenyl)benzo[d]-thiazole-5-carboxylate D4;
7-(4-(4-(2-ethynylthiazole-4-carbonyl)piperazin-1-yl)phenyl)benzo[d]thiazole-5-carboxamide D5;
7-(4-(4-(2-ethynylthiazole-4-carbonyl)piperazin-1-yl)phenyl)-N,N-dimethylbenzo[d]-thiazole-5-carboxamide D6;
(4-(4-(benzo[d]thiazol-7-yl)phenyl)piperazin-1-yl)(2-ethynylthiazol-5-yl)methanone D7;
(4-(4-(benzo[d]thiazol-4-yl)phenyl)piperazin-1-yl)(2-ethynylthiazol-5-yl)methanone D8;
(4-(4-(benzo[d]thiazol-7-yl)-2-chlorophenyl)piperazin-1-yl)(2-ethynylthiazol-4-yl)methanone D9;
(4-(5-(benzo[d]thiazol-7-yl)pyridin-2-yl)piperazin-1-yl)(2-ethynylthiazol-4-yl)methanone D10;
(4-(4-(benzo[d]thiazol-7-yl)phenyl)-4-fluoropiperidin-1-yl)(2-ethynylthiazol-4-yl)methanone D11;
5-(benzo[d]thiazol-7-yl)-2-(4-(2-ethynylthiazole-4-carbonyl)piperazin-1-yl)benzonitrile D12;
(3-(4-(benzo[d]thiazol-7-yl)phenyl)azetidin-1-yl)(2-ethynylthiazol-4-yl)methanone D13;
(4-(4-(benzo[d]thiazol-7-yl)phenyl)-4-hydroxypiperidin-1-yl)(2-ethynylthiazol-4-yl)methanone D14;
(4-(3-(benzo[d]thiazol-7-yl)phenyl)piperidin-1-yl)(2-ethynylthiazol-4-yl)methanone D15;
(4-(3-(benzo[d]thiazol-7-yl)phenyl)piperazin-1-yl)(2-ethynylthiazol-4-yl)methanone D16;
(4-(4-(benzo[d]thiazol-7-yl)phenyl)piperazin-1-yl)(2-ethynylthiazol-4-yl)methanone D17;
(4-(4-(benzo[d]thiazol-7-yl)phenyl)piperidin-1-yl)(2-ethynylthiazol-4-yl)methanone D18;
(2-ethynylthiazol-4-yl)(4-(4-(2-methyl-2H-indazol-4-yl)phenyl)piperidin-1-yl)methanone D19;
(2-ethynylthiazol-4-yl)(4-(4-(1-methyl-1H-indazol-4-yl)phenyl)piperidin-1-yl)methanone D20;
(4-(4-(1H-indazol-4-yl)phenyl)piperidin-1-yl)(2-ethynylthiazol-4-yl)methanone D21;
(2-ethynylthiazol-4-yl)(4-(4-(1-methyl-1H-indazol-4-yl)phenyl)piperazin-1-yl)methanone D22;
(2-ethynylthiazol-4-yl)(4-(4-(2-methyl-2H-indazol-4-yl)phenyl)piperazin-1-yl)methanone D23;
4-(1-(2-ethynylthiazole-4-carbonyl)piperidin-4-yl)benzonitrile D24;
(4-(4-(1H-indazol-4-yl)phenyl)piperazin-1-yl)(2-ethynylthiazol-4-yl)methanone D25;
4-(4-(2-ethynylthiazole-4-carbonyl)piperazin-1-yl)benzonitrile D26;
(2-ethynylthiazol-4-yl)(4-(4-nitrophenyl)piperazin-1-yl)methanone D27;
(2-ethynylthiazol-4-yl)(4-phenylpiperazin-1-yl)methanone D28;
(2-ethynylthiazol-4-yl)(4-phenylpiperidin-1-yl)methanone D29;
(3,4-dihydroisoquinolin-2(1H)-yl)(2-ethynylthiazol-4-yl)methanone D30;
(2-ethynylthiazol-4-yl)(4-hydroxypiperidin-1-yl)methanone D31;
(2-ethynylthiazol-4-yl)(2-(2-hydroxyethyl)piperidin-1-yl)methanone D32;
(2-ethynylthiazol-4-yl)(3-(hydroxymethyl)piperidin-1-yl)methanone D33;
(2-ethynylthiazol-4-yl)(3-hydroxypiperidin-1-yl)methanone D34;
methyl 1-(2-ethynylthiazole-4-carbonyl)piperidine-4-carboxylate D35;
methyl 1-(2-ethynylthiazole-4-carbonyl)piperidine-3-carboxylate D36;
methyl 1-(2-ethynylthiazole-4-carbonyl)piperidine-2-carboxylate D37;
3-(4-(benzo[d]thiazol-7-yl)phenyl)-N-(2-ethynylthiazol-4-yl)propanamide E1;
(S)-2-amino-N-(2-ethynylthiazol-4-yl)-3-phenylpropanamide E2;
(R)-2-amino-N-(2-ethynylthiazol-4-yl)-3-phenylpropanamide E3;
N-(2-ethynylthiazol-4-yl)-3-(4-(1-methyl-1H-indazol-4-yl)phenyl)propanamide E4;
(R)-2-amino-3-(2-cyanophenyl)-N-(2-ethynylthiazol-4-yl)propenamide E5;
2-ethynyl-N-(3-(thiazol-5-yl)phenethyl)thiazole-4-carboxamide E6;
2-ethynyl-N-(3-(thiazol-4-yl)phenethyl)thiazole-4-carboxamide E7;
(S)-2-amino-3-(2-cyanophenyl)-N-(2-ethynylthiazol-4-yl)propenamide E8;
3-(benzo[d][1,3]dioxol-5-yl)-N-(2-ethynylthiazol-4-yl)propanamide E9;
N-(2-ethynylthiazol-4-yl)quinoline-2-carboxamide E10;
N-(2-ethynylthiazol-4-yl)cinnamamide E11; or
N-(2-ethynylthiazol-4-yl)benzamide E12;
or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

117. A pharmaceutical composition comprising the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof; and a pharmaceutically acceptable excipient.

118. A method of treating a proliferative disease in a subject, comprising administering to the subject in need thereof the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

119. A method of treating a disorder, disease, or condition mediated by a glutathione peroxidase 4 in a subject, comprising administering to the subject in need thereof the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

120. The method of claim 119, wherein the disorder, disease, or condition mediated by a glutathione peroxidase is a proliferative disease.

121. The method of claim 118 or 120, wherein the proliferative disease is cancer.

122. The method of any one of claims 118 to 121, wherein the subject is a human.

123. A method of inhibiting the growth of a cell, comprising contacting the cell with an effective amount of the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

124. A method of inducing ferroptosis in a cell, comprising contacting the cell with an effective amount of the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

125. A method of inhibiting irreversibly inhibiting the activity of a protein, comprising contacting the protein with an effective amount of the compound of any one of claims 1 to 116, or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

126. The method of claim 125, wherein the protein is a kinase, GTPase, protease, or glutathione peroxidase.

127. The method of claim 125 or 126, wherein the protein is an EGFR (ErbB-1), HER2 (ErbB-2), HER3 (ErbB-3), HER4 (Erb-4), BTK, Ras GTPase, KRas GTPase, or GPX4.
